(19) United States
(12) Patent Application Publication
(10) Pub. No.: US 2025/0304597 A1
(43) Pub. Date: Oct. 2, 2025

(54) COMPOSITIONS AND METHODS OF MODULATING SHORT-CHAIN DEHYDROGENASE ACTIVITY

(71) Applicants: CASE WESTERN RESERVE UNIVERSITY, Cleveland, OH (US); BOARD OF REGENTS OF THE UNIVERSITY OF TEXAS SYSTEM, Austin, TX (US)

(72) Inventors: Sanford Markowitz, Pepper Pike, OH (US); Monika Antczak, Fort Worth, TX (US); Joseph Ready, Carrollton, TX (US); Yongyou Zhang, Cleveland, OH (US)

(21) Appl. No.: 18/910,653

(22) Filed: Oct. 9, 2024

Related U.S. Application Data
(63) Continuation of application No. 18/057,589, filed on Nov. 21, 2022, now abandoned.
Application No. 18/057,589 is a continuation of application No. 16/617,137, filed on Nov. 26, 2019, now abandoned.
(60) Provisional application No. 62/511,674, filed on May 26, 2017.

(51) Int. Cl. C07D 495/04; C07D 495/22

(52) U.S. Cl. CPC C07D 495/04; C07D 495/22

(57) ABSTRACT

Compounds and methods of modulating 15-PGDH activity, modulating tissue prostaglandin levels, treating disease, diseases disorders, or conditions in which it is desired to modulate 15-PGDH activity and/or prostaglandin levels include 15-PGDH inhibitors described herein.

### RELATED APPLICATION

This application is a continuation of U.S. application Ser. No. 18/057,589, filed Nov. 21, 2022 (pending), which is a continuation of Ser. No. 16/617,137, filed Nov. 26, 2019 (now abandoned), which is a U.S. national stage application under 35 U.S.C. § 371 of International Application No. PCT/US2018/34944, filed May 29, 2018, which in turn claims priority from U.S. Provisional Application No. 62/511,674, filed May 26, 2017. The subject matter of each of the foregoing applications is incorporated herein by reference in their entireties.

### GOVERNMENT FUNDING

This invention was made with government support under Grant Nos. R01CA127306, R01CA127306-03S1, 1P01CA95471-10, AND 5P50CA150964, awarded by The National Institutes of Health. The United States government may have certain rights to the invention.

### BACKGROUND

Short-chain dehydrogenases (SCDs) are a family of dehydrogenases that share only 15% to 30% sequence identity, with similarity predominantly in the coenzyme binding domain and the substrate binding domain. In addition to their role in detoxification of ethanol, SCDs are involved in synthesis and degradation of fatty acids, steroids, and some prostaglandins, and are therefore implicated in a variety of disorders such as lipid storage disease, myopathy, SCD deficiency, and certain genetic disorders.

The SCD, 15-hydroxy-prostaglandin dehydrogenase (15-PGDH), (hydroxyprostaglandin dehydrogenase 15-(nicotinamide adeninedinucleotide); 15-PGDH; Enzyme Commission number 1.1.1.141; encoded by the HPGD gene), represents the key enzyme in the inactivation of a number of active prostaglandins, leukotrienes and hydroxyeicosatetraenoic acids (HETEs) (e.g., by catalyzing oxidation of PGE₂ to 15-keto-prostaglandin E2, 15k-PGE). The human enzyme is encoded by the HPGD gene and consists of a homodimer with subunits of a size of 29 kDa. The enzyme belongs to the evolutionarily conserved superfamily of short-chain dehydrogenase/reductase enzymes (SDRs), and according to the recently approved nomenclature for human enzymes, it is named SDR36CT. Thus far, two forms of 15-PGDH enzyme activity have been identified, NAD+-dependent type I 15-PGDH that is encoded by the HPGD gene, and the type II NADP-dependent 15-PGDH, also known as carbonyl reductase 1 (CBR1, SDR21C1). However, the preference of CBR1 for NADP and the high Km values of CBR1 for most prostaglandin suggest that the majority of the in vivo activity can be attributed to type I 15-PGDH encoded by the HPGD gene, that hereafter, and throughout all following text, simply denoted as 15-PGDH.

Recent studies suggest that inhibitors of 15-PGDH and activators of 15-PGDH could be therapeutically valuable. It has been shown that there is an increase in the incidence of colon tumors in 15-PGDH knockout mouse models. A more recent study implicates increased 15-PGDH expression in the protection of thrombin-mediated cell death. It is well known that 15-PGDH is responsible for the inactivation of prostaglandin E2 (PGE₂), which is a downstream product of COX-2 metabolism. PGE₂ has been shown to be beneficial in a variety of biological processes, such as hair density, dermal wound healing, and bone formation.

### SUMMARY

Embodiments described herein relate to compounds and methods of modulating short chain dehydrogenase (SCD) (e.g., 15-PGDH) activities, modulating tissue prostaglandin levels, and/or treating diseases, disorders, or conditions in which it is desired to modulate SCD (e.g., 15-PGDH) activity and/or prostaglandin levels.

In some embodiments, the modulator of SCD can be an SCD inhibitor that can be administered to tissue or blood of a subject at an amount effective to inhibit the activity of a short chain dehydrogenase enzyme. The SCD inhibitor can be a 15-PGDH inhibitor that can be administered to tissue or blood of a subject at an amount effective to increase prostaglandin levels in the tissue or blood. The 15-PGDH inhibitor can include a compound having the formula (I):

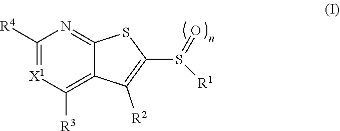

wherein n=0-2;
X¹ is N or CR^{a};
R¹ is selected from the group consisting of branched or linear alkyl including —(CH₂)n₁CH₃ (n₁=0-7),

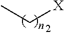

wherein n₂=0-6 and X is any of the following:
CF_{y}H_{z} (y+z=3), CCl_{y}H_{z} (y+z=3), OH, OAc, OMe, R⁷¹, OR⁷², CN, N(R⁷³)₂,

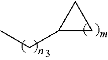

(n₃=0-5, m=1-5), and

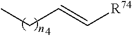

(n₄=0-5);
R² is selected from the group consisting of H, OH, linear or branched alkyl, Cl, F, NH₂, N(R⁷⁶)₂, and OR⁷⁷;
R³ and R⁴ are each independently selected from the group consisting of:

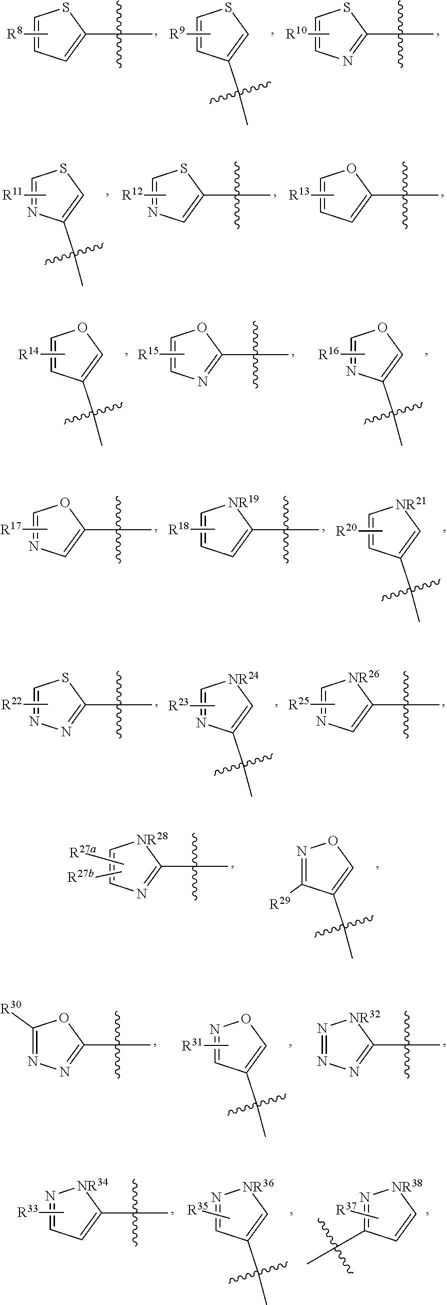

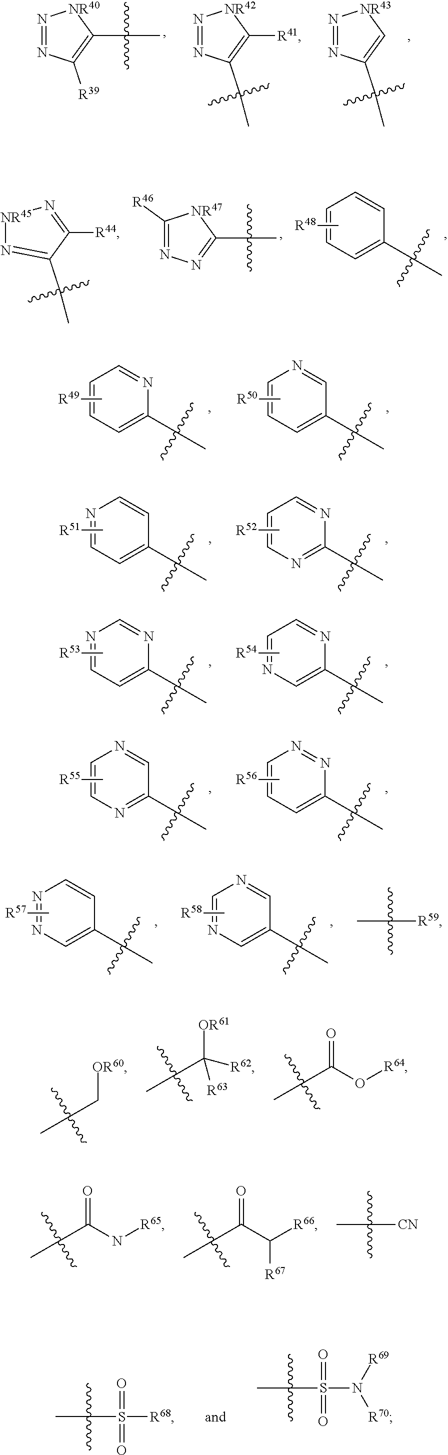

each R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁶, R⁷⁷, and R^{a} are the same or different and are independently selected from the group consisting of hydrogen, substituted or unsubstituted C₁-C₂₄ alkyl, C₂-C₂₄ alkenyl, C₂-C₂₄ alkynyl, C₆-C₂₀ aryl, heterocycloalkenyl containing from 4-6 ring atoms, (wherein from 1-3 of the ring atoms is independently selected from N, NH, N(C₁-C₆ alkyl), NC(O) (C₁-C₆ alkyl), O, and S), heteroaryl or heterocyclyl containing from 4-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano (—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, arlyl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R═H, methyl or other alkyl], groups incorporating amino acids or other moieties expected to bear positive or negative charge at physiological pH, and combinations thereof; wherein R² and R³ can be linked to form a cyclic or heterocyclic ring, and pharmaceutically acceptable salts thereof.

In some embodiments, X¹ can be N or CH. R⁴ can be a substituted or unsubstituted heterocyclyl containing 5-6 ring atoms. For example, R⁴ can be a substituted or unsubstituted thiophene, thiazole, oxazole, imidazole, pyridine, or phenyl.

In other embodiments, R⁴ can be selected from the group consisting of:

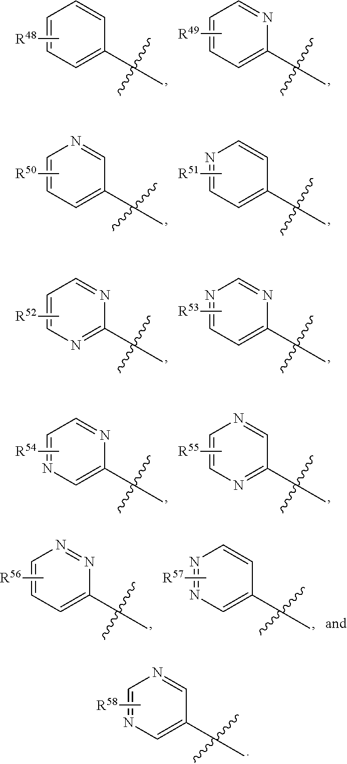

In still other embodiments, R³ is selected from the group consisting of H, substituted or unsubstituted aryl, a substituted or unsubstituted cycloalkyl, and a substituted or unsubstituted heterocyclyl, alkyl, or carboxy including carboxylic acid (—CO₂H), carboxy ester (—CO₂alkyl) and carboxamide [—CON(H)(alkyl) or —CO₂N(alkyl)₂].

In still other embodiments, the 15-PGDH inhibitor can include a compound having formula (II):

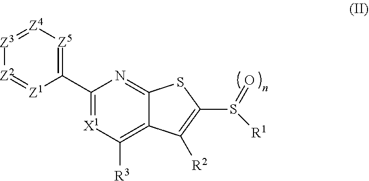

wherein n=0-2;
X¹ is N or CR^{a};
Z¹, Z², Z³, Z⁴, and Z⁵ are each independently N or CR^{b}, provided no more than 3 of Z¹, Z², Z³, Z⁴, and Z⁵ are N;
R¹ is selected from the group consisting of branched or linear alkyl including: —(CH₂)n₁CH₃ (n₁=0-7),

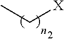

wherein n₂=0-6 and X is any of the following:
CF_{y}H_{z} (y+z=3), CCl_{y}H_{z} (y+z=3), OH, OAc, OMe, R⁷¹, OR⁷², CN, N(R⁷³)₂,

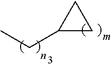

n₃=0-5, m=1-5), and

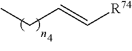

(n₄=0-5).
R² is selected from the group consisting of H, OH, Cl, F, NH₂, N(R⁷⁶)₂, and OR⁷⁷;
R³ is selected from the group consisting of:

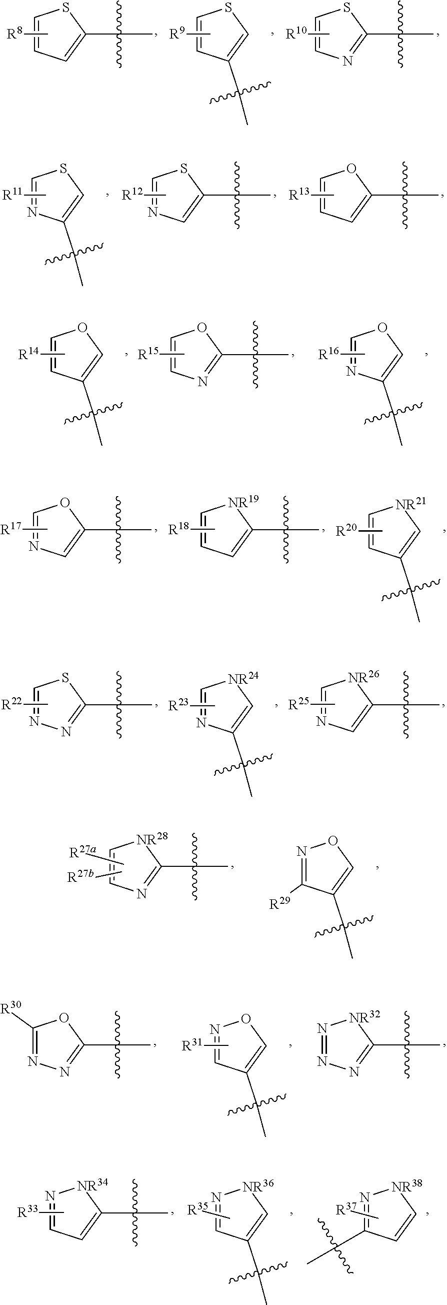

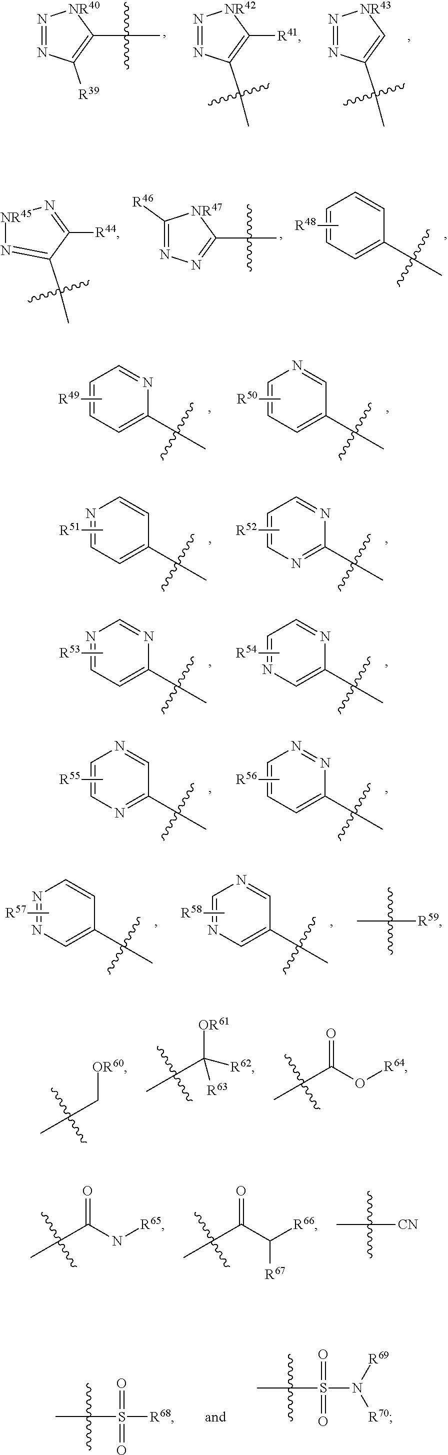

each R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁶, R⁷⁷, R^{a}, and R^{b} are the same or different and are independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 4-6 ring atoms, (wherein from 1-3 of the ring atoms is independently selected from N, NH, N(C₁-C₆ alkyl), NC(O)(C₁-C₆ alkyl), O, and S), heteroaryl or heterocyclyl containing from 4-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano (—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, arlyl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R═H, methyl or other alkyl], groups incorporating amino acids or other moieties expected to bear positive or negative charge at physiological pH, and combinations thereof; and pharmaceutically acceptable salts thereof; and wherein R³ is not

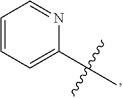 [SMILES: *c1ccccn1]

if one of Z¹ or Z⁵ is N and Z², Z³, and Z⁴ are CH.

In other embodiments, a compound having formula (II) does not have the following formula:

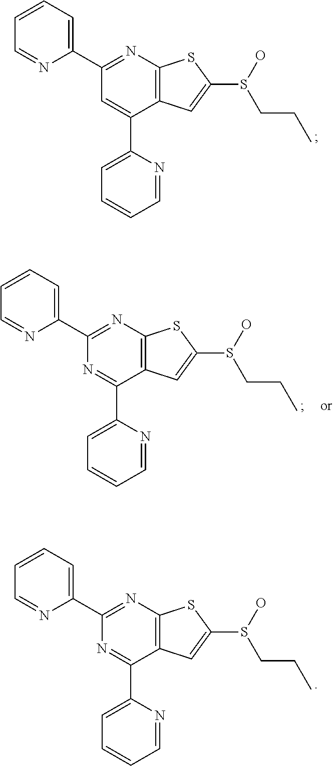

In some embodiments, R¹ is a —(CH₂)n₁CH₃ (n₁=1, 2, 3, 4, or 5) or

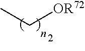

wherein n₂=1, 2, 3, 4, or 5.

In other embodiments, R² is —NH₂.

In other embodiments, R³ is:

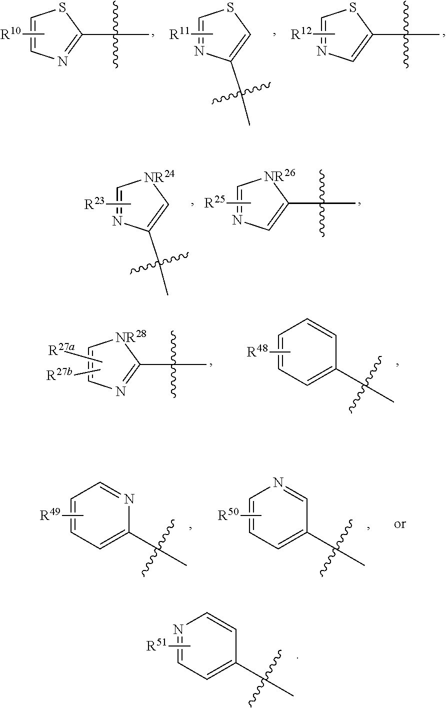

In some embodiments, each R¹⁰, R¹¹, R¹², R²³, R²⁴, R²⁵, R²⁶, R^{27a}, R^{27b}, R²⁸, R⁴⁸, R⁴⁹, R⁵⁰ and R⁵¹ are the same or different independently selected from hydrogen, halo, —C₁-C₆ alkyl, —C₃-C₆ cycloalkyl, and cyano.

In other embodiments, each R^{b} is the same or different and independently selected from hydrogen, halo, —NH₂, —NHC₁-C₃ alkyl, —N(C₁-C₃ alkyl)₂, —O—C₁-C₃ alkyl, and heterocyclyl containing from 4-6 ring atoms (wherein 1 atom of the ring atoms is independently selected from O), and pharmaceutically acceptable salts thereof.

In some embodiments, n=1.

In other embodiments, a compound having a structure according formula (II) can have a structure according to formula (IIA) to (IIH):

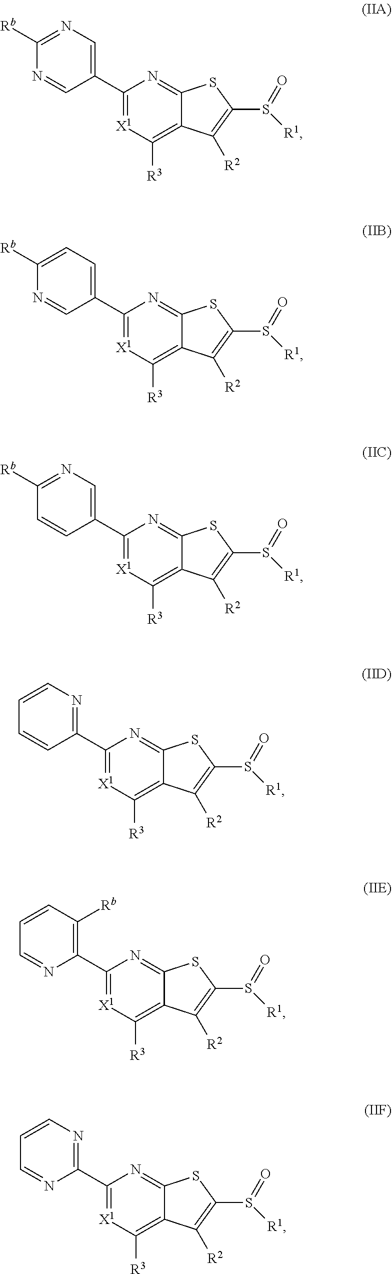

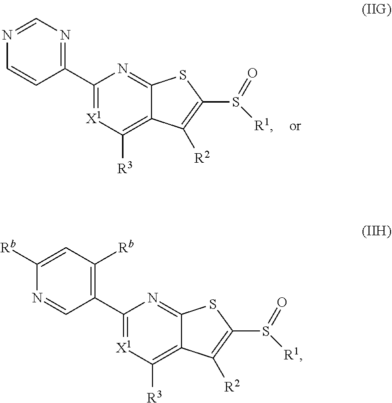

wherein:

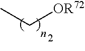

R¹ is a —(CH₂)n₁CH₃ (n₁=1, 2, 3, 4, or 5), or wherein n₂=1, 2, 3, 4, or 5;
R² is —NH₂
R³ is

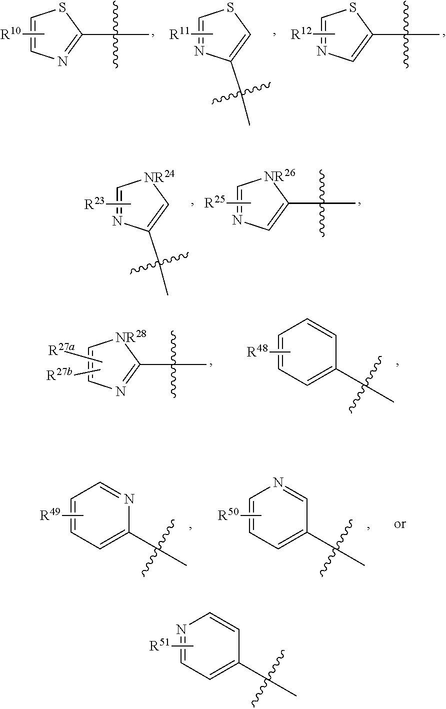

each of R¹⁰, R¹¹, R¹², R²³, R²⁴, R²⁵, R²⁶, R^{27a}, R^{27b}, R²⁸, R⁴⁸, R⁴⁹, and R⁵⁰ is the same or different and independently selected from hydrogen, halo, —C₁-C₃ alkyl, —C₃-C₆ cycloalkyl, or cyano;
each R^{b} is the same or different and independently selected from hydrogen, halo, —NH₂, —NHC₁-C₃ alkyl, —N(C₁-C₃ alkyl)₂, —O—C₁-C₃ alkyl, and heterocyclyl containing from 4-6 ring atoms (wherein 1 atom of the ring atoms is independently selected from O), and pharmaceutically acceptable salts thereof.

In other embodiments, a compound having a structure according formula (II) can have a structure according to one of formula (IIA) to (IIH):

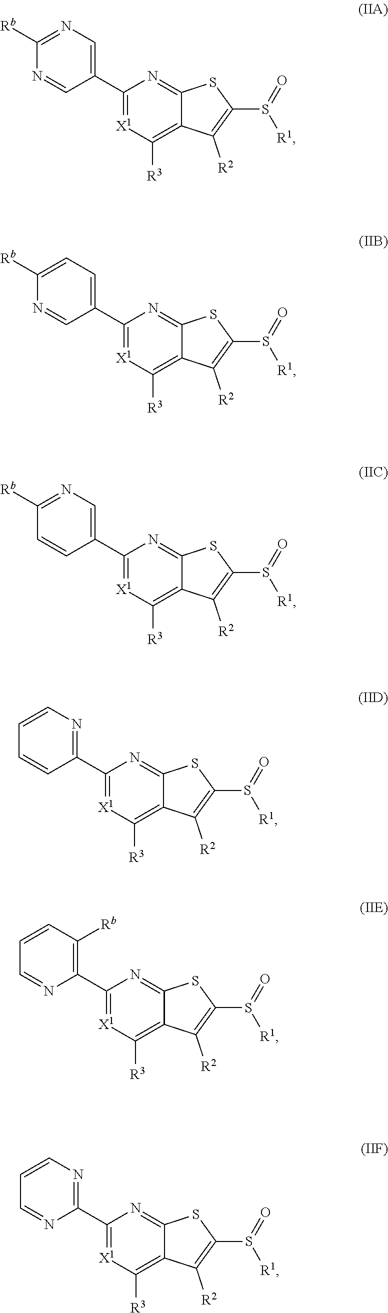

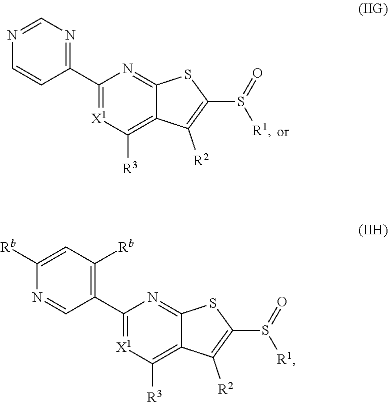

wherein:
R¹ is a —(CH₂)n₁CH₃ (n₁=1, 2, 3, 4, or 5), or

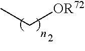

wherein n₂=1, 2, 3, 4, or 5;
R² is —NH₂′
R³ is

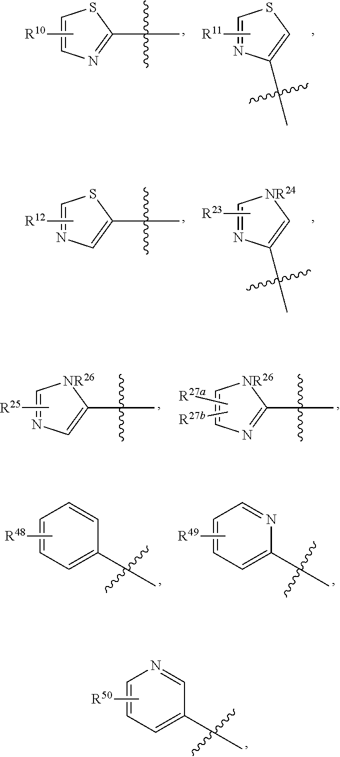

each R¹⁰, R¹¹, R¹², R²³, R²⁴, R²⁵, R²⁶, R^{27a}, R^{27b}, R²⁸, R⁴⁸, R⁴⁹, and R⁵⁰ is the same or different and independently selected from hydrogen, halo, —C₁-C₃ alkyl, —C₃-C₆ cycloalkyl, or cyano;
each R^{b} is the same or different and independently selected from hydrogen, halo, —NH₂, —NHC₁-C₆ alkyl, —N(C₁-C₆ alkyl)₂, —O—C₁-C₃ alkyl, heterocyclyl containing from 4-6 ring atoms, (wherein 1 atom of the ring atoms is O);
and pharmaceutically acceptable salts thereof.

In other embodiments, the 15-PGDH inhibitor can include a compound having a structure according formula (III):

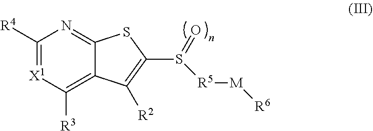

wherein n=0-2;
X¹ is N or CR^{a};
R² is selected from the group consisting of H, linear or branched alkyl, OH, Cl, F, NH₂, N(R⁷⁶)₂, and OR⁷⁷;
R³ and R⁴ are each independently selected from the group consisting of:

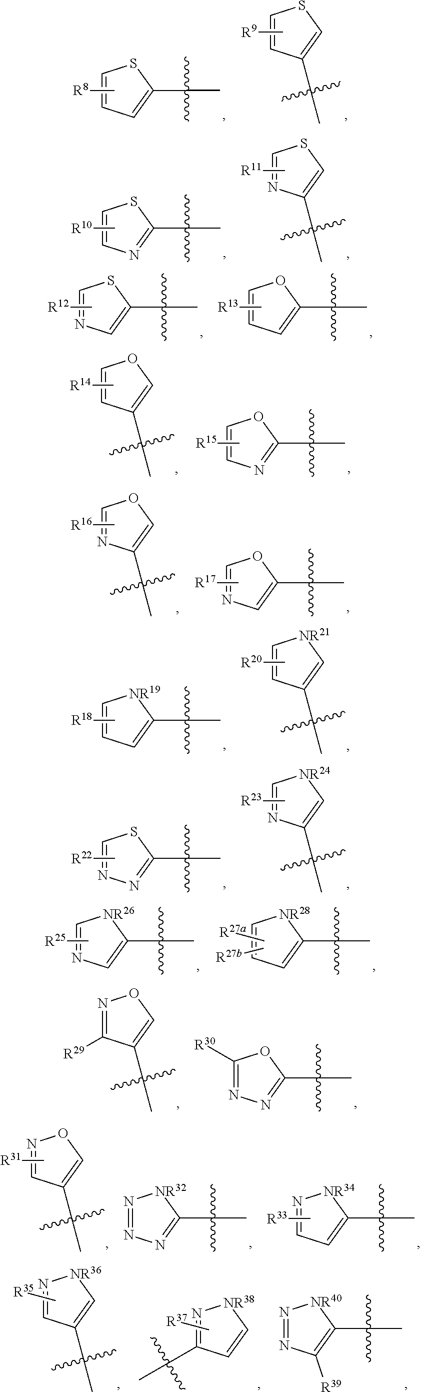

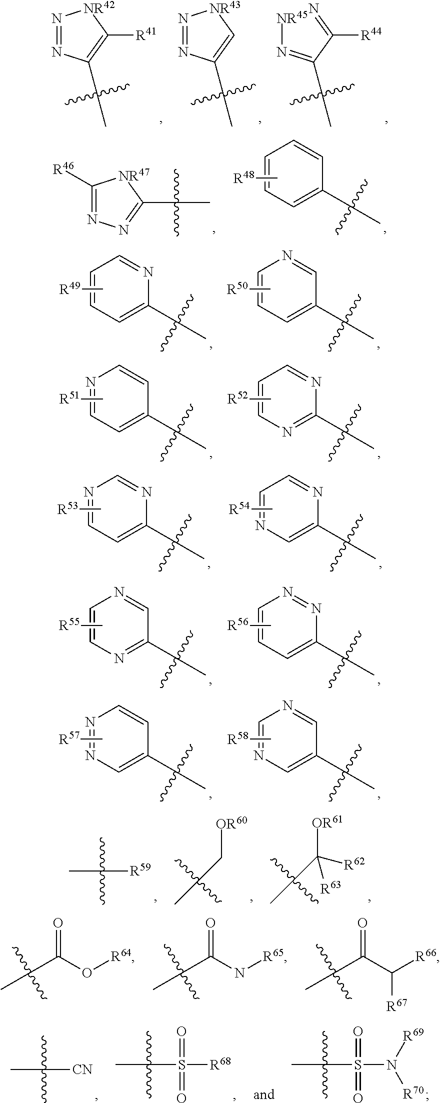

each R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷⁶, R⁷⁷, and R^{a} are the same or different and are independently selected from the group consisting of hydrogen, substituted or unsubstituted C₁-C₂₄ alkyl, C₂-C₂₄ alkenyl, C₂-C₂₄ alkynyl, C₆-C₂₀ aryl, heterocycloalkenyl containing from 4-6 ring atoms, (wherein from 1-3 of the ring atoms is independently selected from N, NH, N(C₁-C₆ alkyl), NC(O) (C₁-C₆ alkyl), O, and S), heteroaryl or heterocyclyl containing from 4-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano (—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R═hydrogen, alkyl, aryl, alkaryl, aralkyl, etc.), arylimino (—CR═N(aryl), where R═hydrogen, alkyl, aryl, alkaryl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, arlyl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R═H, methyl or other alkyl], groups incorporating amino acids or other moieties expected to bear positive or negative charge at physiological pH, and combinations thereof;
M is O or S;
R⁵ and R⁶ are each independently C₁-C₂₄ alkyl;
and pharmaceutically acceptable salts thereof.

In some embodiments, the compound of formula (III) is not a compound with a formula selected from the group consisting of:

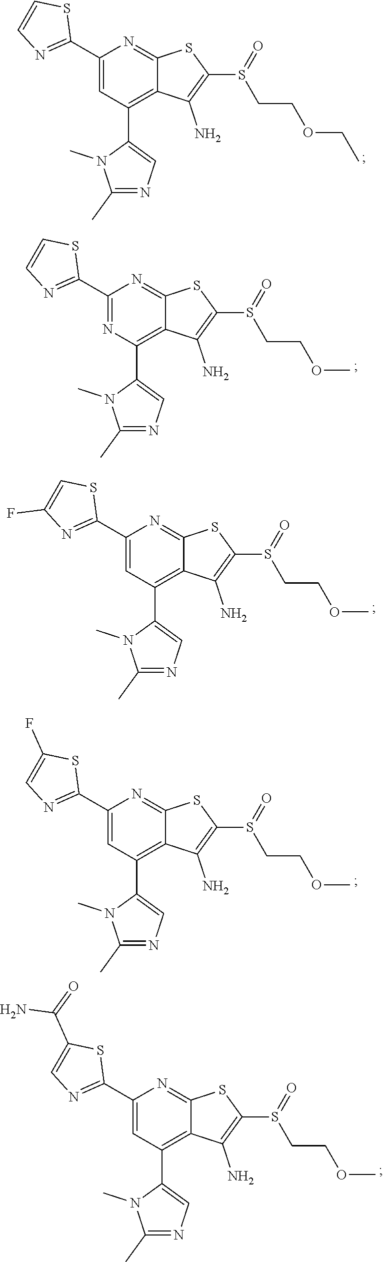

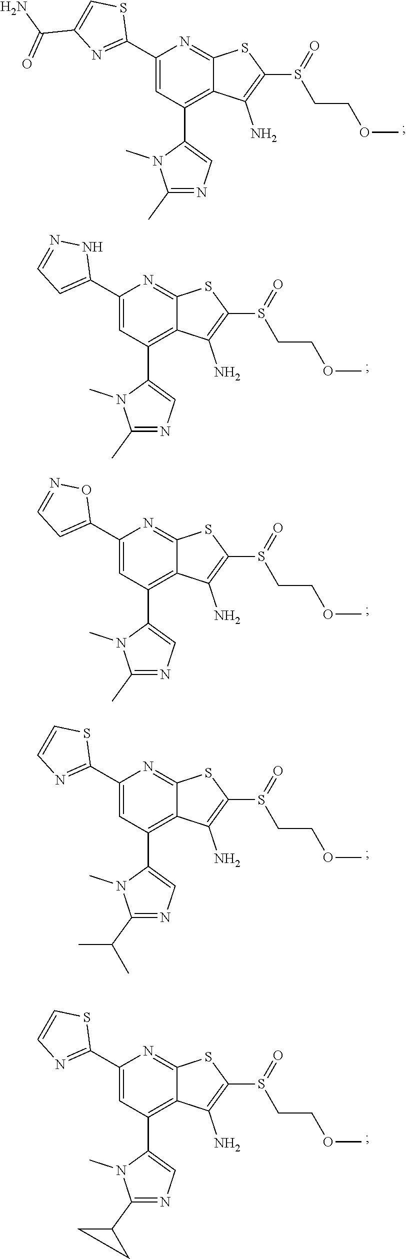

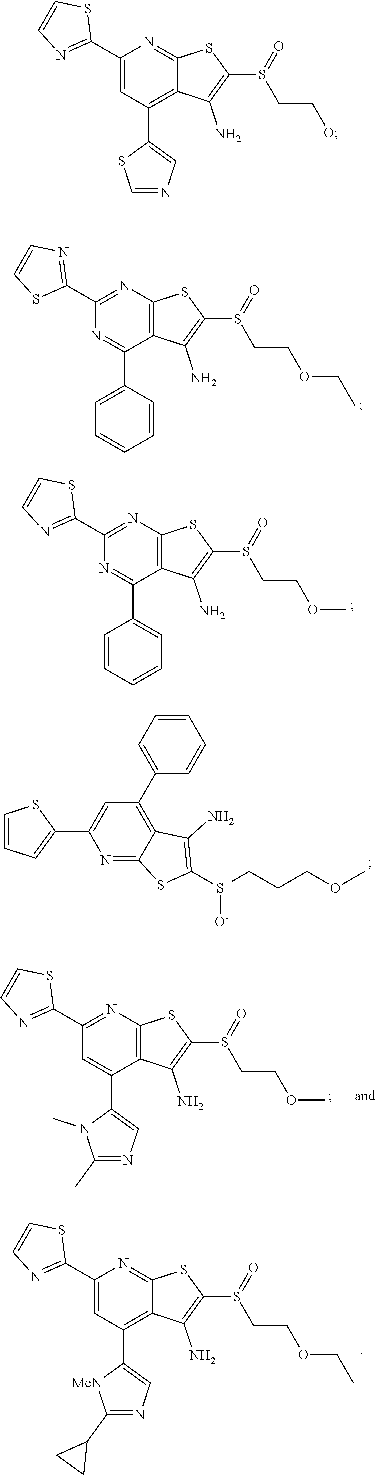

In some embodiments, R⁵ is a C₁-C₆ alkylene.

In other embodiments, R⁶ is a C₁-C₆ alkylene.

In still other embodiments, R⁵ is a C₂-C₅ alkylene and R⁶ is a C₁-C₃ alkylene.

In some embodiments, M is O.

In other embodiments, R² is NH₂.

In some embodiments, R³ and R⁴ are each independently

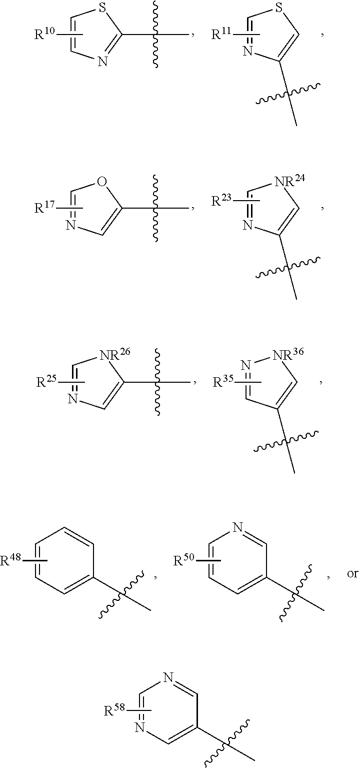

In some embodiments, each R¹⁰, R¹⁷, R²³, R²⁴, R²⁵, R²⁶, R³⁵, R³⁶, R⁴⁸, R⁵⁰, and R⁵⁸ is the same or different and independently hydrogen, halo, —C₁-C₃ alkyl, —C₃-C₆ cycloalkyl, heterocyclyl containing from 4-6 ring atoms, (wherein 1 atom of the ring atoms is O), or cyano.

In other embodiments, n=1.

In still other embodiments, R^{a} is H.

In other embodiments, the 15-PGDH inhibitor can include a compound having a structure according formula (IIIA):

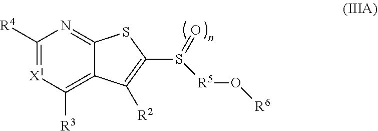

wherein n=1;
X¹ is N or CH;
R² is NH₂;
R³ and R⁴ are each independently

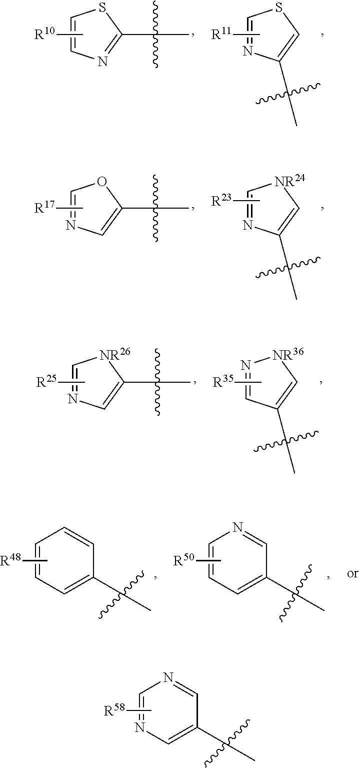

each R¹⁰, R¹⁷, R²³, R²⁴, R²⁵, R²⁶, R³⁵, R³⁶, R⁴⁸, R⁵⁰, and R⁵⁸ is the same or different and independently H, halo, —C₁-C₃ alkyl, —C₃-C₆ cycloalkyl, heterocyclyl containing from 4-6 ring atoms, (wherein 1 atom of the ring atoms is O), or cyano;
R⁵ and R⁶ are each independently C₁₋₆alkyl;
and pharmaceutically acceptable salts thereof.

In other embodiments, the 15-PGDH inhibitor can include a compound having a structure according to formula (IV):

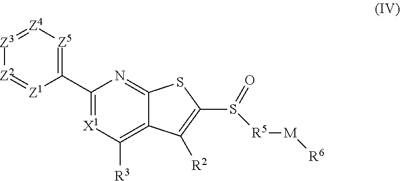

wherein n=0-2;
X¹ is N or CR^{a};
Z¹, Z², Z³, Z⁴, and Z⁵ are each independently N or CR^{b}, provided no more than 3 of Z¹, Z², Z³, Z⁴, and Z⁵ are N;
R² is selected from the group consisting of H, linear or branched alkyl, OH, Cl, F, NH₂, N(R⁷⁶)₂, and OR⁷⁷;
R³ is selected from the group consisting of:

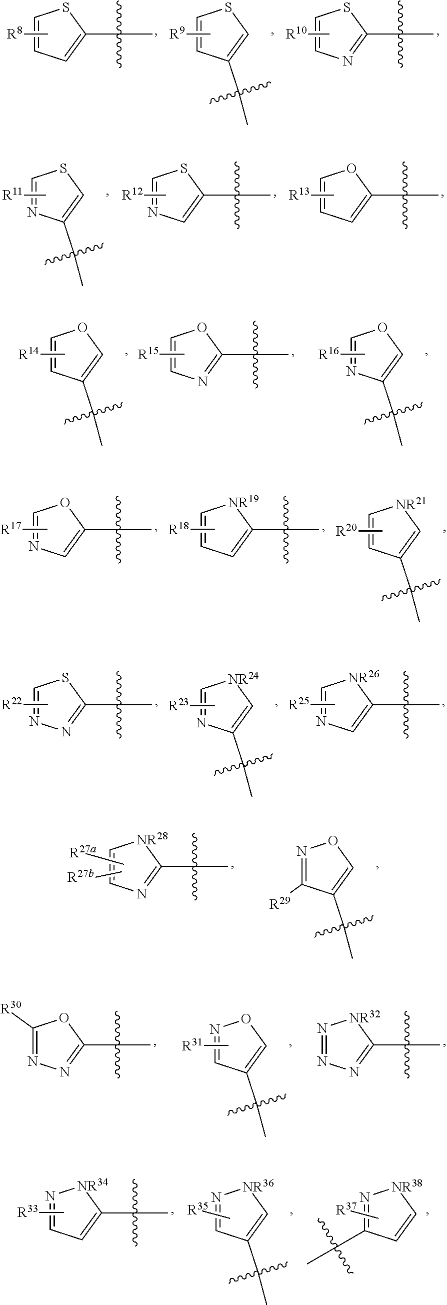

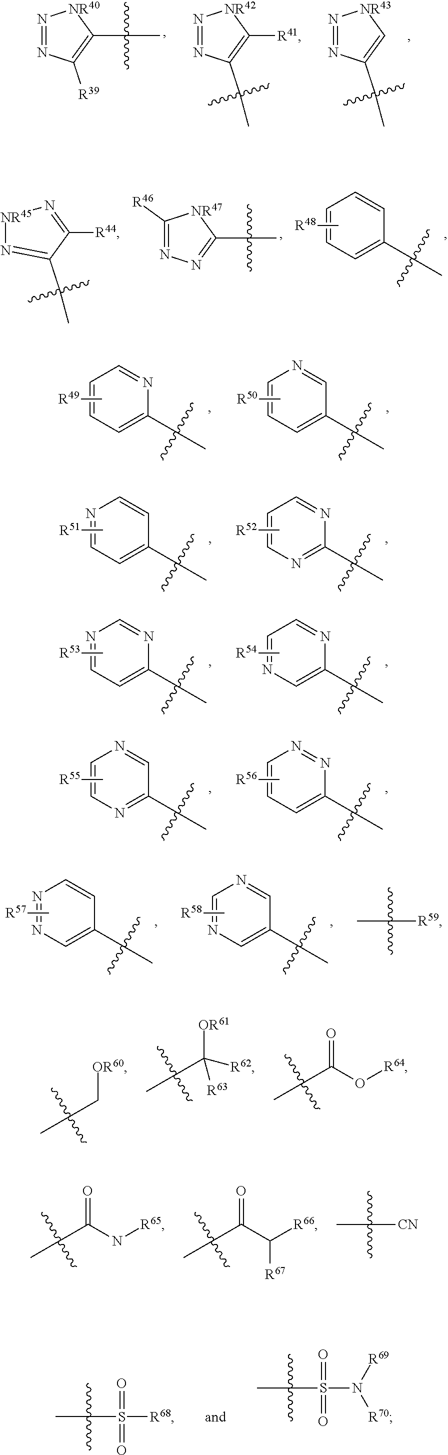

each R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷⁶, R⁷⁷, R^{a}, and R^{b} are the same or different and are independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 4-6 ring atoms, (wherein from 1-3 of the ring atoms is independently selected from N, NH, N(C₁-C₆ alkyl), NC(O)(C₁-C₆ alkyl), O, and S), heteroaryl or heterocyclyl containing from 4-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano(—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R═hydrogen, alkyl, aryl, alkaryl, aralkyl, etc.), arylimino (—CR═N(aryl), where R═hydrogen, alkyl, aryl, alkaryl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, arlyl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R═H, methyl or other alkyl], groups incorporating amino acids or other moieties expected to bear positive or negative charge at physiological pH, and combinations thereof;
M is O or S;
R⁵ and R⁶ are each independently C₁-C₂₄ alkylene;
and pharmaceutically acceptable salts thereof.

In some embodiments, R⁵ is a C₁-C₆ alkylene.

In other embodiments, R⁶ is a C₁-C₆ alkylene.

In some embodiments, R⁵ is a C₂-C₅ alkylene and R⁶ is a C₁-C₃ alkylene.

In other embodiments, M is O.

In still other embodiments, R² is H.

In other embodiments, R² is NH₂.

In some embodiments, R³ is

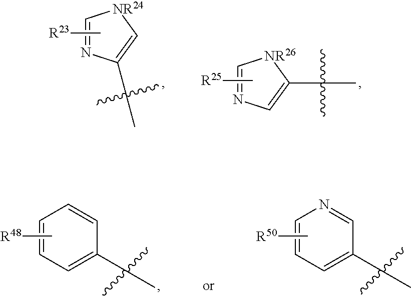

In other embodiments, each R²³, R²⁴, R²⁴, R²⁵, R⁴⁸, and R⁵⁰ are the same or different and independently selected hydrogen, halo, or C₁-C₃ alkyl.

In other embodiments, n=1.

In other embodiments, the 15-PGDH inhibitor can include a compound having the following structures:

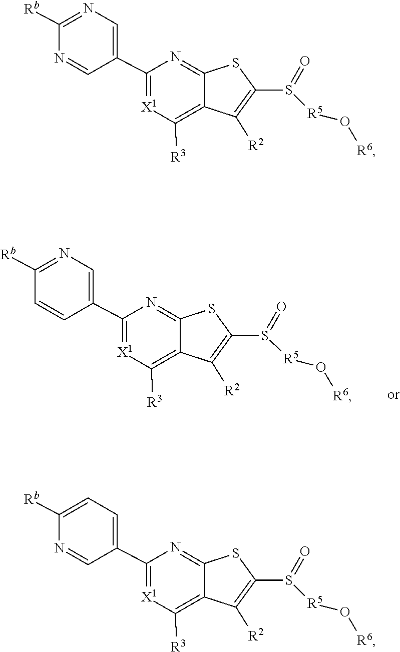

wherein:
R³ is

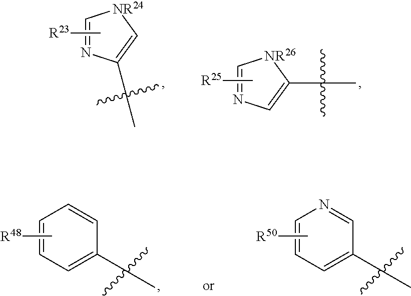

R⁵ is a C₂-C₅ alkyl and R⁶ is a C₁-C₃ alkylene;
each R²³, R²⁴, R²⁴, R²⁵, R⁴⁸, and R⁵⁰ are the same or different and independently selected hydrogen, halo, or C₁-C₃ alkyl;
each R^{b} is the same or different and independently selected from hydrogen, —NH₂, or heterocyclyl containing from 4-6 ring atoms (wherein 1 atom of the ring atoms is independently selected from O);
and pharmaceutically acceptable salts thereof.

In some embodiments, the 15-PGDH inhibitor can inhibit the enzymatic activity of recombinant 15-PGDH at an IC₅₀ of less than 1 μM, or preferably at an IC₅₀ of less than 250 nM, or more preferably at an IC₅₀ of less than 50 nM, or more preferably at an IC₅₀ of less than 10 nM, or more preferably at an IC₅₀ of less than 5 nM at a recombinant 15-PGDH concentration of about 5 nM to about 10 nM.

The 15-PGDH inhibitor can be provided in a topical composition that can be applied to skin of a subject to promote and/or stimulate pigmentation of the skin and/or hair growth and/or inhibiting hair loss, and/or treat skin damage or inflammation.

The 15-PGDH inhibitor can also be administered to a subject to promote wound healing, tissue repair, and/or tissue regeneration and/or engraftment or regeneration of a tissue graft.

In one embodiment, the 15-PGDH inhibitor can be administered to a subject to treat at least one of oral ulcers, gum disease, colitis, ulcerative colitis, gastrointestinal ulcers, inflammatory bowel disease, vascular insufficiency, Raynaud's disease, Buerger's disease, diabetic neuropathy, pulmonary artery hypertension, cardiovascular disease, and renal disease.

In another embodiment, the 15-PGDH inhibitor can be administered to a subject in combination with a prostanoid agonist for the purpose of enhancing the therapeutic effect of the agonist in prostaglandin responsive conditions.

In other embodiments, the 15-PGDH inhibitor can be administered to a subject and/or tissue of the subject to increase tissue stem cells. For example, the 15-PGDH inhibitor can be administered to bone marrow of a subject to increase stem cells in the subject.

In still other embodiments, the 15-PGDH inhibitor can be administered to a tissue graft donor, bone marrow graft donor, and/or a hematopoietic stem cell donor, and/or a tissue graft, and/or a bone marrow graft, and/or a hematopoietic stem cell graft, to increase the fitness of a donor tissue graft, a donor bone marrow graft, and/or a donor hematopoietic stem cell graft. For example, the 15-PGDH inhibitor can be administered to a subject, and/or bone marrow of a subject to increase the fitness of the marrow as a donor graft, and/or to a preparation of hematopoietic stem cells of a subject to increase the fitness of the stem cell preparation as a donor graft, and/or to a preparation of peripheral blood hematopoietic stem cells of a subject to increase the fitness of the stem cell preparation as a donor graft, and/or to a preparation of umbilical cord blood stem cells to increase the fitness of the stem cell preparation as a donor graft, and/or to a preparation of umbilical cord blood stem cells to decrease the number of units of umbilical cord blood required for transplantation.

In other embodiments, the 15-PGDH inhibitor can be administered to a subject to mitigate tissue graft rejection, to enhance tissue and/or bone marrow graft engraftment, to enhance bone marrow graft engraftment, following treatment of the subject or the marrow of the subject with radiation therapy, chemotherapy, or immunosuppressive therapy, to enhance engraftment of a progenitor stem cell graft, hematopoietic stem cell graft, or an umbilical cord blood stem cell graft, to enhance engraftment of a hematopoietic stem cell graft, or an umbilical cord stem cell graft, following treatment of the subject or the marrow of the subject with radiation therapy, chemotherapy, or immunosuppressive therapy, and/or in order to decrease the number of units of umbilical cord blood required for transplantation into the subject.

In other embodiments, the 15-PGDH inhibitor can be administered to a recipient of a tissue graft transplant, bone marrow transplant, and/or hematopoietic stem cell transplant, or of an umbilical cord stem cell transplant, in order to decrease the administration of other treatments or growth factors.

In some embodiments, the 15-PGDH inhibitor can be administered to a subject or to a tissue graft of a subject to mitigate graft rejection, to enhance graft engraftment, and/or to enhance graft engraftment following treatment of the subject or the marrow of the subject with radiation therapy, chemotherapy, or immunosuppressive therapy.

In other embodiments, the 15-PGDH inhibitor can be administered to a subject or to the bone marrow of a subject to confer resistance to toxic or lethal effects of exposure to radiation, to confer resistance to the toxic effect of Cytoxan, the toxic effect of fludarabine, the toxic effect of chemotherapy, or the toxic effect of immunosuppressive therapy, to decrease pulmonary toxicity from radiation, and/or to decrease infection.

In still other embodiments, the 15-PGDH inhibitor can be administered to a subject to increase neutrophil counts following a hematopoetic cell transplant with bone marrow, hematopoetic stem cells, or umbilical cord blood, to increase neutrophil counts in a subject with neutropia following chemotherapy administration or radiation therapy, to increase neutrophil counts in a subject with aplastic anemia, myelodysplasia, myelofibrosis, neutropenia due to other bone marrow diseases, drug induced neutropenia, autoimmune neutropenia, idiopathic neutropenia, or neutropenia following viral infections, to increase neutrophil counts in a subject with neutropia, to increase platelet counts following a hematopoetic cell transplant with bone marrow, hematopoetic stem cells, or umbilical cord blood, to increase platelet counts in a subject with thrombocytopenia following chemotherapy administration or radiation therapy, to increase platelet counts in a subject with aplastic anemia, myelodysplasia, myelofibrosis, thrombocytopenia due to other bone marrow diseases, drug induced thrombocytopenia, autoimmune thrombocytopenia, idiopathic thrombocytopenic purpura, idiopathic thrombocytopenia, or thrombocytopenia following viral infections, to increase platelet counts in a subject with thrombocytopenia, to increase red blood cell counts, or hematocrit, or hemoglobin level, following a hematopoetic cell transplant with bone marrow, hematopoetic stem cells, or umbilical cord blood, to increase red blood cell counts, or hematocrit, or hemoglobin level in a subject with anemia following chemotherapy administration or radiation therapy, to increase red blood cell counts, or hematocrit, or hemoglobin level counts in a subject with aplastic anemia, myelodysplasia, myelofibrosis, anemia due to other disorder of bone marrow, drug induced anemia, immune mediated anemias, anemia of chronic disease, anemia following viral infections, or anemia of unknown cause, to increase red blood cell counts, or hematocrit, or hemoglobin level in a subject with anemia, to increase bone marrow stem cells, following a hematopoetic cell transplant with bone marrow, hematopoetic stem cells, or umbilical cord blood, to increase bone marrow stem cells in a subject following chemotherapy administration or radiation therapy, and/or to increase bone marrow stem cells in a subject with aplastic anemia, myelodysplasia, myelofibrosis, other disorder of bone marrow, drug induced cytopenias, immune cytopenias, cytopenias following viral infections, or cytopenias.

In other embodiments, the administration of a 15-PGDH inhibitor can be used to modulate hematopoietic stem cells and hematopoiesis. For a 15-PGDH inhibitor can be administered alone or in combination with a cytokine to a subject in need thereof to increase and/or mobilize hematopoiectic stem cells and/or neutrophils in the blood, marrow, and/or tissue of the subject.

In some embodiments, the administration of a 15-PGDH inhibitor can be in combination with G-CSF for the purpose of increasing neutrophils.

In other embodiments, the administration of a 15-PGDH inhibitor can be in combination with a hematopoietic cytokine for the purpose of increasing neutrophils.

In still other embodiments, the administration of a 15-PGDH inhibitor can be in combination with G-CSF for the purpose of increasing numbers of and/or of mobilizing peripheral blood hematopoietic stem cells.

In other embodiments, the administration of a 15-PGDH inhibitor can be in combination with a hemopoietic cytokine for the purpose of increasing numbers of and/or of mobilizing peripheral blood hematopoietic stem cells.

In some embodiments, the administration of a 15-PGDH inhibitor can be in combination with a second agent, including Plerixafor, for the purpose of increasing numbers of and/or of mobilizing peripheral blood hematopoietic stem cells.

In other embodiments, the administration of a 15-PGDH inhibitor can be in combination with G-CSF for the purpose of increasing numbers of and/or of mobilizing peripheral blood hematopoietic stem cells for use in hematopoietic stem cell transplantation.

In still other embodiments, the administration of a 15-PGDH inhibitor can be in combination with a hemopoietic cytokine for the purpose of increasing numbers of and/or of mobilizing peripheral blood hematopoietic stem cells for use in hematopoietic stem cell transplantation.

In other embodiments, the administration of a 15-PGDH inhibitor can be in combination with a second agent, including Plerixafor, for the purpose of increasing numbers of and/or of mobilizing peripheral blood hematopoietic stem cells for use in hematopoietic stem cell transplantation.

In still other embodiments, the administration of a 15-PGDH inhibitor can be in combination with G-CSF for the purpose of increasing numbers of hematopoietic stem cells in blood or bone marrow.

In other embodiments, the administration of a 15-PGDH inhibitor can be in combination with a hemopoietic cytokine for the purpose of increasing numbers of hematopoietic stem cells in blood or bone marrow.

In other embodiments, the 15-PGDH inhibitor can be administered to a subject and/or tissue of the subject to increase tissue stem cells. For example, the 15-PGDH inhibitor can be administered to bone marrow of a subject to increase stem cells in the subject.

In still other embodiments, the 15-PGDH inhibitor can be administered to a tissue graft donor, bone marrow graft donor, and/or a hematopoietic stem cell donor, and/or a tissue graft, and/or a bone marrow graft, and/or a hematopoietic stem cell graft, to increase the fitness of a donor tissue graft, a donor bone marrow graft, and/or a donor hematopoietic stem cell graft. For example, the 15-PGDH inhibitor can be administered to a subject, and/or bone marrow of a subject to increase the fitness of the marrow as a donor graft, and/or to a preparation of hematopoietic stem cells of a subject to increase the fitness of the stem cell preparation as a donor graft, and/or to a preparation of peripheral blood hematopoietic stem cells of a subject to increase the fitness of the stem cell preparation as a donor graft, and/or to a preparation of umbilical cord blood stem cells to increase the fitness of the stem cell preparation as a donor graft, and/or to a preparation of umbilical cord blood stem cells to decrease the number of units of umbilical cord blood required for transplantation.

In other embodiments, the 15-PGDH inhibitor can be administered to a recipient of a tissue graft transplant, bone marrow transplant, and/or hematopoietic stem cell transplant, or of an umbilical cord stem cell transplant, in order to decrease the administration of other treatments or growth factors.

In still other embodiments, the 15-PGDH inhibitor can be administered to a subject to increase neutrophil counts following a hematopoetic cell transplant with bone marrow, hematopoetic stem cells, or umbilical cord blood, to increase neutrophil counts in a subject with neutropia following chemotherapy administration or radiation therapy, to increase neutrophil counts in a subject with aplastic anemia, myelodysplasia, myelofibrosis, neutropenia due to other bone marrow diseases, drug induced neutropenia, autoimmune neutropenia, idiopathic neutropenia, or neutropenia following viral infections, to increase neutrophil counts in a subject with neutropia, to increase platelet counts following a hematopoetic cell transplant with bone marrow, hematopoetic stem cells, or umbilical cord blood, to increase platelet counts in a subject with thrombocytopenia following chemotherapy administration or radiation therapy, to increase platelet counts in a subject with aplastic anemia, myelodysplasia, myelofibrosis, thrombocytopenia due to other bone marrow diseases, drug induced thrombocytopenia, autoimmune thrombocytopenia, idiopathic thrombocytopenic purpura, idiopathic thrombocytopenia, or thrombocytopenia following viral infections, to increase platelet counts in a subject with thrombocytopenia, to increase red blood cell counts, or hematocrit, or hemoglobin level, following a hematopoetic cell transplant with bone marrow, hematopoetic stem cells, or umbilical cord blood, to increase red blood cell counts, or hematocrit, or hemoglobin level in a subject with anemia following chemotherapy administration or radiation therapy, to increase red blood cell counts, or hematocrit, or hemoglobin level counts in a subject with aplastic anemia, myelodysplasia, myelofibrosis, anemia due to other disorder of bone marrow, drug induced anemia, immune mediated anemias, anemia of chronic disease, anemia following viral infections, or anemia of unknown cause, to increase red blood cell counts, or hematocrit, or hemoglobin level in a subject with anemia, to increase bone marrow stem cells, following a hematopoetic cell transplant with bone marrow, hematopoetic stem cells, or umbilical cord blood, to increase bone marrow stem cells in a subject following chemotherapy administration or radiation therapy, and/or to increase bone marrow stem cells in a subject with aplastic anemia, myelodysplasia, myelofibrosis, other disorder of bone marrow, drug induced cytopenias, immune cytopenias, cytopenias following viral infections, or cytopenias.

In other embodiments, the 15-PGDH inhibitor can be administered to a subject to increase responsiveness to cytokines in the presence of cytopenias, with cytopenias including any of: neutropenia, thrombocytopenia, lymphocytopenia and anemia; and with cytokines having increased responsiveness potentiated by the 15-PGDH inhibitor including any of: G-CSF, GM-CSF, EPO, IL-3, IL-6, TPO, TPO-RA (thrombopoietin receptor agonist), and SCF.

In some embodiments, the 15-PGDH inhibitor can be administered to a subject to increase bone density, treat osteoporosis, promote healing of fractures, or promote healing after bone surgery or joint replacement and/or to promote healing of bone to bone implants, bone to artificial implants, dental implants, and bone grafts.

In other embodiments, the 15-PGDH inhibitor can be administered to a subject or to the intestine of a subject to increase stem cells or cell proliferation in the intestine and/or and confer resistance to toxic or lethal effects of exposure to radiation or the toxic, lethal, or mucositis effects resultant from treatment with chemotherapy.

In some embodiments, the 15-PGDH inhibitor can be administered to a subject or to intestine of a subject as a treatment for colitis, ulcerative colitis, or inflammatory bowel disease.

In other embodiments, the 15-PGDH inhibitor can be administered to a subject to increase liver regeneration following liver surgery, following live liver donation, following liver transplantation, or following liver injury by toxins and/or to promote recovery from or resistance to liver toxins, including acetaminophen and related compounds.

In still other embodiments, the 15-PGDH inhibitor can be administered to a subject to treat erectile dysfunction.

In yet other embodiments, the 15-PGDH inhibitor can be administered to inhibit at least one of the growth, proliferation, or metastasis of 15-PGDH expressing cancers.

Still other embodiments described herein relate to a method of treating a subject in need of cell therapy. The method includes administering to the subject a therapeutically effective amount of a preparation comprising human hematopoietic stem cell administered a 15-PGDH inhibitor described herein and/or a therapeutic composition comprising human hematopoietic stem cells and a 15-PGDH inhibitor described herein.

In some embodiments, the subject has received human hematopoietic stem cells and/or has received the preparation and/or the therapeutic composition.

In other embodiments, the subject has acute myelogenous leukemia (AML), acute lymphoblastic leukemia (ALL), chronic myelogenous leukemia (CML), chronic lymphocytic leukemia (CLL), juvenile myelomonocytic leukemia, Hodgkin's lymphoma, non-Hodgkin's lymphoma, multiple myeloma, severe aplastic anemia, Fanconi's anemia, paroxysmal nocturnal hemoglobinuria (PNH), pure red cell aplasia, amegakaryocytosis/congenital thrombocytopenia, severe combined immunodeficiency syndrome (SCID), Wiskott-Aldrich syndrome, beta-thalassemia major, sickle cell disease, Hurler's syndrome, adrenoleukodystrophy, metachromatic leukodystrophy, myelodysplasia, refractory anemia, chronic myelomonocytic leukemia, agnogenic myeloid metaplasia, familial erythrophagocytic lymphohistiocytosis, solid tumors, chronic granulomatous disease, mucopolysaccharidoses, or Diamond Blackfan anemia.

Other embodiments relate to a method of treating a subject having at least one symptom associated with an ischemic tissue or a tissue damaged by ischemia. The method includes administering to the subject a therapeutically effective amount of a preparation comprising human hematopoietic stem cell administered a 15-PGDH inhibitor described herein and/or a therapeutic composition comprising human hematopoietic stem cells and a 15-PGDH inhibitor described herein.

In some embodiments, the ischemia can be associated with at least one of acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetic neuropathy, diabetes induced ischemia, disseminated intravascular coagulation (DIC), embolic brain ischemia, graft-versus-host disease, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient isc hemic attack (TIA), traumatic brain injury, ulcerative colitis, vascular disease of the kidney, vascular inflammatory conditions, von Hippel-Lindau syndrome, and wounds to tissues or organs.

Other embodiments relate to methods for treating and/or preventing fibrosis and various fibrotic diseases, disorders or conditions by administration of 15-PGDH inhibitors. In some embodiments, a 15-PGDH inhibitor described herein can be administered to a subject in need thereof to decrease fibrotic symptoms, such as collagen deposition, inflammatory cytokine expression, and inflammatory cell infiltration, and treat and/or prevent various fibrotic diseases, disorders, and conditions characterized, in whole or in part, by the excess production of fibrous material, including excess production of fibrotic material within the extracellular matrix, or the replacement of normal tissue elements by abnormal, non-functional, and/or excessive accumulation of matrix-associated components.

Fibrotic diseases, disorders and conditions characterized, in whole or in part, by excess production of fibrotic material can include systemic sclerosis, multifocal fibrosclerosis, nephrogenic systemic fibrosis, scleroderma (including morphea, generalized morphea, or linear scleroderma), sclerodermatous graft-vs-host-disease, kidney fibrosis (including glomerular sclerosis, renal tubulointerstitial fibrosis, progressive renal disease or diabetic nephropathy), cardiac fibrosis (e.g., myocardial fibrosis), pulomanry fibrosis (e.g., glomerulosclerosis pulmonary fibrosis, idiopathic pulmonary fibrosis, silicosis, asbestosis, interstitial lung disease, interstitial fibrotic lung disease, and chemotherapy/radiation induced pulmonary fibrosis), oral fibrosis, endomyocardial fibrosis, deltoid fibrosis, pancreatitis, inflammatory bowel disease, Crohn's disease, nodular fascilitis, eosinophilic fasciitis, general fibrosis syndrome characterized by replacement of normal muscle tissue by fibrous tissue in varying degrees, retroperitoneal fibrosis, liver fibrosis, liver cirrhosis, chronic renal failure; myelofibrosis (bone marrow fibrosis), drug induced ergotism, glioblastoma in Li-Fraumeni syndrome, sporadic glioblastoma, myleoid leukemia, acute myelogenous leukemia, myelodysplastic syndrome, myeloproferative syndrome, gynecological cancer, Kaposi's sarcoma, Hansen's disease, collagenous colitis, acute fibrosis, organ specific fibrosis, and the like.

In some embodiments, a method of treating or preventing a fibrotic disease, disorder or condition includes administering to a subject in need thereof a therapeutically effect amount of a 15-PGDH inhibitor.

In some embodiments, the 15-PGDH inhibitors can be used to treat or prevent lung fibrosis. Lung fibrosis, which can be treated, can be selected from the group consisting of pulmonary fibrosis, pulmonary hypertension, chronic obstructive pulmonary disease (COPD), asthma, idiopathic pulmonary fibrosis, sarcoidosis, cystic fibrosis, familial pulmonary fibrosis, silicosis, asbestosis, coal worker's pneumoconiosis, carbon pneumoconiosis, hypersensitivity pneumonitides, pulmonary fibrosis caused by inhalation of inorganic dust, pulmonary fibrosis caused by an infectious agent, pulmonary fibrosis caused by inhalation of noxious gases, aerosols, chemical dusts, fumes or vapors, drug-induced interstitial lung disease, or pulmonary hypertension, and combinations thereof.

In other embodiments, the 15-PGDH inhibitors can be used to treat or prevent kidney fibrosis. The kidney fibrosis can result from dialysis following kidney failure, catheter placement, a nephropathy, glomerulosclerosis, glomerulonephritis, chronic renal insufficiency, acute kidney injury, end stage renal disease or renal failure, or combinations thereof.

In other embodiments, the 15-PGDH inhibitors can be used to treat or prevent liver fibrosis. The liver fibrosis can result from a chronic liver disease, viral induced hepatic cirrhosis, hepatitis B virus infection, hepatitis C virus infection, hepatitis D virus infection, schistosomiasis, primary biliary cirrhosis, alcoholic liver disease or non-alcoholic steatohepatitis (NASH), NASH associated cirrhosis obesity, diabetes, protein malnutrition, coronary artery disease, auto-immune hepatitis, cystic fibrosis, alpha-1-antitrypsin deficiency, primary biliary cirrhosis, drug reaction and exposure to toxins, or combinations thereof.

In some embodiments, the 15-PGDH inhibitors can be used to treat or prevent heart fibrosis, for example, cardiac fibrosis and endomyocardial fibrosis.

In some embodiments, the 15-PGDH inhibitors can be used to treat or prevent systemic sclerosis.

In some embodiments, the 15-PGDH inhibitors can be used to treat or prevent fibrotic diseases, disorders or conditions caused by post-surgical adhesion formation.

In some embodiments, the 15-PGDH inhibitors can be used for reducing or preventing scar formation in a subject.

In other embodiments, the 15-PGDH inhibitors can be used to reduce or prevent scar formation on skin or scleroderma.

In various embodiments, the 15-PGDH inhibitors can be administered at a therapeutically effective amount such that at least one symptom or feature of a fibrotic disease, disorder or condition, or other related diseases, disorders or conditions, is reduced in intensity, severity, or frequency, or has delayed onset.

In other embodiments, the 15-PGDH inhibitors can be used in a method for decreasing or reducing collagen secretion or collagen deposition in a tissue or organ, such as the lung, the liver, the intestines, the colon, the skin or the heart, of a subject. The method can include administering a therapeutically effective amount of the 15-PGDH inhibitors to the subject in need thereof. The subject can have or be at risk of an excessive collagen secretion or collagen deposition in the tissue or organ, such as the kidney, the lung, the liver, the intestines, the colon, the skin or the heart. Usually, the excessive collagen secretion or collagen deposition in an organ results from an injury or an insult. Such injury and insult can be organ-specific. The 15-PGDH inhibitors can be administered over a sufficient period of time to decrease or reduce the level of collagen deposition in the tissue or organ, completely or partially. A sufficient period of time can be during one week, or between 1 week to 1 month, or between 1 to 2 months, or 2 months or more. For chronic condition, the 15-PGDH inhibitors can be advantageously administered for life time period.

Other embodiments described herein relate to the use of 15-PGDH inhibitors in combination with corticosteroids or TNF inhibitors to treat inflammation, reduce aberrant activity of the immune system, and/or promote wound healing in a subject in need thereof. It was found that corticosteroids administered to a subject can induce 15-PGDH expression in tissue of the subject. Administration of a 15-PGDH inhibitor in combination with a corticosteroid was found to enhance anti-inflammatory and/or immunosuppressive effects of the corticosteroid while attenuating corticosteroid induced adverse and/or cytotoxic effects. Treatment of inflammatory, disorders, immune disorders, and/or wounds by administration of 15-PGDH inhibitors in combination with corticosteroids can increase therapeutic efficacy and can allow the corticosteroids to be administered, in some instances, at lower dosages to achieve similar effects, and, in other instances, at higher dosages and for prolonged periods of times with attenuated and/or reduced adverse or cytotoxic effects.

In some embodiments, the inflammatory and/or immune disease or disorder treated with the combination of 15-PGDH inhibitor and a corticosteroid or TNF inhibitor can include intestinal, gastrointestinal, or bowel disorders. As described below, it was found that inhibitors of short-chain dehydrogenase activity, such as 15-PGDH inhibitors, can be administered to a subject in need thereof alone or in combination with corticosteroids and tumor necrosis factor (TNF)-alpha antagonists to treat intestinal, gastrointestinal, or bowel disorders, such as oral ulcers, gum disease, gastritis, colitis, ulcerative colitis, gastric ulcers, inflammatory bowel disease, and Crohn's disease.

In other embodiments, the 15-PGDH inhibitor can be used as a glucocorticoid sensitizer to treat glucocorticoid insensitivity, restore corticosteroid sensitivity, enhance glucocorticoid sensitivity, and/or reverse the glucocorticoid insensitivity in a subject experiencing corticosteroid dependence or corticoid resistance or unresponsiveness or intolerance to corticosteroids. For example, the 15-PGDH inhibitor can be administered to a subject in combination with the corticosteroid to treat glucocorticoid insensitivity, restore corticosteroid sensitivity, enhance glucocorticoid sensitivity, and/or reverse the glucocorticoid insensitivity in a subject experiencing corticosteroid dependence or corticoid resistance or unresponsiveness or intolerance to corticosteroids.

The 15-PGDH inhibitor can also be administered in combination with a corticosteroid or TNF inhibitor to a subject to promote wound healing, tissue repair, and/or tissue regeneration and/or engraftment or regeneration of a tissue graft.

In some embodiments, the 15-PGDH inhibitor can be administered to a subject at an amount effective to increase prostaglandin levels in the subject and attenuate corticosteroid induced adverse and/or cytotoxic effects.

### BRIEF DESCRIPTION OF THE DRAWINGS

FIG. 1 illustrates a graph showing the dose response of compound 1 (2-(butylsulfinyl)-4-(2-cyclopropyl-1-methyl-1H-imidazol-5-yl)-6-(thiazol-2-yl)thieno[2,3-b]pyridin-3-amine) and compound 2 (compound 52A in Table 1; (R)-2-(butylsulfinyl)-4-(2-cylcopropyl-1-methyl-1H-imidazol-5-yl)-6-(pyridine-3-yl)thieno[2,3-b]pyridine-3-amine), on PGE2 production of A549 cells stimulated with IL-1β.

FIG. 2 illustrates a liquid chromatograpy-mass spectrometry (LC-MS) chromatogram of 2-(butylsulfinyl)-4-(1,2-dimethyl-1H-imidazol-5-yl)-6-(thiazol-2-yl)thieno[2,3-b]pyridin-3-amine and its metabolites following incubation in the presence of mouse liver microsomes.

FIG. 3 illustrates a liquid chromatograpy-mass spectrometry (LC-MS) chromatogram of 2-(butylsulfinyl)-4-(1,2-dimethyl-1H-imidazol-5-yl)-6-(thiazol-2-yl)thieno[2,3-b]pyridin-3-amine and its metabolites following incubation in the presence of mouse liver microsomes.

### DETAILED DESCRIPTION

For convenience, certain terms employed in the specification, examples, and appended claims are collected here. Unless defined otherwise, all technical and scientific terms used herein have the same meaning as commonly understood by one of ordinary skill in the art to which this application belongs.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably.

The term “or” as used herein should be understood to mean “and/or”, unless the context clearly indicates otherwise.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

It will be noted that the structure of some of the compounds of the application include asymmetric (chiral) carbon or sulfur atoms. It is to be understood accordingly that the isomers arising from such asymmetry are included herein, unless indicated otherwise. Such isomers can be obtained in substantially pure form by classical separation techniques and by stereochemically controlled synthesis. The compounds of this application may exist in stereoisomeric form, therefore can be produced as individual stereoisomers or as mixtures.

The term “isomerism” means compounds that have identical molecular formulae but that differ in the nature or the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images are termed “enantiomers”, or sometimes optical isomers. A carbon atom bonded to four nonidentical substituents is termed a “chiral center” whereas a sulfur bound to three or four different substitutents, e.g. sulfoxides or sulfinimides, is likewise termed a “chiral center”.

The term “chiral isomer” means a compound with at least one chiral center. It has two enantiomeric forms of opposite chirality and may exist either as an individual enantiomer or as a mixture of enantiomers. A mixture containing equal amounts of individual enantiomeric forms of opposite chirality is termed a “racemic mixture”. A compound that has more than one chiral center has 2n−1 enantiomeric pairs, where n is the number of chiral centers. Compounds with more than one chiral center may exist as either an individual diastereomer or as a mixture of diastereomers, termed a “diastereomeric mixture”. When one chiral center is present, a stereoisomer may be characterized by the absolute configuration (R or S) of that chiral center. Alternatively, when one or more chiral centers are present, a stereoisomer may be characterized as (+) or (−). Absolute configuration refers to the arrangement in space of the substituents attached to the chiral center. The substituents attached to the chiral center under consideration are ranked in accordance with the Sequence Rule of Cahn, Ingold and Prelog. (Cahn et al, Angew. Chem. Inter. Edit. 1966, 5, 385; errata 511; Cahn et al., Angew. Chem. 1966, 78, 413; Cahn and Ingold, J Chem. Soc. 1951 (London), 612; Cahn et al., Experientia 1956, 12, 81; Cahn, J., Chem. Educ. 1964, 41, 116).

The term “geometric Isomers” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules. Further, the structures and other compounds discussed in this application include all atropic isomers thereof.

The term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

The terms “crystal polymorphs” or “polymorphs” or “crystal forms” means crystal structures in which a compound (or salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

The term “derivative” refers to compounds that have a common core structure, and are substituted with various groups as described herein.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include acyl sulfonimides, tetrazoles, sulfonates, and phosphonates. See, e.g., Patani and LaVoie, Chem. Rev. 96, 3147-3176 (1996).

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The term “treating” is art-recognized and includes inhibiting a disease, disorder or condition in a subject, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The term “preventing” is art-recognized and includes stopping a disease, disorder or condition from occurring in a subject, which may be predisposed to the disease, disorder and/or condition but has not yet been diagnosed as having it. Preventing a condition related to a disease includes stopping the condition from occurring after the disease has been diagnosed but before the condition has been diagnosed.

The term “pharmaceutical composition” refers to a formulation containing the disclosed compounds in a form suitable for administration to a subject. In a preferred embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient (e.g., a formulation of the disclosed compound or salts thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, intranasal, inhalational, and the like. Dosage forms for the topical or transdermal administration of a compound described herein includes powders, sprays, ointments, pastes, creams, lotions, gels, solutions, patches, nebulized compounds, and inhalants. In a preferred embodiment, the active compound is mixed under sterile conditions with a pharmaceutically acceptable carrier, and with any preservatives, buffers, or propellants that are required.

The term “flash dose” refers to compound formulations that are rapidly dispersing dosage forms.

The term “immediate release” is defined as a release of compound from a dosage form in a relatively brief period of time, generally up to about 60 minutes. The term “modified release” is defined to include delayed release, extended release, and pulsed release. The term “pulsed release” is defined as a series of releases of drug from a dosage form. The term “sustained release” or “extended release” is defined as continuous release of a compound from a dosage form over a prolonged period.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, excipient, solvent or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The compounds of the application are capable of further forming salts. All of these forms are also contemplated herein.

“Pharmaceutically acceptable salt” of a compound means a salt that is pharmaceutically acceptable and that possesses the desired pharmacological activity of the parent compound. For example, the salt can be an acid addition salt. One embodiment of an acid addition salt is a hydrochloride salt. The pharmaceutically acceptable salts can be synthesized from a parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile being preferred. Lists of salts are found in Remington's Pharmaceutical Sciences, 18th ed. (Mack Publishing Company, 1990).

The compounds described herein can also be prepared as esters, for example pharmaceutically acceptable esters. For example, a carboxylic acid function group in a compound can be converted to its corresponding ester, e.g., a methyl, ethyl, or other ester. Also, an alcohol group in a compound can be converted to its corresponding ester, e.g., an acetate, propionate, or other ester.

The compounds described herein can also be prepared as prodrugs, for example pharmaceutically acceptable prodrugs. The terms “pro-drug” and “prodrug” are used interchangeably herein and refer to any compound, which releases an active parent drug in vivo. Since prodrugs are known to enhance numerous desirable qualities of pharmaceuticals (e.g., solubility, bioavailability, manufacturing, etc.) the compounds can be delivered in prodrug form. Thus, the compounds described herein are intended to cover prodrugs of the presently claimed compounds, methods of delivering the same and compositions containing the same. “Prodrugs” are intended to include any covalently bonded carriers that release an active parent drug in vivo when such prodrug is administered to a subject. Prodrugs are prepared by modifying functional groups present in the compound in such a way that the modifications are cleaved, either in routine manipulation or in vivo, to the parent compound. Prodrugs include compounds wherein a hydroxy, amino, sulfhydryl, carboxy, or carbonyl group is bonded to any group that may be cleaved in vivo to form a free hydroxyl, free amino, free sulfhydryl, free carboxy or free carbonyl group, respectively. Prodrugs can also include a precursor (forerunner) of a compound described herein that undergoes chemical conversion by metabolic processes before becoming an active or more active pharmacological agent or active compound described herein.

Examples of prodrugs include, but are not limited to, esters (e.g., acetate, dialkylaminoacetates, formates, phosphates, sulfates, and benzoate derivatives) and carbamates (e.g., N,N-dimethylaminocarbonyl) of hydroxy functional groups, ester groups (e.g., ethyl esters, morpholinoethanol esters) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in compounds, and the like, as well as sulfides that are oxidized to form sulfoxides or sulfones.

The term “protecting group” refers to a grouping of atoms that when attached to a reactive group in a molecule masks, reduces or prevents that reactivity. Examples of protecting groups can be found in Green and Wuts, Protective Groups in Organic Chemistry, (Wiley, 2.sup.nd ed. 1991); Harrison and Harrison et al., Compendium of Synthetic Organic Methods, Vols. 1-8 (John Wiley and Sons, 1971-1996); and Kocienski, Protecting Groups, (Verlag, 3^{rd} ed. 2003).

The term “amine protecting group” is intended to mean a functional group that converts an amine, amide, or other nitrogen-containing moiety into a different chemical group that is substantially inert to the conditions of a particular chemical reaction. Amine protecting groups are preferably removed easily and selectively in good yield under conditions that do not affect other functional groups of the molecule. Examples of amine protecting groups include, but are not limited to, formyl, acetyl, benzyl, t-butyldimethylsilyl, t-butyldiphenylsilyl, t-butyloxycarbonyl (Boc), p-methoxybenzyl, methoxymethyl, tosyl, trifluoroacetyl, trimethylsilyl (TMS), fluorenyl-methyloxycarbonyl, 2-trimethylsilyl-ethyoxycarbonyl, 1-methyl-1-(4-biphenylyl) ethoxycarbonyl, allyloxycarbonyl, benzyloxycarbonyl (CBZ), 2-trimethylsilyl-ethanesulfonyl (SES), trityl and substituted trityl groups, 9-fluorenylmethyloxycarbonyl (FMOC), nitro-veratryloxycarbonyl (NVOC), and the like. Those of skill in the art can identify other suitable amine protecting groups.

Representative hydroxy protecting groups include those where the hydroxy group is either acylated or alkylated such as benzyl, and trityl ethers as well as alkyl ethers, tetrahydropyranyl ethers, trialkylsilyl ethers and allyl ethers.

Additionally, the salts of the compounds described herein, can exist in either hydrated or unhydrated (the anhydrous) form or as solvates with other solvent molecules. Non-limiting examples of hydrates include monohydrates, dihydrates, etc. Nonlimiting examples of solvates include ethanol solvates, acetone solvates, etc.

The term “solvates” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

The compounds, salts and prodrugs described herein can exist in several tautomeric forms, including the enol and imine form, and the keto and enamine form and geometric isomers and mixtures thereof. Tautomers exist as mixtures of a tautomeric set in solution. In solid form, usually one tautomer predominates. Even though one tautomer may be described, the present application includes all tautomers of the present compounds. A tautomer is one of two or more structural isomers that exist in equilibrium and are readily converted from one isomeric form to another. This reaction results in the formal migration of a hydrogen atom accompanied by a switch of adjacent conjugated double bonds. In solutions where tautomerization is possible, a chemical equilibrium of the tautomers will be reached. The exact ratio of the tautomers depends on several factors, including temperature, solvent, and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs.

Tautomerizations can be catalyzed by: Base: 1. deprotonation; 2. formation of a delocalized anion (e.g., an enolate); 3. protonation at a different position of the anion; Acid: 1. protonation; 2. formation of a delocalized cation; 3. deprotonation at a different position adjacent to the cation.

The term “analogue” refers to a chemical compound that is structurally similar to another but differs slightly in composition (as in the replacement of one atom by an atom of a different element or in the presence of a particular functional group, or the replacement of one functional group by another functional group). Thus, an analogue is a compound that is similar or comparable in function and appearance, but not in structure or origin to the reference compound.

A “patient,” “subject,” or “host” to be treated by the subject method may mean either a human or non-human animal, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

The terms “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

The term “ED50” is art-recognized. In certain embodiments, ED50 means the dose of a drug, which produces 50% of its maximum response or effect, or alternatively, the dose, which produces a pre-determined response in 50% of test subjects or preparations. The term “LD50” is art-recognized. In certain embodiments, LD50 means the dose of a drug, which is lethal in 50% of test subjects. The term “therapeutic index” is an art-recognized term, which refers to the therapeutic index of a drug, defined as LD50/ED50.

The terms “IC₅₀,” or “half maximal inhibitory concentration” is intended to refer to the concentration of a substance (e.g., a compound or a drug) that is required for 50% inhibition of a biological process, or component of a process, including a protein, subunit, organelle, ribonucleoprotein, etc.

With respect to any chemical compounds, the present application is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

When a bond to a substituent is shown to cross a bond connecting two atoms in a ring, then such substituent can be bonded to any atom in the ring. When a substituent is listed without indicating the atom via which such substituent is bonded to the rest of the compound of a given formula, then such substituent can be bonded via any atom in such substituent. Combinations of substituents and/or variables are permissible, but only if such combinations result in stable compounds.

When an atom or a chemical moiety is followed by a subscripted numeric range (e.g., C₁₋₆), it is meant to encompass each number within the range as well as all intermediate ranges. For example, “C₁₋₆ alkyl” is meant to include alkyl groups with 1, 2, 3, 4, 5, 6, 1-6, 1-5, 1-4, 1-3, 1-2, 2-6, 2-5, 2-4, 2-3, 3-6, 3-5, 3-4, 4-6, 4-5, and 5-6 carbons.

The term “alkyl” is intended to include both branched (e.g., isopropyl, tert-butyl, isobutyl), straight-chain e.g., methyl, ethyl, propyl, butyl, pentyl, hexyl, heptyl, octyl, nonyl, decyl), and cycloalkyl (e.g., alicyclic) groups (e.g., cyclopropyl, cyclopentyl, cyclohexyl, cycloheptyl, cyclooctyl), alkyl substituted cycloalkyl groups, and cycloalkyl substituted alkyl groups. Such aliphatic hydrocarbon groups have a specified number of carbon atoms. For example, C₁₋₆ alkyl is intended to include C₁, C₂, C₃, C₄, C₅, and C₆ alkyl groups. As used herein, “lower alkyl” refers to alkyl groups having from 1 to 6 carbon atoms in the backbone of the carbon chain. “Alkyl” further includes alkyl groups that have oxygen, nitrogen, sulfur or phosphorous atoms replacing one or more hydrocarbon backbone carbon atoms. In certain embodiments, a straight chain or branched chain alkyl has six or fewer carbon atoms in its backbone (e.g., C₁-C₆ for straight chain, C₃-C₆ for branched chain), for example four or fewer. Likewise, certain cycloalkyls have from three to eight carbon atoms in their ring structure, such as five or six carbons in the ring structure.

The term “substituted alkyls” refers to alkyl moieties having substituents replacing a hydrogen on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “aralkyl” moiety is an alkyl substituted with an aryl (e.g., phenylmethyl (benzyl)). If not otherwise indicated, the terms “alkyl” and “lower alkyl” include linear, branched, cyclic, unsubstituted, substituted, and/or heteroatom-containing alkyl or lower alkyl, respectively.

The term “alkenyl” refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, cyclopentenyl, cyclohexenyl, cyclooctenyl, and the like. Generally, although again not necessarily, alkenyl groups can contain 2 to about 18 carbon atoms, and more particularly 2 to 12 carbon atoms. The term “lower alkenyl” refers to an alkenyl group of 2 to 6 carbon atoms, and the specific term “cycloalkenyl” intends a cyclic alkenyl group, preferably having 5 to 8 carbon atoms. The term “substituted alkenyl” refers to alkenyl substituted with one or more substituent groups, and the terms “heteroatom-containing alkenyl” and “heteroalkenyl” refer to alkenyl or heterocycloalkenyl (e.g., heterocylcohexenyl) in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” refers to a linear or branched hydrocarbon group of 2 to 24 carbon atoms containing at least one triple bond, such as ethynyl, n-propynyl, and the like. Generally, although again not necessarily, alkynyl groups can contain 2 to about 18 carbon atoms, and more particularly can contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The terms “alkyl”, “alkenyl”, and “alkynyl” are intended to include moieties which are diradicals, i.e., having two points of attachment. A nonlimiting example of such an alkyl moiety that is a diradical is —CH₂CH₂—, i.e., a C₂ alkyl group that is covalently bonded via each terminal carbon atom to the remainder of the molecule.

The term “alkoxy” refers to an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Preferred substituents identified as “C₁-C₆ alkoxy” or “lower alkoxy” herein contain 1 to 3 carbon atoms, and particularly preferred such substituents contain 1 or 2 carbon atoms (i.e., methoxy and ethoxy).

The term “aryl” refers to an aromatic substituent containing a single aromatic ring or multiple aromatic rings that are fused together, directly linked, or indirectly linked (such that the different aromatic rings are bound to a common group such as a methylene or ethylene moiety). Aryl groups can contain 5 to 20 carbon atoms, and particularly preferred aryl groups can contain 5 to 14 carbon atoms. Examples of aryl groups include benzene, phenyl, pyrrole, furan, thiophene, thiazole, isothiazole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkylamino, dialkylamino, arylamino, diaryl amino, and al kylaryl amino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings, which are not aromatic so as to form a multicyclic system (e.g., tetralin, methylenedioxyphenyl). If not otherwise indicated, the term “aryl” includes unsubstituted, substituted, and/or heteroatom-containing aromatic substituents.

The term “alkaryl” refers to an aryl group with an alkyl substituent, and the term “aralkyl” refers to an alkyl group with an aryl substituent, wherein “aryl” and “alkyl” are as defined above. Exemplary aralkyl groups contain 6 to 24 carbon atoms, and particularly preferred aralkyl groups contain 6 to 16 carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. Alkaryl groups include, for example, p-methylphenyl, 2,4-dimethylphenyl, p-cyclohexylphenyl, 2,7-dimethylnaphthyl, 7-cyclooctylnaphthyl, 3-ethyl-cyclopenta-1,4-diene, and the like.

The terms “heterocyclyl” or “heterocyclic group” include closed ring structures, e.g., 3- to 10-, or 4- to 7-membered rings, which include one or more heteroatoms. “Heteroatom” includes atoms of any element other than carbon or hydrogen. Examples of heteroatoms include nitrogen, oxygen, sulfur and phosphorus.

Heterocyclyl groups can be saturated or unsaturated and include pyrrolidine, oxolane, thiolane, piperidine, piperazine, morpholine, lactones, lactams, such as azetidinones and pyrrolidinones, sultams, and sultones. Heterocyclic groups such as pyrrole and furan can have aromatic character. They include fused ring structures, such as quinoline and isoquinoline. Other examples of heterocyclic groups include pyridine and purine. The heterocyclic ring can be substituted at one or more positions with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety. Heterocyclic groups can also be substituted at one or more constituent atoms with, for example, a lower alkyl, a lower alkenyl, a lower alkoxy, a lower alkylthio, a lower alkylamino, a lower alkylcarboxyl, a nitro, a hydroxyl, —CF₃, or —CN, or the like.

The term “halo” or “halogen” refers to fluoro, chloro, bromo, and iodo. “Counterion” is used to represent a small, negatively charged species such as fluoride, chloride, bromide, iodide, hydroxide, acetate, and sulfate. The term sulfoxide refers to a sulfur attached to 2 different carbon atoms and one oxygen and the S—O bond can be graphically represented with a double bond (S═O), a single bond without charges (S—O) or a single bond with charges [S(+)-O(−)].

The terms “substituted” as in “substituted alkyl,” “substituted aryl,” and the like, as alluded to in some of the aforementioned definitions, is meant that in the alkyl, aryl, or other moiety, at least one hydrogen atom bound to a carbon (or other) atom is replaced with one or more non-hydrogen substituents. Examples of such substituents include, without limitation: functional groups such as halo, hydroxyl, silyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), mono-(C₁-C₂₄ alkyl)-substituted carbamoyl (—(CO)—NH(C₁-C₂₄ alkyl)), di-(C₁-C₄ alkyl)-substituted carbamoyl (—(CO)—N(C₁-C₂₄ alkyl)₂), mono-substituted arylcarbamoyl (—(CO)—NH-aryl), thiocarbamoyl (—(CS)—NH₂), carbamido (—NH—(CO)—NH₂), cyano(—CN), isocyano (—N⁺C⁻), cyanato (—O—CN), isocyanato (—ON⁺C⁻), isothiocyanato (—S—CN), azido (—N═N⁺═N⁻), formyl (—(CO)—H), thioformyl (—(CS)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—(CO)-alkyl), C₆-C₂₀ arylamido (—NH—(CO)-aryl), imino (—CR═NH where R═hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R═hydrogen, alkyl, aryl, alkaryl, etc.), arylimino (—CR═N(aryl), where R═hydrogen, alkyl, aryl, alkaryl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O⁻)₂), phosphinato (—P(O)(O⁻)), phospho (—PO₂), and phosphino (—PH₂); and the hydrocarbyl moieties C₁-C₂₄ alkyl, C₂-C₂₄ alkenyl, C₂-C₂₄ alkynyl, C₅-C₂₀ aryl, C₆-C₂₄ alkaryl, and C₆-C₂₄ aralkyl.

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, substituted alkenyl, and substituted aryl.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

The terms “stable compound” and “stable structure” are meant to indicate a compound that is sufficiently robust to survive isolation, and as appropriate, purification from a reaction mixture, and formulation into an efficacious therapeutic agent.

The terms “free compound” is used herein to describe a compound in the unbound state.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “small molecule” is an art-recognized term. In certain embodiments, this term refers to a molecule, which has a molecular weight of less than about 2000 amu, or less than about 1000 amu, and even less than about 500 amu.

All percentages and ratios used herein, unless otherwise indicated, are by weight.

The term “neoplasm” refers to any abnormal mass of cells or tissue as a result of neoplasia. The neoplasm may be benign, potentially malignant (precancerous), or malignant (cancerous). An adenoma is an example of a neoplasm.

The terms “adenoma”, “colon adenoma” and “polyp” are used herein to describe any precancerous neoplasm of the colon.

The term “colon” as used herein is intended to encompass the right colon (including the cecum), the transverse colon, the left colon and the rectum.

The terms “colorectal cancer” and “colon cancer” are used interchangeably herein to refer to any cancerous neoplasia of the colon (including the rectum, as defined above).

The terms “gene expression” or “protein expression” includes any information pertaining to the amount of gene transcript or protein present in a sample, as well as information about the rate at which genes or proteins are produced or are accumulating or being degraded (e.g., reporter gene data, data from nuclear runoff experiments, pulse-chase data etc.). Certain kinds of data might be viewed as relating to both gene and protein expression. For example, protein levels in a cell are reflective of the level of protein as well as the level of transcription, and such data is intended to be included by the phrase “gene or protein expression information”. Such information may be given in the form of amounts per cell, amounts relative to a control gene or protein, in unitless measures, etc.; the term “information” is not to be limited to any particular means of representation and is intended to mean any representation that provides relevant information. The term “expression levels” refers to a quantity reflected in or derivable from the gene or protein expression data, whether the data is directed to gene transcript accumulation or protein accumulation or protein synthesis rates, etc.

The terms “healthy” and “normal” are used interchangeably herein to refer to a subject or particular cell or tissue that is devoid (at least to the limit of detection) of a disease condition.

The term “nucleic acid” refers to polynucleotides such as deoxyribonucleic acid (DNA), and, where appropriate, ribonucleic acid (RNA). The term should also be understood to include analogues of either RNA or DNA made from nucleotide analogues, and, as applicable to the embodiment being described, single-stranded (such as sense or antisense) and double-stranded polynucleotides. In some embodiments, “nucleic acid” refers to inhibitory nucleic acids. Some categories of inhibitory nucleic acid compounds include antisense nucleic acids, RNAi constructs, and catalytic nucleic acid constructs. Such categories of nucleic acids are well-known in the art.

Embodiments described herein relate to compounds and methods of modulating SCD activity (e.g., 15-PGDH activity), modulating tissue prostaglandin levels, and/or treating diseases, disorders, or conditions in which it is desired to modulate 15-PGDH activity and/or prostaglandin levels.

“Inhibitors,” “activators,” and “modulators” of 15-PGDH expression or of 15-PGDH activity are used to refer to inhibitory, activating, or modulating molecules, respectively, identified using in vitro and in vivo assays for 15-PGDH expression or 15-PGDH activity, e.g., ligands, agonists, antagonists, and their homologs and mimetics. The term “modulator” includes inhibitors and activators. Inhibitors are agents that, e.g., inhibit expression of 15-PGDH or bind to, partially or totally block stimulation, decrease, prevent, delay activation, inactivate, desensitize, or down regulate the activity of 15-PGDH, e.g., antagonists. Activators are agents that, e.g., induce or activate the expression of a 15-PGDH or bind to, stimulate, stabilize, increase, open, activate, facilitate, or enhance activation, sensitize or up regulate the activity of 15-PGDH, e.g., agonists. Modulators include naturally occurring and synthetic ligands, small chemical molecules, and the like.

15-PGDH inhibitors described herein can provide a pharmacologic method for elevating prostaglandin levels in tissue. Known activities of prostaglandins include promoting hair growth, promoting skin pigmentation, and promoting skin darkening or the appearance of skin tanning. Known activities of prostaglandins also include ameliorating pulmonary artery hypertension. 15-PGDH inhibitors described herein may also be utilized to increase tissue stem cell numbers for purposes that would include increasing resistance to tissue damage by radiation, increasing resistance to environmental exposures to radiation, increasing stem cell numbers to increase fitness of bone marrow or other types of transplantation (through either in vivo exposure to 15-PGDH inhibitors described herein to increase stem cell numbers prior to harvest of a transplanted tissue, or through ex vivo exposure of a harvested tissue prior to transplant into a recipient host, or through treatment of the graft recipient). 15-PGDH inhibitors described herein may also be utilized for purposes that would include promoting liver regeneration, including liver regeneration after liver resection, and liver regeneration after toxic insults, which for example may be the toxic insult of acetaminophen overdose. Prostaglandin signaling is also known to promote wound healing, protect the stomach from ulceration, and promote healing of ulcers of stomach and intestines. Additionally, 15-PGDH inhibitors described herein can promote activity of human keratinocytes in “healing” scratches across cultures of keratinocyte cells. Hence, 15-PGDH inhibitors described herein may be utilized to also heal ulcers of other tissues, including, but not limited to skin, and including but not limited to diabetic ulcers. Further, 15-PGDH inhibitors described herein may be utilized for the treatment of erectile dysfunction.

15-PGDH inhibitors described herein can be identified using assays in which putative modulator compounds are applied to cells expressing 15-PGDH and then the functional effects on 15-PGDH activity are determined. Samples or assays comprising 15-PGDH that are treated with a potential activator, inhibitor, or modulator are compared to control samples without the inhibitor, activator, or modulator to examine the extent of effect. Control samples (untreated with modulators) are assigned a relative 15-PGDH activity value of 100%. Inhibition of 15-PGDH is achieved when the 15-PGDH activity value relative to the control is about 80%, optionally 50% or 25%, 10%, 5% or 1%.

Agents tested as modulators of SCD (e.g., 15-PGDH) can be any small chemical molecule or compound. Typically, test compounds will be small chemical molecules, natural products, or peptides. The assays are designed to screen large chemical libraries by automating the assay steps and providing compounds from any convenient source to assays, which are typically run in parallel (e.g., in microtiter formats on microtiter plates in robotic assays). Modulators also include agents designed to increase the level of 15-PGDH mRNA or the level of translation from an mRNA.

In some embodiments, the modulator of SCD can be an SCD inhibitor that can be administered to tissue or blood of a subject at an amount effective to inhibit the activity of a short chain dehydrogenase enzyme. The SCD inhibitor can be a 15-PGDH inhibitor that can be administered to tissue or blood of a subject at an amount effective to increase prostaglandin levels in the tissue or blood. The 15-PGDH inhibitor can include a compound having the formula (I):

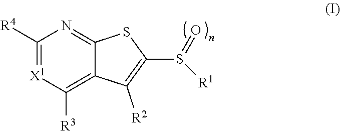

wherein n=0-2;
X¹ is N or CR^{a};
R¹ is selected from the group consisting of branched or linear alkyl including —(CH₂)n₁CH₃ (n₁=0-7),

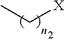

wherein n₂=0-6 and X is any of the following:

CF_{y}H_{z} (y+z=3), CCl_{y}H_{z} (y+z=3), OH, OAc OMe, R⁷¹, OR⁷², CN, N(R⁷³)₂,

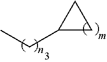

(n₃=0-5, m=1-5), and

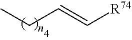

(n₄=0-5);
R² is selected from the group consisting of H, linear or branched alkyl, OH, Cl, F, NH₂, N(R⁷⁶)₂, and OR⁷⁷;
R³ and R⁴ are each independently selected from the group consisting of:

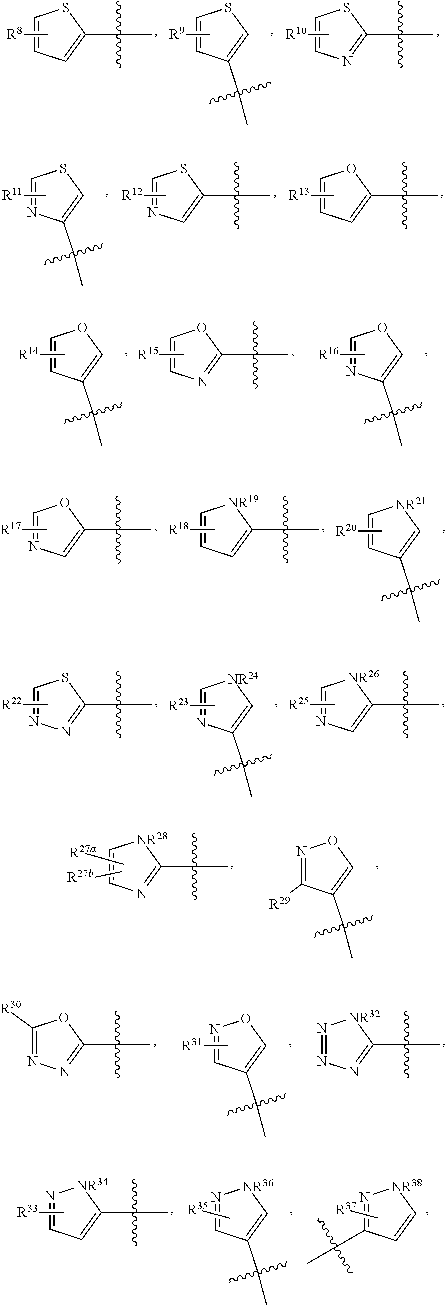

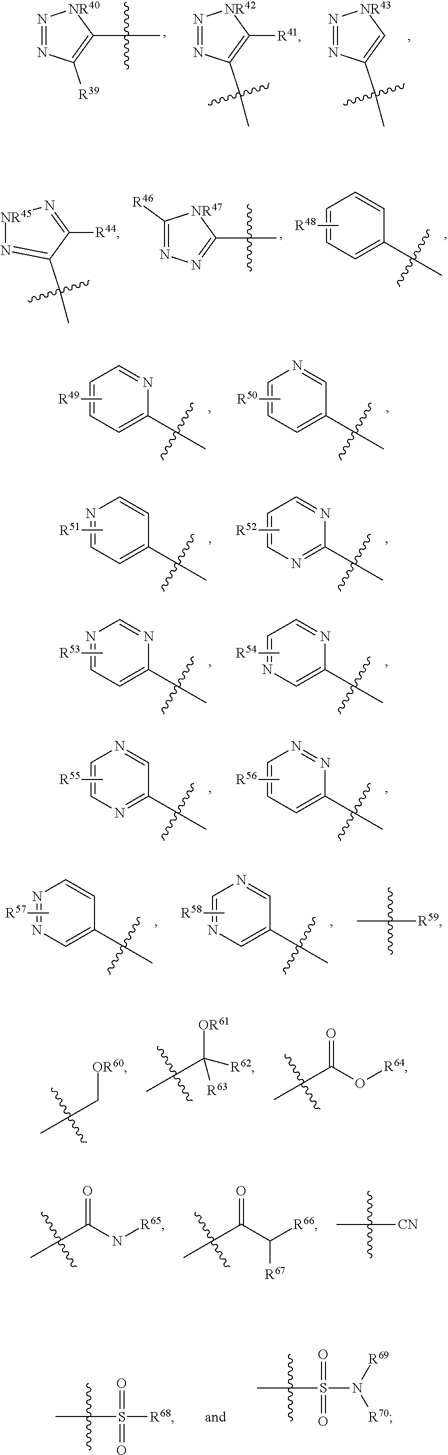

each R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁶, R⁷⁷, and R^{a} are the same or different and are independently selected from the group consisting of hydrogen, substituted or unsubstituted C₁-C₂₄ alkyl, C₂-C₂₄ alkenyl, C₂-C₂₄ alkynyl, C₆-C₂n aryl, heterocycloalkenyl containing from 4-6 ring atoms, (wherein from 1-3 of the ring atoms is independently selected from N, NH, N(C₁-C₆ alkyl), NC(O) (C₁-C₆ alkyl), O, and S), heteroaryl or heterocyclyl containing from 4-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano(—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R═hydrogen, alkyl, aryl, alkaryl, aralkyl, etc.), arylimino (—CR═N(aryl), where R═hydrogen, alkyl, aryl, alkaryl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, arlyl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R═H, methyl or other alkyl], groups incorporating amino acids or other moieties expected to bear positive or negative charge at physiological pH, and combinations thereof; wherein R² and R³ can be linked to form a cyclic or heterocyclic ring, and pharmaceutically acceptable salts thereof.

In some embodiments, X⁶ can be N or CH. R⁴ can be a substituted or unsubstituted heterocyclyl containing 5-6 ring atoms. For example, R⁴ can be a substituted or unsubstituted thiophene, thiazole, oxazole, imidazole, pyridine, or phenyl. R⁷ can be selected from the group consisting of H, substituted or unsubstituted aryl, a substituted or unsubstituted cycloalkyl, and a substituted or unsubstituted heterocyclyl, alkyl, or carboxy including carboxylic acid (—CO₂H), carboxy ester (—CO₂alkyl) and carboxamide [—CON(H)(alkyl) or —CO₂N(alkyl)₂].

In some embodiments, X¹ can be N or CH. R⁴ can be a substituted or unsubstituted heterocyclyl containing 5-6 ring atoms. For example, R⁴ can be a substituted or unsubstituted thiophene, thiazole, oxazole, imidazole, pyridine, or phenyl.

In other embodiments, R⁴ can be selected from the group consisting of:

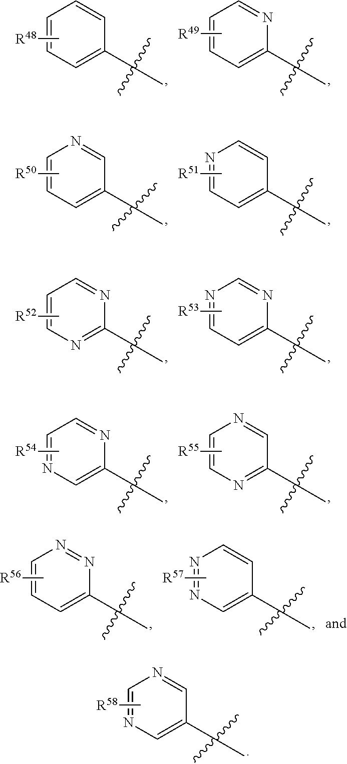

In still other embodiments, R³ is selected from the group consisting of H, substituted or unsubstituted aryl, a substituted or unsubstituted cycloalkyl, and a substituted or unsubstituted heterocyclyl, alkyl, or carboxy including carboxylic acid (—CO₂H), carboxy ester (—CO₂alkyl) and carboxamide [—CON(H)(alkyl) or —CO₂N(alkyl)₂].

Examples of compounds having formula (I) are selected from the group consisting of:

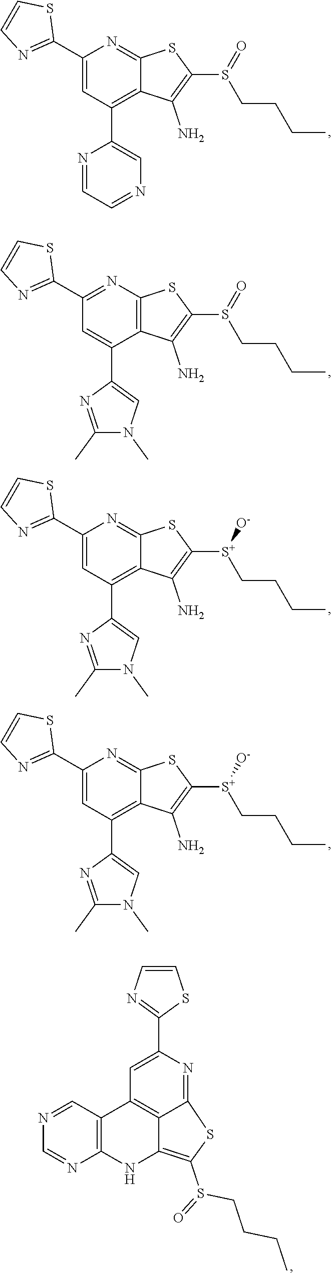

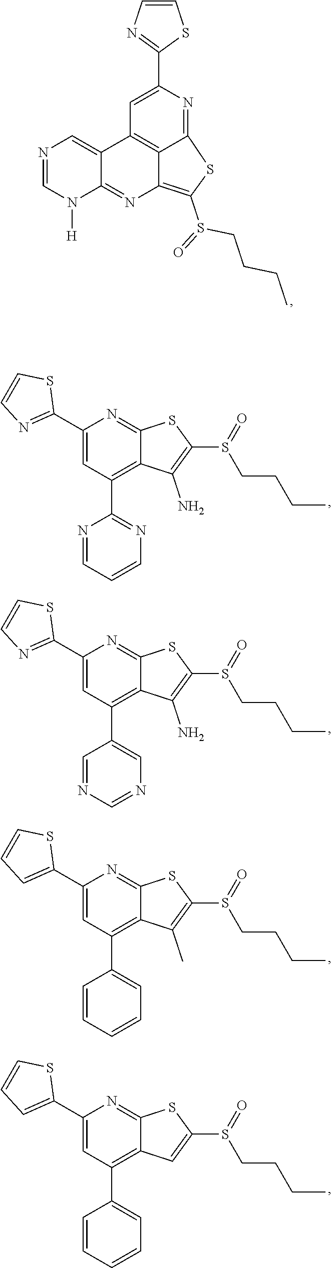 [SMILES: CCCCS(=O)c1sc2nc(-c3nccs3)cc3c4c(nc1c23)NC=NC=4 ; CCCCS(=O)c1sc2nc(-c3nccs3)cc(-c3ncccn3)c2c1N ; CCCCS(=O)c1sc2nc(-c3nccs3)cc(-c3cncnc3)c2c1N ; CCCCS(=O)c1sc2nc(-c3cccs3)cc(-c3ccccc3)c2c1C ; CCCCS(=O)c1cc2c(-c3ccccc3)cc(-c3cccs3)nc2s1]

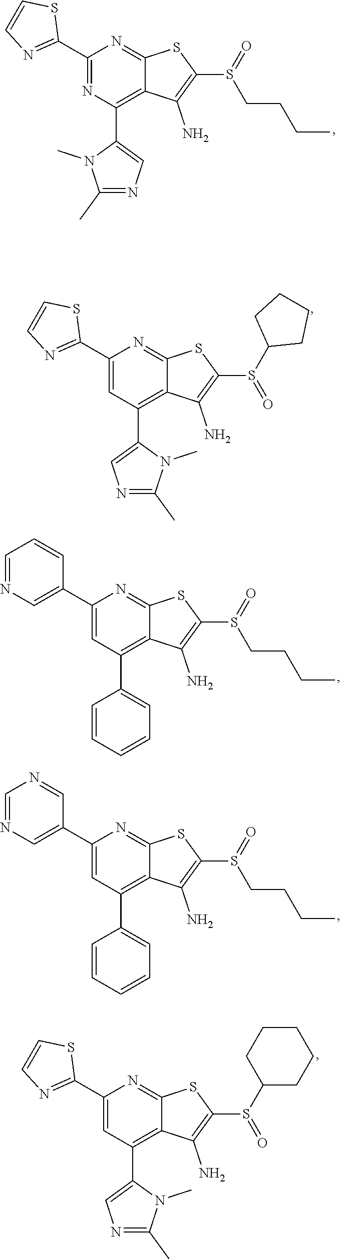 [SMILES: CCCCS(=O)c1sc2nc(-c3nccs3)nc(-c3cnc(C)n3C)c2c1N ; Cc1ncc(-c2cc(-c3nccs3)nc3sc(S(=O)C4CCCC4)c(N)c23)n1C ; CCCCS(=O)c1sc2nc(-c3cccnc3)cc(-c3ccccc3)c2c1N ; CCCCS(=O)c1sc2nc(-c3cncnc3)cc(-c3ccccc3)c2c1N ; Cc1ncc(-c2cc(-c3nccs3)nc3sc(S(=O)C4CCCCC4)c(N)c23)n1C]

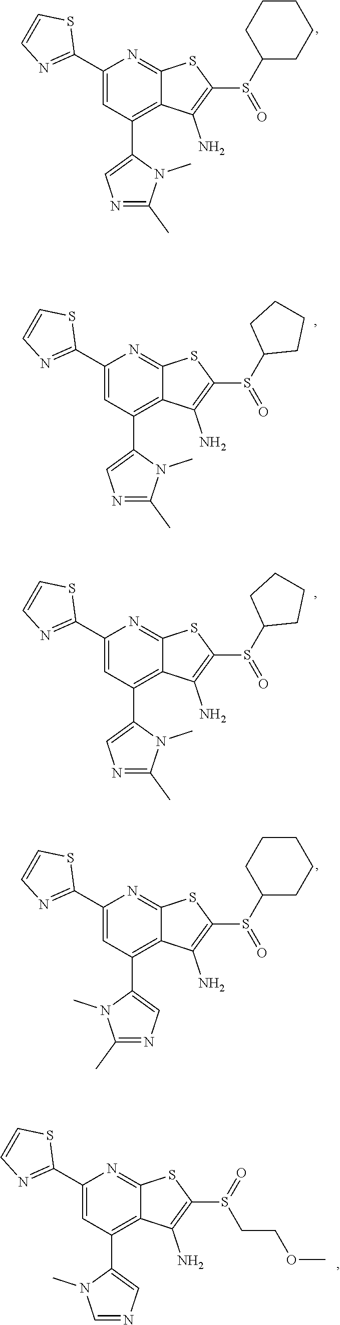 [SMILES: Cc1ncc(-c2cc(-c3nccs3)nc3sc(S(=O)C4CCCCC4)c(N)c23)n1C ; Cc1ncc(-c2cc(-c3nccs3)nc3sc(S(=O)C4CCCC4)c(N)c23)n1C ; Cc1ncc(-c2cc(-c3nccs3)nc3sc(S(=O)C4CCCC4)c(N)c23)n1C ; Cc1ncc(-c2cc(-c3nccs3)nc3sc(S(=O)C4CCCCC4)c(N)c23)n1C ; COCCS(=O)c1sc2nc(-c3nccs3)cc(-c3cncn3C)c2c1N]

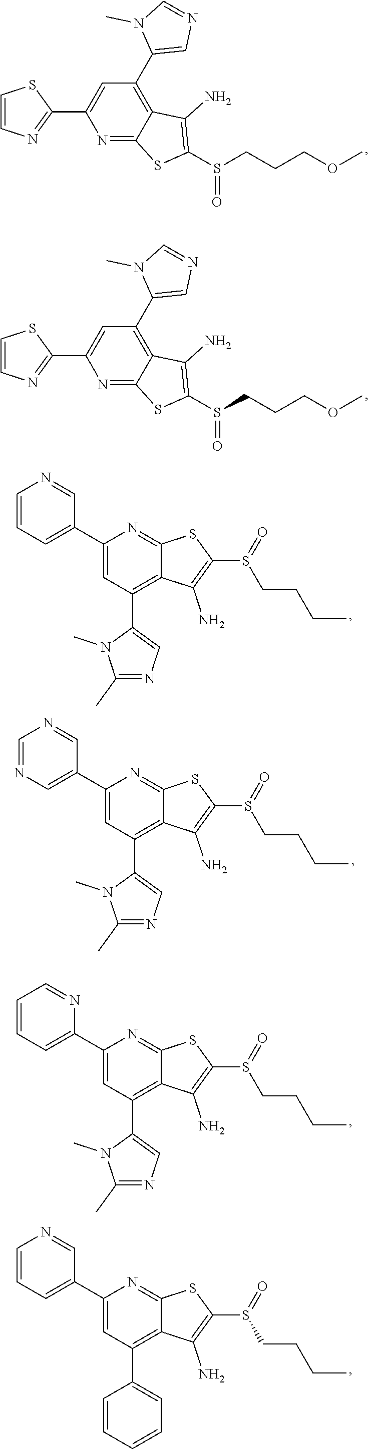

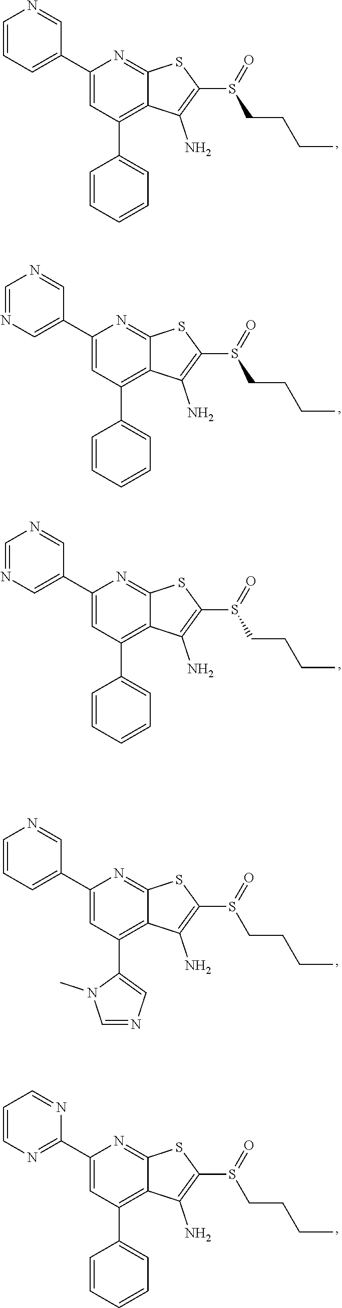 [SMILES: CCCC[S@](=O)c1sc2nc(-c3cccnc3)cc(-c3ccccc3)c2c1N ; CCCC[S@](=O)c1sc2nc(-c3cncnc3)cc(-c3ccccc3)c2c1N ; CCCC[S@@](=O)c1sc2nc(-c3cncnc3)cc(-c3ccccc3)c2c1N ; CCCCS(=O)c1sc2nc(-c3cccnc3)cc(-c3cncn3C)c2c1N ; CCCCS(=O)c1sc2nc(-c3ncccn3)cc(-c3ccccc3)c2c1N]

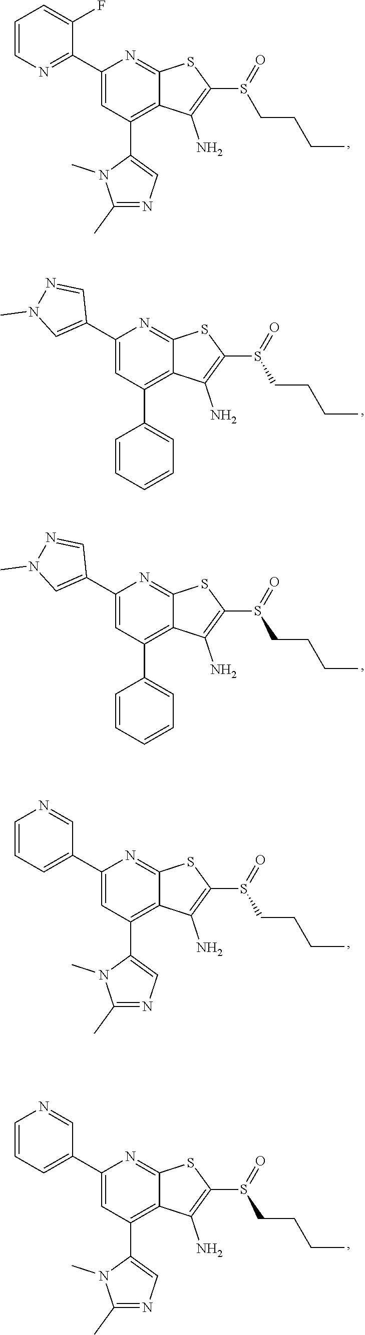

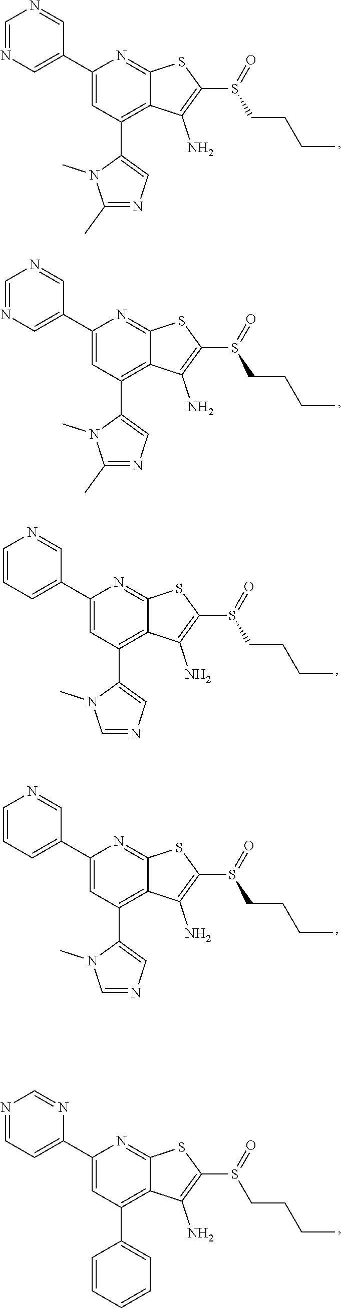 [SMILES: CCCC[S@@](=O)c1sc2nc(-c3cncnc3)cc(-c3cnc(C)n3C)c2c1N ; CCCC[S@](=O)c1sc2nc(-c3cncnc3)cc(-c3cnc(C)n3C)c2c1N ; CCCC[S@@](=O)c1sc2nc(-c3cccnc3)cc(-c3cncn3C)c2c1N ; CCCC[S@](=O)c1sc2nc(-c3cccnc3)cc(-c3cncn3C)c2c1N ; CCCCS(=O)c1sc2nc(-c3ccncn3)cc(-c3ccccc3)c2c1N]

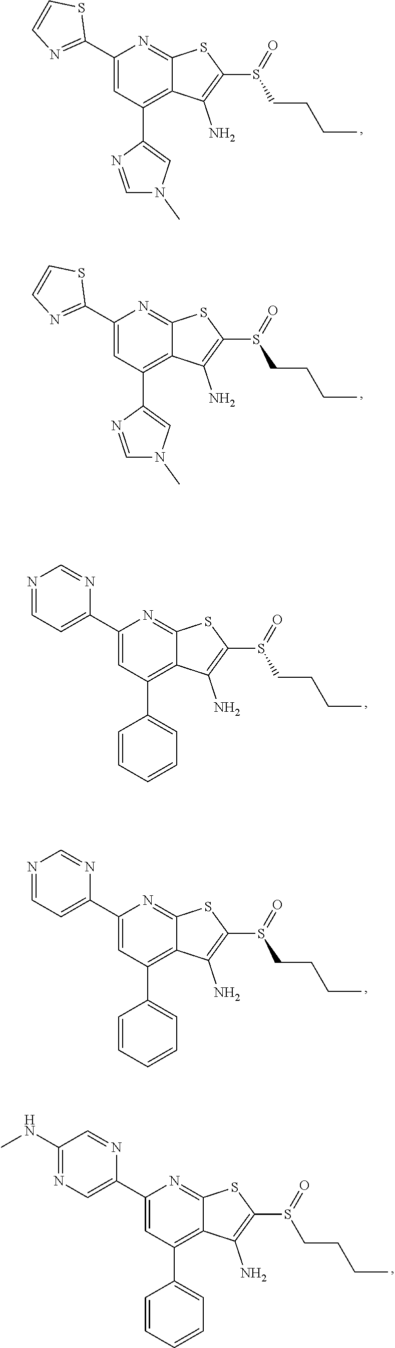

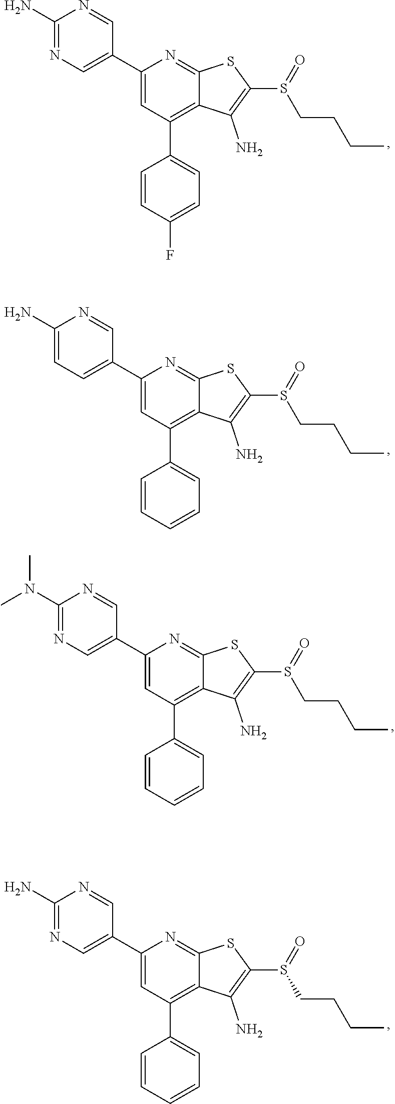

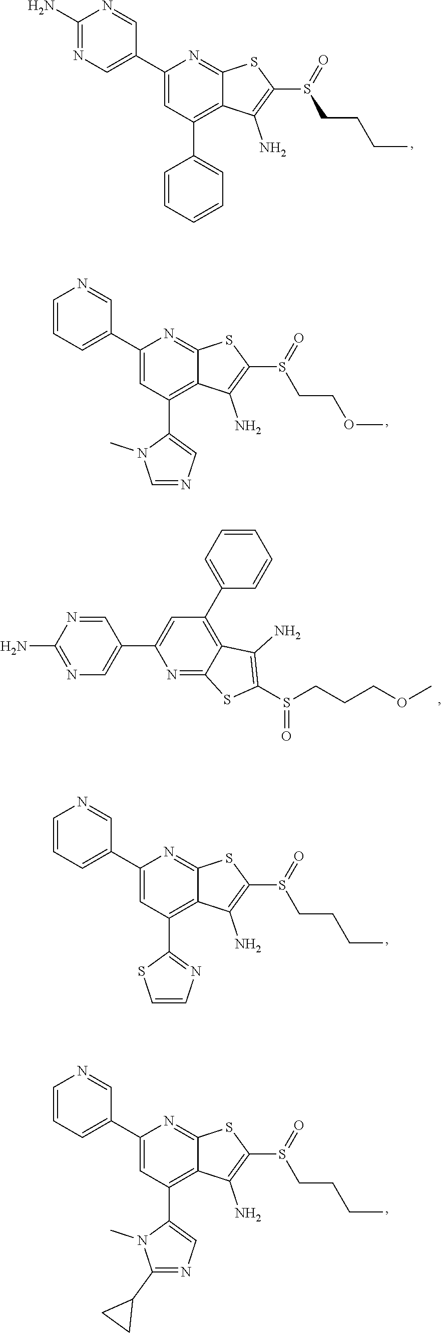 [SMILES: CCCC[S@](=O)c1sc2nc(-c3cnc(N)nc3)cc(-c3ccccc3)c2c1N ; COCCS(=O)c1sc2nc(-c3cccnc3)cc(-c3cncn3C)c2c1N ; COCCCS(=O)c1sc2nc(-c3cnc(N)nc3)cc(-c3ccccc3)c2c1N ; CCCCS(=O)c1sc2nc(-c3cccnc3)cc(-c3nccs3)c2c1N ; CCCCS(=O)c1sc2nc(-c3cccnc3)cc(-c3cnc(C4CC4)n3C)c2c1N]

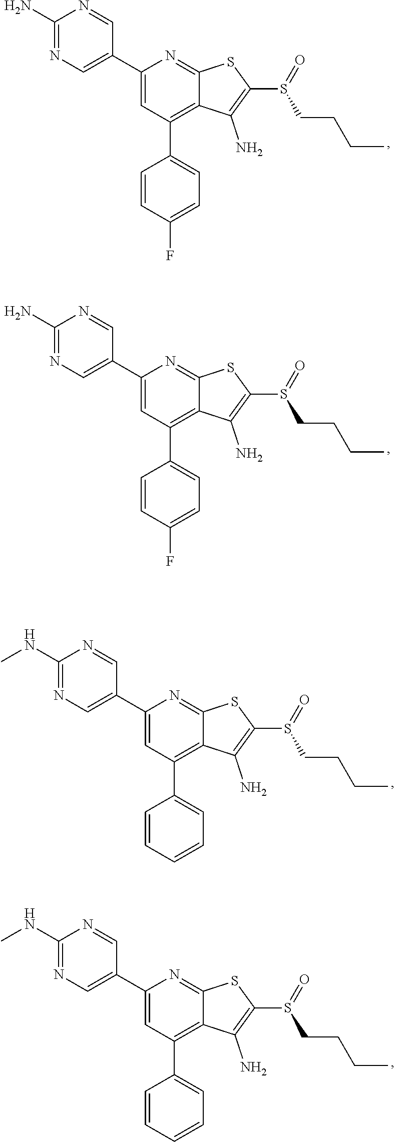

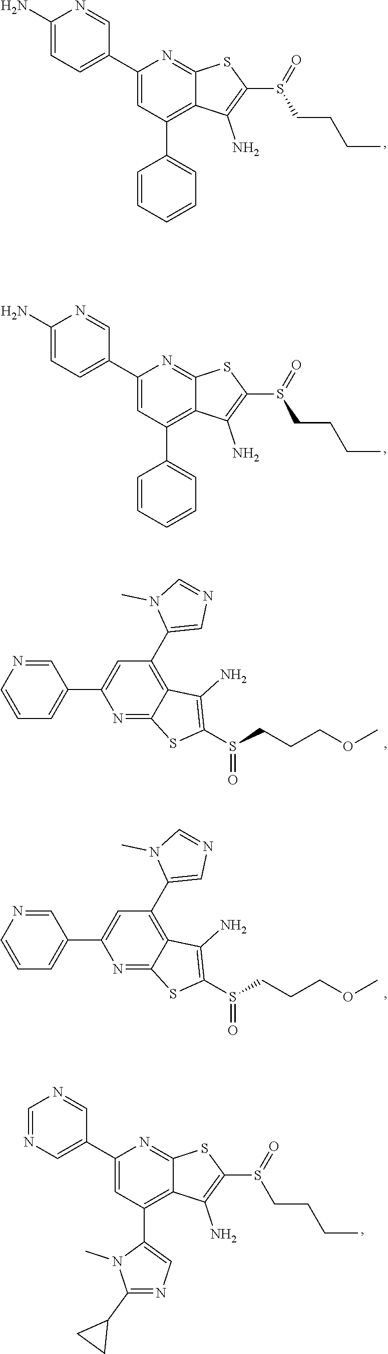 [SMILES: CCCC[S@@](=O)c1sc2nc(-c3ccc(N)nc3)cc(-c3ccccc3)c2c1N ; CCCC[S@](=O)c1sc2nc(-c3ccc(N)nc3)cc(-c3ccccc3)c2c1N ; COCCC[S@@](=O)c1sc2nc(-c3cccnc3)cc(-c3cncn3C)c2c1N ; COCCC[S@](=O)c1sc2nc(-c3cccnc3)cc(-c3cncn3C)c2c1N ; CCCCS(=O)c1sc2nc(-c3cncnc3)cc(-c3cnc(C4CC4)n3C)c2c1N]

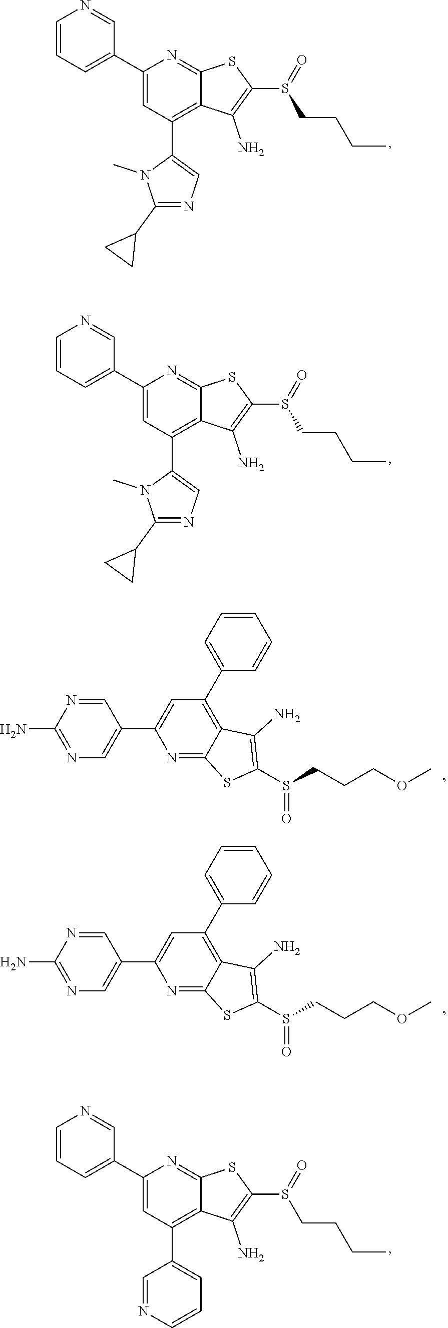

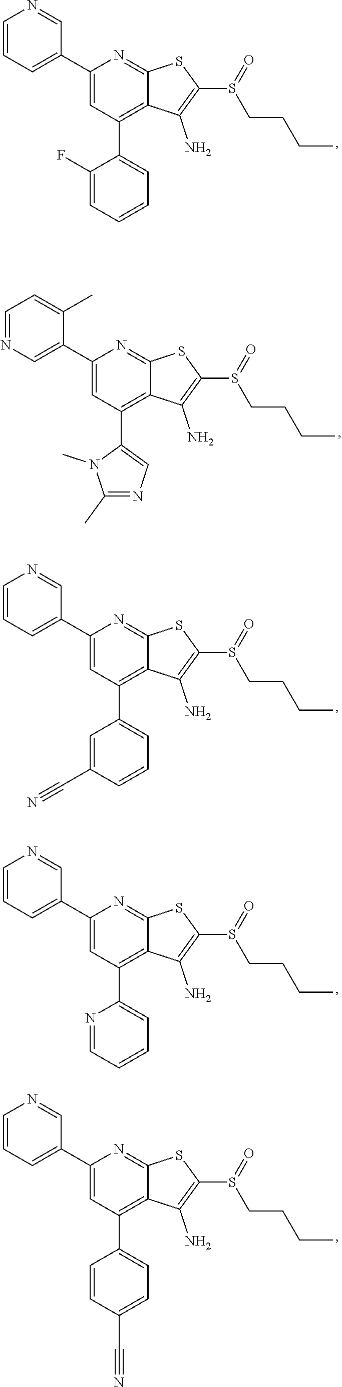 [SMILES: CCCCS(=O)c1sc2nc(-c3cccnc3)cc(-c3ccccc3F)c2c1N ; CCCCS(=O)c1sc2nc(-c3cnccc3C)cc(-c3cnc(C)n3C)c2c1N ; CCCCS(=O)c1sc2nc(-c3cccnc3)cc(-c3cccc(C#N)c3)c2c1N ; CCCCS(=O)c1sc2nc(-c3cccnc3)cc(-c3ccccn3)c2c1N ; CCCCS(=O)c1sc2nc(-c3cccnc3)cc(-c3ccc(C#N)cc3)c2c1N]

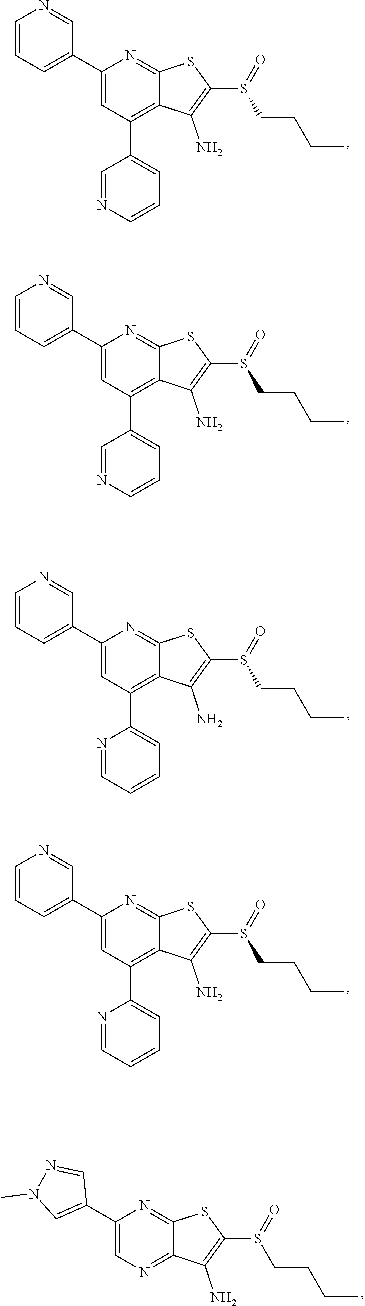

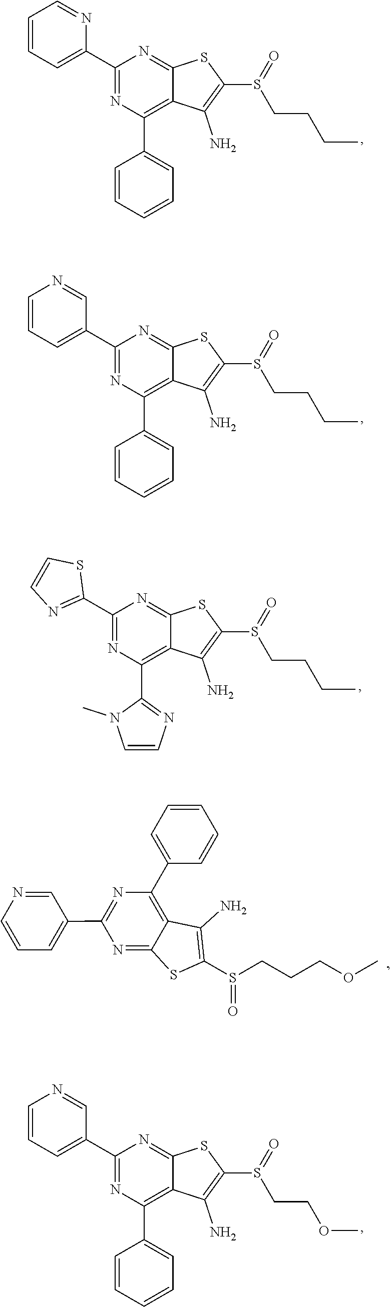 [SMILES: CCCCS(=O)c1sc2nc(-c3ccccn3)nc(-c3ccccc3)c2c1N ; CCCCS(=O)c1sc2nc(-c3cccnc3)nc(-c3ccccc3)c2c1N ; CCCCS(=O)c1sc2nc(-c3nccs3)nc(-c3nccn3C)c2c1N ; COCCCS(=O)c1sc2nc(-c3cccnc3)nc(-c3ccccc3)c2c1N ; COCCS(=O)c1sc2nc(-c3cccnc3)nc(-c3ccccc3)c2c1N]

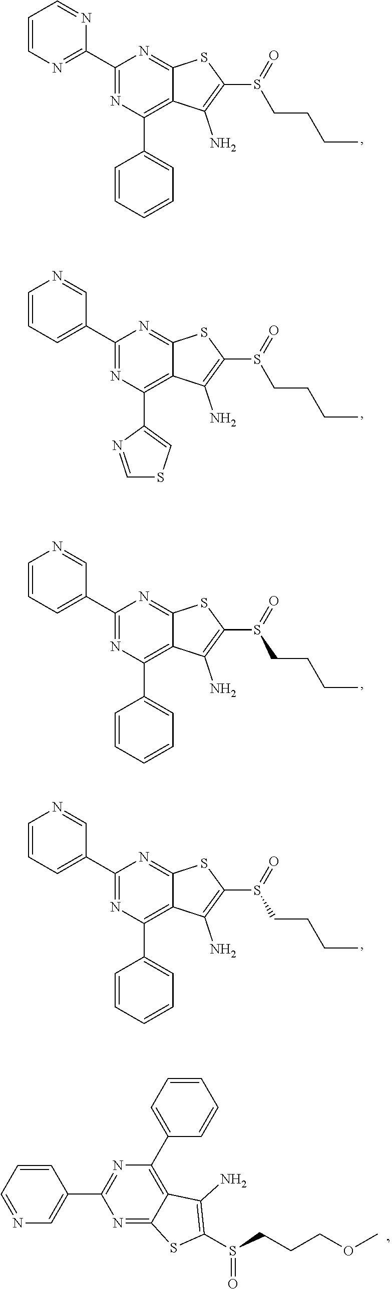 [SMILES: CCCCS(=O)c1sc2nc(-c3ncccn3)nc(-c3ccccc3)c2c1N ; CCCCS(=O)c1sc2nc(-c3cccnc3)nc(-c3cscn3)c2c1N ; CCCC[S@](=O)c1sc2nc(-c3cccnc3)nc(-c3ccccc3)c2c1N ; CCCC[S@@](=O)c1sc2nc(-c3cccnc3)nc(-c3ccccc3)c2c1N ; COCCC[S@@](=O)c1sc2nc(-c3cccnc3)nc(-c3ccccc3)c2c1N]

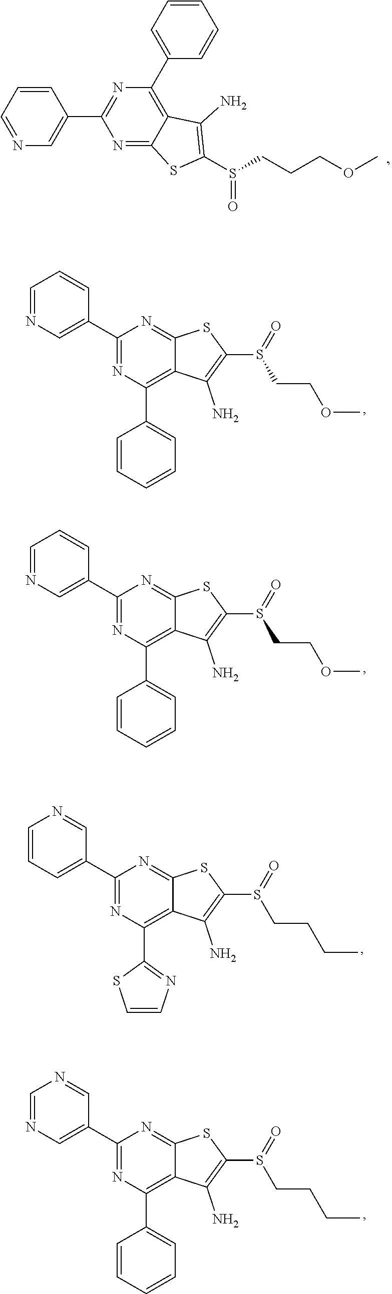 [SMILES: COCCC[S@](=O)c1sc2nc(-c3cccnc3)nc(-c3ccccc3)c2c1N ; COCC[S@@](=O)c1sc2nc(-c3cccnc3)nc(-c3ccccc3)c2c1N ; COCC[S@](=O)c1sc2nc(-c3cccnc3)nc(-c3ccccc3)c2c1N ; CCCCS(=O)c1sc2nc(-c3cccnc3)nc(-c3nccs3)c2c1N ; CCCCS(=O)c1sc2nc(-c3cncnc3)nc(-c3ccccc3)c2c1N]

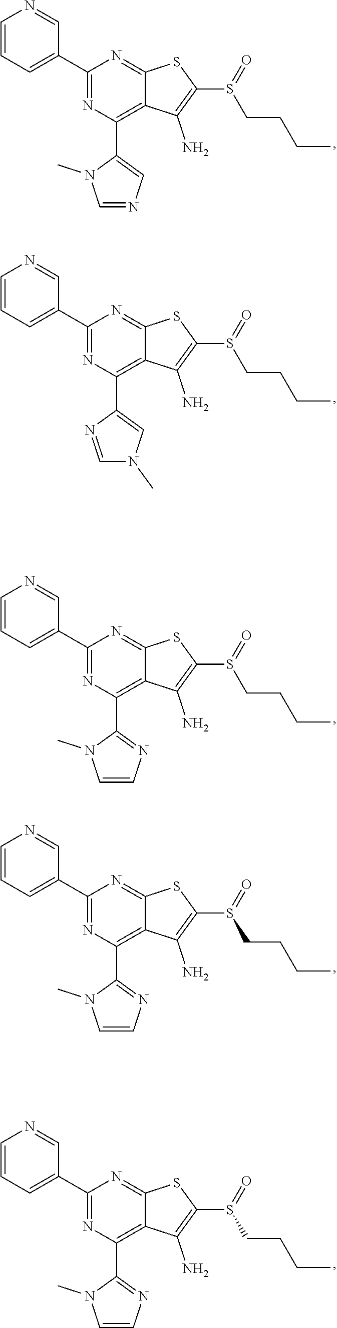 [SMILES: CCCCS(=O)c1sc2nc(-c3cccnc3)nc(-c3cncn3C)c2c1N ; CCCCS(=O)c1sc2nc(-c3cccnc3)nc(-c3cn(C)cn3)c2c1N ; CCCCS(=O)c1sc2nc(-c3cccnc3)nc(-c3nccn3C)c2c1N ; CCCC[S@](=O)c1sc2nc(-c3cccnc3)nc(-c3nccn3C)c2c1N ; CCCC[S@@](=O)c1sc2nc(-c3cccnc3)nc(-c3nccn3C)c2c1N]

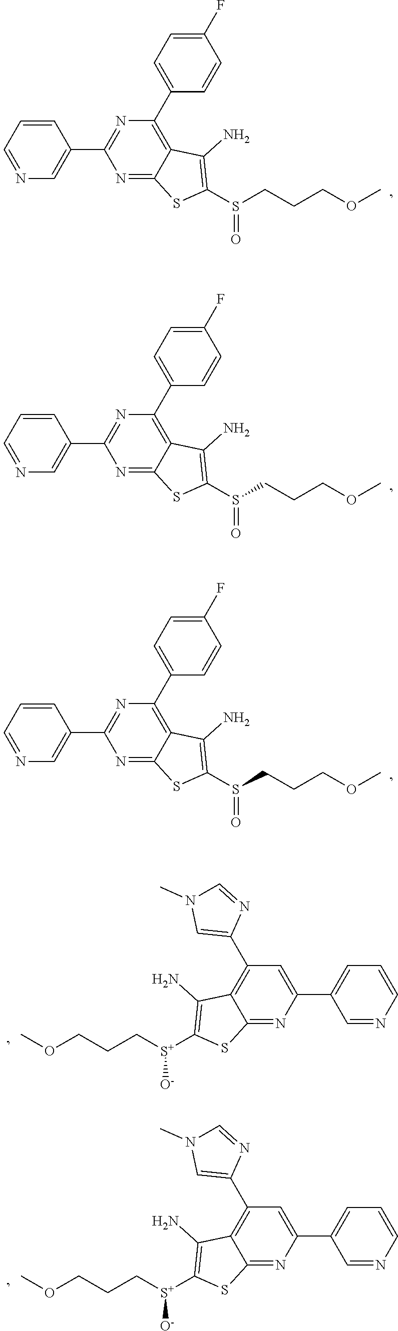

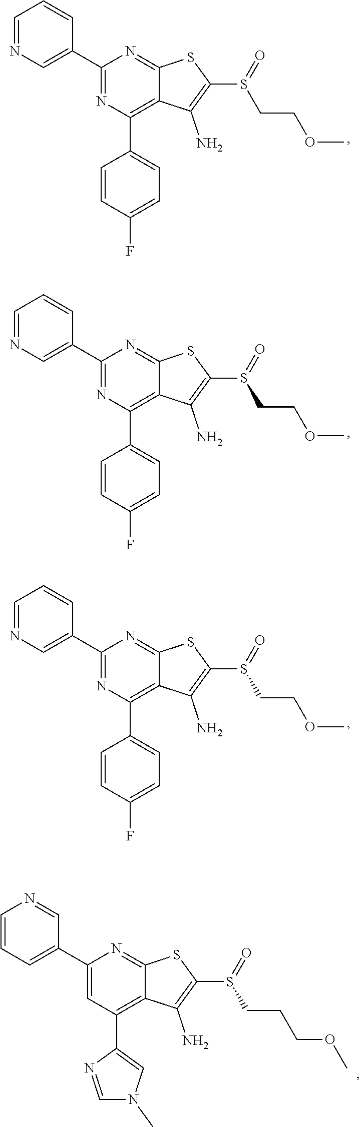 [SMILES: COCCS(=O)c1sc2nc(-c3cccnc3)nc(-c3ccc(F)cc3)c2c1N ; COCC[S@](=O)c1sc2nc(-c3cccnc3)nc(-c3ccc(F)cc3)c2c1N ; COCC[S@@](=O)c1sc2nc(-c3cccnc3)nc(-c3ccc(F)cc3)c2c1N ; COCCC[S@@](=O)c1sc2nc(-c3cccnc3)cc(-c3cn(C)cn3)c2c1N]

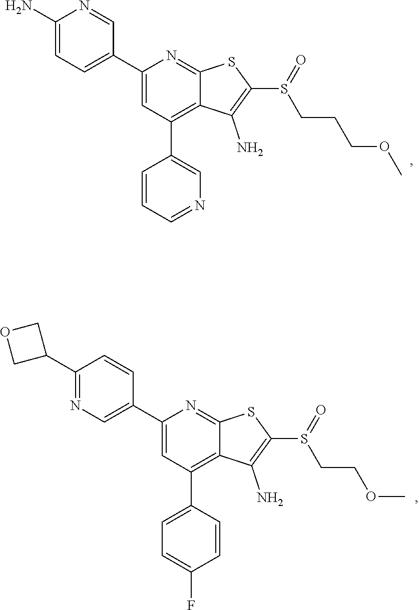 [SMILES: COCCCS(=O)c1sc2nc(-c3ccc(N)nc3)cc(-c3cccnc3)c2c1N ; COCCS(=O)c1sc2nc(-c3ccc(C4COC4)nc3)cc(-c3ccc(F)cc3)c2c1N]

and pharmaceutically acceptable salts thereof.

In some embodiments, the 15-PGDH inhibitor having formula (I) is not a compound having the following formula:

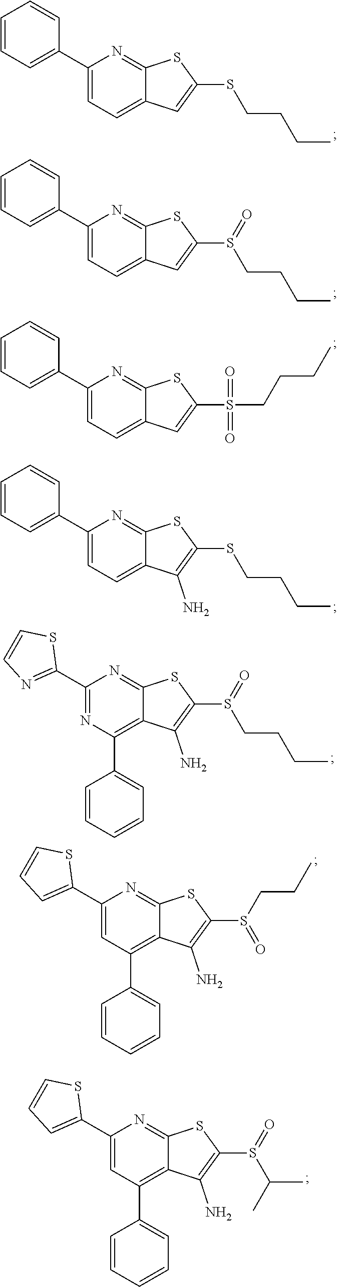

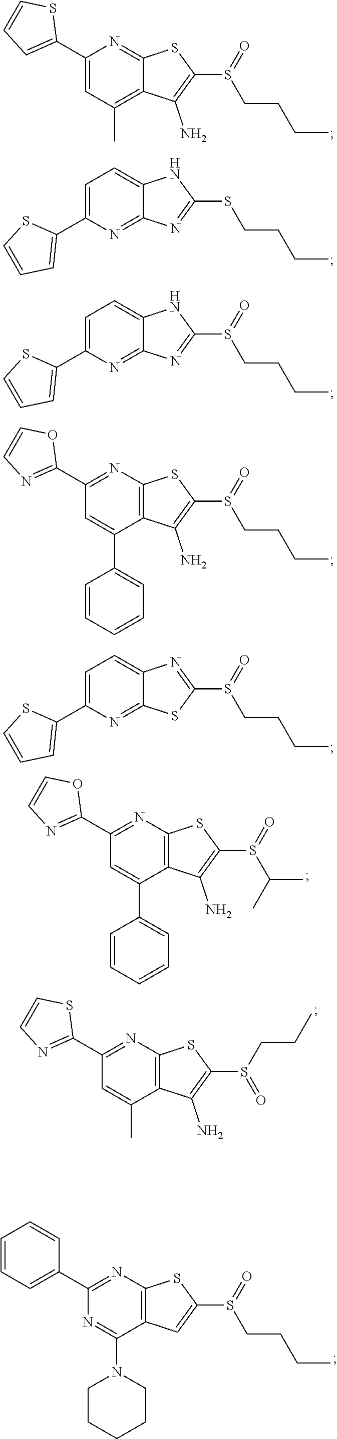

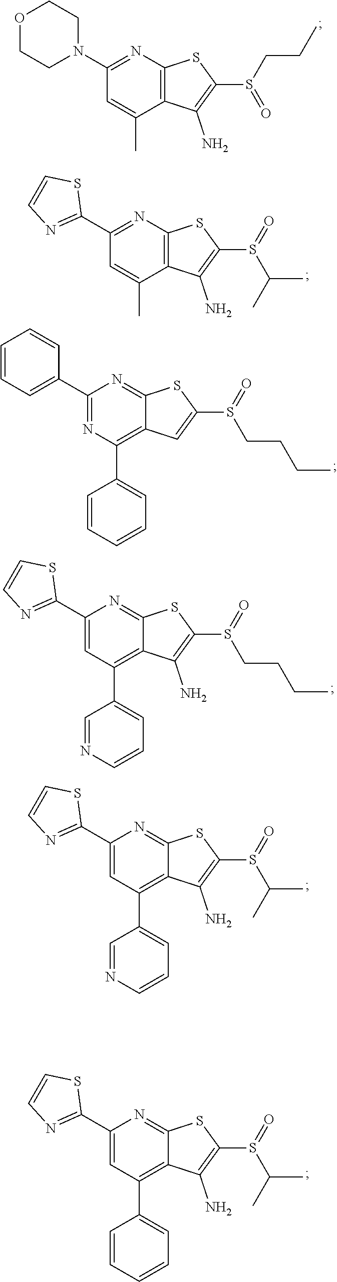

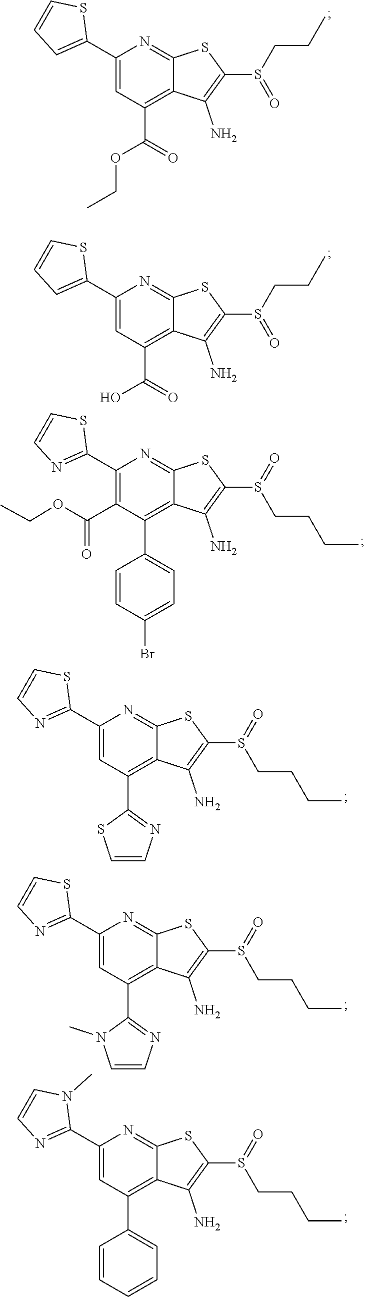

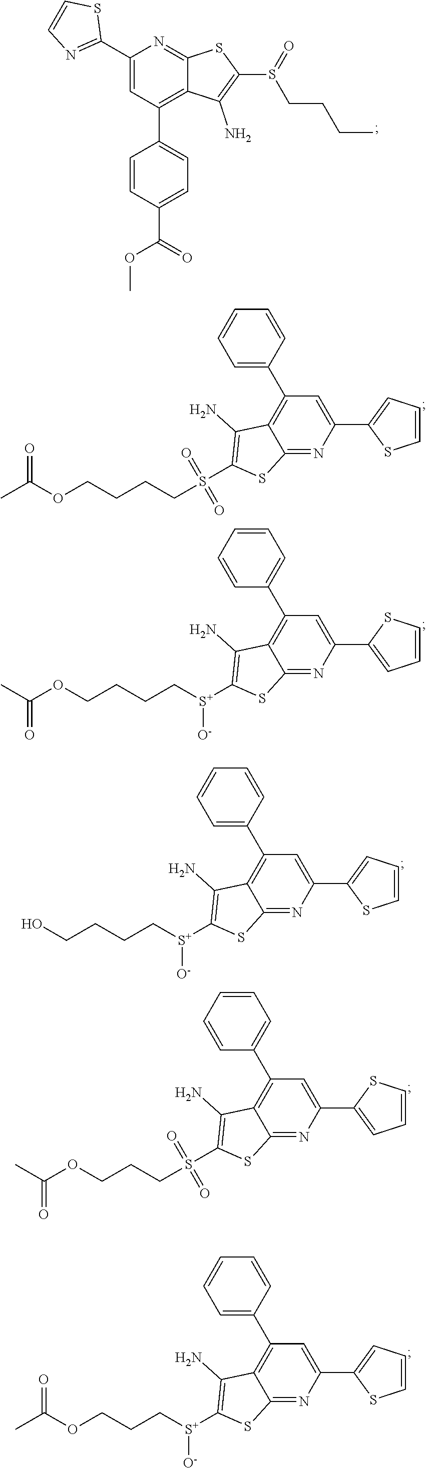

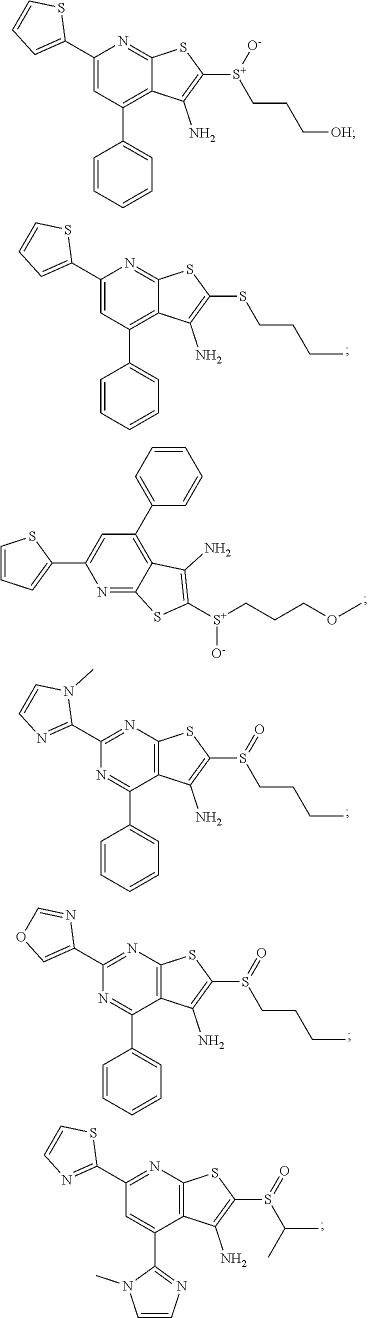

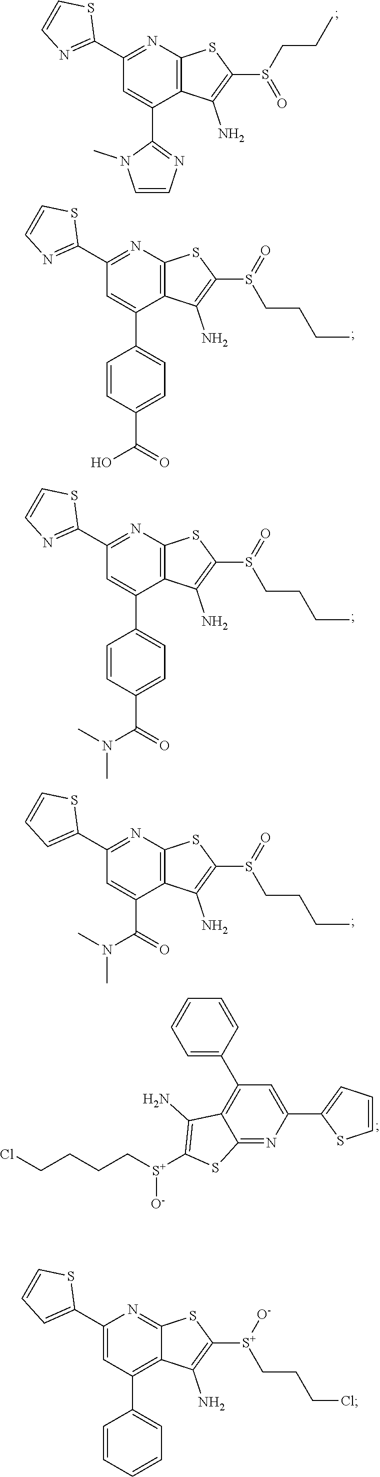

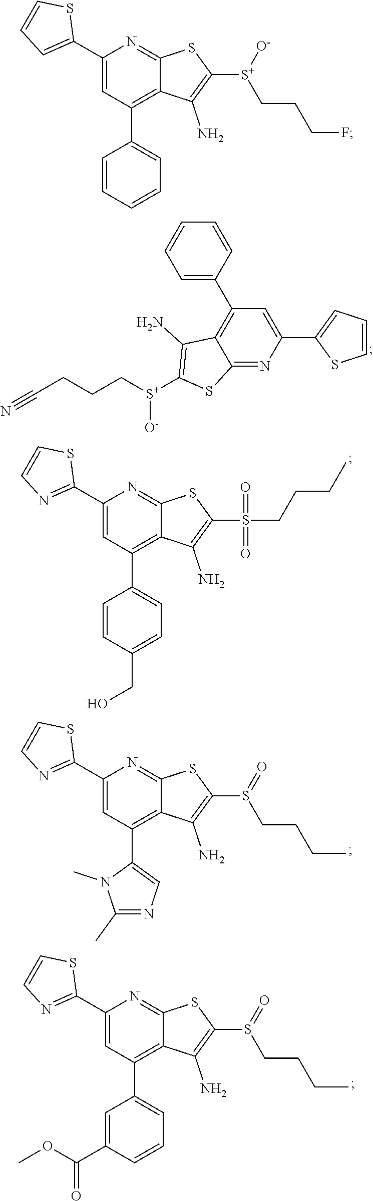

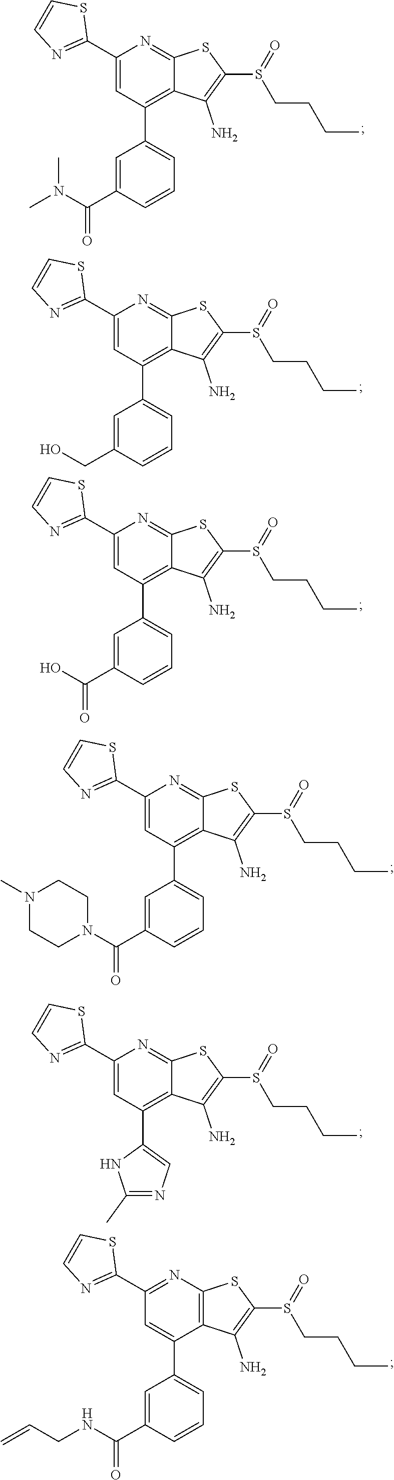

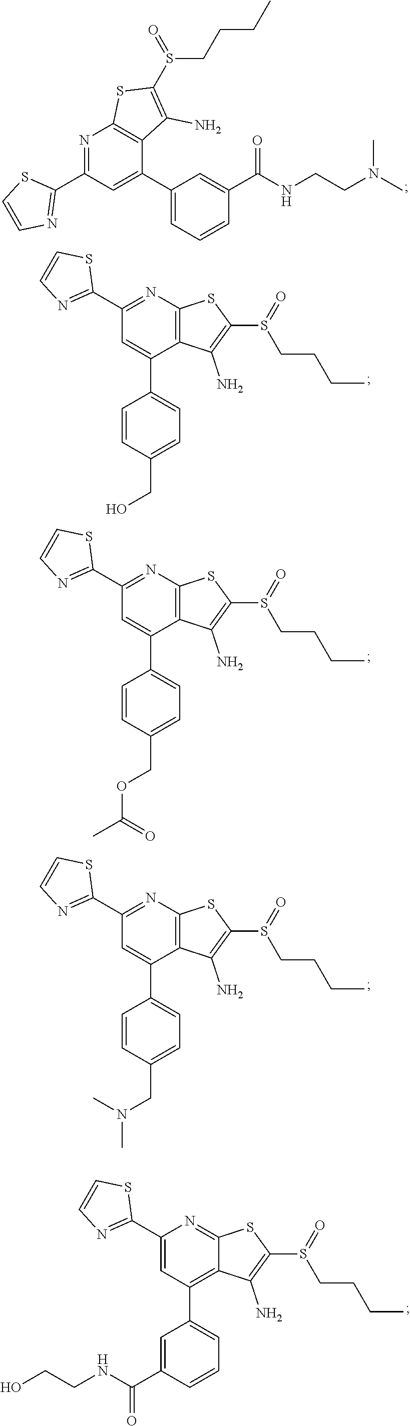

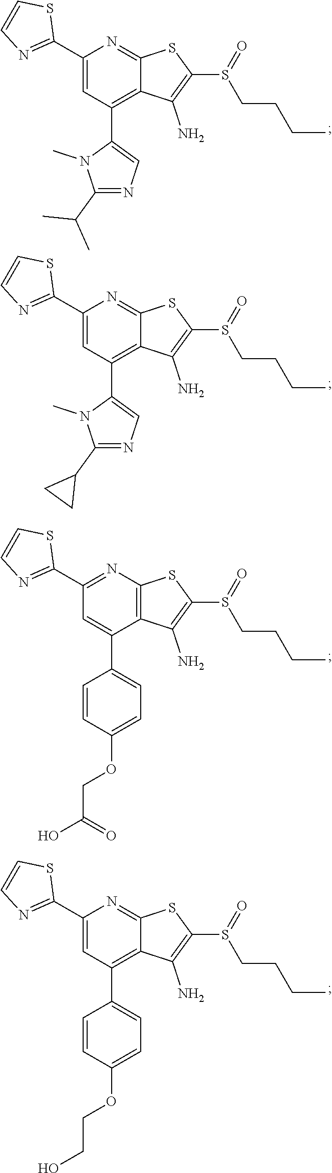

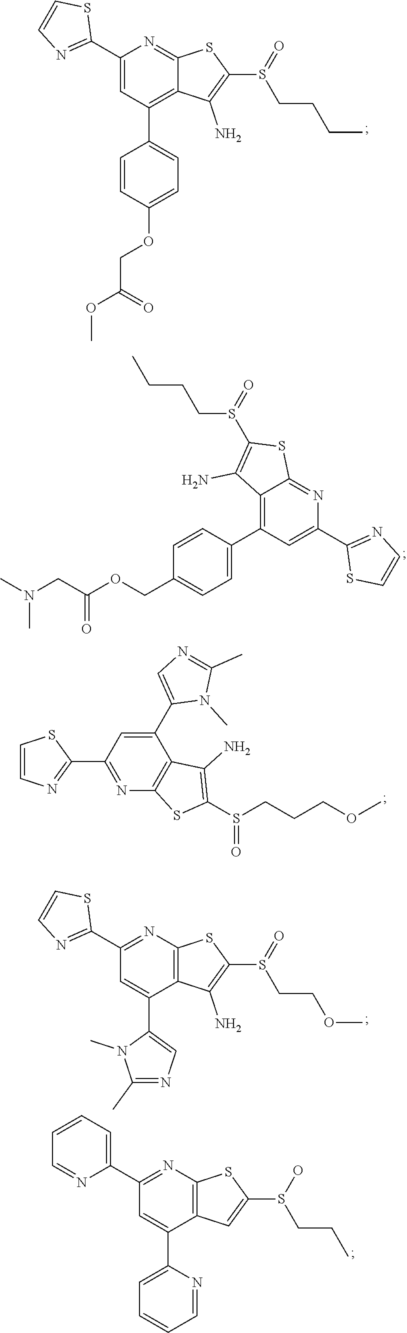

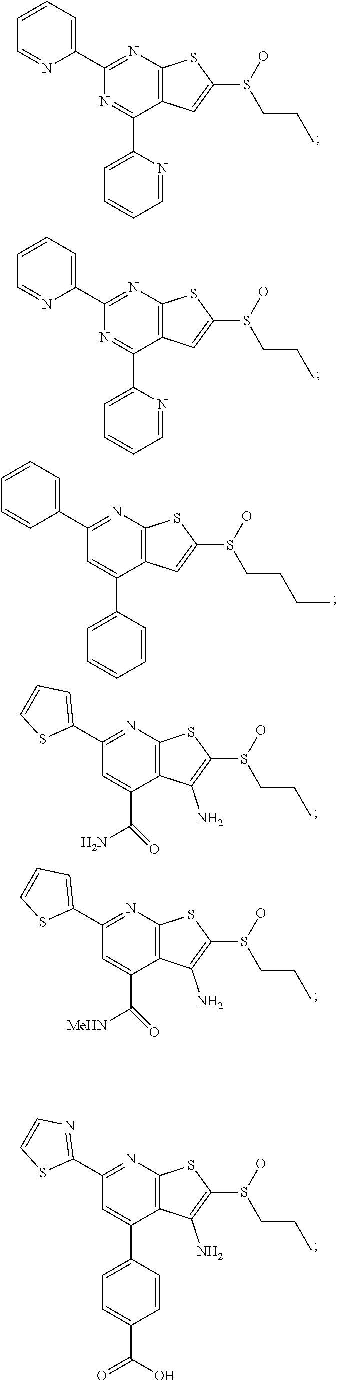

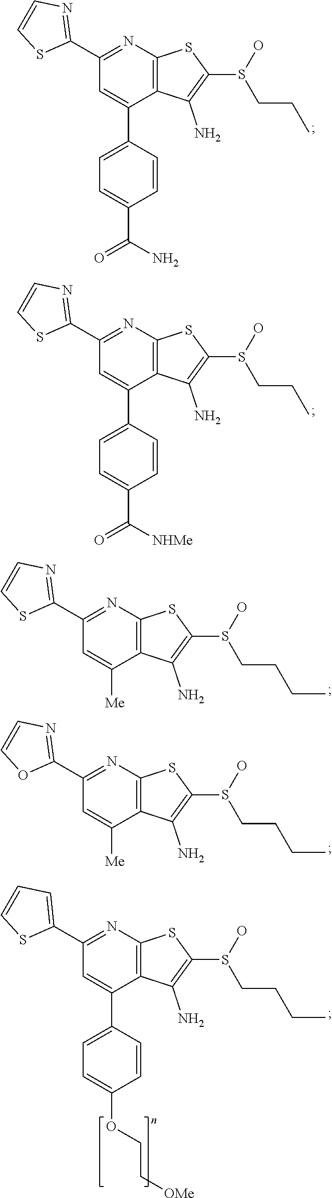

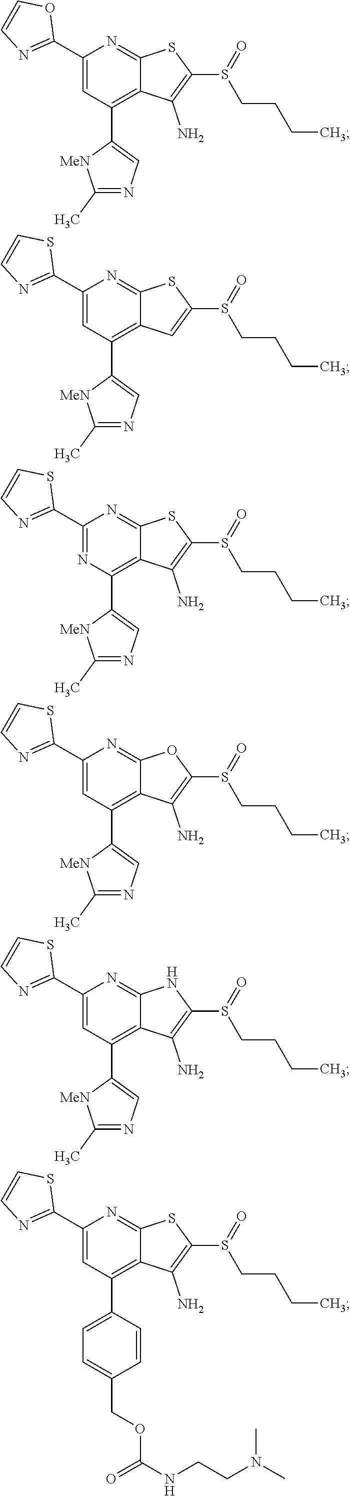

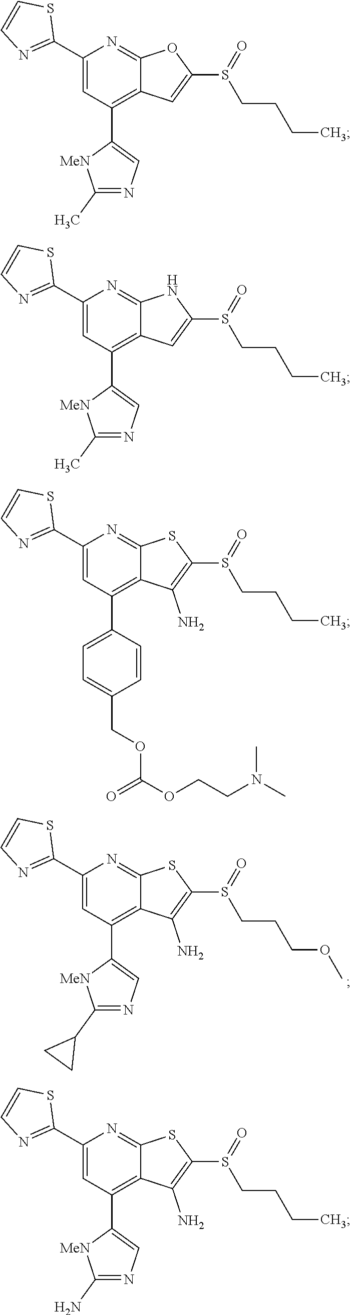

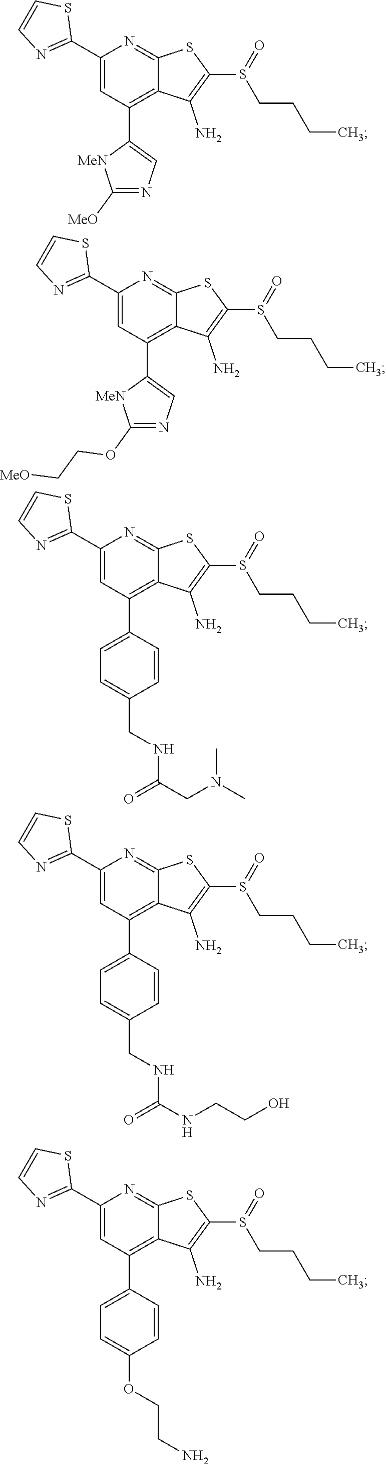

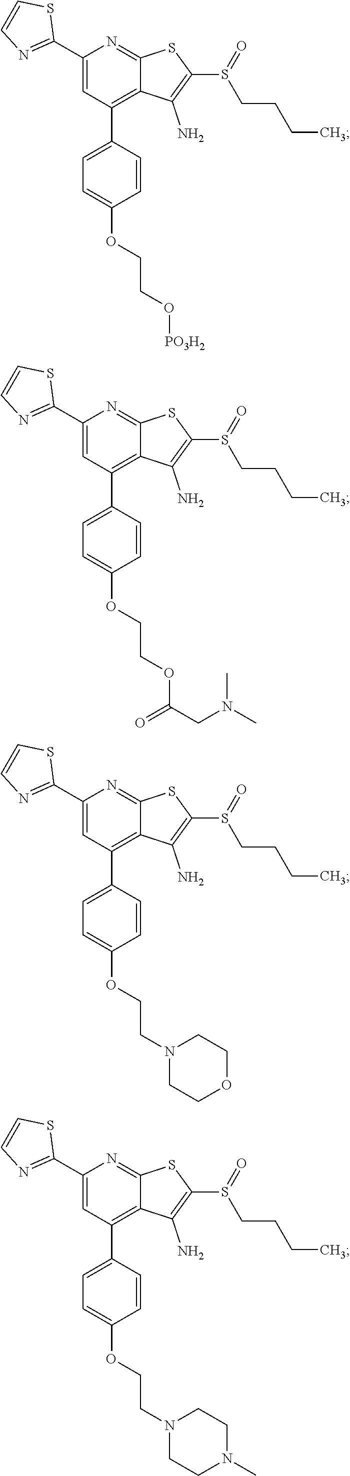

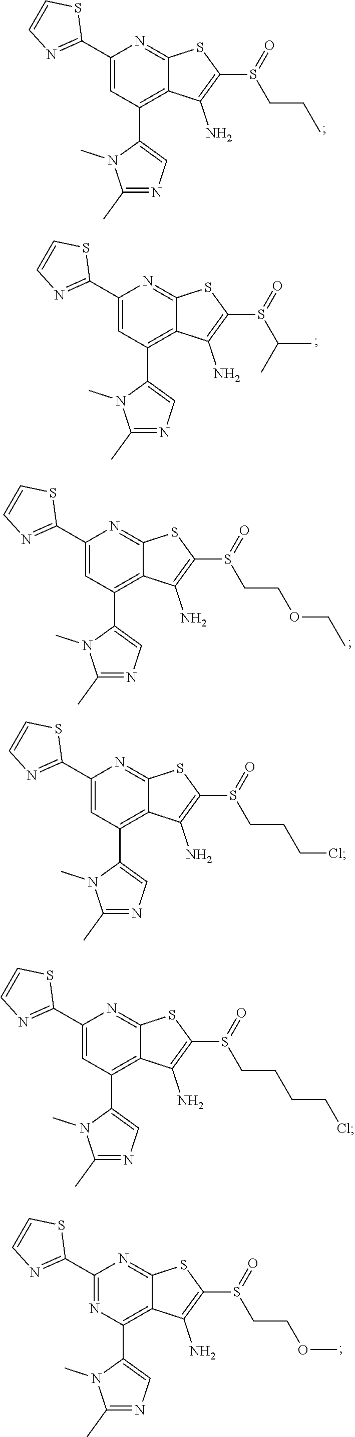

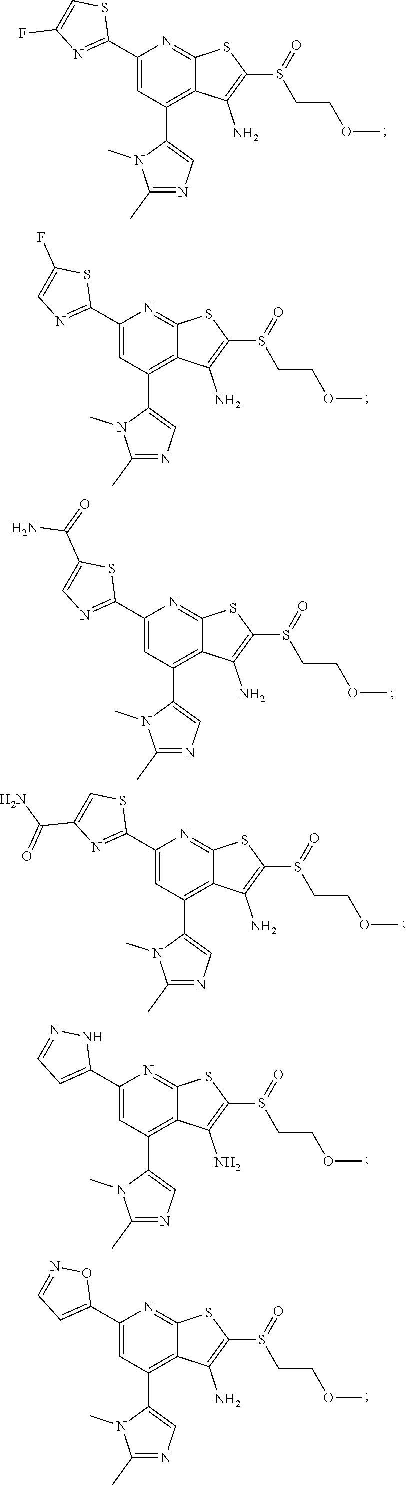

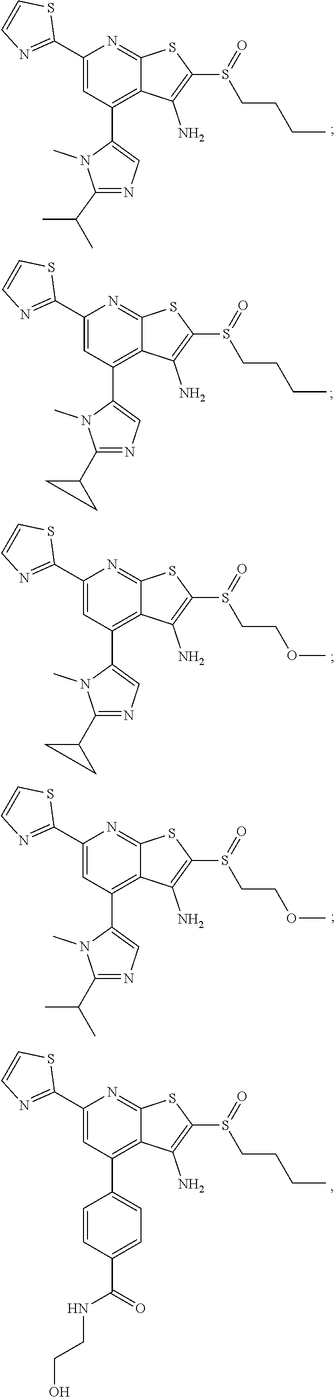

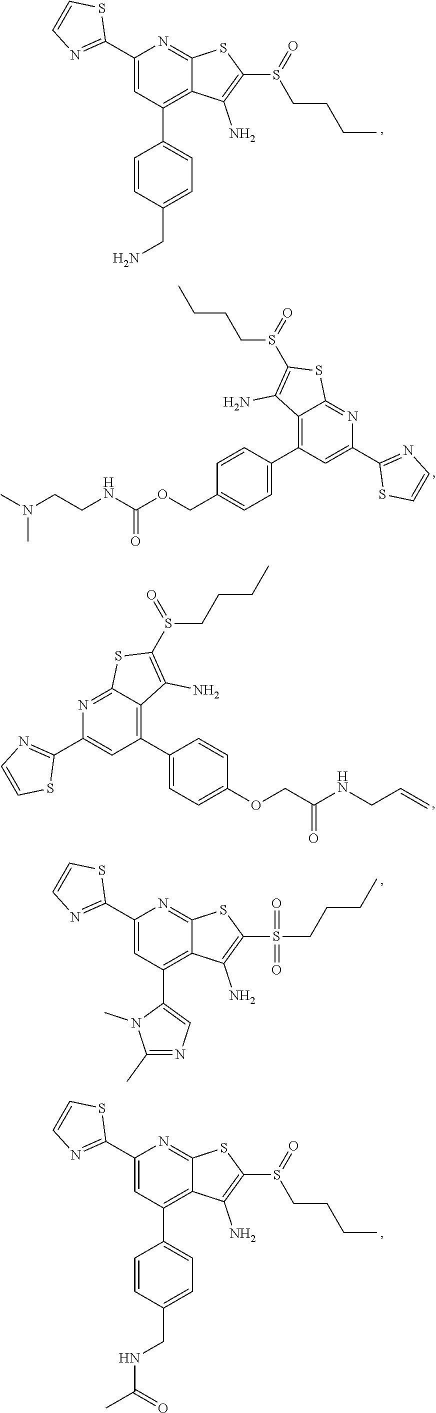

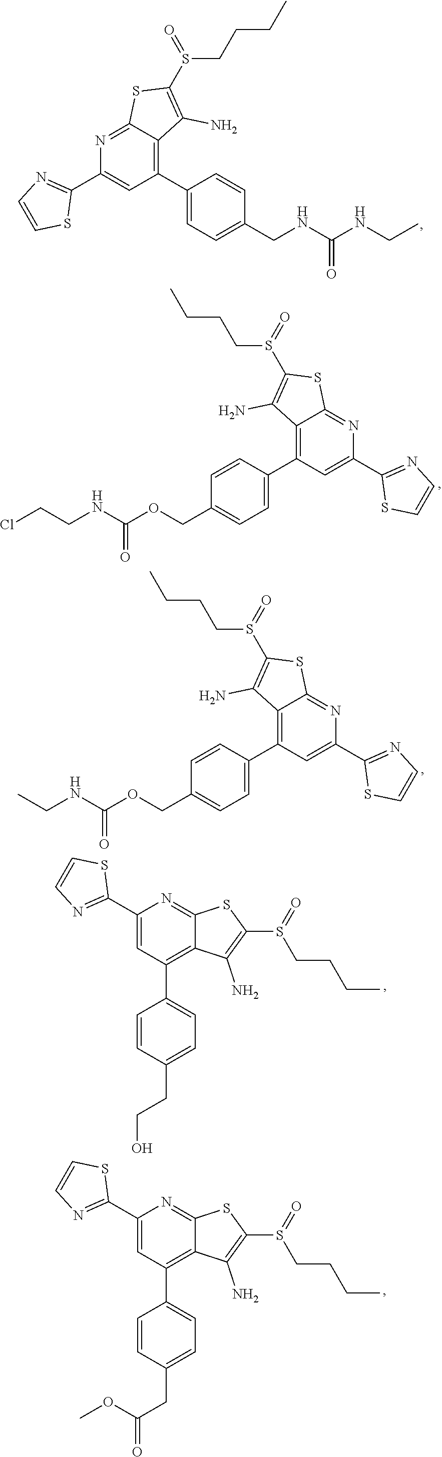

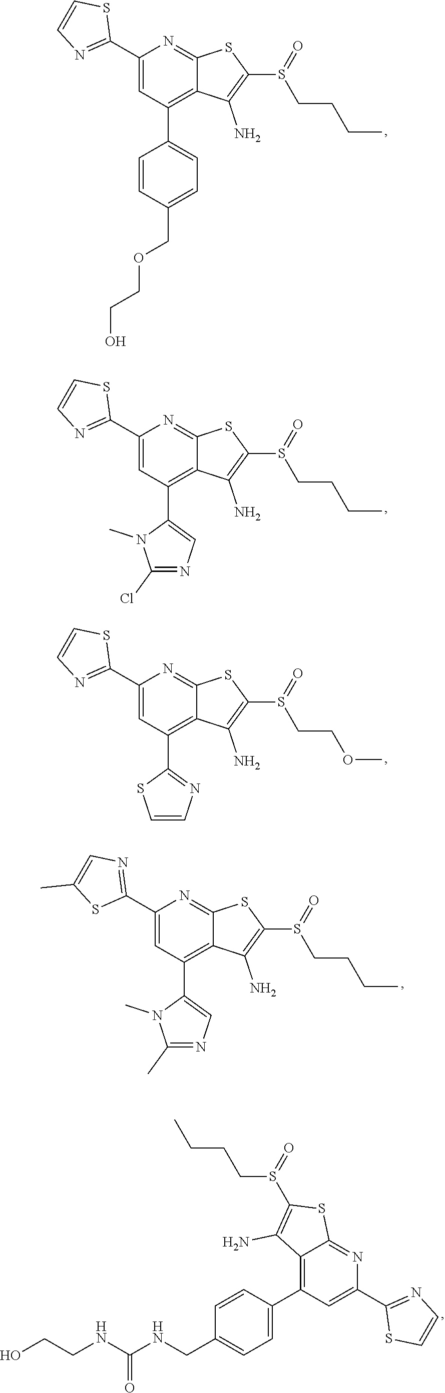

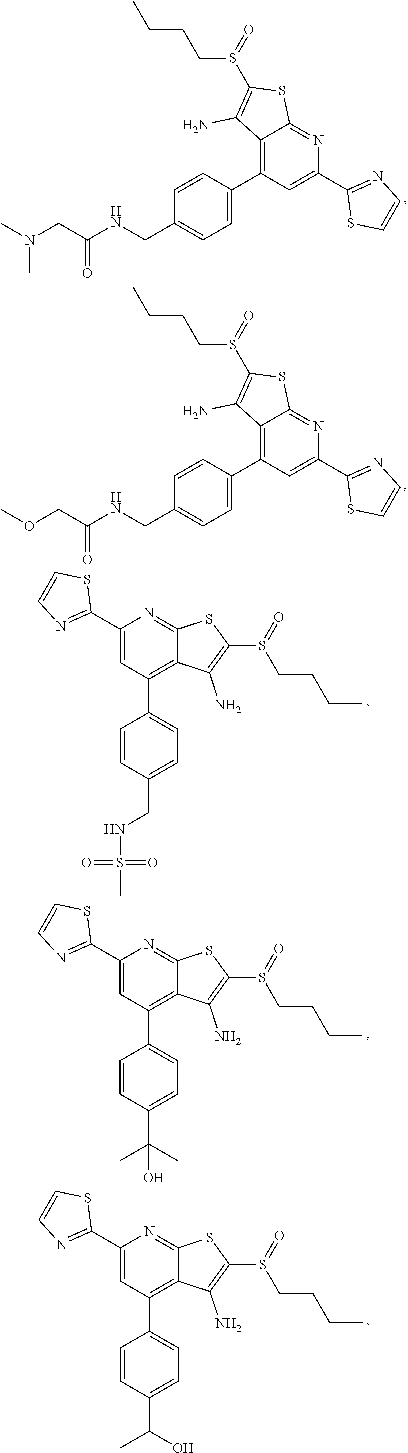

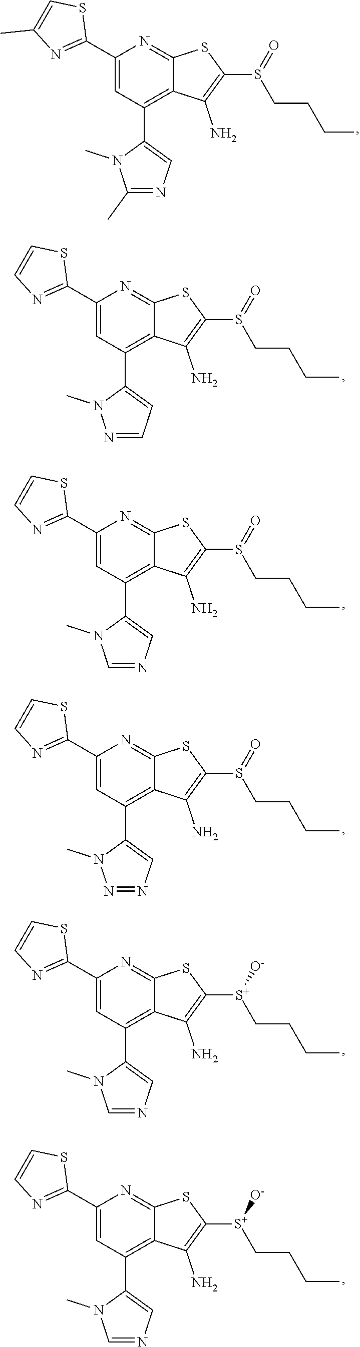

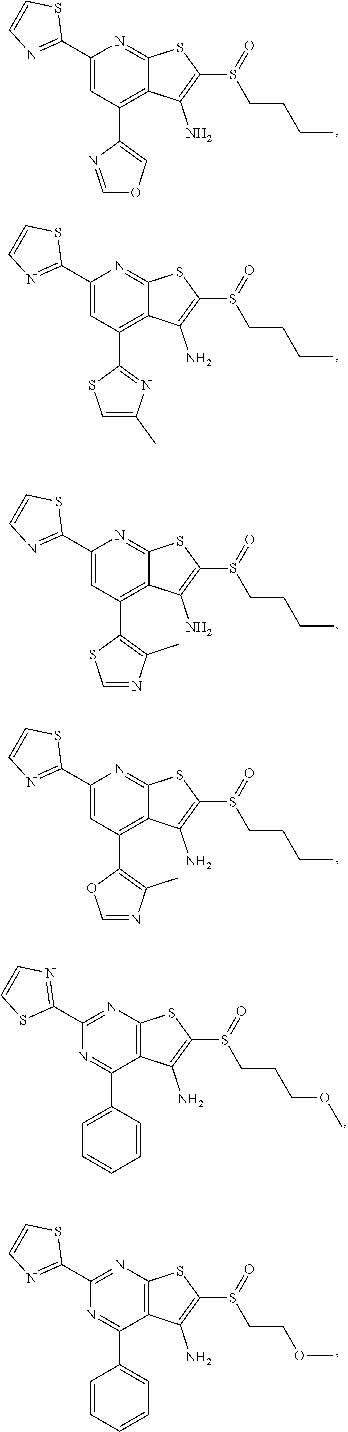 [SMILES: CCCCS(=O)c1sc2nc(-c3nccs3)cc(-c3cocn3)c2c1N ; CCCCS(=O)c1sc2nc(-c3nccs3)cc(-c3nc(C)cs3)c2c1N ; CCCCS(=O)c1sc2nc(-c3nccs3)cc(-c3scnc3C)c2c1N ; CCCCS(=O)c1sc2nc(-c3nccs3)cc(-c3ocnc3C)c2c1N ; COCCCS(=O)c1sc2nc(-c3nccs3)nc(-c3ccccc3)c2c1N ; COCCS(=O)c1sc2nc(-c3nccs3)nc(-c3ccccc3)c2c1N]

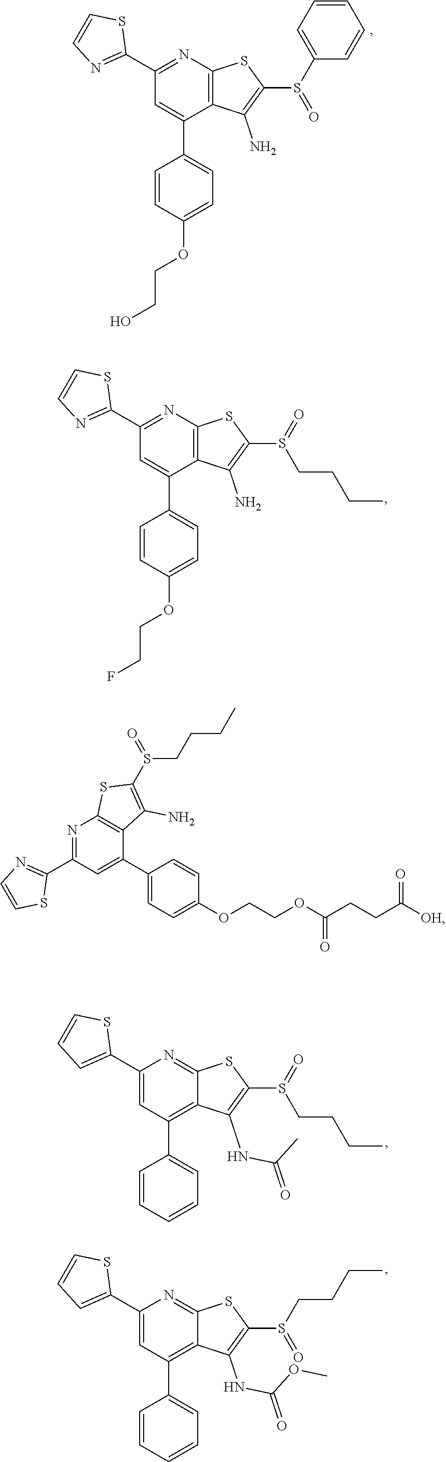

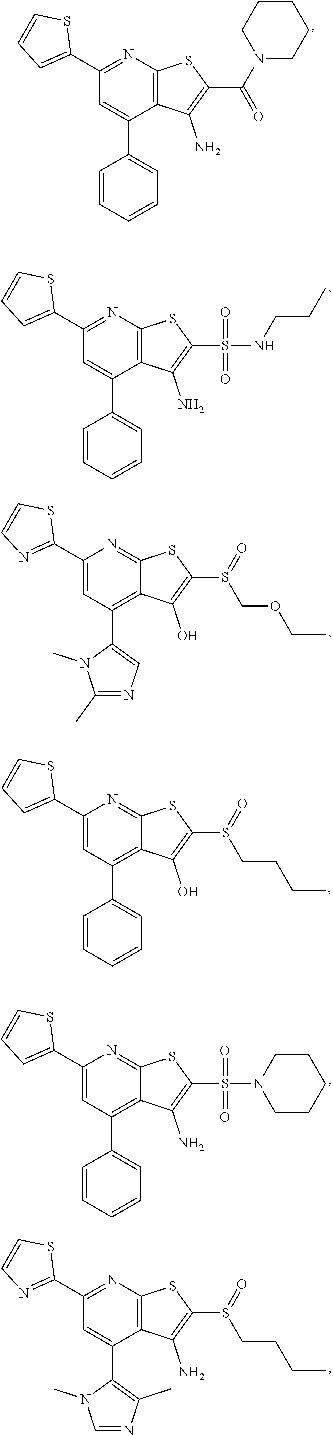 [SMILES: Nc1c(C(=O)N2CCCCC2)sc2nc(-c3cccs3)cc(-c3ccccc3)c12 ; CCCNS(=O)(=O)c1sc2nc(-c3cccs3)cc(-c3ccccc3)c2c1N ; CCOCS(=O)c1sc2nc(-c3nccs3)cc(-c3cnc(C)n3C)c2c1O ; CCCCS(=O)c1sc2nc(-c3cccs3)cc(-c3ccccc3)c2c1O ; Nc1c(S(=O)(=O)N2CCCCC2)sc2nc(-c3cccs3)cc(-c3ccccc3)c12 ; CCCCS(=O)c1sc2nc(-c3nccs3)cc(-c3c(C)ncn3C)c2c1N]

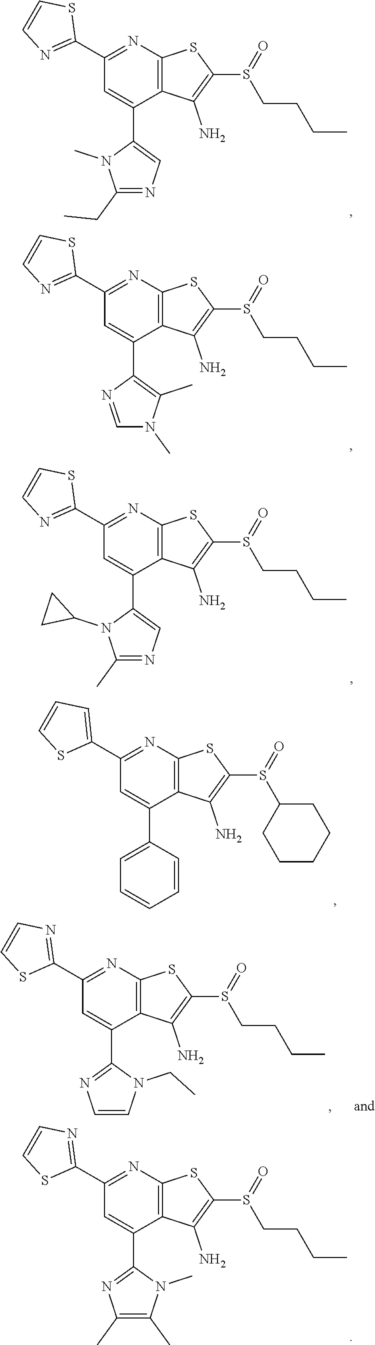

The inventors performed various assays to investigate the activity of 15-PGDH inhibitors, and discovered that the substituent at the R⁴ position on Formula (I) was cleavagd during metabolism. Thus, the substituent at the R⁴ position can result in the presence of toxic metabolites. The inventors then discovered that 6-membered heteroaryl rings at the R⁴ position on Formula (I) have low risk of producing toxic metabolites. Non-limiting examples of such 6-membered heteroaryl rings include those having from 1 to 3 nitrogen atms (e.g., 1 or 2 nitrogen atoms), such as pyridinyl and pyrimidinyl rings.

In still other embodiments, the 15-PGDH inhibitor can include a compound having formula (II):

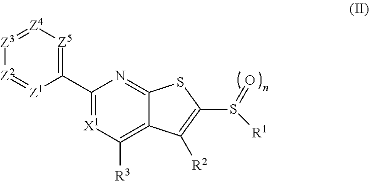

wherein n=0-2;
X¹ is N or CR^{a};
Z¹, Z², Z³, Z⁴, and Z⁵ are each independently N or CR^{b}, provided no more than 3 of Z¹, Z², Z³, Z⁴, and Z⁵ are N;
R¹ is selected from the group consisting of branched or linear alkyl including —(CH₂)n₁CH₃ (n₁=0-7),

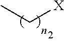

wherein n₂=0-6 and X is any of the following: CF_{y}H_{z} (y+z=3), CCl_{y}H_{z} (y+z=3), OH, OAc, OMe, R⁷¹, OR⁷², CN, N(R⁷³)₂,

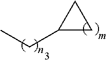

(n₃=0-5, m=1-5), and 4 (n₄=0-5).

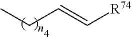

R² is selected from the group consisting of H, OH, linear or branched alkyl, Cl, F, NH₂, N(R⁷⁶)₂, and OR⁷⁷;
R³ is selected from the group consisting of:

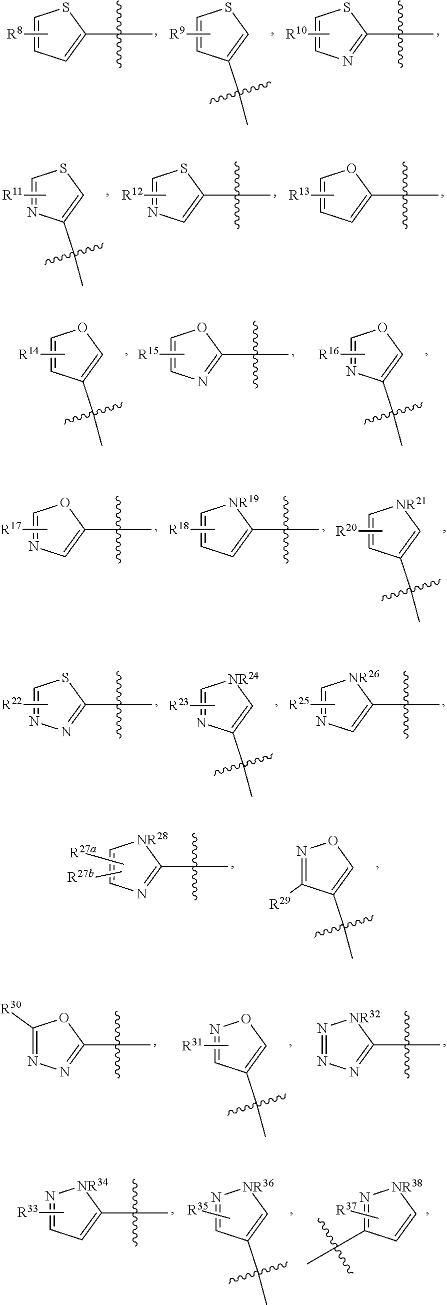

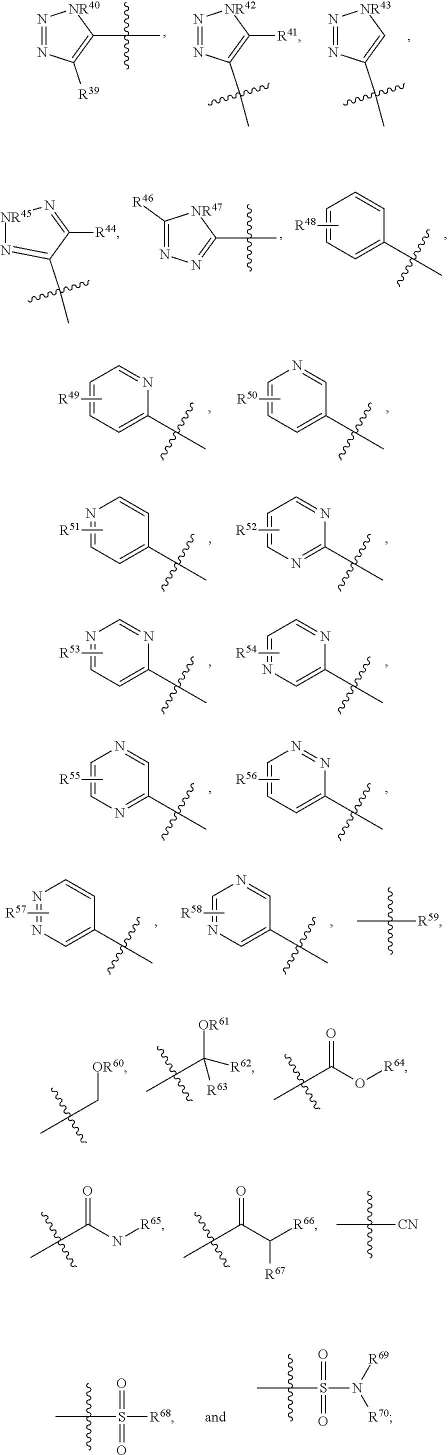

each R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁶, R⁷⁷, R^{a}, and R^{b} are the same or different and are independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 4-6 ring atoms, (wherein from 1-3 of the ring atoms is independently selected from N, NH, N(C₁-C₆ alkyl), NC(O)(C₁-C₆ alkyl), O, and S), heteroaryl or heterocyclyl containing from 4-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano(—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R═hydrogen, alkyl, aryl, alkaryl, aralkyl, etc.), arylimino (—CR═N(aryl), where R═hydrogen, alkyl, aryl, alkaryl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, arlyl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R═H, methyl or other alkyl], groups incorporating amino acids or other moieties expected to bear positive or negative charge at physiological pH, and combinations thereof; and
   pharmaceutically acceptable salts thereof; and wherein R³ is not

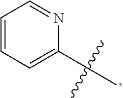 [SMILES: *c1ccccn1]

if one of Z¹ or Z⁵ is N and Z², Z³, and Z⁴ are CH.

In some embodiments, R¹ is a —(CH₂)n₁CH₃ (n₁=1, 2, 3, 4, or 5) or

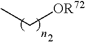

wherein n₂=1, 2, 3, 4, or 5.

In other embodiments, R² is —NH₂.

In other embodiments, R³ is

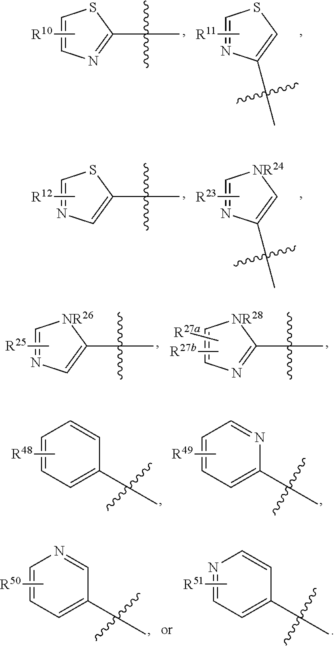

In some embodiments, each R¹⁰, R¹¹, R¹², R²³, R²⁴, R²⁵, R²⁶, R^{27a}, R^{27b}, R²⁸, R⁴⁸, R⁴⁹, R⁵⁰ and R⁵¹ are the same or different independently selected from hydrogen, halo, —C₁-C₆ alkyl, —C₃-C₆ cycloalkyl, and cyano.

In other embodiments, each R^{b} is the same or different and independently selected from hydrogen, halo, —NH₂, —NHC₁-C₃ alkyl, —N(C₁-C₃ alkyl)₂, —O—C₁-C₃ alkyl, and heterocyclyl containing from 4-6 ring atoms (wherein 1 atom of the ring atoms is independently selected from O), and pharmaceutically acceptable salts thereof.

In some embodiments, n=1.

In other embodiments, a compound having a structure according formula (II) can have a structure according to formula (IIA) to (IIH):

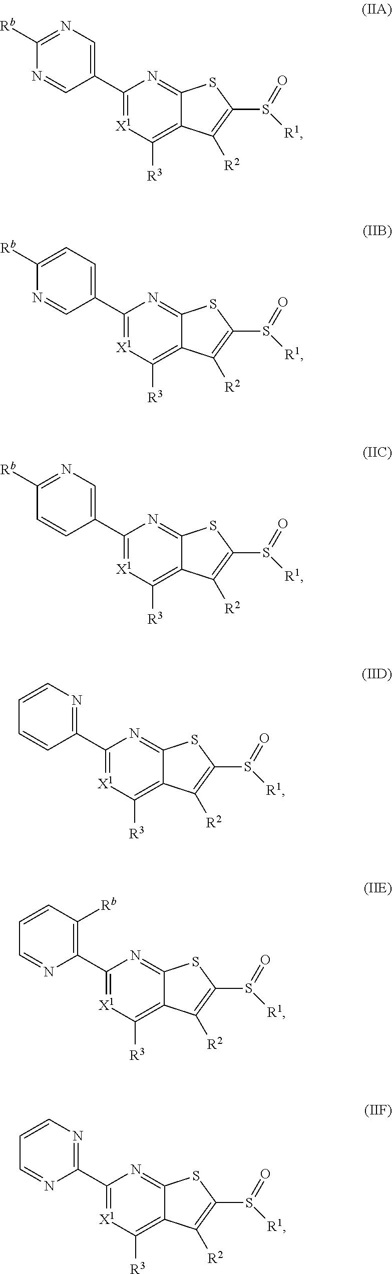

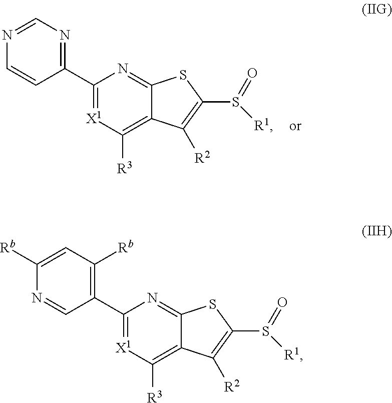

wherein:
R¹ is a —(CH₂)n₁CH₃ (n₁=1, 2, 3, 4, or 5), or

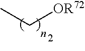

wherein n₂=1, 2, 3, 4, or 5;
R² is —NH₂
R³ is

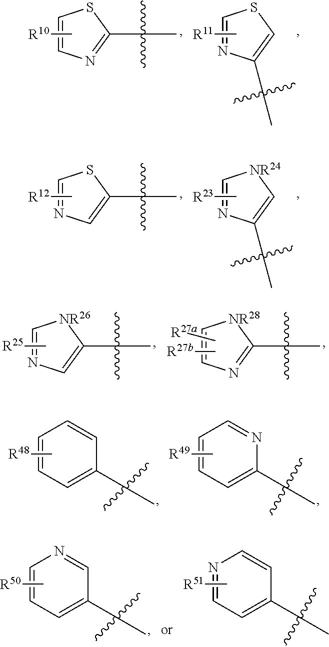

each of R¹⁰, R¹¹, R¹², R²³, R²⁴, R²⁵, R²⁶, R^{27a}, R^{27b}, R²⁸, R⁴⁸, R⁴⁹, and R⁵⁰ is the same or different and independently selected from hydrogen, halo, —C₁-C₃ alkyl, —C₃-C₆ cycloalkyl, or cyano;
each R^{b} is the same or different and independently selected from hydroge, halo, —NH₂, —NHC₁-C₃ alkyl, —N(C₁-C₃ alkyl)₂, —O—C₁-C₃ alkyl, and heterocyclyl containing from 4-6 ring atoms (wherein 1 atom of the ring atoms is independently selected from O), and pharmaceutically acceptable salts thereof.

In other embodiments, a compound having a structure according formula (II) can have a structure according to one of formula (IIA) to (IIH):

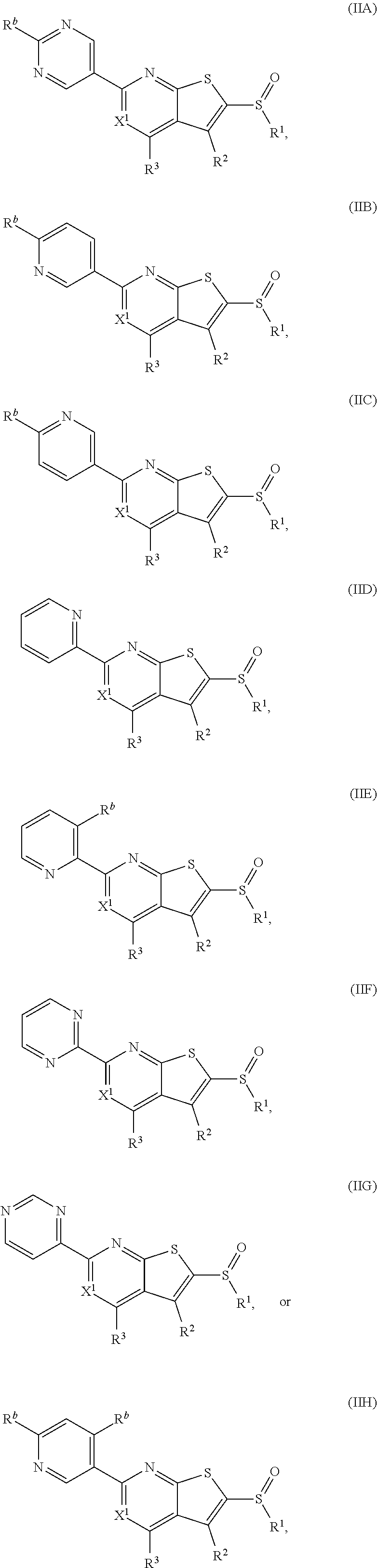

wherein:
R¹ is a —(CH₂)n₁CH₃ (n₁=1, 2, 3, 4, or 5), or

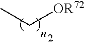

wherein n₂=1, 2, 3, 4, or 5;
R² is —NH₂′
R³ is

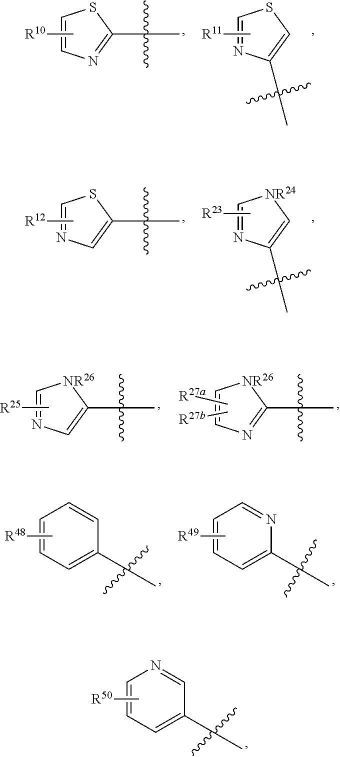

each R¹⁰, R¹¹, R¹², R²³, R²⁴, R²⁵, R²⁶, R^{27a}, R^{27b}, R²⁸, R⁴⁸, R⁴⁹, and R⁵⁰ is the same or different and independently selected from hydrogen, halo, —C₁-C₃ alkyl, —C₃-C₆ cycloalkyl, or cyano;
each R^{b} is the same or different and independently selected from hydrogen, halo, —NH₂, —NHC₁-C₆ alkyl, —N(C₁-C₆ alkyl)₂, —O—C₁-C₃ alkyl, heterocyclyl containing from 4-6 ring atoms, (wherein 1 atom of the ring atoms is O);
and pharmaceutically acceptable salts thereof.

Examples of compounds having formula (II) are selected from the group consisting of:

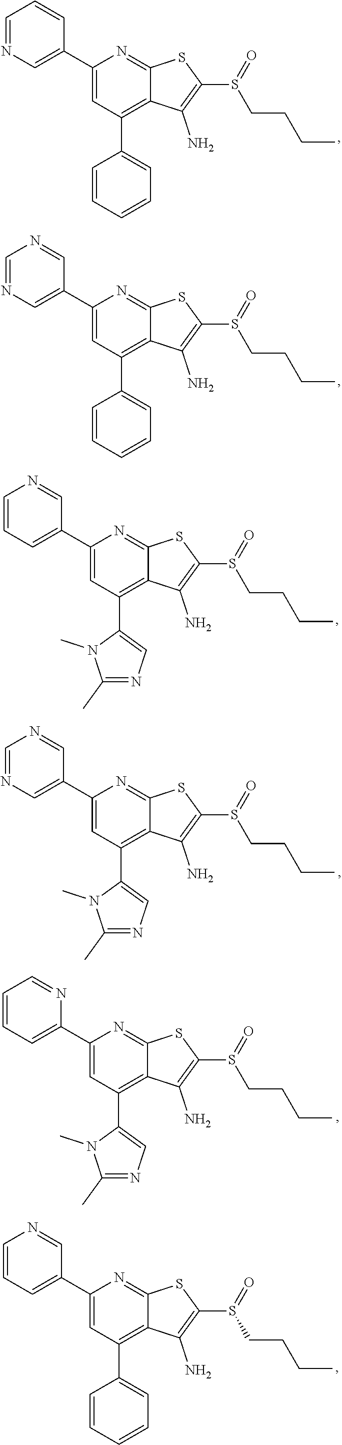

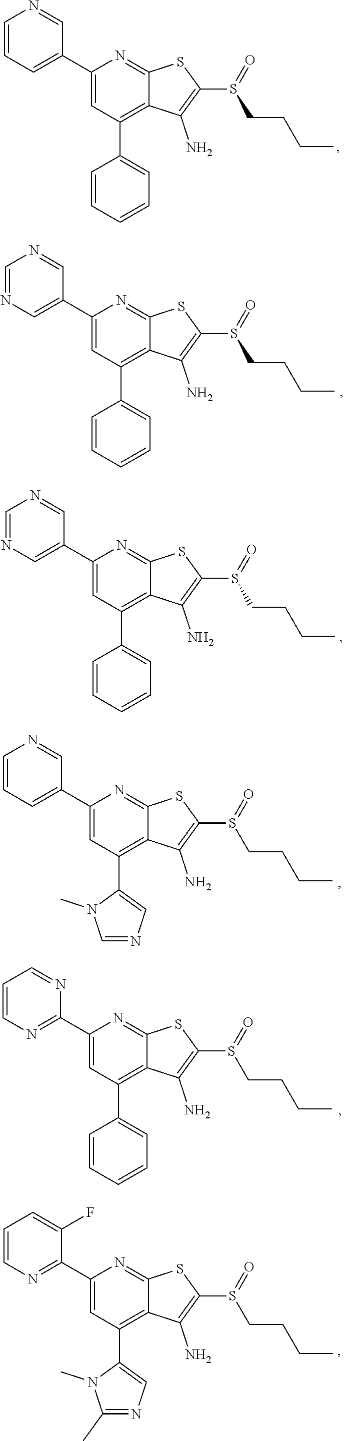

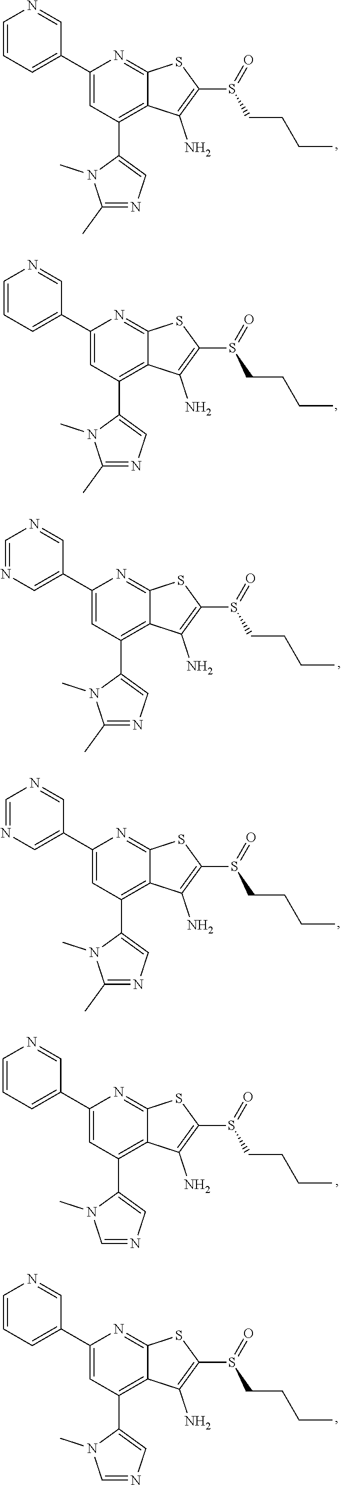 [SMILES: CCCC[S@@](=O)c1sc2nc(-c3cccnc3)cc(-c3cnc(C)n3C)c2c1N ; CCCC[S@](=O)c1sc2nc(-c3cccnc3)cc(-c3cnc(C)n3C)c2c1N ; CCCC[S@@](=O)c1sc2nc(-c3cncnc3)cc(-c3cnc(C)n3C)c2c1N ; CCCC[S@](=O)c1sc2nc(-c3cncnc3)cc(-c3cnc(C)n3C)c2c1N ; CCCC[S@@](=O)c1sc2nc(-c3cccnc3)cc(-c3cncn3C)c2c1N ; CCCC[S@](=O)c1sc2nc(-c3cccnc3)cc(-c3cncn3C)c2c1N]

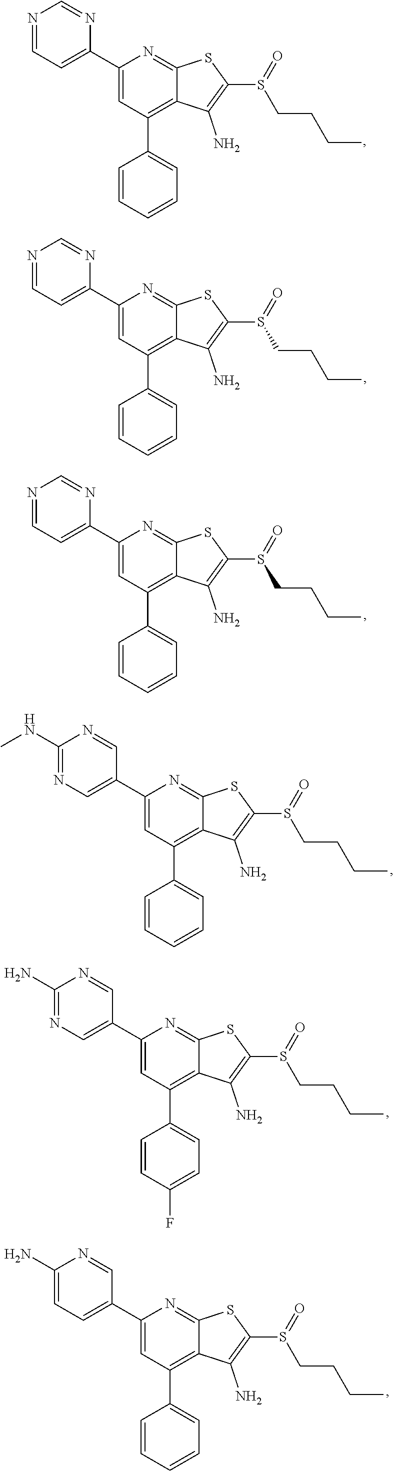

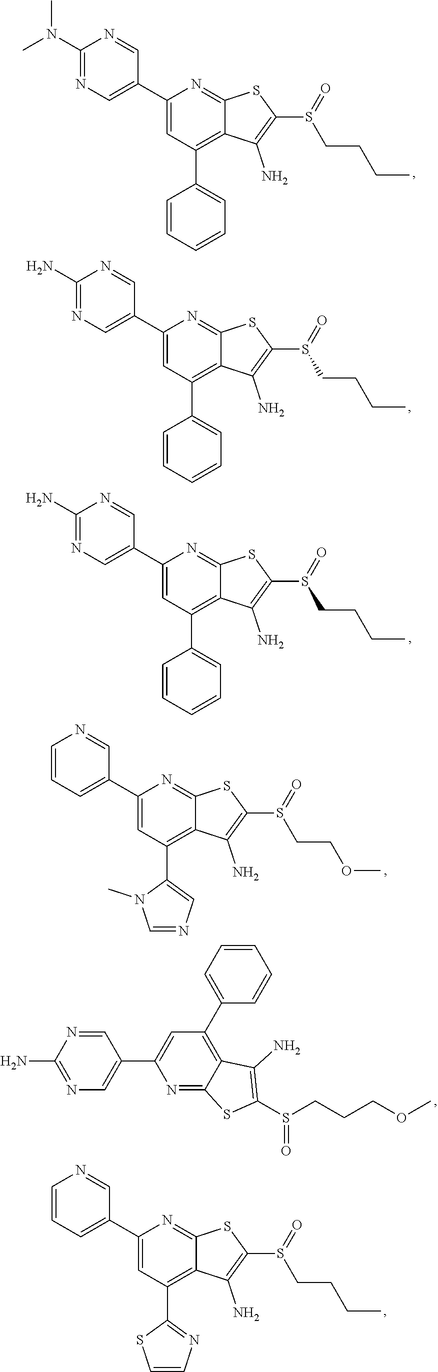

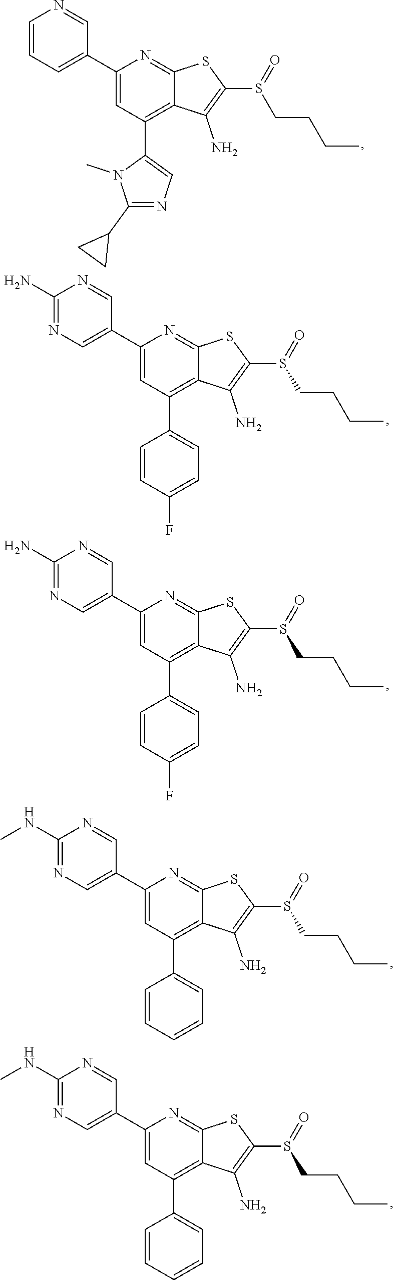

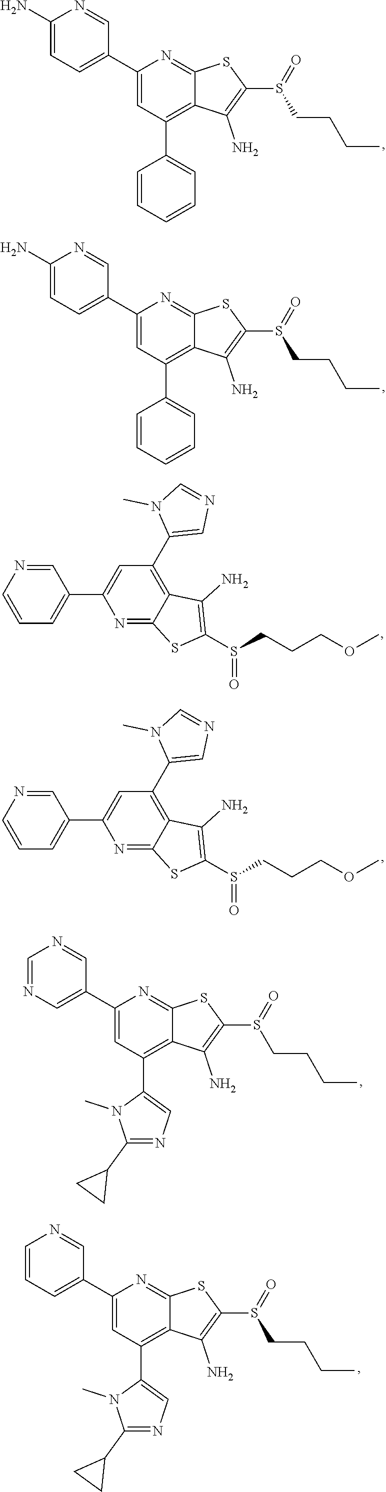

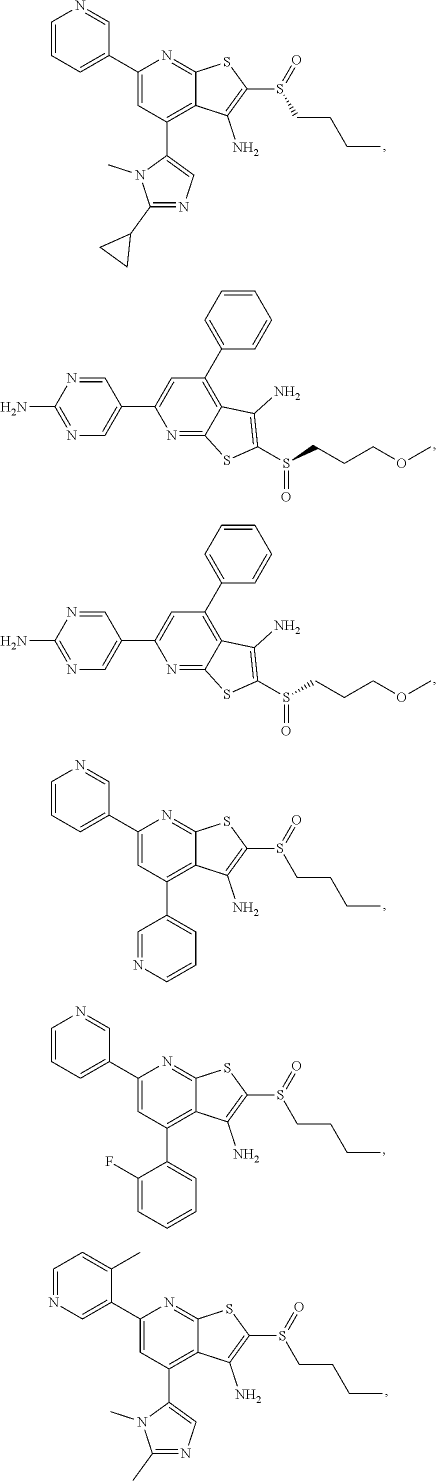

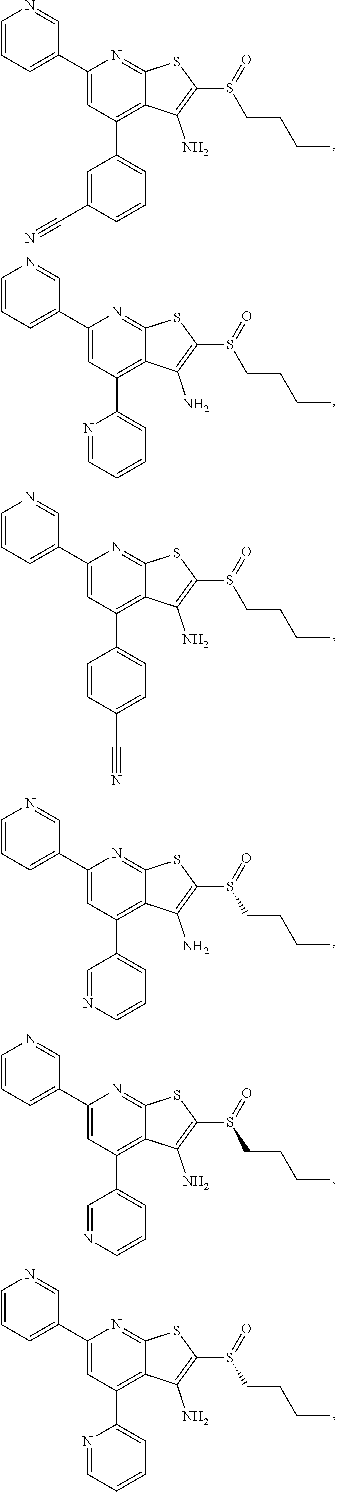

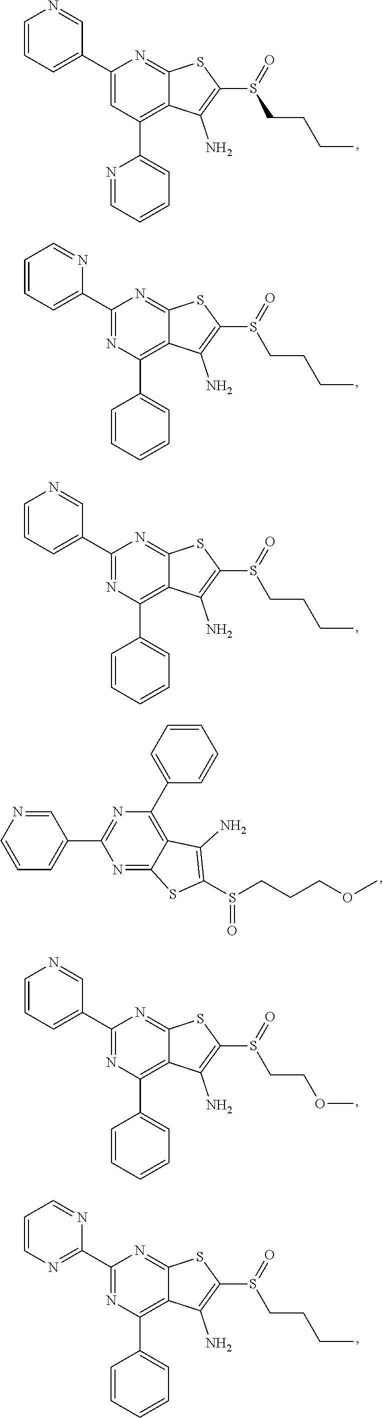

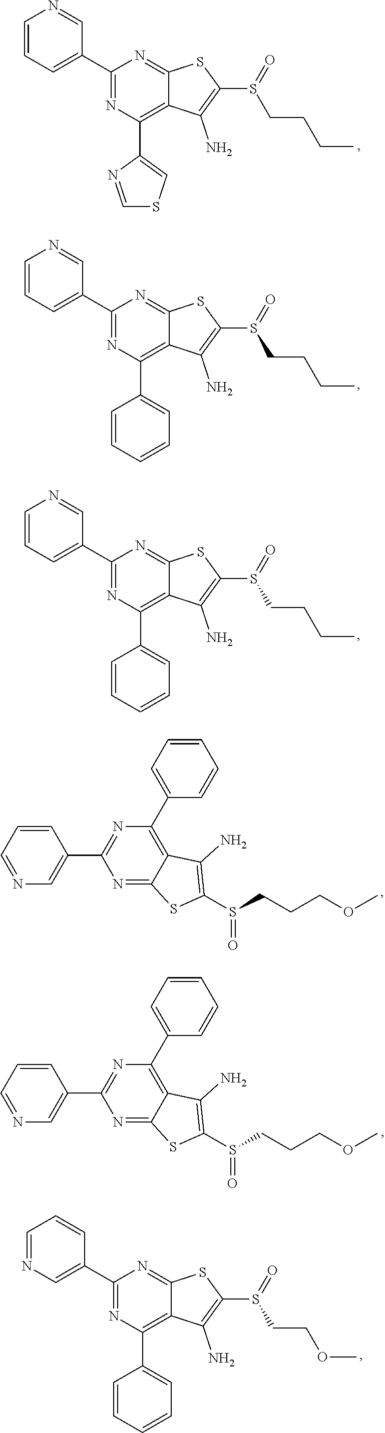

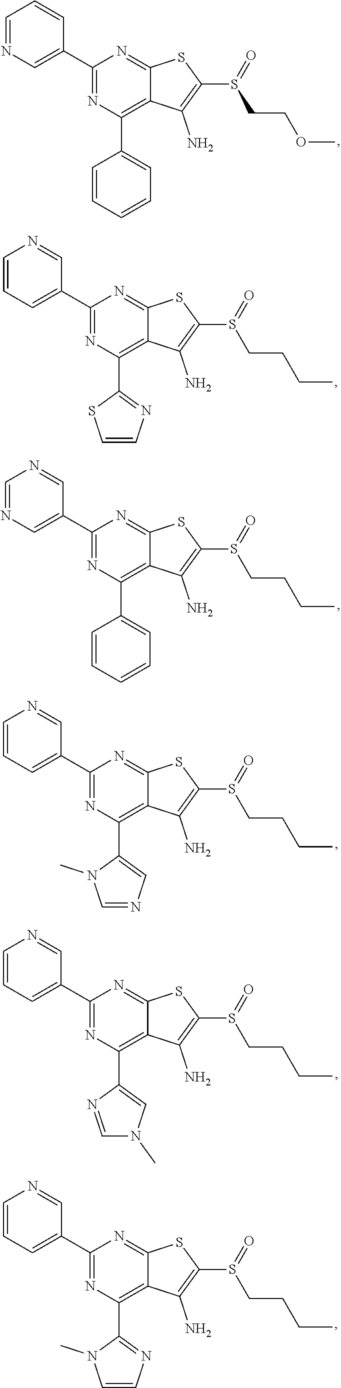 [SMILES: COCC[S@](=O)c1sc2nc(-c3cccnc3)nc(-c3ccccc3)c2c1N ; CCCCS(=O)c1sc2nc(-c3cccnc3)nc(-c3nccs3)c2c1N ; CCCCS(=O)c1sc2nc(-c3cncnc3)nc(-c3ccccc3)c2c1N ; CCCCS(=O)c1sc2nc(-c3cccnc3)nc(-c3cncn3C)c2c1N ; CCCCS(=O)c1sc2nc(-c3cccnc3)nc(-c3cn(C)cn3)c2c1N ; CCCCS(=O)c1sc2nc(-c3cccnc3)nc(-c3nccn3C)c2c1N]

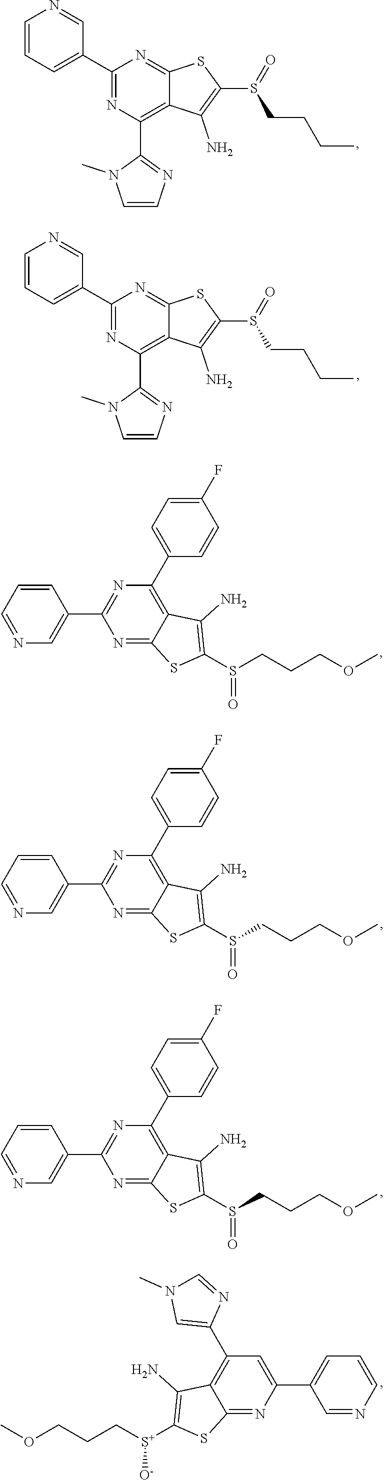

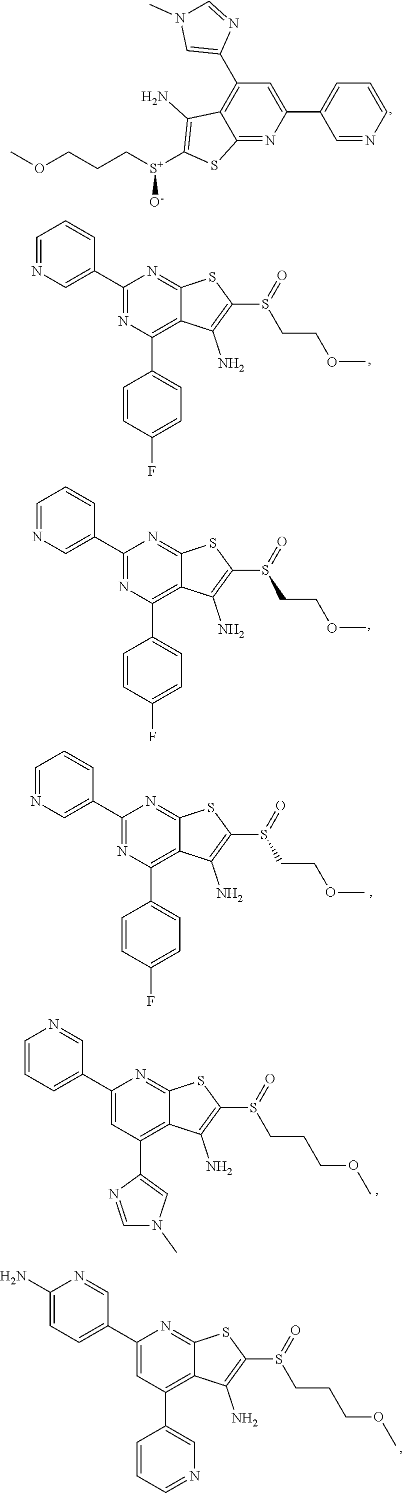 [SMILES: COCCC[S@+]([O-])c1sc2nc(-c3cccnc3)cc(-c3cn(C)cn3)c2c1N ; COCCS(=O)c1sc2nc(-c3cccnc3)nc(-c3ccc(F)cc3)c2c1N ; COCC[S@](=O)c1sc2nc(-c3cccnc3)nc(-c3ccc(F)cc3)c2c1N ; COCC[S@@](=O)c1sc2nc(-c3cccnc3)nc(-c3ccc(F)cc3)c2c1N ; COCCCS(=O)c1sc2nc(-c3cccnc3)cc(-c3cn(C)cn3)c2c1N ; COCCCS(=O)c1sc2nc(-c3ccc(N)nc3)cc(-c3cccnc3)c2c1N]

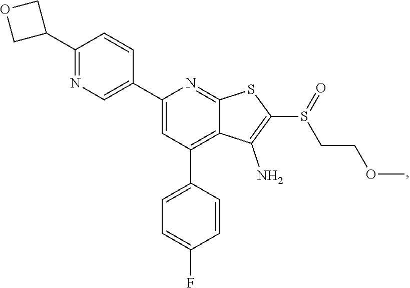 [SMILES: COCCS(=O)c1sc2nc(-c3ccc(C4COC4)nc3)cc(-c3ccc(F)cc3)c2c1N]

and pharmaceutically acceptable salts thereof.

In other embodiments, a compound having formula (II) does not have the following formula:

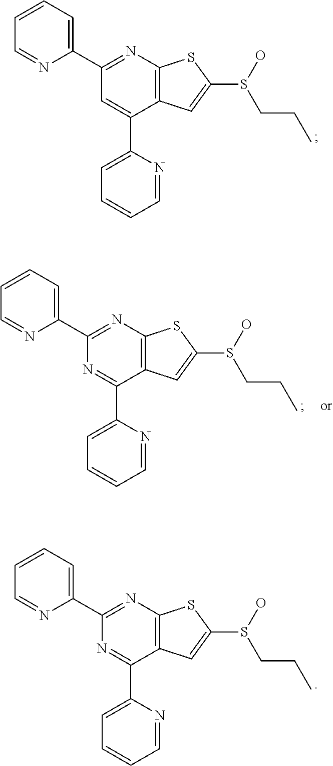

In some embodiments compounds of formulas of (I), which include 6-membered heteroaryl rings, such as pyridinyl and pyrimidinyl rings, at the R⁴ position on formula (I), compared to compounds of formula (I), which include a 5-membered heterocyle ring, such as a thiazole ring, at the R⁴ position can (i) induce an increase in the cellular levels of PGE-2 following IL1-beta stimulation of A459 cells at an EC50 less than about 50 nM, less than about 40 nm, less than about 30 nm, less than about 20 nm, or less than about 10 nm, and/or (ii) at a concentration of 10 μM have a metabolic stability as determined by half-life of the compounds incubated in the presence of microsomes derived from mouse livers of greater than about 5 min, greater than about 10 min, or greater than about 15 min.

From the various assays performed to investigate the activity of 15-PGDH inhibitors, the inventors also discovered that certain substituent at the R¹ position on formula (I) and (II) was readily metabolized, which limited exposure to the 15-PGDH inhibitors. The inventors then discovered that ethers at the R¹ position on formula (I) and (II) are less susceptible to metabolism, and therefore can improve to the 15-PGDH inhibitors. Non-limiting examples of ethers include those having a sulfur or oxygen heteroatom.

In other embodiments, the 15-PGDH inhibitor can include a compound having a structure according formula (III):

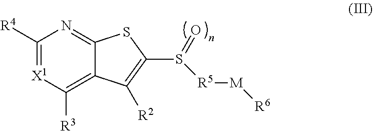

wherein n=0-2;
X¹ is N or CR^{a};
R² is selected from the group consisting of H, linear or branched alkyl, OH, Cl, F, NH₂, N(R⁷⁶)₂, and OR⁷⁷;
R³ and R⁴ are each independently selected from the group consisting of:

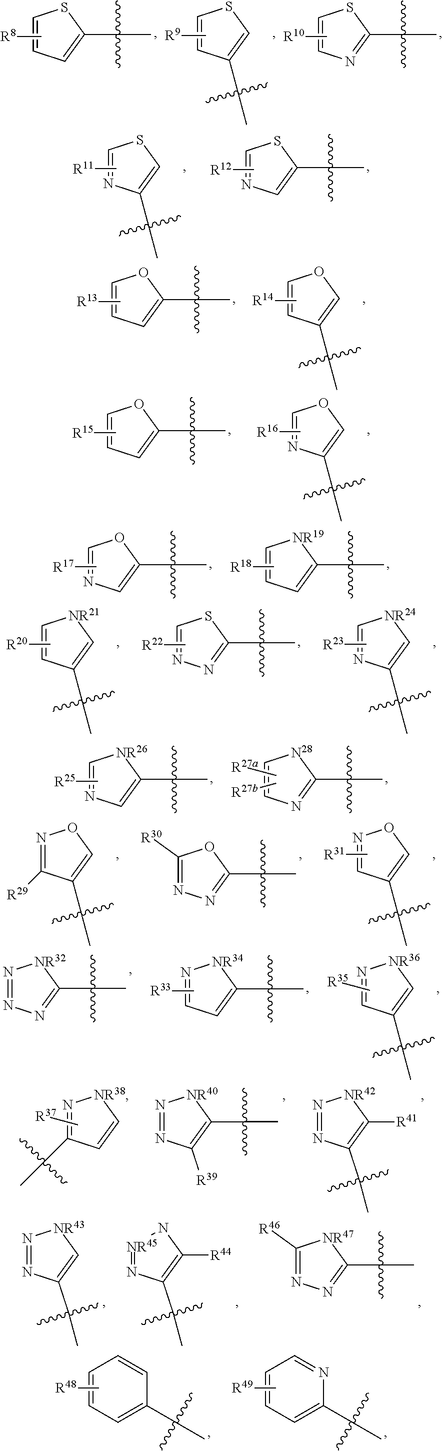

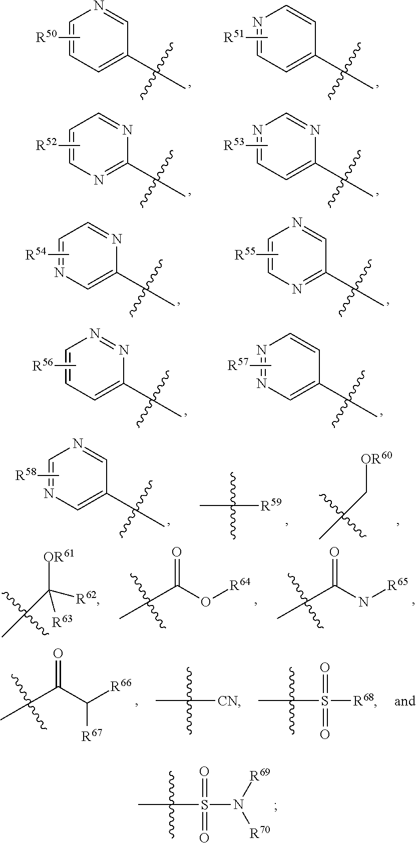

each R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷⁶, R⁷⁷, and R^{a} are the same or different and are independently selected from the group consisting of hydrogen, substituted or unsubstituted C₁-C₂₄ alkyl, C₂-C₂₄ alkenyl, C₂-C₂₄ alkynyl, C₆-C₂₀ aryl, heterocycloalkenyl containing from 4-6 ring atoms, (wherein from 1-3 of the ring atoms is independently selected from N, NH, N(C₁-C₆ alkyl), NC(O) (C₁-C₆ alkyl), O, and S), heteroaryl or heterocyclyl containing from 4-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano(—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R═hydrogen, alkyl, aryl, alkaryl, aralkyl, etc.), arylimino (—CR═N(aryl), where R═hydrogen, alkyl, aryl, alkaryl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, arlyl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R═H, methyl or other alkyl], groups incorporating amino acids or other moieties expected to bear positive or negative charge at physiological pH, and combinations thereof;
M is O or S;
R⁵ and R⁶ are each independently C₁-C₂₄ alkyl;
and pharmaceutically acceptable salts thereof.

In some embodiments, the compound of formula (III) is not a compound with a formula selected from the group consisting of:

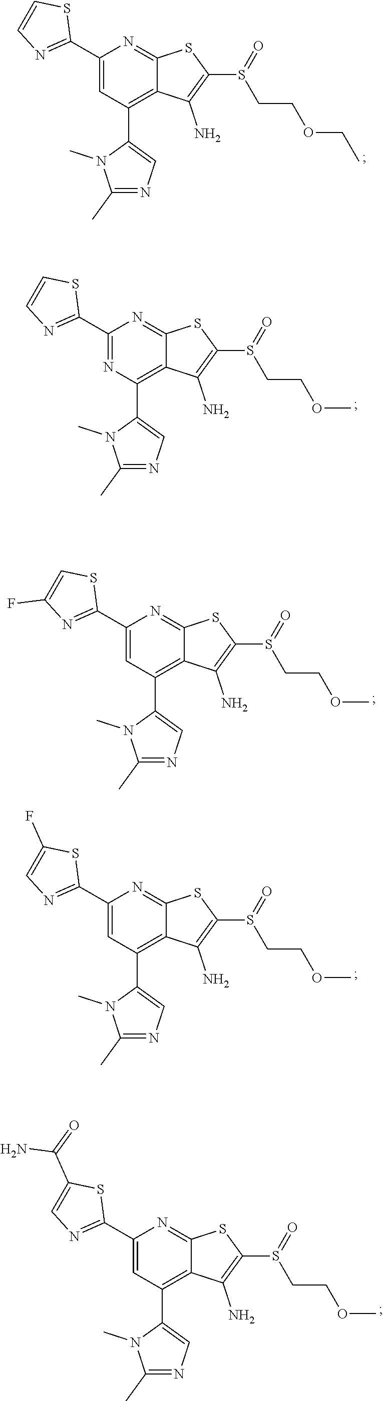 [SMILES: CCOCCS(=O)c1sc2nc(-c3nccs3)cc(-c3cnc(C)n3C)c2c1N ; COCCS(=O)c1sc2nc(-c3nccs3)nc(-c3cnc(C)n3C)c2c1N ; COCCS(=O)c1sc2nc(-c3nc(F)cs3)cc(-c3cnc(C)n3C)c2c1N ; COCCS(=O)c1sc2nc(-c3ncc(F)s3)cc(-c3cnc(C)n3C)c2c1N ; COCCS(=O)c1sc2nc(-c3ncc(C(N)=O)s3)cc(-c3cnc(C)n3C)c2c1N]

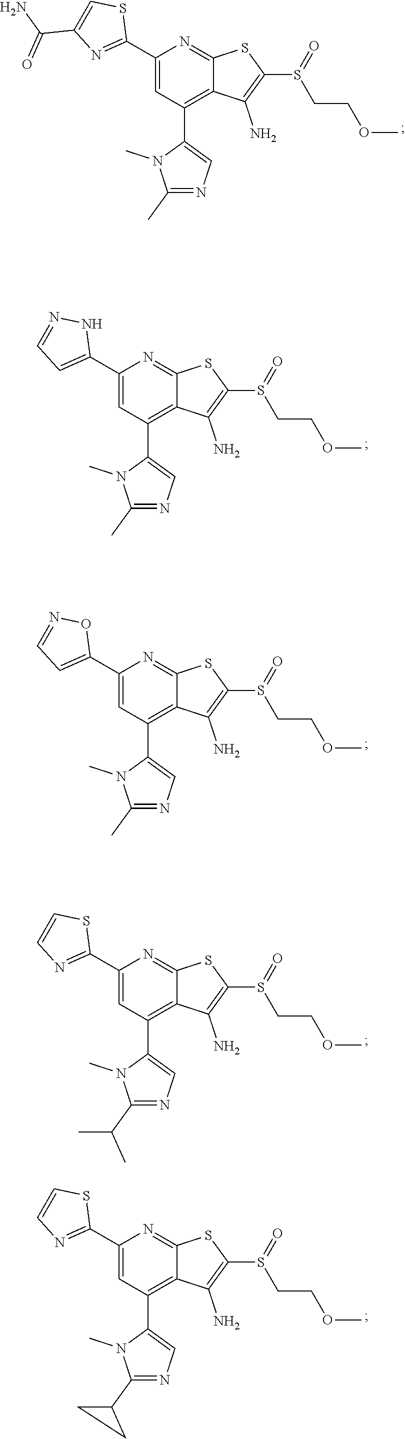 [SMILES: COCCS(=O)c1sc2nc(-c3nc(C(N)=O)cs3)cc(-c3cnc(C)n3C)c2c1N ; COCCS(=O)c1sc2nc(-c3ccn[nH]3)cc(-c3cnc(C)n3C)c2c1N ; COCCS(=O)c1sc2nc(-c3ccno3)cc(-c3cnc(C)n3C)c2c1N ; COCCS(=O)c1sc2nc(-c3nccs3)cc(-c3cnc(C(C)C)n3C)c2c1N ; COCCS(=O)c1sc2nc(-c3nccs3)cc(-c3cnc(C4CC4)n3C)c2c1N]

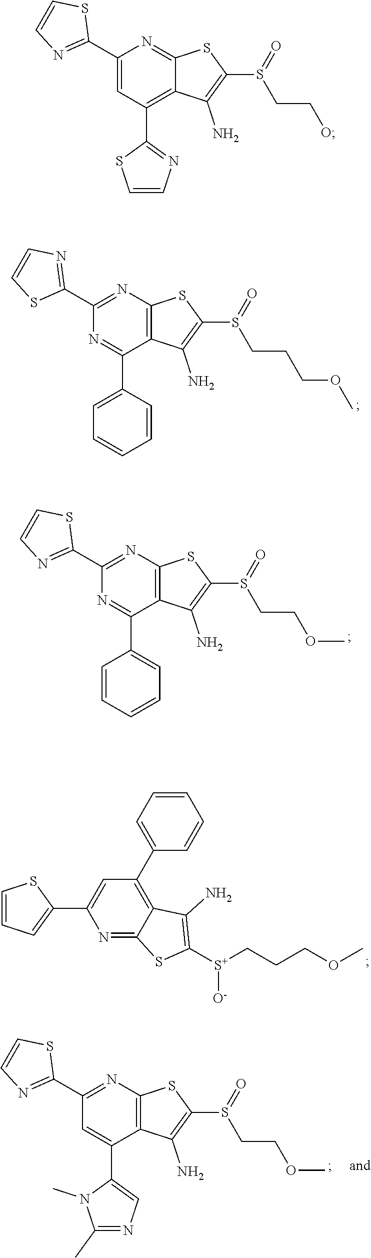

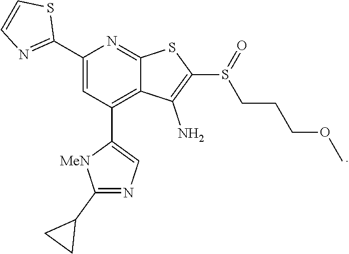 [SMILES: COCCCS(=O)c1sc2nc(-c3nccs3)cc(-c3cnc(C4CC4)n3C)c2c1N]

In some embodiments, R⁵ is a C₁-C₆ alkylene.

In other embodiments, R⁶ is a C₁-C₆ alkylene.

In still other embodiments, R⁵ is a C₂-C₅ alkylene and R⁶ is a C₁-C₃ alkylene.

In some embodiments, M is O.

In other embodiments, R² is NH₂.

In some embodiments, R³ and R⁴ are each independently

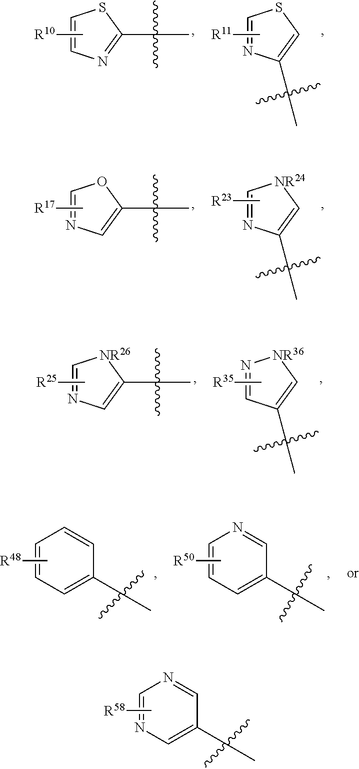

In some embodiments, each R¹⁰, R¹⁷, R²³, R²⁴, R²⁵, R²⁶, R³⁵, R³⁶, R⁴⁸, R⁵⁰, and R⁵¹ is the same or different and independently hydrogen, halo, —C₁-C₃ alkyl, —C₃-C₆ cycloalkyl, heterocyclyl containing from 4-6 ring atoms, (wherein 1 atom of the ring atoms is O), or cyano.

In other embodiments, n=1.

In still other embodiments, R^{a} is H.

In other embodiments, the 15-PGDH inhibitor can include a compound having a structure according formula (IIIA):

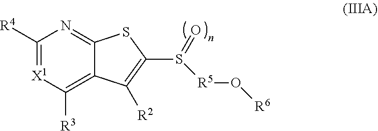

wherein n=1;
X¹ is N or CH;
R² is NH₂;
R³ and R⁴ are each independently

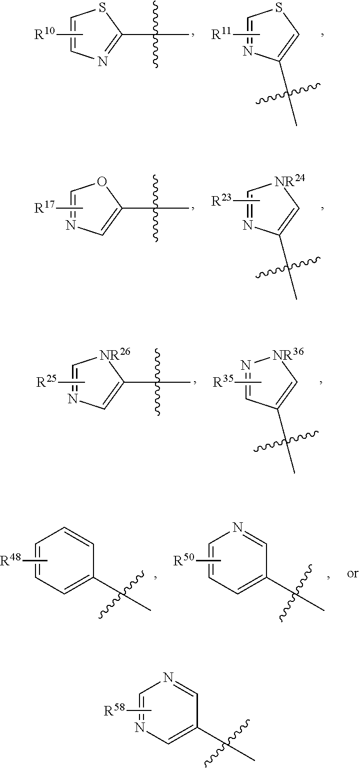

each R¹⁰, R¹⁷, R²³, R²⁴, R²⁵, R²⁶, R³⁵, R³⁶, R⁴⁸, R⁵⁰, and R⁵⁸ is the same or different and independently H, halo, —C₁-C₃ alkyl, —C₃-C₆ cycloalkyl, heterocyclyl containing from 4-6 ring atoms, (wherein 1 atom of the ring atoms is O), or cyano;
R⁵ and R⁶ are each independently C₁₋₆alkyl;
and pharmaceutically acceptable salts thereof.

Examples of compounds having formula (III) are selected from the group consisting of:

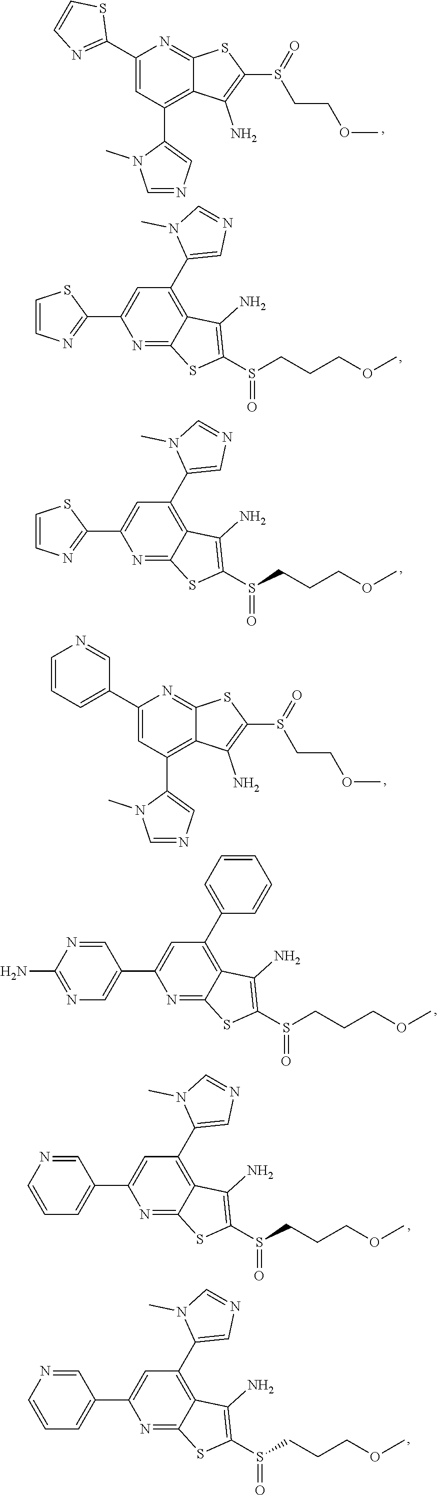

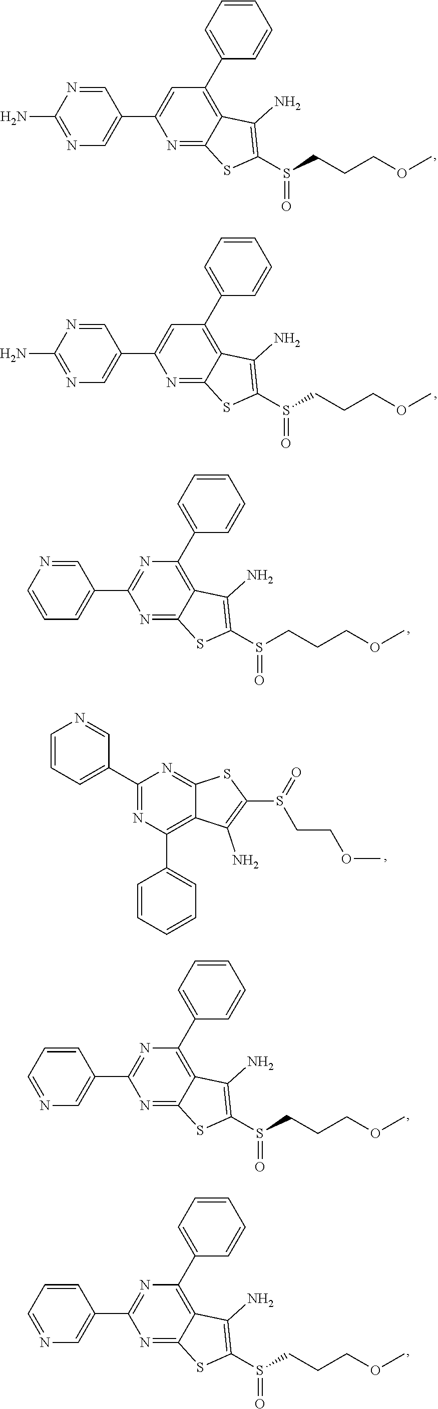

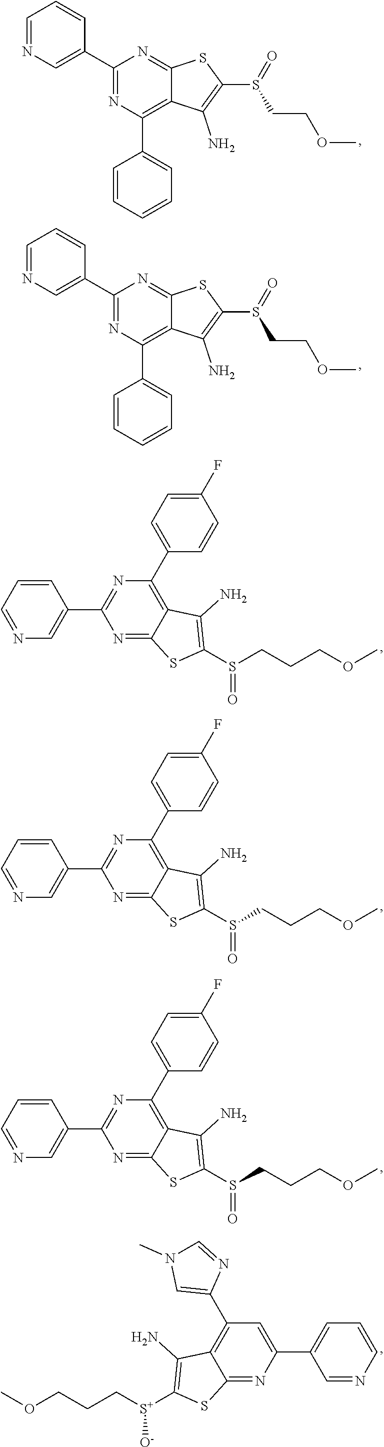

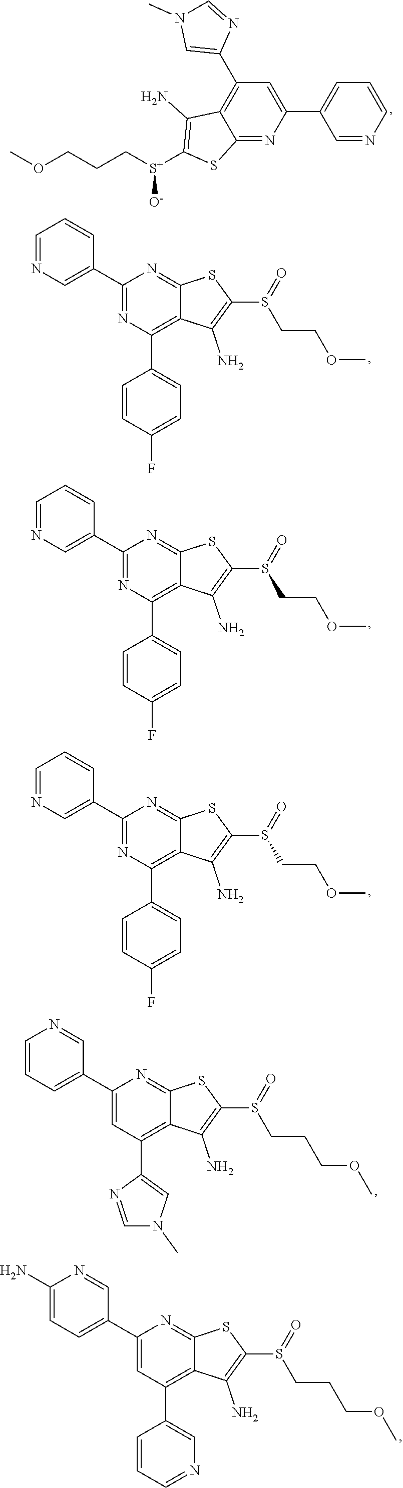 [SMILES: COCCC[S@+]([O-])c1sc2nc(-c3cccnc3)cc(-c3cn(C)cn3)c2c1N ; COCCS(=O)c1sc2nc(-c3cccnc3)nc(-c3ccc(F)cc3)c2c1N ; COCC[S@](=O)c1sc2nc(-c3cccnc3)nc(-c3ccc(F)cc3)c2c1N ; COCC[S@@](=O)c1sc2nc(-c3cccnc3)nc(-c3ccc(F)cc3)c2c1N ; COCCCS(=O)c1sc2nc(-c3cccnc3)cc(-c3cn(C)cn3)c2c1N ; COCCCS(=O)c1sc2nc(-c3ccc(N)nc3)cc(-c3cccnc3)c2c1N]

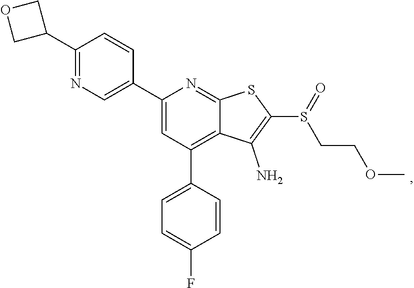 [SMILES: COCCS(=O)c1sc2nc(-c3ccc(C4COC4)nc3)cc(-c3ccc(F)cc3)c2c1N]

and pharmaceutically acceptable salts thereof.

In other embodiments, the 15-PGDH inhibitor can include a compound having a structure according to formula (IV):

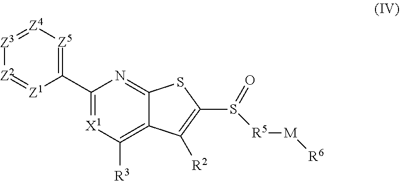

wherein n=0-2;
X¹ is N or CR^{a};
Z¹, Z², Z³, Z⁴, and Z⁵ are each independently N or CR^{b}, provided no more than 3 of Z¹, Z², Z³, Z⁴, and Z⁵ are N;
R² is selected from the group consisting of H, linear or branched alkyl, OH, Cl, F, NH₂, N(R⁷⁶)₂, and OR⁷⁷;
R³ is selected from the group consisting of:

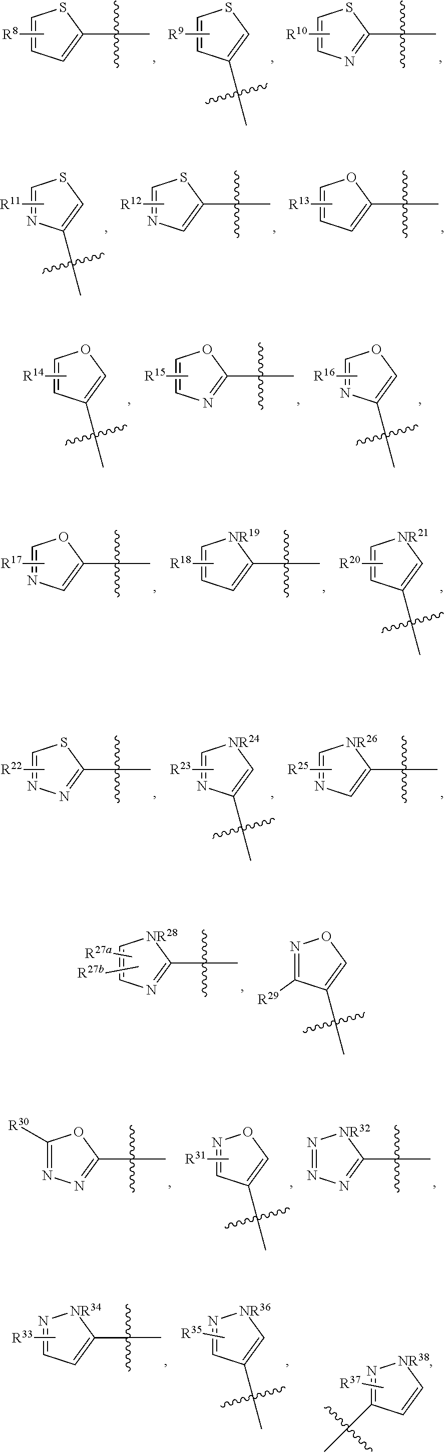

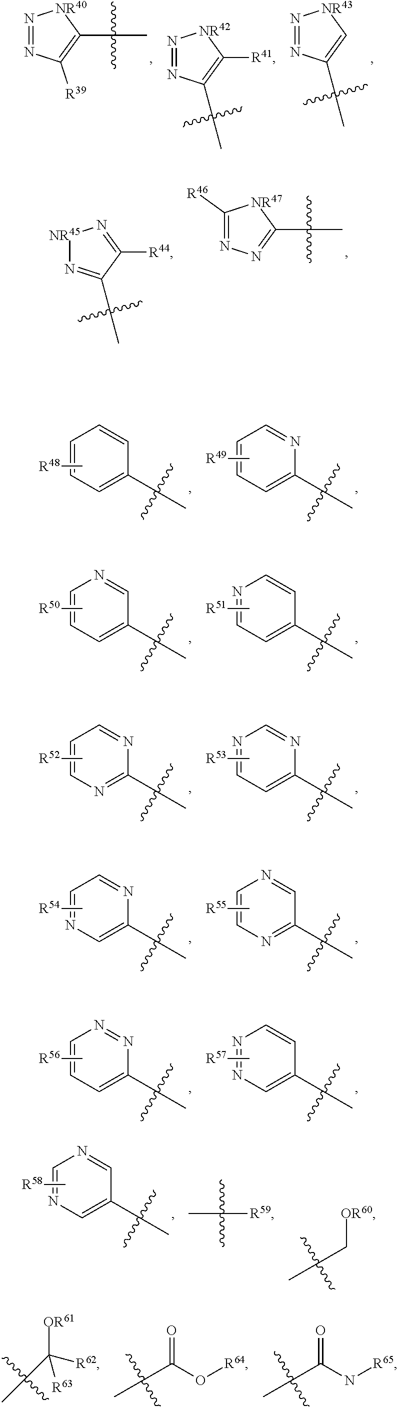

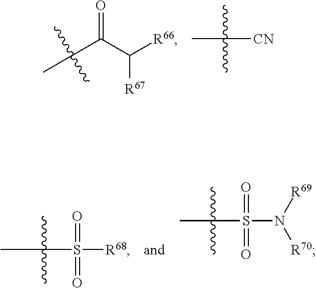

each R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷⁶, R⁷⁷, R^{a}, and R^{b} are the same or different and are independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 4-6 ring atoms, (wherein from 1-3 of the ring atoms is independently selected from N, NH, N(C₁-C₆ alkyl), NC(O)(C₁-C₆ alkyl), O, and S), heteroaryl or heterocyclyl containing from 4-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano(—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R═hydrogen, alkyl, aryl, alkaryl, aralkyl, etc.), arylimino (—CR═N(aryl), where R═hydrogen, alkyl, aryl, alkaryl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, arlyl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R═H, methyl or other alkyl], groups incorporating amino acids or other moieties expected to bear positive or negative charge at physiological pH, and combinations thereof;
M is O or S;
R⁵ and R⁶ are each independently C₁-C₂₄ alkylene;
and pharmaceutically acceptable salts thereof.

In some embodiments, R⁵ is a C₁-C₆ alkylene.

In other embodiments, R⁶ is a C₁-C₆ alkylene.

In some embodiments, R⁵ is a C₂-C₅ alkylene and R⁶ is a C₁-C₃ alkylene.

In other embodiments, M is O.

In still other embodiments, Ra is H.

In other embodiments, R² is NH².

In some embodiments, R³ is

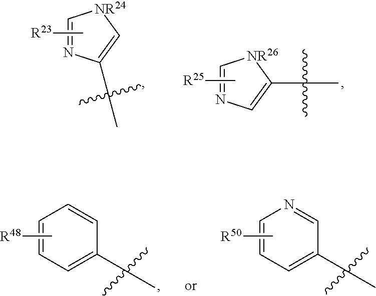

In other embodiments, each R²³, R²⁴, R²⁴, R²⁵, R⁴⁸, and R⁵⁰ are the same or different and independently selected hydrogen, halo, or C₁-C₃ alkyl.

In other embodiments, n=1.

Examples of compounds having formula (IV) are selected from the group consisting of:

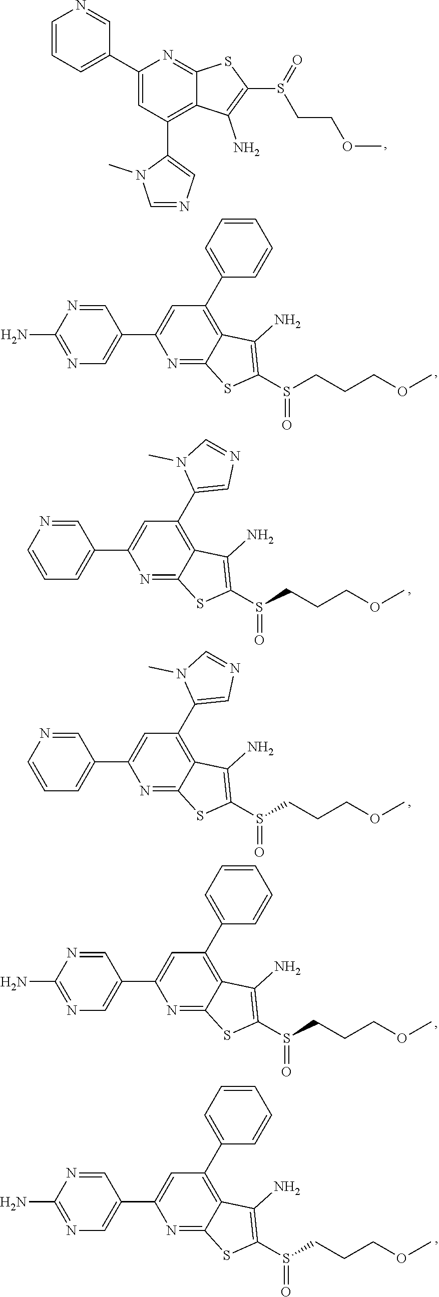

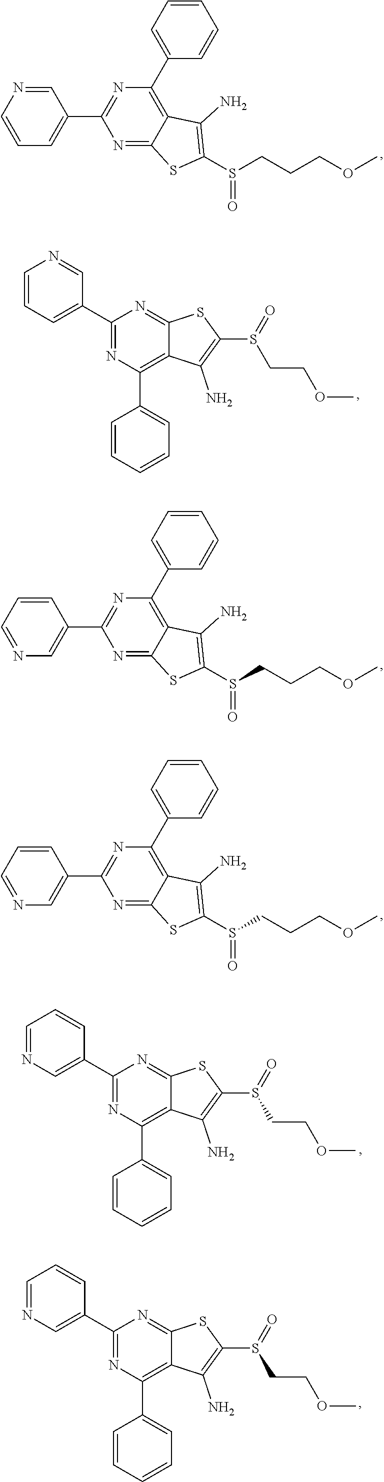 [SMILES: COCCCS(=O)c1sc2nc(-c3cccnc3)nc(-c3ccccc3)c2c1N ; COCCS(=O)c1sc2nc(-c3cccnc3)nc(-c3ccccc3)c2c1N ; COCCC[S@@](=O)c1sc2nc(-c3cccnc3)nc(-c3ccccc3)c2c1N ; COCCC[S@](=O)c1sc2nc(-c3cccnc3)nc(-c3ccccc3)c2c1N ; COCC[S@@](=O)c1sc2nc(-c3cccnc3)nc(-c3ccccc3)c2c1N ; COCC[S@](=O)c1sc2nc(-c3cccnc3)nc(-c3ccccc3)c2c1N]

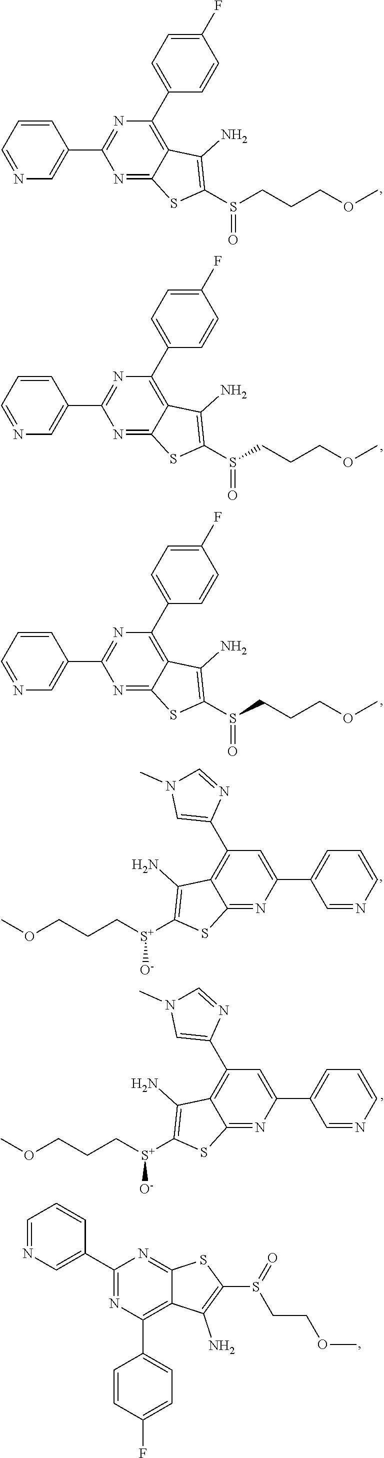

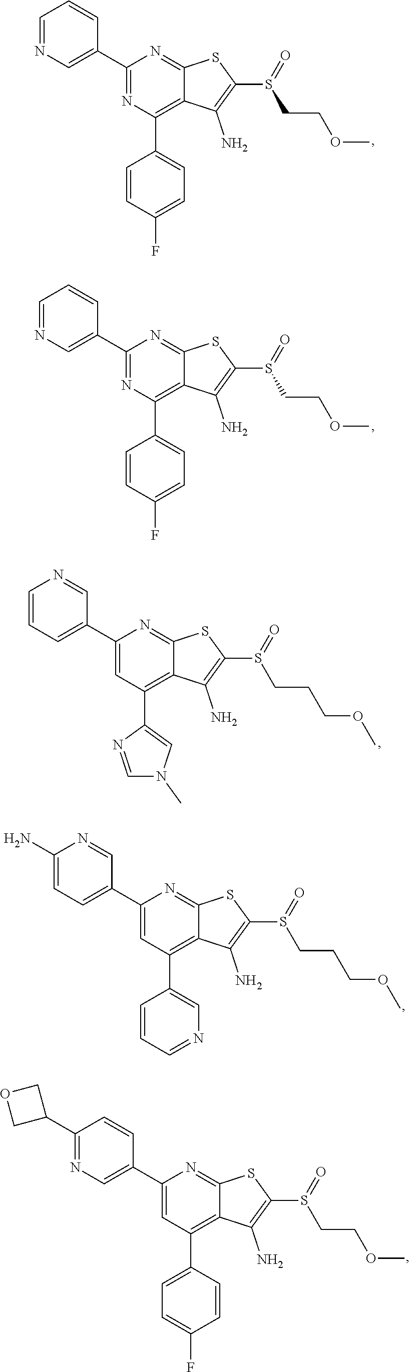 [SMILES: COCC[S@](=O)c1sc2nc(-c3cccnc3)nc(-c3ccc(F)cc3)c2c1N ; COCC[S@@](=O)c1sc2nc(-c3cccnc3)nc(-c3ccc(F)cc3)c2c1N ; COCCCS(=O)c1sc2nc(-c3cccnc3)cc(-c3cn(C)cn3)c2c1N ; COCCCS(=O)c1sc2nc(-c3ccc(N)nc3)cc(-c3cccnc3)c2c1N ; COCCS(=O)c1sc2nc(-c3ccc(C4COC4)nc3)cc(-c3ccc(F)cc3)c2c1N]

and pharmaceutically acceptable salts thereof.

In other embodiments, the 15-PGDH inhibitor can include a compound having the following structures:

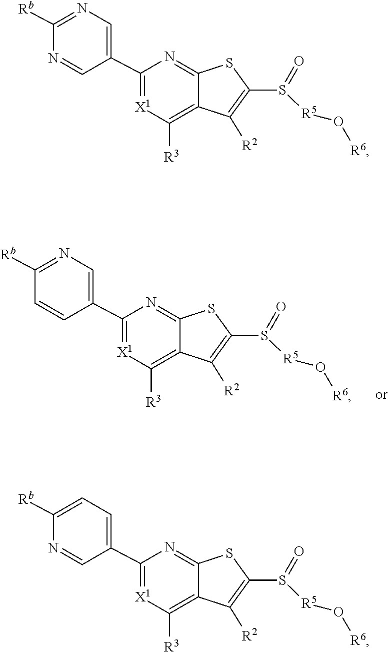

wherein:
R³ is

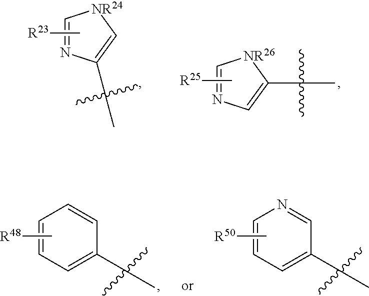

R⁵ is a C₂-C₅ alkyl and R⁶ is a C₁-C₃ alkylene;
each R²³, R²⁴, R²⁴, R²⁵, R⁴⁸, and R⁵⁰ are the same or different and independently selected hydrogen, halo, or C₁-C₃ alkyl;
each R^{b} is the same or different and independently selected from hydrogen, —NH₂, or heterocyclyl containing from 4-6 ring atoms (wherein 1 atom of the ring atoms is independently selected from O);
and pharmaceutically acceptable salts thereof.

In still other embodiments, at least one of R⁸—R⁷⁶ can independently be a group that improves aqueous solubility, for example, a phosphate ester (—OPO₃H₂), a phenyl ring linked to a phosphate ester (—OPO₃H₂), a phenyl ring substituted with one or more methoxyethoxy groups, or a morpholine, or an aryl or heteroaryl ring substituted with such a group.

In certain embodiments, the 15-PGDH inhibitor having formula (I-IV), can be selected that can ia) at 2.5 μM concentration, stimulate a Vaco503 reporter cell line expressing a 15-PGDH luciferase fusion construct to a luciferase output level of greater than 70 (using a scale on which a value of 100 indicates a doubling of reporter output over baseline); iia) at 2.5 μM concentration stimulate a V9m reporter cell line expressing a 15-PGDH luciferase fusion construct to a luciferase output level of greater than 75; iiia) at 7.5 μM concentration stimulate a LS174T reporter cell line expressing a 15-PGDH luciferase fusion construct to a luciferase output level of greater than 70; and iva) at 7.5 μM concentration, does not activate a negative control V9m cell line expressing TK-Renilla luciferase reporter to a level greater than 20; and va) inhibits the enzymatic activity of recombinant 15-PGDH protein at an IC₅₀ of less than 1 μM.

In other embodiments, the 15-PGDH inhibitor can ib) at 2.5 μM concentration, stimulate a Vaco503 reporter cell line expressing a 15-PGDH luciferase fusion construct to increase luciferase output; iib) at 2.5 μM concentration stimulate a V9m reporter cell line expressing a 15-PGDH luciferase fusion construct to increase luciferase output; iiib) at 7.5 μM concentration stimulate a LS174T reporter cell line expressing a 15-PGDH luciferase fusion construct to increase luciferase output; ivb) at 7.5 μM concentration, does not activate a negative control V9m cell line expressing TK-Renilla luciferase reporter to a luciferase level greater than 20% above background; and vb) inhibits the enzymatic activity of recombinant 15-PGDH protein at an IC₅₀ of less than 1 μM.

In other embodiments, the 15-PGDH inhibitor can inhibit the enzymatic activity of recombinant 15-PGDH at an IC₅₀ of less than 1 μM, or preferably at an IC₅₀ of less than 250 nM, or more preferably at an IC₅₀ of less than 50 nM, or more preferably at an IC₅₀ of less than 10 nM, or more preferably at an IC₅₀ of less than 5 nM at a recombinant 15-PGDH concentration of about 5 nM to about 10 nM.

In other embodiments, the 15-PGDH inhibitor can increase the cellular levels of PGE-2 following stimulation of an A459 cell with an appropriate agent, for example IL1β.

The 15-PGDH inhibitors described herein can be used for the prevention or the treatment of diseases that are associated with 15-PGDH and/or decreased prostaglandin levels and/or where it desirable to increase prostaglandin levels in the subject. For example, as discussed above, it is known that prostaglandins play an important role in hair growth. Specifically, internal storage of various types (A₂, F₂ₐ, E₂) of prostaglandins in the various compartments of hair follicles or their adjacent skin environments has been shown to be essential in maintaining and increasing hair density (Colombe L et. al, 2007, Exp. Dermatol, 16(9), 762-9). It has been reported that 15-PGDH, which is involved in the degradation of prostaglandins is present in the hair follicle dermal papillae, inactivates prostaglandins, especially, PGF₂ₐ and PGE₂, to cause scalp damage and alopecia (Michelet J F et. al., 2008, Exp. Dermatol, 17(10), 821-8). Thus, the compounds described herein, which have a suppressive or inhibitory activity against 15-PGDH that degrades prostaglandins, can improve scalp damage, prevent alopecia and promote hair growth and be used in a pharmaceutical composition for the prevention of alopecia and the promotion of hair growth.

In other embodiments, the 15-PGDH inhibitors described herein can be used in a pharmaceutical composition for promoting and/or inducing and/or stimulating pigmentation of the skin and/or skin appendages, and/or as an agent for preventing and/or limiting depigmentation and/or whitening of the skin and/or skin appendages, in particular as an agent for preventing and/or limiting canities.

In some embodiments, the 15-PGDH inhibitor can be applied to skin of a subject, e.g., in a topical application, to promote and/or stimulate pigmentation of the skin and/or hair growth, inhibit hair loss, and/or treat skin damage or inflammation, such as skin damage caused by physical or chemical irritants and/or UV-exposure.

In still other embodiments, the 15-PGDH inhibitors described herein can be used in a pharmaceutical composition for the prevention or the treatment of cardiovascular disease and/or diseases of vascular insufficiency, such as Raynaud's disease, Buerger's disease, diabetic neuropathy, and pulmonary artery hypertension. Prostaglandins including prostaglandin homologues produced in the body have been known to maintain the proper action of the blood vessel wall, especially to contribute to vasodilation for blood flow, preventing platelet aggregation and modulating the proliferation of smooth muscle that surrounds blood vessel walls (Yan. Cheng et. al., 2006, J. Clin., Invest). In addition, the inhibition of prostaglandins production or the loss of their activity causes the degeneration of the endothelium in the blood vessel walls, platelet aggregation and the dysfunction of cellular mechanism in the smooth muscle. Among others, the production of prostaglandins in blood vessels was shown to be decreased in hypertension patients, including pulmonary artery hypertension.

In other embodiments, the 15-PGDH inhibitors described herein can be used in a pharmaceutical composition for the prevention or the treatment of oral, intestinal, and/or gastrointestinal injury or diseases, or inflammatory bowel disease, such as oral ulcers, gum disease, gastritis, colitis, ulcerative colitis, and gastric ulcers. Gastritis and gastric ulcer, representatives of the gastrointestinal diseases, are defined as the conditions where gastrointestinal mucus membrane is digested by gastric acid to form ulcer. In the stomach walls generally consisting of mucosa, submucosa, muscle layer and serosa, gastric ulcer even damages submucosa and muscle layer, while gastritis damages mucosa only. Although the morbidity rates of gastritis and gastric ulcer are relatively high, the causes thereof have not been clarified yet. Until now, they are known to be caused by an imbalance between aggressive factors and defensive factors, that is, the increase in aggressive factors such as the increase in gastric acid or pepsin secretion, or the decrease in defensive factors such as structural or morphological deficit of the gastric mucus membrane, the decrease in mucus and bicarbonate ion secretion, the decrease in prostaglandin production, or the like.

Currently available therapeutic agents for gastritis and gastric ulcer comprise various drugs for strengthening the defensive factors such as an antacid, which does not affect, gastric acid secretion but neutralizes gastric acid that has been already produced, an inhibitor of gastric acid secretion, a promoter of prostaglandin secretion, and a coating agent for stomach walls. Especially, prostaglandins are known to be essential in maintaining the mechanism for protecting and defending gastric mucus membrane (Wallace J L., 2008, Physiol Rev., 88(4), 1547-65, S. J. Konturek et al., 2005, Journal of Physiology and Pharmacology, 56(5)). In view of the above, since the 15-PGDH inhibitors described herein show a suppressive or inhibitory activity against 15-PGDH, which degrades prostaglandins that protect gastric mucus membrane, they can be effective for the prevention or the treatment of gastrointestinal diseases, inter alia, gastritis and gastric ulcer.

Moreover, 15-PGDH inhibitors would also be expected to protect from other form of intestinal injury that would include toxicity from radiation, toxicity from chemotherapy, and chemotherapy induced mucositis.

In the kidney, prostaglandins modulate renal blood flow and may serve to regulate urine formation by both renovascular and tubular effects. In clinical studies, PGE₁ has been used to improve creatinine clearance in patients with chronic renal disease, to prevent graft rejection and cyclosporine toxicity in renal transplant patients, to reduce the urinary albumin excretion rate and N-acetyl-beta-D-glucosaminidase levels in patients with diabetic nephropathy (see Porter, Am., 1989, J. Cardiol., 64: 22E-26E). In addition, U.S. Pat. No. 5,807,895 discloses a method of preventing renal dysfunction by intravenous administration of prostaglandins such as PGE₁, PGE₂ and PGI₂. Furthermore, it has been reported that prostaglandins serve as vasodilators in the kidney, and, thus, the inhibition of prostaglandin production in the kidney results in renal dysfunction (Hao. C M, 2008, Annu Rev Physiol, 70, 357.about.77).

Thus, the 15-PGDH inhibitors described herein, which have a suppressive or inhibitory activity against 15-PGDH that degrades prostaglandins, may be effective in the prevention or the treatment of renal diseases that are associated with renal dysfunction.

The term “renal dysfunction” as used herein includes such manifestations as follows: lower than normal creatinine clearance, lower than normal free water clearance, higher than normal blood urea, nitrogen, potassium and/or creatinine levels, altered activity of kidney enzymes such as gamma glutamyl synthetase, alanine phosphatidase, N-acetyl-β-D-glucosaminidase, or β-ω-microglobulin; and increase over normal levels of macroalbuminuria.

Prostaglandins including PGE₁, PGE₂ and PGF₂ₐ have also been shown to stimulate bone resorption and bone formation to increase the volume and the strength of the bone (H. Kawaguchi et. al., Clinical Orthop. Rel. Res., 313, 1995; J. Keller et al., Eur. Jr. Exp. Musculoskeletal Res., 1, 1992, 8692). Considering that 15-PGDH inhibits the activities of prostaglandins as mentioned in the above, the inhibition of 15-PGDH activity may lead to the promotion of bone resorption and bone formation that are inhibited by 15-PGDH. Thus, the 15-PGDH inhibitors described herein can be effective for the promotion of bone resorption and bone formation by inhibiting 15-PGDH activity. 15-PGDH inhibitors can also be used to increase bone density, treat osteoporosis, promote healing of fractures, or promote healing after bone surgery or joint replacement, or to promote healing of bone to bone implants, bone to artificial implants, dental implants, and bone grafts.

In yet other embodiments, the 15-PGDH inhibitors described herein can effective for treating 15-PGDH expressing cancers. Inhibition of 15-PGDH can inhibit the growth, proliferation, and metastasis of 15-PGDH expressing cancers.

In still other embodiments, the 15-PGDH inhibitors described herein can be effective for wound healing. Among various prostaglandins, PGE₂ is known to serve as a mediator for wound healing. Therefore, when skin is injured by wounds or burns, the inhibition of 15-PGDH activity can produce the treatment effect of the wounds or the burns by PGE₂.

Additionally, as discussed above, increased prostaglandin levels have been shown to stimulate signaling through the Wnt signaling pathway via increased beta-catenin mediated transcriptional activity. Wnt signaling is known to be a key pathway employed by tissue stem cells. Hence, 15-PGDH inhibitors described herein may be utilized to increase tissue stem cell numbers for purposes that would include promoting tissue regeneration or repair in organs that would include liver, colon, and bone marrow. In addition, 15-PGDH inhibitors described herein may be utilized to promote tissue regeneration or repair in additional organs that would include but are not limited to brain, eye, cornea, retina, lung, heart, stomach, small intestine, pancreas, beta-cells of the pancreas, kidney, bone, cartilage, peripheral nerve.

Syndromic conditions, traumatic injuries, chronic conditions, medical interventions, or other conditions that cause or are associated with tissue damage and a need for tissue repair, and thus, suitable for treatment or amelioration using the methods described herein, include, but are not limited to, acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetes, diabetic neuropathy, diabetes induced ischemia, disseminated intravascular coagulation (DIC), embolic brain ischemia, graft-versus-host disease, frostbite, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular disease of the kidney, vascular inflammatory conditions, von Hippel-Lindau syndrome, and wounds to tissues or organs.

Other illustrative examples of genetic disorders, syndromic conditions, traumatic injuries, chronic conditions, medical interventions, or other conditions that cause or are associated with tissue damage and a need for tissue repair suitable for treatment or amelioration using the methods of the present invention, include, ischemia resulting from surgery, chemotherapy, radiation therapy, or cell, tissue, or organ transplant or graft.

In various embodiments, the methods of the invention are suitable for treating cerebrovascular ischemia, myocardial ischemia, limb ischemia (CLI), myocardial ischemia (especially chronic myocardial ischemia), ischemic cardiomyopathy, cerebrovascular ischemia, renal ischemia, pulmonary ischemia, intestinal ischemia, and the like.

In some embodiments, the ischemia is associated with at least one of acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetic neuropathy, diabetes induced ischemia, disseminated intravascular coagulation (DIC), embolic brain ischemia, graft-versus-host disease, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient isc hemic attack (TIA), traumatic brain injury, ulcerative colitis, vascular disease of the kidney, vascular inflammatory conditions, von Hippel-Lindau syndrome, and wounds to tissues or organs.

In some embodiments, the 15-PGDH inhibitor can be administered to a preparation of hematopoietic stem cells, such as peripheral blood hematopoietic stem cells or umbilical cord stem cells of the subject, to increase the fitness of the stem cell preparation as a donor graft or to decrease the number of units of umbilical cord blood required for transplantation.

Hematopoietic stem cells are multipotent stem cells that give rise to all the blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells), and others known in the art (See Fei, R., et al, U.S. Pat. No. 5,635,387; McGlave, et al, U.S. Pat. No. 5,460,964; Simmons, P., et al, U.S. Pat. No. 5,677,136; Tsukamoto, et al, U.S. Pat. No. 5,750,397; Schwartz, et al, U.S. Pat. No. 5,759,793; DiGuisto, et al, U.S. Pat. No. 5,681,599; Tsukamoto, et al, U.S. Pat. No. 5,716,827). Hematopoietic stem cells (HSCs) give rise to committed hematopoietic progenitor cells (HPCs) that are capable of generating the entire repertoire of mature blood cells over the lifetime of an organism.

Hematopoietic stem cells and hematopoietic progenitor cells are described herein generally as hematopoietic stem cells unless noted otherwise and can refer to cells or populations identified by the presence of the antigenic marker CD34 (CD34⁺). In some embodiments, the hematopoietic stem cells can be identified by the presence of the antigenic marker CD34 and the absence of lineage (lin) markers and are therefore characterized as CD34⁺/lin⁻ cells.

The hematopoietic stem cells used in the methods described herein may be obtained from any suitable source of hematopoietic stem and progenitor cells and can be provided as a high purified population of hematopoietic stem cells or as composition that includes about 0.01% to about 100% of hematopoietic stem cells. For example, hematopoietic stem cells may be provided in compositions, such as unfractionated bone marrow (where the hematopoiectic stem cells comprise less than about 1% of the bone marrow cell population), umbilical cord blood, placental blood, placenta, fetal blood, fetal liver, fetal spleen, Wharton's jelly, or mobilized peripheral blood.

Suitable sources of hematopoietic stem cells can be isolated or obtained from an organ of the body containing cells of hematopoietic origin. The isolated cells can include cells that are removed from their original environment. For example, a cell is isolated if it is separated from some or all of the components that normally accompany it in its native state. For example, an “isolated population of cells,” an “isolated source of cells,” or “isolated hematopoietic stem cells” and the like, as used herein, refer to in vitro or ex vivo separation of one or more cells from their natural cellular environment, and from association with other components of the tissue or organ, i.e., it is not significantly associated with in vivo substances.

Hematopoiectic stem cells can be obtained or isolated from bone marrow of adults, which includes femurs, hip, ribs, sternum, and other bones. Bone marrow aspirates containing hematopoiectic stem cells can be obtained or isolated directly from the hip using a needle and syringe. Other sources of hematopoietic stem cells include umbilical cord blood, placental blood, mobilized peripheral blood, Wharton's jelly, placenta, fetal blood, fetal liver, or fetal spleen. In particular embodiments, harvesting a sufficient quantity of hematopoietic stem cells for use in therapeutic applications may require mobilizing the stem and progenitor cells in the donor.

“Hematopoietic stem cell mobilization” refers to the release of stem cells from the bone marrow into the peripheral blood circulation for the purpose of leukapheresis, prior to stem cell transplantation. By increasing the number of stem cells harvested from the donor, the number of stem cells available for therapeutic applications can be significantly improved. Hematopoietic growth factors, e.g., granulocyte colony stimulating factor (G-CSF) or chemotherapeutic agents often are used to stimulate the mobilization. Commercial stem cell mobilization drugs exist and can be used in combination with G-CSF to mobilize sufficient quantities of hematopoietic stem and progenitor cells for transplantation into a subject. For example, G-CSF and Mozobil (Genzyme Corporation) can be administered to a donor in order to harvest a sufficient number of hematopoietic cells for transplantation. Other methods of mobilizing hematopoietic stem cells would be apparent to one having skill in the art.

In some embodiments, hematopoietic stem and progenitor cells (HSPCs) are obtained from umbilical cord blood. Cord blood can be harvested according to techniques known in the art {see, e.g., U.S. Pat. Nos. 7,147,626 and 7,131,958, herein incorporated by reference for such methodologies).

In one embodiment, HSPCs can be obtained from pluripotent stem cell sources, e.g., induced pluripotent stem cells (iPSCs) and embryonic stem cells (ESCs). As used herein, the term “induced pluripotent stem cell” or “iPSC” refers to a non-pluripotent cell that has been reprogrammed to a pluripotent state. Once the cells of a subject have been reprogrammed to a pluripotent state, the cells can then be programmed to a desired cell type, such as a hematopoietic stem or progenitor cell. As used herein, the term “reprogramming” refers to a method of increasing the potency of a cell to a less differentiated state. As used herein, the term “programming” refers to a method of decreasing the potency of a cell or differentiating the cell to a more differentiated state.

In some embodiments, the hematopoietic stem cells can be administered or contacted ex vivo with one or more 15-PGDH inhibitors described herein to provide a therapeutic composition. In one embodiment, the therapeutic compositions of the can include a population of hematopoietic stem cells treated ex vivo with a one or more 15-PGDH inhibitor. In certain embodiments, the therapeutic composition comprising the enhanced HSPCs is whole bone marrow, umbilical cord blood, or mobilized peripheral blood.

In particular embodiments, the therapeutic composition includes a population of cells, wherein the population of cells is about 95% to about 100% hematopoietic stem cells. The invention contemplates, in part, that using therapeutic compositions of highly purified hematopoietic stem cells, e.g., a composition comprising a population of cells wherein the cells comprise about 95% hematopoietic stem cells, may improve the efficiency of stem cell therapies. Currently practiced methods of transplantations typically use unfractionated mixtures of cells where hematopoietic stem cells comprise less than 1% of the total cell population.

In some embodiments, the therapeutic composition comprises a population of cells, wherein the population of cells comprises less than about 0.1%, 0.5%, 1%, 2%, 5%, 10%, 15%, 20%, 25%, or 30% hematopoietic stem cells. The population of cells in some embodiments comprises less than about 0.1%, 0.5%, 1%, 2%, 5%, 10%, 15%, 20%, 25%, or 30% hematopoietic stem cells. In other embodiments, the population of cells is about 0.1% to about 1%, about 1% to about 3%, about 3% to about 5%, about 10%-15%, about 15%-20%, about 20%-25%, about 25%-30%, about 30%-35%, about 35%-40%, about 40%-45%, about 45%-50%, about 60%-70%, about 70%-80%, about 80%-90%, about 90%-95%, or about 95% to about 100% hematopoietic stem cells.

Hematopoietic stem cells in the therapeutic compositions of the invention can be autologous/autogeneic (“self) or non-autologous (“non-self,” e.g., allogeneic, syngeneic or xenogeneic) relative to a subject to which the therapeutic composition is to be administered. “Autologous.” as used herein, refers to cells from the same subject. “Allogeneic.” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison.

Hematopoietic stem cells for use in the methods of the present invention may be depleted of mature hematopoietic cells such as T cells, B cells, NK cells, dendritic cells, monocytes, granulocytes, erythroid cells, and their committed precursors from bone marrow aspirate, umbilical cord blood, or mobilized peripheral blood (mobilized leukapheresis product). Mature, lineage committed cells are depleted by immunodepletion, for example, by labeling solid substrates with antibodies that bind to a panel of so-called “lineage” antigens: CD2, CD3, CD11b, CD14, CD15, CD16, CD79, CD56, CD123, and CD235a. A subsequent step can be performed to further purify the population of cells, in which a substrate labeled with antibodies that bind to the CD34⁺ antigen are used to isolate primitive hematopoietic stem cells. Kits are commercially available for purifying stem and progenitor cells from various cell sources and in particular embodiments, these kits are suitable for use with the methods described herein.

In one embodiment, the amount of hematopoietic stem cells in the therapeutic composition is at least 0.1×10⁵ cells, at least 0.5×10⁵ cells, at least 1×10⁵ cells, at least 5×10⁵ cells, at least 10×10⁵ cells, at least 0.5×10⁶ cells, at least 0.75×10⁶ cells, at least 1×10⁶ cells, at least 1.25×10⁶ cells, at least 1.5×10⁶ cells, at least 1.75×10⁶ cells, at least 2×10⁶ cells, at least 2.5×10⁶ cells, at least 3×10⁶ cells, at least 4×10⁶ cells, at least 5×10⁶ cells, at least 10×10⁶ cells, at least 15×10⁶ cells, at least 20×10⁶ cells, at least 25×10⁶ cells, or at least 30×10⁶ cells.

In one embodiment, the amount of hematopoietic stem cells in the therapeutic composition is the amount of HSPCs in a partial or single cord of blood, or is at least 0.1×10⁵ cells/kg of bodyweight, at least 0.5×10⁵ cells/kg of bodyweight, at least 1×10⁵ cells/kg of bodyweight, at least 5×10⁵ cells/kg of bodyweight, at least 10×10⁵ cells/kg of bodyweight, at least 0.5×10⁶ cells/kg of bodyweight, at least 0.75×10⁶ cells/kg of bodyweight, at least 1×10⁶ cells/kg of bodyweight, at least 1.25×10⁶ cells/kg of bodyweight, at least 1.5×10⁶ cells/kg of bodyweight, at least 1.75×10⁶ cells/kg of bodyweight, at least 2×10⁶ cells/kg of bodyweight, at least 2.5×10⁶ cells/kg of bodyweight, at least 3×10⁶ cells/kg of bodyweight, at least 4×10⁶ cells/kg of bodyweight, at least 5×10⁶ cells/kg of bodyweight, at least 10×10⁶ cells/kg of bodyweight, at least 15×10⁶ cells/kg of bodyweight, at least 20×10⁶ cells/kg of bodyweight, at least 25×10⁶ cells/kg of bodyweight, or at least 30×10⁶ cells/kg of bodyweight.

Preparations of hematopoietic stem cells administered one or more 15-PGDH inhibitors and/or therapeutic compositions that include hematopoietic stem cells and one or more 15-PGDH inhibitor can be used for improving hematopoietic stem cell transplants and in treating ischemia or ischemia-damaged tissue, and in reducing further damage to ischemic tissue and/or repairing damage to ischemic tissue through cell recruitment, improving vascularization in ischemic tissue, improving tissue regeneration at sites of ischemia, decreasing ischemic tissue necrosis or apoptosis, and/or increasing cell survival at sites of ischemia. In particular embodiments, the preparations of 15-PGDH inhibitor treated hematopoietic stem cells and/or therapeutic compositions of 15-PGDH inhibitors and hematopoietic stem cells are useful to subjects in need of hematopoietic reconstitution, such as subjects that have undergone or are scheduled to undergo myeloablative therapy.

Subjects, which can be treated with the preparations of 15-PGDH inhibitor treated hematopoietic stem cells and/or therapeutic compositions of 15-PGDH inhibitors and hematopoietic stem cells, can include subjects that have or that have been diagnosed with various types of leukemias, anemias, lymphomas, myelomas, immune deficiency disorders, and solid tumors. A subject also includes a human who is a candidate for stem cell transplant or bone marrow transplantation, such as during the course of treatment for a malignant disease or a component of gene therapy. Subjects may also include individuals or animals that donate stem cells or bone marrow for allogeneic transplantation. In certain embodiments, a subject may have undergone myeloablative irradiation therapy or chemotherapy, or may have experienced an acute radiation or chemical insult resulting in myeloablation. In certain embodiments, a subject may have undergone irradiation therapy or chemotherapy, such as during various cancer treatments. Typical subjects include animals that exhibit aberrant amounts (lower or higher amounts than a “normal” or “healthy” subject) of one or more physiological activities that can be modulated by an agent or a stem cell or marrow transplant.

Subjects, which can be treated with the preparations of 15-PGDH inhibitor treated hematopoietic stem cells and/or therapeutic compositions of 15-PGDH inhibitors and hematopoietic stem cells, can also include subjects undergoing chemotherapy or radiation therapy for cancer, as well as subjects suffering from (e.g., afflicted with) non malignant blood disorders, particularly immunodeficiencies (e.g. SCID, Fanconi's anemia, severe aplastic anemia, or congenital hemoglobinopathies, or metabolic storage diseases, such as Hurler's disease, Hunter's disease, mannosidosis, among others) or cancer, particularly hematological malignancies, such as acute leukemia, chronic leukemia (myeloid or lymphoid), lymphoma (Hodgkin's or non-Hodgkin's), multiple myeloma, myelodysplastic syndrome, or non-hematological cancers such as solid tumors (including breast cancer, ovarian cancer, brain cancer, prostate cancer, lung cancer, colon cancer, skin cancer, liver cancer, or pancreatic cancer).

Subjects may also include subjects suffering from aplastic anemia, an immune disorder (severe combined immune deficiency syndrome or lupus), myelodysplasia, thalassemaia, sickle-cell disease or Wiskott-Aldrich syndrome. In some embodiments, the subject suffers from a disorder that is the result of an undesired side effect or complication of another primary treatment, such as radiation therapy, chemotherapy, or treatment with a bone marrow suppressive drug, such as zidovadine, chloramphenical or gangciclovir. Such disorders include neutropenias, anemias, thrombocytopenia, and immune dysfunction. Other subjects may have disorders caused by an infection (e.g., viral infection, bacterial infection or fungal infection) which causes damage to stem or progenitor cells of the bone marrow.

In addition, subjects suffering from the following conditions can also benefit from treatment using the preparations of 15-PGDH inhibitor treated hematopoietic stem cells and/or therapeutic compositions of 15-PGDH inhibitors and hematopoietic stem cells: lymphocytopenia, lymphorrhea, lymphostasis, erythrocytopenia, erthrodegenerative disorders, erythroblastopenia, leukoerythroblastosis; erythroclasis, thalassemia, myelodysplasia, myelofibrosis, thrombocytopenia, disseminated intravascular coagulation (DIC), immune (autoimmune) thrombocytopenic purpura (ITP), HIV inducted ITP, myelodysplasia; thrombocytotic disease, thrombocytosis, congenital neutropenias (such as Kostmann's syndrome and Schwachman-Diamond syndrome), neoplastic associated neutropenias, childhood and adult cyclic neutropaenia; post-infective neutropaenia; myelodysplastic syndrome; neutropaenia associated with chemotherapy and radiotherapy; chronic granulomatous disease; mucopolysaccharidoses; Diamond Blackfan Anemia; Sickle cell disease; or Beta thalassemia major.

In other embodiments, the preparations of 15-PGDH inhibitor treated hematopoietic stem cells and/or therapeutic compositions or 15-PGDH inhibitors and hematopoietic stem cells can be used in cell-based therapy for treating ischemic tissue or treating or ameliorating one or more symptoms associated with tissue ischemia, including, but not limited to, impaired, or loss of, organ function (including without limitation impairments or loss of brain, kidney, or heart function), cramping, claudication, numbness, tingling, weakness, pain, reduced wound healing, inflammation, skin discoloration, and gangrene.

In one embodiment, the subject exhibits at least one symptom of an ischemic tissue or tissue damaged by ischemia. In particular embodiments, the subject is a human who is has or who is at risk of having an ischemic tissue or tissue damaged by ischemia, e.g., a subject that has diabetes, peripheral vascular disease, thromboangiitis obliterans, vasculitis, cardiovascular disease, coronary artery disease or heart failure, or cerebrovascular disease, cardiovascular disease, or cerebrovascular disease.

Illustrative examples of genetic disorders, syndromic conditions, traumatic injuries, chronic conditions, medical interventions, or other conditions that cause or are associated with ischemia, or increase the risk of ischemia in a subject, or cause a subject to exhibit more or more symptoms of ischemia, and thus, suitable for treatment or amelioration using the methods described herein, include, but are not limited to, acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetic neuropathy, diabetes induced ischemia, disseminated intravascular coagulation (DIC), embolic brain ischemia, graft-versus-host disease, frostbite, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular disease of the kidney, vascular inflammatory conditions, von Hippel-Lindau syndrome, and wounds to tissues or organs.

Other illustrative examples of genetic disorders, syndromic conditions, traumatic injuries, chronic conditions, medical interventions, or other conditions that cause or are associated with ischemia, or increase the risk of ischemia in a subject, or cause a subject to exhibit more or more symptoms of ischemia suitable for treatment or amelioration using the methods of the present invention, include, ischemia resulting from surgery, chemotherapy, radiation therapy, or cell, tissue, or organ transplant or graft.

In various embodiments, the methods of the invention are suitable for treating cerebrovascular ischemia, myocardial ischemia, limb ischemia (CLI), myocardial ischemia (especially chronic myocardial ischemia), ischemic cardiomyopathy, cerebrovascular ischemia, renal ischemia, pulmonary ischemia, intestinal ischemia, and the like.

In various embodiments, the invention contemplates that the therapeutic cell compositions disclosed herein can be used to treat an ischemic tissue in which it is desirable to increase the blood flow, oxygen supply, glucose supply, or supply of nutrients to the tissue.

In some embodiments, the 15-PGDH inhibitor can be administered to a preparation of tissue stem cells, such as neural stem stems, mesenchymal stem cells, or stem cells that can generate other tissues, and/or a preparation of pluripotent stem cells.

In one embodiment, tissue stems cells can be obtained from pluripotent stem cell sources, e.g., induced pluripotent stem cells (iPSCs) and embryonic stem cells (ESCs). As used herein, the term “induced pluripotent stem cell” or “iPSC” refers to a non-pluripotent cell that has been reprogrammed to a pluripotent state. Once the cells of a subject have been reprogrammed to a pluripotent state, the cells can then be programmed to a desired cell type, such as a hematopoietic stem or progenitor cell. As used herein, the term “reprogramming” refers to a method of increasing the potency of a cell to a less differentiated state. As used herein, the term “programming” refers to a method of decreasing the potency of a cell or differentiating the cell to a more differentiated state.

In some embodiments, the tissue stem cells and/or pluripotent stem cells can be administered or contacted ex vivo with one or more 15-PGDH inhibitors described herein to provide a therapeutic composition. In one embodiment, the therapeutic compositions of the can include a population of tissue stem cells treated ex vivo with a one or more 15-PGDH inhibitor.

In particular embodiments, the therapeutic composition includes a population of cells, wherein the population of cells is about 95% to about 100% tissue stem cells. The invention contemplates, in part, that using therapeutic compositions of highly purified tissue stem cells, e.g., a composition comprising a population of cells wherein the cells comprise about 95% tissue stem cells, may improve the efficiency of stem cell therapies.

In some embodiments, the therapeutic composition comprises a population of cells, wherein the population of cells comprises less than about 0.1%, 0.5%, 1%, 2%, 5%, 10%, 15%, 20%, 25%, or 30% tissue stem cells. The population of cells in some embodiments comprises less than about 0.1%, 0.5%, 1%, 2%, 5%, 10%, 15%, 20%, 25%, or 30% tissue stem cells. In other embodiments, the population of cells is about 0.1% to about 1%, about 1% to about 3%, about 3% to about 5%, about 10%-15%, about 15%-20%, about 20%-25%, about 25%-30%, about 30%-35%, about 35%-40%, about 40%-45%, about 45%-50%, about 60%-70%, about 70%-80%, about 80%-90%, about 90%-95%, or about 95% to about 100% tissue stem cells.

Tissue stem cells in the therapeutic compositions of the invention can be autologous/autogeneic (“self) or non-autologous (“non-self,” e.g., allogeneic, syngeneic or xenogeneic) relative to a subject to which the therapeutic composition is to be administered. “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison.

Preparations of tissue stem cells administered one or more 15-PGDH inhibitors and/or therapeutic compositions that include tissue stem cells and one or more 15-PGDH inhibitor can be used for improving tissue stem cell transplants and in treating damaged tissue, and in reducing further tissue damage tissue and/or potentiating repair to damaged tissue through stem cell recruitment and/or increasing cell survival at sites of tissue damage.

Syndromic conditions, traumatic injuries, chronic conditions, medical interventions, or other conditions that cause or are associated with tissue damage and a need for tissue repair, and thus, suitable for treatment or amelioration using the methods described herein, include, but are not limited to, acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetes, diabetic neuropathy, diabetes induced ischemia, disseminated intravascular coagulation (DIC), embolic brain ischemia, graft-versus-host disease, frostbite, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular disease of the kidney, vascular inflammatory conditions, von Hippel-Lindau syndrome, and wounds to tissues or organs.

Other illustrative examples of genetic disorders, syndromic conditions, traumatic injuries, chronic conditions, medical interventions, or other conditions that cause or are associated with tissue damage and a need for tissue repair suitable for treatment or amelioration using the methods of the present invention, include, ischemia resulting from surgery, chemotherapy, radiation therapy, or cell, tissue, or organ transplant or graft.

In various embodiments, the methods of the invention are suitable for treating cerebrovascular ischemia, myocardial ischemia, limb ischemia (CLI), myocardial ischemia (especially chronic myocardial ischemia), ischemic cardiomyopathy, cerebrovascular ischemia, renal ischemia, pulmonary ischemia, intestinal ischemia, and the like.

In other embodiments, the 15-PGDH inhibitor can be administered to a bone marrow graft donor or a hematopoietic stem cell donor to increase the fitness of a donor bone marrow graft or a donor hematopoietic stem cell graft.

In other embodiments, the 15-PGDH inhibitor can also be administered to bone marrow of a subject to increase stem cells in the subject or to increase the fitness of the marrow as a donor graft.

In yet other embodiments, the 15-PGDH inhibitor can be administered to a subject to mitigate bone marrow graft rejection, to enhance bone marrow graft engraftment, to enhance engraftment of a hematopoietic stem cell graft, or an umbilical cord blood stem cell graft, to enhance engraftment of a hematopoietic stem cell graft, or an umbilical cord stem cell graft, and/or to decrease the number of units of umbilical cord blood required for transplantation into the subject. The administration can be, for example, following treatment of the subject or the marrow of the subject with radiation therapy, chemotherapy, or immunosuppressive therapy.

In other embodiments, the 15-PGDH inhibitor can be administered to a recipient of a bone marrow transplant, of a hematopoietic stem cell transplant, or of an umbilical cord blood stem cell transplant, in order to decrease the administration of other treatments or growth factors.

In some embodiments, the 15-PGDH inhibitor can be administered to a subject to enhance recovery of neutrophils following bone marrow transplantation, following umbilical cord blood transplantation, following transplantation with hematopoietic stem cells, following conventional chemotherapy, following radiation treatment, and in individuals with neutropenias from diseases that include but are not limited to aplastic anemia, myelodysplasia, myelofibrosis, neutropenias from other bone marrow diseases, drug induced neutropenia, immune neutropenias, idiopathic neutropenia, and following infections with viruses that include, but are not limited to, HIV, CMV, and parvovirus.

In other embodiments, the 15-PGDH inhibitor can be administered to a subject to enhance recovery of platelets following bone marrow transplantation, following umbilical cord blood transplantation, following transplantation with hematopoietic stem cells, following conventional chemotherapy, following radiation treatment, and in individuals with neutropenias from diseases that include but are not limited to aplastic anemia, myelodysplasia, myelofibrosis, thrombocytopenias from other bone marrow diseases, drug induced thrombocytopenia, immune thrombocytopenia, idiopathic thrombocytopenic purpura, idiopathic thrombocytopenia, and following infections with viruses that include, but are not limited to, HIV, CMV, and parvovirus.

In still other embodiments, the 15-PGDH inhibitor can be administered to a subject to enhance recovery of hemoglobin following bone marrow transplantation, following umbilical cord blood transplantation, following transplantation with hematopoietic stem cells, following conventional chemotherapy, following radiation treatment, and in individuals with anemias from diseases that include but are not limited to aplastic anemia, myelodysplasia, myelofibrosis, anemia from other bone marrow diseases, drug induced anemia, immune mediated anemias, anemia of chronic disease, idiopathic anemia, and following infections with viruses that include, but are not limited to, HIV, CMV, and parvovirus.

In some embodiments, the 15-PGDH inhibitor can be administered to a subject to enhance numbers of bone marrow stem cell numbers following bone marrow transplantation, following umbilical cord blood transplantation, following transplantation with hematopoietic stem cells, following conventional chemotherapy, following radiation treatment, in individuals with other bone marrow diseases, in individuals with cytopenias following viral infections, and in individuals with cytopenias.

In other embodiments, the 15-PGDH inhibitor can be administered to a subject to enhance response to cytokines administered to individuals with cytopenias that include but are not limited to neutropenia, thrombocytopenia, lymphocytopenia, and anemia. Cytokines whose responses may be enhanced by SW033291 include, but are not limited to: G-CSF, GM-CSF, EPO, IL-3, IL-6, TPO, SCF, and TPO-RA (thrombopoietin receptor agonist).

In further embodiments, the 15-PGDH inhibitor can be administered to a subject or to a tissue graft of a subject to mitigate graft rejection, to enhance graft engraftment, to enhance graft engraftment following treatment of the subject or the marrow of the subject with radiation therapy, chemotherapy, or immunosuppressive therapy, to confer resistance to toxic or lethal effects of exposure to radiation, confer resistance to the toxic effect of Cytoxan, the toxic effect of fludarabine, the toxic effect of chemotherapy, or the toxic effect of immunosuppressive therapy, to decrease infection, and/or to decrease pulmonary toxicity from radiation.

In other embodiments, the 15-PGDH inhibitor can be administered to a recipient of a tissue stem cell transplant, including but not limited to a transplant with hematopoietic stem cells, neural stem stems, mesenchymal stem cells, or stem cells for other tissues, so as to accelerate tissue regeneration and repair following the transplant.

In some embodiments, the administration of a 15-PGDH inhibitor can be in combination with G-CSF for the purpose of increasing neutrophils.

In other embodiments, the administration of a 15-PGDH inhibitor can be in combination with a hematopoietic cytokine for the purpose of increasing neutrophils.

In still other embodiments, the administration of a 15-PGDH inhibitor can be in combination with G-CSF for the purpose of increasing numbers of and/or of mobilizing peripheral blood hematopoietic stem cells.

In other embodiments, the administration of a 15-PGDH inhibitor can be in combination with a hemopoietic cytokine for the purpose of increasing numbers of and/or of mobilizing peripheral blood hematopoietic stem cells.

In some embodiments, the administration of a 15-PGDH inhibitor can be in combination with a second agent, including Plerixafor, for the purpose of increasing numbers of and/or of mobilizing peripheral blood hematopoietic stem cells.

In other embodiments, the administration of a 15-PGDH inhibitor can be in combination with G-CSF for the purpose of increasing numbers of and/or of mobilizing peripheral blood hematopoietic stem cells for use in hematopoietic stem cell transplantation.

In still other embodiments, the administration of a 15-PGDH inhibitor can be in combination with a hemopoietic cytokine for the purpose of increasing numbers of and/or of mobilizing peripheral blood hematopoietic stem cells for use in hematopoietic stem cell transplantation.

In other embodiments, the administration of a 15-PGDH inhibitor can be in combination with a second agent, including Plerixafor, for the purpose of increasing numbers of and/or of mobilizing peripheral blood hematopoietic stem cells for use in hematopoietic stem cell transplantation.

In still other embodiments, the administration of a 15-PGDH inhibitor can be in combination with G-CSF for the purpose of increasing numbers of hematopoietic stem cells in blood or bone marrow.

In other embodiments, the administration of a 15-PGDH inhibitor can be in combination with a hemopoietic cytokine for the purpose of increasing numbers of hematopoietic stem cells in blood or bone marrow.

In other embodiments, the 15-PGDH inhibitors can be used to treat and/or prevent fibrosis and various fibrotic diseases, disorders or conditions, and decrease fibrotic symptoms, such as collagen deposition, inflammatory cytokine expression, and inflammatory cell infiltration.

In some embodiments, a method of treating or preventing a fibrotic disease, disorder or condition includes administering to a subject in need thereof a therapeutically effect amount of a 15-PGDH inhibitor such that at least one symptom or feature of a fibrotic disease, disorder or condition, or other related diseases, disorders or conditions, is reduced in intensity, severity, or frequency, or has delayed onset.

As used herein, the term “fibrotic” diseases, disorders, or conditions include diseases, disorders, or conditions characterized, in whole or in part, by the excess production of fibrous material, including excess production of fibrotic material within the extracellular matrix, or the replacement of normal tissue elements by abnormal, non-functional, and/or excessive accumulation of matrix-associated components. The fibriotic disesases, disorders, or conditions, can include acute and chronic, clinical or subclinical presentation, in which fibrogenic associated biology or pathology is evident.

Examples of fibrotic diseases, disorders and conditions include systemic sclerosis, multifocal fibrosclerosis, nephrogenic systemic fibrosis, scleroderma (including morphea, generalized morphea, or linear scleroderma), sclerodermatous graft-vs-host-disease, kidney fibrosis (including glomerular sclerosis, renal tubulointerstitial fibrosis, progressive renal disease or diabetic nephropathy), cardiac fibrosis (e.g., myocardial fibrosis), pulomanry fibrosis (e.g., glomerulosclerosis pulmonary fibrosis, idiopathic pulmonary fibrosis, silicosis, asbestosis, interstitial lung disease, interstitial fibrotic lung disease, and chemotherapy/radiation induced pulmonary fibrosis), oral fibrosis, endomyocardial fibrosis, deltoid fibrosis, pancreatitis, inflammatory bowel disease, Crohn's disease, nodular fascilitis, eosinophilic fasciitis, general fibrosis syndrome characterized by replacement of normal muscle tissue by fibrous tissue in varying degrees, retroperitoneal fibrosis, liver fibrosis, liver cirrhosis, chronic renal failure; myelofibrosis (bone marrow fibrosis), drug induced ergotism, glioblastoma in Li-Fraumeni syndrome, sporadic glioblastoma, myleoid leukemia, acute myelogenous leukemia, myelodysplastic syndrome, myeloproferative syndrome, gynecological cancer, Kaposi's sarcoma, Hansen's disease, collagenous colitis, acute fibrosis, organ specific fibrosis, and the like.

Illustrative organ specific fibrotic disorders include, but are not limited to, pulmonary fibrosis, pulmonary hypertension, cystic fibrosis, asthma, chronic obstructive pulmonary disease, liver fibrosis, kidney fibrosis, NASH, and the like. Many fibrotic diseases, disorders or conditions have disordered and/or exaggerated deposition of extracellular matrix in affected tissues. Fibrosis may be associated with inflammation, occur as a symptom of underlying disease, and/or caused by surgical procedure or wound healing process. Unchecked fibrosis can result in destruction of the architecture of the underlying organ or tissue, commonly referred to as scarring.

In some embodiments, the 15-PGDH inhibitors can be used to treat or prevent lung fibrosis. The lung fibrosis can be selected from the group consisting of pulmonary fibrosis, pulmonary hypertension, chronic obstructive pulmonary disease (COPD), asthma, idiopathic pulmonary fibrosis, sarcoidosis, cystic fibrosis, familial pulmonary fibrosis, silicosis, asbestosis, coal worker's pneumoconiosis, carbon pneumoconiosis, hypersensitivity pneumonitides, pulmonary fibrosis caused by inhalation of inorganic dust, pulmonary fibrosis caused by an infectious agent, pulmonary fibrosis caused by inhalation of noxious gases, aerosols, chemical dusts, fumes or vapors, drug-induced interstitial lung disease, or pulmonary hypertension, and combinations thereof.

Pulmonary fibrosis is characterized by progressive scarring of lung tissue accompanied by fibroblast proliferation, excessive accumulation of extracellular matrix proteins, and abnormal alveolar structure. The thickened and stiff tissue makes it difficult for lungs to work properly, leading to breathing problems such as shortness of breath, and can ultimately be fatal. Pulmonary fibrosis may be caused by acute lung injury, viral infection, exposure to toxins, radiation, chronic disease, medications, or may be idiopathic (i.e., an undiscovered underlying cause).

The classic findings in idiopathic pulmonary fibrosis show diffuse peripheral scarring of the lungs with small bubbles (known as bullae) adjacent to the outer lining of the surface of the lung, often at the bases of the lungs. Idiopathic pulmonary fibrosis often has a slow and relentless progression. Early on, patients often complain of a dry unexplained cough. Next, shortness of breath (dyspnea) sets in and worsens over time triggered by less and less activity. Eventually, the shortness of breath becomes disabling, limiting all activity and even occurring while sitting still. In rarer cases, the fibrosis can be rapidly progressive, with dyspnea and disability occurring in weeks to months of onset of the disease. This form of pulmonary fibrosis has been referred to as Hamman-Rich syndrome.

Pulmonary hypertension is marked by an increase in the blood pressure of the lung vasculature, including the pulmonary artery, pulmonary vein, and/or pulmonary capillaries. Abnormally high pressure strains the right ventricle of the heart, causing it to expand. Over time, the right ventricle can weaken and lose its ability to pump enough blood to the lungs, leading to the development of heart failure. Pulmonary hypertension can occur as a result of other medical conditions, such as chronic liver disease and liver cirrhosis; rheumatic disorders such as scleroderma or systemic lupus erythematosus (lupus); and lung conditions including tumors, emphysema, chronic obstructive pulmonary disease (COPD), and pulmonary fibrosis. Pulmonary fibrosis may lead to narrowing of pulmonary vasculature resulting in pulmonary hypertension.

Chronic Obstructive Pulmonary Disease (COPD) is a common lung disease that is often associated with chronic bronchitis or emphysema. Symptoms can often include cough, mucus build up, fatigue, wheezing, and respiratory infection.

Chronic bronchitis and emphysema are diseases of the lungs in which the airways become narrowed. This leads to a limitation of the flow of air to and from the lungs, causing shortness of breath (dyspnea). In clinical practice, COPD is defined by its characteristically low airflow on lung function tests.

Lung damage and inflammation in the large airways results in chronic bronchitis. In the airways of the lung, the hallmark of chronic bronchitis is an increased number (hyperplasia) and increased size (hypertrophy) of the goblet cells and mucous glands of the airway. As a result, there is more mucus than usual in the airways, contributing to narrowing of the airways and causing a cough with sputum. Microscopically there is infiltration of the airway walls with inflammatory cells. Inflammation is followed by scarring and remodeling that thickens the walls and also results in narrowing of the airways. As chronic bronchitis progresses, there is squamous metaplasia (an abnormal change in the tissue lining the inside of the airway) and fibrosis (further thickening and scarring of the airway wall). The consequence of these changes is a limitation of airflow and difficulty breathing.

Asthma is a chronic lung disease characterized by inflammation and constriction of the airways. Asthma causes recurring periods of wheezing, tightness of the chest, shortness of breath, and coughing. Swelling and overproduction of mucus can cause further airway constriction and worsening of symptoms. There is evidence that increased matrix degradation may occur in asthma, and this may contribute to mechanical changes in the airways in asthma (Roberts et al (1995) Chest 107:111 S-117S, incorporated herein by reference in its entirety. Treatment of extracellular matrix degradation may ameliorate symptoms of asthma.

Cystic fibrosis is a recessive multi-system genetic disease characterized by abnormal transport of chloride and sodium across epithelium, leading to thick, viscous secretions in the lungs, pancreas, liver, intestine and reproductive tract. Cystic fibrosis is caused by a mutation in the gene for the protein cystic fibrosis transmembrane conductance regulator (CFTR). Lung disease results from clogging of the airways due to mucus build-up, decreased mucociliary clearance, and resulting inflammation, which can cause fibrotic injury and structural changes to the lungs. The fibrotic lung damage progresses over time leading some cystic fibrosis patients to require lung transplant.

Common symptoms of subjects suffering from cystic fibrosis include, but are not limited to, accumulation of thick mucus, copious phlegm production, frequent chest infections, frequent coughing, frequent shortness of breath, inflammation, decreased ability to exercise, opportunistic infections of the lung and sinus (including but not limited to Staphylococcus aureus, Haemophilus influenzae, Mycobacterium aviium, and Pseudomonas aeruginosa), pneumonia, tuberculosis, bronchiectasis, hemoptysis, pulmonary hypertension (and resulting heart failure), hypoxia, respiratory failure, allergic bronchopulmonary aspergillosis, mucus in the paranasal sinuses, sinus infection, facial pain, fever, excessive nasal drainage, development of nasal polyps, cardiorespiratory complications, CF-related diabetes, rectal prolapse, pancreatitis, malabsorption, intestinal blockage, exocrine pancreatic insufficiency, bile duct blockage, and liver cirrhosis.

In other embodiments, the 15-PGDH inhibitors can be used to treat or prevent fibrotic diseases, disorders or conditions caused by post-surgical adhesion formation. Post-surgical adhesion formation is a common complication of surgery. The formation of adhesions, from mechanical damage, ischemia, and infections, can increase morbidity and mortality following surgery. Although refined surgical procedures can reduce the magnitude of adhesion formation, adhesions are rarely eviscerated and an effective adjunctive therapy is needed. Reducing the fibrosis associated with this process could reduce pain, obstruction and other complications of surgery and promote healing and recovery.

Wounds (i.e., lacerations, openings) in mammalian tissue result in tissue disruption and coagulation of the microvasculature at the wound face. Repair of such tissue represents an orderly, controlled cellular response to injury. Soft tissue wounds, regardless of size, heal in a similar manner. Tissue growth and repair are biologic systems wherein cellular proliferation and angiogenesis occur in the presence of an oxygen gradient. The sequential morphological and structural changes which occur during tissue repair have been characterized in detail and have in some instances been quantified (see e.g., Hunt, T. K., et al., “Coagulation and macrophage stimulation of angiogenesis and wound healing,” in The Surgical Wound, pp. 1-18, ed. F. Dineen & G. Hildrick-Smith (Lea & Febiger, Philadelphia: 1981)). The cellular morphology consists of three distinct zones. The central avascular wound space is oxygen deficient, acidotic and hypercarbic, and has high lactate levels. Adjacent to the wound space is a gradient zone of local anemia (ischemia) which is populated by dividing fibroblasts. Behind the leading zone is an area of active collagen synthesis characterized by mature fibroblasts and numerous newly-formed capillaries (i.e., neovascularization). U.S. Pat. Nos. 5,015,629 and 7,022,675 (each incorporated by reference herein) disclose methods and compositions for increasing the rate of wound repair.

In some embodiments, the 15-PGDH inhibitors can used for reducing or preventing scar formation in a subject by administering to a subject in need of treatment. Scar formation is a natural part of the healing process. Disorderly collagen synthesis and deposition in a wound can result in excessive, thick, or raised scar formation. Generally, the larger the wound, the longer it takes to heal and the greater the chance of a problematic scar.

In other embodiments, the 15-PGDH inhibitors can be used to reduce or prevent scar formation on skin or scleroderma. There are several types of scars on skin. Hypertropic scars are raised, pinkish-red areas located inside the borders of the original injury. They are often described as itchy. In some cases, hypertropic scars shrink and fade on their own. Keloids are raised, deep-red areas that tend to cover much more area than that of the original injury. Even when surgically removed, keloids tend to recur. Atrophic scars are skin depressions, like those that sometimes form from severe acne. They are caused by inflammation that destroys the collagen during the rebuilding process, leaving an area of indentation.

In some embodiments, the 15-PGDH inhibitors can be used to treat or prevent systemic sclerosis. Systemic sclerosis is a systemic connective tissue disease characterized by alterations of the microvasculature, disturbances of the immune system and by massive deposition of collagen and other matrix substances in the connective tissue. Systemic sclerosis is a clinically heterogeneous generalized disorder which affects the connective tissue of the skin and internal organs such as gastrointestinal tract, lungs, heart and kidneys. Reduction of fibrosis resulting from systemic sclerosis may ameliorate symptoms and/or prevent further complications in affected tissues.

In other embodiments, the 15-PGDH inhibitors can be used to treat or prevent liver fibrosis. Liver fibrosis can result from a chronic liver disease, viral induced hepatic cirrhosis, hepatitis B virus infection, hepatitis C virus infection, hepatitis D virus infection, schistosomiasis, primary biliary cirrhosis, alcoholic liver disease or non-alcoholic steatohepatitis (NASH), NASH associated cirrhosis obesity, diabetes, protein malnutrition, coronary artery disease, auto-immune hepatitis, cystic fibrosis, α-1-antitrypsin deficiency, primary biliary cirrhosis, drug reaction and exposure to toxins.

Nonalcoholic steatohepatitis (NASH) is a common liver disease. It resembles alcoholic liver disease but occurs in people who drink little or no alcohol. The major feature in NASH is fat in the liver, along with inflammation and damage. Nevertheless, NASH can be severe and can lead to cirrhosis, in which the liver is permanently damaged and scarred and no longer able to work properly.

NASH is usually a silent disease with few or no symptoms. Patients generally feel well in the early stages and only begin to have symptoms—such as fatigue, weight loss, and weakness—once the disease is more advanced or cirrhosis develops. The progression of NASH can take years, even decades. The process can stop and, in some cases may even begin to reverse on its own without specific therapy. Or NASH can slowly worsen, causing scarring or fibrosis to appear and accumulate in the liver. As fibrosis worsens, cirrhosis develops in which the liver becomes seriously scarred, hardened, and unable to function normally. Not every person with NASH develops cirrhosis, but once serious scarring or cirrhosis is present, few treatments can halt the progression. A person with cirrhosis experiences fluid retention, muscle wasting, bleeding from the intestines, and liver failure. Liver transplantation is the only treatment for advanced cirrhosis with liver failure, and transplantation is increasingly performed in people with NASH. NASH ranks as one of the major causes of cirrhosis in America, behind hepatitis C and alcoholic liver disease.

In some embodiments, the 15-PGDH inhibitors can be used to treat or prevent kidney fibrosis. Kidney fibrosis can result from dialysis following kidney failure, catheter placement, a nephropathy, glomerulosclerosis, glomerulonephritis, chronic renal insufficiency, acute kidney injury, end stage renal disease or renal failure.

Kidney (renal) fibrosis results from excessive formation of fibrous connective tissue in the kidney. Kidney fibrosis causes significant morbidity and mortality and leads to a need for dialysis or kidney transplantation. Fibrosis can occur in either the filtering or reabsorptive component of the nephron, the functional unit of the kidney. A number of factors may contribute to kidney scarring, particularly derangements of physiology involved in the autoregulation of glomerular filtration. This in turn leads to replacement of normal structures with accumulated extracellular matrix. A spectrum of changes in the physiology of individual cells leads to the production of numerous peptide and non-peptide fibrogens that stimulate alterations in the balance between extracellular matrix synthesis and degradation to favor scarring.

In some embodiments, the symptoms of fibrosis of a tissue organ can comprise inflammation. In these embodiments, a therapeutically effective amount of the 15-PGDH inhibitor administered to the subject in need thereof can be an amount effective to decrease or reduce inflammatory cell count in the tissue or organ. A relevant sample can be obtained from the subject to determine the decrease or reduction in inflammatory cell count. In a non-limiting embodiment, the beneficial effect may be assessed by demonstrating a reduction in neutrophil count in BAL fluid from the subject with cystic fibrosis. The excessive recruitment of neutrophils into the airways of patients with CF is a significant predictor of lung disease severity in CF and therefore is an important therapeutic target. Methods for measuring such cell counts are well known in the art, including but not limited to FACS techniques. In some embodiments, the method may comprise reducing neutrophil cell count in BAL fluid from the subject compared to control. Any suitable control can be used for comparison, such as cystic fibrosis subjects not treated the 15-PGDH inhibitors. In some embodiments, a decrease in inflammatory cell count, such as neutrophil count, provides a clinical benefit to the subject. In various embodiments, the reduction in inflammatory cell count is at least 5%, 10%, 15%, 20%, 25%, 50%, or more compared to control.

In another embodiment, the beneficial effect of the 15-PGDH inhibitors may be assessed by a reduction in one or more inflammatory biomarkers in a relevant sample from the subject. In various non-limiting embodiments, the inflammatory biomarker may comprise or consist of one or more of cytokines or inflammatory cytokines associated with fibrosis. Such cytokines can include, for example, IL1β, MIP2 (e.g., CCL3 or CCL4), IFNδ, TGFβ, TNFα, IL-6, MCP-1, IL2, and IL-10 in BAL fluid. Methods for measuring the amount of such biomarkers are well known in the art, including but not limited to ELISAs. Thus, in this embodiment, the methods may further comprise the reducing an amount of one or more inflammatory biomarkers in a sample from the subject compared to control.

In other embodiments, the 15-PGDH inhibitors can be used in a method for decreasing or reducing collagen secretion or collagen deposition in a tissue or organ, such as the lung, the liver, the skin or the heart, of a subject. The method can include administering a therapeutically effective amount of the 15-PGDH inhibitors to the subject in need thereof. The subject can have or be at risk of an excessive collagen secretion or collagen deposition in the tissue or organ, such as the kidney, the lung, the liver, the intestines, the colon, the skin or the heart. Usually, the excessive collagen secretion or collagen deposition in an organ results from an injury or an insult. Such injury and insult are organ-specific. The 15-PGDH inhibitors can be administered over a sufficient period of time to decrease or reduce the level of collagen deposition in the tissue or organ, completely or partially. A sufficient period of time can be during one week, or between 1 week to 1 month, or between 1 to 2 months, or 2 months or more. For chronic condition, the 15-PGDH inhibitors can be advantageously administered for life time period.

15-PGDH inhibitors used to treat the fibrotic disease, disorder or condition and/or reduce collagen deposition can be identified using assays in which putative inhibitor compounds are applied to cells expressing 15-PGDH and then the functional effects on 15-PGDH activity are determined. Samples or assays comprising 15-PGDH that are treated with a potential inhibitor are compared to control samples without the inhibitor to examine the extent of effect. Control samples (untreated with modulators) are assigned a relative 15-PGDH activity value of 100%. Inhibition of 15-PGDH is achieved when the 15-PGDH activity value relative to the control is about 80%, optionally 50% or 25%, 10%, 5% or 1%.

Additionally, in a model organism, PGE₂ signaling stimulates liver regeneration and increase survival after exposure to hepatoxic agents, such as acetaminophen. Hence, 15-PGDH inhibitors described herein may be utilized to increase liver regeneration after liver resection, in other settings that include after liver surgery, after live liver donation, or after receiving a liver transplant or to increase liver regeneration and increase survival after exposures to hepatoxic agents, including but not limited to acetaminophen and similar compounds.

PGE1 analogues have also been used in the treatment of erectile dysfunction. Accordingly, in some embodiments, 15-PGDH inhibitors described herein can used either alone or combination with a prostaglandin for the treatment of erectile dysfunction.

Other embodiments described herein relate to the use of 15-PGDH inhibitors in combination with corticosteroids to treat inflammation and/or reduce aberrant activity of the immune system in a subject in need thereof. It was found that corticosteroids administered to a subject can induce 15-PGDH expression in tissue of the subject. Administration of a 15-PGDH inhibitor in combination with a corticosteroid was found to enhance anti-inflammatory and/or immunosuppressive effects of the corticosteroid while attenuating corticosteroid induced adverse and/or cytotoxic effects. Treatment of inflammatory and/or immune disorders by administration of 15-PGDH inhibitors in combination with corticosteroids can increase therapeutic efficacy and can allow the corticosteroids to be administered, in some instances, at lower dosages to achieve similar effects, and, in other instances, at higher dosages and for prolonged periods of times with attenuated and/or reduced adverse or cytotoxic effects. Additional embodiments herein relate to the use of 15-PGDH inhibitors in combination with TNF alpha inhibitors to treat inflammation and/or reduce aberrant activity of the immune system in a subject in need thereof.

In some embodiments, the 15-PGDH inhibitors can be administered in combination with corticosteroids and/or TNF inhibitors to treat intestinal, gastrointestinal, or bowel disorders. The intestinal, gastrointestinal, or bowel disorders treated can include oral ulcers, gum disease, gastritis, colitis, ulcerative colitis, gastric ulcers, inflammatory bowel disease, and Crohn's disease. As described below, it was found that that inhibitors of short-chain dehydrogenase activity, such as 15-PGDH inhibitors, can be administered to a subject in need thereof alone or in combination with corticosteroids to treat intestinal, gastrointestinal, or bowel disorders, such as oral ulcers, gum disease, gastritis, colitis, ulcerative colitis, gastric ulcers, inflammatory bowel disease, and Crohn's disease.

The 15-PGDH inhibitors described herein can be used in a pharmaceutical composition for the prevention or the treatment of oral, intestinal, and/or gastrointestinal injury or diseases, or inflammatory bowel disease (IBD), such as Crohn's disease, oral ulcers, gum disease, gastritis, colitis, ulcerative colitis, and gastric ulcers. Gastritis and gastric ulcer, representatives of the gastrointestinal diseases, are defined as the conditions where gastrointestinal mucus membrane is digested by gastric acid to form ulcer. In the stomach walls generally consisting of mucosa, submucosa, muscle layer and serosa, gastric ulcer even damages submucosa and muscle layer, while gastritis damages mucosa only. Although the morbidity rates of gastritis and gastric ulcer are relatively high, the causes thereof have not been clarified yet. Until now, they are known to be caused by an imbalance between aggressive factors and defensive factors, that is, the increase in aggressive factors such as the increase in gastric acid or pepsin secretion, or the decrease in defensive factors such as structural or morphological deficit of the gastric mucus membrane, the decrease in mucus and bicarbonate ion secretion, the decrease in prostaglandin production, or the like.

Currently available therapeutic agents for gastritis and gastric ulcer comprise various drugs for strengthening the defensive factors such as an antacid, which does not affect, gastric acid secretion but neutralizes gastric acid that has been already produced, an inhibitor of gastric acid secretion, a promoter of prostaglandin secretion, and a coating agent for stomach walls. Especially, prostaglandins are known to be essential in maintaining the mechanism for protecting and defending gastric mucus membrane (Wallace J L., 2008, Physiol Rev., 88(4), 1547-65, S. J. Konturek et al., 2005, Journal of Physiology and Pharmacology, 56(5)). In view of the above, since the 15-PGDH inhibitors described herein show a suppressive or inhibitory activity against 15-PGDH, which degrades prostaglandins that protect gastric mucus membrane, they can be effective for the prevention or the treatment of gastrointestinal diseases, inter alia, gastritis and gastric ulcer.

Additionally, corticosteroids and TNF alpha antagonists are both used in the treatment of ulcerative colitis and IBD patients. In mouse models, 15-PGDH inhibitors speed healing of ulcerative colitis. We have found that administering corticosteroids to mice elevates levels of colon 15-PGDH, an effect that should reduce the therapeutic effectiveness of corticosteroids in colitis treatment. This suggests that combining a corticosteroid with a 15-PGDH inhibitor should be more effective in colitis (and IBD) treatment than using either agent alone.

Similarly, we have shown that TNF-alpha suppresses colon 15-PGDH expression. This suggests that TNF-alpha antagonists will increase colon 15-PGDH expression, an effect that should reduce the therapeutic effectiveness of corticosteroids in colitis treatment. This suggests that combining a TNF-alpha antagonist, e.g., the chimeric antibody REMICADE (infliximab), with a 15-PGDH inhibitor should be more effective in colitis (and IBD) treatment than using either agent alone.

In other embodiments, the 15-PGDH inhibitors and corticosteroids or 15-PGDH inhibitors and TNF inhibitors can be provided in a topical composition or formulation that is used to treat inflammation and/or aberrant immune system activity associated with medical conditions, such as atopic dermatitis, psoriasis, eczematous dermatitis, nummular dermatitis, irritant contact dermatitis, allergic contact dermatitis (such as poison ivy exposure, poison oak exposure, and poison sumac exposure), seborrheic dermatitis, stasis dermatitis, and other steroid responsive dermatoses.

In other embodiments, the 15-PGDH inhibitors and corticosteroids or 15-PGDH inhibitors and TNF inhibitors provided in a topical composition can be used to treat, for example, acne vulgaris, alopecia, alopecia greata, vitiligo, eczema, xerotic eczema, keratosis pilaris, Lichen planus, Lichen sclerosus, Lichen striatus, Lichen simplex chronicus, prurigo nodularis, discoid lupus erythematosus, lymphocytic infiltrate of Jessner/Kanof, lymphacytoma cutis, pyoderma gangrenosum, pruritis ani, sarcoidosis, chondrodermatitis nodularis helices, and other inflammatory dermatological disorders.

Medical conditions treated by the 15-PGDH inhibitors and corticosteroids or 15-PGDH inhibitors and TNF inhibitors can also include, for example, keloids, hypertrophic scars, pretibial myxedema and other infiltrative dermatological disorders. Additional medical conditions include, for example, granuloma annulare, necrobiosis lipoidica diabeticorum, sarcoidosis, and other noninfectious granulomas.

In still other embodiments, the 15-PGDH inhibitors described herein can be administered in combination with corticosteroids or TNF inhibitors for wound healing, tissue regeneration, and/or tissue repair. Among various prostaglandins, PGE₂ is known to serve as a mediator for wound healing. Therefore, subjects who are receiving steroids, including those healing of wounds from undergoing surgery, can be administered a 15-PGDH inhibitor to enhance PGE₂ and promote would healing.

Additionally, increased prostaglandin levels have been shown to stimulate signaling through the Wnt signaling pathway via increased beta-catenin mediated transcriptional activity. Wnt signaling is known to be a key pathway employed by tissue stem cells. Hence, 15-PGDH inhibitors described herein may be utilized to increase tissue stem cell numbers for purposes that would include promoting tissue regeneration or repair in subjects receiving corticosteroid treatment. In addition, 15-PGDH inhibitors described herein may be utilized to promote tissue regeneration or repair in additional organs that would include but are not limited to brain, eye, cornea, retina, lung, heart, stomach, small intestine, pancreas, beta-cells of the pancreas, kidney, bone, cartilage, and peripheral nerve.

In other embodiments, the 15-PGDH inhibitor can be used as a glucocorticoid sensitizer to treat glucocorticoid insensitivity, restore corticosteroid sensitivity, enhance glucocorticoid sensitivity, and/or reverse the glucocorticoid insensitivity in a subject experiencing corticosteroid dependence or corticoid resistance or unresponsiveness or intolerance to corticosteroids. Therapeutic effects of the 15-PGDH inhibitors when used as a glucocorticoid sensitizer include any, but are not limited to, steroid-sparing in corticosteroid-dependent patients, better responsiveness or tolerance to corticosteroids, achieving efficacy by using a lower dose of corticosteroid, preventing individuals at risk for developing refractory responses or resistance or exacerbations in response to antigen exposures, infections, exercise, or irritants, achieving optimal immune functions, easier responses for the subject or patient when steroid administration is tapered or withdrawn, or after prolonged administration of corticosteroids, decreased risks for developing corticosteroid-related adverse events such as opportunistic infections, bone loss, pathologic fracture, diabetes, cataract, and combinations thereof.

In some embodiments, the 15-PGDH inhibitor can be administered to a subject in combination with the corticosteroid to treat glucocorticoid insensitivity, restore corticosteroid sensitivity, enhance glucocorticoid sensitivity, and/or reverse the glucocorticoid insensitivity in a subject experiencing corticosteroid dependence or corticoid resistance or unresponsiveness or intolerance to corticosteroids. The glucocorticoid insensitivity related conditions can include a range of immune-inflammatory disorders/diseases treated with steroids when the therapy fails to achieve disease control or is not effective or intolerant or dependent to corticosteroids, and combinations thereof.

In other embodiments, the 15-PGDH inhibitor and corticosteroid or the 15-PGDH inhibitor and TNF inhibitor can be administered to a subject that exhibits one or more glucocorticoid insensitivity related diseases, disorders, or conditions selected from the group consisting of glucocorticoid resistant asthma, refractory rheumatoid arthritis, refractory inflammatory bowel disease, chronic obstructive pulmonary disease, acute respiratory distress syndrome, interstitial pulmonary fibrosis, cystic fibrosis, refractory ulcerative colitis, children with severe Crohn's disease, corticosteroid refractory asthma, desquamative interstitial pneumonia refractory to corticosteroid, refractory inflammatory myopathies, refractory myasthenia gravis, refractory pemphigus vulgaris, methotrexate-refractory RA patients, refractory nephrotic syndrome, refractory multiple sclerosis, refractory sprue-like disease, steroid-resistant sarcoidosis, refractory mucosal lesions of pemphigus vulgaris, refractory Schnitzler syndrome, resistant dermatitis of the head and neck, severe refractory atopic dermatitis, refractory Idiopathic thrombocytopenia purpura, refractory orbital myositis, refractory or recurrent lymphomas, critically ill patients with sepsis or acute respiratory distress syndrome (ARDS) and relative adrenal insufficiency, rosacea, polymyalgia rheumatic, giant cell arteritis, polymyositis, dermatomyositis, Kawasaki syndrome, Guillain-Barre syndrome, chronic inflammatory demyelinating polyneuropathy, multifocal motor neuropathy, Stiff man syndrome, corticosteroid dependent systemic lupus erythematosus, corticosteroid dependent multiple sclerosis, symptomatic corticosteroid dependent asthma, primary Sjogren's syndrome, systemic vasculitis, polymyositis, organ transplants, graft-versus-host disease, inflammatory diseases, autoimmune diseases, hyperproliferative diseases, lupus, osteoarthritis, rhinosinusitis, polyarteritis nodosa, Wegener's granulomatosis, giant cell arteritis, allergic rhinitis, urticaria, hereditary angioedema, tendonitis, bursitis, autoimmune chronic active hepatitis, cirrhosis, transplant rejection, psoriasis, dermatitus, malignancies, leukemia, myelomas, lymphomas, acute adrenal insufficiency, rheumatic fever, granulomatous disease, immune proliferation/apotosis, hypothalamic-pituitary-adrenal (HPA) axis suppression and regulation, hypercortisolemia, modulation of the Th1/Th2 cytokine balance, chronic kidney disease, spinal cord injury, cerebral edema, thrombocytopenia, Little's syndrome, Addison's disease, autoimmune hemolytic anemia, uveitis, pemphigus vulgaris, nasal polyps, sepsis, bacterial infections, viral infections, rickettsial infections, parasitic infections, type II diabetes, obesity, metabolic syndrome, depression, schizophrenia, mood disorders, Cushing's syndrome, anxiety, sleep disorders, memory and learning enhancement, glucocorticoid-induced glaucoma, atopic dermatitis, drug hypersensitivity reactions, serum sickness, bullous dermatitis herpetiformis, contact dermatitis, exfoliative erythroderma, mycosis fungoides, pemphigus, nonsuppurative thyroiditis, sympathetic ophthalmia, uveitis, ocular inflammatory conditions unresponsive to topical steroids, allergic bronchopulmonary aspergillosis, fulminating or disseminated pulmonary tuberculosis when used concurrently with appropriate chemotherapy, hypersensitivity pneumonitis, idiopathic bronchiolitis obliterans with organizing pneumonia, idiopathic eosinophilic pneumonias, idiopathic pulmonary fibrosis, Pneumocystis carinii pneumonia (PCP) associated with hypoxemia occurring in an HIV(+) individual who is also under treatment with appropriate anti-PCP antibiotics, a diuresis or remission of proteinuria in nephrotic syndrome, without uremia, of the idiopathic type or that due to lupus erythematosus, ankylosing spondylitis, polymyalgia rheumatic, psoriatic arthritis, relapsing polychondritis, trichinosis with neurologic or myocardial involvement, and tuberculous meningitis.

It will be appreciated that other 15-PGDH inhibitors can be used in the methods described described herein. These other 15-PGDH inhibitors can include known 15-PGDH inhibitors including, for example, tetrazole compounds of formulas (I) and (II), 2-alkylideneaminooxyacetamidecompounds of formula (I), heterocyclic compounds of fourmulas (VI) and (VII), and pyrazole compounds of formula (III) described in U.S. Patent Application Publication No. 2006/0034786 and U.S. Pat. No. 7,705,041; benzylidene-1,3-thiazolidine compounds of formula (I) described in U.S. Patent Application Publication No. 2007/0071699; phenylfurylmethylthiazolidine-2,4-dione and phenylthienylmethylthiazolidine-2,4-dione compounds described in U.S. Patent Application Publication No. 2007/0078175; thiazolidenedione derivatives described in U.S. Patent Application Publication No. 2011/0269954; phenylfuran, phenylthiophene, or phenylpyrrazole compounds described in U.S. Pat. No. 7,294,641; 5-(3,5-disubstituted phenylazo)-2-hydroxybenzene-acetic acids and salts; and lactones described in U.S. Pat. No. 4,725,676; azo compounds described in U.S. Pat. No. 4,889,846; and 15-PGHD inhibitors described in PCT/US2014/060761 and US Patent Application Publication No. 2015/0072998A1, all of which are herein incorporated by reference in their entirety.

The 15-PGDH inhibitors described herein can be provided in a pharmaceutical composition or cosmetic composition depending on the pathological or cosmetic condition or disorder being treated. A pharmaceutical composition containing the 15-PGDH inhibitors described herein as an active ingredient may be manufactured by mixing the derivative with a pharmaceutically acceptable carrier(s) or an excipient(s) or diluting the 15-PGDH inhibitors with a diluent in accordance with conventional methods. The pharmaceutical composition may further contain fillers, anti-cohesives, lubricants, wetting agents, flavoring agents, emulsifying agents, preservatives and the like. The pharmaceutical composition may be formulated into a suitable formulation in accordance with the methods known to those skilled in the art so that it can provide an immediate, controlled or sustained release of the 15-PGDH inhibitors after being administered into a mammal.

In some embodiments, the pharmaceutical composition may be formulated into a parenteral or oral dosage form. The solid dosage form for oral administration may be manufactured by adding excipient, if necessary, together with binder, disintegrants, lubricants, coloring agents, and/or flavoring agents, to the 15-PGDH inhibitors and shaping the resulting mixture into the form of tablets, sugar-coated pills, granules, powder or capsules. The additives that can be added in the composition may be ordinary ones in the art. For example, examples of the excipient include lactose, sucrose, sodium chloride, glucose, starch, calcium carbonate, kaolin, microcrystalline cellulose, silicate and the like. Exemplary binders include water, ethanol, propanol, sweet syrup, sucrose solution, starch solution, gelatin solution, carboxymethylcellulose, hydroxypropyl cellulose, hydroxypropyl starch, methylcellulose, ethylcellulose, shellac, calcium phosphonate and polypyrrolidone. Examples of the disintegrant include dry starch, sodium arginate, agar powder, sodium bicarbonate, calcium carbonate, sodium lauryl sulfate, stearic monoglyceride and lactose. Further, purified talc, stearates, sodium borate, and polyethylene glycol may be used as a lubricant; and sucrose, bitter orange peel, citric acid, tartaric acid, may be used as a flavoring agent. In some embodiments, the pharmaceutical composition can be made into aerosol formulations (e.g., they can be nebulized) to be administered via inhalation.

The 15-PGDH inhibitors described herein may be combined with flavoring agents, buffers, stabilizing agents, and the like and incorporated into oral liquid dosage forms such as solutions, syrups or elixirs in accordance with conventional methods. One example of the buffers may be sodium citrate. Examples of the stabilizing agents include tragacanth, acacia and gelatin.

In some embodiments, the 15-PGDH inhibitors described herein may be incorporated into an injection dosage form, for example, for a subcutaneous, intramuscular or intravenous route by adding thereto pH adjusters, buffers, stabilizing agents, relaxants, topical anesthetics. Examples of the pH adjusters and the buffers include sodium citrate, sodium acetate and sodium phosphate. Examples of the stabilizing agents include sodium pyrosulfite, EDTA, thioglycolic acid and thiolactic acid. The topical anesthetics may be procaine HCl, lidocaine HCl and the like. The relaxants may be sodium chloride, glucose and the like.

In other embodiments, the 15-PGDH inhibitors described herein may be incorporated into suppositories in accordance with conventional methods by adding thereto pharmaceutically acceptable carriers that are known in the art, for example, polyethylene glycol, lanolin, cacao butter or fatty acid triglycerides, if necessary, together with surfactants such as Tween.

The pharmaceutical composition may be formulated into various dosage forms as discussed above and then administered through various routes including an oral, inhalational, transdermal, subcutaneous, intravenous or intramuscular route. The dosage can be a pharmaceutically effective amount. The pharmaceutically effective amount can be an amount of the 15-PGDH inhibitor to treat or improve alopecia, cardiovascular disease, gastrointestinal disease, wounds, and renal disease. The pharmaceutically effective amount of the compound will be appropriately determined depending on the kind and the severity of the disease to be treated, age, sex, body weight and the physical condition of the patients to be treated, administration route, duration of therapy and the like. Generally, the effective amount of the compound may be in the range of about 1 to 1,000 mg in the oral administration, about 0.1 to 500 mg in the intravenous administration, about 5 to 1,000 mg in the rectal administration. Generally, the daily dosage for adults is in the range of about 0.1 to 5,000 mg, preferably about to 1,000 mg but cannot be determined uniformly because it depends on age, sex, body weight and the physical condition of the patients to be treated. The formulation may be administered once a day or several times a day with a divided dose.

Cosmetic compositions containing the 15-PGDH inhibitor can include any substance or preparation intended to be brought into contact with the various superficial parts of the human body (epidermis, body hair and hair system, nails, lips and external genital organs) or with the teeth or the buccal mucous membranes for the purpose, exclusively or mainly, of cleansing them, of giving them a fragrance, of modifying their appearance and/or of correcting body odors and/or protecting them or of maintaining them in good condition.

The cosmetic composition can comprise a cosmetically acceptable medium that may be water or a mixture of water and at least one solvent selected from among hydrophilic organic solvents, lipophilic organic solvents, amphiphilic organic solvents, and mixtures thereof.

For topical application, the cosmetic composition can be administered in the form of aqueous, alcoholic, aqueous-alcoholic or oily solutions or suspensions, or of a dispersion of the lotion or serum type, of emulsions that have a liquid or semi-liquid consistency or are pasty, obtained by dispersion of a fatty phase in an aqueous phase (O/W) or vice versa (W/O) or multiple emulsions, of a free or compacted powder to be used as it is or to be incorporated into a physiologically acceptable medium, or else of microcapsules or microparticles, or of vesicular dispersions of ionic and/or nonionic type. It may thus be in the form of a salve, a tincture, milks, a cream, an ointment, a powder, a patch, an impregnated pad, a solution, an emulsion or a vesicular dispersion, a lotion, aqueous or anhydrous gels, a spray, a suspension, a shampoo, an aerosol or a foam. It may be anhydrous or aqueous. It may also comprise solid preparations constituting soaps or cleansing cakes.

The cosmetic compositions may in particular comprise a hair care composition, and in particular a shampoo, a setting lotion, a treating lotion, a styling cream or gel, restructuring lotions for the hair, a mask, etc. The cosmetic compositions can be a cream, a hair lotion, a shampoo or a conditioner. These can be used in particular in treatments using an application that may or may not be followed by rinsing, or else in the form of a shampoo. A composition in the form of a foam, or else in the form of spray or an aerosol, then comprising propellant under pressure, is also intended. It can thus be in the form of a lotion, serum, milk, cream, gel, salve, ointment, powder, balm, patch, impregnated pad, cake or foam.

In particular, the compositions for application to the scalp or the hair can be in the form of a hair care lotion, for example for daily or twice-weekly application, of a shampoo or of a hair conditioner, in particular for twice-weekly or weekly application, of a liquid or solid soap for cleansing the scalp, for daily application, of a hairstyle shaping product (lacquer, hair setting product or styling gel), of a treatment mask, or of a foaming gel or cream for cleansing the hair. These may also be in the form of a hair dye or mascara to be applied with a brush or a comb.

Moreover, for topical application to the eyelashes or body hair, the compositions may be in the form of a pigmented or unpigmented mascara, to be applied with a brush to the eyelashes or alternatively to beard or moustache hair. For a composition administration by injection, the composition may be in the form of an aqueous lotion or an oily suspension. For oral use, the composition may be in the form of capsules, granules, oral syrups or tablets. According to a particular embodiment, the composition is in the form of a hair cream or hair lotion, a shampoo, a hair conditioner or a mascara for the hair or for the eyelashes.

In a known manner, the cosmetic compositions may also contain adjuvants that are normal in the cosmetics field, such as hydrophilic or lipophilic gelling agents, hydrophilic or lipophilic additives, preservatives, antioxidants, solvents, fragrances, fillers, UV-screening agents, odor absorbers and dyestuffs. The amounts of these various adjuvants are those conventionally used in the cosmetics field, and are for example from 0.1% to 20%, in particular less than or equal to 10%, of the total weight of the composition. According to their nature, these adjuvants can be introduced into the fatty phase, into the aqueous phase and/or into the lipid spherules.

In some embodiments, the 15-PGDH inhibitor can be administered in a combinatorial therapy or combination therapy that includes administration of a 15-PGDH inhibitor with one or more additional active agents. The phrase “combinatorial therapy” or “combination therapy” embraces the administration of the 15-PGDH inhibitor, and one or more therapeutic agents as part of a specific treatment regimen intended to provide beneficial effect from the co-action of these therapeutic agents. Administration of these therapeutic agents in combination typically is carried out over a defined period (usually minutes, hours, days or weeks depending upon the combination selected). “Combinatorial therapy” or “combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example by administering to the subject an individual dose having a fixed ratio of each therapeutic agent or in multiple, individual doses for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissue. The therapeutic agents can be administered by the same route or by different routes. The sequence in which the therapeutic agents are administered is not narrowly critical.

In some embodiments, the additional active agent can be chosen in particular from lipoxygenase inhibitors as described in EP 648488, the bradykinin inhibitors described in particular in EP 845700, prostaglandins and their derivatives, in particular those described in WO 98/33497, WO 95/11003, JP 97-100091, JP 96-134242, the agonists or antagonists of the receptors for prostaglandins, and the nonprostanoic analogues of prostaglandins as described in EP 1175891 and EP 1175890, WO 01/74307, WO 01/74313, WO 01/74314, WO 01/74315 or WO 01/72268.

In other embodiments, the 15-PGDH inhibitors can be administered in combination with active agents, such as vasodilators, prostanoid agonists, antiandrogens, cyclosporins and their analogues, antimicrobials, triterpenes, alone or as a mixture. The vasodilators can include potassium channel agonists including minoxidil and its derivatives, aminexil and the compounds described in U.S. Pat. Nos. 3,382,247, 5,756,092, 5,772,990, 5,760,043, 5,466,694, 5,438,058, 4,973,474, chromakalin and diazoxide. The antiandrogens can include 5.alpha.-reductase inhibitors such as finasteride and the compounds described in U.S. Pat. No. 5,516,779, cyprosterone acetate, azelaic acid, its salts and its derivatives, and the compounds described in U.S. Pat. No. 5,480,913, flutamide and the compounds described in U.S. Pat. Nos. 5,411,981, 5,565,467 and 4,910,226. The antimicrobial compounds can include selenium derivatives, ketoconazole, triclocarban, triclosan, zinc pyrithione, itraconazole, pyridine acid, hinokitiol, mipirocine, and the compounds described in EP 680745, clinycine hydrochloride, benzoyl or benzyl peroxide and minocycline. The anti-inflammatory agents can include inhibitors specific for Cox-2 such as for example NS-398 and DuP-697 (B. Batistini et al., DN&P 1994; 7(8):501-511) and/or inhibitors of lipoxygenases, in particular 5-lipoxygenase, such as for example zileuton (F. J. Alvarez & R. T. Slade, Pharmaceutical Res. 1992; 9(11):1465-1473).

Other active compounds, which can be present in pharmaceutical and/or cosmetic compositions can include aminexil and its derivatives, 60-[(9Z,12Z)octadec-9,12-dienoyl]hexapyranose, benzalkonium chloride, benzethonium chloride, phenol, oestradiol, chlorpheniramine maleate, chlorophyllin derivatives, cholesterol, cysteine, methionine, benzyl nicotinate, menthol, peppermint oil, calcium panthotenate, panthenol, resorcinol, protein kinase C inhibitors, prostaglandin H synthase 1 or COX-1 activators, or COX-2 activators, glycosidase inhibitors, glycosaminoglycanase inhibitors, pyroglutamic acid esters, hexosaccharidic or acylhexosaccharidic acids, substituted ethylenearyls, N-acylated amino acids, flavonoids, derivatives and analogues of ascomycin, histamine antagonists, triterpenes, such as ursolic acid and the compounds described in U.S. Pat. Nos. 5,529,769, 5,468,888, 5,631,282, saponins, proteoglycanase inhibitors, agonists and antagonists of oestrogens, pseudopterins, cytokines and growth factor promoters, IL-1 or IL-6 inhibitors, IL-10 promoters, TNF inhibitors, vitamins, such as vitamin D, analogues of vitamin B12 and panthotenol, hydroxy acids, benzophenones, esterified fatty acids, and hydantoin.

Pharmaceutical and/or cosmetic compositions including the 15-PGDH inhibitor described herein can additionally contain, for example, at least one compound chosen from prostaglandins, in particular prostaglandin PGE₁, PGE₂, their salts, their esters, their analogues and their derivatives, in particular those described in WO 98/33497, WO 95/11003, JP 97-100091, JP 96-134242, in particular agonists of the prostaglandin receptors. It may in particular contain at least one compound such as the agonists (in acid form or in the form of a precursor, in particular in ester form) of the prostaglandin F_{2α} receptor, such as for example latanoprost, fluprostenol, cloprostenol, bimatoprost, unoprostone, the agonists (and their precursors, in particular the esters such as travoprost) of the prostaglandin E₂ receptors such as 17-phenyl PGE₂, viprostol, butaprost, misoprostol, sulprostone, 16,16-dimethyl PGE₂, 11-deoxy PGE₁, 1-deoxy PGE₁, the agonists and their precursors, in particular esters, of the prostacycline (IP) receptor such as cicaprost, iloprost, isocarbacycline, beraprost, eprostenol, treprostinil, the agonists and their precursors, in particular the esters, of the prostaglandin D₂ receptor such as BW245C ((4S)-(3-[(3R,S)-3-cyclohexyl-3-isopropyl]-2,5-dioxo)-4-imidazolidinehept-anoic acid), BW246C ((4R)-(3-[(3R,S)-3-cyclohexyl-3-isopropyl]-2,5-dioxo)-4-imidazolidinehept-anoic acid), the agonists and their precursors, in particular the esters, of the receptor for the thromboxanes A2 (TP) such as I-BOP ([1S-[1a,2a(Z), 3b(1E,3S),4a]]-7-[3-[3-hydroxy-4-[4-(iodophenoxy)-1-butenyl]-7-oxabicyclo-[2.2.1]hept-2-yl]-5-heptenoic acid).

Advantageously, the composition can include at least one 15-PGDH inhibitor as defined above and at least one prostaglandin or one prostaglandin derivative such as for example the prostaglandins of series 2 including in particular PGF_{2α} and PGE₂ in saline form or in the form of precursors, in particular of the esters (example isopropyl esters), their derivatives such as 16,16-dimethyl PGE₂, 17-phenyl PGE₂ and 16,16-dimethyl PGF_{2α} 17-phenyl PGF_{2α}, prostaglandins of series 1 such as 11-deoxyprostaglandin E1, 1-deoxyprostaglandin E1 in saline or ester form, is their analogues, in particular latanoprost, travoprost, fluprostenol, unoprostone, bimatoprost, cloprostenol, viprostol, butaprost, misoprostol, their salts or their esters.

The invention is further illustrated by the following examples, which is not intended to limit the scope of the claims.

### Example 1

### Analysis of Analogues of Lead Compounds SW033291, a 15-PGDH Inhibitor

This Example provides data on a group of structural analogues of SW033291. Data provided is the IC₅₀ of each compound for inhibiting enzymatic activity of recombinant 15-PGDH in an in vitro assay. Recombinant 15-PGDH is human unless otherwise specified. Additionally, the example provides aqueous solubility data for selected analogues in pH 7 or pH 4 citrate buffer solution.

TABLE 1

| | | | Enzyme Inhibitor | Enzyme Inhibitor |
|---|---|---|---|---|
| | | Optical | IC₅₀ (nM) at 5 nM | IC₅₀ (nM) at 1 nM |
| Identifier | Structure/Smiles | Isomer | 15-PGDH | 15-PGDH |
| SW209415 | 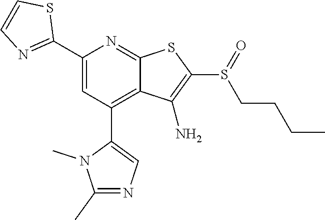 [SMILES: CCCCS(=O)c1sc2nc(-c3nccs3)cc(-c3cnc(C)n3C)c2c1N] | | 2.1 | |
| | CCCCS(═O)C1═C(N)C2═C(C═C(N═C2S1)C1═NC═CS1) | | | |
| | C1═CN═C(C)N1C | | | |
| 1 | 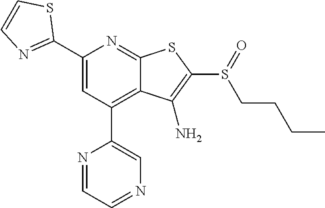 [SMILES: CCCCS(=O)c1sc2nc(-c3nccs3)cc(-c3cnccn3)c2c1N] | | 2.7 | |
| | CCCCS(═O)C1═C(N)C2═C(C═C(N═C2S1)C1═NC═CS1) | | | |
| | C1═CN═CC═N1 | | | |
| 2 | 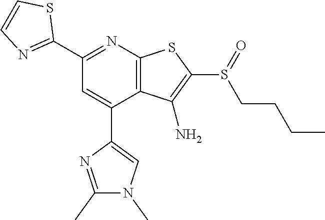 [SMILES: CCCCS(=O)c1sc2nc(-c3nccs3)cc(-c3cn(C)c(C)n3)c2c1N] | | 2.3 | |
| | CCCCS(═O)C1═C(N)C2═C(S1)N═C(C═C2C1═CN | | | |
| | (C)C(C)═N1)C1═NC═CS1 | | | |
| 3 | 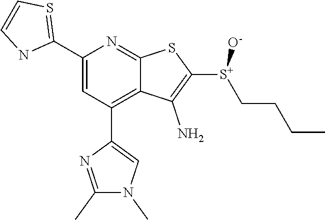 | (−) | >500 | |
| | CCCC[S@+]([O—]) | | | |
| | C1═C(N)C2═C(C═C(N═C2S1)C1═NC═CS1) | | | |
| | C1═CN(C)C(C)═N1 | | | |
| 4 | 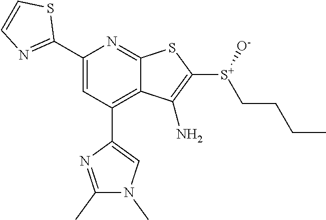 [SMILES: CCCC[S@@+]([O-])c1sc2nc(-c3nccs3)cc(-c3cn(C)c(C)n3)c2c1N] | (+) | 1.4 | |
| | CCCC[S@@+]([O—]) | | | |
| | C1═C(N)C2═C(C═C(N═C2S1)C1═NC═CS1) | | | |
| | C1═CN(C)C(C)═N1 | | | |
| 5 | 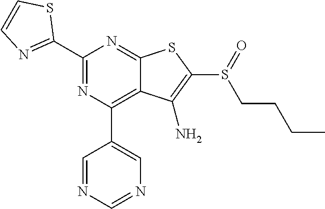 [SMILES: CCCCS(=O)c1sc2nc(-c3nccs3)nc(-c3cncnc3)c2c1N] | | not tested | |
| | CCCCS(C1═C(C2═C(C3═CN═CN═C3)N═C(C4═NC═CS4) | | | |
| | N═C2S1)N)═O | | | |
| 6 | 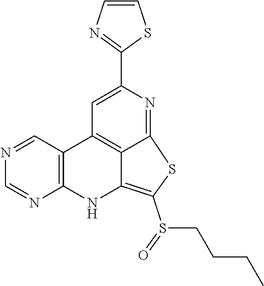 [SMILES: CCCCS(=O)c1sc2nc(-c3nccs3)cc3c2c1Nc1ncncc1-3] | | 86 | |
| | CCCCS(═O)C1═C2NC3═C(C═NC═N3)C3═C2C | | | |
| | (S1)═NC(═C3)C1═NC═CS1 | | | |
| 7 | 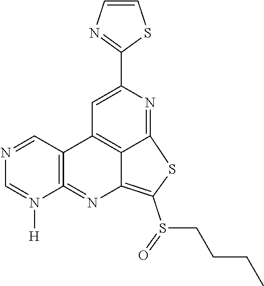 | | 132 | |
| | [H]N1C═NC═C2C3═C4C(SC(S(CCCC)═O)═C4N═Cl2)═NC | | | |
| | (C5═NC═CS5)═C3 | | | |
| 8 | 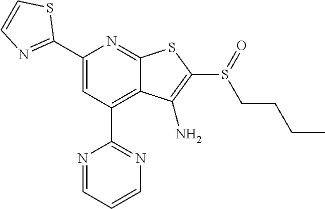 [SMILES: CCCCS(=O)c1sc2nc(-c3nccs3)cc(-c3ncccn3)c2c1N] | | 2.6 | |
| | CCCCS(═O)C1═C(N)C2═C(C═C(N═C2S1)C1═NC═CS1) | | | |
| | C1═NC═CC═N1 | | | |
| 10 | 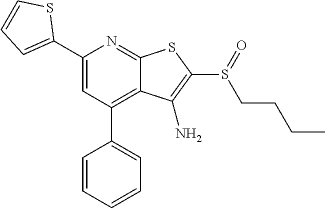 [SMILES: CCCCS(=O)c1sc2nc(-c3cccs3)cc(-c3ccccc3)c2c1N] | | 7.1 | |
| | CCCCS(═O)C1═C(C)C2═C(C═C(N═C2S1)C1═CC═CS1) | | | |
| | C1═CC═CC═C1 | | | |
| 11 | 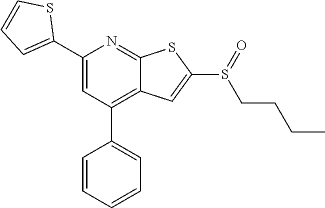 [SMILES: CCCCS(=O)c1cc2c(-c3ccccc3)cc(-c3cccs3)nc2s1] | | 24.7 | |
| | CCCCS(═O)C1═CC2═C(C═C(N═C2S1)C1═CC═CS1) | | | |
| | C1═CC═CC═C1 | | | |
| 12 | 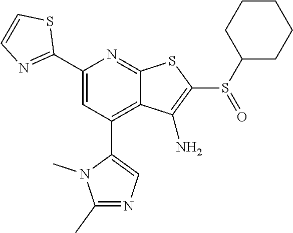 [SMILES: Cc1ncc(-c2cc(-c3nccs3)nc3sc(S(=O)C4CCCCC4)c(N)c23)n1C] | | 2.6 | |
| | O═S(C1═C(C2═C(C3═CN═C(N3C)C)C═C(C4═NC═CS4) | | | |
| | N═C2S1)N)C5CCCCC5 | | | |
| 13 | 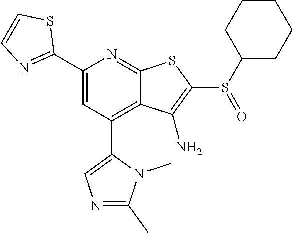 [SMILES: Cc1ncc(-c2cc(-c3nccs3)nc3sc(S(=O)C4CCCCC4)c(N)c23)n1C] | (+) | 0.76 | |
| | CN1C(C)═NC═C1C1═C2C(N)═C(SC2═NC(═C1) | | | |
| | C1═NC═CS1)S(═O)C1CCCCC1 | | | |
| 14 | 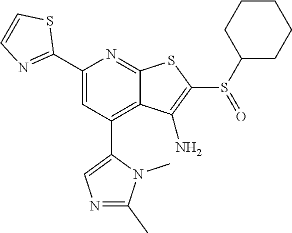 [SMILES: Cc1ncc(-c2cc(-c3nccs3)nc3sc(S(=O)C4CCCCC4)c(N)c23)n1C] | (−) | 186 | |
| | CN1C(C)═NC═C1C1═C2C(N)═C(SC2═NC(═C1) | | | |
| | C1═NC═CS1)S(═O)C1CCCCC1 | | | |
| 15 | 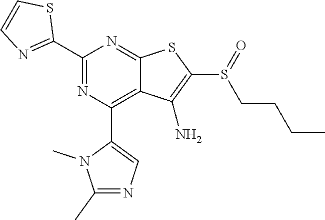 [SMILES: CCCCS(=O)c1sc2nc(-c3nccs3)nc(-c3cnc(C)n3C)c2c1N] | | 3.7 | |
| | CCCCS(═O)C1═C(N)C2═C(N═C(N═C2S1)C1═NC═CS1) | | | |
| | C1═CN═C(C)N1C | | | |
| 16 | 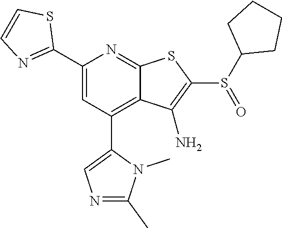 [SMILES: Cc1ncc(-c2cc(-c3nccs3)nc3sc(S(=O)C4CCCC4)c(N)c23)n1C] | | 3.3 | |
| | CN1C(C)═NC═C1C1═C2C(N)═C(SC2═NC(═C1) | | | |
| | C1═NC═CS1)S(═O)C1CCCC1 | | | |
| 17 | 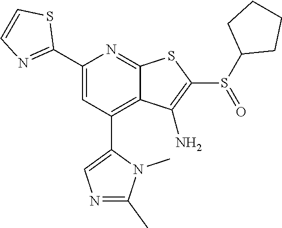 [SMILES: Cc1ncc(-c2cc(-c3nccs3)nc3sc(S(=O)C4CCCC4)c(N)c23)n1C] | (+) | 0.4 | |
| | CN1C(C)═NC═C1C1═C2C(N)═C(SC2═NC(═C1) | | | |
| | C1═NC═CS1)S(═O)C1CCCC1 | | | |
| 18 | 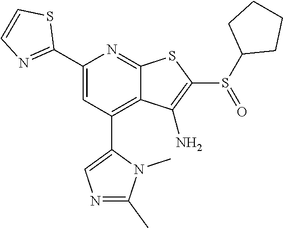 [SMILES: Cc1ncc(-c2cc(-c3nccs3)nc3sc(S(=O)C4CCCC4)c(N)c23)n1C] | (−) | 125 | |
| | CN1C(C)═NC═C1C1═C2C(N)═C(SC2═NC(═C1) | | | |
| | C1═NC═CS1)S(═O)C1CCCC1 | | | |
| 19 | 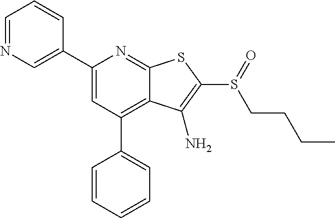 [SMILES: CCCCS(=O)c1sc2nc(-c3cccnc3)cc(-c3ccccc3)c2c1N] | | 2.4 | |
| | CCCCS(═O)C1═C(N)C2═C(C═C(N═C2S1)C1═CN═CC═C1) | | | |
| | C1═CC═CC═C1 | | | |
| 20 | 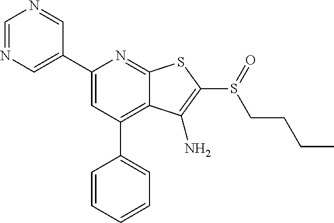 [SMILES: CCCCS(=O)c1sc2nc(-c3cncnc3)cc(-c3ccccc3)c2c1N] | | 3.2 | |
| | CCCCS(═O)C1═C(N)C2═C(C═C(N═C2S1)C1═CN═CN═C1) | | | |
| | C1═CC═CC═C1 | | | |
| 21 | 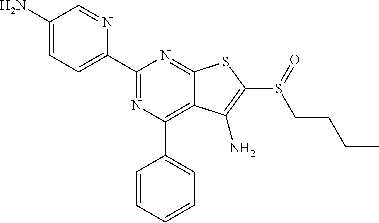 [SMILES: CCCCS(=O)c1sc2nc(-c3ccc(N)cn3)nc(-c3ccccc3)c2c1N] | | ~4 | |
| | CCCCS(═O)C1═C(N)C2═C(C═C(N═C2S1)C1═CN═C(N)N═Cl) | | | |
| | C1═CC═CC═C1 | | | |
| 22 | 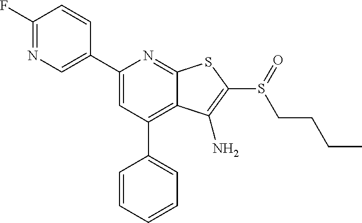 [SMILES: CCCCS(=O)c1sc2nc(-c3ccc(F)nc3)cc(-c3ccccc3)c2c1N] | | ~4 | |
| | CCCCS(═O)C1═C(N)C2═C(C═C(N═C2S1)C1═CN═C(F)C═C1) | | | |
| | C1═CC═CC═C1 | | | |
| 23 | 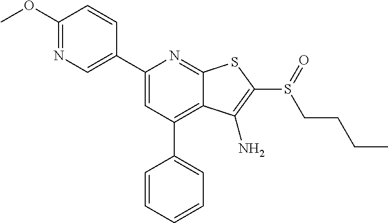 [SMILES: CCCCS(=O)c1sc2nc(-c3ccc(OC)nc3)cc(-c3ccccc3)c2c1N] | | 4-20 | |
| | CCCCS(═O)C1═C(N)C2═C(C═C(N═C2S1)C1═CN═C(OC)C═C1) | | | |
| | C1═CC═CC═C1 | | | |
| 24 | 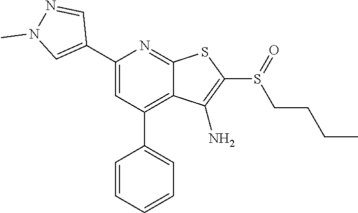 [SMILES: CCCCS(=O)c1sc2nc(-c3cnn(C)c3)cc(-c3ccccc3)c2c1N] | | 3 | |
| | CCCCS(═O)C1═C(N)C2═C(C═C(N═C2S1)C1═CN(C)N═C1) | | | |
| | C1═CC═CC═C1 | | | |
| 25 | 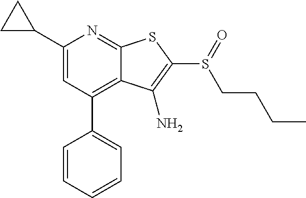 [SMILES: CCCCS(=O)c1sc2nc(C3CC3)cc(-c3ccccc3)c2c1N] | | 4-20 | |
| | CCCCS(═O)C1═C(N)C2═C(C═C(N═C2S1)C1 | | | |
| | CC1)C1═CC═CC═C1 | | | |
| 26 | 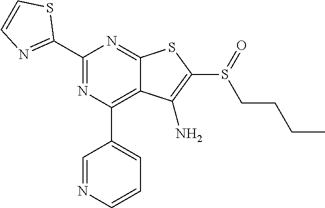 [SMILES: CCCCS(=O)c1sc2nc(-c3nccs3)nc(-c3cccnc3)c2c1N] | | ~20 | |
| | CCCCS(═O)C1═C(N)C2═C(N═C(N═C2S1)C1═NC═CS1) | | | |
| | C1═CC═CN═C1 | | | |
| 27 | 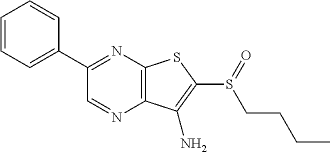 [SMILES: CCCCS(=O)c1sc2nc(-c3ccccc3)cnc2c1N] | | 1.6 | |
| | CCCCS(═O)C1═C(N)C2═C(S1)N═C(C═N2)C1═CC═CC═C1 | | | |
| 28 | 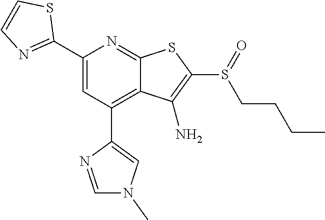 [SMILES: CCCCS(=O)c1sc2nc(-c3nccs3)cc(-c3cn(C)cn3)c2c1N] | | 1.4 | |
| | CCCCS(═O)C1═C(N)C2═C(C═C(N═C2S1)C1═NC═CS1) | | | |
| | C1═CN(C)C═N1 | | | |
| 29 | 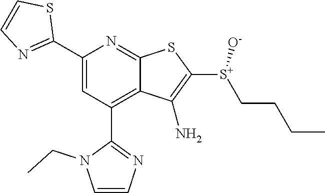 [SMILES: CCCC[S@@+]([O-])c1sc2nc(-c3nccs3)cc(-c3nccn3CC)c2c1N] | (+) | 1.4 | |
| | CCCC[S@@+]([O—1) | | | |
| | C1═C(N)C2═C(C═C(N═C2S1)C1═NC═CS1) | | | |
| | C1═NC═CN1CC | | | |
| 30 | 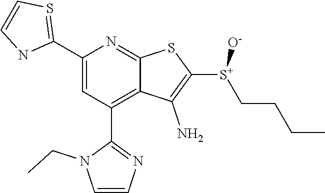 | (−) | 221 | |
| | CCCC[S@+([O—]) | | | |
| | C1═C(N)C2═C(C═C(N═C2S1)C1═NC═CS1) | | | |
| | C1═NC═CN1CC | | | |
| 31 | 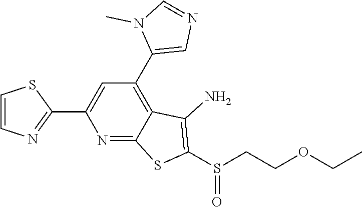 [SMILES: CCOCCS(=O)c1sc2nc(-c3nccs3)cc(-c3cncn3C)c2c1N] | | | 1.8 |
| | CCOCCS(═O)C1═C(N)C2═C(C═C(N═C2S1)C1═NC═CS1) | | | |
| | C1═CN═CN1C | | | |
| 31A | 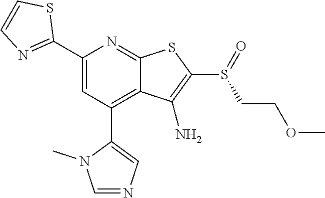 [SMILES: COCC[S@@](=O)c1sc2nc(-c3nccs3)cc(-c3cncn3C)c2c1N] | | | Not Tested |
| | COCC[S@@](═O)C1═C(N)C2═C(C═C(N═C2S1) | | | |
| | C1═NC═CS1)C1═CN═CN1C | | | |
| 32 | 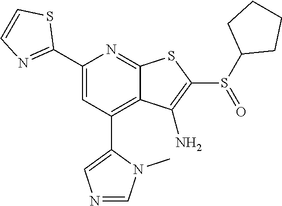 [SMILES: Cn1cncc1-c1cc(-c2nccs2)nc2sc(S(=O)C3CCCC3)c(N)c12] | | | 1.6 |
| | CN1C═NC═C1C1═C2C(N)═C(SC2═NC(═C1) | | | |
| | C1═NC_50 CS1)S(═O)C1CCCC1 | | | |
| 32A | 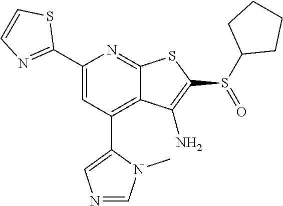 [SMILES: Cn1cncc1-c1cc(-c2nccs2)nc2sc([S@](=O)C3CCCC3)c(N)c12] | | | Not Tested |
| | CN1C═NC═C1C1═C2C(N)═C(SC2═NC(═C1) | | | |
| | C1═NC═CS1)[S@](═O)C1CCCC1 | | | |
| 33 | 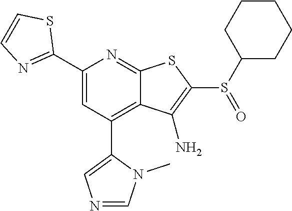 [SMILES: Cn1cncc1-c1cc(-c2nccs2)nc2sc(S(=O)C3CCCCC3)c(N)c12] | | | 1.6 |
| | CN1C═NC═C1C1═C2C(N)═C(SC2═NC(═C1) | | | |
| | C1═NC═CS1)S(═O)C1CCCCC1 | | | |
| 33A | 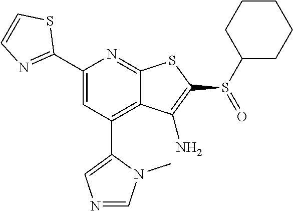 [SMILES: Cn1cncc1-c1cc(-c2nccs2)nc2sc([S@](=O)C3CCCCC3)c(N)c12] | | | Not Tested |
| | CN1C═NC═C1C1═C2(N)═C(SC2═NC(═C1) | | | |
| | C1═NC═CS1)[S@](═O)C1CCCCC1 | | | |
| 34 | 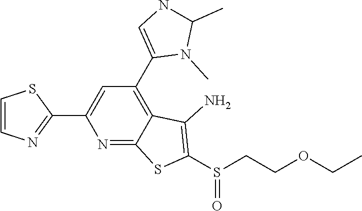 [SMILES: CCOCCS(=O)c1sc2nc(-c3nccs3)cc(C3=CNC(C)N3C)c2c1N] | | | 3.1 |
| | CCOCCS(═O)C1═C(N)C2═C(C═C(N═C2S1)C1═NC═CS1) | | | |
| | C1═CN═C(C)N1C | | | |
| 34A | 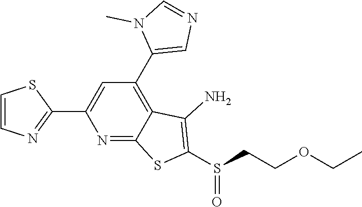 [SMILES: CCOCC[S@@](=O)c1sc2nc(-c3nccs3)cc(-c3cncn3C)c2c1N] | | | Not Tested |
| | CCOCC[S@@](═O)C1═C(N)C2═C(C═C(N═C2S1) | | | |
| | C1═NC═CS1)C1═CN═CN1C | | | |
| 35 | 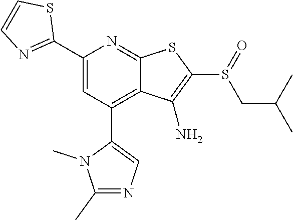 [SMILES: Cc1ncc(-c2cc(-c3nccs3)nc3sc(S(=O)CC(C)C)c(N)c23)n1C] | | | 3.6 |
| | CC(C)CS(═O)C1═C(N)C2═C(C═C(N═C2S1)C1═NC═CS1)C1═CN═C(C)N1C | | | |
| 36 | 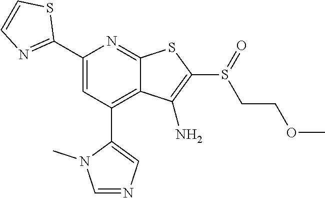 [SMILES: COCCS(=O)c1sc2nc(-c3nccs3)cc(-c3cncn3C)c2c1N] | | | 1.5 |
| | COCCS(═O)C1═C(N)C2═C(C═C(N═C2S1)C1═NC═CS1) | | | |
| | C1═CN═CN1C | | | |
| 37 | 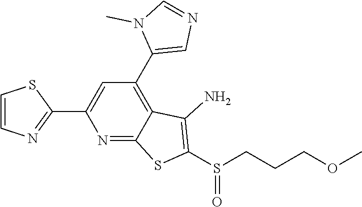 [SMILES: COCCCS(=O)c1sc2nc(-c3nccs3)cc(-c3cncn3C)c2c1N] | | | 1.8 |
| | COCCCS(═O)C1═C(N)C2═C(C═C(N═C2S1)C1═NC═CS1) | | | |
| | C1═CN═CN1C | | | |
| 37A | 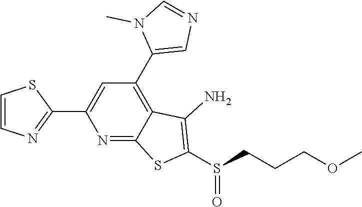 [SMILES: COCCC[S@@](=O)c1sc2nc(-c3nccs3)cc(-c3cncn3C)c2c1N] | | | Not tested |
| | COCCC[S@@](═O)C1═C(N)C2═C(C═C(N═C2S1) | | | |
| | C1═NC═CS1)C1═CN═CN1C | | | |
| 38 | 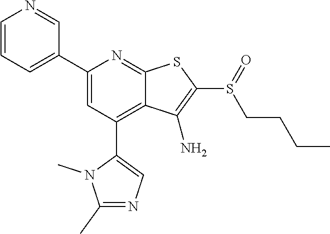 [SMILES: CCCCS(=O)c1sc2nc(-c3cccnc3)cc(-c3cnc(C)n3C)c2c1N] | | | 1.4 |
| | CCCCS(═O)C1═C(N)C2═C(C═C(N═C2S1)C1═CC═CN═C1) | | | |
| | C1═CN═C(C)N1C | | | |
| 38A | 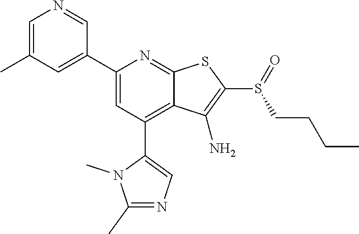 [SMILES: CCCC[S@@](=O)c1sc2nc(-c3cncc(C)c3)cc(-c3cnc(C)n3C)c2c1N] | (+) | | 0.76 |
| | CCCC[S@@](═O)C1═C(N)C2═C(C═C(N═C2S1) | | | |
| | C1═CC═CN═C1)C1═CN═C(C)N1C | | | |
| 38B | 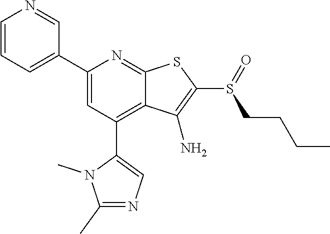 [SMILES: CCCC[S@](=O)c1sc2nc(-c3cccnc3)cc(-c3cnc(C)n3C)c2c1N] | (−) | | 27 |
| | CCCC[S@](═O)C1═C(N)C2═C(C═C(N═C2S1) | | | |
| | C1═CC═CN═C1)C1═CN═C(C)N1C | | | |
| 39 | 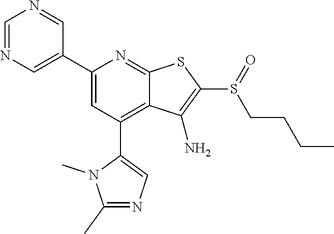 [SMILES: CCCCS(=O)c1sc2nc(-c3cncnc3)cc(-c3cnc(C)n3C)c2c1N] | | | 1.2 |
| | CCCCS(═O)C1═C(N)C2═C(C═C(N═C2S1)C1═CN═CN═C1) | | | |
| | C1═CN═C(C)N1C | | | |
| 39A | 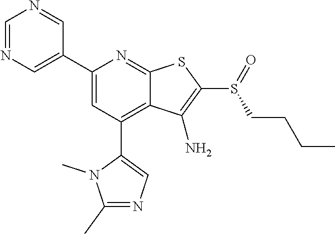 [SMILES: CCCC[S@@](=O)c1sc2nc(-c3cncnc3)cc(-c3cnc(C)n3C)c2c1N] | (+) | | 0.72 |
| | CCCC[S@@](═O)C1═C(N)C2═C(C═C(N═C2S1) | | | |
| | C1═CN═CN═C1)C1═CN═C(C)N1C | | | |
| 39B | 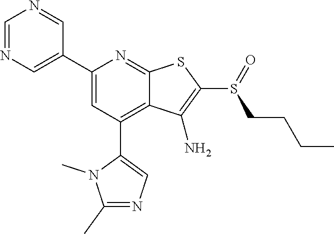 [SMILES: CCCC[S@](=O)c1sc2nc(-c3cncnc3)cc(-c3cnc(C)n3C)c2c1N] | (−) | | >100 |
| | CCCC[S@](═O)C1═C(N)C2═C(C═C(N═C2S1) | | | |
| | C1═CN═CN═C1)C1═CN═C(C)N1C | | | |
| 40 | 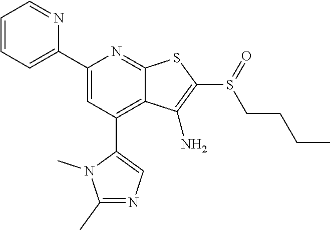 [SMILES: CCCCS(=O)c1sc2nc(-c3ccccn3)cc(-c3cnc(C)n3C)c2c1N] | | | 24 |
| | CCCCS(═O)C1═C(N)C2═C(C═C(N═C2S1)C1═CC═CC═N1) | | | |
| | C1═CN═C(C)N1C | | | |
| 41A | 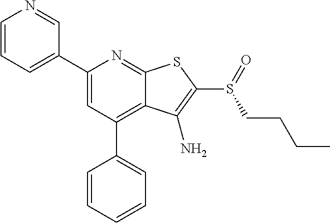 [SMILES: CCCC[S@@](=O)c1sc2nc(-c3cccnc3)cc(-c3ccccc3)c2c1N] | (+) | | 0.73 |
| | CCCC[S@@](═O)C1═C(N)C2═C(C═C(N═C2S1) | | | |
| | C1═CC═CN═C1)C1═CC═CC═C1 | | | |
| 41B | 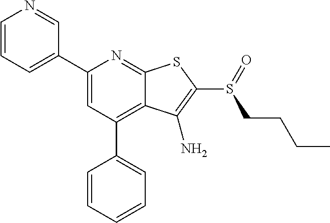 [SMILES: CCCC[S@](=O)c1sc2nc(-c3cccnc3)cc(-c3ccccc3)c2c1N] | (−) | | 27 |
| | CCCC[S@](═O)C1═C(N)C2═C(C═C(N═C2S1) | | | |
| | C1═CC═CN═C1)C1═CC═CC═C1 | | | |
| 42A | 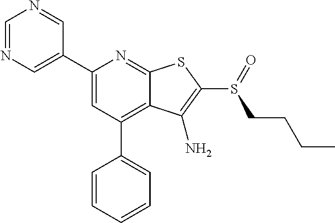 [SMILES: CCCC[S@](=O)c1sc2nc(-c3cncnc3)cc(-c3ccccc3)c2c1N] | (+) | | 30 |
| | CCCC[S@](═O)C1═C(N)C2═O(C═C(N═C2S1) | | | |
| | C1═CN═CN═C1)C1═CC═CC═C1 | | | |
| 42B | 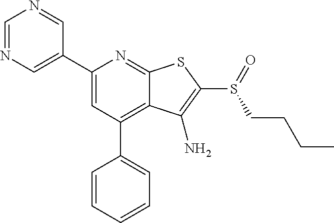 [SMILES: CCCC[S@@](=O)c1sc2nc(-c3cncnc3)cc(-c3ccccc3)c2c1N] | (−) | | 0.59 |
| | CCCC[S@@](═O)C1═C(N)C2═C(C═C(N═C2S1) | | | |
| | C1═CN═CN═C1)C1═CC═CC═C1 | | | |
| 43 | 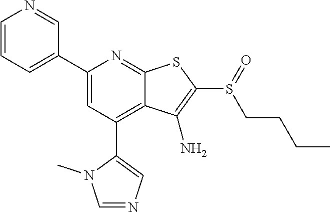 [SMILES: CCCCS(=O)c1sc2nc(-c3cccnc3)cc(-c3cncn3C)c2c1N] | | | 1.1 |
| | CCCCS(═O)C1═C(N)C2═C(C═C(N═C2S1)C1═CC═CN═C1) | | | |
| | C1═CN═CN1C | | | |
| 43A | 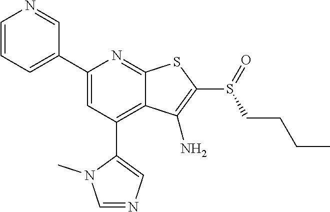 [SMILES: CCCC[S@@](=O)c1sc2nc(-c3cccnc3)cc(-c3cncn3C)c2c1N] | (+) | | 0.58 |
| | CCCC[S@@](═O)C1═C(N)C2═C(C═C(N═C2S1) | | | |
| | C1═CC═CN═C1)C1═CN═CN1C | | | |
| 43B | 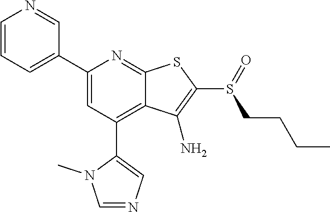 [SMILES: CCCC[S@](=O)c1sc2nc(-c3cccnc3)cc(-c3cncn3C)c2c1N] | (−) | | >100 |
| | CCCC[S@](═O)C1═C(N)C2═O(C═O(N═C2S1) | | | |
| | C1═CC═CN═C1)C1═CN═CN1C | | | |
| 44 | 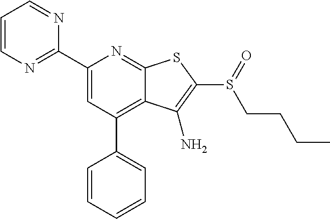 [SMILES: CCCCS(=O)c1sc2nc(-c3ncccn3)cc(-c3ccccc3)c2c1N] | | | 1.7 |
| | CCCCS(═O)C1═C(N)C2═C(C═C(N═C2S1)C1═NC═CC═N1) | | | |
| | C1═CC═CC═C1 | | | |
| 45 | 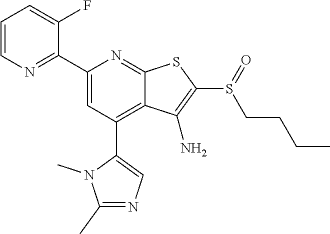 [SMILES: CCCCS(=O)c1sc2nc(-c3ncccc3F)cc(-c3cnc(C)n3C)c2c1N] | | | 22 |
| | CCCCS(═O)C1═C(N)C2═C(C═C(N═C2S1)C1═NC═CC═C1F) | | | |
| | C1═(CN═C(C)N1C | | | |
| 46A | 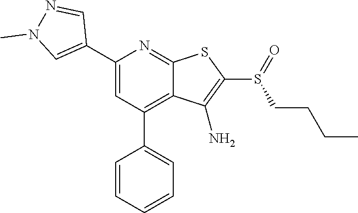 [SMILES: CCCC[S@@](=O)c1sc2nc(-c3cnn(C)c3)cc(-c3ccccc3)c2c1N] | (+) | | 0.7 |
| | CCCC[S@@](═O)C1═C(N)C2═C(C═C(N═C2S1) | | | |
| | C1═CN(C)N═C1)C1═CC═CC═C1 | | | |
| 46B | 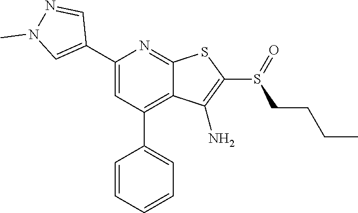 [SMILES: CCCC[S@](=O)c1sc2nc(-c3cnn(C)c3)cc(-c3ccccc3)c2c1N] | (−) | | >100 |
| | CCCC[S@](═O)C1═C(N)C2═C(C═C(N═C2S1) | | | |
| | C1═CN(C)N═C1)C1═CC═CC═C1 | | | |
| 47 | 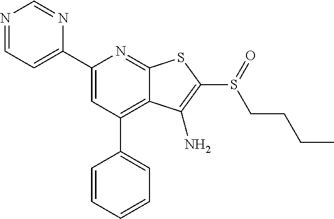 [SMILES: CCCCS(=O)c1sc2nc(-c3ccncn3)cc(-c3ccccc3)c2c1N] | | | 1.1 |
| | CCCCS(═O)C1═C(N)C2═C(C═C(N═C2S1)C1═CC═NC═N1) | | | |
| | C1═CC═CC═C1 | | | |
| 47A | 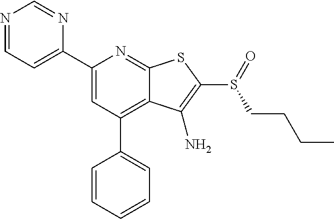 [SMILES: CCCC[S@@](=O)c1sc2nc(-c3ccncn3)cc(-c3ccccc3)c2c1N] | (+) | | 0.65 |
| | CCCC[S@@](═O)C1═C(N)C2═C(C═C(N═C2S1) | | | |
| | C1═CC═NC═N1)C1═CC═CC═C1 | | | |
| 47B | 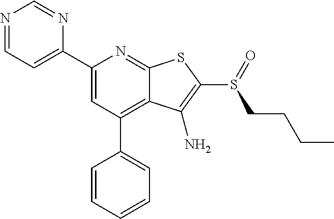 [SMILES: CCCC[S@](=O)c1sc2nc(-c3ccncn3)cc(-c3ccccc3)c2c1N] | (−) | | 79 |
| | CCCC[S@](═O)C1═C(N)C2═C(C═C(N═C2S1) | | | |
| | C1═CC═NC═N1)C1═CC═CC═C1 | | | |
| 48A | 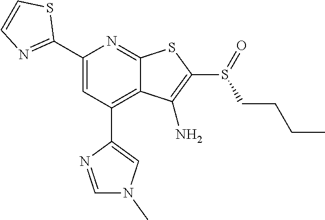 [SMILES: CCCC[S@@](=O)c1sc2nc(-c3nccs3)cc(-c3cn(C)cn3)c2c1N] | (+) | | 0.72 |
| | CCCC[S@@](═O)C1═C(N)C2═C(C═C(N═C2S1) | | | |
| | C1═NC═CS1)C1═CN(C)C═N1 | | | |
| 48B | 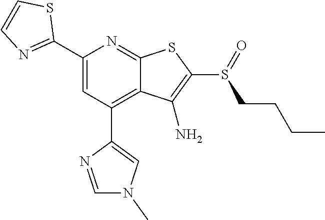 [SMILES: CCCC[S@](=O)c1sc2nc(-c3nccs3)cc(-c3cn(C)cn3)c2c1N] | (−) | | >100 |
| | CCCC[S@](═O)C1═C(N)C2═C(C═C(N═C2S1) | | | |
| | C1═NC═CS1)C1═CN(C)C═N1 | | | |
| 49 | 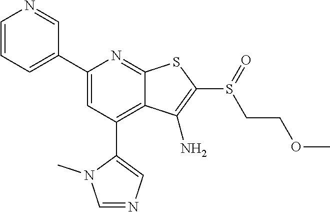 [SMILES: COCCS(=O)c1sc2nc(-c3cccnc3)cc(-c3cncn3C)c2c1N] | | | 1.8 |
| | COCCS(═O)C1═C(N)C2═C(C═C(N═C2S1)C1═CC═CN═C1) | | | |
| | C1═CN═CN1C | | | |
| 50 | 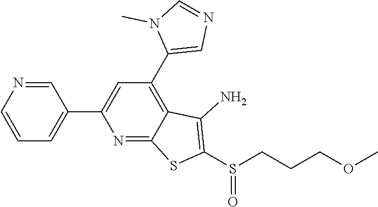 [SMILES: COCCCS(=O)c1sc2nc(-c3cccnc3)cc(-c3cncn3C)c2c1N] | | | 1.4 |
| | COCCCS(═O)C1═C(N)C2═C(C═C(N═C2S1)C1═CC═CN═C1) | | | |
| | C1═CN═CN1C | | | |
| 50A | 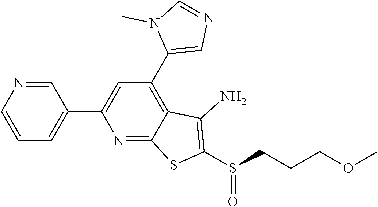 [SMILES: COCCC[S@@](=O)c1sc2nc(-c3cccnc3)cc(-c3cncn3C)c2c1N] | (+) | | 0.57 |
| | COCCC[S@@](═O)C1═C(N)C2═O(C═C(N═C2S1) | | | |
| | C1═CC═CN═C1)C1═CN═CN1C | | | |
| 50B | 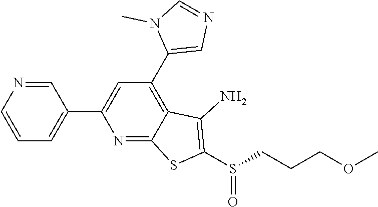 [SMILES: COCCC[S@](=O)c1sc2nc(-c3cccnc3)cc(-c3cncn3C)c2c1N] | (−) | | >100 |
| | COCCC[S@](═O)C1═C(N)C2═C(C═C(N═C2S1) | | | |
| | C1═CC═CN═C1)C1═CN═CN1C | | | |
| 51 | 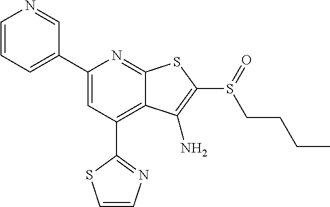 [SMILES: CCCCS(=O)c1sc2nc(-c3cccnc3)cc(-c3nccs3)c2c1N] | | | 1 |
| | CCCCS(═O)C1═C(N)C2═C(C═C(N═C2S1)C1═CC═CN═C1) | | | |
| | C1═NC═CS1 | | | |
| 52 | 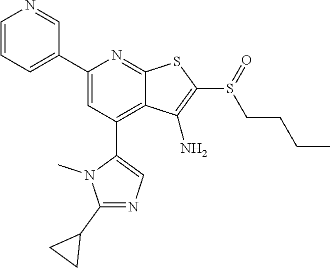 [SMILES: CCCCS(=O)c1sc2nc(-c3cccnc3)cc(-c3cnc(C4CC4)n3C)c2c1N] | | | 1.2 |
| | CCCCS(═O)C1═C(N)C2═C(C═C(N═C2S1)C1═CC═CN═C1) | | | |
| | C1═CN═C(C2CC2)N1C | | | |
| 52B | 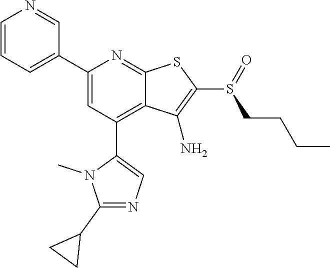 [SMILES: CCCC[S@](=O)c1sc2nc(-c3cccnc3)cc(-c3cnc(C4CC4)n3C)c2c1N] | (−) | | >100 |
| | CCCC[S@](═O)C1═C(N)C2═C(C═C(N═C2S1) | | | |
| | C1═CC═CN═C1)C1═CN═C(C2CC2)N1C | | | |
| 52A | 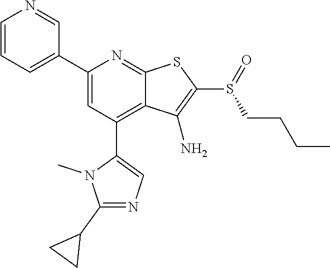 [SMILES: CCCC[S@@](=O)c1sc2nc(-c3cccnc3)cc(-c3cnc(C4CC4)n3C)c2c1N] | (+) | | 0.75 |
| | CCCC[S@@](═O)C1═C(N)C2═C(C═C(N═C2S1) | | | |
| | C1═CC═CN═C1)C1═CN═C(C2CC2)N1C | | | |
| 53 | 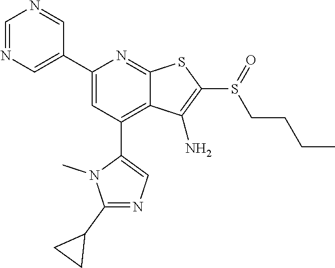 [SMILES: CCCCS(=O)c1sc2nc(-c3cncnc3)cc(-c3cnc(C4CC4)n3C)c2c1N] | | | 2.6 |
| | CCCCS(═O)C1═C(N)C2═C(C═C(N═C2S1)C1═CN═CN═C1) | | | |
| | C1═CN═C(C2CC2)N1C | | | |
| 54 | 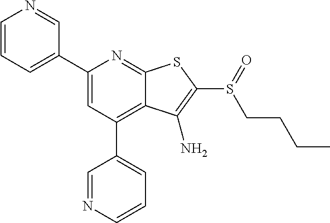 [SMILES: CCCCS(=O)c1sc2nc(-c3cccnc3)cc(-c3cccnc3)c2c1N] | | | 0.82 |
| | CCCCS(═O)C1═C(N)C2═C(C═C(N═C2S1)C1═CC═CN═C1) | | | |
| | C1═CC═CN═C1 | | | |
| 54A | 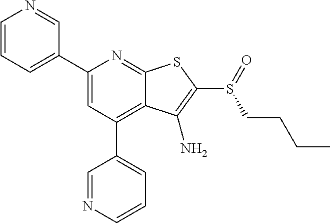 [SMILES: CCCC[S@@](=O)c1sc2nc(-c3cccnc3)cc(-c3cccnc3)c2c1N] | (+) | | 0.45 |
| | CCCC[S@@](═O)C1═C(N)C2═C(C═C(N═C2S1) | | | |
| | C1═CC═CN═C1)C1═CC═CN═C1 | | | |
| 54B | 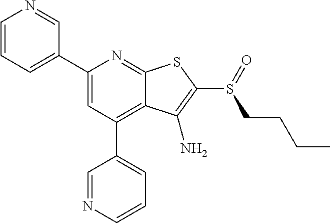 [SMILES: CCCC[S@](=O)c1sc2nc(-c3cccnc3)cc(-c3cccnc3)c2c1N] | (−) | | >100 |
| | CCCC[S@](═O)C1═C(N(C2═O(C═C(N═C2S1) | | | |
| | C1═CC═CN═C1)C1═CC═CN═C1 | | | |
| 55 | 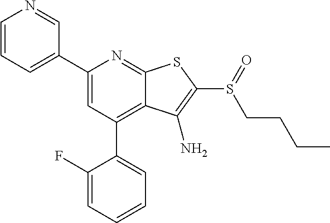 [SMILES: CCCCS(=O)c1sc2nc(-c3cccnc3)cc(-c3ccccc3F)c2c1N] | | | 1 |
| | CCCCS(═O)C1═C(N)C2═C(C═C(N═C2S1)C1═CC═CN═C1) | | | |
| | C1═CC═CC═C1F | | | |
| 56 | 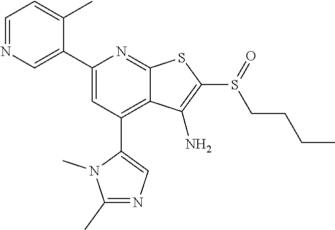 [SMILES: CCCCS(=O)c1sc2nc(-c3cnccc3C)cc(-c3cnc(C)n3C)c2c1N] | | | 2.2 |
| | CCCCS(═O)C1═C(N)C2═C(C═C(N═C2S1)C1═CN═CC═C1C) | | | |
| | C1═CN═C(C)N1C | | | |
| 57 | 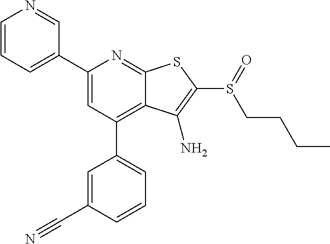 [SMILES: CCCCS(=O)c1sc2nc(-c3cccnc3)cc(-c3cccc(C#N)c3)c2c1N] | | | 1.1 |
| | CCCCS(═O)C1═C(N)C2═C(C═C(N═C2S1)C1═CC═CN═C1) | | | |
| | C1═CC═CC(═C1)C#N | | | |
| 58 | 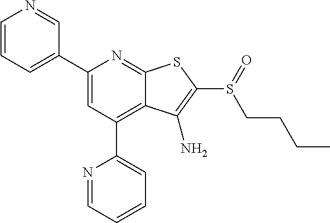 [SMILES: CCCCS(=O)c1sc2nc(-c3cccnc3)cc(-c3ccccn3)c2c1N] | | | 1.2 |
| | CCCCS(═O)C1═C(N)C2═C(C═C(N═C2S1)C1═CC═CN═C1) | | | |
| | C1═CC═CC═N1 | | | |
| 58A | 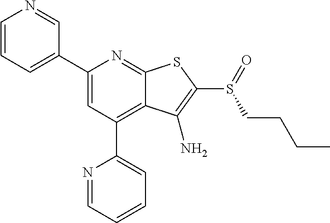 [SMILES: CCCC[S@@](=O)c1sc2nc(-c3cccnc3)cc(-c3ccccn3)c2c1N] | (+) | | 0.55 |
| | CCCC[S@@](═O)C1═C(N)C2═C(C═C(N═C2S1) | | | |
| | C1═CC═CN═C1)C1═CC═CC═N1 | | | |
| 58B | 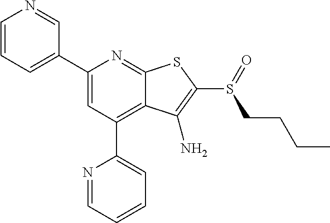 [SMILES: CCCC[S@](=O)c1sc2nc(-c3cccnc3)cc(-c3ccccn3)c2c1N] | (−) | | 0.13 |
| | CCCC[S@](═O)C1═C(N)C2═C(C═C(N═C2S1) | | | |
| | C1═CC═CN═C1)C1═CC═CC═N1 | | | |
| 59 | 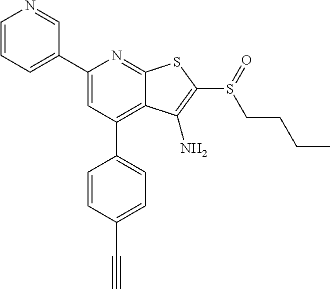 [SMILES: C#Cc1ccc(-c2cc(-c3cccnc3)nc3sc(S(=O)CCCC)c(N)c23)cc1] | | | 0.91 |
| | CCCCS(═O)C1═C(N)C2═C(C═C(N═C2S1)C1═CC═CN═C1) | | | |
| | C1═CC═C(C═C1)C#N | | | |
| 60 | 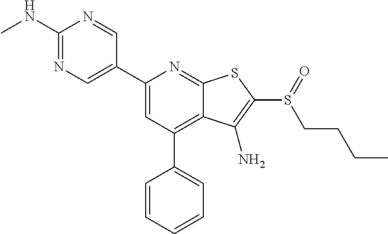 [SMILES: CCCCS(=O)c1sc2nc(-c3cnc(NC)nc3)cc(-c3ccccc3)c2c1N] | | | 1.4 |
| | CCCCS(═O)C1═C(N)C2═C(C═C(N═C2S1)C1═CN═C | | | |
| | (NC)N═C1)C1═CC═CC═C1 | | | |
| 60A | 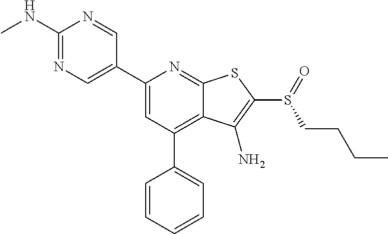 [SMILES: CCCC[S@@](=O)c1sc2nc(-c3cnc(NC)nc3)cc(-c3ccccc3)c2c1N] | (+) | | 0.5 |
| | CCCC[S@@](═O)C1═C(N)C2═C(C═C(N═C2S1) | | | |
| | C1═CN═C(NC)N═C1)C1═CC═CC═C1 | | | |
| 60B | 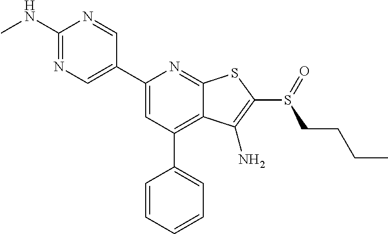 [SMILES: CCCC[S@](=O)c1sc2nc(-c3cnc(NC)nc3)cc(-c3ccccc3)c2c1N] | (−) | | 41 |
| | CCCC[S@](═O)C1═C(N)C2═C(C═C(N═C2S1) | | | |
| | C1═CN═C(NC)N═C1)C1═CC═CC═C1 | | | |
| 61 | 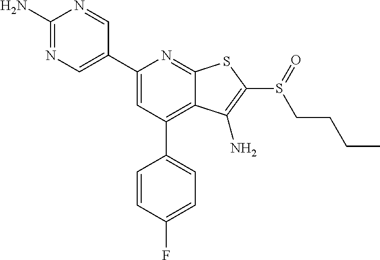 [SMILES: CCCCS(=O)c1sc2nc(-c3cnc(N)nc3)cc(-c3ccc(F)cc3)c2c1N] | | | 1.1 |
| | CCCCS(═O)C1═C(N)C2═C(C═C(N═C2S1)C1═CN═C | | | |
| | (N)N═C1)C1═CC═C(F)C═C1 | | | |
| 61A | 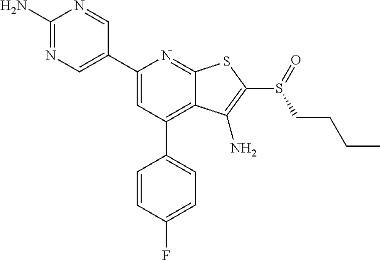 [SMILES: CCCC[S@@](=O)c1sc2nc(-c3cnc(N)nc3)cc(-c3ccc(F)cc3)c2c1N] | (+) | | 0.44 |
| | CCCC[S@@](═O)C1═C(N)C2═C(C═C(N═C2S1) | | | |
| | C1═CN═C(N,N═C1)C1═CC═C(F)C═C1 | | | |
| 61B | 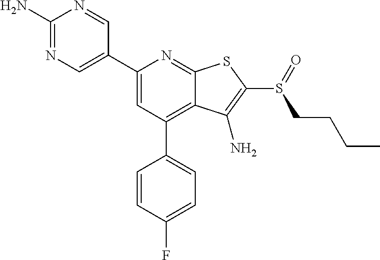 [SMILES: CCCC[S@](=O)c1sc2nc(-c3cnc(N)nc3)cc(-c3ccc(F)cc3)c2c1N] | (−) | | 24 |
| | CCCC[S@](═O)C1═C(N)C2═C(C═C(N═C2S1) | | | |
| | C1═CN═C(N)N═C1)C1═CC═C(F)C═C1 | | | |
| 62 | 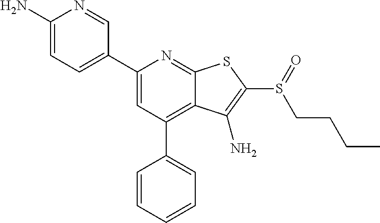 [SMILES: CCCCS(=O)c1sc2nc(-c3ccc(N)nc3)cc(-c3ccccc3)c2c1N] | | | 1.5 |
| | CCCCS(═O)C1═C(N)C2═C(C═C(N═C2S1)C1═CC═C | | | |
| | (N)N═C1)C1═CC═CC═C1 | | | |
| 62A | 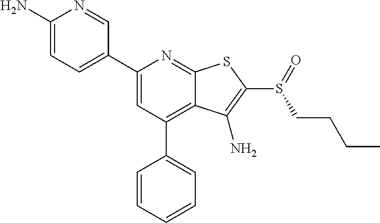 [SMILES: CCCC[S@@](=O)c1sc2nc(-c3ccc(N)nc3)cc(-c3ccccc3)c2c1N] | (+) | | 0.56 |
| | CCCC[S@@](═O)C1═C(N)C2═C(C═C(N═C2S1) | | | |
| | C1═CC═C(N)N═C1)C1═CC═CC═C1 | | | |
| 62B | 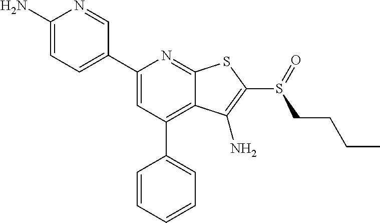 [SMILES: CCCC[S@](=O)c1sc2nc(-c3ccc(N)nc3)cc(-c3ccccc3)c2c1N] | (−) | | 64 |
| | CCCC[S@(═O)C1═C(N)C2═C(C═C(N═C2S1) | | | |
| | C1═CC═C(N)N═C1)C1═CC═CC═C1 | | | |
| 63 | 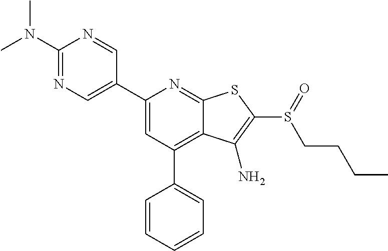 [SMILES: CCCCS(=O)c1sc2nc(-c3cnc(N(C)C)nc3)cc(-c3ccccc3)c2c1N] | | | 1.7 |
| | CCCS(═O)C1═C(N)C2═C(C═C(N═C2S1)C1═CN═C | | | |
| | (N═C1)N(C)C)C1═CC═CC═C1 | | | |
| 64A | 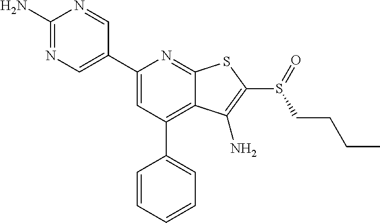 [SMILES: CCCC[S@@](=O)c1sc2nc(-c3cnc(N)nc3)cc(-c3ccccc3)c2c1N] | (+) | | 0.36 |
| | CCCC[S@@(═O)C1═C(N)C2═C(C═C(N═C2S1) | | | |
| | C1═CN═C(N)N═C1)C1═CC═CC═C1 | | | |
| 64B | 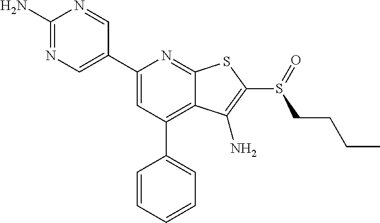 [SMILES: CCCC[S@](=O)c1sc2nc(-c3cnc(N)nc3)cc(-c3ccccc3)c2c1N] | (−) | | >100 |
| | CCCC[S@](═O)C1═C(N)C2═C(C═C(N═C2S1) | | | |
| | C1═CN═C(N)N═C1)C1═CC═CC═C1 | | | |
| 65 | 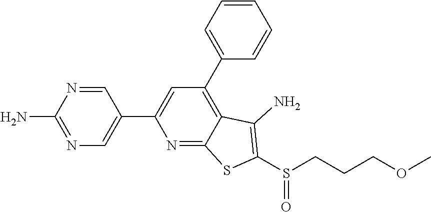 [SMILES: COCCCS(=O)c1sc2nc(-c3cnc(N)nc3)cc(-c3ccccc3)c2c1N] | | | 1.1 |
| | COCCCS(═O)C1═C(N)C2═C(C═C(N═C2S1)C1═CN═C | | | |
| | (N)N═C1)C1═CC═CC═C1 | | | |
| 65A | 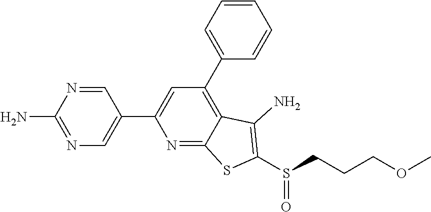 [SMILES: COCCC[S@@](=O)c1sc2nc(-c3cnc(N)nc3)cc(-c3ccccc3)c2c1N] | (+) | | 0.59 |
| | COCCC[S@@](═O)C1═C(N)C2═C(C═C(N═C2S1) | | | |
| | C1═CN═C(N)N═C1)C1═CC═CC═C1 | | | |
| 65B | 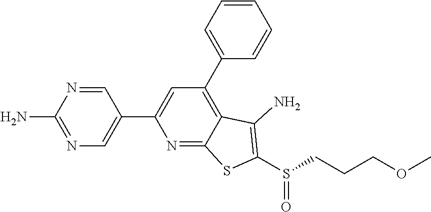 [SMILES: COCCC[S@](=O)c1sc2nc(-c3cnc(N)nc3)cc(-c3ccccc3)c2c1N] | (−) | | 30 |
| | COCCC[S@](═O)C1═C(N)C2═C(C═C(N═C2S1) | | | |
| | C1═CN═C(N)N═C1)C1═CC═CC═C1 | | | |
| 66 | 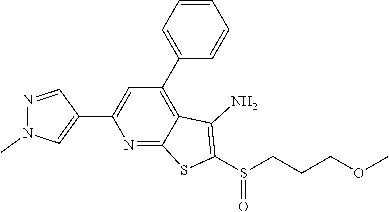 | | | 1.4 |
| | COCCCS(═O)C1═C(N)C2═C(C═C(N═C2S1)C1═CN | | | |
| | (C)N═C1)C1═CC═CC═C1 | | | |
| 67 | 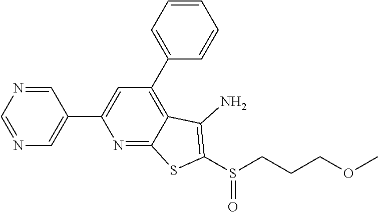 [SMILES: COCCCS(=O)c1sc2nc(-c3cncnc3)cc(-c3ccccc3)c2c1N] | | | 1.1 |
| | COCCCS(═O)C1═C(N)C2═C(C═C(N═C2S1)C1═CN═CN═C1) | | | |
| | C1═CC═CC═C1 | | | |
| 67A | 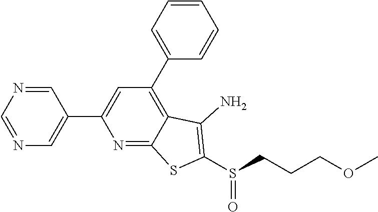 [SMILES: COCCC[S@@](=O)c1sc2nc(-c3cncnc3)cc(-c3ccccc3)c2c1N] | (+) | | 0.52 |
| | COCCC[S@@](═O)C1═C(N)C2═C(C═C(N═C2S1) | | | |
| | C1═CN═CN═C1)C1═CC═CC═C1 | | | |
| 67B | 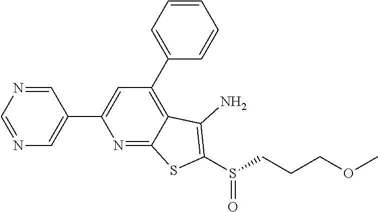 [SMILES: COCCC[S@](=O)c1sc2nc(-c3cncnc3)cc(-c3ccccc3)c2c1N] | (−) | | 40 |
| | COCCC[S@](═O)C1═C(N)C2═C(C═C(N═C2S1) | | | |
| | C1═CN═CN═C1)C1═CC═CC═C1 | | | |
| 68A | 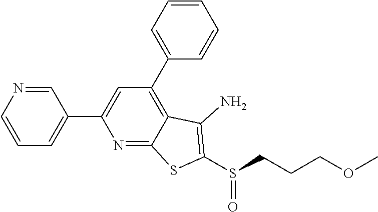 [SMILES: COCCC[S@@](=O)c1sc2nc(-c3cccnc3)cc(-c3ccccc3)c2c1N] | (+) | | 0.48 |
| | COCCC[S@@](═O)C1═C(N)C2═C(C═C(N═C2S1) | | | |
| | C1═CC═CN═C1)C1═CC═CC═C1 | | | |
| 68B | 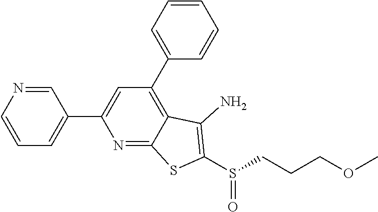 [SMILES: COCCC[S@](=O)c1sc2nc(-c3cccnc3)cc(-c3ccccc3)c2c1N] | (−) | | >100 |
| | COCCC[S@](═O)C1═C(N)C2═C(C═C(N═C2S1) | | | |
| | C1═CC═CN═C1)C1═CC═CC═C1 | | | |
| 69 | 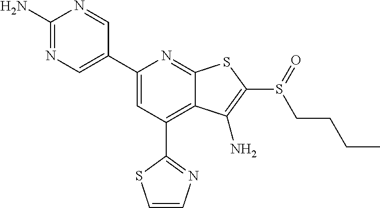 [SMILES: CCCCS(=O)c1sc2nc(-c3cnc(N)nc3)cc(-c3nccs3)c2c1N] | | | 1.1 |
| | CCCCS(═O)C1═C(N)C2═C(C═C(N═C2S1)C1═CN═C | | | |
| | (N)N═C1)C1═NC═CS1 | | | |
| 70 | 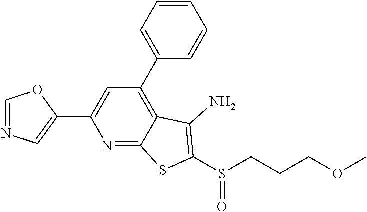 [SMILES: COCCCS(=O)c1sc2nc(-c3cnco3)cc(-c3ccccc3)c2c1N] | | | 0.88 |
| | COCCCS(═O)C1═C(N)C2═C(C═C(N═C2S1)C1═CN═CO1) | | | |
| | C1═CC═CC═C1 | | | |
| 71 | 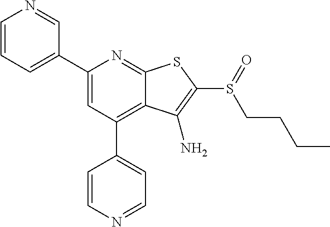 [SMILES: CCCCS(=O)c1sc2nc(-c3cccnc3)cc(-c3ccncc3)c2c1N] | | | 0.98 |
| | CCCCS(═O)C1═C(N)C2═C(C═C(N═C2S1)C1═CC═CN═C1) | | | |
| | C1═CC═NC═C1 | | | |
| 72 | 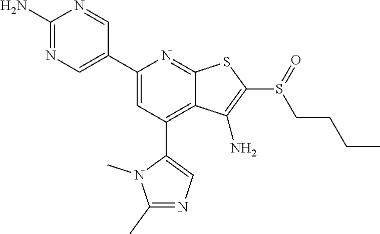 [SMILES: CCCCS(=O)c1sc2nc(-c3cnc(N)nc3)cc(-c3cnc(C)n3C)c2c1N] | | | 0.93 |
| | CCCCS(═O)C1═C(N)C2═C(C═C(N═C2S1)C1═CN═C | | | |
| | (N)N═C1)C1═CN═C(C)N1C | | | |
| 73 | 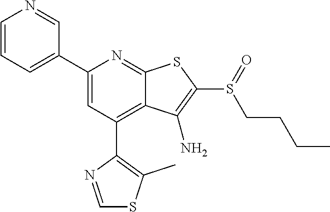 [SMILES: CCCCS(=O)c1sc2nc(-c3cccnc3)cc(-c3ncsc3C)c2c1N] | | | 1 |
| | CCCCS(═O)C1═C(N)C2═C(C═C(N═C2S1)C1═CC═CN═C1) | | | |
| | C1═C(C)SC═N1 | | | |
| 74 | 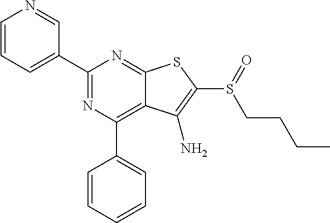 [SMILES: CCCCS(=O)c1sc2nc(-c3cccnc3)nc(-c3ccccc3)c2c1N] | | | 1.2 |
| | CCCCS(═O)C1═C(N)C2═C(N═C(N═C2S1)C1═CC═CN═C1) | | | |
| | C1═CC═CC═C1 | | | |
| 74B | 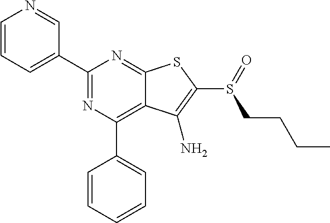 [SMILES: CCCC[S@](=O)c1sc2nc(-c3cccnc3)nc(-c3ccccc3)c2c1N] | (−) | | 28 |
| | CCCC[S@[(═O)C1═C(N)C2═C(N═C(N═C2S1) | | | |
| | C1═CC═CN═C1)C1═CC═CC═C1 | | | |
| 74A | 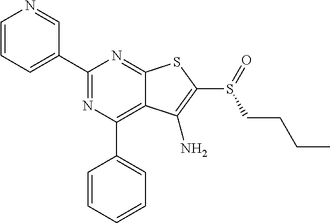 [SMILES: CCCC[S@@](=O)c1sc2nc(-c3cccnc3)nc(-c3ccccc3)c2c1N] | (+) | | 0.52 |
| | CCCC[S@@](═O)C1═C(N)C2═C(N═C(N═C2S1) | | | |
| | C1═CC═CN═C1)C1═CC═CC═C1 | | | |
| 75 | 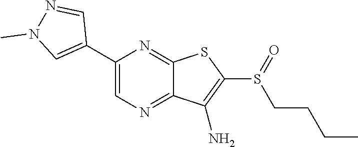 [SMILES: CCCCS(=O)c1sc2nc(-c3cnn(C)c3)cnc2c1N] | | | 2.8 |
| | CCCCS(═O)C1═C(N)C2═NC═C(N═C2S1)C1═CN(C)N═C1 | | | |
| 76 | 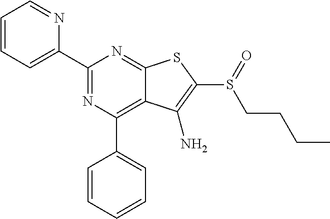 [SMILES: CCCCS(=O)c1sc2nc(-c3ccccn3)nc(-c3ccccc3)c2c1N] | | | 3.6 |
| | CCCCS(═O)C1═C(N)C2═C(N═C(N═C2S1)C1═CC═CC═N1) | | | |
| | C1═CC═CC═C1 | | | |
| 77 | 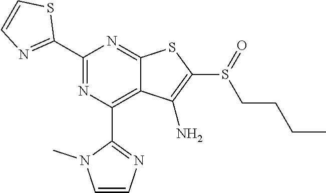 [SMILES: CCCCS(=O)c1sc2nc(-c3nccs3)nc(-c3nccn3C)c2c1N] | | | 1.8 |
| | CCCCS(═O)C1═C(N)C2═C(N═C(N═C2S1)C1═NC═CS1) | | | |
| | C1═NC═CN1C | | | |
| 78 | 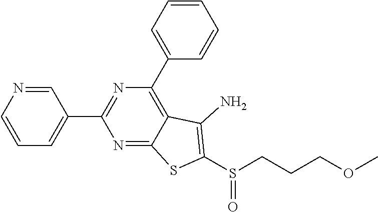 [SMILES: COCCCS(=O)c1sc2nc(-c3cccnc3)nc(-c3ccccc3)c2c1N] | | | 1.4 |
| | COCCCS(═O)C1═C(N)C2═C(N═C(N═C2S1) | | | |
| | C1═CC═CN═C1)C1═CC═CC═C1 | | | |
| 78-A | 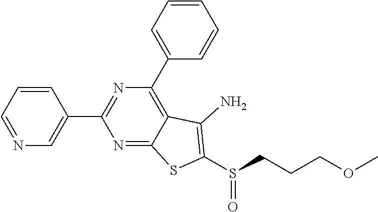 [SMILES: COCCC[S@@](=O)c1sc2nc(-c3cccnc3)nc(-c3ccccc3)c2c1N] | (+) | | 1.0 |
| | COCCC[S@](C1═C(C2═C(C3═CC═CC═C3)N═C | | | |
| | (C4═CN═CC═C4)N═C2S1)N)═O | | | |
| 78-B | 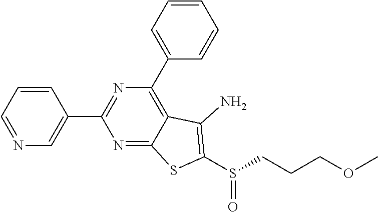 [SMILES: COCCC[S@](=O)c1sc2nc(-c3cccnc3)nc(-c3ccccc3)c2c1N] | (−) | | inactive |
| | COCCC[S@@](C1═C(C2═C(C3═CC═CC═C3) | | | |
| | N═C(C4═CN═CC═C4)N═C2S1)N)═O | | | |
| 79 | 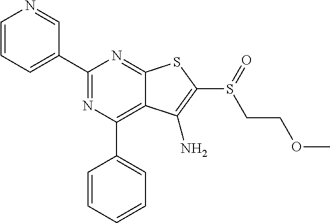 [SMILES: COCCS(=O)c1sc2nc(-c3cccnc3)nc(-c3ccccc3)c2c1N] | | | 1.5 |
| | COCCS(═O)C1═C(N)C2═C(N═C(N═C2S1)C1═CC═CN═C1) | | | |
| | C1═CC═CC═C1 | | | |
| 79-A | 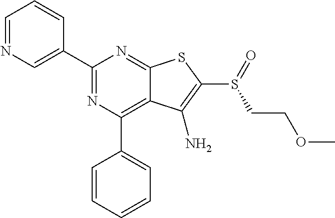 [SMILES: COCC[S@@](=O)c1sc2nc(-c3cccnc3)nc(-c3ccccc3)c2c1N] | (+) | | 1.0 |
| | COCC[S@](C1═C(C2═C(C3═CC═CC═C3)N═C | | | |
| | (C4═CN═CC═C4)N═C2S1)N)═O | | | |
| 79-B | 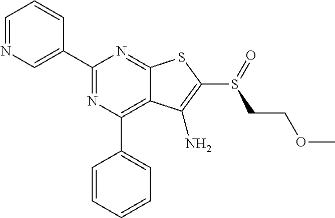 [SMILES: COCC[S@](=O)c1sc2nc(-c3cccnc3)nc(-c3ccccc3)c2c1N] | (−) | | inactive |
| | COCC[S@@](C1═C(C2═C(C3═CC═CC═C3) | | | |
| | N═C(C4═CN═CC═C4)N═C2S1)N)═O | | | |
| 80 | 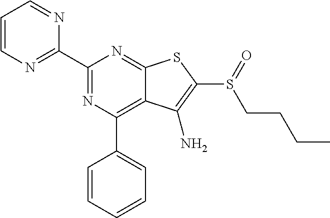 [SMILES: CCCCS(=O)c1sc2nc(-c3ncccn3)nc(-c3ccccc3)c2c1N] | | | 12 |
| | CCCS(═O)C1═C(N)C2═C(N═C(N═C2S1)C1═NC═CC═N1) | | | |
| | C1═CC═CC═C1 | | | |
| 81 | 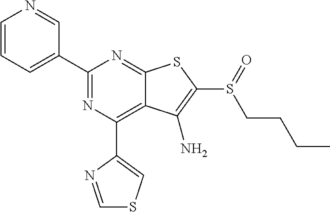 [SMILES: CCCCS(=O)c1sc2nc(-c3cccnc3)nc(-c3cscn3)c2c1N] | | | 0.76 |
| | CCCCS(═O)C1═C(N)C2═C(N═C(N═C2S1)C1═CC═CN═C1) | | | |
| | C1═CSC═N1 | | | |
| 82 | 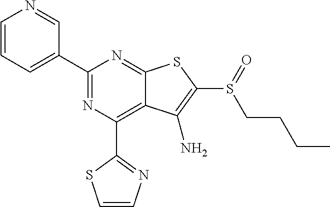 [SMILES: CCCCS(=O)c1sc2nc(-c3cccnc3)nc(-c3nccs3)c2c1N] | | | 1 |
| | CCCCS(═O)C1═C(N)C2═C(N═C(N═C2S1)C1═CC═CN═C1) | | | |
| | C1═NC═CS1 | | | |
| 83 | 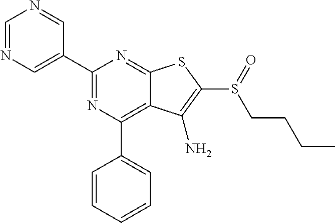 [SMILES: CCCCS(=O)c1sc2nc(-c3cncnc3)nc(-c3ccccc3)c2c1N] | | | 1.3 |
| | CCCCS(═O)C1═C(N)C2═C(N═C(N═C2S1)C1═CN═CN═C1) | | | |
| | C1═CC═CC═C1 | | | |
| 84 | 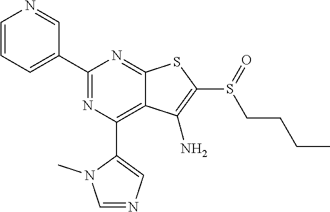 [SMILES: CCCCS(=O)c1sc2nc(-c3cccnc3)nc(-c3cncn3C)c2c1N] | | | 1.2 |
| | CCCCS(═O)C1═C(N)C2═C(N═C(N═C2S1)C1═CC═CN═C1) | | | |
| | C1═CN═CN1C | | | |
| 85 | 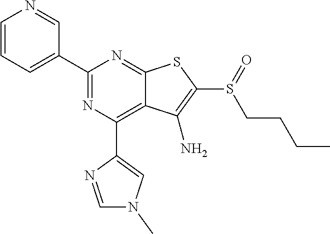 [SMILES: CCCCS(=O)c1sc2nc(-c3cccnc3)nc(-c3cn(C)cn3)c2c1N] | | | 1.3 |
| | CCCCS(═O)C1═C(N)C2═C(N═C(N═C2S1)C1═CC═CN═C1) | | | |
| | C1═CN(C)C═N] | | | |
| 86 | 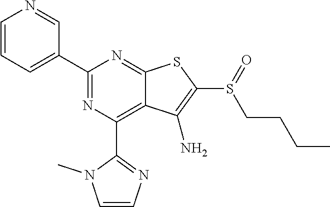 [SMILES: CCCCS(=O)c1sc2nc(-c3cccnc3)nc(-c3nccn3C)c2c1N] | | | 1.1 |
| | CCCCS(═O)C1═C(N)C2═C(N═C(N═C2S1)C1═CC═CN═C1) | | | |
| | C1═NC═CN1C | | | |
| 86B | 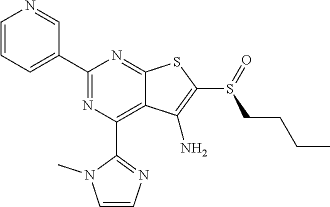 [SMILES: CCCC[S@](=O)c1sc2nc(-c3cccnc3)nc(-c3nccn3C)c2c1N] | (−) | | 41 |
| | CCCC[S@](═O)C1═C(N)C2═C(N═C(N═C2S1) | | | |
| | C1═CC═CN═C1)C1═NC═CN1C | | | |
| 86A | 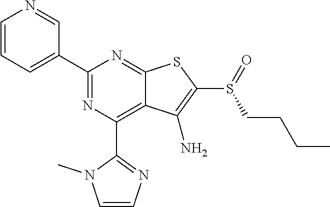 [SMILES: CCCC[S@@](=O)c1sc2nc(-c3cccnc3)nc(-c3nccn3C)c2c1N] | (+) | | 0.56 |
| | CCCC[S@@](═O)C1═C(N)C2═C(N═C(N═C2S1) | | | |
| | C1═CC═CN═C1)C1═NC═CN1C | | | |
| 87 | 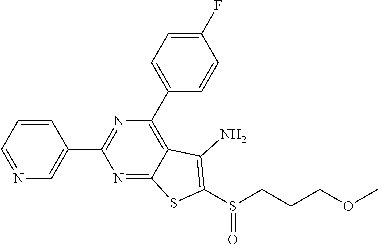 [SMILES: COCCCS(=O)c1sc2nc(-c3cccnc3)nc(-c3ccc(F)cc3)c2c1N] | | | Not tested |
| | COCCCS(═O)C1═C(N)C2═C(N═C(N═C2S1) | | | |
| | C1═CN═CC═C1)C1═CC═C(F)C═C1 | | | |
| 87B | 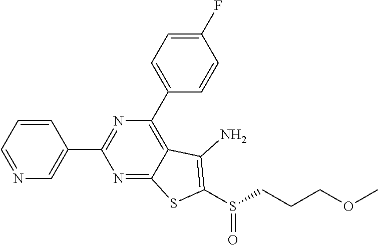 [SMILES: COCCC[S@](=O)c1sc2nc(-c3cccnc3)nc(-c3ccc(F)cc3)c2c1N] | (−) | | inactive |
| | COCCC[S@@](C1═C(C2═C(C3═CC═C(C═C3) | | | |
| | F)N═C(C4═CN═CC═C4)N═C2S1)N)═O | | | |
| 87A | 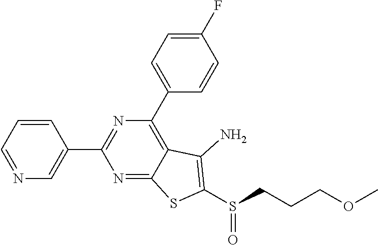 [SMILES: COCCC[S@@](=O)c1sc2nc(-c3cccnc3)nc(-c3ccc(F)cc3)c2c1N] | (+) | | 1.3 |
| | COCCC[S@](C1═C(C2═C(C3═CC═C(C═C3) | | | |
| | F)N═C(C4═CN═CC═C4)N═C2S1)N)═O | | | |
| 88 | 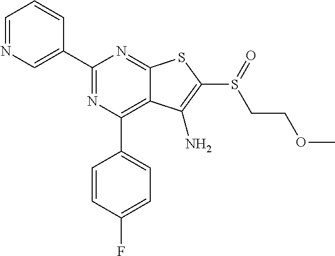 [SMILES: COCCS(=O)c1sc2nc(-c3cccnc3)nc(-c3ccc(F)cc3)c2c1N] | | | |
| | COCCS(═O)C1═C(N)C2═C(N═C(N═C2S1)C1═CN═CC═C1) | | | |
| | C1═CC═C(F)C═C1 | | | |
| 88B | 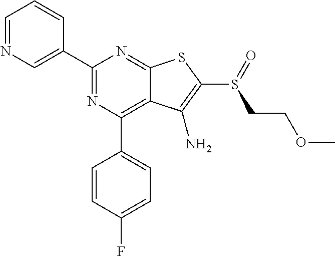 [SMILES: COCC[S@](=O)c1sc2nc(-c3cccnc3)nc(-c3ccc(F)cc3)c2c1N] | | | |
| | COCC[S@@](C1═C(C2═C(C3═CC═C(C═C3) | | | |
| | F)N═C(C4═CN═CC═C4)N═C2S1)N)═O | | | |
| 88A | 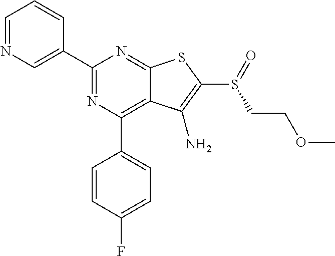 [SMILES: COCC[S@@](=O)c1sc2nc(-c3cccnc3)nc(-c3ccc(F)cc3)c2c1N] | | | |
| | COCC[S@](C1═C(C2═C(C3═CC═C(C═C3)F) | | | |
| | N═C(C4═CN═CC═C4)N═C2S1)N)═O | | | |
| 89 | 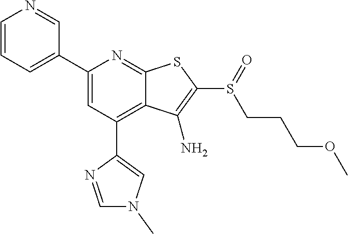 [SMILES: COCCCS(=O)c1sc2nc(-c3cccnc3)cc(-c3cn(C)cn3)c2c1N] | | | 1.3 |
| | CN1C═NC(C2═C3C(SC/S(CCCOC)═O)═C3N) | | | |
| | ═NC(C4═CC┘CN═C4)═C2)═C1 | | | |
| 89A | 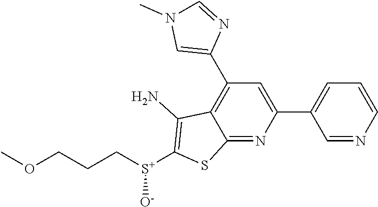 [SMILES: COCCC[S@@+]([O-])c1sc2nc(-c3cccnc3)cc(-c3cn(C)cn3)c2c1N] | | (+) | 0.77 |
| | CN(C═N1)C50 C1C(C═C(N═C2S3)C4═CC═CN═C4) | | | |
| | ═C2C(N)═C3[S@+]([O—])CCCOC | | | |
| 89B | 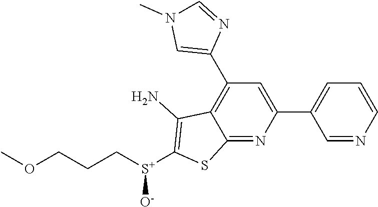 [SMILES: COCCC[S@+]([O-])c1sc2nc(-c3cccnc3)cc(-c3cn(C)cn3)c2c1N] | | (−) | 74 |
| | CN(C═N1)C═C1C(C═C(N═C2S3)C4═CC═CN═C4) | | | |
| | ═C2C(N)═C3[S@@+]([O—])CCCOC | | | |
| 90 | 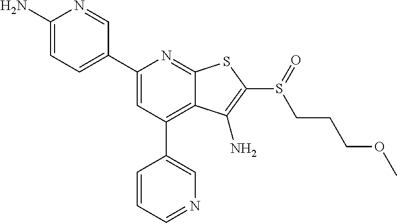 [SMILES: COCCCS(=O)c1sc2nc(-c3ccc(N)nc3)cc(-c3cccnc3)c2c1N] | | | 2.5 |
| | O═S(C1═C(C2═C(C3═CN═CC═C3)C═C(C4═CC═C | | | |
| | (N)N═C4)N═C2S1)N)CCCOC | | | |
| 91 | 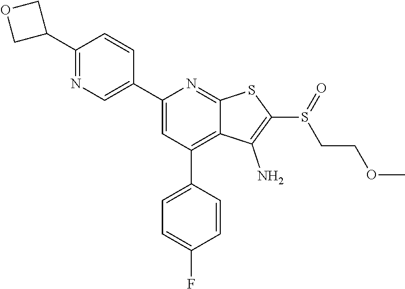 [SMILES: COCCS(=O)c1sc2nc(-c3ccc(C4COC4)nc3)cc(-c3ccc(F)cc3)c2c1N] | | | 2.4 |
| | FC1═CC═C(C2═C3C(SC(S(CCOC)═O)═C3N) | | | |
| | ═NC(C4═CN═C(C5COC5)C═C4)═C2)C═C1 | | | |

### Example 3

Table 2 provides a comparison of activity and metabolic stability of the thiazole containing 15-PGDH inhibitor, 2-(butylsulfinyl)-4-(1,2-dimethyl-1H-imidazol-5-yl)-6-(thiazol-2-yl)thieno[2,3-b]pyridin-3-amine, to various 6-membered ring (e.g., pyridine and pyrimidine) containing 15-PGDH inhibitors.

The activity (EC50 in nM) of the compounds was determined for induction of PGE2 from IL1-β treated A549 cells, as assayed in a medium of A549 cells that have been stimulated with by IL1-β for 24 hours. The metabolic stability was determined by incubating the compounds at a concentration of 10 μM in the presence of microsomes derived from mouse livers. Compound concentrations were monitored by HPLC/MS over time, and half-lives were calculated from the slope of the best-fit line for Ln[compound] vs time.

TABLE 2

| | | Metabolism |
|---|---|---|
| | induction | by mouse |
| | PGE2 in A549 | liver |
| | cells (EC50), | microsomes |
| Structure | nM) | (T1/2, min) |
| 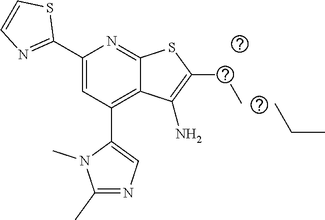 | 50 | 5 |
| [O—] | | |
| [S@@+](C1═C(C2═C(C═C(N═C2S1) | | |
| C3═NC═CS3)C4═CN═C(N4C)C)N)CCCC | | |
| 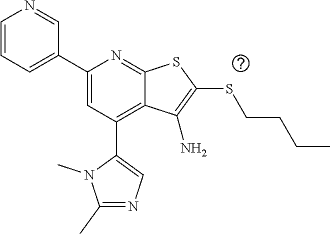 [SMILES: CCCCSc1sc2nc(-c3cccnc3)cc(-c3cnc(C)n3C)c2c1N] | <20 | 18 |
| [O—] | | |
| [S@+](CCCC)C1═C(N) | | |
| C2═C(C3═CN═C(C)N3C) | | |
| C═C(C4═CC═CN═C4)N═C2S1 | | |
| 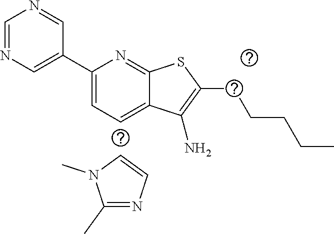 | <20 | |
| [O—] | | |
| [S@+](CCCC)C1═C(N)C2═C | | |
| (C3═CN═C(C)N3C)C═C | | |
| (C4═CN═CN═C4)N═C2S1 | | |
| 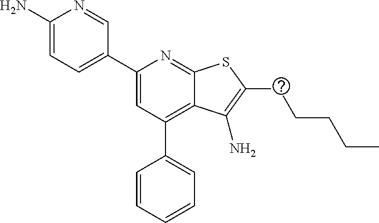 [SMILES: CCCCCc1sc2nc(-c3ccc(N)nc3)cc(-c3ccccc3)c2c1N] | <4 | 11 |
| CCCC[S@@+]([O—]) | | |
| C1═C(N)C2═C(C═C(N═C2S1) | | |
| C3═CC═C(N═C3)N)C4═CC═CC═C4 | | |
| 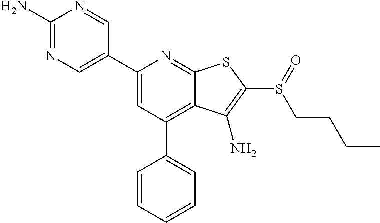 [SMILES: CCCCS(=O)c1sc2nc(-c3cnc(N)nc3)cc(-c3ccccc3)c2c1N] | <4 | |
| NC1═C(S(CCCC)═O)SC2═NC(C3═CN═C | | |
| (N)N═C3)═CC(C4═CC═CC═C4)═C21 | | |
| 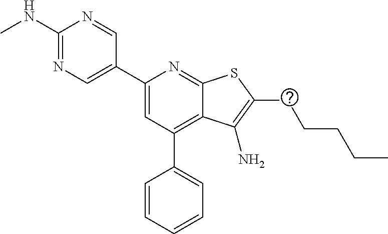 [SMILES: CCCCCc1sc2nc(-c3cnc(NC)nc3)cc(-c3ccccc3)c2c1N] | <4 | 12 |
| CCCC[S@@+]([O—]) | | |
| C1═C(N)C2═C(C═C(N═C2S1) | | |
| C3═CN═C(N═C3)NC)C4═CC═CC═C4 | | |
| 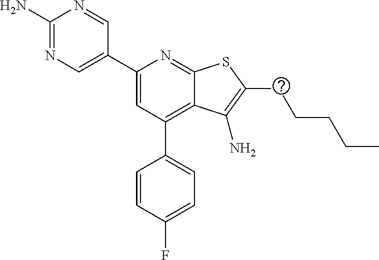 [SMILES: CCCCCc1sc2nc(-c3cnc(N)nc3)cc(-c3ccc(F)cc3)c2c1N] | 13 | 10 |
| CCCC[S@@+]([O—]) | | |
| C1═C(N)C2═C(C═C(N═C2S1) | | |
| C3═CN═C(N)N═C3)C4═CC═C(C═C4)F | | |

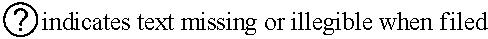

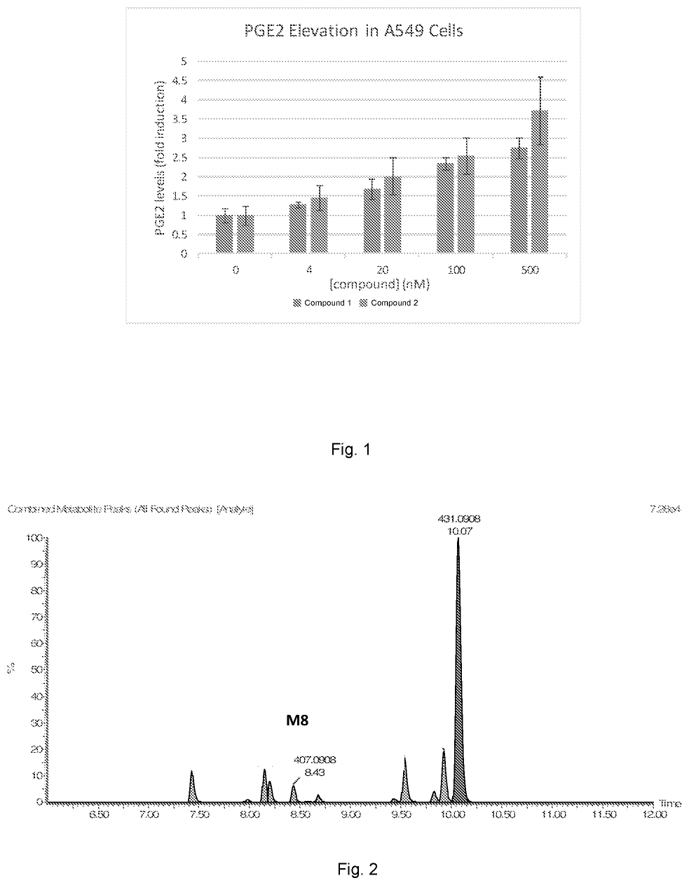

FIG. 1 illustrates a graph showing the dose response of compound 1 (2-(butylsulfinyl)-4-(2-cyclopropyl-1-methyl-1H-imidazol-5-yl)-6-(thiazol-2-yl)thieno[2,3-b]pyridin-3-amine) and compound 2 (compound 52A in Table 1; (R)-2-(butylsulfinyl)-4-(2-cylcopropyl-1-methyl-1H-imidazol-5-yl)-6-(pyridine-3-yl)thieno[2,3-b]pyridine-3-amine), on PGE2 production of A549 cells stimulated with IL-1.

FIG. 2 illustrates a liquid chromatograpy-mass spectrometry (LC-MS) chromatogram of 2-(butylsulfinyl)-4-(1,2-dimethyl-1H-imidazol-5-yl)-6-(thiazol-2-yl)thieno[2,3-b]pyridin-3-amine and its metabolites following in cubation in the presence of mouse liver microsomes.

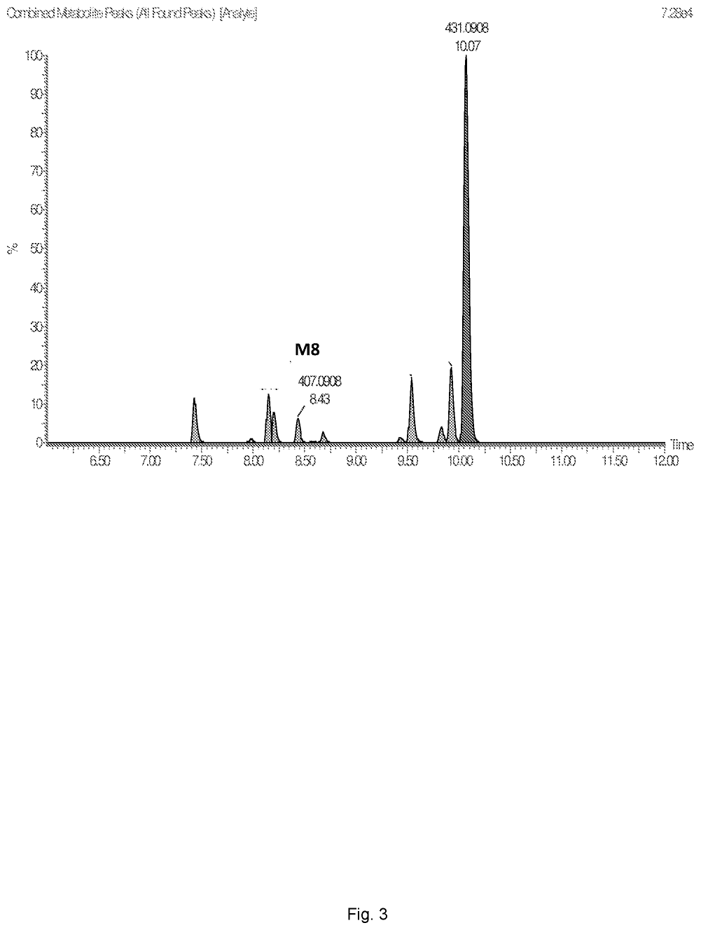

FIG. 3 illustrates a liquid chromatograpy-mass spectrometry (LC-MS) chromatogram of 2-(butylsulfinyl)-4-(1,2-dimethyl-1H-imidazol-5-yl)-6-(thiazol-2-yl)thieno[2,3-b]pyridin-3-amine and its metabolites following incubation in the presence of mouse liver microsomes.

FIGS. 2 and 3 show the formation of metabolites derived from oxidation of the thiazole metabolites from 2-(butylsulfinyl)-4-(1,2-dimethyl-1H-imidazol-5-yl)-6-(thiazol-2-yl)thieno[2,3-b]pyridin-3-amine (epoxy thiazoles and/or glyoxal). Metabolite M8 is a thioamide resulting from oxidative cleavage of the thiazole ring. By contrast, 6 membered ring heteroaryls cannot form this metabolite or the toxic byproducts. This data shows that 15-PGDH inhibitors described herein including 6-membered ring heterocycles provide higher activity in cells and improved metabolic stability relative to the 15-PGDH inhibitors which include a thiazole.

### Synthesis of SW209415: Representative Procedures

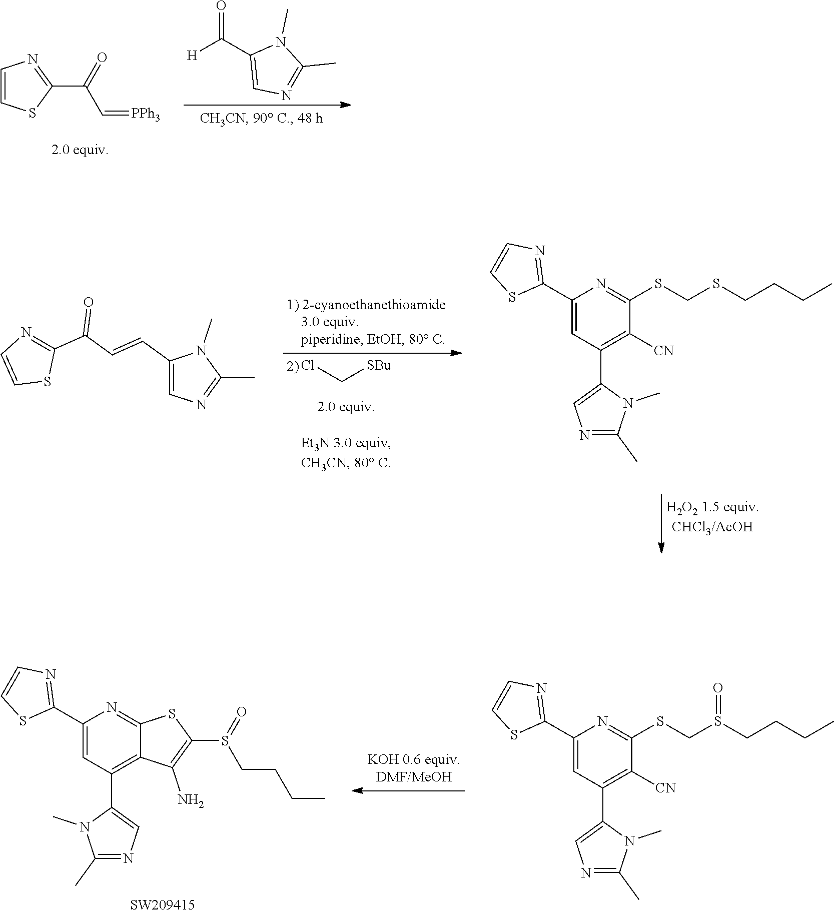

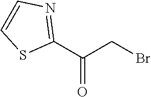

2-bromo-1-(thiazol-2-yl)ethan-1-one. n-BuLi (24.7 mL, 61.7 mmol, 2.5 M in Hexane) was added dropwise to a solution of 2-thiazole (5.0 g, 59 mmol) in anhydrous diethyl ether (50 mL) at −78° C. After 15 minutes, ethylbromoacetate (6.84 mL, 61.7 mmol) was added, the cold bath was removed and the solution was allowed to warm to room temperature. The reaction mixture was treated with AcOH (7 mL) and then diluted with water (100 mL) and ether (60 mL). The organic layer was separated, dried over Na₂SO₄, filtered and concentrated under reduced pressure. The crude product was suspended in hexanes and heated to reflux for 15 minutes then the product was decanted off leaving the impure oil. This was repeated 5 times to give a white solid with 88% yield. ¹H NMR (400 MHz, CDCl₃) δ 8.05 (d, J=3.0 Hz, 1H), 7.77 (d, J=3.0 Hz, 1H), 4.71 (s, 2H).

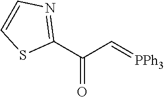

1-(thiazol-2-yl)-2-(triphenyl-λ-5-phosphanylidene)ethan-1-one. To a solution of 2-bromo-1-(thiazol-2-yl)ethan-1-one (10.7 g, 0.0517 mol) in toluene (337.7 mL), triphenylphosphine (14.1 g, 0.0539 mol) was added portion wise. The mixture was stirred at room temperature for 3 hours. The yellowish precipitate was removed by filtration, and was washed several times with toluene and then petroleum ether. Water was added to the precipitate and was treated dropwise with 1N NaOH to pH 10 (at pH 7 there was a color change from yellow to orange). The mixture was stirred for 30 minutes at room temperature. The precipitate was removed by filtration and washed several times with water. The resulting orange solid, was heated at 50° C. under vacuum to remove any water, giving a 96% yield. ¹H NMR (400 MHz, CDCl₃) δ 7.82 (d, J=3.1 Hz, 1H), 7.72 (ddd, J=12.8, 8.3, 1.4 Hz, 6H), 7.61-7.54 (m, 3H), 7.51-7.45 (m, 6H), 7.38 (dd, J=3.1, 1.3 Hz, 1H), 5.00 (d, J=23.3 Hz, 1H). EST-MS (m/z): 387.9 [M+H]⁺

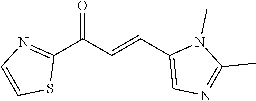 [SMILES: Cc1ncc(/C=C/C(=O)c2nccs2)n1C]

(E)-3-(1,2-dimethyl-1H-imidazol-5-yl)-1-(thiazol-2-yl)prop-2-en-1-one. To a solution of 1,5-dimethyl-1H-imidazole-2-carbaldehyde (2.0 mmol, 250 mg) in 6 ml of CH₃CN was added 1-(thiazol-2-yl)-2-(triphenyl-λ5-phosphanylidene)ethan-1-one (4.0 mmol, 1.55 g, 2.0 equiv.). The reaction mixture was stirred at 90° C. for 48 h. Once complete, solvent was evaporated and residue was purified by flash chromatography to give 331 mg of designed product (71%). ¹H NMR (400 MHz, CD₃OD) δ 8.08 (d, J=3.0 Hz, 1H), 7.97 (d, J=3.0 Hz, 1H), 7.90 (d, J=15.9 Hz, 1H), 7.76 (d, J=15.9 Hz, 1H), 7.60 (s, 1H), 3.72 (s, 3H), 2.43 (s, 3H). ESI-MS (m/z): 234.3 [M+H]⁺

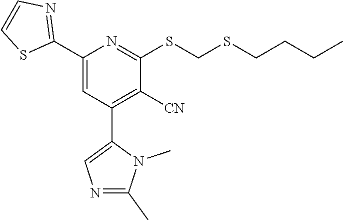 [SMILES: CCCCSCSc1nc(-c2nccs2)cc(-c2cnc(C)n2C)c1C#N]

2-(((butylthio)methyl)thio)-4-(1,2-dimethyl-1H-imidazol-5-yl)-6-(thiazol-2-yl)nicotinonitrile. To a suspension of 3-(1,2-dimethyl-1H-imidazol-5-yl)-1-(thiazol-2-yl)prop-2-en-1-one (0.31 mmol, 72 mg) and 2-cyanothioacetamide (0.93 mmol, 93 mg, 3.0 equiv.) in EtOH (1.5 mL) 3 drops of piperidine were added. After being stirred at 80° C. for 2 h, EtOH was evaporated and crude product was redissolved in 2 mL of CH₃CN. Butyl(chloromethyl)sulfane (0.62 mmol, 85.5 mg, 20 equiv.) and Et₃N (0.93 mmol, 94.1 mg, 130 μL) were then added and the reaction mixture was stirred at 80° C. for 20 min. Once complete, the reaction was diluted with EtOAc and water. The organic phase was separated and aqueous layer was extracted twice with EtOAc. The combined extractions were washed with saturated NaCl solution, dried over magnesium sulfate, filtered and concentrated under reduced pressure. The residue was purified by flash chromatography to give 99 mg of designed product (77%). ¹H NMR (400 MHz, CDCl₃) δ 7.96 (d, J=3.1 Hz, 1H), 7.85 (s, 1H), 7.56 (d, J=3.1 Hz, 1H), 7.37 (s, 1H), 4.49 (s, 2H), 3.60 (s, 3H), 2.72 (t, J=7.4 Hz, 2H), 2.48 (s, 3H), 1.62 (p, J=7.3 Hz, 2H), 1.40 (h, J=7.3 Hz, 2H), 0.90 (t, J=7.3 Hz, 3H). ESI-MS (m/z): 416.6 [M+H]⁺

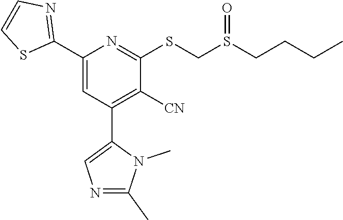 [SMILES: CCCCS(=O)CSc1nc(-c2nccs2)cc(-c2cnc(C)n2C)c1C#N]

2-(((butylsulfinyl)methyl)thio)-4-(1,2-dimethyl-1H-imidazol-5-yl)-6-(thiazol-2-yl)nicotinonitrile. To the solution of 2-(((butylthio)methyl)thio)-4-(1,2-dimethyl-1H-imidazol-5-yl)-6-(thiazol-2-yl)nicotinonitrile (85 mg, 0.205 mmol) in CHCl3/AcOH (1:1, 0.15 M) was added H₂O₂ (0.31 mmol, 1.5 equiv. 30% solution in water). The reaction mixture was stirred at 32° C. for 40 min. Once complete, the reaction was diluted with EtOAc and was washed with saturated NaHCO₃ solution, dried over magnesium sulfate, filtered and concentrated under reduce pressure to give designed product in 92% yield. ¹H NMR (400 MHz, CDCl3) δ 7.98 (d, J=3.1 Hz, 1H), 7.94 (s, 1H), 7.60 (d, J=3.1 Hz, 1H), 7.43 (s, 1H), 4.72 (d, J=13.1 Hz, 1H), 4.41 (d, J=13.1 Hz, 1H), 3.63 (s, 3H), 2.96 (dt, J=12.9, 8.2 Hz, 1H), 2.84 (dt, J=12.9, 7.5 Hz, 1H), 2.51 (s, 3H), 1.94-1.74 (m, 2H), 1.63-1.38 (m, 2H), 0.95 (t, J=7.4 Hz, 3H). ESI-MS (m/z): 432.1 [M+H]⁺.

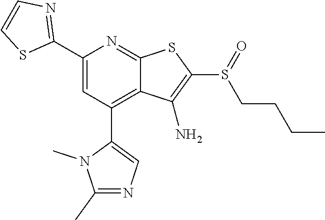 [SMILES: CCCCS(=O)c1sc2nc(-c3nccs3)cc(-c3cnc(C)n3C)c2c1N]

SW209415. 2-(butylsulfinyl)-4-(1,2-dimethyl-1H-imidazol-5-yl)-6-(thiazol-2-yl)thieno[2,3-b]pyridin-3-amine. To the solution of 2-(((butylsulfinyl)methyl)thio)-4-(1,2-dimethyl-1H-imidazol-5-yl)-6-(thiazol-2-yl)nicotinonitrile (0.14 mmol, 60 mg) in DMF (600 μl)/MeOH (300 μl) was added KOH (0.084 mmol, 4.70 mg, 0.6 equiv., 2.0 M in water). The reaction mixture was stirred at 32° C. for 10 min. Once complete, the reaction was diluted with EtOAc and acidified to pH 7 with 5% aq. solution of AcOH, the organic phase was separated and aqueous layer was extracted twice with EtOAc, dried over magnesium sulfate, filtered and concentrated under reduced pressure. The crude product was purified by flash chromatography to afford designed product in 97% isolated yield. ¹H NMR (400 MHz, CDCl₃) δ 8.03 (s, 1H), 7.90 (d, J=3.1 Hz, 1H), 7.50 (d, J=3.2 Hz, 1H), 7.11 (s, 1H), 4.76 (s, 2H), 3.39 (s, 3H), 3.27 (ddd, J=12.9, 8.7, 6.4 Hz, 1H), 3.09 (ddd, J=12.8, 8.8, 6.9 Hz, 1H), 2.47 (s, 3H), 1.83-1.62 (m, 2H), 1.57-1.38 (m, 2H), 0.93 (t, J=7.3 Hz, 3H). ESI-MS (m/z): 432.1 [M+H]⁺. Two enantiomers of SW209415 can be separated by chiral HPLC: Chiralpak AD-H, 1×25 cm, 100% MeOH.

### Examples

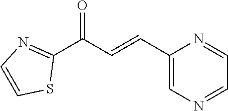 [SMILES: O=C(/C=C/c1cnccn1)c1nccs1]

Example 1-1: (E)-3-(pyrazin-2-yl)-1-(thiazol-2-yl)prop-2-en-1-one was prepared in a 74% yield from 1-(4-methylthiazol-2-yl)-2-(triphenyl-λ-5-phosphanylidene)ethan-1-one (1.0 equiv.) and pyrazine-2-carbaldehyde (1.0 equiv.) via Wittig reaction, described for the preparation of SW209415. ¹H NMR (400 MHz, CDCl₃) δ 8.81 (d, J=1.5 Hz, 1H), 8.67 (dd, J=2.3, 1.4 Hz, 1H), 8.58 (d, J=2.3 Hz, 1H), 8.48 (d, J=15.8 Hz, 1H), 8.10 (d, J=3.0 Hz, 1H), 8.01 (d, J=15.8, 1H), 7.76 (d, J=3.0 Hz, 1H). ESI-MS (m/z): 218.0 [M+H]⁺.

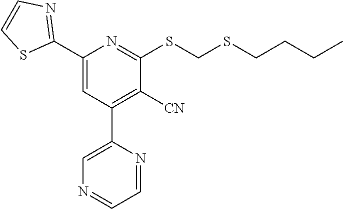 [SMILES: CCCCSCSc1nc(-c2nccs2)cc(-c2cnccn2)c1C#N]

Example 1-2: 2-(((butylthio)methyl)thio)-4-(pyrazin-2-yl)-6-(thiazol-2-yl)nicotinonitrile was prepared from (E)-3-(pyrazin-2-yl)-1-(thiazol-2-yl)prop-2-en-1-one and butyl(chloromethyl)sulfane in 8% yield, using synthetic procedures described for the preparation of SW209415. ¹H NMR (400 MHz, CDCl₃) δ 9.15 (d, J=1.4 Hz, 1H), 8.81 (dd, J=2.4, 1.4 Hz, 1H), 8.76 (d, J=2.4 Hz, 1H), 8.32 (s, 1H), 8.02 (d, J=3.1 Hz, 1H), 7.61 (d, J=3.1 Hz, 1H), 4.54 (s, 2H), 2.77 (t, J=7.4 Hz, 3H), 1.69-1.60 (m, 2H), 1.48-1.39 (m, 2H), 0.92 (t, J=7.4 Hz, 3H). ESI-MS (m/z): 400.1 [M+H]⁺.

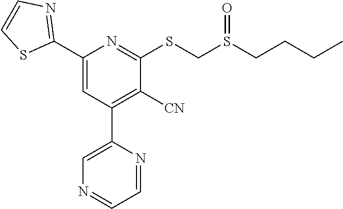 [SMILES: CCCCS(=O)CSc1nc(-c2nccs2)cc(-c2cnccn2)c1C#N]

Example 1-3: 2-(((butylsulfinyl)methyl)thio)-4-(pyrazin-2-yl)-6-(thiazol-2-yl)nicotinonitrile was prepared via standard oxidation reaction with hydrogen peroxide, using the synthetic procedures described for the preparation of SW209415. ¹H NMR (400 MHz, CDCl₃) δ 9.17 (s, 1H), 8.82 (s, 1H), 8.78 (s, 1H), 8.42 (s, 1H), 8.04 (d, J=2.7 Hz, 1H), 7.64 (d, J=2.7 Hz, 1H), 4.76 (d, J=13.0 Hz, 1H), 4.41 (d, J=13.1 Hz, 1H), 3.03-2.95 (dt, J=12.9, 8.1 Hz, 1H), 2.88-2.81 (dt, J=12.9, 7.3 Hz, 1H), 1.89-1.81 (m, 2H), 1.57-1.46 (m, 2H), 0.97 (t, J=7.3 Hz, 3H). ESI-MS (m/z): 416.1 [M+H]⁺.

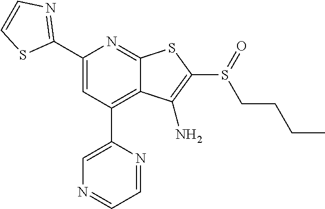 [SMILES: CCCCS(=O)c1sc2nc(-c3nccs3)cc(-c3cnccn3)c2c1N]

Example 1: 2-(butylsulfinyl)-4-(pyrazin-2-yl)-6-(thiazol-2-yl)thieno[2,3-b]pyridin-3-amine was prepared in 99% yield, via standard cyclization reaction with potassium hydroxide, using the synthetic procedures described for the preparation of SW209415. ¹H NMR (400 MHz, CD₂Cl₂) δ 9.11 (d, J=1.4 Hz, 1H), 8.78 (d, J=2.5 Hz, 1H), 8.76-8.70 (m, 1H), 8.32 (s, 1H), 7.97 (d, J=3.1 Hz, 1H), 7.59 (d, J=3.1 Hz, 1H), 5.59 (s, 2H), 3.30-3.23 (ddd, J=12.7, 10.2, 6.6 Hz, 1H), 3.16-3.08 (ddd, J=13.0, 9.8, 6.6 Hz, 1H), 1.77-1.68 (m, 2H), 1.54-1.45 (m, 2H), 0.94 (t, J=7.3 Hz, 3H). ESI-MS (m/z): 416.0 [M+H]⁺.

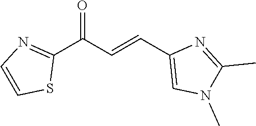 [SMILES: Cc1nc(/C=C/C(=O)c2nccs2)cn1C]

Example 2-1: (E)-3-(1,2-dimethyl-1H-imidazol-4-yl)-1-(thiazol-2-yl)prop-2-en-1-one. To a solution of 1,2-dimethyl-1H-imidazole-4-carbaldehyde (1.3 mmol, 162 mg) in 2 ml of CH₃CN was added 1-(thiazol-2-yl)-2-(triphenyl-□5-phosphanylidene)ethan-1-one (2.6 mmol, 1.01 g, 2.0 equiv.). The reaction mixture was stirred at 90° C. for 48 h. Once complete, solvent was evaporated and residue was purified by flash chromatography. ESI-MS (m/z): 234.3 [M+H]⁺.

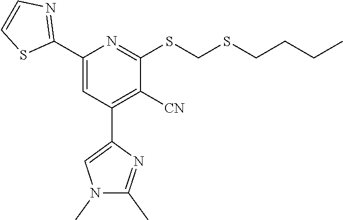 [SMILES: CCCCSCSc1nc(-c2nccs2)cc(-c2cn(C)c(C)n2)c1C#N]

Example 2-2: 2-(((butylthio)methyl)thio)-4-(1,2-dimethyl-1H-imidazol-4-yl)-6-(thiazol-2-yl)nicotinonitrile was prepared from (E)-3-(1,2-dimethyl-1H-imidazol-4-yl)-1-(thiazol-2-yl)prop-2-en-1-one, 2-cyanoethanethioamide and Butyl(chloromethyl)sulfane (2.0 equiv.) in 75% isolated yield, using synthetic procedures described for the preparation of SW209415. ¹H NMR (400 MHz, CDCl₃) δ 8.65 (s, 1H), 7.97 (d, J=3.1 Hz, 1H), 7.93 (s, 1H), 7.51 (d, J=3.2 Hz, 1H), 4.51 (s, 2H), 3.65 (s, 3H), 2.75 (t, J=7.4 Hz, 2H), 2.4 (s, 3H), 1.63 (tt, J=8.4, 6.8 Hz, 2H), 1.50-1.30 (m, 2H), 0.90 (t, J=7.3 Hz, 3H). ESI-MS (m/z): 416.1 [M+H]⁺.

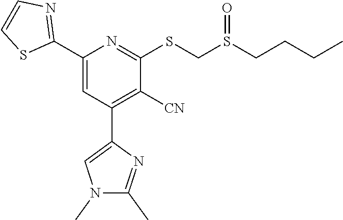 [SMILES: CCCCS(=O)CSc1nc(-c2nccs2)cc(-c2cn(C)c(C)n2)c1C#N]

Example 2-3: 2-(((butylsulfinyl)methyl)thio)-4-(1,2-dimethyl-1H-imidazol-4-yl)-6-(thiazol-2-yl)nicotinonitrile was prepared via standard oxidation reaction with hydrogen peroxide in 98% isolated yield, using synthetic procedures described for the preparation of SW209415. ¹H NMR (400 MHz, CDCl₃) δ 8.76-8.59 (m, 1H), 7.98 (d, J=3.1 Hz, 1H), 7.93 (s, 1H), 7.53 (d, J=3.1 Hz, 1H), 4.73 (d, J=13.1 Hz, 1H), 4.43 (d, J=13.1 Hz, 1H), 3.66 (s, 3H), 2.97 (dt, J=13.0, 8.2 Hz, 1H), 2.91-2.76 (m, 1H), 2.44 (s, 3H), 1.92-1.70 (m, 2H), 1.62-1.33 (m, 2H), 0.93 (t, J=7.3 Hz, 3H).

ESI-MS (m/z): 432.1 [M+H]⁺.

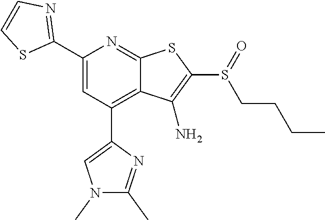 [SMILES: CCCCS(=O)c1sc2nc(-c3nccs3)cc(-c3cn(C)c(C)n3)c2c1N]

Examples 2, 3 and 4. 2-(butylsulfinyl)-4-(1,2-dimethyl-1H-imidazol-4-yl)-6-(thiazol-2-yl)thieno[2,3-b]pyridin-3-amine was prepared via cyclization reaction in 67% isolated yield, using synthetic procedures described for the preparation of SW209415. ¹H NMR (400 MHz, CD₂Cl₂) δ 8.24 (s, 1H), 7.94 (d, J=3.2 Hz, 1H), 7.55 (s, 1H), 7.53 (d, J=3.2 Hz, 1H), 7.42 (s, 2H), 3.67 (s, 3H), 3.26 (ddd, J=12.7, 9.3, 5.8 Hz, 1H), 3.11 (ddd, J=12.7, 9.4, 6.2 Hz, 1H), 2.45 (s, 3H), 1.79-1.59 (m, 2H), 1.59-1.39 (m, 2H), 0.95 (t, J=7.3 Hz, 3H). ESI-MS (m/z): 432.1 [M+H]⁺. Enantiomers were separated on a 1 cm Chiralpak OD column using 20% iPrOH and 80% Hex. with 10 mL/min flow rate, 350 μL injection (concentration 10 mg/ml) the 1st peak (Example 4) was at 30 min and the 2nd peak (Example 3) was at 33 min. Optical Rotation: Peak 1 [□] +111 (c=0.39, EtOH), Peak 2 [□] −108 (c=0.46, EtOH).

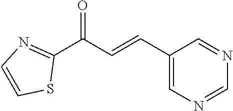 [SMILES: O=C(/C=C/c1cncnc1)c1nccs1]

Example 5-1: (E)-3-(pyrimidin-5-yl)-1-(thiazol-2-yl)prop-2-en-1-one was prepared via Wittig reaction of 1-(4-methylthiazol-2-yl)-2-(triphenyl-λ-5-phosphanylidene)ethan-1-one (1.0 equiv.) and pyrimidine-5-carbaldehyde (1.0 equiv.) in 32% yield, using synthetic described for the preparation of SW209415. ¹H NMR (400 MHz, CDCl₃) δ 9.24 (s, 1H), 9.04 (s, 2H), 8.10 (d, J=16.2 Hz, 1H), 8.10 (d, J=2.9 Hz, 1H), 7.92 (d, J=16.3 Hz, 1H), 7.78 (d, J=2.9 Hz, 1H). ESI-MS (m/z): 218.0 [M+H]⁺.

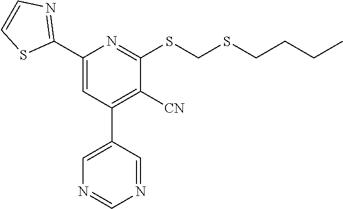 [SMILES: CCCCSCSc1nc(-c2nccs2)cc(-c2cncnc2)c1C#N]

Example 5-2: 2-(((butylthio)methyl)thio)-4-(pyrimidin-5-yl)-6-(thiazol-2-yl)nicotinonitrile was prepared in 51% yield from (E)-3-(pyrimidin-5-yl)-1-(thiazol-2-yl)prop-2-en-1-one and butyl(chloromethyl)sulfane, using synthetic procedures described for the preparation of SW209415. ¹H NMR (400 MHz, CDCl₃) δ 9.39 (s, 1H), 9.05 (s, 2H), 8.04 (s, 1H), 8.02 (d, J=3.1 Hz, 1H), 7.63 (d, J=3.1 Hz, 1H), 4.54 (s, 2H), 2.78 (t, J=7.4 Hz, 3H), 1.70-1.62 (m, 2H), 1.49-1.40 (m, 2H), 0.93 (t, J=7.3 Hz, 3H). ESI-MS (m/z): 400.1 [M+H]⁺.

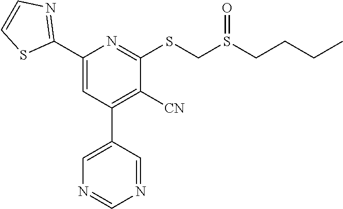 [SMILES: CCCCS(=O)CSc1nc(-c2nccs2)cc(-c2cncnc2)c1C#N]

Example 5-3: 2-(((butylsulfinyl)methyl)thio)-4-(pyrimidin-5-yl)-6-(thiazol-2-yl)nicotinonitrile was prepared in 94% isolated yield via standard oxidation with hydrogen peroxide (1.0 equiv), using the synthetic procedures described for the preparation of SW209415. ¹H NMR (400 MHz, CDCl₃) δ 9.41 (s, 1H), 9.06 (s, 2H), 8.13 (s, 1H), 8.04 (d, J=3.0 Hz, 1H), 7.66 (d, J=3.0 Hz, 1H), 4.72 (d, J=13.1 Hz, 1H), 4.46 (d, J=13.2 Hz, 1H), 3.02-2.93 (m, 1H), 2.91-2.81 (m, 1H), 1.89-1.82 (m, 2H), 1.54-1.45 (m, 2H), 0.98 (t, J=7.3 Hz, 3H). EST-MS (m/z): 416.1 [M+H]⁺.

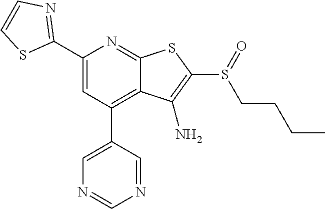 [SMILES: CCCCS(=O)c1sc2nc(-c3nccs3)cc(-c3cncnc3)c2c1N]

Example 5-4: 2-(butylsulfinyl)-4-(pyrimidin-5-yl)-6-(thiazol-2-yl)thieno[2,3-b]pyridin-3-amine was prepared via standard cyclization reaction of 2-(((butylsulfinyl)methyl)thio)-4-(pyrimidin-5-yl)-6-(thiazol-2-yl)nicotinonitrile with potassium hydroxide, using the synthetic procedures described for the preparation of the analog SW209415. It was observed in the crude reaction mixture (ESI-MS (m/z): 416.0 [M+H]⁺) but could not be isolated. Rather, it cyclized and oxidized as described below.

The desired product 2-(butylsulfinyl)-4-(pyrimidin-5-yl)-6-(thiazol-2-yl)thieno[2,3-b]pyridin-3-amine (Example 5-4) is unstable and cyclizes completely during 10 days to Example 6, (5-(butylsulfinyl)-2-(thiazol-2-yl)-6H-pyrimido[4,5-c]thieno[2,3,4-ij][2,7]naphthyridine) and Example 7, (5-(butylsulfinyl)-2-(thiazol-2-yl)-7H-pyrimido[4,5-c]thieno[2,3,4-ij][2,7]naphthyridine) with Example 6 being the major product. The cyclized products are separable on preparative HPLC: 21.2×250 mm Kinetex 5 μm EVO C18 100 Å reversed phase column using a 2:1 ratio of water to CH₃CN with a 10 mL/min flow rate and 100 μL injection volume.

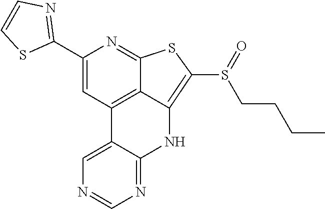 [SMILES: CCCCS(=O)c1sc2nc(-c3nccs3)cc3c2c1Nc1ncncc1-3]

Example 6: 5-(butylsulfinyl)-2-(thiazol-2-yl)-6H-pyrimido[4,5-c]thieno[2,3,4-ij][2,7]naphthyridine ¹H NMR (400 MHz, (CD₃)₂SO) δ 11.85 (s, 1H), 9.41 (s, 1H), 8.77 (s, 1H), 8.52 (s, 1H), 8.06 (d, J=3.1 Hz, 1H), 7.97 (d, J=3.1 Hz, 1H), 3.16-3.02 (m, 2H), 1.67-1.59 (m, 2H), 1.48-1.39 (m, 2H), 0.89 (t, J=7.3 Hz, 3H). ESI-MS (m/z): 414.0 [M+H]⁺.

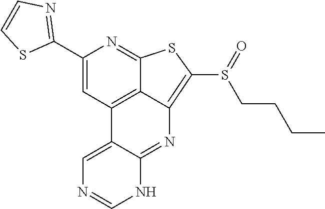 [SMILES: CCCCS(=O)c1sc2nc(-c3nccs3)cc3c4c(nc1c23)NC=NC=4]

Example 7: 5-(butylsulfinyl)-2-(thiazol-2-yl)-7H-pyrimido[4,5-c]thieno[2,3,4-ij][2,7]naphthyridine ¹H NMR (400 MHz, CD₂Cl₂) δ 10.08 (s, 1H), 9.56 (s, 1H), 8.74 (s, 1H), 7.97 (d, J=2.8 Hz, 1H), 7.52 (d, J=2.8 Hz, 1H), 7.41 (d, J=6.0 Hz, 1H), 3.21-3.08 (m, 2H), 1.95-1.85 (m, 1H), 1.82-1.72 (m, 1H), 1.53-1.46 (m, 2H), 0.98 (t, J=7.3 Hz, 3H). ESI-MS (m/z): 414.1 [M+H]⁺

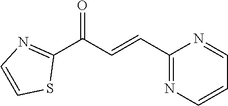 [SMILES: O=C(/C=C/c1ncccn1)c1nccs1]

Example 8-1: (E)-3-(pyrimidin-2-yl)-1-(thiazol-2-yl)prop-2-en-1-one was prepared in 74% yield, via Wittig reaction of 1-(4-methylthiazol-2-yl)-2-(triphenyl-λ-5-phosphanylidene)ethan-1-one (1.0 equiv.) and pyrimidine-2-carbaldehyde (1.0 equiv.), using synthetic procedures similar to those described for the preparation of the analog SW209415. ¹H NMR (400 MHz, CDCl₃) δ 8.88 (d, J=4.9 Hz, 2H), 8.57 (d, J=15.8 Hz, 1H), 8.15 (d, J=3.0 Hz, 1H), 8.05 (d, J=3.0 Hz, 1H), 7.89 (d, J=15.8 Hz, 1H), 7.45 (t, J=4.9 Hz, 1H). ESI-MS (m/z): 218.0 [M+H]⁺.

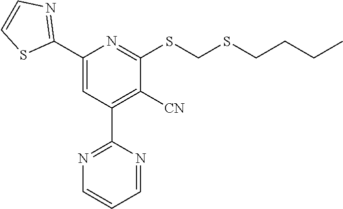 [SMILES: CCCCSCSc1nc(-c2nccs2)cc(-c2ncccn2)c1C#N]

Example 8-2: 2-(((butylthio)methyl)thio)-4-(pyrimidin-2-yl)-6-(thiazol-2-yl)nicotinonitrile was prepared in 9% yield from (E)-3-(pyrimidin-2-yl)-1-(thiazol-2-yl)prop-2-en-1-one and butyl(chloromethyl)sulfane, using synthetic procedures described for the preparation of the analog SW209415. ¹H NMR (400 MHz, CDCl₃) δ 8.97 (d, J=4.9 Hz, 2H), 8.85 (s, 1H), 8.03 (d, J=3.1 Hz, 1H), 7.58 (d, J=3.1 Hz, 1H), 7.43 (t, J=4.9 Hz, 1H), 4.53 (s, 2H), 2.76 (t, J=7.4, 2H), 1.67-1.61 (m, 2H), 1.48-1.38 (m, 2H), 0.92 (t, J=7.3 Hz, 3H). EST-MS (m/z): 400.1 [M+H]⁺.

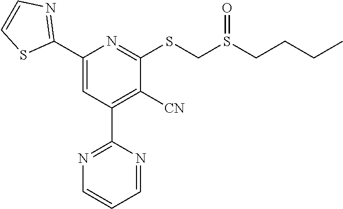 [SMILES: CCCCS(=O)CSc1nc(-c2nccs2)cc(-c2ncccn2)c1C#N]

Example 8-3: 2-(((butylsulfinyl)methyl)thio)-4-(pyrimidin-2-yl)-6-(thiazol-2-yl)nicotinonitrile was prepared in 91% yield via standard oxidation reaction with hydrogen peroxide, using the synthetic procedures described for the preparation of the analog SW209415. ¹H NMR (400 MHz, CDCl₃) δ 8.98 (d, J=4.9 Hz, 2H), 8.95 (s, 1H), 8.04 (d, J=3.1 Hz, 1H), 7.61 (d, J=3.1 Hz, 1H), 7.45 (t, J=4.9 Hz, 1H), 4.80 (d, J=13.1 Hz, 1H), 4.38 (d, J=13.1 Hz, 1H), 3.04-2.97 (dt, J=12.9, 8.1 Hz, 1H), 2.82 (dt, J=13.0, 7.2 Hz, 1H), 1.89-1.80 (m, 2H), 1.57-1.43 (m, 2H), 0.96 (t, J=7.3 Hz, 3H). ESI-MS (m/z): 416.1 [M+H]⁺.

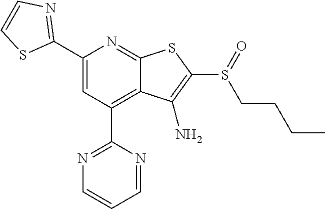 [SMILES: CCCCS(=O)c1sc2nc(-c3nccs3)cc(-c3ncccn3)c2c1N]

Example 8: 2-(butylsulfinyl)-4-(pyrimidin-2-yl)-6-(thiazol-2-yl)thieno[2,3-b]pyridin-3-amine was prepared in 30% isolated yield via standard cyclization reaction with potassium hydroxide, using the synthetic procedures described for the preparation of the analog SW209415. ¹H NMR (400 MHz, CD₂Cl₂) δ 9.00 (d, J=4.9 Hz, 2H), 8.87 (s, 1H), 7.99 (d, J=3.0 Hz, 1H), 7.57 (d, J=3.0 Hz, 1H), 7.48 (t, J=4.9 Hz, 1H), 6.41 (s, 2H), 3.31-3.25 (ddd, J=12.7, 9.8, 6.2 Hz, 1H), 3.18-3.10 (ddd, J=13.5, 10.5, 6.5 Hz, 1H), 1.77-1.70 (m, 2H), 1.53-1.44 (m, 2H), 0.95 (t, J=7.3 Hz, 3H). ESI-MS (m/z): 416.1 [M+H]⁺.

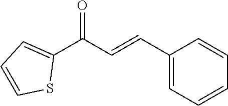 [SMILES: O=C(/C=C/c1ccccc1)c1cccs1]

Example 10-1: 3-phenyl-1-(thiophen-2-yl)prop-2-en-1-one was prepared from benzaldehyde and 1-(thiophen-2-yl)ethanone via aldol condensation using the procedure described by Azam. ¹H NMR (400 MHz, CDCl₃) δ 7.88-7.80 (m, 2H), 7.67 (dd, J=4.9, 1.1 Hz, 1H), 7.66-7.59 (m, 2H), 7.47-7.34 (m, 4H), 7.18 (dd, J=5.0, 3.8 Hz, 1H). ESI-MS (m/z): 215.1 [M+H]⁺

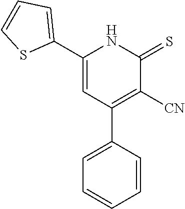 [SMILES: N#Cc1c(-c2ccccc2)cc(-c2cccs2)[nH]c1=S]

Example 10-2: 4-phenyl-6-(thiophen-2-yl)-2-thioxo-1,2-dihydropyridine-3-carbonitrile. To a solution of 3-phenyl-1-(thiophen-2-yl)prop-2-en-1-one (2.34 mmol, 500 mg) and cyanothioacetamide (7.0 mmol, 717 mg, 3.0 equiv.) in ethanol (7 mL), a few drops of piperidine were added. The reaction was refluxed for 3 h. The solid that formed was collected, suspended in acetic acid and heated at 80° C. After 30 min of heating, the mixture was cooled to room temperature and filtered to give the desired product in 46% isolated yield. ¹H NMR (400 MHz, DMSO-d₆) δ 8.17 (d, J=3.8 Hz, 1H), 7.96 (d, J=5.0 Hz, 1H), 7.74-7.62 (m, 2H), 7.54 (dd, J=5.1, 2.0 Hz, 3H), 7.31-7.19 (m, 1H), 7.01 (s, 1H). ESI-MS (m/z): 295.1 [M+H]⁺

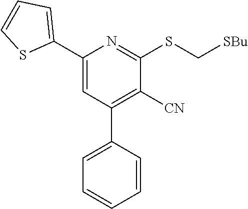

Example 10-3: 2-(((butylthio)methyl)thio)-4-phenyl-6-(thiophen-2-yl)nicotinonitrile. A mixture of 4-phenyl-6-(thiophen-2-yl)-2-thioxo-1,2-dihydropyridine-3-carbonitrile (0.34 mmol, 101 mg), butyl(chloromethyl)sulfane (0.34 mmol, 48 mg, 1.0 equiv.) and Et₃N (0.51 mmol, 0.07 mL, 1.5 equiv.) was refluxed in dry CH₃CN (0.35 mL) for 20 min. The reaction mixture was then diluted with EtOAc and water. The organic phase was separated and aqueous layer was extracted twice with EtOAc. The combined extractions were washed with saturated NaCl solution, dried over magnesium sulfate, filtered and concentrated under reduced pressure. The residue was purified by flash chromatography to give 124 mg of desired product (92%). ¹H NMR (400 MHz, CDCl₃) δ 7.70 (dd, J=3.8, 1.1 Hz, 1H), 7.64-7.56 (m, 1H), 7.55-7.47 (m, 5H), 7.40 (d, J=1.1 Hz, 1H), 7.14 (dd, J=5.0, 3.8 Hz, 1H), 4.53 (s, 2H), 2.74 (t, J=8.0 Hz, 2H), 1.72-1.57 (m, 2H), 1.49-1.34 (m, 2H), 0.90 (t, J=7.4 Hz, 3H). ¹³C NMR (101 MHz, CDCl₃) δ 163.0, 154.4, 153.7, 143.2, 135.9, 130.5, 130.1, 129.0, 128.6, 128.3, 127.2, 115.6, 114.1, 114.1, 103.2, 34.5, 32.1, 31.3, 22.0, 13.7. EST-MS (m/z): 397.1 [M+H]⁺.

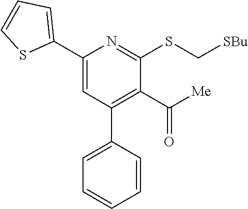

Example 10-4: 1-(2-(((butylthio)methyl)thio)-4-phenyl-6-(thiophen-2-yl)pyridin-3-yl)ethan-1-one. To the solution of 2-(((butylthio)methyl)thio)-4-phenyl-6-(thiophen-2-yl)nicotinonitrile (150 mg, 0.38 mmol) in THF (500 μL) was added MeLi (0.90 mmol, 600 μL, 1.5 M) and the reaction was stirred at −78° C. for 2 h. Once completed, the reaction was quenched with a saturated solution of NH₄Cl and diluted with EtOAc. The organic phase was separated and aqueous layer was extracted twice with EtOAc. The combined extractions were washed with saturated NaCl solution, dried over MgSO₄, filtered and concentrated under reduced pressure. The crude product was dissolved in CHCl₃ (1.0 mL) and AcOH (1.0 mL) and stirred overnight at 50° C. The reaction mixture was then diluted with EtOAc and sat solution of NaHCO₃. The organic phase was separated and aqueous layer was extracted twice with EtOAc. The combined extractions were dried over magnesium sulfate, filtered and concentrated under reduced pressure. The residue was purified by flash chromatography to give 140 mg of desired product (89%). ¹H NMR (400 MHz, CDCl₃) δ 7.69-7.60 (m, 1H), 7.57-7.34 (m, 7H), 7.18-7.07 (m, 1H), 4.47 (s, 2H), 2.72 (t, J=7.3 Hz, 2H), 1.96 (s, 3H), 1.71-1.54 (m, 2H), 1.48-1.33 (m, 2H) 0.89 (t, J=7.4 Hz, 3H). ESI-MS (m/z): 414.1 [M+H]⁺.

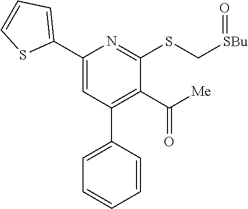

Example 10-5: 1-(2-(((butylsulfinyl)methyl)thio)-4-phenyl-6-(thiophen-2-yl)pyridin-3-yl)ethan-1-one was prepared in 97% isolated yield via standard oxidation reaction with hydrogen peroxide described for the preparation of analog SW209415. ¹H NMR (400 MHz, CDCl₃) δ 7.70 (dt, J=3.7, 0.9 Hz, 1H), 7.61-7.44 (m, 4H), 7.43 (s, 1H), 7.42-7.35 (m, 2H), 7.14 (ddd, J=5.0, 3.7, 0.6 Hz, 1H), 4.70 (d, J=12.9 Hz, 1H), 4.33 (d, J=12.9 Hz, 1H), 2.98 (dt, J=12.9, 8.2 Hz, 1H), 2.85-2.70 (m, 1H), 1.93 (s, 3H), 1.88-1.77 (m, 2H), 1.57-1.38 (m, 2H), 0.94 (t, J=7.3 Hz, 3H). EST-MS (m/z): 430.1 [M+H]⁺

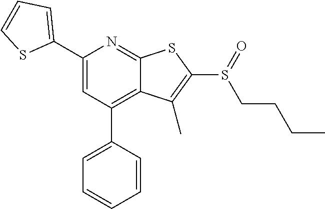 [SMILES: CCCCS(=O)c1sc2nc(-c3cccs3)cc(-c3ccccc3)c2c1C]

Example 10: 2-(butylsulfinyl)-3-methyl-4-phenyl-6-(thiophen-2-yl)thieno[2,3-b]pyridine To the solution of 1-(2-(((butylsulfinyl)methyl)thio)-4-phenyl-6-(thiophen-2-yl)pyridin-3-yl)ethan-1-one (0.06 mmol, 27 mg) in DMF 600 μL/MeOH 300 μL was added KOH (0.09 mmol, 5.4 mg, 1.5 equiv., 1.7 M in water). The reaction mixture was stirred at 50° C. for 10 min. Once complete (the reaction was followed by TLC and LCMS), the reaction was diluted with EtOAc and washed with 10% aq. solution of AcOH, the organic phase was separated and aqueous layer was extracted twice with EtOAc, dried over magnesium sulfate, filtered and concentrated under reduced pressure to give 2-(butylsulfinyl)-3-methyl-4-phenyl-6-(thiophen-2-yl)thieno[2,3-b]pyridine in 62% yield. ¹H NMR (400 MHz, CD₂Cl₂) δ 7.78-7.64 (m, 1H), 7.59 (s, 1H), 7.56-7.31 (m, 6H), 7.28-7.05 (m, 1H), 3.17 (ddd, J=12.9, 9.0, 6.1 Hz, 1H), 2.98 (ddd, J=12.9, 9.1, 6.7 Hz, 1H), 1.92 (s, 3H), 1.79-1.58 (m, 2H), 1.58-1.39 (m, 2H), 0.95 (t, J=7.3 Hz, 3H). ³C NMR (101 MHz, CD₂Cl₂) δ 162.67, 150.22, 148.17, 143.94, 142.86, 138.33, 133.95, 1²⁹27, 128.82, 128.71, 128.62, 128.52, 128.28, 128.19, 127.99, 125.98, 117.70, 56.91, 24.72, 21.90, 16.11, 13.42. ESI-MS (m/z): 412.1 [M+H]⁺

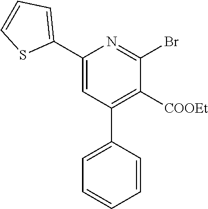 [SMILES: CCOC(=O)c1c(-c2ccccc2)cc(-c2cccs2)nc1Br]

Example 11-1: 2-Bromo-4-phenyl-6-(thiophen-2-yl)nicotinate was prepared in 51% yie^{l}d via cyclization reaction of ethyl 2-cyano-4-oxo-2-phenyl-4-(thiophen-2-yl)butanoate with bromine according to the procedure reported by Girgis. ¹H NMR (400 MHz, CDCl₃) δ 7.67 (d, J=3.7 Hz, 1H), 7.55 (s, 1H), 7.51-7.35 (m, 5H), 7.15-7.10 (m, 1H), 7.07 (d, J=5.1 Hz, 1H), 4.18 (q, J=7.1 Hz, 2H), 1.10 (t, J=7.1 Hz, 3H).

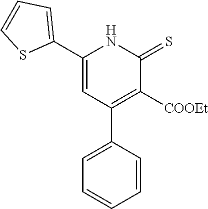 [SMILES: CCOC(=O)c1c(-c2ccccc2)cc(-c2cccs2)[nH]c1=S]

Example 11-2: Ethyl 4-phenyl-6-(thiophen-2-yl)-2-thioxo-1,2-dihydropyridine-3-carboxylate. To the solution of ethyl 2-bromo-4-phenyl-6-(thiophen-2-yl)nicotinate (90 mg, 0.23 mmol) in DMF (300 μL) was added sodium sulfide (36 mg, 0.46 mmol, 2.0 equiv.) and the reaction mixture was stirred at 50° C. The progress of the reaction was followed by LCMS. Once complete, the reaction was diluted with EtOAc and washed with water, dried over magnesium sulfate, filtered and concentrated under reduced pressure. The crude compound was purified by column chromatography to give product in 62% yield. ESI-MS (m/z): 342.1 [M+H]⁺.

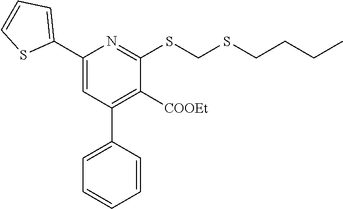 [SMILES: CCCCSCSc1nc(-c2cccs2)cc(-c2ccccc2)c1C(=O)OCC]

Example 11-3: Ethyl 2-(((butylthio)methyl)thio)-4-phenyl-6-(thiophen-2-yl)nicotinate. To the suspension of ethyl 4-phenyl-6-(thiophen-2-yl)-2-thioxo-1,2-dihydropyridine-3-carboxylate (52 mg, 0.15 mmol) in 2 mL of CH₃CN was added Et₃N (45 mg, 0.45 mmol, 3.0 equiv.), and butyl(chloromethyl)sulfane (0.23 mmol, 32 mg). The reaction mixture was stirred at 80° C. for 20 min. Once complete, the reaction was diluted with EtOAc and water. The organic phase was separated and aqueous layer was extracted twice with EtOAc. The combined extractions were washed with saturated NaCl solution, dried over magnesium sulfate, filtered and concentrated under reduced pressure. The residue was purified by flash chromatography to give product (50%). ¹H NMR (400 MHz, CDCl₃) δ 7.64 (dt, J=3.7, 0.9 Hz, 1H), 7.48-7.39 (m, 4H), 7.40-7.34 (m, 3H), 7.12 (ddd, J=5.0, 3.7, 0.7 Hz, 1H), 4.49 (s, 2H), 4.09 (q, J=7.1 Hz, 2H), 2.73 (t, J=7.5 Hz, 2H), 1.71-1.59 (m, 2H), 1.41 (h, J=7.4 Hz, 2H), 0.95 (t, J=7.1 Hz, 3H), 0.90 (t, J=7.4 Hz, 3H). ESI-MS (m/z): 444.0 [M+H]⁺.

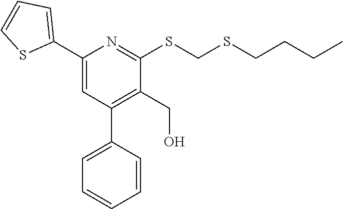 [SMILES: CCCCSCSc1nc(-c2cccs2)cc(-c2ccccc2)c1CO]

Example 11-4: (2-(((butylthio)methyl)thio)-4-phenyl-6-(thiophen-2-yl)pyridin-3-yl)methanol. To the solution of ethyl 2-(((butylthio)methyl)thio)-4-phenyl-6-(thiophen-2-yl)nicotinate (29 mg, 0.065 mmol) in 2.5 mL of THF was added LiAlH₄ (0.072 mmol, 72 μL, 1.1 equiv, 1.0 M solution in THF) at −78° C. The reaction mixture was stirred at −78° C. for 1 h and then was warmed up to room temperature. Once complete, the reaction was diluted with EtOAc and washed with water, dried over magnesium sulfate, filtered and concentrated under reduced pressure to give product in the quantitative yield. ¹H NMR (400 MHz, CDCl₃) δ 7.58 (dd, J=3.7, 1.1 Hz, 1H), 7.54-7.42 (m, 5H), 7.40 (dd, J=5.0, 1.1 Hz, 1H), 7.33 (s, 1H), 7.10 (dd, J=5.1, 3.7 Hz, 1H), 4.61 (s, 2H), 4.58 (s, 2H), 2.73 (t, J=7.6 Hz, 2H), 1.65 (tt, J=8.7, 6.8 Hz, 2H), 1.48-1.32 (m, 2H), 0.90 (t, J=7.4 Hz, 3H). ESI-MS (m/z): 402.1 [M+H]⁺.

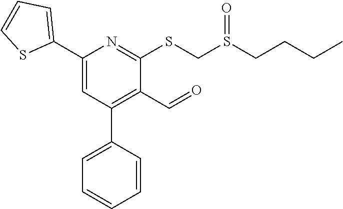 [SMILES: CCCCS(=O)CSc1nc(-c2cccs2)cc(-c2ccccc2)c1C=O]

Example 11-5: 2-(((butylsulfinyl)methyl)thio)-4-phenyl-6-(thiophen-2-yl)nicotinaldehyde. To a solution of (2-(((butylthio)methyl)thio)-4-phenyl-6-(thiophen-2-yl)pyridin-3-yl)methanol (50 mg, 0.125 mmol) in 50 mL of dichloromethane at 0° C. was added Dess-Martin periodinane (79.2 mg, 0.187 mmol, 1.5 equiv) and the reaction mixture was stirred for 3 h. Once complete, the reaction was diluted with ethyl acetate and the mixture of 10% aqueous sodium thiosulfate solution in saturated aqueous sodium bicarbonate solution. The organic phase was separated and aqueous layer was extracted twice with Ethyl acetate. The combined extractions were washed with saturated NaCl solution, dried over magnesium sulfate, filtered and concentrated under reduced pressure. The residue was purified by flash chromatography to give product in 82% isolated yield. ¹H NMR (400 MHz, CDCl₃) δ 9.95 (s, 1H), 7.79 (dd, J=3.8, 1.1 Hz, 1H), 7.56 (dd, J=5.0, 1.1 Hz, 1H), 7.55-7.50 (m, 3H), 7.44 (s, 1H), 7.44-7.39 (m, 2H), 7.17 (dd, J=5.0, 3.8 Hz, 1H), 4.88 (d, J=12.7 Hz, 1H), 4.21 (d, J=12.7 Hz, 1H), 3.16-2.92 (m, 1H), 2.91-2.62 (m, 1H), 1.94-1.76 (m, 2H), 1.57-1.36 (m, 2H), 0.95 (t, J=7.3 Hz, 3H). ESI-MS (m/z): 416.0 [M+H]⁺.

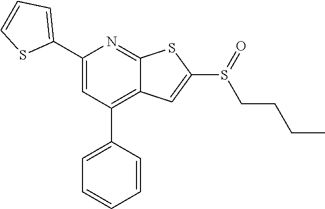 [SMILES: CCCCS(=O)c1cc2c(-c3ccccc3)cc(-c3cccs3)nc2s1]

Example 11: 2-(butylsulfinyl)-4-phenyl-6-(thiophen-2-yl)thieno[2,3-b]pyridine To a solution of 2-(((butylsulfinyl)methyl)thio)-4-phenyl-6-(thiophen-2-yl)nicotinaldehyde (24 mg, 0.058 mmol) in DMF 0.5 mL and 0.25 mL was added aq. solution of KOH (0.089 mmol, 5 mg, 1.5 equiv in 50 μL of water). The reaction mixture was stirred at 32° C. for about 20 min. The reaction was followed by TLC and LCMS. Once complete, the reaction was diluted with EtOAc and washed with water, dried over magnesium sulfate, filtered and concentrated under reduced pressure. The crude compound was purified by flash chromatography to give product in 71% isolated yield. ESI-MS (m/z): ¹H NMR (400 MHz, CD₂Cl₂) δ 7.78 (s, 1H), 7.77 (dd, J=3.7, 1.2 Hz, 1H), 7.73 (s, 1H), 7.70-7.63 (m, 2H), 7.63-7.53 (m, 3H), 7.51 (dd, J=5.1, 1.1 Hz, 1H), 7.18 (dd, J=5.1, 3.7 Hz, 1H), 3.12 (ddd, J=13.1, 8.8, 6.3 Hz, 1H), 3.04 (ddd, J=13.0, 9.0, 6.7 Hz, 1H), 1.85-1.60 (m, 2H), 1.58-1.38 (m, 2H), 0.94 (t, J=7.3 Hz, 3H). ESI-MS (m/z): 398.0 [M+H]⁺.

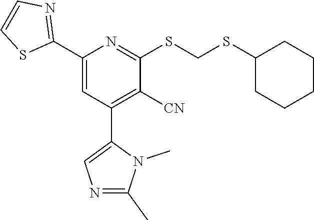 [SMILES: Cc1ncc(-c2cc(-c3nccs3)nc(SCSC3CCCCC3)c2C#N)n1C]

Example 12-1: 2-(((cyclohexylthio)methyl)thio)-4-(1,2-dimethyl-1H-imidazol-5-yl)-6-(thiazol-2-yl)nicotinonitrile was prepared from (E)-3-(1,2-dimethyl-1H-imidazol-5-yl)-1-(thiazol-2-yl)prop-2-en-1-one, 2-cyanoethanethioamide and (chloromethyl)(cyclohexyl)sulfane (3.0 equiv.) in 64% isolated yield, using synthetic procedures described for the preparation of the analog SW209415. ¹H NMR (400 MHz, CDCl₃) δ 7.97 (d, J=3.1 Hz, 1H), 7.87 (s, 1H), 7.57 (d J=3.1, 1H), 7.39 (s, 1H), 4.53 (s, 2H), 3.62 (s, 3H), 2.95 (tt, J=10.4, 3.7 Hz, 1H), 2.49 (s, 3H), 2.13-1.97 (m, 2H), 1.78 (dt, J=12.2, 3.8 Hz, 2H), 1.49-1.19 (m, 6H). EST-MS (m/z): 442.0 [M+H]⁺.

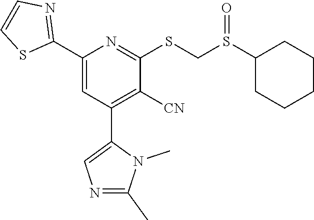 [SMILES: Cc1ncc(-c2cc(-c3nccs3)nc(SCS(=O)C3CCCCC3)c2C#N)n1C]

Example 12-2: 2-(((cyclohexylsulfinyl)methyl)thio)-4-(1,2-dimethyl-1H-imidazol-5-yl)-6-(thiazol-2-yl)nicotinonitrile was prepared via standard oxidation with hydrogen peroxide in 99% isolated yield, using synthetic procedures described for the preparation of the analog SW209415. ¹H NMR (400 MHz, CDCl₃) δ 7.99 (d, J=3.1 Hz, 1H), 7.95 (s, 1H), 7.61 (d, J=3.1 Hz, 1H), 7.44 (s, 1H), 4.62 (d, J=13.3 Hz, 1H), 4.58 (d, J=13.3 Hz, 1H), 3.64 (s, 3H), 2.83 (tt, J=11.9, 3.7 Hz, 1H), 2.52 (s, 3H), 2.08 (s, 3H), 2.04-1.84 (m, 4H), 1.84-1.53 (m, 4H), 1.48-1.32 (m, 2H). ESI-MS (m/z): 458.1 [M+H]⁺.

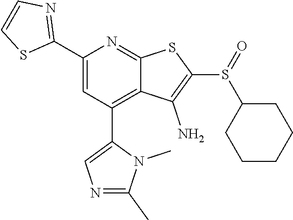 [SMILES: Cc1ncc(-c2cc(-c3nccs3)nc3sc(S(=O)C4CCCCC4)c(N)c23)n1C]

Example 12, 13 and 14: 2-(cyclohexylsulfinyl)-4-(1,2-dimethyl-1H-imidazol-5-yl)-6-(thiazol-2-yl)thieno[2,3-b]pyridin-3-amine was prepared via cyclization reaction with potassium hydroxide in 65% isolated yield, using synthetic procedures described for the preparation of the analog SW209415. ¹H NMR (400 MHz, CD₂Cl₂) δ 8.07 (s, 1H), 7.95 (d, J=3.2 Hz, 1H), 7.56 (d, J=3.2 Hz, 1H), 7.09 (s, 1H), 4.75 (s, 2H), 3.40 (s, 3H), 3.25-3.05 (m, 1H), 2.47 (s, 3H), 2.23 (d, J=12.6 Hz, 1H), 2.00-1.79 (m, 3H), 1.63-1.53 (m, 1H), 1.49-1.25 (m, 5H). ESI-MS (m/z): 458.1 [M+H]⁺. Enantiomers were separated on a 1 cm Chiralpak OD column using 60% iPrOH and 40% Hex. with 10 mL/min flow rate, 500 μL injection (concentration 10 mg/ml) the 1st peak (Example 13) was at 10.8 min and the 2nd peak (Example 14) was at 13.5 min. Optical Rotation: Pea^{k} 1 [□] −65.9 (c=0.1, EtOH), Peak 2 [□] +85.9 (c=0.1, EtOH).

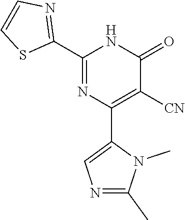 [SMILES: Cc1ncc(-c2nc(-c3nccs3)[nH]c(=O)c2C#N)n1C]

Example 15-1: 4-(1,2-dimethyl-1H-imidazol-5-yl)-6-oxo-2-(thiazol-2-yl)-1,6-dihydropyrimidine-5-carbonitrile. The solution of 1,5-dimethyl-1H-imidazole-2-carbaldehyde (300 mg, 2.42 mmol), ethyl cyanoacetate (273.3 mg, 2.42 mmol) and catalytic amount of pipiridine in MeOH (3 mL) was stirred at room temperature overnight. The solvent was evaporated the crude product of knoevenagel condensation was redissolved in EtOH (4 mL). Thiazole-2-carboximidamide hydrochloride (1.5 equiv, 3.63 mmol, 593 mg) and potassium carbonate (3.0 equiv, 2.25 mmol, 1.0 g) were added and the reaction mixture was stirred at 80° C. overnight. Once completed the reaction mixture was filtered and the solid suspended in water and filtered again to give crude product (260 mg). ESI-MS (m/z): 299.1 [M+H]⁺.

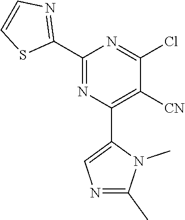 [SMILES: Cc1ncc(-c2nc(-c3nccs3)nc(Cl)c2C#N)n1C]

Example 15-2: 4-chloro-6-(1,2-dimethyl-1H-imidazol-5-yl)-2-(thiazol-2-yl)pyrimidine-5-carbonitrile. The reaction mixture of 4-(1,2-dimethyl-1H-imidazol-5-yl)-6-oxo-2-(thiazol-2-yl)-1,6-dihydropyrimidine-5-carbonitrile (260 mg,) in POCl₃ (300 μL) was stirred at 80° C. for 30 min. Once completed (the reaction was followed by LCMS) the reaction mixture was cooled to −20° C. and a few drops of Et₃N were added followed by EtOAc and water. The organic phase was separated and pH of aqueous layer was adjusted to the 8 to 9 range and extracted twice with EtOAc. The combined extractions were dried over magnesium sulfate, filtered and concentrated under reduced pressure to give desired product (110 mg). ESI-MS (m/z): 317.0 [M+H]⁺.

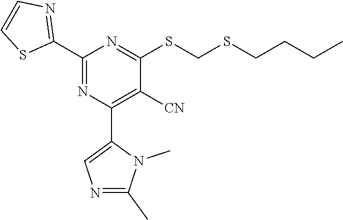 [SMILES: CCCCSCSc1nc(-c2nccs2)nc(-c2cnc(C)n2C)c1C#N]

Example 15-3: 4-(((butylthio)methyl)thio)-6-(1,2-dimethyl-1H-imidazol-5-yl)-2-(thiazol-2-yl)pyrimidine-5-carbonitrile To 4-chloro-6-(1,2-dimethyl-1H-imidazol-5-yl)-2-(thiazol-2-yl)pyrimidine-5-carbonitrile (60 mg, 0.190 mmol) in DMF (200 μL) was added sodium sulfide (22 mg, 0.28 mmol, 1.5 equiv.) and the reaction mixture was stirred at 80° C. for 20 min. The progress of the reaction was followed by LCMS. Once complete, conc. HCl was added and the reaction mixture was stirred in the hood for 30 min. The reaction mixture was concentrated under reduced pressure to give crude 4-(1,2-dimethyl-1H-imidazol-5-yl)-2-(thiazol-2-yl)-6-thioxo-1,6-dihydropyrimidine-5-carbonitrile. ESI-MS (m/z): 417.1 [M+H]⁺.

To the solution of 4-(1,2-dimethyl-1H-imidazol-5-yl)-2-(thiazol-2-yl)-6-thioxo-1,6-dihydropyrimidine-5-carbonitrile in CH₃CN (2 mL) was added Et₃N (58 mg, 0.57 mmol, 3.0 equiv.) followed by butyl(chloromethyl)sulfane (0.38 mmol, 52 mg, 2.0 equiv). The reaction mixture was stirred at 80° C. for 30 min. Once complete, the reaction was diluted with EtOAc and water. The organic phase was separated and aqueous layer was extracted twice with EtOAc. The combined extractions were washed with saturated NaCl solution, dried over magnesium sulfate, filtered and concentrated under reduced pressure. The residue was purified by flash chromatography to give 65 mg of product (83%). ¹H NMR (400 MHz, CD₂Cl₂) δ 8.12 (d, J=3.1 Hz, 1H), 8.09 (s, 1H), 7.71 (d, J=3.1 Hz, 1H), 4.62 (s, 2H), 4.01 (s, 3H), 2.76 (t, J=7.4 Hz, 2H) 2.50 (s, 3H), 1.66 (tt, J=8.2, 6.9 Hz, 2H), 1.42 (h, J=7.5 Hz, 2H) 0.95-0.85 (t, J=7.4 Hz, 3H). ESI-MS (m/z): 417.0 [M+H]⁺.

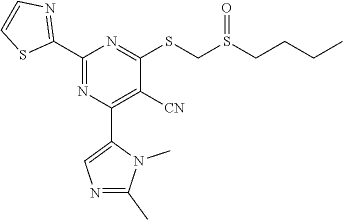 [SMILES: CCCCS(=O)CSc1nc(-c2nccs2)nc(-c2cnc(C)n2C)c1C#N]

Example 15-4: 4-(((butylsulfinyl)methyl)thio)-6-(1,2-dimethyl-1H-imidazol-5-yl)-2-(thiazol-2-yl)pyrimidine-5-carbonitrile was prepared via standard oxidation with hydrogen peroxide in 87% isolated yield, using synthetic procedures described for the preparation of the analog SW209415. ¹H NMR (400 MHz, CD₂Cl₂) δ 8.15 (s, 1H), 8.12 (d, J=3.1 Hz, 1H), 7.74 (d, J=3.1 Hz, 1H), 4.77 (d, J=13.3 Hz, 1H), 4.56 (d, J=13.3 Hz, 1H), 4.01 (s, 3H), 3.06 (dt, J=12.9, 8.4 Hz, 1H), 2.96-2.86 (m, 1H), 2.51 (s, 3H), 1.87-1.74 (m, 2H), 1.59-1.44 (m, 2H), 0.96 (t, J=7.4 Hz, 3H). ESI-MS (m/z): 433.0 [M+H]⁺.

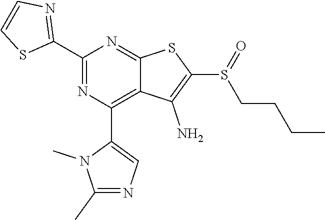 [SMILES: CCCCS(=O)c1sc2nc(-c3nccs3)nc(-c3cnc(C)n3C)c2c1N]

Example 15: 6-(butylsulfinyl)-4-(1,2-dimethyl-1H-imidazol-5-yl)-2-(thiazol-2-yl)thieno[2,3-d]pyrimidin-5-amine To a solution of 4-(((butylsulfinyl)methyl)thio)-6-(1,2-dimethyl-1H-imidazol-5-yl)-2-(thiazol-2-yl)pyrimidine-5-carbonitrile (0.092 mmol, 40 mg) in DMF (1.0 mL) was added KOH (0.046 mmol, 2.6 mg, 0.5 equiv. in 26 al of water). The reaction mixture was stirred at r.t. for 20 min (the reaction was monitored by TLC). Once complete, the reaction was diluted with EtOAc and washed with 5% aq. solution of acetic acid. The organic phase was separated and aqueous layer was extracted twice with EtOAc, dried over magnesium sulfate, filtered and concentrated under reduced pressure to give crude product, which was purified by flash chromatography in 27% isolated yield. ¹H NMR (400 MHz, CD₂Cl₂) δ 8.06 (dd, J=3.1 Hz, 1H), 7.62 (d, J=3.1, 1H), 7.45 (s, 1H), 5.37 (s, 2H), 3.88 (s, 3H), 3.43-3.25 (m, 1H), 3.25-3.01 (m, 1H), 2.53 (s, 3H), 1.92-1.61 (m, 2H), 1.65-1.41 (m, 2H), 0.97 (t, J=7.4 Hz, 3H). ESI-MS (m/z): 433.0 [M+H]⁺.

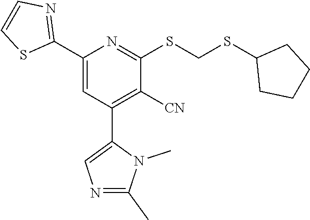 [SMILES: Cc1ncc(-c2cc(-c3nccs3)nc(SCSC3CCCC3)c2C#N)n1C]

Example 16-1: 2-(((cyclopentylthio)methyl)thio)-4-(1,2-dimethyl-1H-imidazol-5-yl)-6-(thiazol-2-yl)nicotinonitrile was prepared from, (E)-3-(1,2-dimethyl-1H-imidazol-5-yl)-1-(thiazol-2-yl)prop-2-en-1-one, 2-cyanoethanethioamide and (chloromethyl)(cyclopentyl)sulfane (3.0 equiv.) in 99% isolated yield, using synthetic procedures described for the preparation of the analog SW209415. ¹H NMR (400 MHz, CDCl₃) δ 7.97 (d, J=3.1 Hz, 1H), 7.86 (s, 1H), 7.57 (d, J=3.1 Hz, 1H), 7.39 (s, 1H), 4.52 (s, 2H), 3.62 (s, 3H), 3.47-3.30 (m, 1H), 2.49 (s, 3H), 2.17-1.93 (m, 2H), 1.87-1.66 (m, 2H), 1.66-1.36 (m, 4H). ESI-MS (m/z): 428.0 [M+H]⁺.

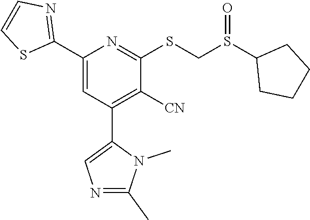 [SMILES: Cc1ncc(-c2cc(-c3nccs3)nc(SCS(=O)C3CCCC3)c2C#N)n1C]

Example 16-2: 2-(((cyclopentylsulfinyl)methyl)thio)-4-(1,2-dimethyl-1H-imidazol-5-yl)-6-(thiazol-2-yl)nicotinonitrile was prepared via standard oxidation hydrogen peroxide in 95% isolated yield, using synthetic procedures described for the preparation of the analog SW209415. ¹H NMR (500 MHz, CDCl₃) δ 8.02 (d, J=3.1 Hz, 1H), 7.98 (s, 1H), 7.64 (d, J=3.1 Hz, 1H), 7.47 (s, 1H), 4.63 (d, J=13.1 Hz, 1H), 4.51 (d, J=13.2 Hz, 1H), 3.67 (s, 3H), 3.40 (tt, J=8.7, 6.9 Hz, 1H), 2.56 (s, 3H), 2.31-1.94 (m, 2H), 1.94-1.53 (m, 6H). EST-MS (m/z): 444.1 [M+H]⁺.

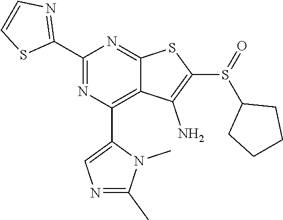 [SMILES: Cc1ncc(-c2nc(-c3nccs3)nc3sc(S(=O)C4CCCC4)c(N)c23)n1C]

Example 16: 2-(cyclopentylsulfinyl)-4-(1,2-dimethyl-1H-imidazol-5-yl)-6-(thiazol-2-yl)thieno[2,3-b]pyridin-3-amine was prepared via cyclization reaction with potassium hydroxide in 50% isolated yield, using synthetic procedures described for the preparation of the analog SW209415. ¹H NMR (400 MHz, CD₂Cl₂) δ 8.07 (d, J=0.9 Hz, 1H), 7.95 (dd, J=3.3, 0.9 Hz, 1H), 7.56 (dd, J=3.2, 0.8 Hz, 1H), 7.09 (s, 1H), 4.76 (s, 2H), 3.87-3.59 (m, 1H), 3.40 (s, 3H), 2.47 (s, 3H), 2.34-2.15 (m, 1H), 2.14-2.02 (m, 1H), 1.93-1.56 (m, 6H). ESI-MS (m/z): 444.1 [M+H]⁺. Enantiomers were separated on a 1 cm Chiralpak OD column using 85% iPrOH and 15% Hex. with 10 mL/min flow rate, 600 μL injection (concentration 20 mg/ml) the 1st peak (Example 18) was at 11.6 min and the 2nd peak (Example 17) was at 15.8 min. Optical Rotation: Peak 1 [□] −61.987 (c=0.1, EtOH), Peak 2 [□] +62.986 (c=0.1, EtOH).

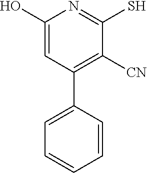 [SMILES: N#Cc1c(-c2ccccc2)cc(O)nc1S]

Example 19-1: 6-hydroxy-2-mercapto-4-phenylnicotinonitrile. To a solution of KOH (12.5 mmol, 699 mg) in EtOH (550 μL) was added dropwise the solution of 2-cyanoethanethioamide (10.4 mmol, 1.05 mg) and ethyl benzoylacetate (10.4 mmol, 2.0 g) in 2.5 mL of MeOH. The reaction mixture was stirred at reflux overnight. After cooling down, the precipitate was collected by filtration and suspended in water, and the mixture was acidified with concentrated HCl, solid separated by filtration and dried under vacuum to yielding 810 mg (34%) of 6-hydroxy-2-mercapto-4-phenylnicotinonitrile. ESI-MS (m/z): 229.0 [M+H]⁺.

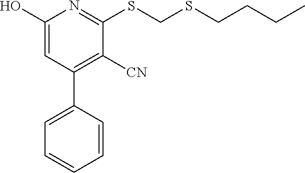 [SMILES: CCCCSCSc1nc(O)cc(-c2ccccc2)c1C#N]

Example 19-2: 2-(((butylthio)methyl)thio)-6-hydroxy-4-phenylnicotinonitrile To a solution of 6-hydroxy-2-mercapto-4-phenylnicotinonitrile (760 mg, 3.33 mmol) was added Et₃N (9.99 mmol, 1.00 mL) followed by butyl(chloromethyl)sulfane (3.0 mmol, 413.5 mg) in CH₃CN (10 mL) and the reaction mixture was stirred at room temperature for 30 min. Once complete, the reaction was diluted with EtOAc and water. The organic phase was separated and aqueous layer was extracted twice with EtOAc. The combined extractions were washed with saturated NaCl solution, dried over magnesium sulfate, filtered and concentrated under reduced pressure. The residue was purified by flash chromatography to give 510 mg of designed product (46%). ¹H NMR (400 MHz, CDCl₃) δ 12.41 (s, 1H), 7.91-7.39 (m, 5H), 6.50 (s, 1H), 4.47 (s, 2H), 2.77 (t, J=7.4 Hz, 2H), 1.79-1.54 (m, 2H), 1.41 (h, J=7.3 Hz, 2H), 0.91 (t, J=7.3 Hz, 3H). ESI-MS (m/z): 331.1 [M+H]⁺.

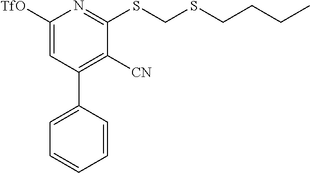 [SMILES: CCCCSCSc1nc(OS(=O)(=O)C(F)(F)F)cc(-c2ccccc2)c1C#N]

Example 19-3: 6-(((butylthio)methyl)thio)-5-cyano-4-phenylpyridin-2-yl trifluoromethanesulfonate To a solution of 2-(((butylthio)methyl)thio)-6-hydroxy-4-phenylnicotinonitrile (0.875 mmol, 289 mg) and t-BuOK (1.05 mmol, 117.6 mg) in 7 mL of THF was added N-Phenyl-bis(trifluoromethanesulfonimide) (1.05 mmol, 375 mmg) under nitrogen. The reaction was stirred for 4 h at room temperature. Once complete, the reaction was diluted with EtOAc and water. The organic phase was separated and aqueous layer was extracted twice with EtOAc. The combined extractions were dried over magnesium sulfate, filtered and concentrated under reduced pressure. The residue was purified by flash chromatography to give 404 mg of designed product (99%). ¹H NMR (400 MHz, CDCl₃) δ 7.76-7.46 (m, 6H), 4.40 (s, 2H), 2.74 (t, J=7.3 Hz, 2H), 1.75-1.56 (m, 2H), 1.51-1.35 (m, 2H), 0.94 (d, J=7.3 Hz, 3H). EST-MS (m/z): 463.0 [M+H]⁺.

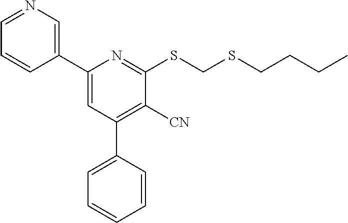 [SMILES: CCCCSCSc1nc(-c2cccnc2)cc(-c2ccccc2)c1C#N]

Example 19-4: 6-(((butylthio)methyl)thio)-4-phenyl-[2,3′-bipyridine]-5-carbonitrile To a solution of 6-(((butylthio)methyl)thio)-5-cyano-4-phenylpyridin-2-yl trifluoromethanesulfonate (40 mg, 0.086 mmol), pyridinylboronic acid (0.129 mmol, 24 mg, 1.5 equiv), [1,1′-bis(diphenylphosphino)ferrocene]dichloropalladium(II) (0.0043 mmol, 3.5 mg, 5 mol %) in degassed THF (200 mL) was added degassed 2M aqueous solution of sodium carbonate (100 mL) and the reaction mixture was stirred for 4 h under nitrogen at 100° C. Once complete, the reaction was diluted with EtOAc and water. The organic phase was separated and aqueous layer was extracted twice with EtOAc, dried over magnesium sulfate, filtered and concentrated under reduced pressure to give crude product, which was purified by flash column chromatography in 30% isolated yield (10 mg). ¹H NMR (400 MHz, CDCl₃) δ 9.31 (d, J=2.1 Hz, 1H), 8.88-8.63 (m, 1H), 8.42 (dt, J=8.0, 2.0 Hz, 1H), 7.68-7.60 (m, 2H), 7.59 (s, 1H), 7.55 (ddd, J=5.5, 3.0, 2.1 Hz, 3H), 7.47 (ddd, J=8.0, 4.8, 1.0 Hz, 1H), 4.54 (s, 2H), 2.76 (t, J=7.3 Hz, 2H), 1.73-1.60 (m, 2H), 1.43 (h, J=7.4 Hz, 2H), 0.92 (t, J=7.4 Hz, 3H). ESI-MS (m/z): 392.1 [M+H]⁺.

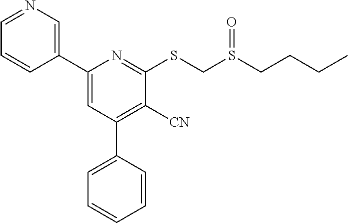 [SMILES: CCCCS(=O)CSc1nc(-c2cccnc2)cc(-c2ccccc2)c1C#N]

Example 19-5: 6-(((butylsulfinyl)methyl)thio)-4-phenyl-[2,3′-bipyridine]-5-carbonitrile prepared via standard oxidation with hydrogen peroxide in 87% isolated yield, using synthetic procedures described for the preparation of the analog SW209415. ¹H NMR (400 MHz, CDCl₃) δ 9.24 (dd, J=2.4, 0.9 Hz, 1H), 8.74 (dd, J=4.8, 1.7 Hz, 1H), 8.49 (ddd, J=8.0, 2.4, 1.6 Hz, 1H), 7.67 (s, 1H), 7.66-7.60 (m, 2H), 7.60-7.54 (m, 3H), 7.51 (ddd, J=8.0, 4.8, 0.9 Hz, 1H), 4.79 (d, J=13.1 Hz, 1H), 4.46 (d, J=13.1 Hz, 1H), 2.96 (dt, J=13.0, 8.1 Hz, 1H), 2.84 (dt, J=13.0, 7.3 Hz, 1H), 1.94-1.77 (m, 2H), 1.59-1.41 (m, 2H), 0.96 (t, J=7.3 Hz, 3H). EST-MS (m/z): 408.1 [M+H]⁺.

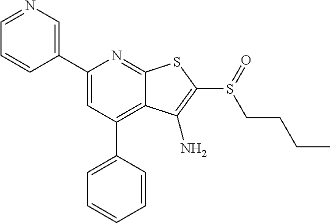 [SMILES: CCCCS(=O)c1sc2nc(-c3cccnc3)cc(-c3ccccc3)c2c1N]

Example 19: 2-(butylsulfinyl)-4-phenyl-6-(pyridin-3-yl)thieno[2,3-b]pyridin-3-amine was prepared via standard cyclization reaction in 81% isolated yield, using synthetic procedures described for the preparation of the analog SW209415. ¹H NMR (400 MHz, CD₂Cl₂) δ 9.30 (s, 1H), 8.67 (d, J=4.7 Hz, 1H), 8.45 (ddd, J=8.1, 2.4, 1.6 Hz, 1H), 7.65 (s, 1H), 7.62-7.50 (m, 5H), 7.45 (dd, J=8.2, 4.6 Hz, 1H), 4.59 (s, 2H), 3.26 (ddd, J=12.8, 9.1, 6.0 Hz, 1H), 3.11 (ddd, J=12.8, 9.1, 6.5 Hz, 1H), 1.82-1.64 (m, 2H), 1.56-1.40 (m, 2H), 0.96 (t, J=7.3 Hz, 3H). ESI-MS (m/z): 408.1 [M+H]⁺.

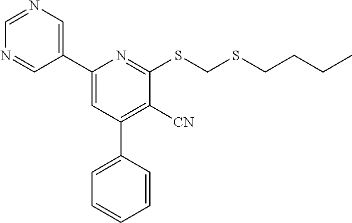 [SMILES: CCCCSCSc1nc(-c2cncnc2)cc(-c2ccccc2)c1C#N]

Example 20-1: 2-(((butylthio)methyl)thio)-4-phenyl-6-(pyrimidin-5-yl)nicotinonitrile was prepared from, 6-(((butylthio)methyl)thio)-5-cyano-4-phenylpyridin-2-yl trifluoromethanesulfonate and 5-pyrimidinylboronic acid via suzuki cross-coupling reaction in 45% isolated yield, using synthetic procedures described for the preparation of the analog SW222807. ¹H NMR (400 MHz, CDCl₃) δ 9.42 (s, 2H), 9.33 (s, 1H), 7.67-7.60 (m, 2H), 7.58 (s, 1H), 7.58-7.54 (m, 3H), 4.51 (s, 2H), 2.76 (t, J=7.3 Hz, 2H), 1.71-1.61 (m, 2H), 1.49-1.37 (m, 2H), 0.92 (t, J=7.3 Hz, 3H). ESI-MS (m/z): 393.1 [M+H]⁺.

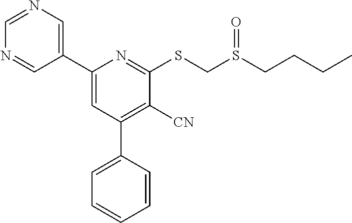 [SMILES: CCCCS(=O)CSc1nc(-c2cncnc2)cc(-c2ccccc2)c1C#N]

Example 20-2: 2-(((butylsulfinyl)methyl)thio)-4-phenyl-6-(pyrimidin-5-yl)nicotinonitrile prepared via standard oxidation with hydrogen peroxide in 95% isolated yield, using synthetic procedures described for the preparation of the analog SW209415. ¹H NMR (400 MHz, CDCl₃) δ 9.41 (s, 2H), 9.35 (s, 1H), 7.65 (s, 1H), 7.64-7.61 (m, 2H), 7.61-7.55 (m, 3H), 4.68 (d, J=13.1 Hz, 1H), 4.56 (d, J=13.2 Hz, 1H), 2.90 (qt, J=12.9, 7.6 Hz, 2H), 1.96-1.77 (m, 2H), 1.64-1.39 (m, 2H), 0.96 (t, J=7.4 Hz, 3H). ESI-MS (m/z): 409.1 [M+H]⁺.

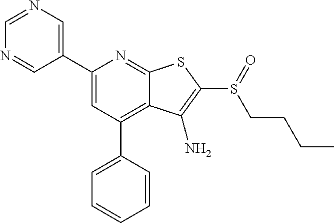 [SMILES: CCCCS(=O)c1sc2nc(-c3cncnc3)cc(-c3ccccc3)c2c1N]

Example 20: 2-(butylsulfinyl)-4-phenyl-6-(pyrimidin-5-yl)thieno[2,3-b]pyridin-3-amine was prepared via standard cyclization reaction of 2-(((butylsulfinyl)methyl)thio)-4-phenyl-6-(pyrimidin-5-yl)nicotinonitrile in 85% isolated yield, using synthetic procedures described for the preparation of the analog SW209415. ¹H NMR (400 MHz, CD₂Cl₂) δ 9.43 (s, 2H), 9.25 (s, 1H), 7.65 (s, 1H), 7.63-7.56 (m, 3H), 7.56-7.49 (m, 2H), 4.61 (s, 2H), 3.27 (ddd, J=12.8, 9.0, 6.1 Hz, 1H), 3.12 (ddd, J=12.8, 9.1, 6.6 Hz, 1H), 1.80-1.64 (m, 2H), 1.55-1.43 (m, 2H), 0.96 (t, J=7.3 Hz, 3H). ESI-MS (m/z): 409.1 [M+H]⁺.

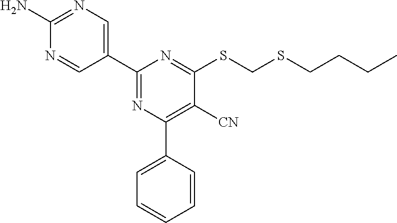 [SMILES: CCCCSCSc1nc(-c2cnc(N)nc2)nc(-c2ccccc2)c1C#N]

Example 21-1: 6-(2-aminopyrimidin-5-yl)-2-(((butylthio)methyl)thio)-4-phenylnicotinonitrile was prepared from, 6-(((butylthio)methyl)thio)-5-cyano-4-phenylpyridin-2-yl trifluoromethanesulfonate and 2-aminopyrimidine-5-boronic acid via suzuki cross-coupling reaction in 34% isolated yield, using synthetic procedures described for the preparation of the analog SW222807. ¹H NMR (400 MHz, CDCl₃) δ 9.00 (s, 2H), 7.61-7.55 (m, 2H), 7.55-7.49 (m, 3H), 7.22 (s, 1H), 5.36 (s, 2H), 4.42 (s, 2H), 2.70 (t, J=7.3 Hz, 2H), 1.68-1.54 (m, 2H), 1.47-1.33 (m, 2H), 0.90 (t, J=7.4 Hz, 3H). ESI-MS (m/z): 408.1 [M+H]⁺.

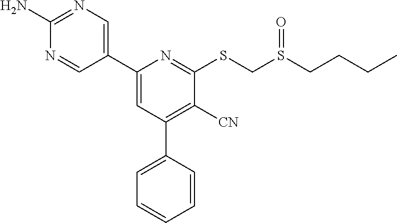 [SMILES: CCCCS(=O)CSc1nc(-c2cnc(N)nc2)cc(-c2ccccc2)c1C#N]

Example 21-2: 6-(2-aminopyrimidin-5-yl)-2-(((butylsulfinyl)methyl)thio)-4-phenylnicotinonitrile prepared via standard oxidation with hydrogen peroxide in 99% isolated yield, using synthetic procedures described for the preparation of the analog SW209415. ¹H NMR (400 MHz, CDCl₃) δ 9.03 (s, 2H), 7.66-7.58 (m, 2H), 7.58-7.49 (m, 3H), 7.47 (s, 1H), 5.55 (s, 2H), 4.73 (d, J=13.1 Hz, 1H), 4.45 (d, J=13.1 Hz, 1H), 2.94 (dt, J=12.9, 8.1 Hz, 1H), 2.83 (dt, J=12.9, 7.3 Hz, 1H), 1.88-1.79 (m, 2H), 1.57-1.41 (m, 2H), 0.96 (t, J=7.4 Hz, 3H). ESI-MS (m/z): 423.8.

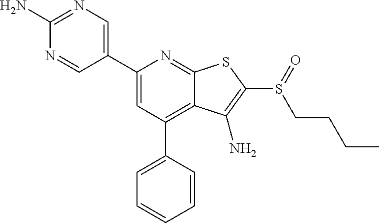 [SMILES: CCCCS(=O)c1sc2nc(-c3cnc(N)nc3)cc(-c3ccccc3)c2c1N]

Example 21: 6-(2-aminopyrimidin-5-yl)-2-(butylsulfinyl)-4-phenylthieno[2,3-b]pyridin-3-amine was prepared via standard cyclization reaction of 6-(2-aminopyrimidin-5-yl)-2-(((butylsulfinyl)methyl)thio)-4-phenylnicotinonitrile in 67% isolated yield, using synthetic procedures described for the preparation of the analog SW209415. ¹H NMR (400 MHz, CD₂Cl₂) δ 9.03 (s, 2H), 7.62-7.54 (m, 3H), 7.54-7.48 (m, 2H), 7.48 (s, 1H), 5.42 (s, 2H), 4.55 (s, 2H), 3.25 (ddd, J=12.8, 9.1, 6.0 Hz, 1H), 3.10 (ddd, J=12.8, 9.2, 6.5 Hz, 1H), 1.82-1.65 (m, 2H), 1.54-1.42 (m, 2H), 0.95 (t, J=7.3 Hz, 3H). ESI-MS (m/z): 424.0.1 [M+H]⁺.

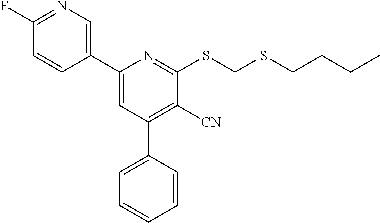 [SMILES: CCCCSCSc1nc(-c2ccc(F)nc2)cc(-c2ccccc2)c1C#N]

Example 22-1: 6-(((butylthio)methyl)thio)-6′-fluoro-4-phenyl-[2,3′-bipyridine]-5-carbonitrile was prepared from, 6-(((butylthio)methyl)thio)-5-cyano-4-phenylpyridin-2-yl trifluoromethanesulfonate and 6-fluoro-3-pyridinylboronic acidvia suzuki cross-coupling reaction in 45% isolated yield, using synthetic procedures described for the preparation of the analog SW222807. ESI-MS (m/z): 410.1 [M+H]⁺.

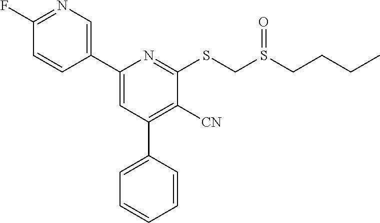 [SMILES: CCCCS(=O)CSc1nc(-c2ccc(F)nc2)cc(-c2ccccc2)c1C#N]

Example 22-2: 6-(((butylsulfinyl)methyl)thio)-6′-fluoro-4-phenyl-[2,3′-bipyridine]-5-carbonitrile prepared via standard oxidation with hydrogen peroxide in 99% isolated yield, using synthetic procedures described for the preparation of the analog SW209415. EST-MS (m/z): 426.1 [M+H]⁺.

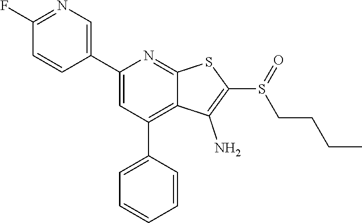 [SMILES: CCCCS(=O)c1sc2nc(-c3ccc(F)nc3)cc(-c3ccccc3)c2c1N]

Example 22: 2-(butylsulfinyl)-6-(6-fluoropyridin-3-yl)-4-phenylthieno[2,3-b]pyridin-3-amine was prepared via standard cyclization reaction of 6-(((butylsulfinyl)methyl)thio)-6′-fluoro-4-phenyl-[2,3′-bipyridine]-5-carbonitrile, using synthetic procedures described for the preparation of the analog SW209415. ¹H NMR (400 MHz, CD₂Cl₂) δ 8.92 (dt, J=2.7, 0.8 Hz, 1H), 8.65-8.50 (m, 1H), 7.61 (s, 1H), 7.60-7.55 (m, 3H), 7.56-7.49 (m, 3H), 7.15-7.04 (m, 1H), 4.59 (s, 2H), 3.26 (ddd, J=12.9, 9.0, 6.0 Hz, 1H), 3.11 (ddd, J=12.8, 9.2, 6.5 Hz, 1H), 1.79-1.63 (m, 2H), 1.55-1.45 (m, 2H), 0.96 (t, J=7.5 Hz, 3H). ESI-MS (m/z): 426.1 [M+H]⁺.

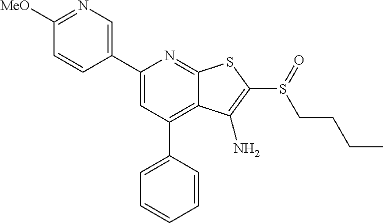 [SMILES: CCCCS(=O)c1sc2nc(-c3ccc(OC)nc3)cc(-c3ccccc3)c2c1N]

Example 23: 2-(butylsulfinyl)-6-(6-methoxypyridin-3-yl)-4-phenylthieno[2,3-b]pyridin-3-amine was the side product of the cyclization reaction of 6-(((butylsulfinyl)methyl)thio)-6′-fluoro-4-phenyl-[2,3′-bipyridine]-5-carbonitrile with potassium hydroxide. ¹H NMR (400 MHz, CD₂Cl₂) δ 8.88 (dd, J=2.5, 0.8 Hz, 1H), 8.38 (dd, J=8.7, 2.5 Hz, 1H), 7.63-7.55 (m, 4H), 7.55-7.48 (m, 2H), 6.87 (dd, J=8.7, 0.8 Hz, 1H), 4.56 (s, 2H), 4.00 (s, 3H), 3.25 (ddd, J=12.7, 9.1, 5.9 Hz, 1H), 3.10 (ddd, J=12.7, 9.2, 6.5 Hz, 1H), 1.79-1.64 (m, 2H), 1.53-1.42 (m, 2H), 0.95 (t, J=7.3 Hz, 3H). ESI-MS (m/z): 438.1 [M+H]⁺.

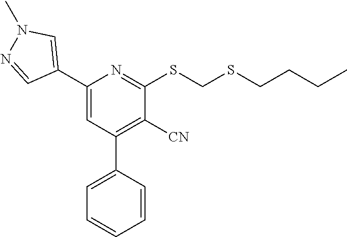 [SMILES: CCCCSCSc1nc(-c2cnn(C)c2)cc(-c2ccccc2)c1C#N]

Example 24-1: 2-(((butylthio)methyl)thio)-6-(1-methyl-1H-pyrazol-4-yl)-4-phenylnicotinonitrile was prepared from 6-(((butylthio)methyl)thio)-5-cyano-4-phenylpyridin-2-yl trifluoromethanesulfonate and (1-methyl-1H-pyrazol-4-yl)boronic acid via suzuki cross-coupling reaction in 89% isolated yield, using synthetic procedures described for the preparation of SW222807. ¹H NMR (400 MHz, CDCl₃) δ 8.02 (s, 1H), 8.00 (s, 1H), 7.63-7.56 (m, 2H), 7.56-7.48 (m, 3H), 7.21 (s, 1H) 4.49 (s, 2H), 3.99 (s, 3H), 2.76 (t, J=7.3 Hz, 2H) 1.68-1.59 (m, 2H), 1.52-1.34 (m, 2H), 0.92 (t, J=7.3 Hz, 3H). ESI-MS (m/z): 394.8.

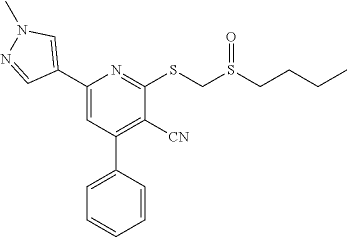 [SMILES: CCCCS(=O)CSc1nc(-c2cnn(C)c2)cc(-c2ccccc2)c1C#N]

Example 24-2: 2-(((butylsulfinyl)methyl)thio)-6-(1-methyl-1H-pyrazol-4-yl)-4-phenylnicotinonitrile prepared via standard oxidation with hydrogen peroxide in 99% isolated yield, using synthetic procedures described for the preparation of SW209415. ¹H NMR (400 MHz, CDCl₃) δ 8.29 (s, 1H), 7.98 (s, 1H), 7.63-7.56 (m, 2H), 7.56-7.47 (m, 3H), 7.31 (s, 1H), 4.84 (d, J=12.9 Hz, 1H), 4.24 (d, J=12.9 Hz, 1H), 3.99 (s, 3H), 2.98 (dt, J=13.0, 8.3 Hz, 1H), 2.80 (ddd, J=13.0, 8.1, 6.9 Hz, 1H), 1.92-1.72 (m, 2H), 1.63-1.41 (m, 2H), 0.96 (t, J=7.4 Hz, 3H). ESI-MS (m/z): 411.1.

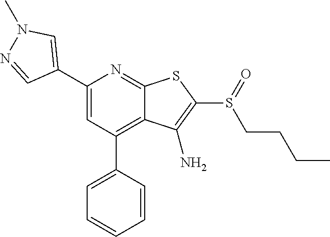 [SMILES: CCCCS(=O)c1sc2nc(-c3cnn(C)c3)cc(-c3ccccc3)c2c1N]

Example 24: 2-(butylsulfinyl)-6-(1-methyl-1H-pyrazol-4-yl)-4-phenylthieno[2,3-b]pyridin-3-amine was prepared via standard cyclization reaction of 2-(((butylsulfinyl)methyl)thio)-6-(1-methyl-1H-pyrazol-4-yl)-4-phenylnicotinonitrile in 70% isolated yield, using synthetic procedures described for the preparation of SW209415. ¹H NMR (400 MHz, CD₂Cl₂) δ 8.03 (s, 1H), 7.99 (s, 1H), 7.62-7.45 (m, 5H), 7.31 (s, 1H), 4.50 (s, 2H), 3.95 (s, 3H), 3.24 (ddd, J=12.8, 9.2, 5.9 Hz, 1H), 3.08 (ddd, J=12.8, 9.3, 6.4 Hz, 1H), 1.86-1.58 (m, 2H), 1.58-1.37 (m, 2H), 0.95 (t, J=7.3 Hz, 3H). ESI-MS (m/z): 411.1 [M+H]⁺.

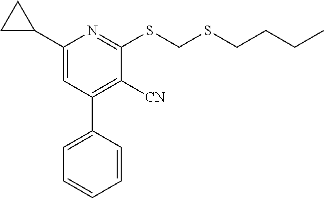 [SMILES: CCCCSCSc1nc(C2CC2)cc(-c2ccccc2)c1C#N]

Example 25-1: 2-(((butylthio)methyl)thio)-6-cyclopropyl-4-phenylnicotinonitrile was prepared from 6-(((butylthio)methyl)thio)-5-cyano-4-phenylpyridin-2-yl trifluoromethanesulfonate and cyclopropylboronic acid via suzuki cross-coupling reaction in 65% isolated yield, using synthetic procedures described for the preparation of SW222807. ESI-MS (m/z): 355.1 [M+H]⁺.

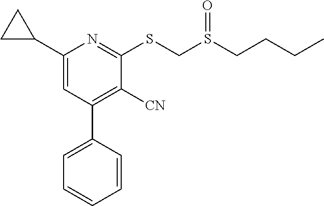 [SMILES: CCCCS(=O)CSc1nc(C2CC2)cc(-c2ccccc2)c1C#N]

Example 25-2: 2-(((butylsulfinyl)methyl)thio)-6-cyclopropyl-4-phenylnicotinonitrile prepared via standard oxidation with hydrogen peroxide in 84% isolated yield, using synthetic procedures described for the preparation of the analog SW209415. ESI-MS (m/z): 471.1.

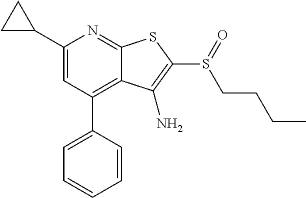 [SMILES: CCCCS(=O)c1sc2nc(C3CC3)cc(-c3ccccc3)c2c1N]

Example 25: 2-(butylsulfinyl)-6-cyclopropyl-4-phenylthieno[2,3-b]pyridin-3-amine was prepared via standard cyclization reaction of 2-(((butylsulfinyl)methyl)thio)-6-cyclopropyl-4-phenylnicotinonitrile in 36% isolated yield, using synthetic procedures described for the preparation of SW209415. ¹H NMR (400 MHz, CD₂Cl₂) δ 7.73-7.31 (m, 5H), 7.01 (s, 1H), 4.47 (s, 2H), 3.21 (ddd, J=12.8, 9.2, 5.9 Hz, 1H), 3.06 (ddd, J=12.7, 9.3, 6.4 Hz, 1H), 2.15 (tt, J=8.1, 4.8 Hz, 1H), 1.80-1.59 (m, 2H), 1.56-1.38 (m, 2H), 1.18-1.12 (m, 2H), 1.12-1.04 (m, 2H), 0.94 (t, J=7.3 Hz, 3H). ESI-MS (m/z): 371.1 [M+H]⁺.

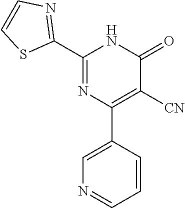 [SMILES: N#Cc1c(-c2cccnc2)nc(-c2nccs2)[nH]c1=O]

Example 26-1: 6-oxo-4-(pyridin-3-yl)-2-(thiazol-2-yl)-1,6-dihydropyrimidine-5-carbonitrile was prepared according to the procedure described for the synthesis of SW222341 in 50% yield, using 3-pyridinecarboxaldehyde as a starting material for the Knoevenagel condensation. EST-MS (m/z): 282.0 [M+H]⁺.

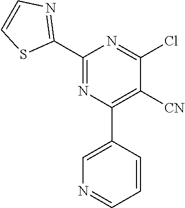 [SMILES: N#Cc1c(Cl)nc(-c2nccs2)nc1-c1cccnc1]

Example 26-2: 4-chloro-6-(pyridin-3-yl)-2-(thiazol-2-yl)pyrimidine-5-carbonitrile was prepared according to the procedure described for the synthesis of SW222341 in 50% yield ESI-MS (m/z): 300.0 [M+H]⁺.

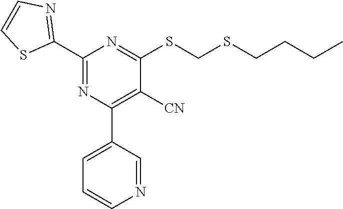 [SMILES: CCCCSCSc1nc(-c2nccs2)nc(-c2cccnc2)c1C#N]

Example 26-3: 4-(((butylthio)methyl)thio)-6-(pyridin-3-yl)-2-(thiazol-2-yl)pyrimidine-5-carbonitrile was was prepared according to the procedure described for the synthesis of SW222341 in 8% yield starting from 6-oxo-4-(pyridin-3-yl)-2-(thiazol-2-yl)-1,6-dihydropyrimidine-5-carbonitrile. ¹H NMR (400 MHz, CD₂Cl₂) δ 9.32 (dd, J=2.4, 0.9 Hz, 1H), 8.83 (dd, J=4.7, 1.6 Hz, 1H), 8.53-8.31 (m, 1H), 8.16 (d, J=3.1 Hz, 1H), 7.70 (d, J=3.1 Hz, 1H), 7.63-7.41 (m, 1H), 4.61 (s, 2H), 2.77 (t, J=7.3 Hz, 2H), 1.75-1.53 (m, 2H), 1.43 (h, J=7.2 Hz, 2H), 0.92 (t, J=7.3 Hz, 3H). ESI-MS (m/z): 400.1 [M+H]⁺.

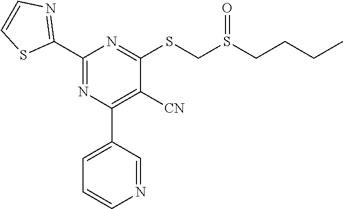 [SMILES: CCCCS(=O)CSc1nc(-c2nccs2)nc(-c2cccnc2)c1C#N]

Example 26-4: 4-(((butylsulfinyl)methyl)thio)-6-(pyridin-3-yl)-2-(thiazol-2-yl)pyrimidine-5-carbonitrile was prepared in 99% isolated yield, via standard oxidation reaction of 4-(((butylthio)methyl)thio)-6-(pyridin-3-yl)-2-(thiazol-2-yl)pyrimidine-5-carbonitrile with hydrogen peroxide, using synthetic procedures described for the preparation of SW222341. ¹H NMR (400 MHz, CDCl₃) 9.45-9.22 (m, 1H), 8.93-8.72 (m, 1H), 8.44 (d, J=8.2 Hz, 1H), 8.16 (d, J=3.2 Hz, 1H), 7.73 (d, J=3.1 Hz, 1H), 7.60-7.43 (m, 1H), 4.78 (d, J=13.3 Hz, 1H), 4.65 (d, J=13.4 Hz, 1H), 3.05 (dt, J=12.9, 8.2 Hz, 1H), 3.00-2.91 (m, 1H), 1.92-1.79 (m, 2H), 1.55-1.42 (m, 2H), 0.97 (t, J=7.3 Hz, 3H). ESI-MS (m/z): 416.1 [M+H]⁺.

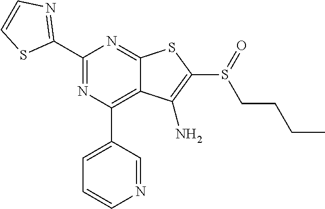 [SMILES: CCCCS(=O)c1sc2nc(-c3nccs3)nc(-c3cccnc3)c2c1N]

Example 26: 6-(butylsulfinyl)-4-(pyridin-3-yl)-2-(thiazol-2-yl)thieno[2,3-d]pyrimidin-5-amine was prepared via cyclization reaction of 4-(((butylsulfinyl)methyl)thio)-6-(pyridin-3-yl)-2-(thiazol-2-yl)pyrimidine-5-carbonitrile in 12% isolated yield, using synthetic procedures described for the preparation of SW222341. ¹H NMR (400 MHz, CD₂Cl₂) δ 8.99 (d, J=2.2 Hz, 1H), 8.78 (dd, J=5.0, 1.7 Hz, 1H), 8.31 (dt, J=7.9, 2.0 Hz, 1H), 8.08 (d, J=3.2 Hz, 1H), 7.89 (d, J=3.1 Hz, 1H), 7.67 (dd, J=7.9, 5.0 Hz, 1H), 3.31 (m, 1H), 3.17 (ddd, J=12.8, 9.3, 6.3 Hz, 1H), 1.80-1.67 (m, 2H), 1.61-1.48 (m, 2H), 0.98 (t, J=7.3 Hz, 3H). ESI-MS (m/z): 416.1 [M+H]⁺.

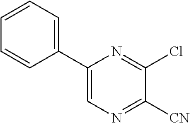 [SMILES: N#Cc1ncc(-c2ccccc2)nc1Cl]

Example 27-1: 3-(((butylthio)methyl)thio)-5-phenylpyrazine-2-carbonitrile. To a solution of 3,5-dichloropyrazine-2-carbonitrile (100 mg, 0.578 mmol), phenylboronic acid (0.635 mmol, 77.5 mg, 1.1 equiv), [1,1′-bis(diphenylphosphino)ferrocene]dichloropalladium(II) (0.029 mmol, 5 mol %) in degassed dioxane/water 4:1 (6 mL) was sodium carbonate (0.635 mmol, 1.1 equiv) and the reaction mixture was stirred for 4 h under nitrogen at 100° C. Once complete, the reaction was diluted with EtOAc and water. The organic phase was separated and aqueous layer was extracted twice with EtOAc, dried over magnesium sulfate, filtered and concentrated under reduced pressure to give product: ESI-MS (m/z): 215.1

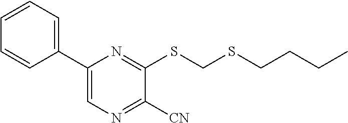 [SMILES: CCCCSCSc1nc(-c2ccccc2)cnc1C#N]

Example 27-2: 3-(((butylthio)methyl)thio)-5-phenylpyrazine-2-carbonitrile To 3-(((butylthio)methyl)thio)-5-phenylpyrazine-2-carbonitrile (100 mg, 0.465 mmol) in DMF (200 μL) was added sodium sulfide (43 mg, 0.558 mmol, 1.2 equiv.) and the reaction mixture was stirred at 80° C. for 20 min. The progress of the reaction was followed by LCMS. Once complete, conc. HCl was added and the reaction mixture was stirred in the hood for 30 min. The reaction mixture was concentrated under reduced pressure to give crude 5-phenyl-3-thioxo-3,4-dihydropyrazine-2-carbonitrile.

To the solution of 5-phenyl-3-thioxo-3,4-dihydropyrazine-2-carbonitrile in CH₃CN (2 mL) was added Et₃N (142 mg, 1.40 mmol, 3.0 equiv.) followed by butyl(chloromethyl)sulfane (0.93 mmol, 128 mg, 2.0 equiv). The reaction mixture was stirred at 80° C. for 30 min. Once complete, the reaction was diluted with EtOAc and water. The organic phase was separated and aqueous layer was extracted twice with EtOAc. The combined extractions were washed with saturated NaCl solution, dried over magnesium sulfate, filtered and concentrated under reduced pressure. The residue was purified by flash chromatography to give 36 mg of product. ¹H NMR (400 MHz, CDCl₃) δ 8.79 (s, 1H), 8.20-7.96 (m, 2H), 7.67-7.45 (m, 3H), 4.50 (s, 2H), 2.73 (t, J=7.6 Hz, 2H), 1.70-1.59 (m, 2H), 1.49-1.35 (m, 2H), 0.91 (t, J=7.4 Hz, 4H). ESI-MS (m/z): 315.1.

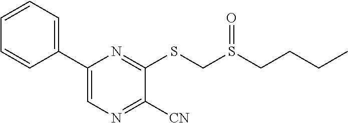 [SMILES: CCCCS(=O)CSc1nc(-c2ccccc2)cnc1C#N]

Example 27-3: 4-(((butylsulfinyl)methyl)thio)-6-(pyridin-3-yl)-2-(thiazol-2-yl)pyrimidine-5-carbonitrile was prepared in 99% isolated yield, via standard oxidation with hydrogen peroxide, using synthetic procedures described for the preparation of SW209415. ¹H NMR (400 MHz, CDCl₃) δ 8.87 (s, 1H), 8.22-7.94 (m, 2H), 7.74-7.43 (m, 3H), 4.70 (d, J=13.2 Hz, 1H), 4.48 (d, J=13.2 Hz, 1H), 3.01-2.75 (m, 2H), 1.89-1.69 (m, 2H), 1.63-1.37 (m, 2H), 0.95 (t, J=7.3 Hz, 4H). ESI-MS (m/z): 331.1

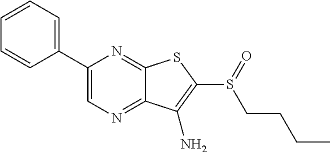 [SMILES: CCCCS(=O)c1sc2nc(-c3ccccc3)cnc2c1N]

Example 27: 6-(butylsulfinyl)-3-phenylthieno[2,3-b]pyrazin-7-amine was prepared from 4-(((butylsulfinyl)methyl)thio)-6-(pyridin-3-yl)-2-(thiazol-2-yl)pyrimidine-5-carbonitrile via standard cyclization reaction of in 90% isolated yield, using synthetic procedures described for the preparation of log SW209415. ¹H NMR (400 MHz, CD₂Cl₂) δ 9.10 (s, 1H), 8.33-7.97 (m, 2H), 7.74-7.35 (m, 3H), 3.34-3.23 (m, 1H), 3.19-3.09 (m, 1H), 1.88-1.69 (m, 2H), 1.58-1.42 (m, 2H), 0.97 (t, J=7.3 Hz, 3H)). ESI-MS (m/z): 331.1

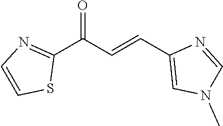

Example 28-1: (E)-3-(1-methyl-1H-imidazol-4-yl)-1-(thiazol-2-yl)prop-2-en-1-one. To a solution of 1-methyl-1H-imidazole-4-carbaldehyde (2.27 mmol, 250 mg) in 6 ml of CH₃CN was added 1-(thiazol-2-yl)-2-(triphenyl-□5-phosphanylidene)ethan-1-one (4.54 mmol, 1.76 g, 2.0 equiv.). The reaction mixture was stirred at 90° C. for 48 h. Once complete, solvent was evaporated and residue was purified by flash chromatography. ¹H NMR (400 MHz, CDCl₃) δ 8.02-7.99 (m, 1H), 7.96 (d, J=15.6 Hz, 1H), 7.88 (d, J=15.7 Hz, 1H), 7.63 (s, 1H), 7.49 (s, 1H), 7.24 (s, 1H). ESI-MS (m/z): 220.0 [M+H]⁺.

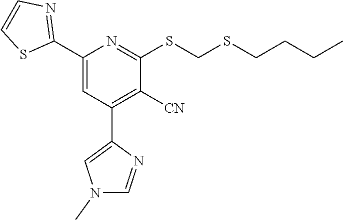 [SMILES: CCCCSCSc1nc(-c2nccs2)cc(-c2cn(C)cn2)c1C#N]

Example 28-2: 2-(((butylthio)methyl)thio)-4-(1-methyl-1H-imidazol-4-yl)-6-(thiazol-2-yl)nicotinonitrile was prepared from (E)-3-(1-methyl-1H-imidazol-4-yl)-1-(thiazol-2-yl)prop-2-en-1-one 2-cyanoethanethioamide and (chloromethyl)(cyclohexyl)sulfane (3.0 equiv.) in 70% isolated yield, using synthetic procedures described for the preparation of SW209415. ¹H NMR (400 MHz, CDCl₃) δ 8.62 (s, 1H), 7.98 (d, J=1.2 Hz, 1H), 7.96 (d, J=3.1 Hz, 1H), 7.57 (d, J=1.3 Hz, 1H), 7.51 (d, J=3.1 Hz, 1H), 4.51 (s, 2H), 3.78 (s, 3H), 2.75 (t, J=7.3 Hz, 2H), 1.77-1.55 (m, 2H), 1.51-1.30 (m, 2H), 0.90 (t, J=7.4 Hz, 3H). ESI-MS (m/z): 402.1 [M+H]⁺.

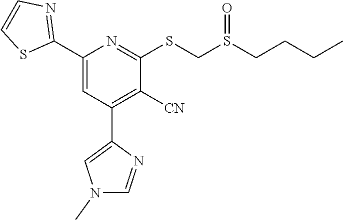 [SMILES: CCCCS(=O)CSc1nc(-c2nccs2)cc(-c2cn(C)cn2)c1C#N]

Example 28-3: 2-(((butylsulfinyl)methyl)thio)-4-(1-methyl-1H-imidazol-4-yl)-6-(thiazol-2-yl)nicotinonitrile was prepared via standard oxidation with hydrogen peroxide in 8% isolated yield, using synthetic procedures described for the preparation of SW209415. ¹H NMR (400 MHz, CDCl₃) δ 8.69 (s, 1H), 8.00 (d, J=1.2 Hz, 1H), 7.99 (d, J=3.2 Hz, 1H), 7.61 (s, 1H), 7.55 (d, J=3.2 Hz, 1H), 4.73 (d, J=13.1 Hz, 1H), 4.43 (d, J=13.1 Hz, 1H), 3.80 (s, 3H), 2.97 (dt, J=12.9, 8.1 Hz, 1H), 2.84 (dt, J=12.9, 7.3 Hz, 1H), 1.91-1.67 (m, 2H), 1.60-1.31 (m, 2H), 0.94 (t, J=7.4 Hz, 3H). ESI-MS (m/z): 418.1 [M+H]⁺.

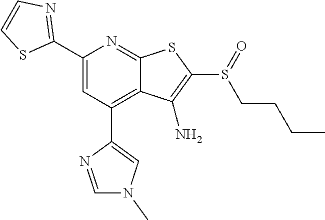 [SMILES: CCCCS(=O)c1sc2nc(-c3nccs3)cc(-c3cn(C)cn3)c2c1N]

Example 28: 2-(butylsulfinyl)-4-(1-methyl-1H-imidazol-4-yl)-6-(thiazol-2-yl)thieno[2,3-b]pyridin-3-amine was prepared from 2-(((butylsulfinyl)methyl)thio)-4-(1-methyl-1H-imidazol-4-yl)-6-(thiazol-2-yl)nicotinonitrile via standard cyclization reaction of in 90% isolated yield, using synthetic procedures described for the preparation of SW209415. ¹H NMR (400 MHz, CD₂Cl₂) ¹H NMR δ 8.25 (s, 1H), 7.94 (d, J=3.3 Hz, 1H), 7.65-7.59 (m, 2H), 7.54 (d, J=3.2, 1H), 7.22 (s, 2H), 3.80 (s, 3H), 3.33-3.19 (m, 1H), 3.19-3.02 (m, 1H), 1.84-1.58 (m, 2H), 1.58-1.37 (m, 2H), 0.95 (t, J=7.4 Hz, 3H). ESI-MS (m/z): 418.1 [M+H]⁺.

### Resolution of Examples 29 and 30 on HPLC

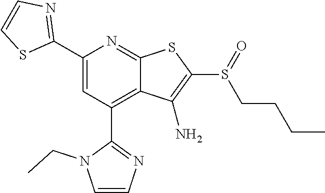 [SMILES: CCCCS(=O)c1sc2nc(-c3nccs3)cc(-c3nccn3CC)c2c1N]

Racemic compound was synthesized analogously to SW209415. Enantiomers were separated on a 1×25 cm Chiralpak AD column using 40% iPrOH and 60% Hex. with 2.5 m/min flow rate, 300 μL injection(concentration 14 mg/ml). The 1st peak (Example 29) was at 17 min and the 2nd peak (Example 30) was at 19 min. Optical Rotation: Peak 1 [□] +92 (c=0.41, EtOH), Peak 2 [□] −6 (c=0.4, EtOH).

### General Procedure A

### Step 1: Aldol Reaction

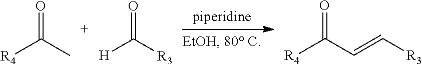

To a solution of the ketone (1.0 equiv) and the aldehyde (2.0 equiv) in ethanol was added piperidine (3.0 equiv). The mixture was stirred at 80° C. for 2 hours. After cooling, the mixture was concentrated and the residue was triturated with a 4:1 mixture of petroleum ether: ethyl acetate. The solid was filtered to give the enone.

Step 2: Condensation with 2-Cyanothioacetamide and Oxidation

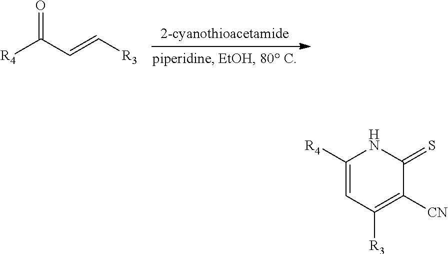

To a solution of the enone (1.0 equiv) in ethanol was added 2-cyanothioacetamide (3.0 equiv) and piperidine (2.0 equiv). The reaction mixture was stirred at 80° C. for 1 hour under an oxygen atmosphere (15 psi). The mixture was concentrated to give the crude pyridinethione.

### Step 3: Preparation of α-Chlorosulfides

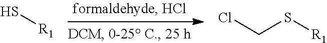

Hydrogen chloride gas was bubbled into a solution of the appropriate thiol (1.0 equiv) and formaldehyde (1.0 equiv) in dichloromethane. The mixture was stirred at 0° C. for 1 hour. The mixture was warmed to 25° C. and stirred for 24 hours. The mixture was concentrated to give the target compound.

### Step 4: Alkylation

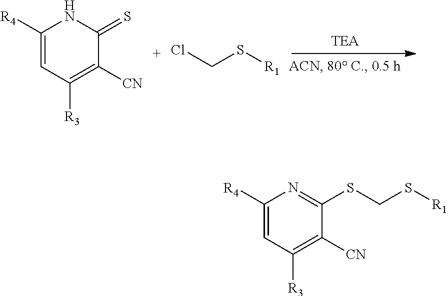

To a solution of the pyridinethione (1.0 equiv) in acetonitrile were added triethylamine (3.0 equiv) and the appropriate chloromethylsulfide (2.0 equiv). The mixture was stirred at 80° C. for 0.5 hour. The mixture was concentrated and the residue was purified by reversed-phase HPLC to give the target compound.

### Step 5 Oxidation to Sulfoxide

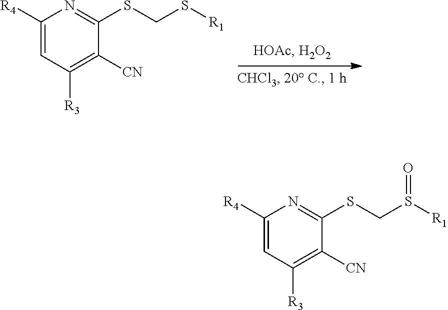

To a solution of the pyridinethiomethylsulfane (1 equiv) in chloroform was added acetic acid (25 equiv) and hydrogen peroxide (30% purity, 1.5 equiv). The mixture was stirred at 20° C. for 1 hour. The mixture was neutralized with saturated sodium bicarbonate solution and extracted with dichloromethane. The combined organic phases were concentrated to give the target sulfoxide.

### Step 6 Cyclization

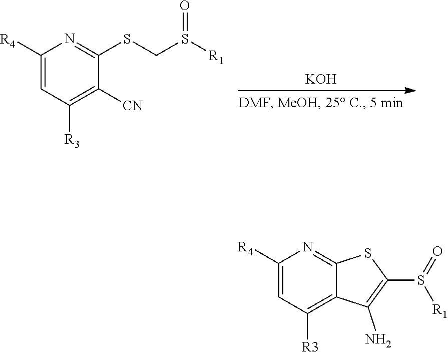

To a solution of the sulfoxide (1 equiv) in methanol and N, N-dimethylformamide was added potassium hydroxide solution (5%, 0.6 equiv). The mixture was stirred at 20° C. for 5 minutes. The mixture was neutralized with aqueous acetic acid (10%) and concentrated. The residue was purified by reversed-phase HPLC to give the final target compound.

The following compounds were prepared from known starting materials using General Procedure A. For select compounds, the more active (+)-R enantiomers were resolved from the racemate using chiral supercritical fluid chromatography (SFC) and are reported with enantiomeric excess (ee) and/or optical rotation values.

| | Structure | | | Chiral |
|---|---|---|---|---|
| Example | of | 400 MHz NMR (CDCl₃ | | compound |
| number | racemate | unless otherwise noted) | LCMS | info |
| 31 | 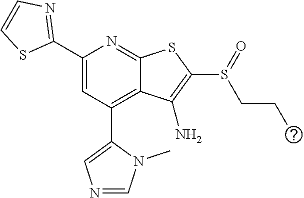 | δ 8.11 (s, 1H), 7.96 (d, J = 3.2 Hz, 1H), 7.70 (s, 1H), 7.55 (d, J = 3.2 Hz, 1H), 4.66 (s, 2H), 3.89-3.91 (m, 1H), 3.76-3.77 (m, 1H), 3.54-3.61 (m, 6H), 1.23 (t, J = 7.2 Hz, 3H). | Exact mass: 433.07, MH+ found: 434.0 | R-isomer: 99% ee; [α] = +7.778° |
| 32 | 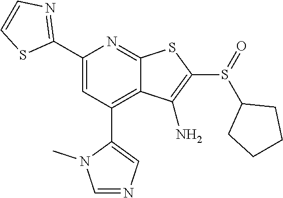 [SMILES: Cn1cncc1-c1cc(-c2nccs2)nc2sc(S(=O)C3CCCC3)c(N)c12] | δ 8.10 (s, 1H), 7.96 (d, J = 3.2 Hz, 1H), 7.69 (s, 1H), 7.54 (d, J = 3.2 Hz, 1H), 7.28 (s, 1H), 4.69 (s, 2H), 3.74- 3.82 (m, 1H), 3.55 (s, 3H), 2.25-2.35 (m, 1H), 2.05- 2.15 (m, 1H), 1.84-1.93 (m, 1H), 1.65-1.82 (m, 5H) | Exact mass: 439.08, MH+ found: 430.1 | R-isomer: 99.99% ee; [α] = +72.987° |
| 33 | 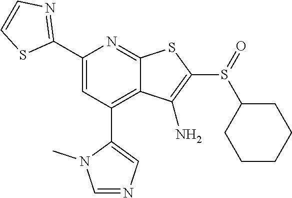 [SMILES: Cn1cncc1-c1cc(-c2nccs2)nc2sc(S(=O)C3CCCCC3)c(N)c12] | δ 8.10 (s, 1H), 7.96 (d, J = 3.2 Hz, 1H), 7.68 (s, 1H), 7.54 (d, J = 3.2 Hz, 1H), 7.28 (s, 1H), 4.69 (s, 2H), 3.55 (s, 3H), 3.15-3.21 (m, 1H), 2.25-2.28 (m, 1H), 1.81-2.00 (m, 3H), 1.72-1.73 (m, 1H), 1.26-1.42 (m, 5H). | Exact mass: 443.09, MH+ found: 444.1 | R-isomer: 99.99% ee; [α]= +88.562° |
| 34 | 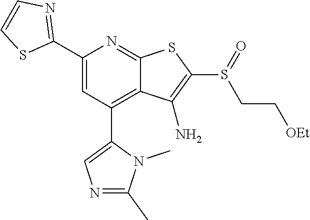 [SMILES: CCOCCS(=O)c1sc2nc(-c3nccs3)cc(-c3cnc(C)n3C)c2c1N] | □□8.08 (s, 1H), 7.96 (d, J = 3.2 Hz, 1H), 7.53 (d, J = 3.2 Hz, 1H), 7.14 (s, 1H), 4.76 (s, 2H), 3.54-3.91 (m, 5H), 3.43 (s, 3H), 3.27-3.29 (m, 1H), 2.51 (s, 3H), 1.23 (t, J = 7.2 Hz, 3H). | Exact mass: 447.09, MH+ found: 448.1 | |
| 35 | 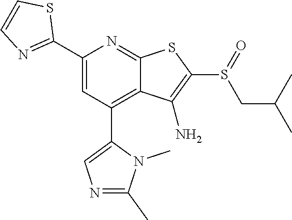 [SMILES: Cc1ncc(-c2cc(-c3nccs3)nc3sc(S(=O)CC(C)C)c(N)c23)n1C] | □□8.08 (s, 1H), 7.95 (d, J = 3.2 Hz, 1H), 7.54 (d, J = 3.2 Hz, 1H), 7.14 (s, 1H), 4.77 (brs, 2H), 3.43 (s, 3H), 3.32- 3.37 (m, 1H), 2.87-2.92 (m, 1H), 2.51 (s, 3H), 3.16-2.23 (m, 1H), 1.14 (d, J = 6.8 Hz, 6H). | Exact mass: 431.09, MH+ found: 432.1 | |
| 36 | 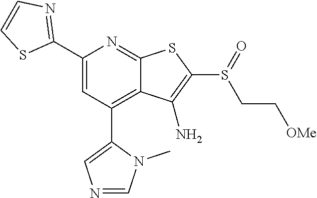 [SMILES: COCCS(=O)c1sc2nc(-c3nccs3)cc(-c3cncn3C)c2c1N] | □□8.11 (s, 1H), 7.96 (d, J = 3.2 Hz, 1H), 7.69 (s, 1H), 7.54 (d, J = 3.2 Hz, 1H), 4.66 (s, 2H), 3.87-3.89 (m, 1H), 3.72-3.73 (m, 1H), 3.56-3.60 (m, 4H), 3.41 (s, 3H), 3.28- 3.29 (m, 1H). | Exact mass: 419.05, MH+ found: 420.1 | R-isomer: 98.0% ee; [α] = +81.943° |
| 37 | 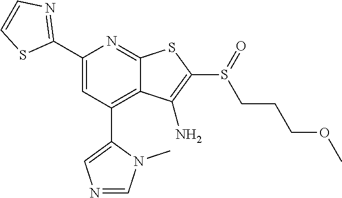 [SMILES: COCCCS(=O)c1sc2nc(-c3nccs3)cc(-c3cncn3C)c2c1N] | δ 8.10 (s, 1H), 7.95 (d, J = 2.8 Hz, 1H), 7.69 (s, 1H), 7.54 (d, J = 2.8 Hz, 1H), 4.69 (s, 2H), 3.55 (s, 3H), 3.52 (t, J = 5.6 Hz, 2H), 3.35- 3.40 (m, 1H), 3.34 (s, 3H), 3.20-3.29 (m, 1H), 1.98- 2.09 (m, 2H). | Exact mass: 433.07, MH+ found: 434.1 | R-isomer: 100.0% ee; [α] = +62.917° |
| 38 | 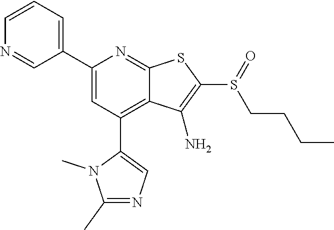 [SMILES: CCCCS(=O)c1sc2nc(-c3cccnc3)cc(-c3cnc(C)n3C)c2c1N] | δ 9.38 (d, J = 2.0 Hz, 1H), 8.72-8.66 (m, 1H), 8.58 (d, J = 8.0 Hz, 1H), 8.00 (s, 1H), 7.56 (dd, J = 8.0 Hz, J = 4.8 Hz, 1H), 7.08 (s, 1H), 5.26 (s, 2H), 3.43 (s, 3H), 3.11- 3.21 (m, 1H), 2.96-3.02 (m, 1H), 2.42 (s, 3H), 1.53- 1.69 (m, 2H), 1.43 (m, 2H), 0.89 (t, J = 7.2 Hz, 3H) | Exact mass: 425.13, MH+ found: 426.2 | R-isomer: 98.44% ee; [α] = +76.953° |
| 39 | 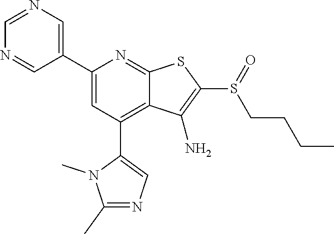 [SMILES: CCCCS(=O)c1sc2nc(-c3cncnc3)cc(-c3cnc(C)n3C)c2c1N] | δ 9.42 (s, 2H), 9.31 (s, 1H), 7.60 (s, 1H), 7.16 (s, 1H), 4.76 (s, 2H), 3.42 (s, 3H), 3.29-3.33 (m, 1H), 3.13- 3.16 (m, 1H), 2.53 (s, 3H), 1.76-1.80 (m, 2H), 1.48-1.54 (m, 2H), 0.97 (t, J = 7.2 Hz, 3H) | Exact mass: 426.13, MH+ found: 427.1 | R-isomer: 98.55% ee; [α] = +75.717° |
| 40 | 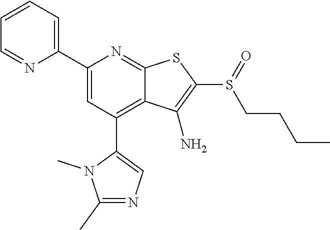 [SMILES: CCCCS(=O)c1sc2nc(-c3ccccn3)cc(-c3cnc(C)n3C)c2c1N] | δ 8.68 (d, J = 4.8 Hz, 1H), 8.55 (d, J = 8.0 Hz, 1H), 8.33 (s, 1H), 7.85-7.89 (m, 1H), 7.35-7.38 (m, 1H), 7.13 (s, 1H), 4.77 (s, 2H), 3.42 (s, 3H), 3.28-3.35 (m, 1H), 3.10-3.17 (m, 1H), 2.50 (s, 3H), 1.72-1.78 (m, 2H), 1.45-1.58 (m, 2H), 0.96 t, J = 7.2 Hz, 3H) | Exact mass: 425.13, MH+ found: 426.3 | |
| 41 | 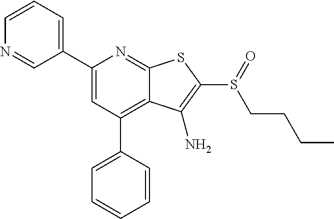 [SMILES: CCCCS(=O)c1sc2nc(-c3cccnc3)cc(-c3ccccc3)c2c1N] | □□9.29 (d, J = 1.2 Hz, 1H), 8.69 (d, J = 3.2 Hz, 1H), 8.45 (dd, J₁ = 6 Hz, 1H, J₂ = 2 Hz, 1H), 7.60 (s, 1H), 7.57 (d, J = 2.4 Hz, 1H), 7.44-7.46 (m, 3H), 4.64 (s, 2H), 3.28-3.33 (m, 1H), 3.14-3.17 (m, 1H), 1.72-1.76 (m, 2H), 1.50-1.52 (m, 2H), 0.96 (t, J = 7.2 Hz, 3H). | Exact mass: 407.11, MH+ found: 408.1 | R-isomer: 100.0% ee; [α] = +81.842° |
| 42 | 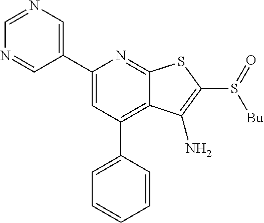 | □□9.44 (s, 2H), 9.30 (s, 1H), 7.52-7.60 (m, 6H), 4.66 (m, 1H), 3.29-3.32 (m, 1H), 3.14-3.18 (m, 1H), 1.75-1.77 (m, 2H), 1.50-1.55 (m, 2H), 0.97 (t, J = 7.2 Hz, 3H). | Exact mass: 408.11, MH+ found: 409.0 | R-isomer: 100.0% ee; [α] = +84.077° |
| 43 | 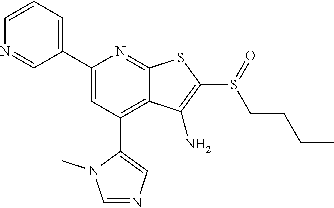 [SMILES: CCCCS(=O)c1sc2nc(-c3cccnc3)cc(-c3cncn3C)c2c1N] | 9.27 (d, J = 2.0 Hz, 1H), 8.69 (d, J = 4.8 Hz, 1H), 8.43 (d, J = 8.0 Hz, 1H), 7.69 (s, 1H), 7.62 (s, 1H), 7.43-7.46 (m, 1H), 7.25 (s, 1H), 4.62 (s, 2H), 3.53 (s, 3H), 3.25-3.32 (m, 1H), 3.08-3.15 (m, 1H), 1.69-1.79 (m, 2H), 1.43-1.55 (m, 2H), 0.95 (t, J = 7.2 Hz, 3H) | Exact mass: 411.12, MH+ found: 412.2 | R-isomer: 99.0% ee; [α] = +63.940° |
| 44 | 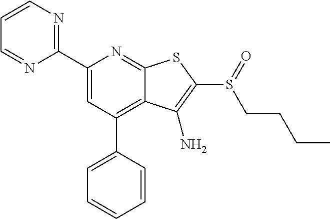 [SMILES: CCCCS(=O)c1sc2nc(-c3ncccn3)cc(-c3ccccc3)c2c1N] | δ 8.96 (d, J = 4.8 Hz, 2H), 8.43 (s, 1H), 7.55 (s, 5H), 7.35 (d, J = 4.8 Hz, 1H), 4.69 (s, 2H), 3.27-3.38 (m, 1H), 3.13-3.17 (m, 1H), 1.68- 1.83 (m, 2H), 1.43-1.57 (m, 2H), 0.97-0.93 (t, J = 7.2 Hz, 3H) | Exact mass: 408.11, MH+ found: 409.1 | |
| 45 | 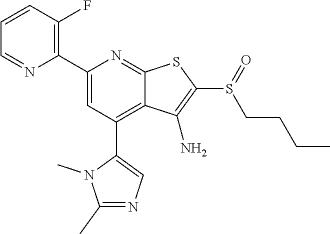 [SMILES: CCCCS(=O)c1sc2nc(-c3ncccc3F)cc(-c3cnc(C)n3C)c2c1N] | 8.63-8.65 (m, 1H), 7.90 (d, J = 1.2 Hz, 1H), 7.60-7.65 (m, 1H), 7.43-7.45 (m, 1H), 7.17 (s, 1H), 4.82 (s, 2H), 3.44 (s, 3H), 3.29-3.36 (m, 1H), 3.13-3.20 (m, 1H), 2.53 (s, 3H), 1.73-1.83 (m, 2H), 1.48-1.58 (m, 2H), 0.98 (t, J = 7.2 Hz, 3H). | Exact mass: 443.12, MH+ found: 444.3 | |
| 46 | 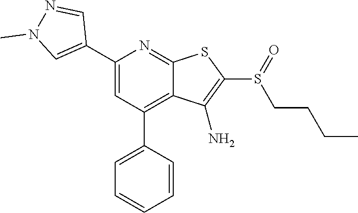 [SMILES: CCCCS(=O)c1sc2nc(-c3cnn(C)c3)cc(-c3ccccc3)c2c1N] | δ 8.05 (s, 1H), 8.01 (s, 1H), 7.54 (d, J = 4.8, 3H), 7.48 (br s, 2H), 7.27 (s, J = 1.6, 1H), 4.54 (s, 2H), 3.98 (s, 3H), 3.26-3.33 (m, 1H), 3.08-3.15 (m, 1H), 1.67-1.79 (m, 2H), 1.46-1.53 (m, 2H), 0.95 (d, J = 7.2 Hz, 3H). | Exact mass: 410.12, MH+ found: 411.3 | R-isomer: 99.0% ee; [α] = +44.752° |
| 47 | 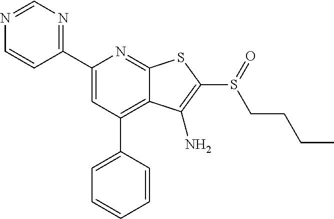 [SMILES: CCCCS(=O)c1sc2nc(-c3ccncn3)cc(-c3ccccc3)c2c1N] | δ 9.29 (d, J = 1.2 Hz, 1H), 8.92 (d, J = 5.2 Hz, 1H), 8.51 (d, J = 4.0 Hz, 1H), 8.38 (s, 1H), 7.62-7.47 (m, 5H), 4.69 (s, 2H), 3.39-3.26 (m, 1H), 3.14 (m, 1H), 1.84- 1.70 (m, 2H), 1.58-1.44 (m, 2H), 0.98-0.94 (t, J = 7.2 Hz, 3H) | Exact mass: 408.11, MH+ found: 409.3 | R-isomer: 96.96% ee; [α] = +57.151° |
| 48 | 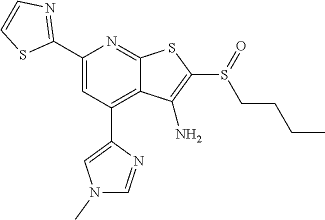 [SMILES: CCCCS(=O)c1sc2nc(-c3nccs3)cc(-c3cn(C)cn3)c2c1N] | δ 8.21 (s, 1H), 7.94 (d, J = 3.6 Hz, 1H), 7.60-7.55 (m, 2H), 7.50 (d, J = 2.8 Hz, 1H), 7.16 (s, 2H), 3.81 (s, 3H), 3.31-3.27 (m, 1H), 3.18-3.14 (m, 1H), 1.79- 1.71 (m, 1H), 1.70-1.62 (m, 1H), 1.50-1.47 (m, 2H), 0.94 (t, J = 7.2 Hz, 3H) | Exact mass: 417.08, MH+ found: 418.1 | R-isomer: 99.0% ee; [α] = +37.834° |
| 49 | 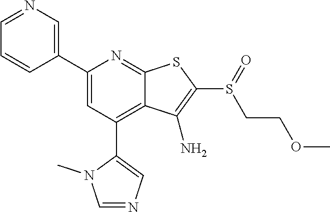 [SMILES: COCCS(=O)c1sc2nc(-c3cccnc3)cc(-c3cncn3C)c2c1N] | δ 9.29 (s, 1H), 8.73-8.71 (m, 1H), 8.47-8.45 (m, 1H), 7.71 (s, 1H), 7.64 (s, 1H), 7.48- 7.45 (m, 1H), 7.29 (s, 1H), 4.60 (s, 2H), 3.89-3.87 (m, 1H), 3.73-3.72 (m, 1H), 3.72-3.70 (m, 1H), 3.55 (s, 3H), 3.41 (s, 3H), 3.30-3.28 (m, 1H) | Exact mass: 413.10, MH+ found: 414.1 | |
| 50 | 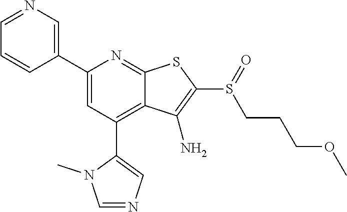 [SMILES: COCCCS(=O)c1sc2nc(-c3cccnc3)cc(-c3cncn3C)c2c1N] | δ 9.29 (s, 1H), 8.72-8.71 (m, 1H), 8.46-8.44 (m, 1H), 7.71 (s, 1H), 7.64 (s, 1H), 7.48-7.44 (m, 1H), 7.29 (s, 1H), 4.64 (s, 2H), 3.55 (s, 3H), 3.52 (t, J = 6.0 Hz, 2H), 3.40-3.33 (m, 4H), 3.28- 3.26 (m, 1H), 2.09-1.99 (m, 2H). | Exact mass: 427.11, MH+ found: 428.1 | R-isomer: 100.0% ee; [α] = +68.867° |
| 51 | 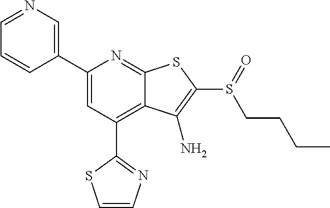 [SMILES: CCCCS(=O)c1sc2nc(-c3cccnc3)cc(-c3nccs3)c2c1N] | □□9.32 (s, 1H), 8.73 (d, J = 4.8 Hz, 1H), 8.48 (d, J = 8.0 Hz, 1H), 8.07 (d, J = 3.2 Hz, 1H), 8.13 (s, 1H), 7.65 (d, J = 3.2 Hz, 1H), 7.47-7.50 (m, 1H), 6.71 (2, 1H), 3.30-3.34 (m, 1H), 3.16-3.19 (m, 1H), 1.73-1.82 (m, 2H), 1.50-1.54 (m, 2H), 0.96 (t, J = 7.2 Hz, 3H). | Exact mass: 414.06, MH+ found: 415.1 | |
| 52 | 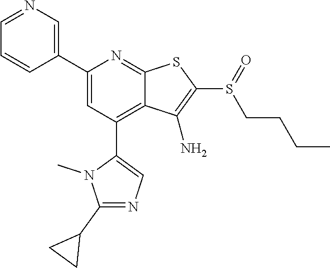 [SMILES: CCCCS(=O)c1sc2nc(-c3cccnc3)cc(-c3cnc(C4CC4)n3C)c2c1N] | □□9.30 (d, J = 2.4 Hz, 1H), 8.72 (d, J = 3.2 Hz, 1H), 8.45-8.48 (m, 1H), 7.64 (s, 1H), 7.46-7.49 (m, 2H), 7.12 (s, 1H), 4.75 (s, 2H), 3.55 (s, 3H), 3.15-3.36 (m, 2 H), 1.52-1.91 (m, 5H), 1.08-1.10 (m, 4H), 0.99 (t, J = 7.2 Hz, 3 H). | Exact mass: 451.15, MH+ found: 452.2 | R-isomer: 99.0% ee; [α] = +68.749° |
| 53 | 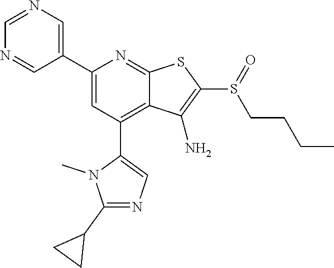 [SMILES: CCCCS(=O)c1sc2nc(-c3cncnc3)cc(-c3cnc(C4CC4)n3C)c2c1N] | δ 9.44 (s, 2H), 9.31 (s, 1H), 7.61 (s, 1H), 7.12 (s, 1H), 4.76 (s, 2H), 3.53 (s, 3H), 3.3- 3.29 (m, 1H), 3.17-3.13 (m, 1H), 1.90-1.821 (m, 1H), 1.79-1.77 (m, 2H), 1.61- 1.51 (m, 2H), 1.10-1.07 (m, 4H), 0.98 (t, J = 7.2 Hz, 3H) | Exact mass: 452.15, MH+ found: 453.2 | |
| 54 | 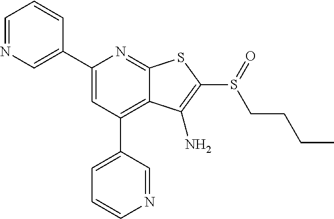 [SMILES: CCCCS(=O)c1sc2nc(-c3cccnc3)cc(-c3cccnc3)c2c1N] | δ 9.30 (d, J = 2.0 Hz, 1H), 8.83 (dd, J = 1.6, 4.8 Hz, 2H), 8.71 (dd, J = 1.6, 4.8 Hz, 1H), 8.45 (td, J = 2.0, 8.4 Hz, 1H), 7.90-7.88 (m, 1H), 7.60 (s, 1H), 7.54-7.50 (m, 1H), 7.47-7.44 (m, 1H), 4.55 (s, 2H), 3.35-3.28 (m, 1H), 3.20-3.10 (m, 1H), 1.84-1.71 (m, 2H), 1.58- 1.44 (m, 2H), 0.97 (t, J = 7.2 Hz, 3H) | Exact mass: 408.11, MH+ found: 409.1 | R-isomer: 99.99% ee; [α] = +44.868° |
| 55 | 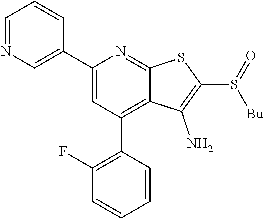 | δ 9.29 (s, 1H), 8.71 (dd, J₁ = 4.8 Hz, J₂ = 1.6 Hz, 1H), 8.47 (dd, J₁ = 4.0 Hz, J₂ = 2.0 Hz, 1H), 7.64 (s, 1H), 7.43- 7.46 (m, 3H), 7.30-7.37 (m, 2H), 4.63 (s, 1H), 4.51 (s, 1H), 3.28-3.31 (m, 1H), 3.12-3.17 (m, 1H), 1.75- 1.79 (m, 2H), 1.62 (s, 2H), 1.50-1.53 (m, 2H), 0.97 (t, J = 7.2 Hz, 3H) | Exact mass: 425.10, MH+ found: 426.2 | |
| 56 | 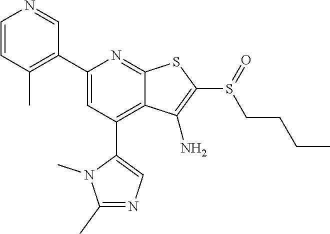 [SMILES: CCCCS(=O)c1sc2nc(-c3cnccc3C)cc(-c3cnc(C)n3C)c2c1N] | δ 8.69 (s, 1H), 8.54 (d, J = 5.2 Hz, 1H), 7.30 (s, 1H), 7.26 (s, 1H), 7.15 (s, 1H), 4.78 (s, 2H), 3.44 (s, 3H), 3.34-3.27 (m, 1H), 3.10- 3.17 (m, 1H), 2.52 (s, 3H), 2.50 (s, 3H), 1.75-1.82 (m, 2H), 1.49-1.56 (m, 2H), 0.98 (s, J = 7.2 Hz, 3H) | Exact mass: 439.15, MH+ found: 440.3 | |
| 57 | 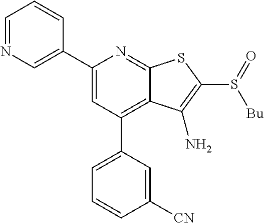 | □□9.29 (d, J = 2.0 Hz, 1H), 8.71 (d, J = 2.0 Hz, J = 4.8 Hz, 1H), 8.45 (d, J = 4.8 Hz, 1H), 7.71-7.88 (m, 4H), 4.50 (d, J = 29.6 Hz, 1H), 3.15- 3.16 (m, 2H), 1.76-1.80 (m, 2H), 1.49-1.54 (m, 2H), 0.98 (t, J = 7.2 Hz, 1H). | Exact mass: 432.11, MH+ found: 433.2 | |
| 58 | 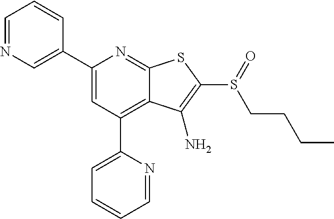 [SMILES: CCCCS(=O)c1sc2nc(-c3cccnc3)cc(-c3ccccn3)c2c1N] | □□9.31 (s, 1H), 8.80 (d, J = 4.4 Hz, 1H), 8.71 (dd, J = 4.4 Hz, J = 1.6 Hz, 1H), 8.49 (d, J = 8.4 Hz, 1H), 7.99 (d, J = 1.6 Hz, 1H), 7.76-7.79 (m, 2H), 7.46-7.48 (m, 2H), 5.71 (s, 2H), 3.14-3.32 (m, 2H), 1.44-1.76 (m, 4H), 0.96 (t, J = 7.2 Hz, 3H). | Exact mass: 408.11, MH+ found: 409.2 | R-isomer: 99.0% ee; [α] = +87.923° |
| 59 | 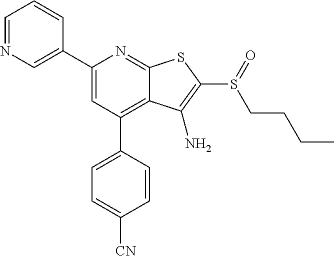 [SMILES: CCCCS(=O)c1sc2nc(-c3cccnc3)cc(-c3ccc(C#N)cc3)c2c1N] | δ 9.22 (d, J = 2.0 Hz, 1H), 8.64 (dd, J₁ = 1.6 Hz, J₂ = 1.6 Hz 4.8 Hz, 1H), 8.37-8.40 (m, 1H), 7.81 (d, J = 8.4 Hz, 2H), 7.61 (d, J = 7.6 Hz, 2H), 7.50 (s, 1H), 7.39 (dd, J₁ = 4.8 Hz, J₂ = 7.6 Hz, 1H), 4.47 (s, 2H), 3.21-3.28 (m, 1H), 3.04-3.12 (m, 1H), 1.67-1.75 (m, 2H), 1.42-1.47 (m, 2H), 0.91 (t, J = 7.2 Hz, 3H). | Exact mass: 432.11, MH+ found: 433.1 | |
| 89 | 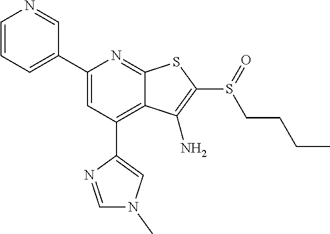 | ¹H NMR: EW8874-453- P1B1, 400 MHz, CDCl₃. δ 9.25 (d, J = 1.6 Hz, 1H), 8.69 (dd, J₁ = 2.0 Hz, J₂ = 4.8 Hz, 1H), 8.46-8.48 (m, 1H), 7.74 (s, 1H), 7.63 (s, 1H), 7.43-7.47 (m, 2H), 6.99 (s, 2H), 3.85 (s, 3H), 3.50- 3.53 (m, 2H), 3.37-3.41 (m, 1H), 3.34 (s, 3H), 3.20-3.26 (m, 1H), 1.98-2.06 (m, 2H). | Exact mass: 428.12 (M + H)⁺ found: 428.2 | R-isomer: 96.5% ee: [a] = +104.018° |

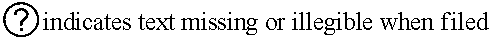

### General Procedure B

### Step 1: Condensation to Form Hydroxypyridine

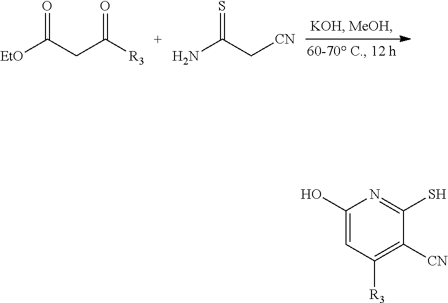

To a solution of potassium hydroxide (1.2 eq) in methanol at 60° C. was added a methanol solution of the R-keto ester (1.0 eq) and 2-cyanothioacetamide (1.0 eq). The reaction was stirred at 70° C. for 12 hours. The mixture was concentrated, dissolved in water and acidified to pH 4 with concentrated hydrochloric acid. The solid was filtered to give the target compound.

### Step 2: Alkylation

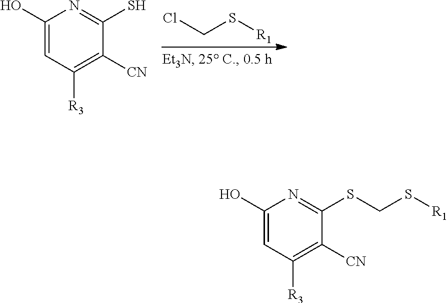

To a solution of the hydroxypyridine (1.0 eq) in acetonitrile were added triethylamine (2.0 eq) and the appropriate chloromethylsulfide (2.0 eq). The mixture was stirred at 25° C. for 15 minutes. The mixture was concentrated and the crude product was purified by prep-HPLC to give the target compound.

### Step 3: Triflation

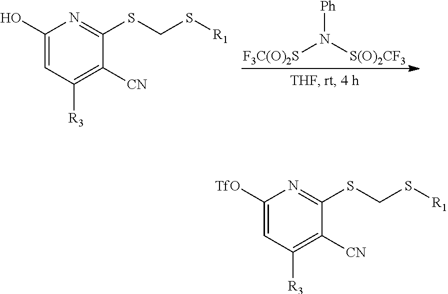

To a solution of the alkylated hydroxypyridine (1.0 eq) in tetrahydrofuran was added potassium tert-butoxide (1.2 eq) and 1,1,1-trifluoro-N-phenyl-N-(trifluoromethylsulfonyl)methanesulfonamide (1.2 eq). The mixture was stirred at 25° C. for 3 hours and concentrated. Water was added and the mixture extracted with dichloromethane. The combined organic layers were concentrated. The residue was purified by column chromatography to give the target compound.

### Step 4: Suzuki Reaction

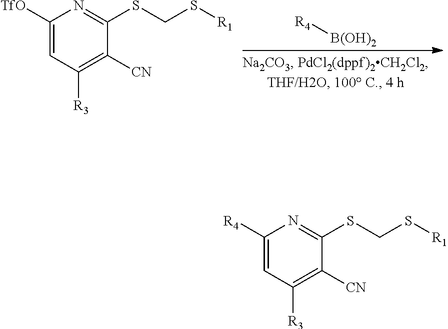

To a solution of the triflate (1.0 eq) in tetrahydrofuran and water was added sodium carbonate (2.0 eq). 1,1′-Bis(diphenylphosphino)ferrocene-palladium(II) dichloride dichloromethane complex (0.05 eq) and the appropriate boronic acid (1.5 eq) were added. The reaction was stirred at 100° C. for 3 hours under nitrogen atmosphere. The mixture was concentrated and water was added. The mixture was extracted with dichloromethane and the combined organic phases were concentrated. The residue was purified by column chromatography to provide the target compound.

### Step 5: Oxidation to Sulfoxide

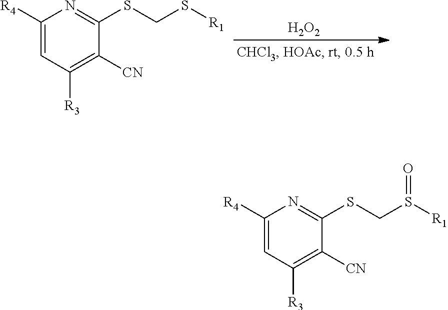

The sulfide was converted to the sulfoxide using the same procedure described in General Procedure A.

### Step 6: Cyclization

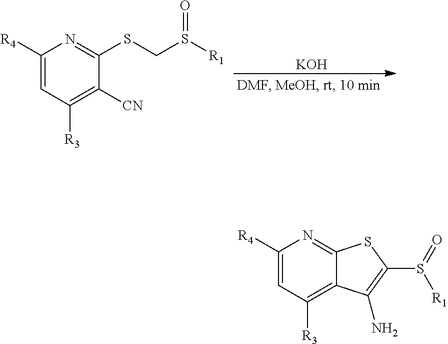

The sulfoxide was cyclized to the final target compound using the same procedure described in General Procedure A.

The following compounds were prepared from known starting materials using General Procedure B. For select compounds, the more active (+)-R enantiomers were resolved from the racemate using chiral supercritical fluid chromatography (SFC) and are reported with ee and/or optical rotation values.

| Example | | 400 MHz NMR (CDCl₃ | | Chiral compound |
|---|---|---|---|---|
| number | Structure | unless otherwise noted) | LCMS | info |
| 60 | 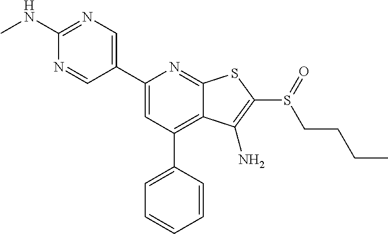 [SMILES: CCCCS(=O)c1sc2nc(-c3cnc(NC)nc3)cc(-c3ccccc3)c2c1N] | δ 9.03 (s, 2H), 7.60-7.44 (m, 5H), 7.40 (s, 1H), 5.40-5.38 (m, 1H), 4.58 (s, 2H), 3.30 (m, 1H), 3.17-3.11 (m, 1H), 3.10 (d, J = 4.8 Hz, 3H), 1.80- 1.66 (m, 2H), 1.56-1.42 (m, 2H), 0.97-0.93 (t, J = 7.4 Hz, 3H) | Exact mass: 437.13, found: 438.2 | R-isomer: 100.0% ee; [α] = +29.707 ° |
| 61 | 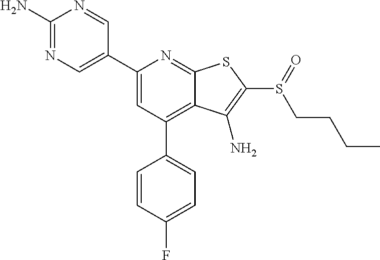 [SMILES: CCCCS(=O)c1sc2nc(-c3cnc(N)nc3)cc(-c3ccc(F)cc3)c2c1N] | δ 9.07 (s, 2H), 7.78 (s, 1H), 7.62-7.66 (m, 2H), 7.44 (t, J = 8.8 Hz, 2H), 7.18 (s, 1H), 4.93 (s, 2H), 3.10-3.15 (m, 1H), 2.99-3.01 (m, 1H), 1.57- 1.62 (m, 1H), 1.40-1.45 (m, 1H), 0.89 (t, J = 7.2 Hz, 3H). | Exact mass: 441.11, found: 442.1 | R-isomer: 99.0% ee; [α] = +87.671° |
| 62 | 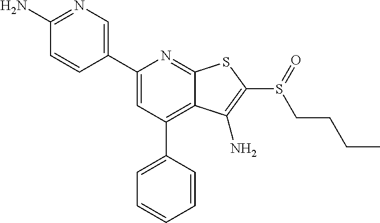 [SMILES: CCCCS(=O)c1sc2nc(-c3ccc(N)nc3)cc(-c3ccccc3)c2c1N] | δ 8.78 (d, J = 2.0 Hz, 1H), 8.27 (dd, J = 2.0, 8.4 Hz, 1H), 7.58-7.47 (m, 5H), 7.47 (s, 1H), 6.62 (d, J = 8.4 Hz, 1H), 4.70 (s, 2H), 4.57 (s, 2H), 3.27-3.30 (m, 1H), 3.12-3.16 (m, 1H), 1.82-1.67 (m, 2H), 1.56-1.40 (m, 2H), 0.96 (t, J = 7.2 Hz, 3H). | Exact mass: 422.12, found: 423.3 | R-isomer: 99.0% ee; [α] = +40.238° |
| 63 | 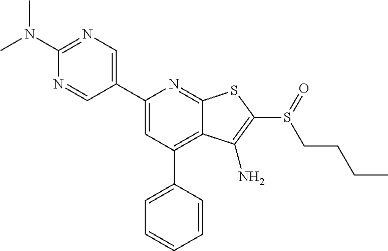 [SMILES: CCCCS(=O)c1sc2nc(-c3cnc(N(C)C)nc3)cc(-c3ccccc3)c2c1N] | δ 9.04 (s, 2H), 7.49-7.55 (m, 5H), 7.39 (s, 1H), 4.57 (s, 2H), 3.34-3.29 (m, 1H), 3.28 (s, 6H), 3.08-3.11 (m, 1H), 1.71-1.77 (m, 2H), 1.48-1.53 (m, 2H), 0.96 (t, J = 7.2 Hz, 3H). | Exact mass: 451.15, found: 452.3 | |
| 64 | 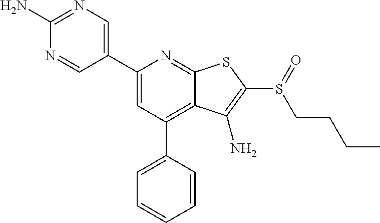 [SMILES: CCCCS(=O)c1sc2nc(-c3cnc(N)nc3)cc(-c3ccccc3)c2c1N] | δ 9.04 (s, 2H), 7.63-7.45 (m, 5H), 7.43 (s, 1H), 5.33 (s, 2H), 4.60 (s, 2H), 3.36-3.07 (m, 2H), 1.80-1.67 (m, 2H), 1.57-1.42 (m, 2H), 0.96 (t, J = 7.2 Hz, 3H). | Exact mass: 423.12, found: 424.1 | R-isomer: 99.0% ee; [α] = +42.895° |
| 65 | 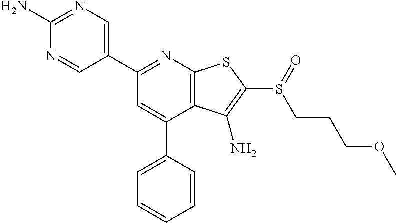 [SMILES: COCCCS(=O)c1sc2nc(-c3cnc(N)nc3)cc(-c3ccccc3)c2c1N] | δ 9.03 (s, 2H), 7.56-7.55 (m, 3H), 7.49-7.42 (m, 2H), 7.42 (s, 1H), 5.32 (s, 2H), 4.60 (s, 2H), 3.54-3.48 (m, 2H), 3.41- 3.32 (m, 4H), 3.28-3.19 (m, 1H), 2.06-2.01(m, 2H). | Exact mass: 439.11, found: 440.1 | R-isomer: 95.62% ee; [α] = +48.875° |
| 66 | 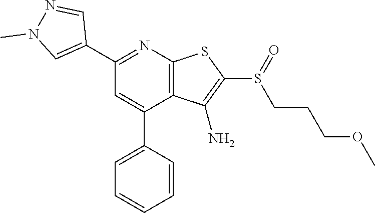 | δ 8.05 (s, 1H), 8.00 (s, 1H), 7.58-7.43 (m, 5H), 7.27 (s, 1H), 4.55 (s, 2H), 3.98 (s, 3H), 3.56-3.46 (m, 2H), 3.41- 3.31 (m, 4H), 3.26-3.15 (m, 1H), 2.04-1.98 (m, 2H). | Exact mass: 426.12, found: 427.2 | |
| 67 | 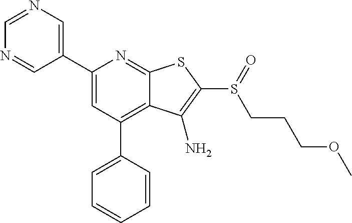 [SMILES: COCCCS(=O)c1sc2nc(-c3cncnc3)cc(-c3ccccc3)c2c1N] | 9.44 (s, 2H), 9.30 (s, 1H), 7.57-7.59 (m, 4H), 7.51 (s, 2H), 4.67 (s, 2H), 3.52 (t, J = 6.4 Hz, 2H), 3.36-3.41 (m, 1H), 3.35 (s, 3H), 3.21-3.28 (m, 1H), 1.99-2.14 (m, 2H) | Exact mass: 424.10, found: 425.0 | R-isomer: 99.0% ee; [α] = +103.630° |
| 68 | 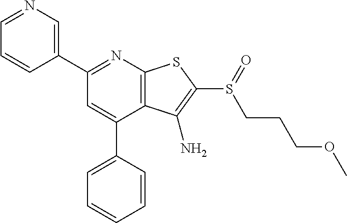 [SMILES: COCCCS(=O)c1sc2nc(-c3cccnc3)cc(-c3ccccc3)c2c1N] | 9.29 (d, J = 1.6 Hz, 1H), 8.69 (dd, J₁ = 1.6 Hz, J₂ = 4.8 Hz, 1H), 8.44-8.47 (m, 1H), 7.60 (s, 1H), 7.56 (d, J = 2.4 Hz, 3H), 7.51 (s, 2H), 7.42-7.46 (m, 1H), 4.64 (s, 2H), 3.52 (t, J = 5.6 Hz, 2H), 3.34-3.41 (m, 1H), 3.34 (s, 3H), 3.20-3.27 (m, 1H), 2.00-2.07 (m, 2H). | Exact mass: 423.11, found: 424.0 | R-isomer: 98.0% ee; [α] = +96.504° |
| 69 | 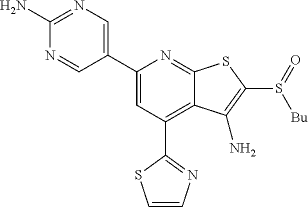 | □□□□□□(s, 2H), 8.25 (d, J = 2.8 Hz, 2H), 8.18 (s, 1H), 7.27 (s, 2H), 6.83 (s, 2H), 3.17-3.21 (m, 1H), 3.08-3.10 (m, 1H), 1.40-1.60 (m. 4H), 0.95 (t, J = 7.2 Hz, 3H) | Exact mass: 430.07, found: 431.1 | |
| 90 | 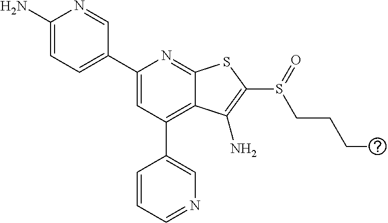 | δ 8.76-8.82 (m, 3H), 8.29 (dd, J₁= 2.4 Hz, J₂ = 8.8 Hz, 1H), 7.86 ( d, J = 7.6 Hz, 1H), 7.50-7.52 (m, 1H), 7.45 (s, 1H), 6.65 (d, J = 8.4 Hz, 1H), 4.92 (s, 2H), 4.49 ( s, 2H), 3.52 (t, J = 6.0 Hz, 2H), 3.34- 3.36 (m, 4H), 3.22- 3.27(m,1H), 2.01-2.05 (m, 2H). | Exact mass: 440.12 (M + H)⁺ found: m/z = 440.0 | |
| 91 | 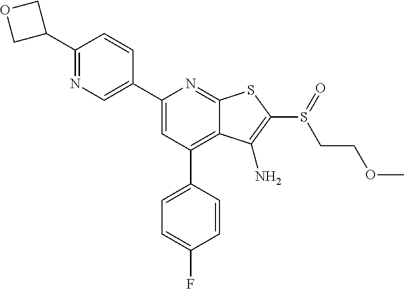 [SMILES: COCCS(=O)c1sc2nc(-c3ccc(C4COC4)nc3)cc(-c3ccc(F)cc3)c2c1N] | 9.28 (d, J = 2.0 Hz, 1H), 8.46 (dd, J₁ = 2.0 Hz, J₂ = 8.0 Hz, 1H), 7.57 (s, 1H), 7.50- 7.52 (m, 2H), 7.44 (d, J = 8.0 Hz, 1H), 7.30 (s, 1H), 7.25 (s, 1H), 5.10-5.12 (m, 2H), 4.99 (t, J = 6.4 Hz, 2H), 4.59 (s, 2H), 4.44-4.45 (m, 1H), 3.85-3.88 (m, 1H), 3.70-3.72 (m, 1H), 3.60-3.61 (m, 1H) 3.41 (s, 3H), 3.29-3.40 (m, 1H). | Exact mass: 484.1 (M + H)⁺ Found = 484.0 | |

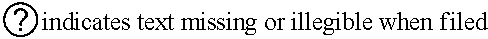

### Example 70-1: 2-bromo-1-oxazol-5-yl-ethanone

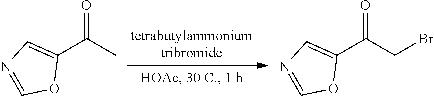

To a solution of 1-oxazol-5-ylethanone (1.4 g, 12.6 mmol, 1.0 eq) in acetic acid (10 mL) was added tetrabutylammonium tribromide (6.1 g, 12.6 mmol, 1.0 eq). The mixture was stirred at 30° C. for 1 hour. The reaction mixture was partitioned between ethyl acetate (50 mL) and sodium bicarbonate solution (50 mL). The organic phase was separated, washed with brine 60 mL (20 mL*3), dried over anhydrous sodium sulfate, filtered and concentrated under reduced pressure to give a residue. The residue was purified by column chromatography (SiO₂, Petroleum ether/Ethyl acetate=10/1 to 3:1) to give the target compound (1.46 g, 61% yield) as a brown solid. ¹H NMR (400 MHz, CDCl₃) δ 8.06 (s, 1H), 7.95-7.92 (m, 1H), 4.52-4.25 (m, 2H)

### Example 70-2: 1-oxazol-5-yl-2-(triphenylphosphanylidene)ethanone

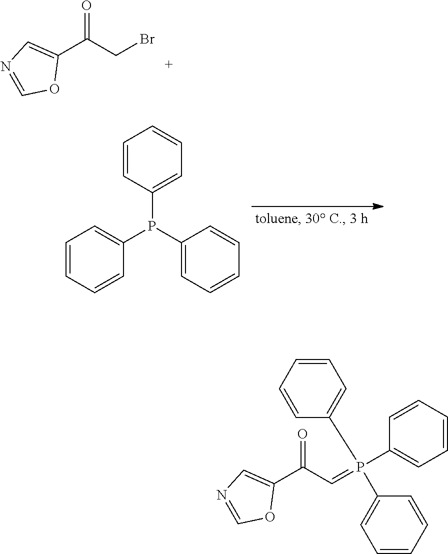 [SMILES: O=C(CBr)c1cnco1 ; c1ccc(P(c2ccccc2)c2ccccc2)cc1 ; O=C(C=P(c1ccccc1)(c1ccccc1)c1ccccc1)c1cnco1]

To a solution of Example 70-1 (1.36 g, 7.2 mmol, 1.0 eq) in toluene (20 mL) was added triphenylphosphine (2.07 g, 7.9 mmol, 1.1 eq). The mixture was stirred at 30° C. for 3 hours. The yellow precipitate was removed by filtration and was washed with toluene (10 mL) and petroleum ether (10 mL). Water (30 mL) was added to the precipitate and the mixture treated with 1N lithium hydroxide to pH=10. The mixture was stirred for 30 minutes at room temperature. The precipitate was removed by filtration and washed with water (20 mL) to give the target compound (1.56 g, 59% yield) as a light brown solid which was used in the next step without further purification or characterization.

### Example 70-3: (E)-1-oxazol-5-yl-3-phenyl-prop-2-en-1-one

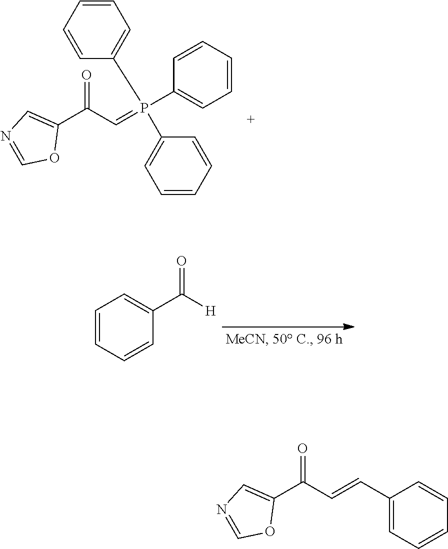 [SMILES: O=C(C=P(c1ccccc1)(c1ccccc1)c1ccccc1)c1cnco1 ; O=Cc1ccccc1 ; O=C(/C=C/c1ccccc1)c1cnco1]

A mixture of benzaldehyde (490 mg, 4.6 mmol, 467 μL, 1.1 eq) and Example 70-2 (1.56 g, 4.2 mmol, 1.0 eq) in acetonitrile (20 mL) was degassed and purged with nitrogen 3 times. The mixture was stirred at 50° C. for 96 hours under a nitrogen atmosphere. The mixture was concentrated. The residue was purified by column chromatography (SiO₂, Petroleum ether/Ethyl acetate=10/1 to 5:1) to give the target compound (600 mg, 72% yield) as a light yellow solid.

¹H NMR (400 MHz, CDCl₃) δ 8.09 (s, 1H), 7.96-7.93 (m, 2H), 7.69-7.66 (m, 2H), 7.47-7.45 (m, 3H), 7.33 (d, J=15.6 Hz, 1H).

### Example 70-4: 6-oxazol-5-yl-4-phenyl-2-sulfanyl-pyridine-3-carbonitrile

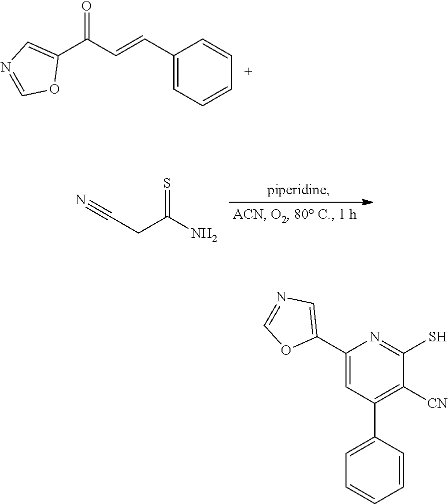 [SMILES: O=C(/C=C/c1ccccc1)c1cnco1 ; N#CCC(N)=S ; N#Cc1c(-c2ccccc2)cc(-c2cnco2)nc1S]

Example 70-4 was prepared using General Procedure A, Step 2 starting from (E)-1-oxazol-5-yl-3-phenyl-prop-2-en-1-one (550 mg) and 2-cyanothioacetamide (829 mg) to give the target compound (770 mg, crude) as a red oil which was used in the next step without further purification or characterization.

### Example 70-5: 2-(3-methoxypropylsulfanylmethylsulfanyl)-6-oxazol-5-yl-4-phenyl-pyridine-3-carbonitrile

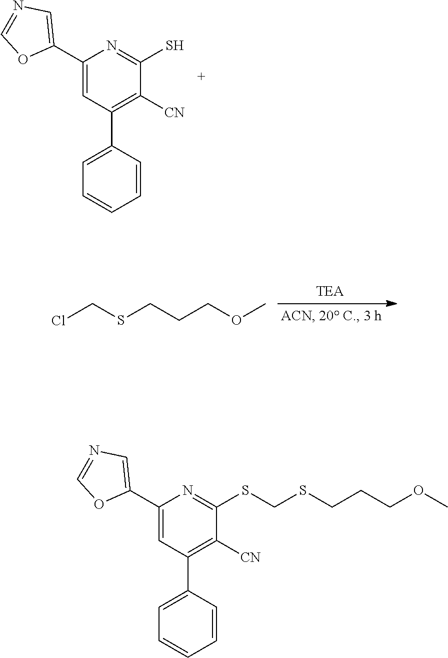

Example 70-5 was prepared using procedure using General Procedure A, Step 4 starting from 6-oxazol-5-yl-4-phenyl-2-sulfanyl-pyridine-3-carbonitrile (385 mg) and 1-(chloromethylsulfanyl)-3-methoxy-propane (426 mg) to give the target compound (240 mg, 44% yield) as a light yellow solid.

¹H NMR (400 MHz, CDCl₃) δ 8.04 (s, 1H), 7.89 (s, 1H), 7.67-7.61 (m, 2H), 7.58-7.54 (m, 3H), 7.49 (s, 1H), 4.51 (s, 2H), 3.49 (t, J=6.0 Hz, 2H), 3.35 (s, 3H), 2.88-2.82 (m, 2H), 1.99-1.90 (m, 2H).

### Example 70-6: 2-(3-methoxypropylsulfinylmethylsulfanyl)-6-oxazol-5-yl-4-phenyl-pyridine-3-carbonitrile

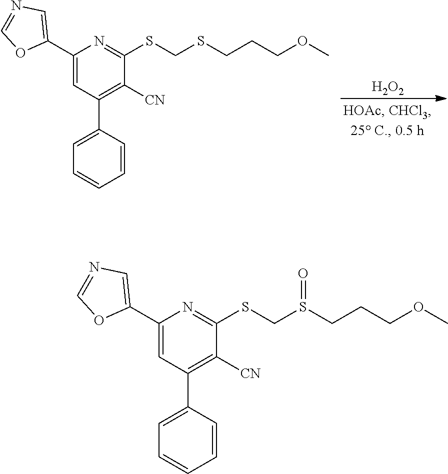

Example 70-6 was prepared using General Procedure A, Step 5 starting from 2-(3-methoxypropylsulfanylmethylsulfanyl)-6-oxazol-5-yl-4-phenyl-pyridine-3-carbonitrile (220 mg) to give the target compound (220 mg, 96% yield) as a yellow solid.

### Example 70: 2-(3-methoxypropylsulfinyl)-6-oxazol-5-yl-4-phenyl-thieno[2,3-b]pyridin-3-amine

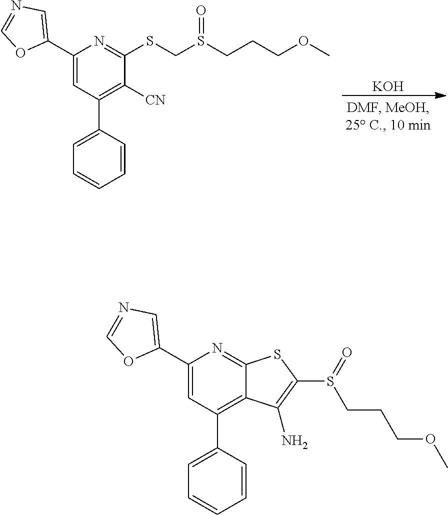

Example 70 was prepared using General Procedure A, Step 6 starting from 2-(3-methoxypropylsulfinylmethylsulfanyl)-6-oxazol-5-yl-4-phenyl-pyridine-3-carbonitrile (210 mg) to give the target compound (110 mg, 52% yield) as a yellow solid.

LCMS: (ES⁺) m/z (M+H)⁺=414.1 ¹H NMR (400 MHz, CDCl₃) δ 8.01 (s, 1H), 7.83 (s, 1H), 7.60-7.56 (m, 3H), 7.51 (s, 1H), 7.50-7.45 (m, 2H), 4.63 (s, 2H), 3.53-3.50 (m, 2H), 3.42-3.35 (m, 1H), 3.34 (s, 3H), 3.29-3.18 (m, 1H), 2.07-2.00 (m, 2H).

### Example 71-1: (E)-1-(pyridin-3-yl)-3-(pyridin-4-yl)prop-2-en-1-one

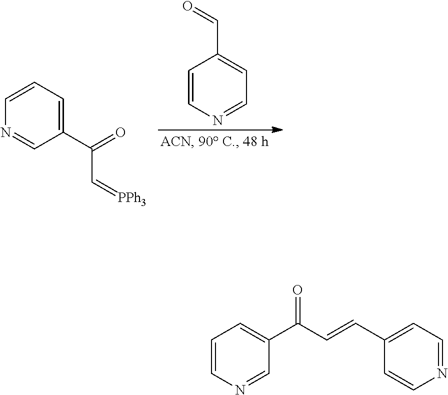

Example 71-1 was prepared by the procedure used for Example 70-3 starting from 1-(3-pyridyl)-2-(triphenyl-phosphanylidene)ethanone (650 mg, 1.70 mmol, 1.0 eq) and pyridine-4-carbaldehyde (182.54 mg, 1.70 mmol, 160.12 uL, 1 eq) to give the target compound (160 mg, 45% yield) as yellow solid. ¹H NMR (400 MHz, CDCl₃) δ 9.25 (d, J=1.6 Hz, 1H), 8.84-8.86 (m, 1H), 8.72-8.74 (m, 2H), 8.30-8.33 (m, 1H), 7.76 (d, J=15.6 Hz, 1H), 7.63 (d, J=15.6 Hz, 1H), 7.49-7.52 (m, 3H).

### Example 71-2:6′-thioxo-1′,6′-dihydro-[3,2′:4′,4″-terpyridine]-5′-carbonitrile

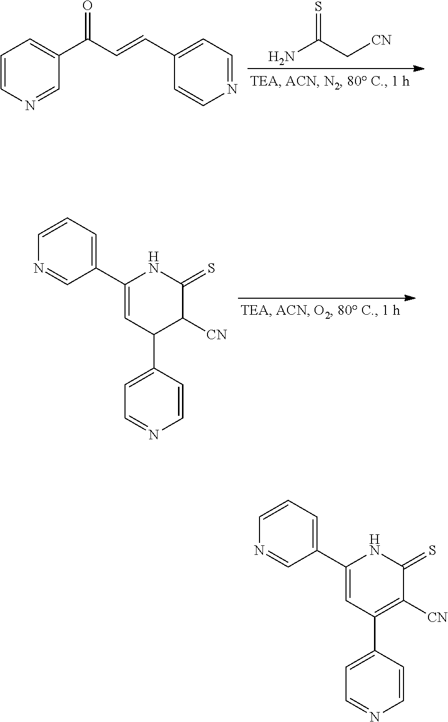 [SMILES: O=C(/C=C/c1ccncc1)c1cccnc1 ; N#CCC(N)=S ; N#CC1C(=S)NC(c2cccnc2)=CC1c1ccncc1 ; N#Cc1c(-c2ccncc2)cc(-c2cccnc2)[nH]c1=S]

To a solution of (E)-1-(pyridin-3-yl)-3-(pyridin-4-yl)prop-2-en-1-one (134 mg, 637 umol, 1.0 eq) in acetonitrile (5 mL) was added triethylamine (1.27 mmol, 176 μL, 2.0 eq) and 2-cyanothioacetamide (128 mg, 1.27 mmol, 2.0 eq). The mixture was stirred at 80° C. for 1 hour under a nitrogen atmosphere. Then the mixture was stirred at 80° C. for 3 hr under oxygen (15 psi). After cooling, the mixture was concentrated to give the target compound as black oil which was used in the next step without further purification or characterization.

### Example 71-3:6′-(((butylthio)methyl)thio)-[3,2′:4′,4″-terpyridine]-5′-carbonitrile

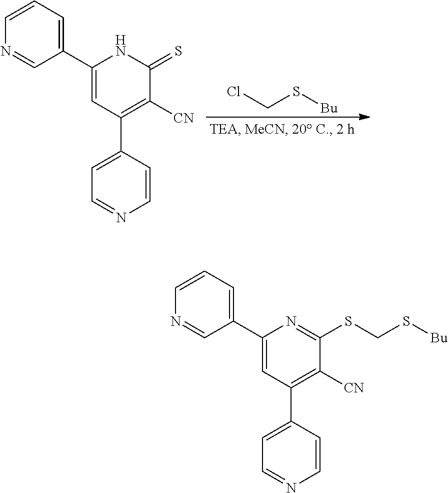

To a solution of 6′-thioxo-1′,6′-dihydro-[3,2′:4′,4″-terpyridine]-5′-carbonitrile (185 mg, 637 umol, 1.0 eq) in acetonitrile (3 mL) was added triethylamine (1.27 mmol, 176 μL, 2.0 eq) and 1-(chloromethylsulfanyl)butane (177 mg, 1.27 mmol, 2.0 eq). The mixture was stirred at 25° C. for 15 minutes. The mixture was concentrated and the crude product was purified by prep-HPLC (column: Phenomenex Synergi C18 150*25*10 um; mobile phase: [water (0.1% TFA)-ACN]; B %: 24%-54%, 13 min) to give to give the target compound (250 mg, 100% yield) as a gray solid.

¹H NMR (400 MHz, CDCl₃) δ 9.39 (s, 1H), 8.90 (d, J=1.6 Hz, 2H), 8.82 (d, J=3.6 Hz, 1H), 8.63 (d, J=8.4 Hz, 1H), 7.67-7.69 (m, 1H), 7.65 (d, J=6.00 Hz, 2H), 7.61 (s, 1H), 4.55 (s, 2H), 2.78 (t, J=6.0 Hz, 2H), 1.63-1.71 (m, 2H), 1.41-1.50 (m, 2H), 0.94 (t, J=7.2 Hz, 3H).

### Example 71-4: 6′-(((butylsulfinyl)methyl)thio)-[3,2′:4′,4″-terpyridine]-5′-carbonitrile

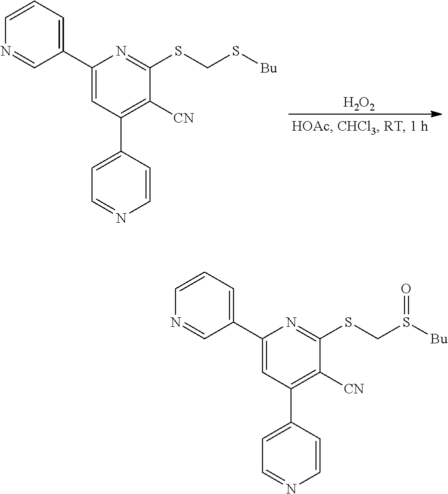

Example 71-4 was prepared using General Procedure A, Step 5 starting from 6′-(((butylthio)methyl)thio)-[3,2′:4′,4″-terpyridine]-5′-carbonitrile (250 mg, 636.88 umol, 1.0 eq) and hydrogen peroxide (144 mg, 1.27 mmol, 122 μL, 30% purity, 2.0 eq) to give the target compound (200 mg, 77% yield) as white solid which was used in the next step without further purification or characterization.

### Example 41:2-(butylsulfinyl)-6-(pyridin-3-yl)-4-(pyridin-4-yl)thieno[2,3-b]pyridin-3-amine

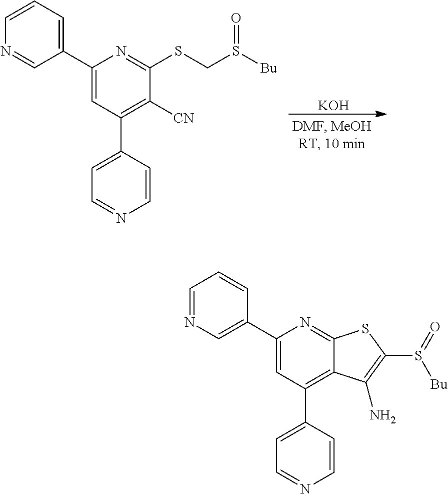

Example 71 was prepared using General Procedure A, Step 6 starting from 6′-(((butylsulfinyl)methyl)thio)-[3,2′:4′,4″-terpyridine]-5′-carbonitrile (180 mg, 440 umol, 1.0 eq) to give the target compound (120 mg, 66% yield,) as yellow solid.

LCMS: (ES⁺) m/z (M+H)⁺=409.2 ¹H NMR (400 MHz, CDCl₃) δ 9.29 (d, J=2.0 Hz, 1H), 8.85 (d, J=5.6 Hz, 2H), 8.70-8.72 (m, 1H), 8.44-8.47 (m, 1H), 7.57 (s, 1H), 7.48 (d, J=6.4 Hz, 2H), 7.45 (d, J=8.0 Hz, 1H), 4.59 (s, 2H), 3.33-3.35 (m 1H), 3.1-3.18 (m, 1H), 1.73-1.81 (m, 2H), 1.45-1.55 (m, 2H), 0.97 (t, J=7.2 Hz, 3H).

### Example 72-1: 2-chloro-5-(1-ethoxyvinyl)pyrimidine

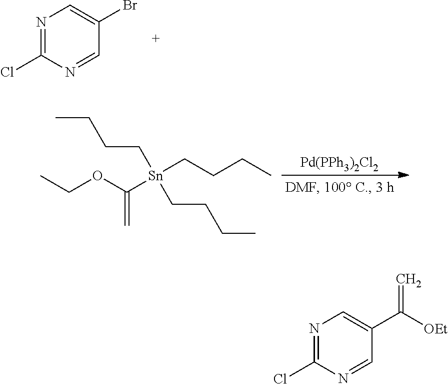 [SMILES: Clc1ncc(Br)cn1 ; C=[C](OCC)[Sn]([CH2]CCC)([CH2]CCC)[CH2]CCC ; C=C(OCC)c1cnc(Cl)nc1 ; "Pd(PPh3)2Cl2"]

To a solution of 5-bromo-2-chloro-pyrimidine (20 g, 103 mmol, 1.0 eq) in N,N-dimethylformamide (200 mL) was added tributyl(1-ethoxyvinyl)stannane (37.3 g, 103.40 mmol, 34.9 mL, 1.0 eq) and dichlorobis(triphenylphosphine)palladium(II) (3.63 g, 5.17 mmol, 0.05 eq). The mixture was stirred at 100° C. for 3 hours under a nitrogen atmosphere. The mixture was then diluted with ethyl acetate (200 mL) and treated with aqueous potassium fluoride solution (88 g of potassium fluoride in 60 mL of water). The two phase mixture was stirred vigorously for 1 h at room temperature before being filtered through diatomaceous earth (Celite®). The filtrate was washed with saturated sodium bicarbonate solution and brine prior to drying (anhydrous sodium sulfate). The original aqueous phase was extracted with ethyl acetate (2×50 mL) and the organic phase was treated as above. The combined organic solutions were concentrated to give the target compound (15 g, 79% yield) as a yellow solid.

¹H NMR (400 MHz, CDCl₃) δ 8.81 (s, 2H), 4.73 (d, J=3.6 Hz, 1H), 4.40 (d, J=3.2 Hz, 1H), 3.96 (m, 2H), 1.43 (t, J=7.2 Hz, 3H).

### Example 72-2: 1-(2-chloropyrimidin-5-yl)ethanone

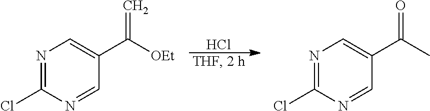

To a solution of Example 72-1 (15 g, 81.25 mmol, 1.0 eq) in tetrahydrofuran (200 mL) was added hydrochloric acid (1 M, 122 mL, 1.5 eq). The mixture was stirred at 20° C. for 2 hours. The mixture was concentrated and the residue partitioned between saturated aqueous sodium bicarbonate (150 mL) and ethyl acetate (100 mL). The organic layer was washed with brine (50 mL*3), dried over anhydrous sodium sulfate, filtered and concentrated. The residue was purified by column chromatography (silica gel, Petroleum ether/Ethyl acetate=10/1 to 5:1) to give the target compound (10 g, 79% yield) as a light yellow solid.

¹H NMR (400 MHz, CDCl₃) δ 9.10 (s, 2H), 2.64 (s, 3H)

### Example 72-3: methyl 5-acetylpyrimidine-2-carboxylate

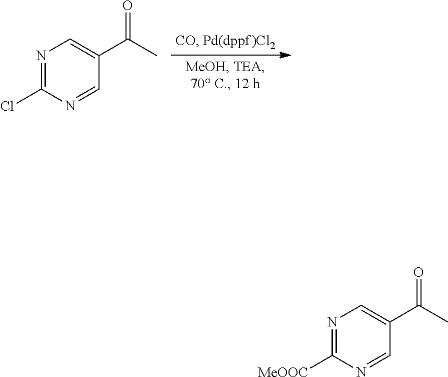

Carbon monoxide (50 psi) was bubbled into a mixture of Example 72-2 (4.5 g, 28.74 mmol, 1.0 eq), 1,1′-bis(diphenylphosphino)ferrocene-palladium(II)dichloride (1.05 g, 1.4 mmol, 0.05 eq) and triethylamine (5.8 g, 58 mmol, 8.0 mL, 2.0 eq) in methanol (100 mL) at 70° C. for 12 hours. The mixture was concentrated. The residue was purified by column chromatography (silica gel, Petroleum ether/Ethyl acetate=10/1 to 2:1) to give the target compound (1.5 g, 29% yield) as a light yellow solid. ¹H NMR (400 MHz, CDCl₃) δ 9.37 (s, 2H), 4.10 (s, 3H), 2.71 (s, 3H).

### Example 72-4: ethyl 5-[(E)-3-(2,3-dimethylimidazol-4-yl)prop-2-enoyl]pyrimidine-2-carboxylate

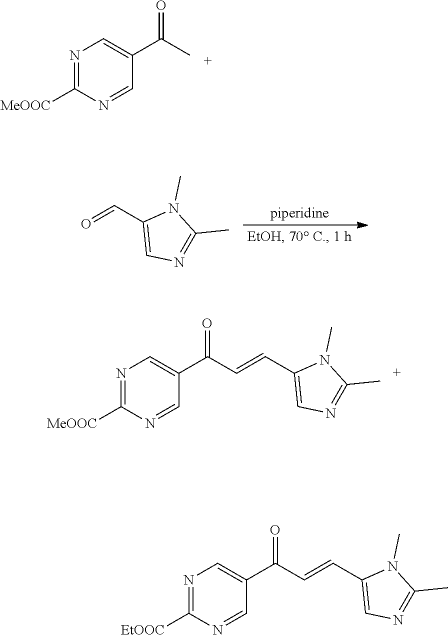

Example 72-4 was prepared using General Procedure A, Step 1 starting from methyl 5-acetylpyrimidine-2-carboxylate (1.5 g) and 2,3-dimethylimidazole-4-carbaldehyde (1.0 g) to give the target compound (830 mg, 33% yield) as a yellow solid. ¹H NMR (400 MHz, CDCl₃) δ 9.41 (s, 2H), 7.78 (d, J=15.⁶ Hz, 1H), 7.69 (s, 1_{H}), 7.23 (s, 1H), 4.59 (q, J=7.2 Hz, 2H), 3.68 (s, 3H), 2.49 (s, 3H), 1.50 (t, J=7.2 Hz, 3H).

### Example 72-6: ethyl 5-[5-cyano-4-(2,3-dimethylimidazol-4-yl)-⁶-sulfanyl-2-pyridyl]ₚyrimidine-2-carboxylate

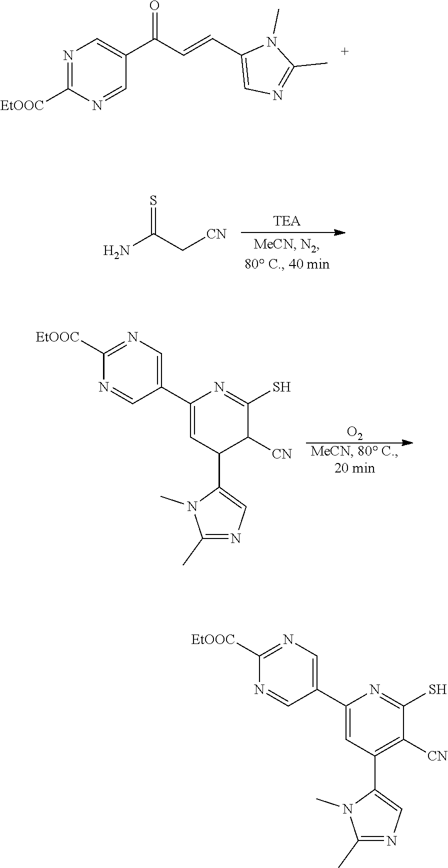 [SMILES: CCOC(=O)c1ncc(C(=O)/C=C/c2cnc(C)n2C)cn1 ; N#CCC(N)=S ; CCOC(=O)c1ncc(C2=CC(c3cnc(C)n3C)C(C#N)C(S)=N2)cn1 ; "O2" ; "MeCN, 80° C.,20 min" ; CCOC(=O)c1ncc(-c2cc(-c3cnc(C)n3C)c(C#N)c(S)n2)cn1]

Example 72-6 was prepared using the procedure for Example 71-2 starting from 5-[(E)-3-(2,3-dimethylimidazol-4-yl)prop-2-enoyl]pyrimidine-2-carboxylate (800 mg) and 2-cyanothioacetamide (800 mg) to give the target compound (1 g, crude) as a deep red solution (in acetonitrile) which was used in the next step with no further purification. LCMS: M+1 (381.1); MW (380.4).

### Example 42-7: ethyl 5-[6-(butylsulfanylmethylsulfanyl)-5-cyano-4-(2,3-dimethylimidazol-4-yl)-2-pyridyl]pyrimidine-2-carboxylate

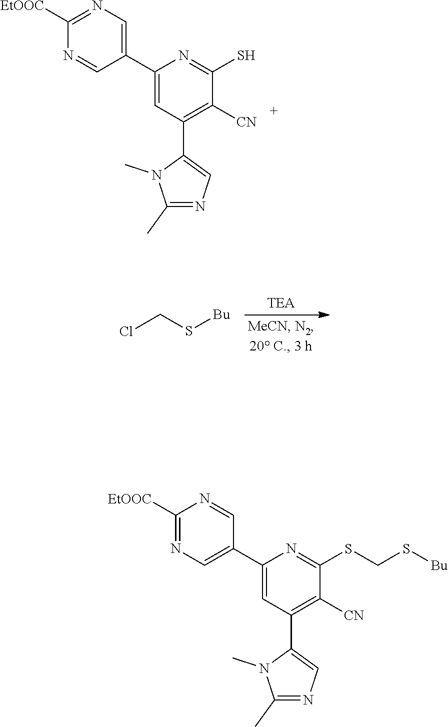

Example 72-7 was prepared using the procedure for Example 71-3 starting from 5-[5-cyano-4-(2,3-dimethylimidazol-4-yl)-6-sulfanyl-2-pyridyl]pyrimidine-2-carboxylate (500 mg) and 1-(chloromethylsulfanyl)butane (273 mg) to give the target compound (180 mg, 28% yield) as a light brown solid. 1H NMR (400 MHz, CDCl₃) δ 9.58 (s, 2H), 7.53 (s, 1H), 7.40 (s, 1H), 4.60 (m, 2H), 4.48 (s, 2H), 3.64 (s, 3H), 2.76 (t, J=7.4 Hz, 2H), 2.55 (s, 3H), 1.65-1.66 (m, 2H), 1.52 (m, 3H), 1.41-1.49 (m, 2H), 0.94 (t, J=7.4 Hz, 3H).

### Example 72-8:5-[6-(butylsulfanylmethylsulfanyl)-5-cyano-⁴-(2,3-dimethylimidazol-4-yl)-2-pyridyl]pyrimidine-2-carboxylic acid

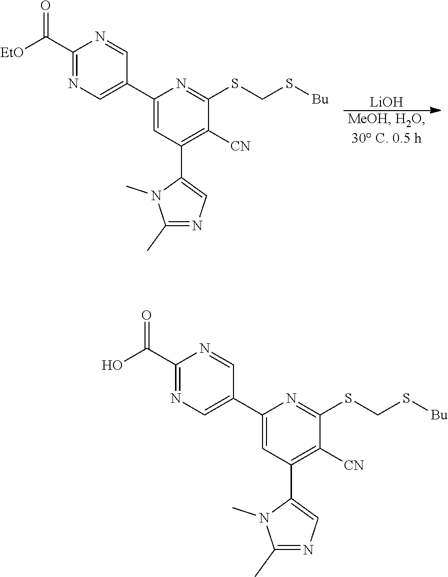

To a solution of Example 72-7 (180 mg, 373 umol, 1.0 eq) in methanol (3 mL) and water (1 mL) was added lithium hydroxide monohydrate (31 mg, 746 umol, 2.0 eq). The mixture was stirred at 30° C. for 0.5 hour. The mixture was cooled to 0° C. and then acidified to pH 7 with 1N hydrochloric acid. The solid was filtered to give the target compound (160 mg, 94% yield) as a yellow solid. ¹H NMR (400 MHz, DMSO-d₆) δ 9.72 (s, 2H), 8.17 (s, 1H), 7.54 (s, 1H), 4.69 (s, 2H), 3.68 (s, 3H), 2.73-2.69 (t, J=7.2 Hz, 2H), 1.58-1.62 (m, 2H), 1.31-1.40 (m, 2H), 0.86-0.83 (t, J=7.2 Hz, 3H).

### Example 72-9:6-(2-aminopyrimidin-5-yl)-2-(butylsulfanylmethylsulfanyl)-4-(2,3-dimethylimidazol-4-yl)pyridine-3-carbonitrile

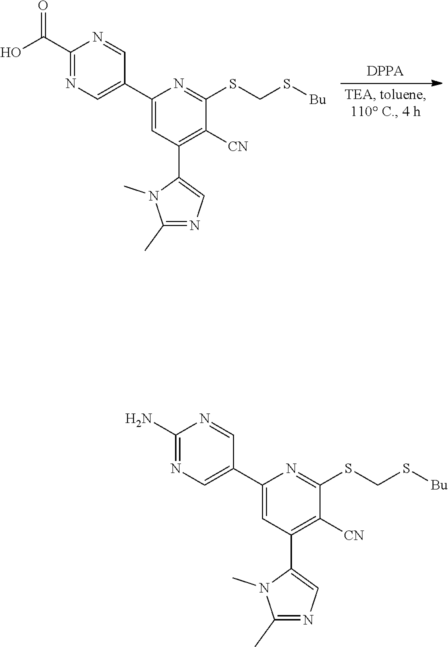

To a solution of Example 72-8 (140 mg, 308 umol, 1.0 eq) in toluene (10 mL) was added triethylamine (62 mg, 616 umol, 86 μL, 2.0 eq), followed by diphenylphosphoryl azide (170 mg, 616 umol, 133 μL, 2.0 eq). The mixture was stirred at 110° C. for 4 hours under a nitrogen atmosphere. The mixture was concentrated. The residue was purified by prep-HPLC (column: Waters Xbridge 150*25 5 u; mobile phase: [water (0.05% ammonia hydroxide v/v)-ACN]; B %: 32%-59%, 10 min) to give the target compound (50 mg, 38% yield) as a light yellow solid. ¹H NMR (400 MHz, DMSO-d₆) δ 9.11 (s, 2H), 7.77 (s, 1H), 7.30-7.34 (m, 2H), 7.28 (s, 1H), 4.63 (s, 2H), 3.62 (s, 3H), 2.68-2.74 (m, 2H), 2.42 (s, 3H), 1.54-1.62 (m, 2H), 1.32-1.38 (m, 2H), 0.87-0.84 (t, J=7.2 Hz, 3H).

### Example 72-10:6-(2-aminopyrimidin-5-yl)-2-(butylsulfinylmethylsulfanyl)-4-(2,3-dimethylimidazol-4-yl)pyridine-3-carbonitrile

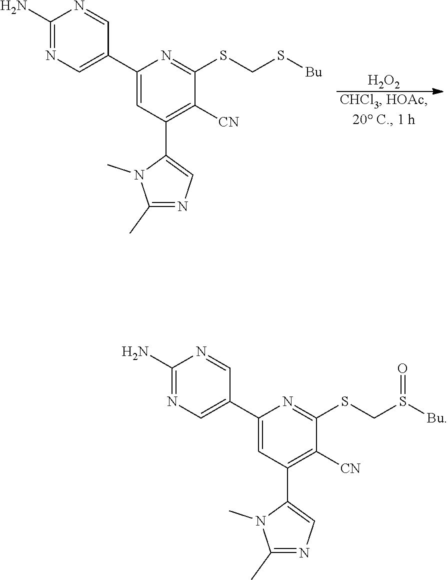

Example 72-10 was prepared using General Procedure A, Step 5 starting from 6-(2-aminopyrimidin-5-yl)-2-(butylsulfanylmethylsulfanyl)-4-(2,3-dimethylimidazol-4-yl)pyridine-3-carbonitrile (50 mg) and hydrogen peroxide (20 mg, 1.5 eq) to give the target compound (50 mg, 96% yield) as a yellow solid. ¹H NMR (400 MHz, CDCl₃) δ 9.00 (s, 2H), 7.33 (s, 2H), 5.44 (s, 2H), 4.68 (d, J=13.2 Hz, 1H), 4.49 (d, J=13.2 Hz, 1H), 3.62 (s, 3H), 2.80-2.98 (m, 2H), 2.51 (s, 3H), 1.78-1.85 (m, 2H), 1.52-1.60 (m, 2H), 0.99-0.96 (t, J=7.4 Hz, 3H).

### Example 72:6-(2-aminopyrimidin-5-yl)-2-butylsulfinyl-4-(2,3-dimethylimidazol-₄-yl)thieno[2,3-b]pyridin-3-amine

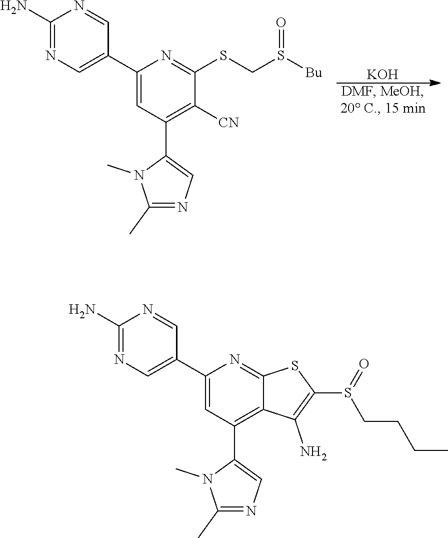

Example 72 was prepared using General Procedure A, Step 6 starting from 6-(2-aminopyrimidin-5-yl)-2-(butylsulfinylmethylsulfanyl)-4-(2,3-dimethylimidazol-4-yl)pyridine-3-carbonitrile (50 mg) to give the target compound (21 mg, 41% yield) as a yellow solid.

LCMS: (ES⁺) m/z (M+H)⁺=442.2. ¹H NMR (400 MHz, CDCl₃) δ 9.03 (s, 2H), 7.44 (s, 1H), 7.13 (s, 1H), 5.35 (s, 2H), 4.68 (s, 2H), 3.40 (s, 3H), 3.28-3.30 (m, 1H), 3.12-3.15 (m, 1H), 2.54 (s, 3H), 1.72-1.83 (m, 2H), 1.50-1.53 (m, 2H), 0.98-0.94 (t, J=7.4 Hz, 3H).

### Example 73-1: 5-methylthiazole-4-carbaldehyde

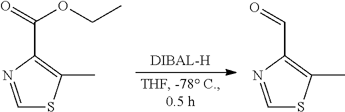

To a solution of ethyl 5-methylthiazole-4-carboxylate (900 mg, 5.26 mmol, 1 eq) in tetrahydrofuran (25 mL) was added diisobutylaluminum hydride (1 M, 6.31 mL, 1.2 eq). The mixture was stirred at −78° C. for 30 minutes. The reaction mixture was quenched with water (2 mL) at −78° C. The reaction was warmed to room temperature. The reaction was poured into 40 mL of saturated potassium sodium tartrate in water and diluted with ethyl acetate. The layers were stirred for 1 hour and the aqueous layer was extracted with ethyl acetate 60 mL (20*3 mL). The combined organics were washed with brine, dried over anhydrous sodium sulfate, filtered and concentrated in vacuo. The residue was purified by column chromatography (SiO₂, petroleum ether: ethyl acetate=8:1 to 7:1) to give the target compound (230 mg, 34% yield) as a yellow liquid. ¹H NMR (400 MHz, CDCl₃) δ 10.11 (s, 1H), 8.52 (s, 1H), 2.72 (s, 3H).

### Example 73-2: (E)-3-(5-methylthiazol-4-yl)-1-(pyridin-3-yl)prop-2-en-1-one

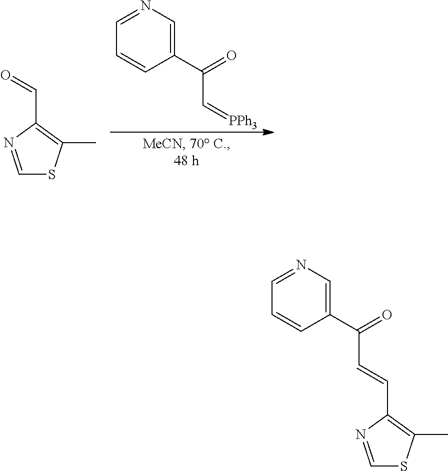

Example 73-2 was prepared by the procedure used for Example 70-3 starting from 1-(3-pyridyl)-2-(triphenyl-phosphanylidene)ethanone (600 mg, 1.57 mmol, 1 eq) and 5-methylthiazole-4-carbaldehyde (200 mg, 1.57 mmol, 1 eq) to give the target compound (250 mg, 69% yield) as yellow solid. ¹H NMR (400 MHz, CDCl₃) δ 9.31 (d, J=1.6 Hz, 1H), 8.80-8.82 (m, 1H), 8.69 (s, 1H), 8.35-8.38 (m, 1H), 7.91 (d, J=2.8 Hz, 2H), 7.53-7.70 (m, 1H), 7.45-7.48 (m, 1H), 2.65 (s, 3H).

### Example 73-3: 6-mercapto-4-(5-methylthiazol-4-yl)-4,5-dihydro-[2,3′-bipyridine]-5-carbonitrile

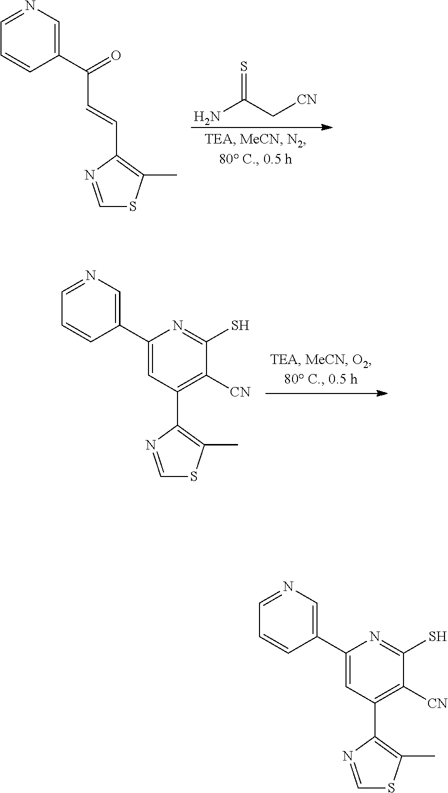 [SMILES: Cc1scnc1/C=C/C(=O)c1cccnc1 ; N#CCC(N)=S ; Cc1scnc1-c1cc(-c2cccnc2)nc(S)c1C#N ; Cc1scnc1-c1cc(-c2cccnc2)nc(S)c1C#N]

Example 73-3 was prepared using the procedure for Example 71-2 starting from (E)-3-(5-methylthiazol-4-yl)-1-(pyridin-3-yl)prop-2-en-1-one (240 mg, 1.04 mmol, 1 eq) and 2-cyanothioacetamide (157 mg, 1.56 mmol, 1.5 eq) to give the crude target compound as black oil which was used in the next step without further purification or characterization.

### Example 73-4: 6-(((butylthio)methyl)thio)-4-(5-methylthiazol-4-yl)-[2,3′-bipyridine]-5-carbonitrile

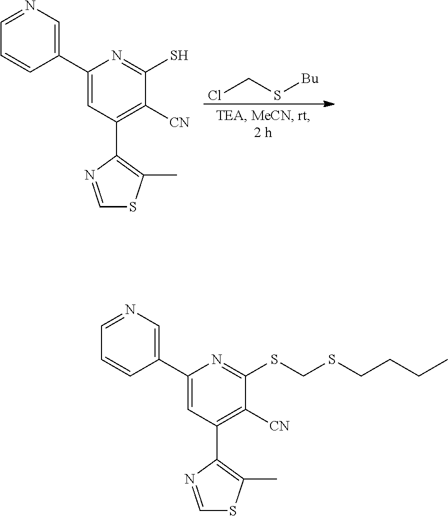

Example 73-4 was prepared using the procedure for compound Example 71-3 starting from 6-mercapto-4-(5-methylthiazol-4-yl)-4,5-dihydro-[2,3′-bipyridine]-5-carbonitrile_(325 mg, 1.05 mmol, 1 eq) and 1-(chloromethylsulfanyl)butane (218 mg, 1.57 mmol, 1.5 eq) to give the target compound (280 mg, 65% yield) as a gray solid. ¹H NMR (400 MHz, CDCl₃) δ 9.46 (s, 1H), 8.83-8.86 (m, 2H), 8.81 (s, 1H), 7.82-7.85 (m, 1H), 7.77 (s, 1H), 4.52 (s, 2H), 2.78 (d, J=7.2 Hz, 2H), 2.64 (s, 3H), 1.62-1.70 (m, 2H), 1.70-1.48 (m, 2H), 0.94 (d, J=7.2 Hz, 3H).

### Example 73-5:6-(((butylsulfinyl)methyl)thio)-4-(5-methylthiazol-4-yl)-[2,3′-bipyridine]-5-carbonitrile

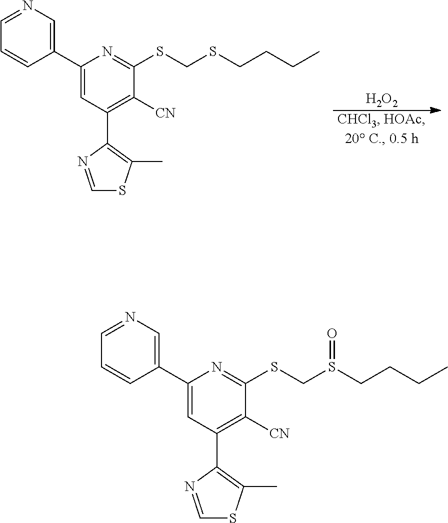 [SMILES: CCCCSCSc1nc(-c2cccnc2)cc(-c2ncsc2C)c1C#N ; "H2O2" ; "CHCl3, HOAc,20° C., 0.5 h" ; CCCCS(=O)CSc1nc(-c2cccnc2)cc(-c2ncsc2C)c1C#N]

Example 73-5 was prepared using General Procedure A, Step 5 starting from 6-(((butylthio)methyl)thio)-4-(5-methylthiazol-4-yl)-[2,3′-bipyridine]-5-carbonitrile (265 mg, 642.28 umol, 1 eq) and hydrogen peroxide (145.65 mg, 1.28 mmol, 123.43 μL, 30% purity, 2 eq) to give the target compound (240 mg, 87.19% yield) as a white solid.

### Example 73: 2-(butylsulfinyl)-4-(5-methylthiazol-4-yl)-6-(pyridin-3-yl)thieno[2,3-b]pyridin-3-amine

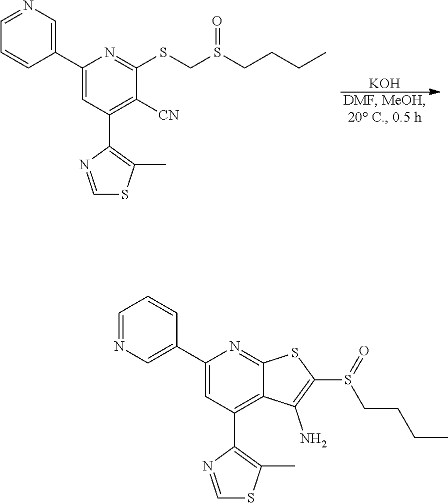 [SMILES: CCCCS(=O)CSc1nc(-c2cccnc2)cc(-c2ncsc2C)c1C#N ; "KOH" ; "DMF, MeOH,20° C., 0.5 h" ; CCCCS(=O)c1sc2nc(-c3cccnc3)cc(-c3ncsc3C)c2c1N]

Example 73 was prepared using General Procedure A, Step 6 starting from 6-(((butylsulfinyl)methyl)thio)-4-(5-methylthiazol-4-yl)-[2,3′-bipyridine]-5-carbonitrile (230 mg, 536.64 umol, 1 eq) to give the target compound (104 mg, 45% yield) as a yellow solid. LCMS: (ES⁺) m/z (M+H)⁺=429.1.

¹H NMR (400 MHz, CDCl₃) δ 9.13 (d, J=2.0 Hz, 1H), 8.71 (s, 1H), 8.57 (dd, J₁=1.6 Hz, J₂=4.8 Hz, 1H), 8.32-8.35 (m, 1H), 7.51 (s, 1H), 7.31-7.34 (dd, J₁=7.6 Hz, J₂=4.8 Hz, 1H), 5.15 (s, 2H), 3.13-3.18 (m, 1H), 2.96-3.03 (m, 1H), 2.45 (s, 3H), 1.55-1.66 (m, 2H), 1.32-1.42 (m, 2H), 0.82 (d, J=7.2 Hz, 3H).

### Example 74-1: 6-oxo-4-phenyl-2-(pyridin-3-yl)-1,6-dihydropyrimidine-5-carbonitrile

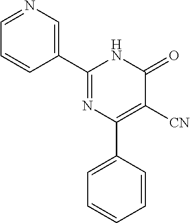 [SMILES: N#Cc1c(-c2ccccc2)nc(-c2cccnc2)[nH]c1=O]

A solution of benzaldehyde (1.00 g, 9.4 mmol), ethyl cyanoacetate (1064 mg, 9.4 mmol) and a catalytic amount of piperidine in MeOH (15 mL) was stirred at room temperature overnight. The solvent was evaporated to give the desired product in quantitative yield. The crude product (3.0 mmol, 603 mg) was dissolved in EtOH (8 mL) and nicotinimidamide hydrochloride (1.5 equiv, 4.5 mmol, 706 mg) and potassium carbonate (3.0 equiv, 9 mmol, 1.24 g) were added and the reaction mixture was stirred at 80° C. overnight. Once completed, the reaction mixture was filtered, the obtained solid suspended in water, filtered and dried to give 6-oxo-4-phenyl-2-(pyridin-3-yl)-1,6-dihydropyrimidine-5-carbonitrile (260 mg). ESI-MS (m/z): 275.1 [M+H]⁺.

### Example 74-2: 4-chloro-6-phenyl-2-(pyridin-3-yl)pyrimidine-5-carbonitrile

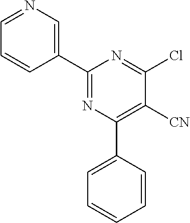 [SMILES: N#Cc1c(Cl)nc(-c2cccnc2)nc1-c1ccccc1]

The reaction mixture of 6-oxo-4-phenyl-2-(pyridin-3-yl)-1,6-dihydropyrimidine-5-carbonitrile in POCl₃ (1 mL) was stirred at 100° C. for 20 min. Once completed (the reaction progress was monitored by LCMS) the reaction mixture was cooled to room temperature and Et₂O was added. The formed solid/oil was separated from the liquid, dried and used in the next step without further purification. EST-MS (m/z): 293.1 [M+H]⁺.

### Example 74-3: 4-(((butylthio)methyl)thio)-6-phenyl-2-(pyridin-3-yl)pyrimidine-5-carbonitrile

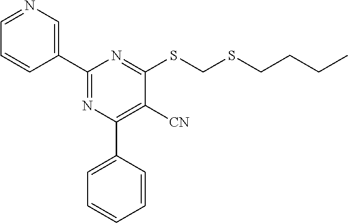 [SMILES: CCCCSCSc1nc(-c2cccnc2)nc(-c2ccccc2)c1C#N]

To the crude 4-(((butylthio)methyl)thio)-6-phenyl-2-(pyridin-3-yl)pyrimidine-5-carbonitrile (300 mg, 1.0 mmol) in DMF (1 mL) was added sodium sulfide (85 mg, 1.09 mmol, 1.09 equiv) and the reaction mixture was stirred at 80° C. for 20 min. The progress of the reaction was followed by LCMS. Once complete, conc. HCl was added and the reaction mixture was stirred in the hood for 10 min. ESI-MS (m/z): 291.0 [M+H]⁺.

The reaction mixture was diluted with CH₃CN (3 mL) and Et₃N (500 mg, 4.95 mmol) was added followed by butyl(chloromethyl)sulfane (3.0 mmol, 414 mg). The reaction mixture was stirred at room temperature for 15 min. Once complete, the reaction was diluted with EtOAc and water. The organic phase was separated and the aqueous layer was extracted twice with EtOAc. The combined extractions were washed with saturated NaCl solution, dried over magnesium sulfate, filtered and concentrated under reduced pressure. The residue was purified by flash chromatography to give 243 mg of product (62%). ¹H NMR (400 MHz, Chloroform-d) δ 9.83 (s, 1H), 9.43 (d, J=8.8 Hz, 1H), 9.03 (s, 1H), 8.28-8.02 (m, 3H), 7.75-7.47 (m, 3H), 4.53 (s, 2H), 2.76 (t, J=7.4 Hz, 2H), 1.64 (p, J=7.4 Hz, 2H), 1.43 (h, J=7.3 Hz, 2H), 0.92 (t, J=7.4 Hz, 3H). ESI-MS (m/z): 393.1 [M+H]⁺.

### Example 74-4: 4-(((butylsulfinyl)methyl)thio)-6-phenyl-2-(pyridin-3-yl)pyrimidine-5-carbonitrile

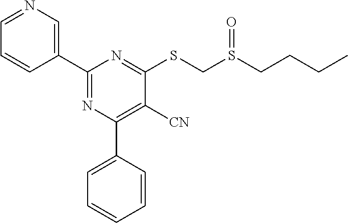 [SMILES: CCCCS(=O)CSc1nc(-c2cccnc2)nc(-c2ccccc2)c1C#N]

To the solution of 4-(((butylthio)methyl)thio)-6-phenyl-2-(pyridin-3-yl)pyrimidine-5-carbonitrile (53 mg, 0.13 mmol) in CHCl₃ (250 μL) and AcOH (250 μL) was added H₂O₂ (30 μL, 30% solution in water). The reaction mixture was stirred at 32° C. for 40 min. Once complete, the reaction was diluted with EtOAc and was washed with saturated NaHCO₃ solution, dried over magnesium sulfate, filtered and concentrated under reduce pressure to give 55 mg of desired compound. ¹H NMR (400 MHz, Chloroform-d) δ 9.76 (s, 1H), 8.86 (d, J=8.1 Hz, 1H), 8.82 (d, J=6.9 Hz, 1H), 8.19-8.12 (m, 2H), 7.68-7.56 (m, 4H), 4.77 (d, J=13.2 Hz, 1H), 4.59 (d, J=13.3 Hz, 1H), 3.05-2.85 (m, 2H), 1.95-1.74 (m, 2H), 1.64-1.43 (m, 2H), 0.97 (t, J=7.3 Hz, 3H). ESI-MS (m/z): 409.1 [M+H]⁺.

### Example 74: 6-(butylsulfinyl)-4-phenyl-2-(pyridin-3-yl)thieno[2,3-d]pyrimidin-5-amine

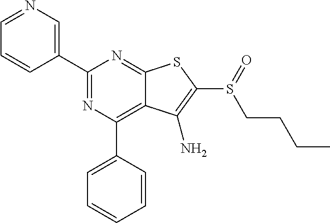 [SMILES: CCCCS(=O)c1sc2nc(-c3cccnc3)nc(-c3ccccc3)c2c1N]

To a solution of 6-(butylsulfinyl)-4-phenyl-2-(pyridin-3-yl)thieno[2,3-d]pyrimidin-5-amine (0.47 mmol, 193 mg) in DMF (3.0 mL) was added KOH (0.35 mmol, 20 mg, 0.75 equiv. in 200 μl of water). The reaction mixture was stirred at r.t. for 20 min (the reaction was monitored by TLC). Once complete, the reaction was diluted with EtOAc and washed with 5% aq. solution of acetic acid. The organic phase was separated and the aqueous layer was extracted twice with EtOAc, dried over magnesium sulfate, filtered and concentrated under reduced pressure to give crude product, which was purified by flash chromatography in 48% isolated yield. ¹H NMR (400 MHz, Methylene Chloride-d₂) δ 9.72 (s, 1H), 8.92-8.77 (m, 1H), 8.71 (d, J=4.7 Hz, 1H), 7.85-7.71 (m, 2H), 7.71-7.55 (m, 3H), 7.45 (dd, J=8.0, 4.8 Hz, 1H), 4.83 (s, 2H), 3.28 (ddd, J=12.7, 9.0, 6.1 Hz, 1H), 3.13 (ddd, J=12.8, 9.1, 6.6 Hz, 1H), 1.85-1.59 (m, 2H), 1.59-1.38 (m, 2H), 0.97 (t, J=7.6 Hz, 3H). ESI-MS (m/z): 409.1 [M+H]⁺.

Enantiomers were separated on a 1 cm Chiralpak AD-H column using 75% EtOH and 25% Hexanes with 5 mL/min flow rate, 400 μL injection (concentration 10 mg/ml) the 1st peak (Example 74-A/B) was at 5.5 min and the 2nd peak (Example 74-A/B) was at 14.7 min.

### Example 75-1: 3-chloro-5-(1-methyl-1H-pyrazol-4-yl)pyrazine-2-carbonitrile

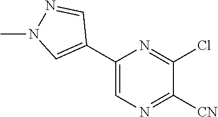

A mixture of 3,5-dichloropyrazine-2-carbonitrile (150 mg, 0.87 mmol), 1-methyl-4-(4,4,5,5-tetramethyl-1,3,2-dioxaborolan-2-yl)-1H-pyrazole (180 mg 0.87 mmol, 1.0 equiv), [1,1′-bis(diphenylphosphino)ferrocene]dichloropalladium(II) (0.04 mmol, 5 mol %) in degassed THF (1.7 mL) and aq. solution of sodium carbonate (2 M, 850 μL) was stirred for 4 h under nitrogen at 100° C. Once complete, the reaction was diluted with EtOAc and water. The organic phase was separated and the aqueous layer was extracted twice with EtOAc, dried over magnesium sulfate, filtered and concentrated under reduced pressure to give product: ESI-MS (m/z): 220.1

### Example 75-2: 3-(((butylthio)methyl)thio)-5-(1-methyl-1H-pyrazol-4-yl)pyrazine-2-carbonitrile

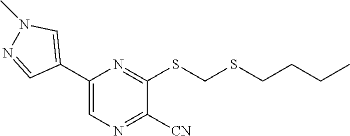 [SMILES: CCCCSCSc1nc(-c2cnn(C)c2)cnc1C#N]

To the crude 3-chloro-5-(1-methyl-1H-pyrazol-4-yl)pyrazine-2-carbonitrile (150 mg), in DMF (500 μL) was added sodium sulfide (53 mg, 0.685 mmol,) and the reaction mixture was stirred at 80° C. for 20 min. The progress of the reaction was followed by LCMS. Once complete, conc. HCl was added and the reaction mixture was stirred in the hood for 10 min.

The solution of crude 5-(1-methyl-1H-pyrazol-4-yl)-3-thioxo-3,4-dihydropyrazine-2-carbonitrile was diluted with CH₃CN (2 mL) and Et₃N (264 mg, 2.61 mmol) was added followed by butyl(chloromethyl)sulfane (1.37 mmol, 189 mg). The reaction mixture was stirred at 80° C. for 30 min. Once complete, the reaction was diluted with EtOAc and water. The organic phase was separated and aqueous layer was extracted twice with EtOAc. The combined extractions were washed with saturated NaCl solution, dried over magnesium sulfate, filtered and concentrated under reduced pressure. The residue was purified by flash chromatography to give 95 mg of product (34% isolated yield starting from cross coupling reaction of 3,5-dichloropyrazine-2-carbonitrile). ¹H NMR (400 MHz, Chloroform-d) δ 8.46 (s, 1H), 8.06 (s, 1H), 8.03 (s, 1H), 4.42 (s, 2H), 4.01 (s, 3H), 2.73 (t, J=7.3 Hz, 2H), 1.72-1.56 (m, 2H), 1.48-1.35 (m, 2H), 0.92 (t, J=7.3 Hz, 3H). ESI-MS (m/z): 320.1.

### Example 75-3: 3-(((butylsulfinyl)methyl)thio)-5-(1-methyl-1H-pyrazol-4-yl)pyrazine-2-carbonitrile

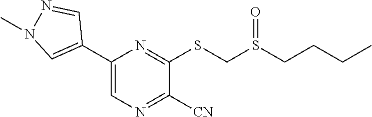 [SMILES: CCCCS(=O)CSc1nc(-c2cnn(C)c2)cnc1C#N]

The target compound was prepared in 99% isolated yield using the synthetic procedure described for the preparation of Example 74-4. ¹H NMR (400 MHz, Chloroform-d) δ 8.56 (s, 1H), 8.26 (s, 1H), 8.06 (s, 1H), 4.66 (d, J=13.1 Hz, 1H), 4.27 (d, J=13.1 Hz, 1H), 4.01 (s, 3H), 3.02-2.86 (m, 2H), 2.86-2.75 (m, 2H), 1.82 (t, J=6.8 Hz, 2H), 1.61-1.41 (m, 2H), 0.96 (t, J=7.3 Hz, 3H). ESI-MS (m/z): 336.1

### Example 75: 6-(butylsulfinyl)-3-(1-methyl-1H-pyrazol-4-yl)thieno[2,3-b]pyrazin-7-amine

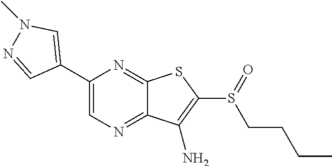 [SMILES: CCCCS(=O)c1sc2nc(-c3cnn(C)c3)cnc2c1N]

The target compound was prepared in 89% isolated yield, using synthetic procedures described for the preparation of Example 74, except using 1:1 DMF/MeOH solvent. ¹H NMR (400 MHz, Acetone-d₆) δ 8.96 (s, 1H), 8.35 (s, 1H), 8.13 (s, 1H), 5.73 (s, 2H), 3.99 (s, 3H), 3.28-3.00 (m, 2H), 1.93-1.65 (m, 2H), 1.65-1.38 (m, 2H), 0.95 (t, J=7.3 Hz, 3H). ESI-MS (m/z): 336.1.

### Example 76-1: 6-oxo-4-phenyl-2-(pyridin-2-yl)-1,6-dihydropyrimidine-5-carbonitrile

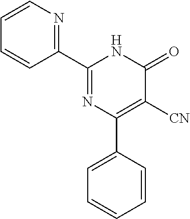 [SMILES: N#Cc1c(-c2ccccc2)nc(-c2ccccn2)[nH]c1=O]

The target compound was prepared from benzaldehyde, 2-cyanoethanethioamide and picolinimidamide hydrochloride using synthetic procedures described for the preparation of Example 74-1. ESI-MS (m/z): 275.1 [M+H]⁺.

### Example 76-2: 4-chloro-6-phenyl-2-(pyridin-2-yl)pyrimidine-5-carbonitrile

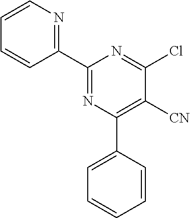 [SMILES: N#Cc1c(Cl)nc(-c2ccccn2)nc1-c1ccccc1]

The target compound was prepare using synthetic procedures described for the preparation of Example 74-2. ESI-MS (m/z): 293.1 [M+H]⁺.

### Example 76-3: 4-(((butylthio)methyl)thio)-6-phenyl-2-(pyridin-2-yl)pyrimidine-5-carbonitrile

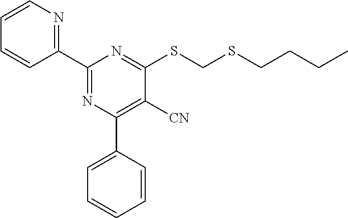 [SMILES: CCCCSCSc1nc(-c2ccccn2)nc(-c2ccccc2)c1C#N]

The target compound was prepared in 41% using synthetic procedures described for the preparation of Example 74, except that the alkylation reaction should be run at room temperature. Elevated temperature decreased the reaction yield. ¹H NMR (400 MHz, Chloroform-d) δ 8.88 (dd, J=4.7, 1.8, 1H), 8.57 (d, J=8.0, 1H), 8.27-8.02 (m, 2H), 7.89 (td, J=7.8, 1.7 Hz, 1H), 7.65-7.50 (m, 3H), 7.45 (dd, J=7.7, 4.8 Hz, 1H), 4.66 (s, 2H), 2.78 (t, J=7.4 Hz, 2H), 1.80-1.61 (m, 2H), 1.43 (h, J=7.4 Hz, 2H), 0.91 (t, J=7.5 Hz, 3H). ESI-MS (m/z): 393.1 [M+H]⁺.

### Example 76-4: 4-(((butylsulfinyl)methyl)thio)-6-phenyl-2-(pyridin-2-yl)pyrimidine-5-carbonitrile

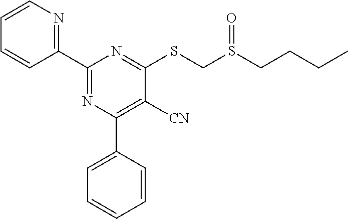 [SMILES: CCCCS(=O)CSc1nc(-c2ccccn2)nc(-c2ccccc2)c1C#N]

The target compound was prepared in 99% isolated yield, using synthetic procedures described for the preparation of Example 74. ¹H NMR (400 MHz, Chloroform-d) δ 8.89 (d, J=5.0 Hz, 1H), 8.63 (d, J=7.9 Hz, 1H), 8.19-8.10 (m, 2H), 7.95 (td, J=7.8, 1.7 Hz, 1H), 7.66-7.54 (m, 3H), 7.54-7.46 (m, 1H), 4.81 (d, J=13.3 Hz, 1H), 4.76 (d, J=13.3 Hz, 1H), 3.05 (dt, J=12.9, 8.1 Hz, 1H), 2.95 (dt, J=12.9, 7.2 Hz, 1H), 1.91-1.74 (m, 2H), 1.66-1.34 (m, 2H), 0.94 (t, J=7.3 Hz, 3H). ESI-MS (m/z): 409.1 [M+H]⁺.

### Example 76: 6-(butylsulfinyl)-4-phenyl-2-(pyridin-2-yl)thieno[2,3-d]pyrimidin-5-amine

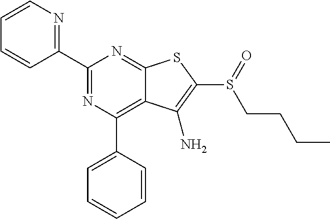 [SMILES: CCCCS(=O)c1sc2nc(-c3ccccn3)nc(-c3ccccc3)c2c1N]

The target compound was prepared in 66% isolated yield, using synthetic procedures described for the preparation of Example 74. ¹H NMR (400 MHz, Methylene Chloride-d₂) δ 8.83 (d, J=4.7 Hz, 1H), 8.62 (d, J=8.0 Hz, 1H), 7.89 (t, J=7.9 Hz, 1H), 7.78-7.70 (m, 2H), 7.68-7.58 (m, 3H), 7.49-7.39 (m, 1H), 4.83 (s, 2H), 3.36-3.23 (m, 1H), 3.23-3.09 (m, 1H), 1.90-1.68 (m, 2H), 1.62-1.42 (m, 2H), 0.99 (t, J=7.3 Hz, 3H). ESI-MS (m/z): 409.1 [M+H]⁺.

### Example 77-1: 4-(1-methyl-1H-imidazol-2-yl)-6-oxo-2-(thiazol-2-yl)-1,6-dihydropyrimidine-5-carbonitrile

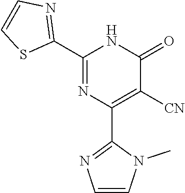 [SMILES: Cn1ccnc1-c1nc(-c2nccs2)[nH]c(=O)c1C#N]

The target compound was prepared from 1-methyl-1H-imidazole-2-carbaldehyde, 2-cyanoethanethioamide and thiazole-2-carboximidamide hydrochloride using synthetic procedures described for the preparation of Example 74-1. ESI-MS (m/z): 285.1 [M+H]⁺.

### Example 77-2: 4-chloro-6-(1-methyl-1H-imidazol-2-yl)-2-(thiazol-2-yl)pyrimidine-5-carbonitrile

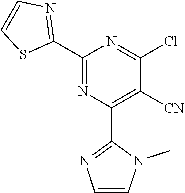 [SMILES: Cn1ccnc1-c1nc(-c2nccs2)nc(Cl)c1C#N]

The target compound was prepared using synthetic procedures described for the preparation of Example 74-2. ESI-MS (m/z): 303.1 [M+H]⁺.

### Example 77-3: 4-(((butylthio)methyl)thio)-6-(1-methyl-1H-imidazol-2-yl)-2-(thiazol-2-yl)pyrimidine-5-carbonitrile

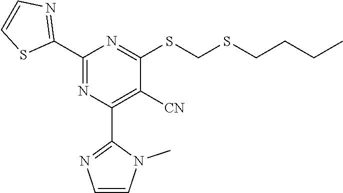 [SMILES: CCCCSCSc1nc(-c2nccs2)nc(-c2nccn2C)c1C#N]

The target compound was prepared in 5% isolated yield, using synthetic procedures described for the preparation of Example 74-3. ¹H NMR (400 MHz, Chloroform-d) δ 8.13 (d, J=3.1 Hz, 1H), 7.66 (d, J=3.1 Hz, 1H), 7.36 (s, 1H), 7.14 (s, 1H), 4.57 (s, 2H), 4.25 (s, 3H), 2.75 (t, J=7.4 Hz, 2H), 1.77-1.56 (m, 2H), 1.51-1.34 (m, 2H), 0.91 (t, J=7.6 Hz, 3H). ESI-MS (m/z): 403.1 [M+H]⁺.

### Example 77-4: 4-(((butylsulfinyl)methyl)thio)-6-(1-methyl-1H-imidazol-2-yl)-2-(thiazol-2-yl)pyrimidine-5-carbonitrile

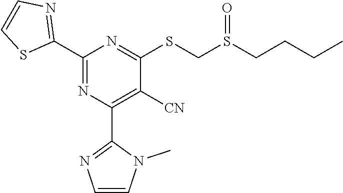 [SMILES: CCCCS(=O)CSc1nc(-c2nccs2)nc(-c2nccn2C)c1C#N]

The target compound was prepared in 96% yield using synthetic procedures described for the preparation of Example 74-4. ¹H NMR (400 MHz, Chloroform-d) δ 8.12 (d, J=3.1 Hz, 1H), 7.69 (d, J=3.1 Hz, 1H), 7.37 (s, 1H), 7.17 (s, 1H), 4.68 (d, J=13.3 Hz, 1H), 4.63 (d, J=13.2 Hz, 1H), 4.25 (s, 3H), 3.05 (dt, J=12.9, 8.1 Hz, 1H), 2.91 (dt, J=12.9, 7.0 Hz, 1H), 1.89-1.74 (m, 2H), 1.63-1.33 (m, 2H), 0.95 (t, J=7.4 Hz, 3H). ESI-MS (m/z): 419.1 [M+H]⁺.

### Example 77: 6-(butylsulfinyl)-4-(1-methyl-1H-imidazol-2-yl)-2-(thiazol-2-yl)thieno[2,3-d]pyrimidin-5-amine

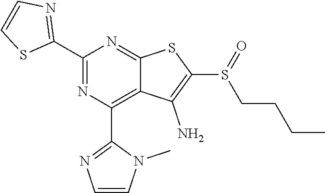 [SMILES: CCCCS(=O)c1sc2nc(-c3nccs3)nc(-c3nccn3C)c2c1N]

The target compound was prepared in 38% isolated yield, using synthetic procedures described for the preparation of Example 74. ¹H NMR (400 MHz, Methylene Chloride-d₂) δ 8.08 (d, J=3.1 Hz, 1H), 7.63 (d, J=3.1 Hz, 1H), 7.54 (s, 2H), 7.29 (d, J=1.0 Hz, 1H), 7.24 (d, J=1.0 Hz, 1H), 4.27 (s, 3H), 3.28 (ddd, J=12.7, 9.3, 5.8 Hz, 1H), 3.12 (ddd, J=12.8, 9.3, 6.3 Hz, 1H), 1.85-1.61 (m, 2H), 1.57-1.41 (m, 2H), 0.95 (t, J=7.3 Hz, 3H). ESI-MS (m/z): 419.1 [M+H]⁺.

### Example 78-1: 4-((((3-methoxypropyl)thio)methyl)thio)-6-phenyl-2-(pyridin-3-yl)pyrimidine-5-carbonitrile

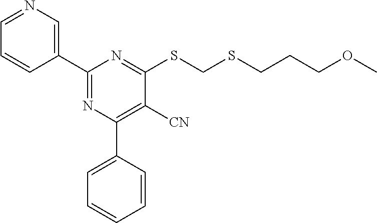 [SMILES: COCCCSCSc1nc(-c2cccnc2)nc(-c2ccccc2)c1C#N]

The target compound was prepared in 21% using synthetic procedures described for the preparation of Example 74. ¹H NMR (500 MHz, Chloroform-d) δ 9.79 (dd, J=2.2, 0.9 Hz, 1H), 8.99-8.63 (m, 2H), 8.30-8.02 (m, 2H), 7.76-7.56 (m, 3H), 7.51 (ddd, J=8.0, 4.9, 0.9 Hz, 1H), 4.61 (s, 2H), 3.52 (t, J=6.0 Hz, 2H), 3.36 (s, 3H), 2.88 (t, J=7.2 Hz, 2H), 2.05-1.87 (m, 2H). EST-MS (m/z): 409.1 [M+H]⁺.

### Example 78-2: 4-((((3-methoxypropyl)sulfinyl)methyl)thio)-6-phenyl-2-(pyridin-3-yl)pyrimidine-5-carbonitrile

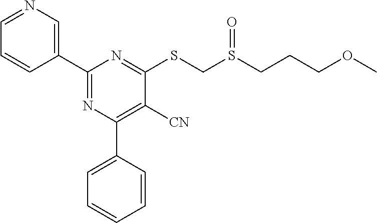 [SMILES: COCCCS(=O)CSc1nc(-c2cccnc2)nc(-c2ccccc2)c1C#N]

The target compound was prepared in 99% isolated yield, using synthetic procedures described for the preparation of the analog Example 74-3. ESI-MS (m/z): 425.1 [M+H]⁺.

### Example 78: 6-((3-methoxypropyl)sulfinyl)-4-phenyl-2-(pyridin-3-yl)thieno[2,3-d]pyrimidin-5-amine

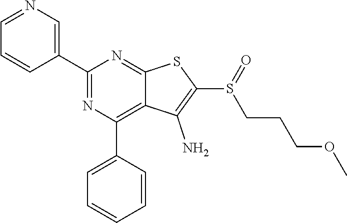 [SMILES: COCCCS(=O)c1sc2nc(-c3cccnc3)nc(-c3ccccc3)c2c1N]

The target compound was prepared using synthetic procedures described for the preparation of Example 74. ¹H NMR (400 MHz, Methylene Chloride-d₂) δ 9.72 (dd, J=2.0, 1.0 Hz, 1H), 8.89-8.77 (m, 1H), 8.71 (dd, J=4.7, 1.7 Hz, 1H), 7.83-7.70 (m, 2H), 7.70-7.57 (m, 3H), 7.45 (ddd, J=8.0, 4.8, 1.0 Hz, 1H), 4.83 (s, 2H), 3.50 (t, J=5.9 Hz, 2H), 3.38-3.28 (m, 1H), 3.32 (s, 3H), 3.22 (ddd, J=12.8, 8.1, 6.4 Hz, 1H), 2.09-1.89 (m, 2H). ESI-MS (m/z): 425.1 [M+H]⁺. Enantiomers were separated on a 1 cm Chiralpak AD-H column using 100% EtOH with 5 mL/min flow rate, 500 μL injection (concentration 10 mg/ml) the 1st peak (Example 78-B) was at 8.3 min and the 2nd peak (Example 78-A) was at 27.5 min.

### Example 79-1: 4-((((2-methoxyethyl)thio)methyl)thio)-6-phenyl-2-(pyridin-3-yl)pyrimidine-5-carbonitrile

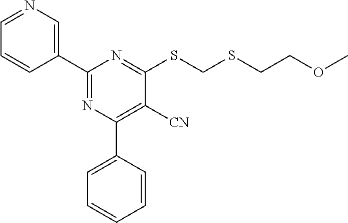 [SMILES: COCCSCSc1nc(-c2cccnc2)nc(-c2ccccc2)c1C#N]

The target compound was prepared in 23% using synthetic procedures described for the preparation of Example 74. EST-MS (m/z): 395.1 [M+H]⁺.

### Example 79-2: 4-((((3-methoxyethyl)sulfinyl)methyl)thio)-6-phenyl-2-(pyridin-3-yl)pyrimidine-5-carbonitrile

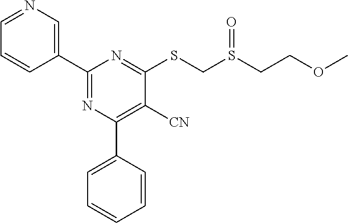 [SMILES: COCCS(=O)CSc1nc(-c2cccnc2)nc(-c2ccccc2)c1C#N]

The target compound was prepared in 99% isolated yield, using synthetic procedures described for the preparation of Example 74-4. ESI-MS (m/z): 411.1 [M+H]⁺.

### Example 79: 6-((3-methoxyethyl)sulfinyl)-4-phenyl-2-(pyridin-3-yl)thieno[2,3-d]pyrimidin-5-amine

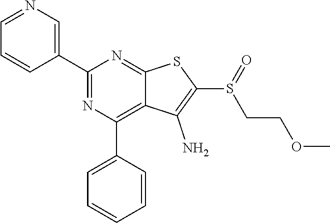 [SMILES: COCCS(=O)c1sc2nc(-c3cccnc3)nc(-c3ccccc3)c2c1N]

The target compound was prepared in 35% isolated yield, using synthetic procedures described for the preparation of Example 74. ¹H NMR (400 MHz, Methylene Chloride-d₂) δ 9.73 (s, 1H), 8.82 (dt, J=8.0, 2.1 Hz, 1H), 8.71 (dd, J=4.8, 1.8 Hz, 1H), 7.80-7.70 (m, 2H), 7.70-7.57 (m, 3H), 7.45 (ddt, J=8.0, 4.8, 0.8 Hz, 1H), 4.79 (s, 2H), 3.84 (ddd, J=10.3, 7.7, 4.0 Hz, 1H), 3.75-3.63 (m, 1H), 3.58 (ddd, J=12.8, 6.2, 3.9 Hz, 1H), 3.37 (s, 3H), 3.35-3.21 (m, 2H). ESI-MS (m/z): 411.1 [M+H]⁺. Enantiomers were separated on a 1 cm Chiralpak AD-H column using 80% EtOH and 20% Hexanes with 5 m/min flow rate, 400 μL injection (concentration 10 mg/ml) the 1st peak (Example 79-B) was at 8.2 min and the 2nd peak was at 12.8 min (Example 79-A).

### Example 80-1: 6-oxo-4-phenyl-1,6-dihydro-[2,2′-bipyrimidine]-5-carbonitrile

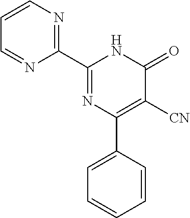 [SMILES: N#Cc1c(-c2ccccc2)nc(-c2ncccn2)[nH]c1=O]

The target compound was prepared from benzaldehyde, 2-cyanoethanethioamide and pyrimidine-2-carboximidamide hydrochloride using synthetic procedures described for the preparation of Example 74-1. ESI-MS (m/z): 276.1 [M+H]⁺.

### Example 80-2: 4-chloro-6-phenyl-[2,2′-bipyrimidine]-5-carbonitrile

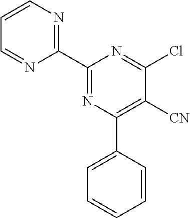 [SMILES: N#Cc1c(Cl)nc(-c2ncccn2)nc1-c1ccccc1]

The target compound was prepared using synthetic procedures described for the preparation of Example 74-2. ESI-MS (m/z): 294.1 [M+H]⁺.

### Example 80-3: 4-(((butylthio)methyl)thio)-6-phenyl-[2,2′-bipyrimidine]-5-carbonitrile

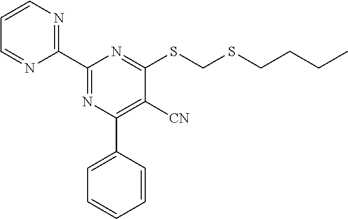 [SMILES: CCCCSCSc1nc(-c2ncccn2)nc(-c2ccccc2)c1C#N]

The target compound was prepared using synthetic procedures described for the preparation of Example 74-3. ESI-MS (m/z): 394.1 [M+H]⁺.

### Example 80-4: 4-(((butylsulfinyl)methyl)thio)-6-phenyl-[2,2′-bipyrimidine]-5-carbonitrile

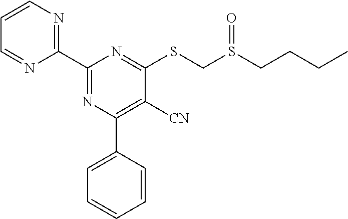 [SMILES: CCCCS(=O)CSc1nc(-c2ncccn2)nc(-c2ccccc2)c1C#N]

The target compound was prepared using synthetic procedures described for the preparation of Example 74. ESI-MS (m/z): 410.1 [M+H]⁺.

### Example 80: 6-(butylsulfinyl)-4-phenyl-2-(pyrimidin-2-yl)thieno[2,3-d]pyrimidin-5-amine

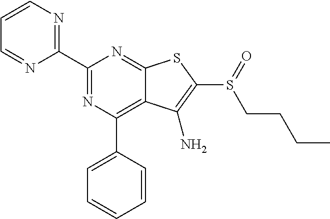 [SMILES: CCCCS(=O)c1sc2nc(-c3ncccn3)nc(-c3ccccc3)c2c1N]

The target compound was prepared in 65% isolated yield, using synthetic procedures described for the preparation of Example 74. ¹H NMR (400 MHz, Methylene Chloride-d₂) δ 9.01 (d, J=4.9 Hz, 2H), 7.74-7.68 (m, 2H), 7.68-7.58 (m, 3H), 7.46 (t, J=4.8 Hz, 1H), 4.85 (s, 2H), 3.30 (ddd, J=12.9, 8.8, 6.2 Hz, 1H), 3.14 (ddd, J=12.8, 8.9, 7.1 Hz, 1H), 1.83-1.63 (m, 2H), 1.63-1.42 (m, 2H), 0.96 (d, J=7.7 Hz, 3H). ESI-MS (m/z): 410.1 [M+H]⁺.

### Example 81-1: 6-oxo-2-(pyridin-3-yl)-4-(thiazol-4-yl)-1,6-dihydropyrimidine-5-carbonitrile

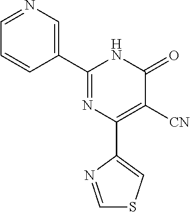 [SMILES: N#Cc1c(-c2cscn2)nc(-c2cccnc2)[nH]c1=O]

The target compound was prepared from thiazole-4-carbaldehyde, 2-cyanoethanethioamide and nicotinimidamide hydrochloride using synthetic procedures described for the preparation of Example 74-1. ESI-MS (m/z): 282.0 [M+H]⁺.

### Example 81-2: 4-chloro-2-(pyridin-3-yl)-6-(thiazol-4-yl)pyrimidine-5-carbonitrile

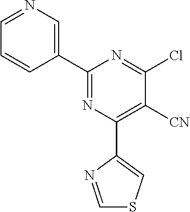 [SMILES: N#Cc1c(Cl)nc(-c2cccnc2)nc1-c1cscn1]

The target compound was prepared synthetic procedures described for the preparation of the Example 74-2. ESI-MS (m/z): 300.1 [M+H]⁺.

### Example 81-3: 4-(((butylthio)methyl)thio)-2-(pyridin-3-yl)-6-(thiazol-4-yl)pyrimidine-5-carbonitrile

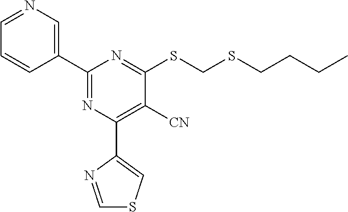 [SMILES: CCCCSCSc1nc(-c2cccnc2)nc(-c2cscn2)c1C#N]

The target compound was prepared in 21% isolated yield, using synthetic procedures described for the preparation of Example 74-3. ¹H NMR (400 MHz, Chloroform-d) δ 9.75 (d, J=2.2 Hz, 1H), 9.05 (d, J=2.1 Hz, 1H), 8.83-8.75 (m, 2H), 8.66 (d, J=2.2 Hz, 1H), 7.49 (ddd, J=8.0, 4.9, 0.9 Hz, 1H), 4.55 (s, 2H), 2.76 (t, J=7.2 Hz, 2H), 1.82-1.55 (m, 2H), 1.43 (h, J=7.3 Hz, 2H), 0.92 (t, J=7.3 Hz, 3H). ESI-MS (m/z): 400.1 [M+H]⁺.

### Example 81-4: 4-(((butylsulfinyl)methyl)thio)-2-(pyridin-3-yl)-6-(thiazol-4-yl)pyrimidine-5-carbonitrile

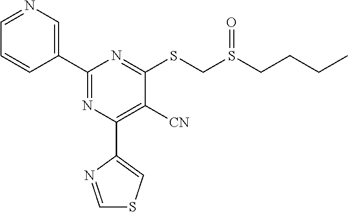 [SMILES: CCCCS(=O)CSc1nc(-c2cccnc2)nc(-c2cscn2)c1C#N]

The target compound was prepared in quantitative yield, using synthetic procedures described for the preparation of Example 74-4. ¹H NMR (400 MHz, Chloroform-d) δ 9.75 (s, 1H), 9.06 (d, J=2.1 Hz, 1H), 8.90-8.76 (m, 2H), 8.71 (d, J=2.1 Hz, 1H), 7.52 (dd, J=7.9, 5.0 Hz, 1H), 4.79 (d, J=13.2 Hz, 1H), 4.49 (d, J=13.2 Hz, 1H), 2.95 (dt, J=12.8, 8.1 Hz, 1H), 2.86 (dt, J=12.9, 7.1 Hz, 1H), 2.01-1.76 (m, 2H), 1.62-1.37 (m, 2H), 0.96 (t, J=7.3 Hz, 3H). ESI-MS (m/z): 416.1 [M+H]⁺.

### Example 81: 6-(butylsulfinyl)-2-(pyridin-3-yl)-4-(thiazol-4-yl)thieno[2,3-d]pyrimidin-5-amine

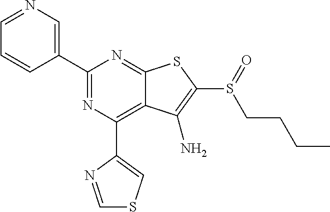 [SMILES: CCCCS(=O)c1sc2nc(-c3cccnc3)nc(-c3cscn3)c2c1N]

The target compound was prepared in 70% isolated yield, using synthetic procedures described for the preparation of Example 74. ¹H NMR (400 MHz, Methylene Chloride-d₂) δ 9.78 (s, 1H), 9.09 (d, J=2.2 Hz, 1H), 8.88 (d, J=2.3 Hz, 1H), 8.87-8.80 (m, 1H), 8.74 (s, 1H), 7.54-7.46 (m, 1H), 7.43 (s, 2H), 3.29 (ddd, J=12.7, 9.3, 5.8 Hz, 1H), 3.14 (ddd, J=12.7, 9.4, 6.3 Hz, 1H), 1.86-1.64 (m, 2H), 1.56-1.37 (m, 2H), 0.96 (t, J=7.3 Hz, 3H). ESI-MS (m/z): 416.1 [M+H]⁺.

### Example 82-1: 6-oxo-2-(pyridin-3-yl)-4-(thiazol-2-yl)-1,6-dihydropyrimidine-5-carbonitrile

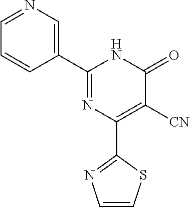 [SMILES: N#Cc1c(-c2nccs2)nc(-c2cccnc2)[nH]c1=O]

The target compound was prepared from thiazole-2-carbaldehyde, 2-cyanoethanethioamide and nicotinimidamide hydrochloride using synthetic procedures described for the preparation of Example 74-1. ESI-MS (m/z): 282.0 [M+H]⁺.

### Example 82-2: 4-chloro-2-(pyridin-3-yl)-6-(thiazol-2-yl)pyrimidine-5-carbonitrile

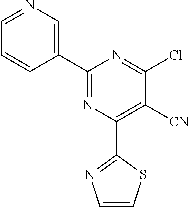 [SMILES: N#Cc1c(Cl)nc(-c2cccnc2)nc1-c1nccs1]

The target compound was prepared using synthetic procedures described for the preparation of Example 74-2. EST-MS (m/z): 300.1 [M+H]⁺.

### Example 82-3: 4-(((butylthio)methyl)thio)-2-(pyridin-3-yl)-6-(thiazol-2-yl)pyrimidine-5-carbonitrile

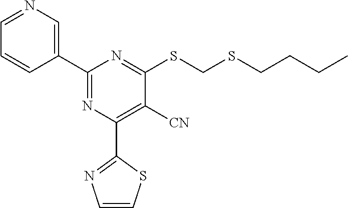 [SMILES: CCCCSCSc1nc(-c2cccnc2)nc(-c2nccs2)c1C#N]

The target compound was prepared in 20% isolated yield, using synthetic procedures described for the preparation of Example 74-3. ¹H NMR (400 MHz, Chloroform-d) δ 9.75 (s, 1H), 8.82 (s, 1H), 8.78 (d, J=8.1 Hz, 1H), 8.20 (d, J=3.1 Hz, 1H), 7.71 (d, J=3.1 Hz, 1H), 7.51 (dd, J=8.0, 4.7 Hz, 1H), 4.56 (s, 2H), 2.75 (t, J=7.4 Hz, 2H), 1.77-1.52 (m, 2H), 1.52-1.33 (m, 2H), 0.92 (t, J=7.3 Hz, 3H). ESI-MS (m/z): 400.1 [M+H]⁺.

### Example 82-4: 4-(((butylsulfinyl)methyl)thio)-2-(pyridin-3-yl)-6-(thiazol-2-yl)pyrimidine-5-carbonitrile

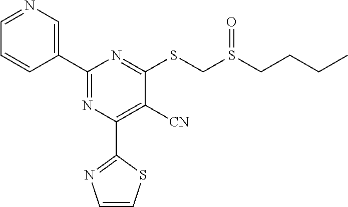 [SMILES: CCCCS(=O)CSc1nc(-c2cccnc2)nc(-c2nccs2)c1C#N]

The target compound was prepared in quantitative yield, using synthetic procedures described for the preparation of Example 74-4. ¹H NMR (400 MHz, Chloroform-d) δ 9.75 (s, 1H), 8.96-8.79 (m, 2H), 8.22 (d, J=3.1 Hz, 1H), 7.75 (d, J=3.1 Hz, 1H), 7.62-7.40 (m, 1H), 4.78 (d, J=13.2 Hz, 1H), 4.54 (d, J=13.2 Hz, 1H), 3.06-2.83 (m, 2H), 1.95-1.75 (m, 2H), 1.63-1.40 (m, 2H), 0.96 (t, J=7.3 Hz, 3H). ESI-MS (m/z): 416.1 [M+H]⁺.

### Example 82: 6-(butylsulfinyl)-2-(pyridin-3-yl)-4-(thiazol-2-yl)thieno[2,3-d]pyrimidin-5-amine

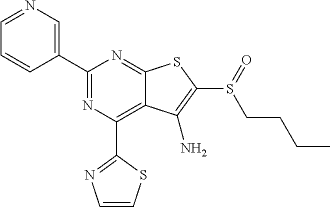 [SMILES: CCCCS(=O)c1sc2nc(-c3cccnc3)nc(-c3nccs3)c2c1N]

The target compound was prepared in 50% isolated yield, using synthetic procedures described for the preparation of Example 74. ¹H NMR (400 MHz, Methylene Chloride-d₂) δ 9.76 (s, 1H), 8.82 (dt, J=8.0, 2.0 Hz, 1H), 8.75 (dd, J=4.8, 1.7 Hz, 1H), 8.11 (d, J=3.2 Hz, 1H), 7.79-7.74 (m, 1H), 7.70 (s, 2H), 7.49 (ddd, J=7.8, 4.7, 0.9 Hz, 1H), 3.30 (ddd, J=12.7, 9.3, 6.3 Hz, 1H), 3.15 (ddd, J=12.7, 9.3, 6.3 Hz, 1H), 1.84-1.66 (m, 2H), 1.66-1.39 (m, 2H), 0.97 (t, J=7.3 Hz, 3H). EST-MS (m/z): 416.1 [M+H]⁺.

### Example 83-1: 6-oxo-4-phenyl-1,6-dihydro-[2,5′-bipyrimidine]-5-carbonitrile

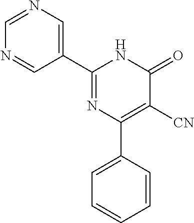 [SMILES: N#Cc1c(-c2ccccc2)nc(-c2cncnc2)[nH]c1=O]

The target compound was prepared from benzaldehyde, 2-cyanoethanethioamide and pyrimidine-5-carboximidamide hydrochloride using synthetic procedures described for the preparation of Example 74-1. ESI-MS (m/z): 276.1 [M+H]⁺.

### Example 83-2: 4-chloro-6-phenyl-[2,5′-bipyrimidine]-5-carbonitrile

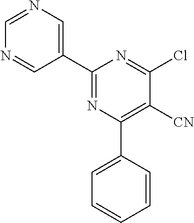 [SMILES: N#Cc1c(Cl)nc(-c2cncnc2)nc1-c1ccccc1]

The target compound was prepared synthetic procedures described for the preparation of the Example 74-2. ESI-MS (m/z): 294.1 [M+H]⁺.

### Example 83-3: 4-(((butylthio)methyl)thio)-6-phenyl-[2,5′-bipyrimidine]-5-carbonitrile

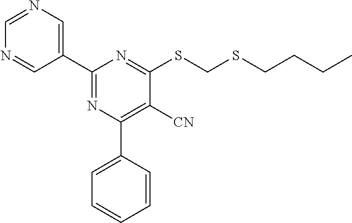 [SMILES: CCCCSCSc1nc(-c2cncnc2)nc(-c2ccccc2)c1C#N]

The target compound was prepared in 27% isolated yield, using synthetic procedures described for the preparation of Example 74-3. ¹H NMR (400 MHz, Chloroform-d) δ 9.79 (s, 2H), 9.39 (s, 1H), 8.21-8.05 (m, 2H), 7.73-7.49 (m, 3H), 4.55 (s, 2H), 2.77 (t, J=7.3 Hz, 2H), 1.72-1.59 (m, 2H), 1.59-1.36 (m, 2H), 0.93 (t, J=7.3 Hz, 3H). ESI-MS (m/z): 394.1 [M+H]⁺.

### Example 83-4: 4-(((butylsulfinyl)methyl)thio)-6-phenyl-[2,5′-bipyrimidine]-5-carbonitrile

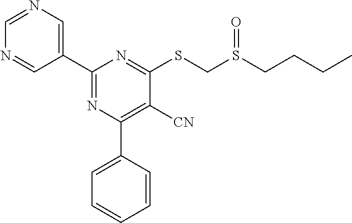 [SMILES: CCCCS(=O)CSc1nc(-c2cncnc2)nc(-c2ccccc2)c1C#N]

The target compound was prepared in quantitative yield, using synthetic procedures described for the preparation of Example 74-4. ¹H NMR (400 MHz, Chloroform-d) δ 9.79 (s, 2H), 9.40 (s, 1H), 8.28-8.05 (m, 2H), 7.80-7.53 (m, 3H), 4.68 (d, J=13.3 Hz, 1H), 4.64 (d, J=13.3 Hz, 1H), 3.17-2.58 (m, 2H), 2.00-1.70 (m, 2H), 1.70-1.39 (m, 2H), 0.98 (t, J=7.3 Hz, 3H). ESI-MS (m/z): 410.1 [M+H]⁺.

### Example 83: 6-(butylsulfinyl)-4-phenyl-2-(pyrimidin-5-yl)thieno[2,3-d]pyrimidin-5-amine

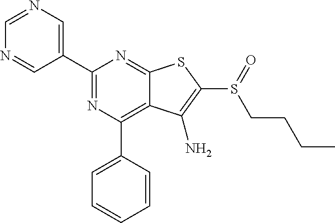 [SMILES: CCCCS(=O)c1sc2nc(-c3cncnc3)nc(-c3ccccc3)c2c1N]

The target compound was prepare in 54% isolated yield, using synthetic procedures described for the preparation of Example 74. ¹H NMR (400 MHz, Methylene Chloride-d₂) δ 9.78 (s, 2H), 9.30 (s, 1H), 7.81-7.70 (m, 2H), 7.70-7.54 (m, 3H), 4.86 (s, 2H), 3.29 (ddd, J=12.8, 8.6, 6.0 Hz, 1H), 3.14 (ddd, J=12.8, 9.0, 6.8 Hz, 1H), 1.90-1.66 (m, 2H), 1.51 (hd, J=7.3, 2.3 Hz, 2H), 0.97 (t, J=7.3 Hz, 3H). ESI-MS (m/z): 410.1 [M+H]⁺.

### Example 84-1: 4-(1-methyl-1H-imidazol-5-yl)-6-oxo-2-(pyridin-3-yl)-1,6-dihydropyrimidine-5-carbonitrile

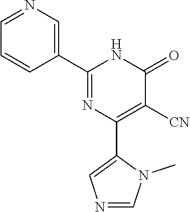 [SMILES: Cn1cncc1-c1nc(-c2cccnc2)[nH]c(=O)c1C#N]

The target compound was prepared from 1-methyl-1H-imidazole-5-carbaldehyde, 2-cyanoethanethioamide and nicotinimidamide hydrochloride using synthetic procedures described for the preparation of Example 74-1. ESI-MS (m/z): 279.1 [M+H]⁺.

### Example 84-2: 4-chloro-6-(1-methyl-1H-imidazol-5-yl)-2-(pyridin-3-yl)pyrimidine-5-carbonitrile

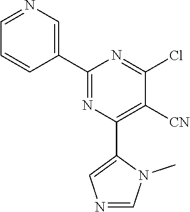 [SMILES: Cn1cncc1-c1nc(-c2cccnc2)nc(Cl)c1C#N]

The target compound was prepared synthetic procedures described for the preparation of Example 74-2. EST-MS (m/z): 297.1 [M+H]⁺.

### Example 84-3: 4-(((butylthio)methyl)thio)-6-(1-methyl-1H-imidazol-5-yl)-2-(pyridin-3-yl)pyrimidine-5-carbonitrile

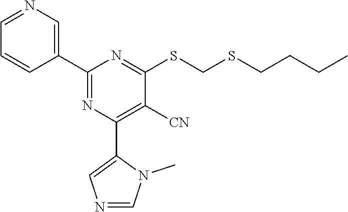 [SMILES: CCCCSCSc1nc(-c2cccnc2)nc(-c2cncn2C)c1C#N]

The target compound was prepared in 7% isolated yield, using synthetic procedures described for the preparation of Example 74-3. ¹H NMR (400 MHz, Methanol-d₄) δ 9.73 (s, 1H), 9.33-9.12 (m, 2H), 8.93 (d, J=5.1 Hz, 1H), 8.50 (s, 1H), 7.99 (dd, J=8.5, 5.5 Hz, 1H), 4.76 (s, 2H), 4.29 (s, 3H), 2.80 (t, J=7.3 Hz, 2H), 1.76-1.57 (m, 2H), 1.53-1.37 (m, 2H), 1.31 (t, J=7.3 Hz, 3H). ESI-MS (m/z): 397.1 [M+H]⁺.

### Example 84-4: 4-(((butylsulfinyl)methyl)thio)-6-(1-methyl-1H-imidazol-5-yl)-2-(pyridin-3-yl)pyrimidine-5-carbonitrile

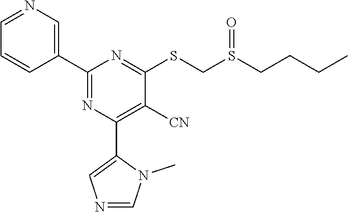 [SMILES: CCCCS(=O)CSc1nc(-c2cccnc2)nc(-c2cncn2C)c1C#N]

The target compound was prepared in 80% yield, using synthetic procedures described for the preparation of Example 74-4. ¹H NMR (400 MHz, Chloroform-d) δ 9.66 (dd, J=2.2, 0.9 Hz, 1H), 8.80 (dd, J=4.9, 1.7 Hz, 1H), 8.76 (dt, J=8.0, 2.0 Hz, 1H), 8.35 (d, J=0.8 Hz, 1H), 7.77 (s, 1H), 7.54 (ddd, J=8.0, 4.8, 0.9 Hz, 1H), 4.77 (d, J=13.3 Hz, 1H), 4.51 (d, J=13.2 Hz, 1H), 4.16 (s, 3H), 3.03-2.76 (m, 2H), 1.91-1.74 (m, 2H), 1.65-1.37 (m, 2H), 0.97 (t, J=7.3 Hz, 3H). ESI-MS (m/z): 413.1 [M+H]⁺.

### Example 84: 6-(butylsulfinyl)-4-(1-methyl-1H-imidazol-5-yl)-2-(pyridin-3-yl)thieno[2,3-d]pyrimidin-5-amine

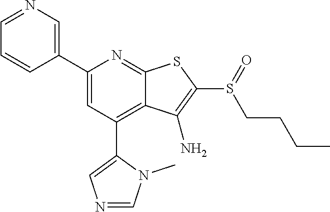 [SMILES: CCCCS(=O)c1sc2nc(-c3cccnc3)cc(-c3cncn3C)c2c1N]

The target compound was prepared in 44% isolated yield, using synthetic procedures described for the preparation of Example 74. ¹H NMR (400 MHz, Methylene Chloride-d₂) δ 9.75-9.64 (m, 1H), 8.79 (dt, J=8.0, 2.0 Hz, 1H), 8.73 (dd, J=4.8, 1.7 Hz, 1H), 7.76 (s, 1H), 7.56 (s, 1H), 7.48 (ddd, J=8.0, 4.8, 0.9 Hz, 1H), 5.31 (s, 2H), 4.00 (s, 3H), 3.31 (ddd, J=12.9, 9.1, 6.2 Hz, 2H), 3.16 (ddd, J=12.8, 9.0, 6.7 Hz, 1H), 1.87-1.66 (m, 2H), 1.02-0.92 (m, 2H), 0.98 (t, J=7.3 Hz, 3H). ESI-MS (m/z): 413.1 [M+H]⁺.

### Example 85-1: 4-(1-methyl-1H-imidazol-4-yl)-6-oxo-2-(pyridin-3-yl)-1,6-dihydropyrimidine-5-carbonitrile

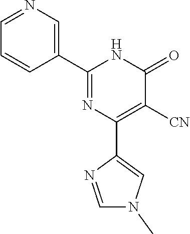 [SMILES: Cn1cnc(-c2nc(-c3cccnc3)[nH]c(=O)c2C#N)c1]

The target compound was prepared from 1-methyl-1H-imidazole-4-carbaldehyde, 2-cyanoethanethioamide and nicotinimidamide hydrochloride using synthetic procedures described for the preparation of Example 74-1. ESI-MS (m/z): 279.1 [M+H]⁺.

### Example 85-2: 4-chloro-6-(1-methyl-1H-imidazol-4-yl)-2-(pyridin-3-yl)pyrimidine-5-carbonitrile

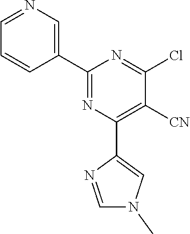 [SMILES: Cn1cnc(-c2nc(-c3cccnc3)nc(Cl)c2C#N)c1]

The target compound was prepared using synthetic procedures described for the preparation of Example 74-2. ESI-MS (m/z): 297.1 [M+H]⁺.

### Example 85-3: 4-(((butylthio)methyl)thio)-6-(1-methyl-1H-imidazol-4-yl)-2-(pyridin-3-yl)pyrimidine-5-carbonitrile

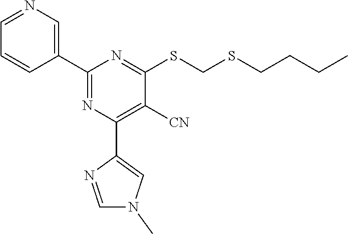 [SMILES: CCCCSCSc1nc(-c2cccnc2)nc(-c2cn(C)cn2)c1C#N]

The target compound was prepared in 8% isolated yield, using synthetic procedures described for the preparation of Example 74-3. ¹H NMR (500 MHz, Chloroform-d) δ 9.71 (s, 1H), 9.00 (d, J=7.3 Hz, 1H), 8.89 (s, 1H), 7.77 (d, J=7.1 Hz, 1H), 7.42 (s, 1H), 7.21 (s, 1H), 2.79 (t, J=7.4 Hz, 2H), 1.80-1.59 (m, 2H), 1.59-1.37 (m, 2H), 0.95 (t, J=7.5, 3H). EST-MS (m/z): 397.1 [M+H]⁺.

### Example 85-4: 4-(((butylsulfinyl)methyl)thio)-6-(1-methyl-1H-imidazol-4-yl)-2-(pyridin-3-yl)pyrimidine-5-carbonitrile

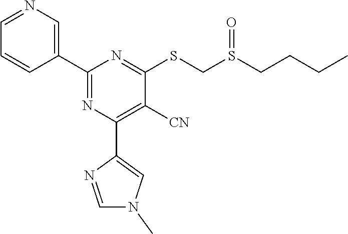 [SMILES: CCCCS(=O)CSc1nc(-c2cccnc2)nc(-c2cn(C)cn2)c1C#N]

The target compound was prepared using synthetic procedures described for the preparation of Example 74-4. ¹H NMR (400 MHz, Chloroform-d) δ 9.72 (s, 1H), 8.82 (dt, J=8.1, 2.0 Hz, 1H), 8.80-8.74 (m, 1H), 8.06 (s, 1H), 7.68 (s, 1H), 7.50 (dd, J=8.0, 4.6 Hz, 1H), 4.80 (d, J=13.2 Hz, 1H), 4.42 (d, J=13.1 Hz, 1H), 3.85 (s, 3H), 2.98-2.90 (m, 1H), 2.85 (dt, J=13.0, 7.3 Hz, 1H), 1.83 (p, J=7.7 Hz, 2H), 1.59-1.37 (m, 2H), 0.95 (t, J=7.3 Hz, 3H). ESI-MS (m/z): 413.1 [M+H]⁺.

### Example 85: 6-(butylsulfinyl)-4-(1-methyl-1H-imidazol-4-yl)-2-(pyridin-3-yl)thieno[2,3-d]pyrimidin-5-amine

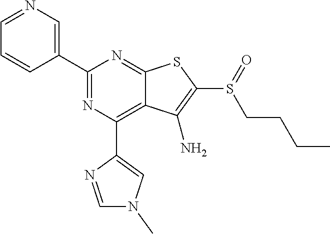 [SMILES: CCCCS(=O)c1sc2nc(-c3cccnc3)nc(-c3cn(C)cn3)c2c1N]

The target compound was prepared in 20% isolated yield, using synthetic procedures described for the preparation of Example 74. ¹H NMR (400 MHz, Methylene Chloride-d₂) δ 9.74 (s, 1H), 8.81 (dt, J=8.0, 1.9 Hz, 1H), 8.76-8.66 (m, 1H), 8.21 (s, 1H), 8.02 (s, 2H), 7.64 (s, 1H), 7.47 (dd, J=8.0, 4.8 Hz, 1H), 3.86 (s, 3H), 3.31-3.20 (m, 1H), 3.18-3.04 (m, 1H), 1.88-1.58 (m, 2H), 1.58-1.39 (m, 2H), 0.96 (td, J=7.3, 0.9 Hz, 3H). ESI-MS (m/z): 413.1 [M+H]⁺.

### Example 86-1: 4-(1-methyl-1H-imidazol-2-yl)-6-oxo-2-(pyridin-3-yl)-1,6-dihydropyrimidine-5-carbonitrile

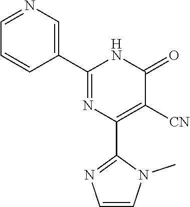 [SMILES: Cn1ccnc1-c1nc(-c2cccnc2)[nH]c(=O)c1C#N]

The target compound was prepared from 1-methyl-1H-imidazole-2-carbaldehyde, 2-cyanoethanethioamide and nicotinimidamide hydrochloride using synthetic procedures described for the preparation of Example 74-1. EST-MS (m/z): 279.1 [M+H]⁺.

### Example 86-2: 4-chloro-6-(1-methyl-1H-imidazol-2-yl)-2-(pyridin-3-yl)pyrimidine-5-carbonitrile

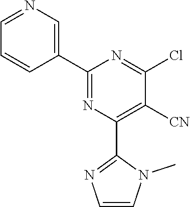 [SMILES: Cn1ccnc1-c1nc(-c2cccnc2)nc(Cl)c1C#N]

The target compound was prepared using synthetic procedures described for the preparation of Example 74-2. ESI-MS (m/z): 297.1 [M+H]⁺.

### Example 86-3: 4-(((butylthio)methyl)thio)-6-(1-methyl-1H-imidazol-2-yl)-2-(pyridin-3-yl)pyrimidine-5-carbonitrile

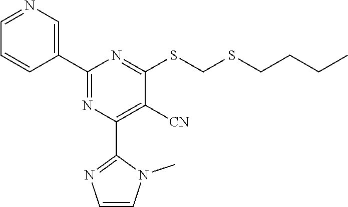 [SMILES: CCCCSCSc1nc(-c2cccnc2)nc(-c2nccn2C)c1C#N]

The target compound was prepared in 20% isolated yield, using synthetic procedures described for the preparation of Example 74-3. ¹H NMR (500 MHz, Chloroform-d) δ 9.71 (s, 1H), 9.00 (d, J=7.3 Hz, 1H), 8.89 (s, 1H), 7.77 (s, 1H), 7.42 (s, 1H), 7.21 (s, 1H), 4.55 (s, 2H), 4.26 (s, 2H) 2.79 (t, J=7.4 Hz, 2H), 1.80-1.59 (m, 2H), 1.59-1.37 (m, 2H), 0.95 (t, J=7.5, 3H). ESI-MS (m/z): 397.1 [M+H]⁺.

### Example 86-4: 4-(((butylsulfinyl)methyl)thio)-6-(1-methyl-1H-imidazol-2-yl)-2-(pyridin-3-yl)pyrimidine-5-carbonitrile

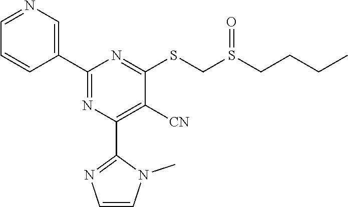 [SMILES: CCCCS(=O)CSc1nc(-c2cccnc2)nc(-c2nccn2C)c1C#N]

The target compound was prepared in 89% yield, using synthetic procedures described for the preparation of Example 74-4. ¹H NMR (400 MHz, Chloroform-d) δ 9.67 (s, 1H), 8.90-8.67 (m, 2H), 7.54 (dd, J=7.8, 5.1 Hz, 1H), 7.40 (s, 1H), 7.18 (s, 1H), 4.82 (d, J=13.2 Hz, 1H), 4.44 (d, J=13.2 Hz, 1H), 4.26 (s, 3H), 2.96 (dt, J=13.1, 8.1 Hz, 1H), 2.87 (dt, J=13.1, 7.3 Hz, 1H), 1.92-1.78 (m, 2H), 1.61-1.42 (m, 2H), 0.97 (t, J=7.2 Hz, 3H). EST-MS (m/z): 413.1 [M+H]⁺.

### Example 86: 6-(butylsulfinyl)-4-(1-methyl-1H-imidazol-2-yl)-2-(pyridin-3-yl)thieno[2,3-d]pyrimidin-5-amine

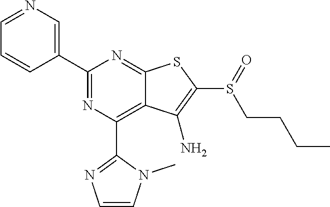 [SMILES: CCCCS(=O)c1sc2nc(-c3cccnc3)nc(-c3nccn3C)c2c1N]

The target compound was prepared in 61% isolated yield, using synthetic procedures described for the preparation of Example 74. ¹H NMR (400 MHz, Methylene Chloride-d₂) δ 9.68 (d, J=2.2 Hz, 1H), 8.77 (dt, J=8.0, 2.0 Hz, 1H), 8.73 (dd, J=4.8, 1.7 Hz, 1H), 7.48 (ddd, J=8.0, 4.7, 0.9 Hz, 1H), 7.43 (s, 2H), 7.29 (d, J=1.1 Hz, 1H), 7.25 (d, J=1.1 Hz, 1H), 4.24 (s, 3H), 3.27 (ddd, J=12.7, 9.3, 5.8 Hz, 1H), 3.12 (ddd, J=12.8, 9.4, 6.2 Hz, 1H), 1.84-1.57 (m, 2H), 1.57-1.42 (m, 2H), 0.95 (t, J=7.3 Hz, 3H). ESI-MS (m/z): 413.1 [M+H]⁺. Enantiomers were separated on a 1 cm Chiralpak AD-H column using 100% MeOH with 5 m/min flow rate, 400 μL injection (concentration 8 mg/ml) the 1st peak (Example 86B) was at 12.6 min and the 2nd peak (Example 86A) was at 43 min.

### Example 87-1: 4-(4-fluorophenyl)-6-((((3-methoxypropyl)thio)methyl)thio)-2-(pyridin-3-yl)pyrimidine-5-carbonitrile

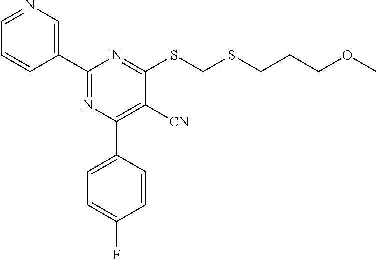 [SMILES: COCCCSCSc1nc(-c2cccnc2)nc(-c2ccc(F)cc2)c1C#N]

The target compound was prepared in 20% isolated yield, using synthetic procedures described for the preparation of Example 74. ESI-MS (m/z): 427.1 [M+H]⁺.

### Example 87-2: 4-(4-fluorophenyl)-6-((((3-methoxypropyl)sulfinyl)methyl)thio)-2-(pyridin-3-yl)pyrimidine-5-carbonitrile

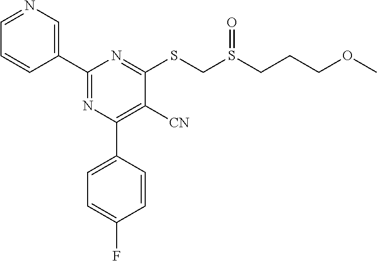 [SMILES: COCCCS(=O)CSc1nc(-c2cccnc2)nc(-c2ccc(F)cc2)c1C#N]

The target compound was prepared in 99% isolated yield, using synthetic procedures described for the preparation of Example 74-4. ¹H NMR (400 MHz, Chloroform-d) δ 9.73 (s, 1H), 8.80 (d, J=8.2 Hz, 2H), 8.38-7.92 (m, 2H), 7.50 (dd, J=7.9, 4.9 Hz, 1H), 7.44-6.97 (m, 2H), 4.71 (d, J=13.3 Hz, 1H), 4.64 (d, J=13.3 Hz, 1H), 3.62-3.40 (m, 2H), 3.32 (s, 3H), 3.16-3.01 (m, 1H), 3.01-2.84 (m, 1H), 2.27-1.93 (m, 2H). ESI-MS (m/z): 443.1 [M+H]⁺.

### Example 87: 4-(4-fluorophenyl)-6-((3-methoxypropyl)sulfinyl)-2-(pyridin-3-yl)thieno[2,3-d]pyrimidin-5-amine

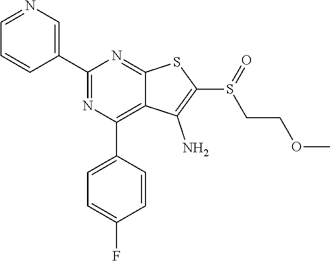 [SMILES: COCCS(=O)c1sc2nc(-c3cccnc3)nc(-c3ccc(F)cc3)c2c1N]

The target compound was prepared in 42% yield, using synthetic procedures described for the preparation of Example 74. ¹H NMR (400 MHz, Methylene Chloride-d₂) δ 9.72 (s, 1H), 8.91-8.78 (m, 1H), 8.71 (dt, J=4.7, 1.2 Hz, 1H), 7.88-7.67 (m, 2H), 7.55-7.39 (m, 1H), 7.39-7.24 (m, 2H), 4.84 (s, 2H), 3.51 (t, J=5.9 Hz, 2H), 3.40-3.28 (m, 1H), 3.33 (s, 3H) 3.29-3.15 (m, 1H), 2.14-1.89 (m, 2H). ESI-MS (m/z): 443.1 [M+H]⁺. Enantiomers were separated on a 1 cm Chiralpak AD-H column using 100% MeOH with 5 m/min flow rate, 500 μL injection (concentration 20 mg/ml) the 1V peak (Example 87B) was at 12.1 min and the 2^{nd} peak (Example 87A) was at 89.5 min.

### Example 88-1: 4-(4-fluorophenyl)-6-((((2-methoxyethyl)thio)methyl)thio)-2-(pyridin-3-yl)pyrimidine-5-carbonitrile

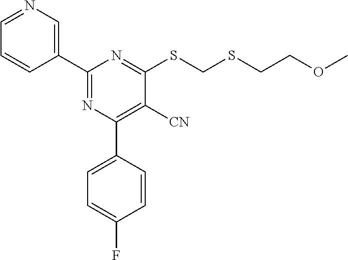 [SMILES: COCCSCSc1nc(-c2cccnc2)nc(-c2ccc(F)cc2)c1C#N]

The target compound was prepared in 9% isolated yield, using synthetic procedures described for the preparation of Example 74-3. ESI-MS (m/z): 413.1 [M+H]⁺.

### Example 88-2: 4-(4-fluorophenyl)-6-((((2-methoxyethyl)sulfinyl)methyl)thio)-2-(pyridin-3-yl)pyrimidine-5-carbonitrile

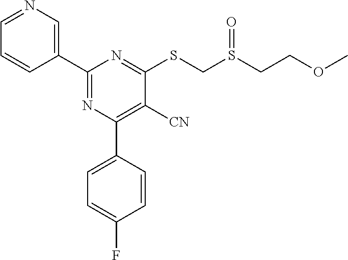 [SMILES: COCCS(=O)CSc1nc(-c2cccnc2)nc(-c2ccc(F)cc2)c1C#N]

The target compound was prepared in 99% isolated yield, using synthetic procedures described for the preparation of Example 74-4. ESI-MS (m/z): 429.1 [M+H]⁺.

### Example 88: 4-(4-fluorophenyl)-6-((2-methoxyethyl)sulfinyl)-2-(pyridin-3-yl)thieno[2,3-d]pyrimidin-5-amine

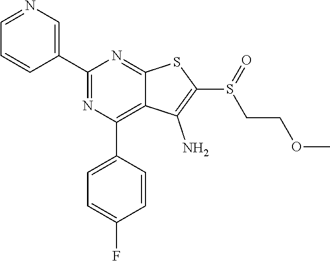 [SMILES: COCCS(=O)c1sc2nc(-c3cccnc3)nc(-c3ccc(F)cc3)c2c1N]

The target compound was prepared in 40% isolated yield, using synthetic procedures described for the preparation of Example 74. ¹H NMR (400 MHz, Methylene Chloride-d₂) δ 9.72 (s, 1H), 8.82 (dt, J=8.0, 2.0 Hz, 1H), 8.72 (d, J=4.7 Hz, 1H), 7.86-7.72 (m, 2H), 7.56-7.40 (m, 1H), 7.40-7.25 (m, 2H), 4.79 (s, 2H), 3.85 (ddd, J=10.1, 7.6, 3.9 Hz, 1H), 3.68 (ddd, J=10.5, 6.2, 4.3 Hz, 1H), 3.59 (ddd, J=12.8, 6.2, 3.9 Hz, 1H), 3.38 (s, 3H), 3.36-3.23 (m, 1H). ESI-MS (m/z): 429.1 [M+H]⁺. Enantiomers were separated on a 1 cm Chiralpak AD-H column using 100% MeOH with 5 m/min flow rate, 400 μL injection (concentration 20 mg/ml) the 1st peak (Example 88B) was at 15.80 min and the 2nd peak (Example 88A) was at 29.3 min.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

The following is claimed:

1. A compound having formula (I): 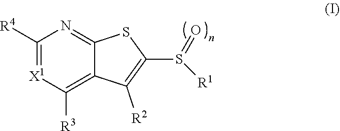
wherein n=0-2;
X¹ is N or CR^{a};
R¹ is selected from the group consisting of branched or linear alkyl including —(CH₂)n₁CH₃ (n₁=0-7), 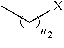
wherein n₂=0-6 and X is any of the following:
CF_{y}H_{z} (y+z=3), CCl_{y}H_{z} (y+z=3), OH, OAc, OMe, R⁷¹, OR⁷², CN, N(R⁷⁶)₂, 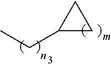
(n₃=0-5, m=1-5), and 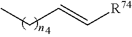
(n₄=0-5);
R² is selected from the group consisting of H, linear or branched alkyl, OH, Cl, F, NH₂, N(R⁷⁶)₂, and OR⁷⁷;
R³ and R⁴ are each independently selected from the group consisting of: 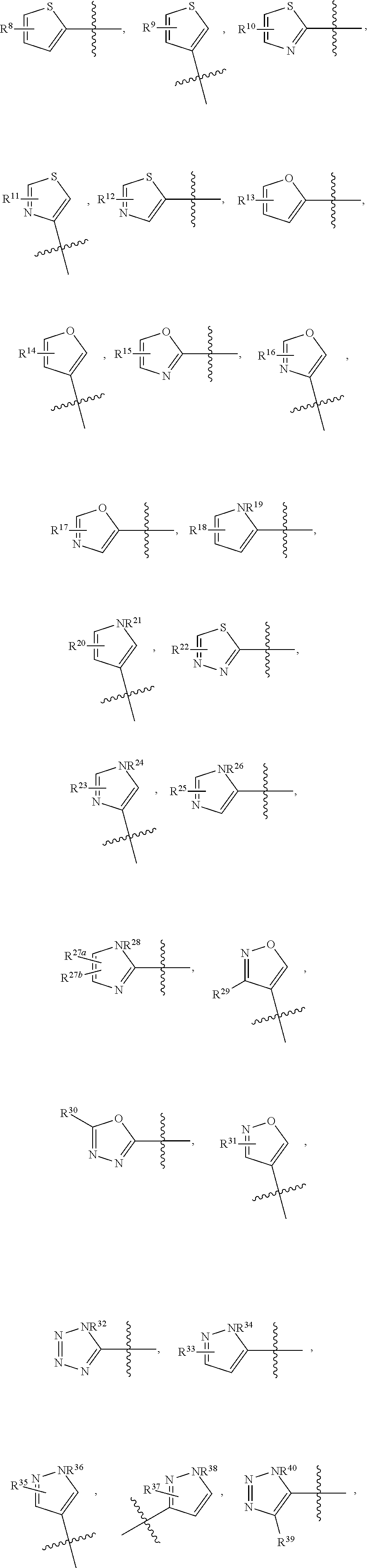 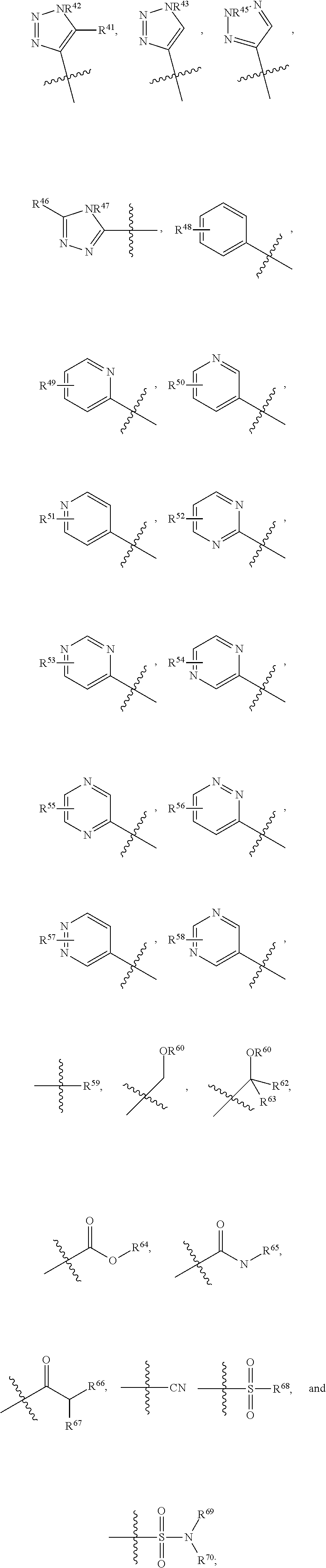
and
each R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R^{27a}, R^{27b}, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁶, R⁷⁷, and R^{a} are the same or different and are independently selected from the group consisting of hydrogen, substituted or unsubstituted C₁-C₂₄ alkyl, C₂-C₂₄ alkenyl, C₂-C₂₄ alkynyl, C₆-C₂₀ aryl, heterocycloalkenyl containing from 4-6 ring atoms, (wherein from 1-3 of the ring atoms is independently selected from N, NH, N(C₁-C₆ alkyl), NC(O) (C₁-C₆ alkyl), O, and S), heteroaryl or heterocyclyl containing from 4-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano(—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R═hydrogen, alkyl, aryl, alkaryl, aralkyl, etc.), arylimino (—CR═N(aryl), where R═hydrogen, alkyl, aryl, alkaryl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, arlyl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R═H, methyl or other alkyl], groups incorporating amino acids or other moieties expected to bear positive or negative charge at physiological pH, and combinations thereof; wherein R² and R³ can be linked to form a cyclic or heterocyclic ring, or a pharmaceutically acceptable salt thereof;
not having a formula selected from the group consisting of:

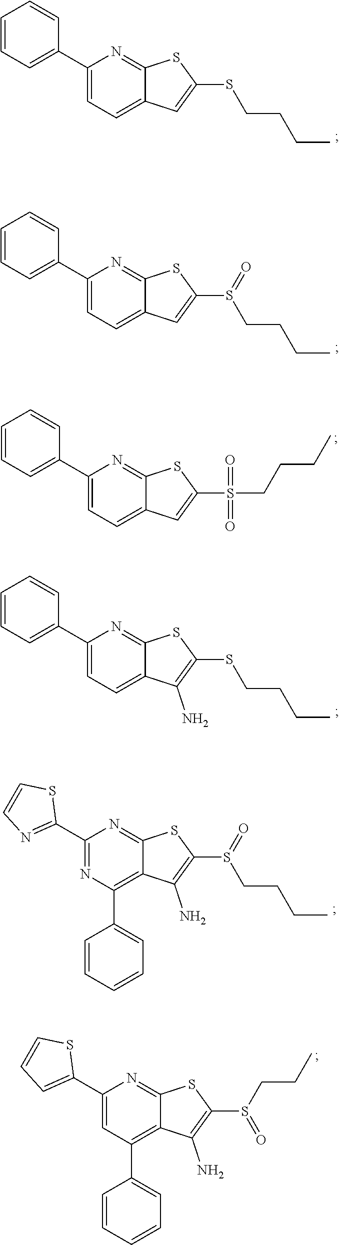 [SMILES: CCCCSc1cc2ccc(-c3ccccc3)nc2s1 ; CCCCS(=O)c1cc2ccc(-c3ccccc3)nc2s1 ; CCCCS(=O)(=O)c1cc2ccc(-c3ccccc3)nc2s1 ; CCCCSc1sc2nc(-c3ccccc3)ccc2c1N ; CCCCS(=O)c1sc2nc(-c3nccs3)nc(-c3ccccc3)c2c1N ; CCCS(=O)c1sc2nc(-c3cccs3)cc(-c3ccccc3)c2c1N]

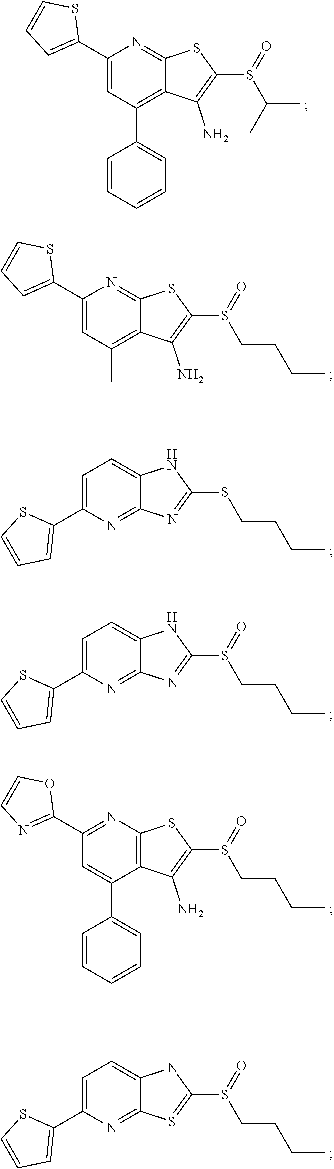

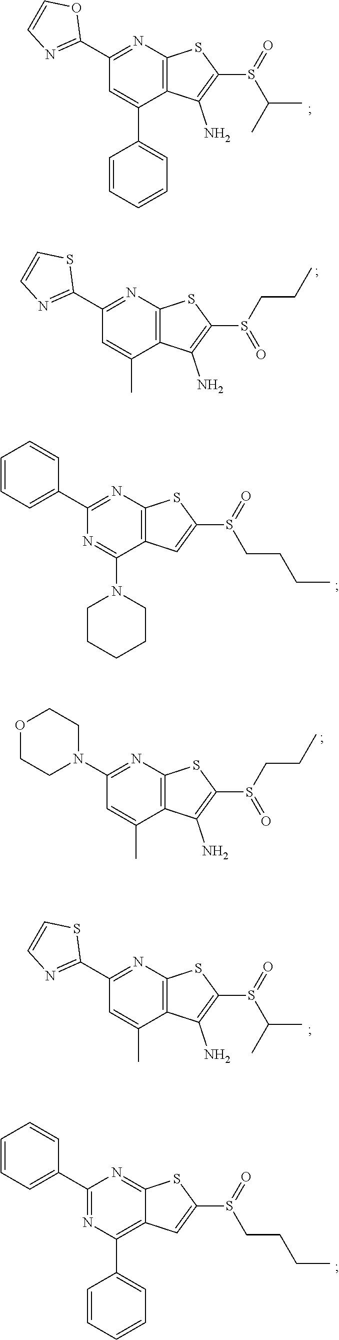 [SMILES: CC(C)S(=O)c1sc2nc(-c3ncco3)cc(-c3ccccc3)c2c1N ; CCCS(=O)c1sc2nc(-c3nccs3)cc(C)c2c1N ; CCCCS(=O)c1cc2c(N3CCCCC3)nc(-c3ccccc3)nc2s1 ; CCCS(=O)c1sc2nc(N3CCOCC3)cc(C)c2c1N ; Cc1cc(-c2nccs2)nc2sc(S(=O)C(C)C)c(N)c12 ; CCCCS(=O)c1cc2c(-c3ccccc3)nc(-c3ccccc3)nc2s1]

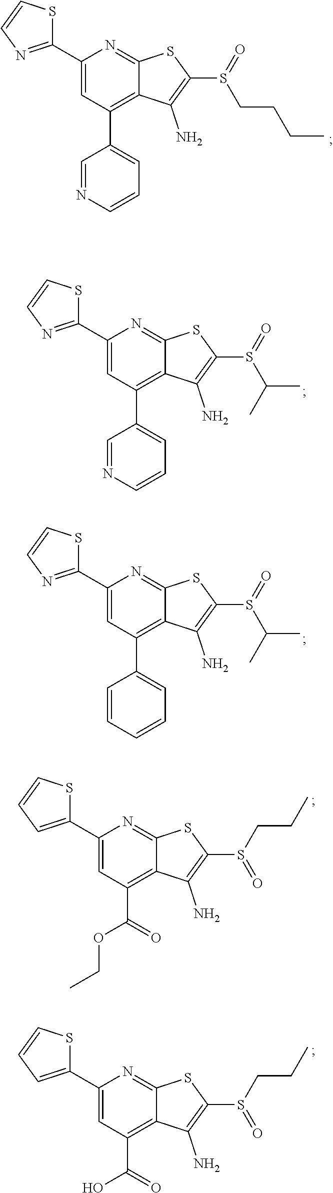 [SMILES: CCCCS(=O)c1sc2nc(-c3nccs3)cc(-c3cccnc3)c2c1N ; CC(C)S(=O)c1sc2nc(-c3nccs3)cc(-c3cccnc3)c2c1N ; CC(C)S(=O)c1sc2nc(-c3nccs3)cc(-c3ccccc3)c2c1N ; CCCS(=O)c1sc2nc(-c3cccs3)cc(C(=O)OCC)c2c1N ; CCCS(=O)c1sc2nc(-c3cccs3)cc(C(=O)O)c2c1N]

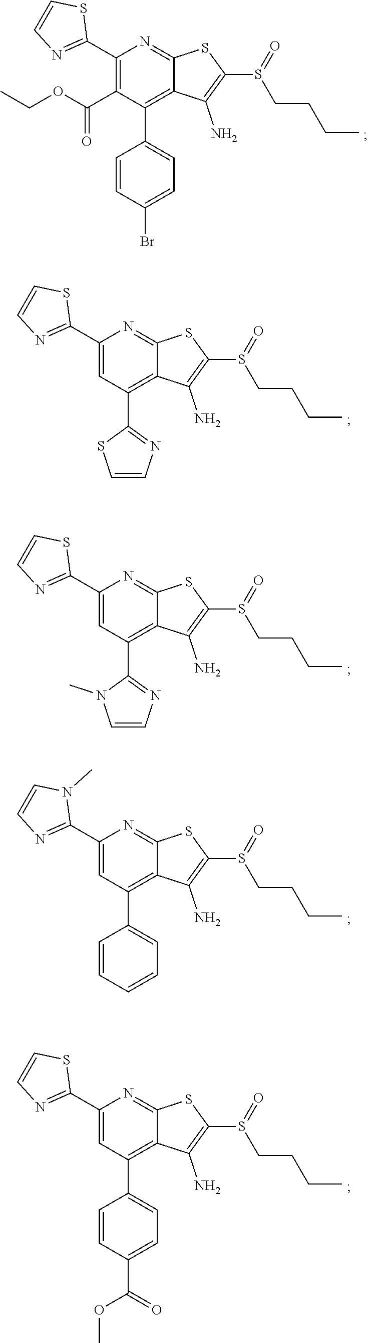 [SMILES: CCCCS(=O)c1sc2nc(-c3nccs3)c(C(=O)OCC)c(-c3ccc(Br)cc3)c2c1N ; CCCCS(=O)c1sc2nc(-c3nccs3)cc(-c3nccs3)c2c1N ; CCCCS(=O)c1sc2nc(-c3nccs3)cc(-c3nccn3C)c2c1N ; CCCCS(=O)c1sc2nc(-c3nccn3C)cc(-c3ccccc3)c2c1N ; CCCCS(=O)c1sc2nc(-c3nccs3)cc(-c3ccc(C(=O)OC)cc3)c2c1N]

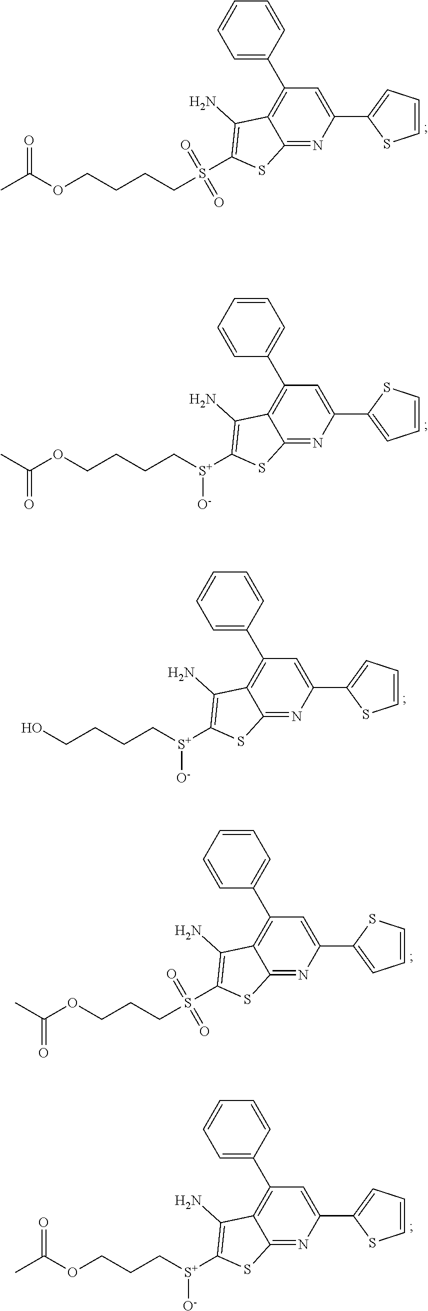 [SMILES: CC(=O)OCCCCS(=O)(=O)c1sc2nc(-c3cccs3)cc(-c3ccccc3)c2c1N ; CC(=O)OCCCC[S+]([O-])c1sc2nc(-c3cccs3)cc(-c3ccccc3)c2c1N ; Nc1c([S+]([O-])CCCCO)sc2nc(-c3cccs3)cc(-c3ccccc3)c12 ; CC(=O)OCCCS(=O)(=O)c1sc2nc(-c3cccs3)cc(-c3ccccc3)c2c1N ; CC(=O)OCCC[S+]([O-])c1sc2nc(-c3cccs3)cc(-c3ccccc3)c2c1N]

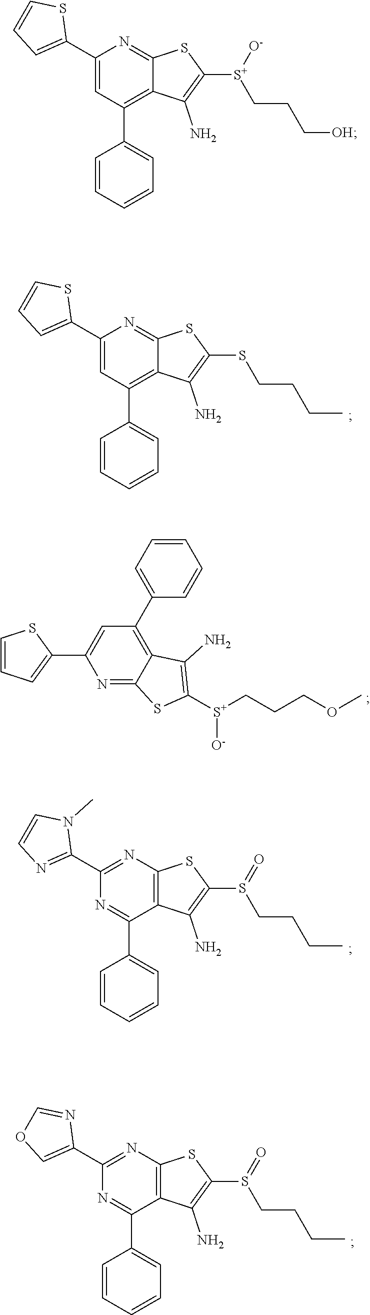 [SMILES: Nc1c([S+]([O-])CCCO)sc2nc(-c3cccs3)cc(-c3ccccc3)c12 ; CCCCSc1sc2nc(-c3cccs3)cc(-c3ccccc3)c2c1N ; COCCC[S+]([O-])c1sc2nc(-c3cccs3)cc(-c3ccccc3)c2c1N ; CCCCS(=O)c1sc2nc(-c3nccn3C)nc(-c3ccccc3)c2c1N ; CCCCS(=O)c1sc2nc(-c3cocn3)nc(-c3ccccc3)c2c1N]

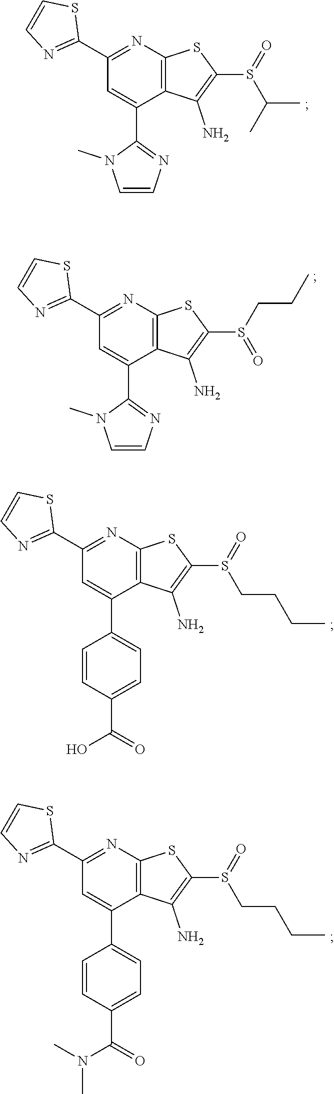 [SMILES: CC(C)S(=O)c1sc2nc(-c3nccs3)cc(-c3nccn3C)c2c1N ; CCCS(=O)c1sc2nc(-c3nccs3)cc(-c3nccn3C)c2c1N ; CCCCS(=O)c1sc2nc(-c3nccs3)cc(-c3ccc(C(=O)O)cc3)c2c1N ; CCCCS(=O)c1sc2nc(-c3nccs3)cc(-c3ccc(C(=O)N(C)C)cc3)c2c1N]

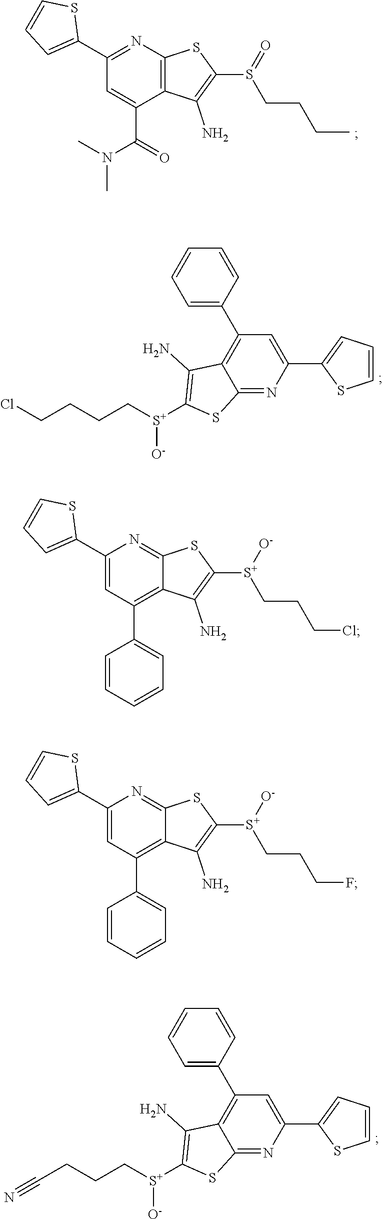 [SMILES: CCCCS(=O)c1sc2nc(-c3cccs3)cc(C(=O)N(C)C)c2c1N ; Nc1c([S+]([O-])CCCCCl)sc2nc(-c3cccs3)cc(-c3ccccc3)c12 ; Nc1c([S+]([O-])CCCCl)sc2nc(-c3cccs3)cc(-c3ccccc3)c12 ; Nc1c([S+]([O-])CCCF)sc2nc(-c3cccs3)cc(-c3ccccc3)c12 ; N#CCCC[S+]([O-])c1sc2nc(-c3cccs3)cc(-c3ccccc3)c2c1N]

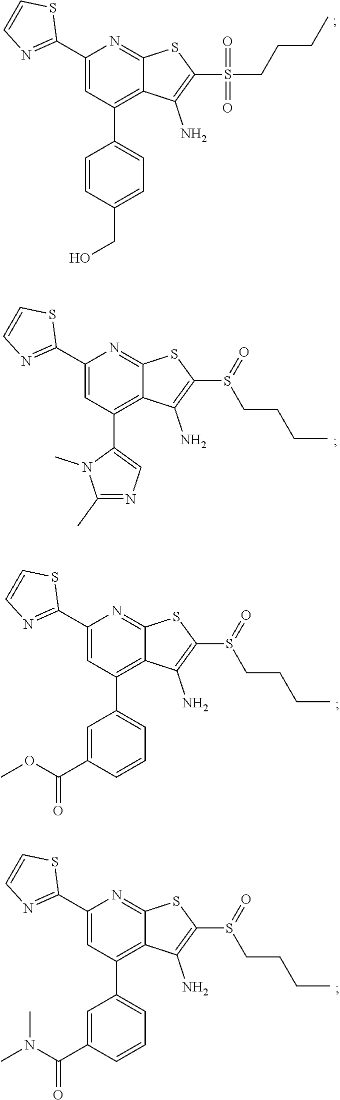

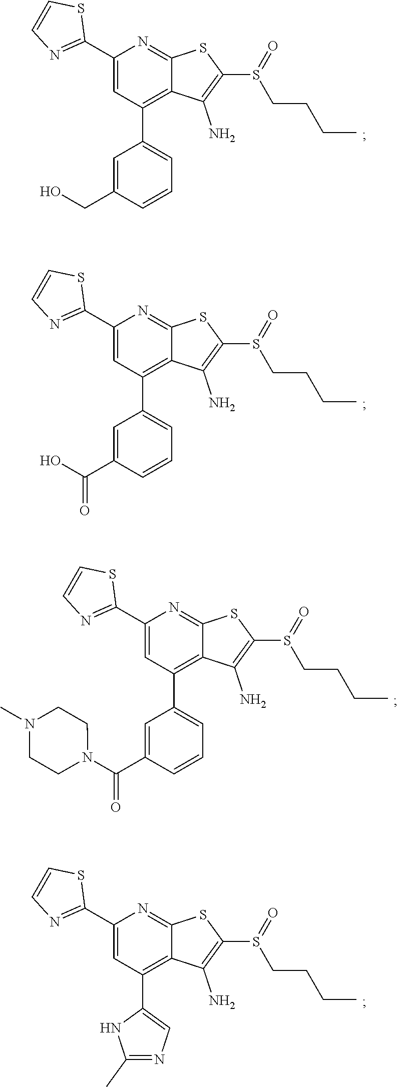

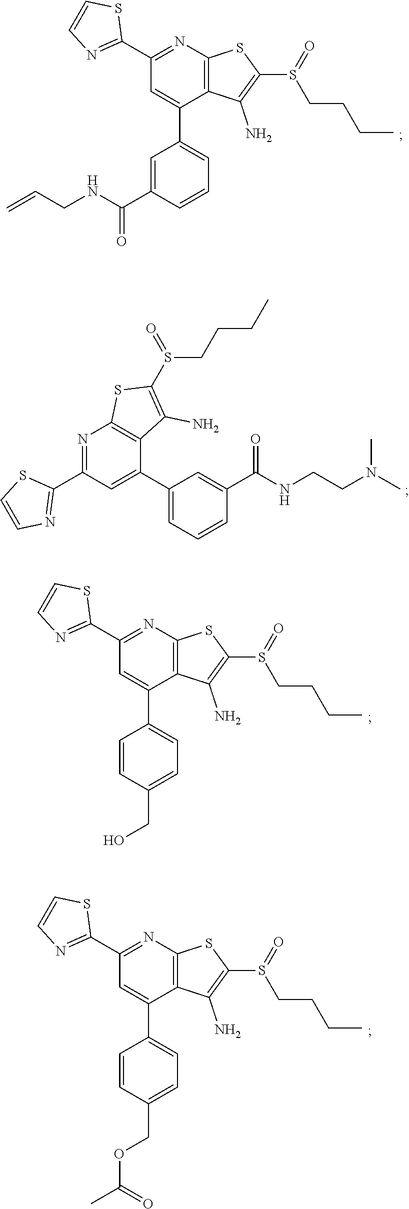 [SMILES: C=CCNC(=O)c1cccc(-c2cc(-c3nccs3)nc3sc(S(=O)CCCC)c(N)c23)c1 ; CCCCS(=O)c1sc2nc(-c3nccs3)cc(-c3cccc(C(=O)NCCN(C)C)c3)c2c1N ; CCCCS(=O)c1sc2nc(-c3nccs3)cc(-c3ccc(CO)cc3)c2c1N ; CCCCS(=O)c1sc2nc(-c3nccs3)cc(-c3ccc(COC(C)=O)cc3)c2c1N]

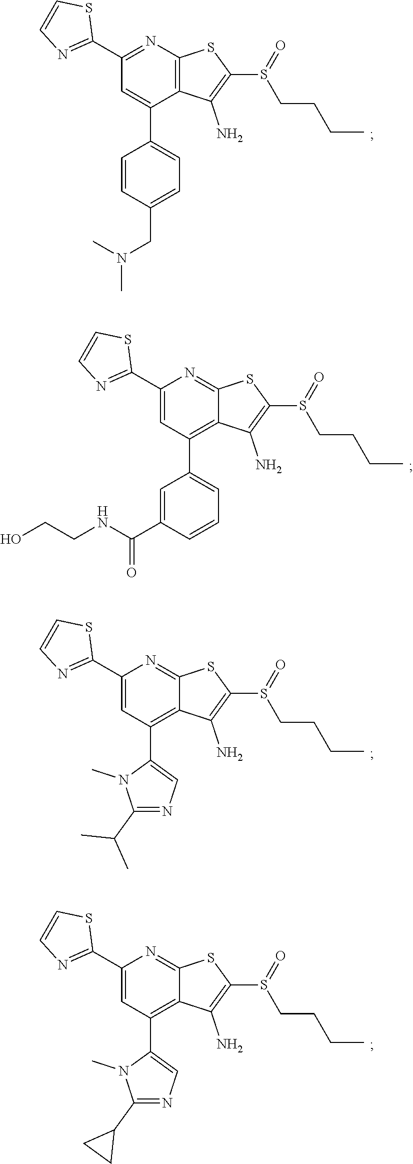 [SMILES: CCCCS(=O)c1sc2nc(-c3nccs3)cc(-c3ccc(CN(C)C)cc3)c2c1N ; CCCCS(=O)c1sc2nc(-c3nccs3)cc(-c3cccc(C(=O)NCCO)c3)c2c1N ; CCCCS(=O)c1sc2nc(-c3nccs3)cc(-c3cnc(C(C)C)n3C)c2c1N ; CCCCS(=O)c1sc2nc(-c3nccs3)cc(-c3cnc(C4CC4)n3C)c2c1N]

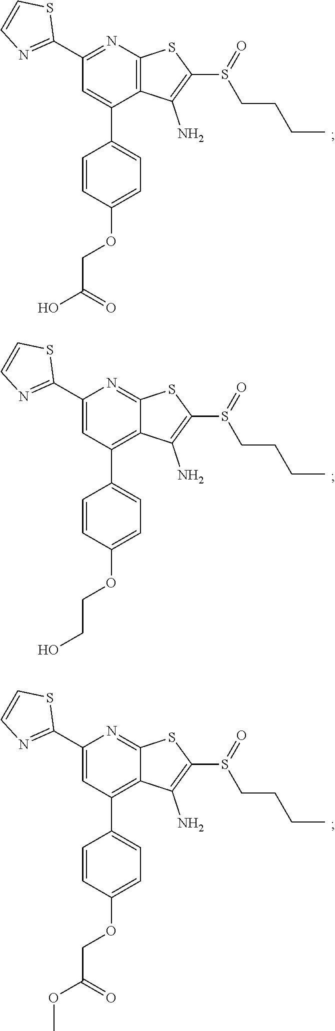

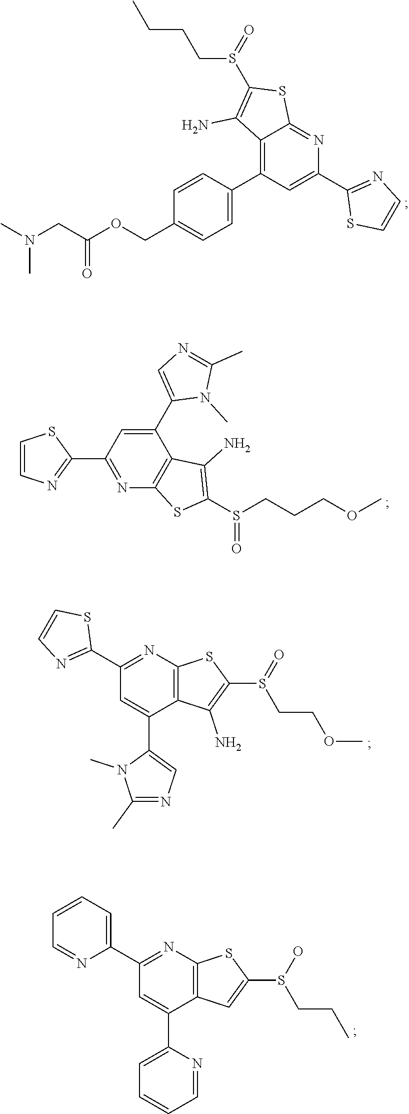

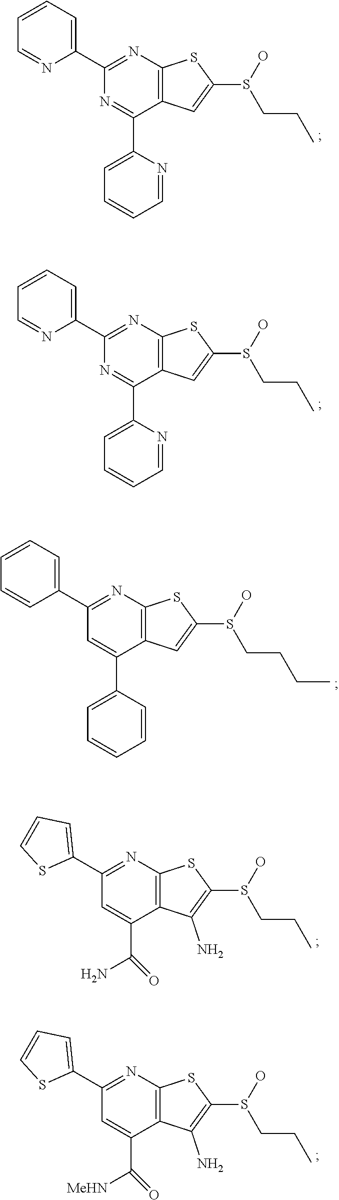

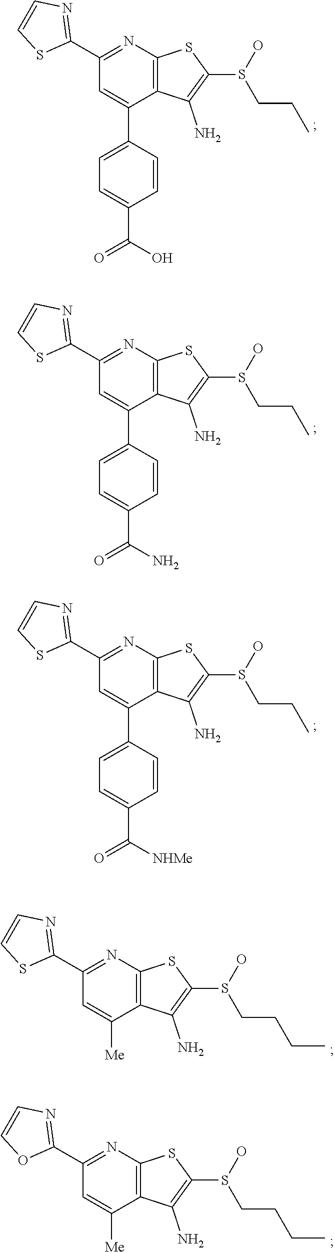

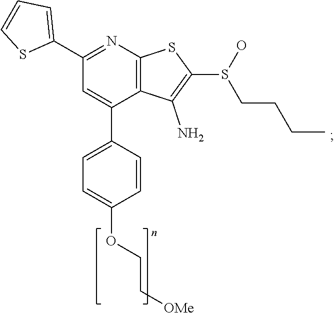

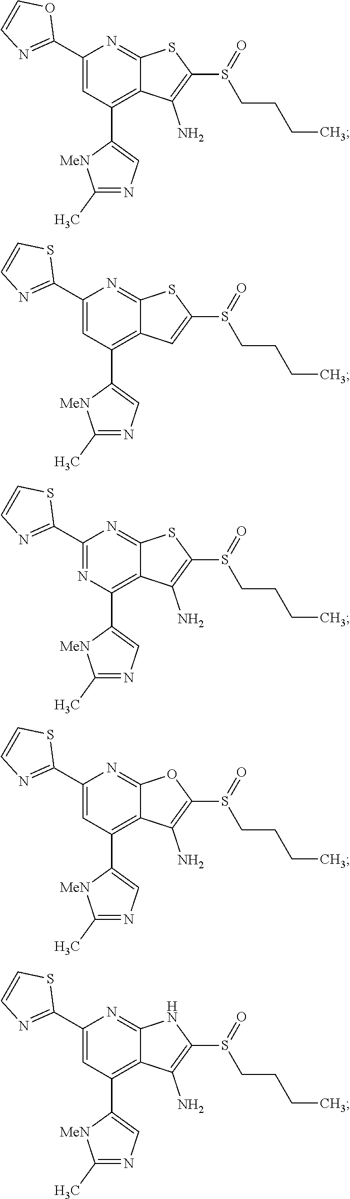

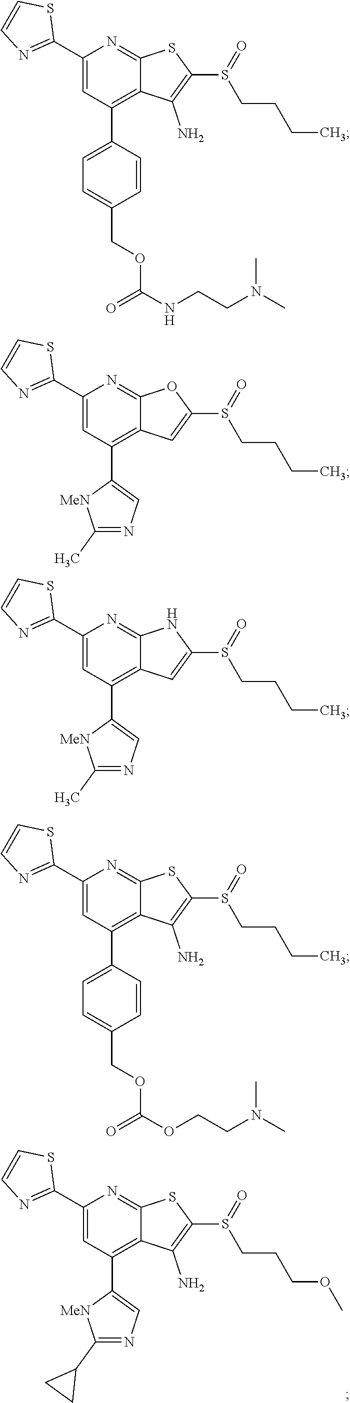

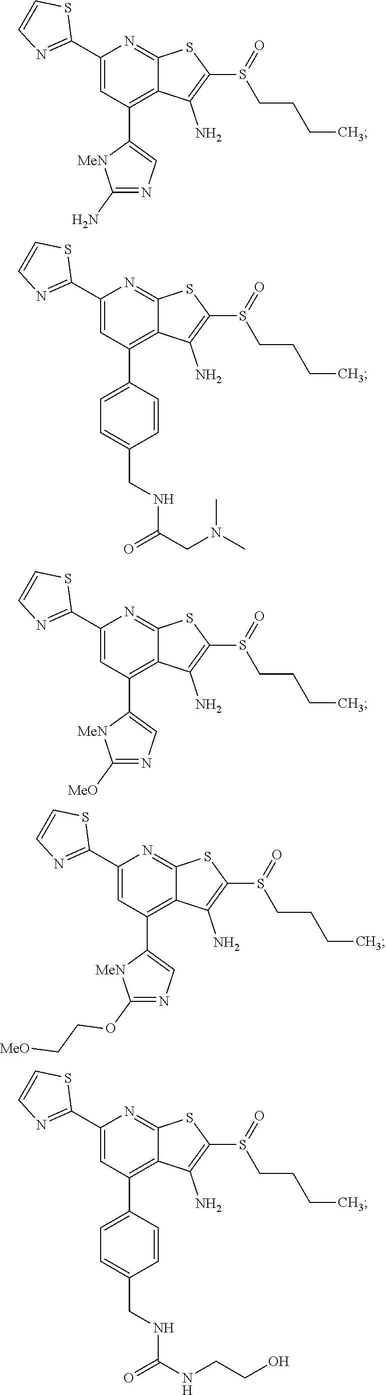

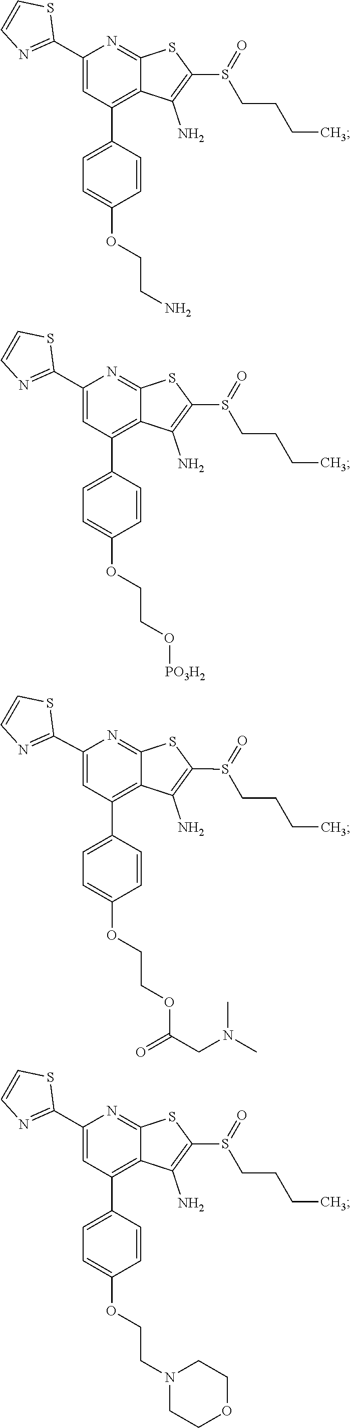

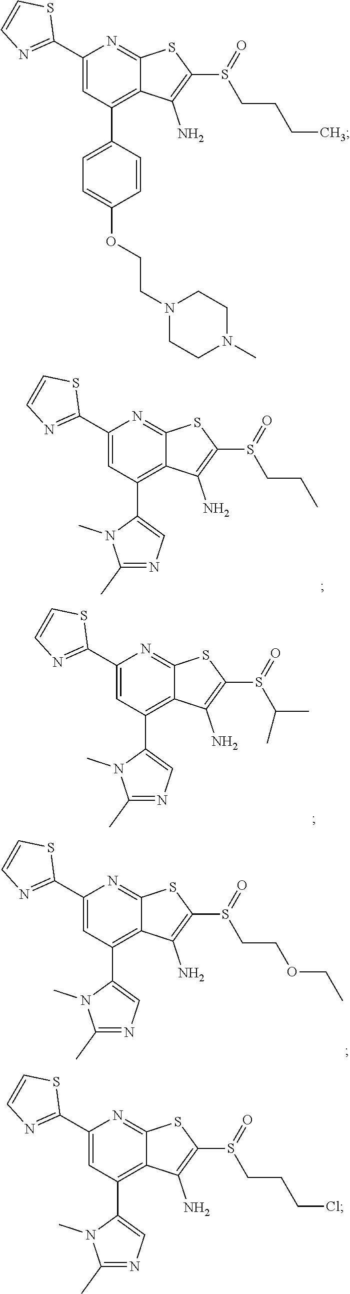

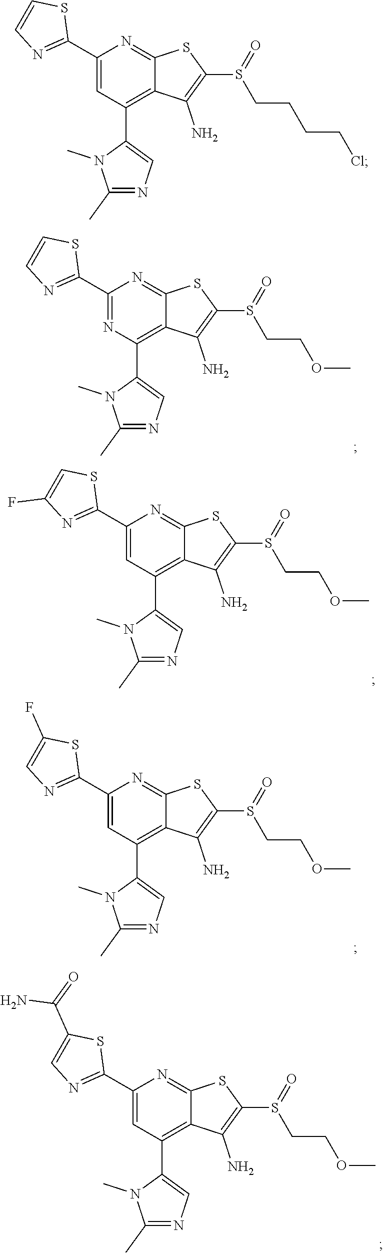

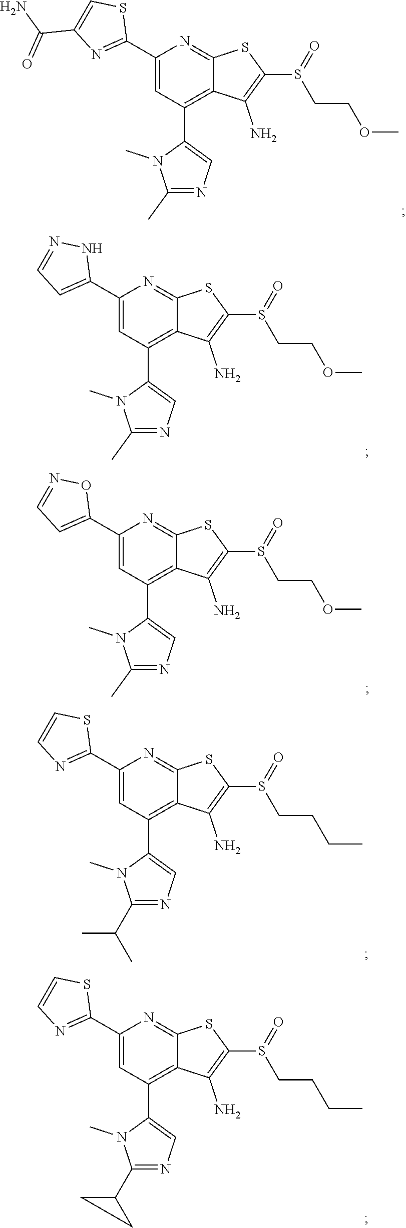

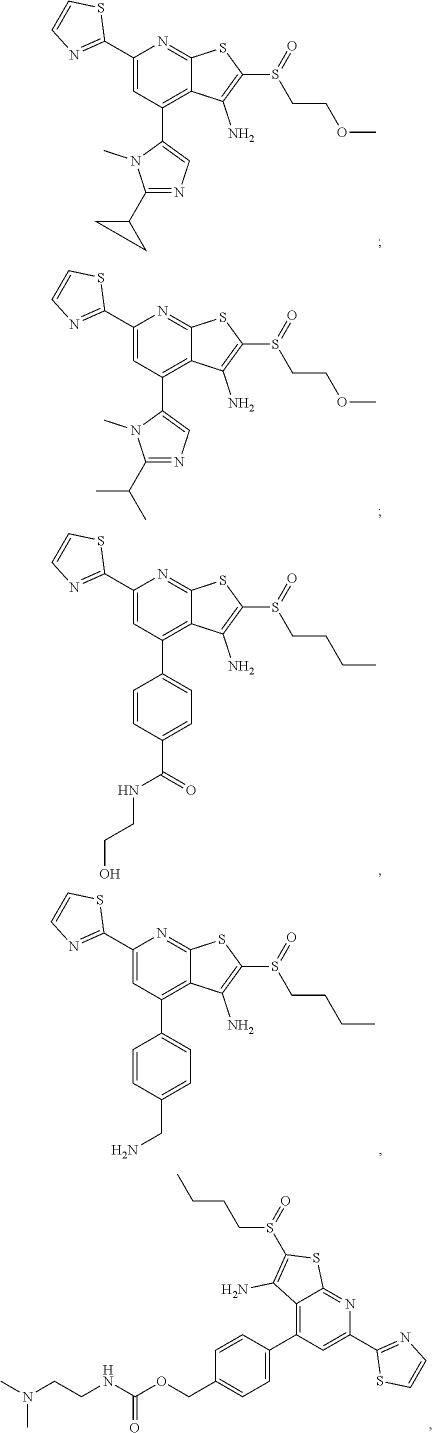

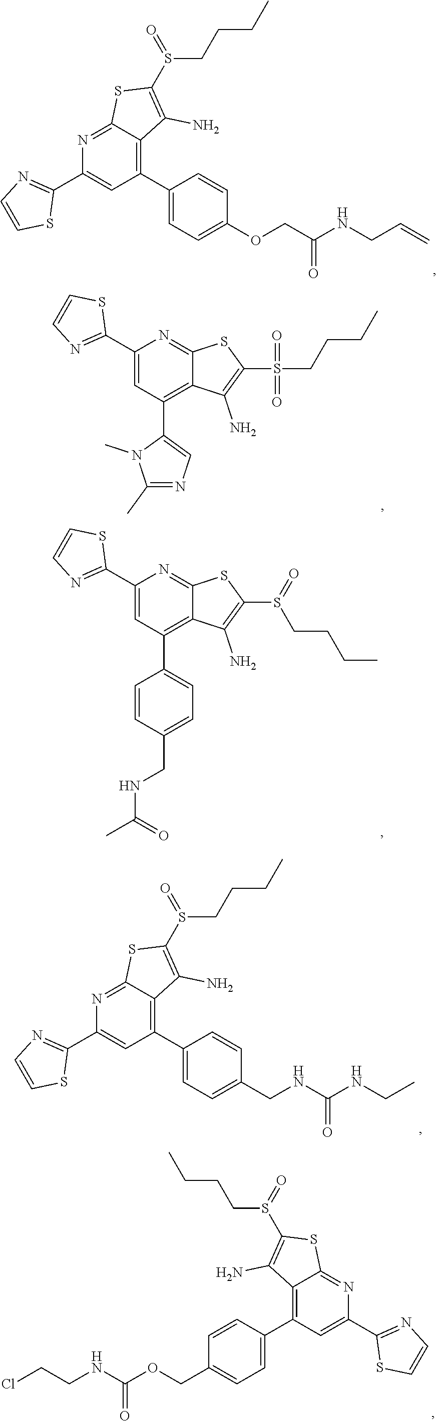

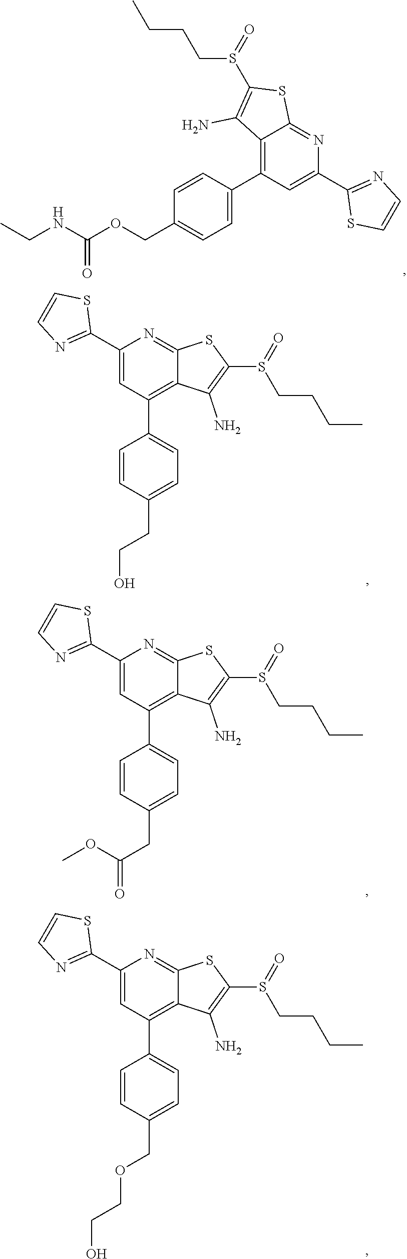

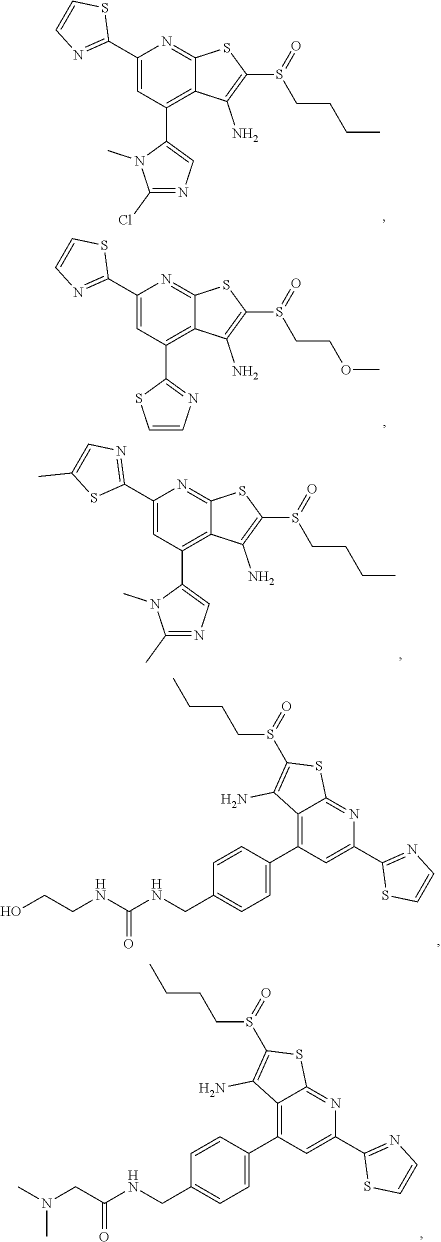

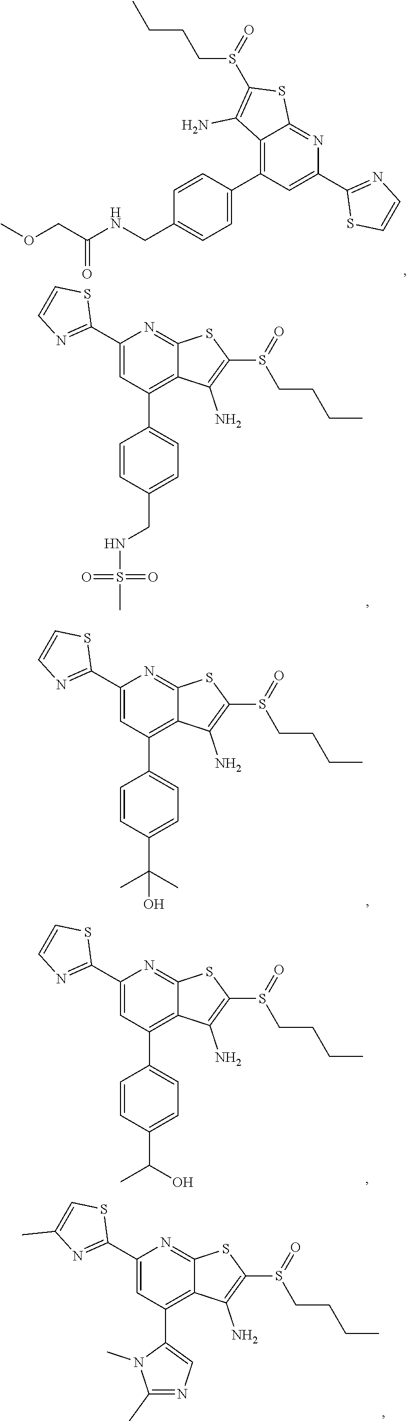

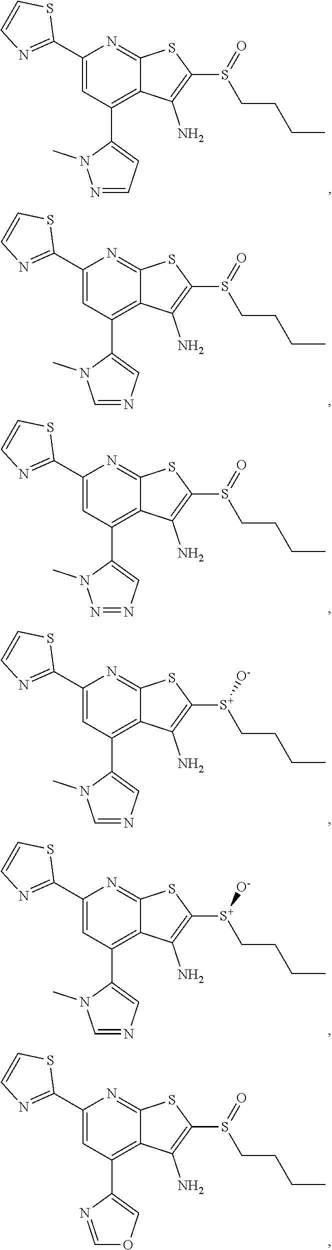 [SMILES: CCCCS(=O)c1sc2nc(-c3nccs3)cc(-c3ccnn3C)c2c1N ; CCCCS(=O)c1sc2nc(-c3nccs3)cc(-c3cncn3C)c2c1N ; CCCCS(=O)c1sc2nc(-c3nccs3)cc(-c3cnnn3C)c2c1N ; CCCC[S@@+]([O-])c1sc2nc(-c3nccs3)cc(-c3cncn3C)c2c1N ; CCCC[S@+]([O-])c1sc2nc(-c3nccs3)cc(-c3cncn3C)c2c1N ; CCCCS(=O)c1sc2nc(-c3nccs3)cc(-c3cocn3)c2c1N]

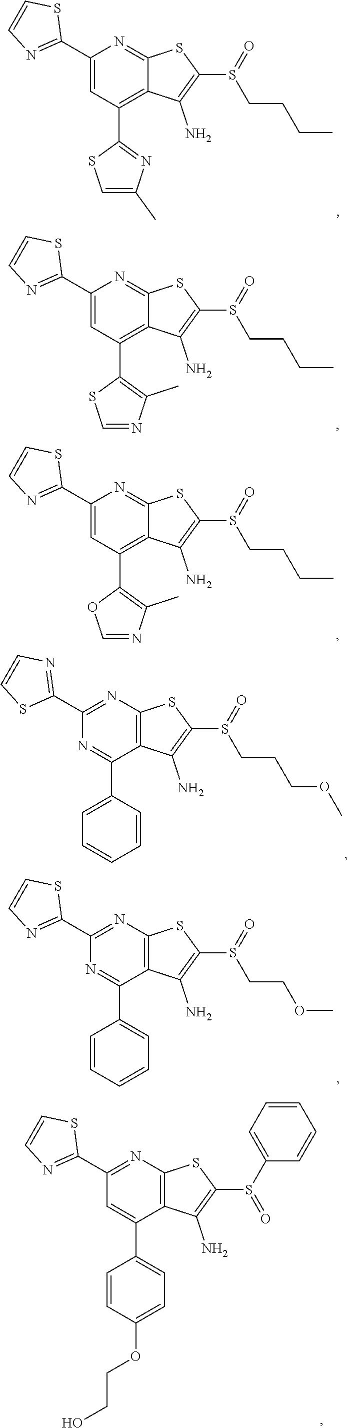 [SMILES: CCCCS(=O)c1sc2nc(-c3nccs3)cc(-c3nc(C)cs3)c2c1N ; CCCCS(=O)c1sc2nc(-c3nccs3)cc(-c3scnc3C)c2c1N ; CCCCS(=O)c1sc2nc(-c3nccs3)cc(-c3ocnc3C)c2c1N ; COCCCS(=O)c1sc2nc(-c3nccs3)nc(-c3ccccc3)c2c1N ; COCCS(=O)c1sc2nc(-c3nccs3)nc(-c3ccccc3)c2c1N ; Nc1c(S(=O)c2ccccc2)sc2nc(-c3nccs3)cc(-c3ccc(OCCO)cc3)c12]

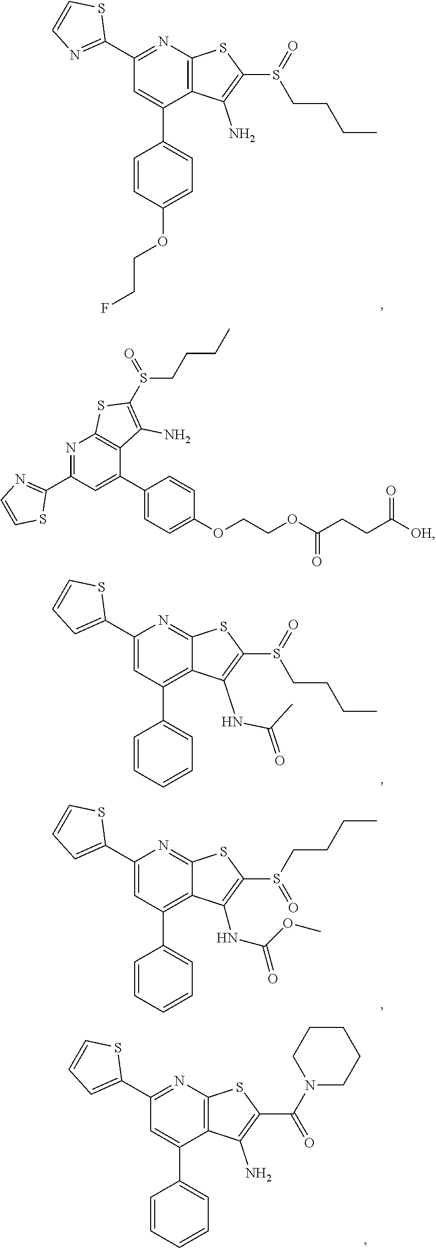 [SMILES: CCCCS(=O)c1sc2nc(-c3nccs3)cc(-c3ccc(OCCF)cc3)c2c1N ; CCCCS(=O)c1sc2nc(-c3nccs3)cc(-c3ccc(OCCOC(=O)CCC(=O)O)cc3)c2c1N ; CCCCS(=O)c1sc2nc(-c3cccs3)cc(-c3ccccc3)c2c1NC(C)=O ; CCCCS(=O)c1sc2nc(-c3cccs3)cc(-c3ccccc3)c2c1NC(=O)OC ; Nc1c(C(=O)N2CCCCC2)sc2nc(-c3cccs3)cc(-c3ccccc3)c12]

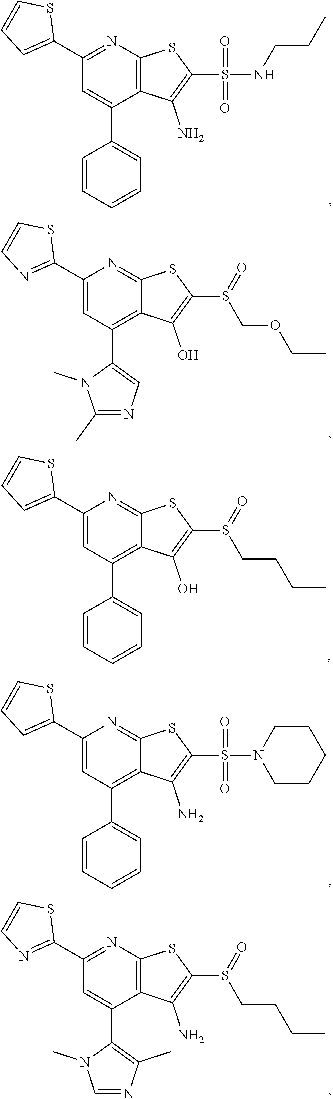

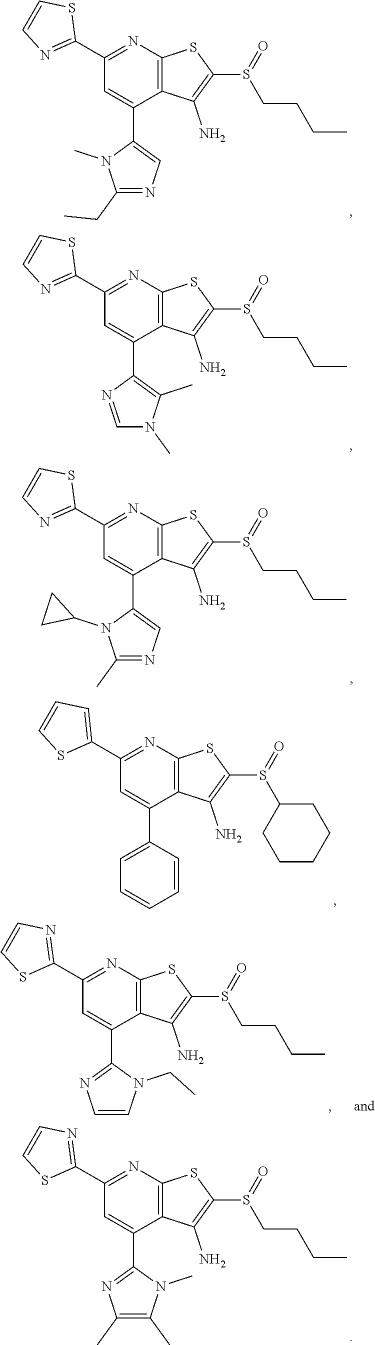

2. The compound of claim 1, having a formula selected from the group consisting of: 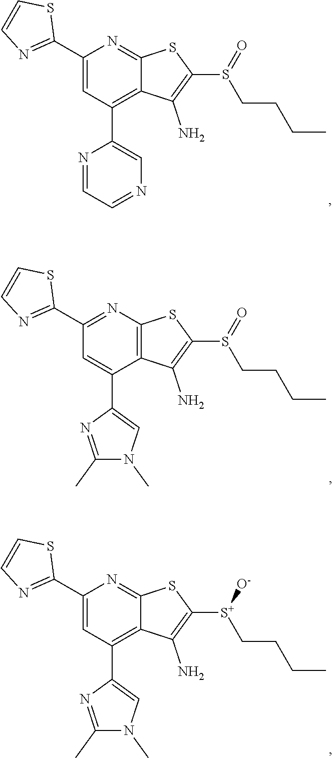 [SMILES: CCCCS(=O)c1sc2nc(-c3nccs3)cc(-c3cnccn3)c2c1N ; CCCCS(=O)c1sc2nc(-c3nccs3)cc(-c3cn(C)c(C)n3)c2c1N ; CCCC[S@+]([O-])c1sc2nc(-c3nccs3)cc(-c3cn(C)c(C)n3)c2c1N] 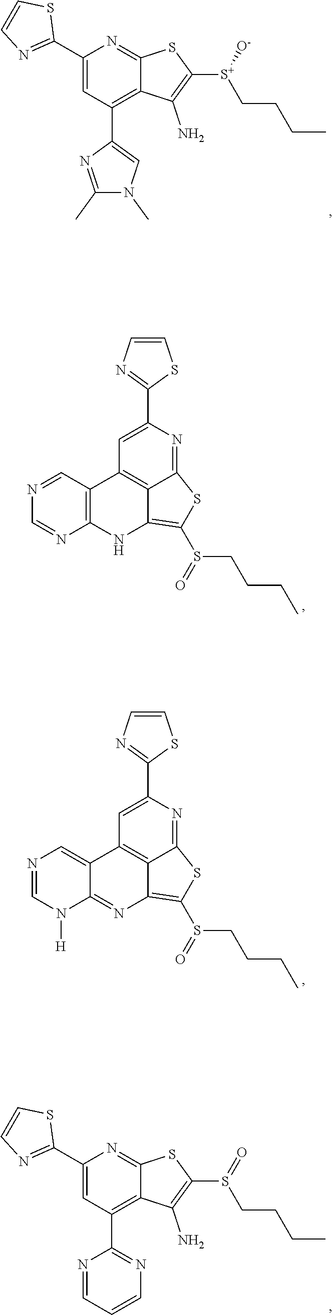 [SMILES: CCCC[S@@+]([O-])c1sc2nc(-c3nccs3)cc(-c3cn(C)c(C)n3)c2c1N ; CCCCS(=O)c1sc2nc(-c3nccs3)cc3c2c1Nc1ncncc1-3 ; CCCCS(=O)c1sc2nc(-c3nccs3)cc3c4c(nc1c23)NC=NC=4 ; CCCCS(=O)c1sc2nc(-c3nccs3)cc(-c3ncccn3)c2c1N] 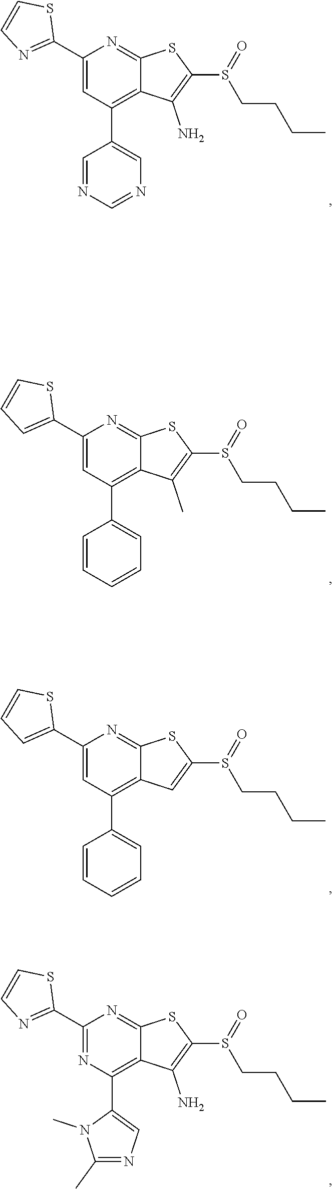 [SMILES: CCCCS(=O)c1sc2nc(-c3nccs3)cc(-c3cncnc3)c2c1N ; CCCCS(=O)c1sc2nc(-c3cccs3)cc(-c3ccccc3)c2c1C ; CCCCS(=O)c1cc2c(-c3ccccc3)cc(-c3cccs3)nc2s1 ; CCCCS(=O)c1sc2nc(-c3nccs3)nc(-c3cnc(C)n3C)c2c1N] 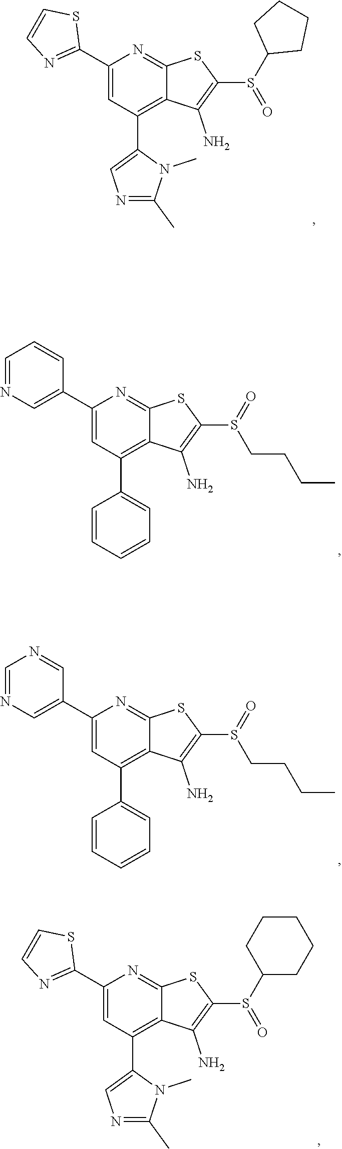 [SMILES: Cc1ncc(-c2cc(-c3nccs3)nc3sc(S(=O)C4CCCC4)c(N)c23)n1C ; CCCCS(=O)c1sc2nc(-c3cccnc3)cc(-c3ccccc3)c2c1N ; CCCCS(=O)c1sc2nc(-c3cncnc3)cc(-c3ccccc3)c2c1N ; Cc1ncc(-c2cc(-c3nccs3)nc3sc(S(=O)C4CCCCC4)c(N)c23)n1C] 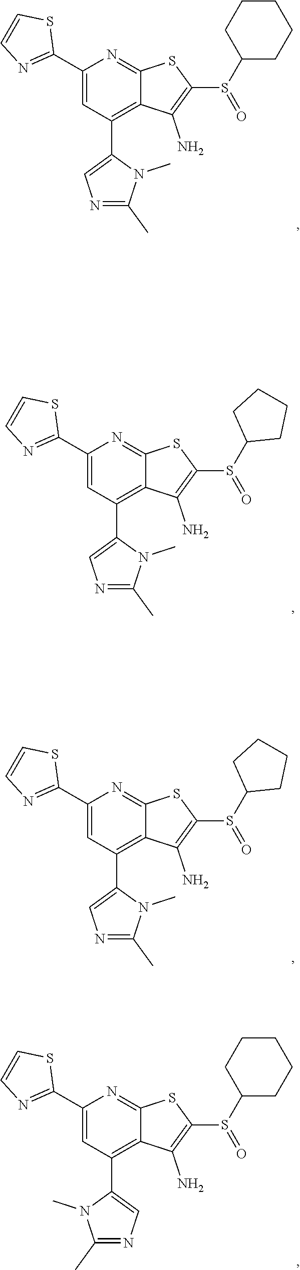 [SMILES: Cc1ncc(-c2cc(-c3nccs3)nc3sc(S(=O)C4CCCCC4)c(N)c23)n1C ; Cc1ncc(-c2cc(-c3nccs3)nc3sc(S(=O)C4CCCC4)c(N)c23)n1C ; Cc1ncc(-c2cc(-c3nccs3)nc3sc(S(=O)C4CCCC4)c(N)c23)n1C ; Cc1ncc(-c2cc(-c3nccs3)nc3sc(S(=O)C4CCCCC4)c(N)c23)n1C] 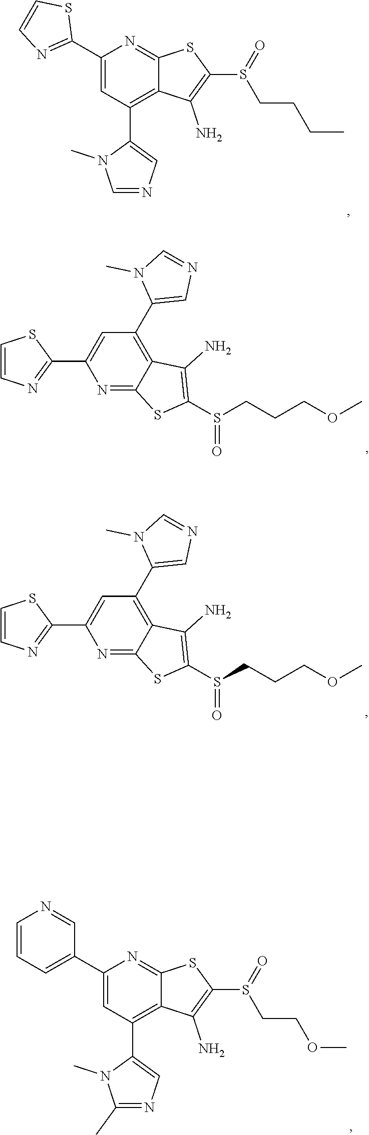 [SMILES: CCCCS(=O)c1sc2nc(-c3nccs3)cc(-c3cncn3C)c2c1N ; COCCCS(=O)c1sc2nc(-c3nccs3)cc(-c3cncn3C)c2c1N ; COCCC[S@@](=O)c1sc2nc(-c3nccs3)cc(-c3cncn3C)c2c1N ; COCCS(=O)c1sc2nc(-c3cccnc3)cc(-c3cnc(C)n3C)c2c1N] 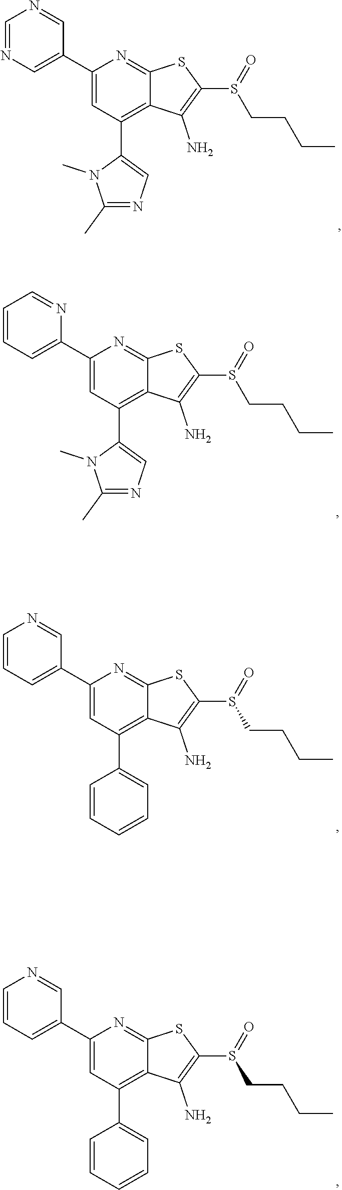 [SMILES: CCCCS(=O)c1sc2nc(-c3cncnc3)cc(-c3cnc(C)n3C)c2c1N ; CCCCS(=O)c1sc2nc(-c3ccccn3)cc(-c3cnc(C)n3C)c2c1N ; CCCC[S@@](=O)c1sc2nc(-c3cccnc3)cc(-c3ccccc3)c2c1N ; CCCC[S@](=O)c1sc2nc(-c3cccnc3)cc(-c3ccccc3)c2c1N] 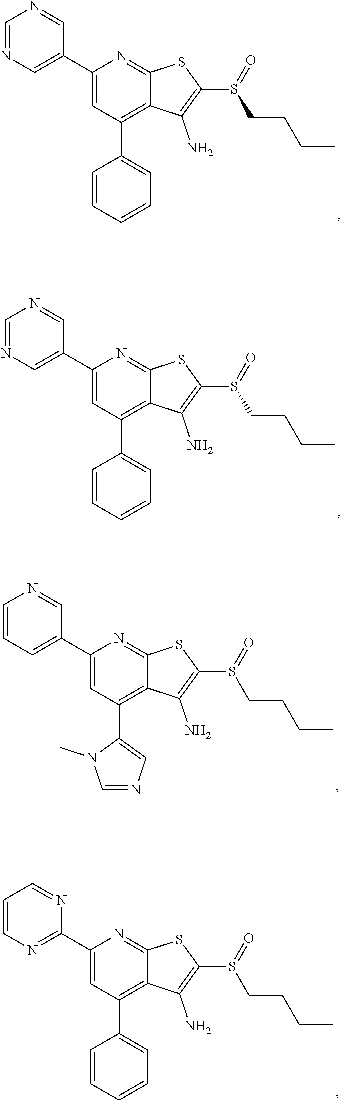 [SMILES: CCCC[S@](=O)c1sc2nc(-c3cncnc3)cc(-c3ccccc3)c2c1N ; CCCC[S@@](=O)c1sc2nc(-c3cncnc3)cc(-c3ccccc3)c2c1N ; CCCCS(=O)c1sc2nc(-c3cccnc3)cc(-c3cncn3C)c2c1N ; CCCCS(=O)c1sc2nc(-c3ncccn3)cc(-c3ccccc3)c2c1N] 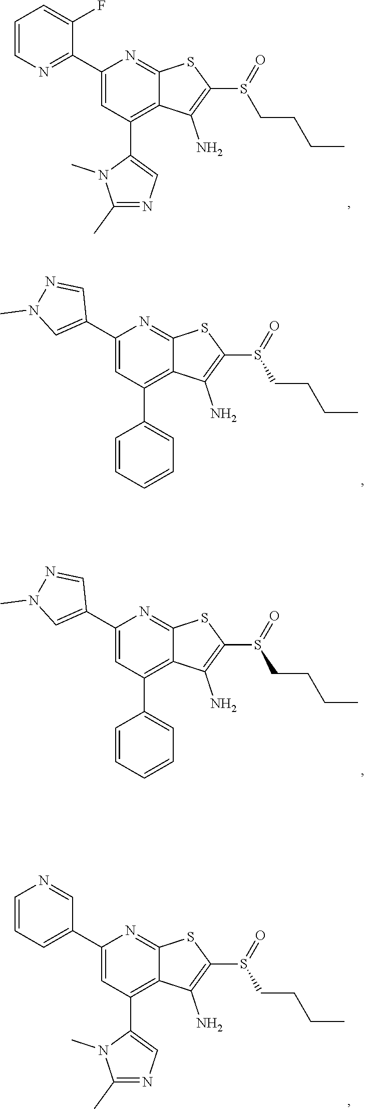 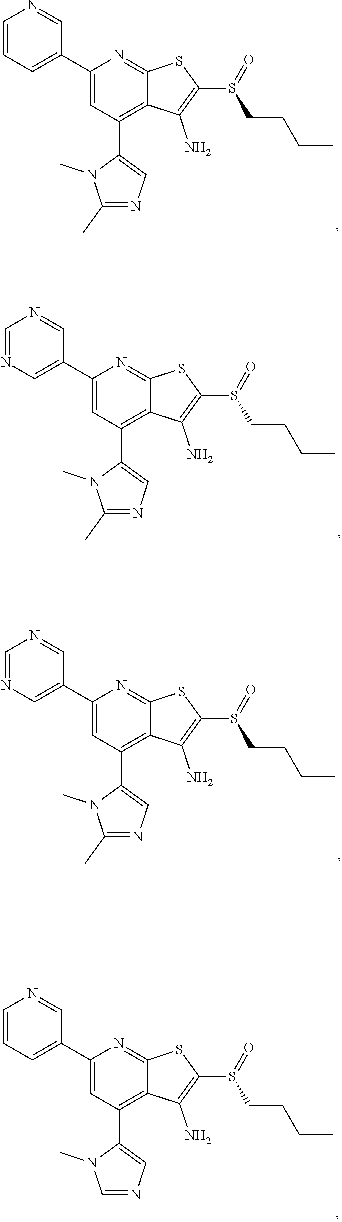 [SMILES: CCCC[S@](=O)c1sc2nc(-c3cccnc3)cc(-c3cnc(C)n3C)c2c1N ; CCCC[S@@](=O)c1sc2nc(-c3cncnc3)cc(-c3cnc(C)n3C)c2c1N ; CCCC[S@](=O)c1sc2nc(-c3cncnc3)cc(-c3cnc(C)n3C)c2c1N ; CCCC[S@@](=O)c1sc2nc(-c3cccnc3)cc(-c3cncn3C)c2c1N] 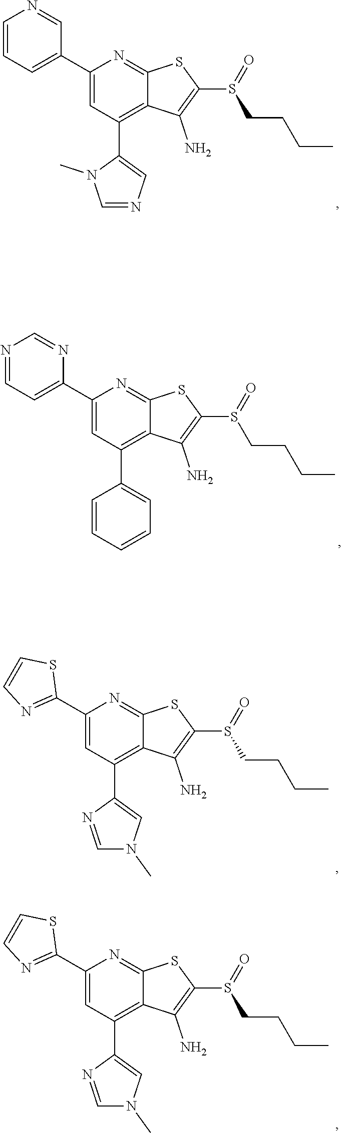 [SMILES: CCCC[S@](=O)c1sc2nc(-c3cccnc3)cc(-c3cncn3C)c2c1N ; CCCCS(=O)c1sc2nc(-c3ccncn3)cc(-c3ccccc3)c2c1N ; CCCC[S@@](=O)c1sc2nc(-c3nccs3)cc(-c3cn(C)cn3)c2c1N ; CCCC[S@](=O)c1sc2nc(-c3nccs3)cc(-c3cn(C)cn3)c2c1N] 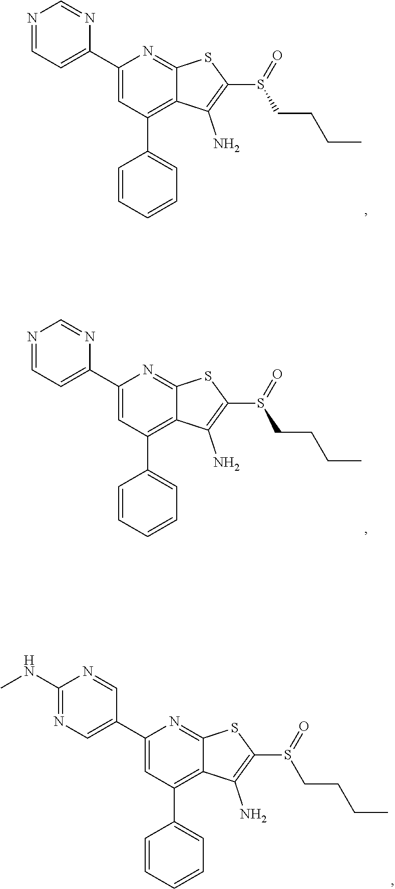 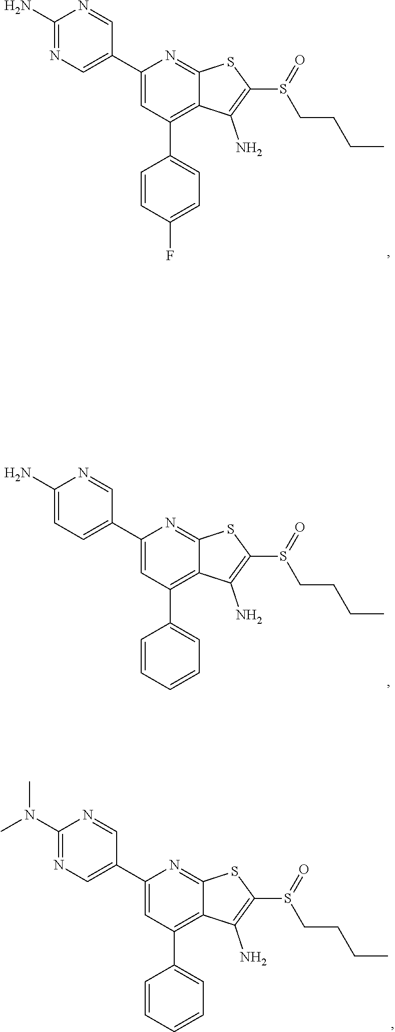 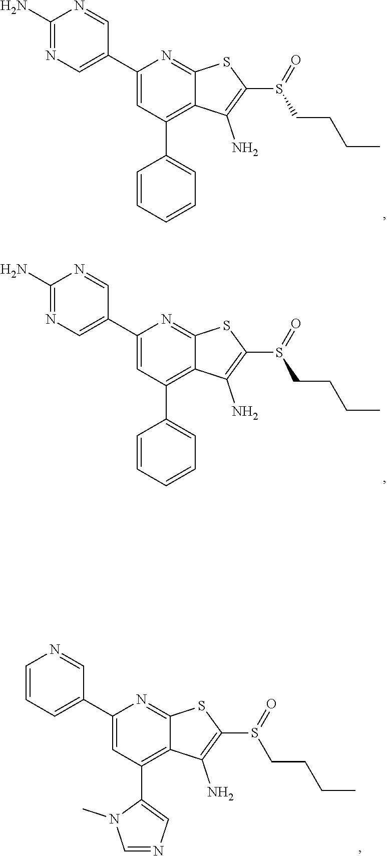 [SMILES: CCCC[S@@](=O)c1sc2nc(-c3cnc(N)nc3)cc(-c3ccccc3)c2c1N ; CCCC[S@](=O)c1sc2nc(-c3cnc(N)nc3)cc(-c3ccccc3)c2c1N ; CCCCS(=O)c1sc2nc(-c3cccnc3)cc(-c3cncn3C)c2c1N] 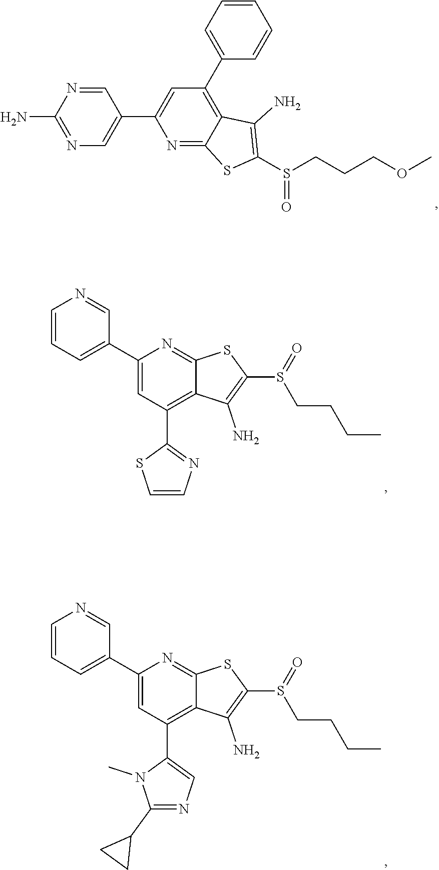 [SMILES: COCCCS(=O)c1sc2nc(-c3cnc(N)nc3)cc(-c3ccccc3)c2c1N ; CCCCS(=O)c1sc2nc(-c3cccnc3)cc(-c3nccs3)c2c1N ; CCCCS(=O)c1sc2nc(-c3cccnc3)cc(-c3cnc(C4CC4)n3C)c2c1N] 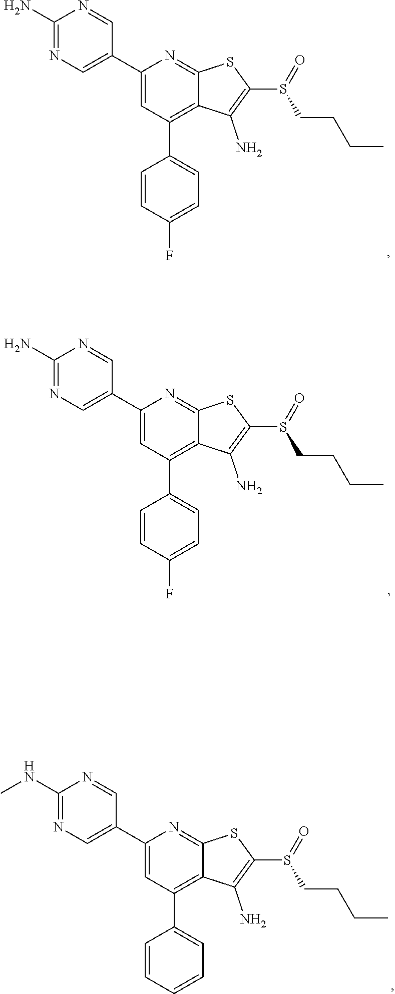 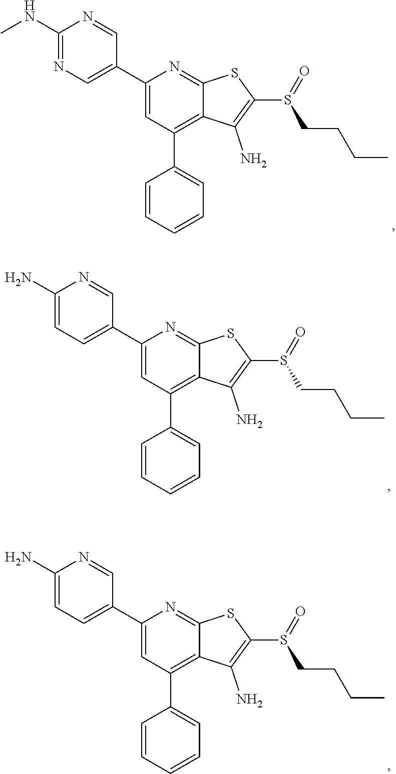 [SMILES: CCCC[S@](=O)c1sc2nc(-c3cnc(NC)nc3)cc(-c3ccccc3)c2c1N ; CCCC[S@@](=O)c1sc2nc(-c3ccc(N)nc3)cc(-c3ccccc3)c2c1N ; CCCC[S@](=O)c1sc2nc(-c3ccc(N)nc3)cc(-c3ccccc3)c2c1N] 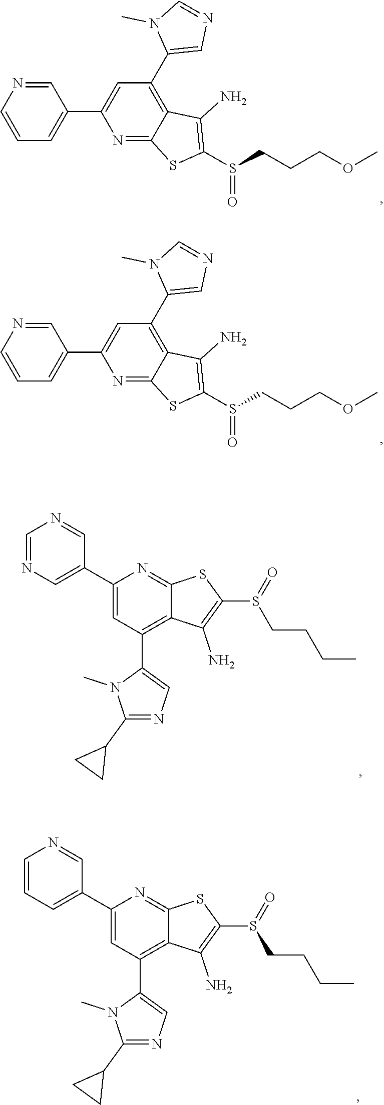 [SMILES: COCCC[S@@](=O)c1sc2nc(-c3cccnc3)cc(-c3cncn3C)c2c1N ; COCCC[S@](=O)c1sc2nc(-c3cccnc3)cc(-c3cncn3C)c2c1N ; CCCCS(=O)c1sc2nc(-c3cncnc3)cc(-c3cnc(C4CC4)n3C)c2c1N ; CCCC[S@](=O)c1sc2nc(-c3cccnc3)cc(-c3cnc(C4CC4)n3C)c2c1N] 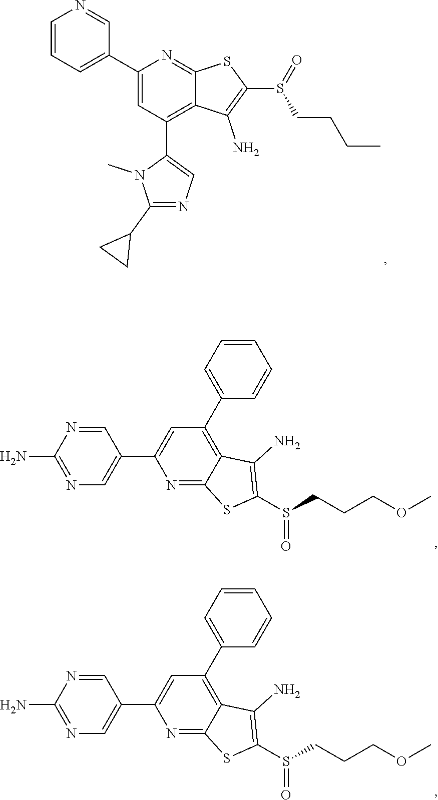 [SMILES: CCCC[S@@](=O)c1sc2nc(-c3cccnc3)cc(-c3cnc(C4CC4)n3C)c2c1N ; COCCC[S@@](=O)c1sc2nc(-c3cnc(N)nc3)cc(-c3ccccc3)c2c1N ; COCCC[S@](=O)c1sc2nc(-c3cnc(N)nc3)cc(-c3ccccc3)c2c1N] 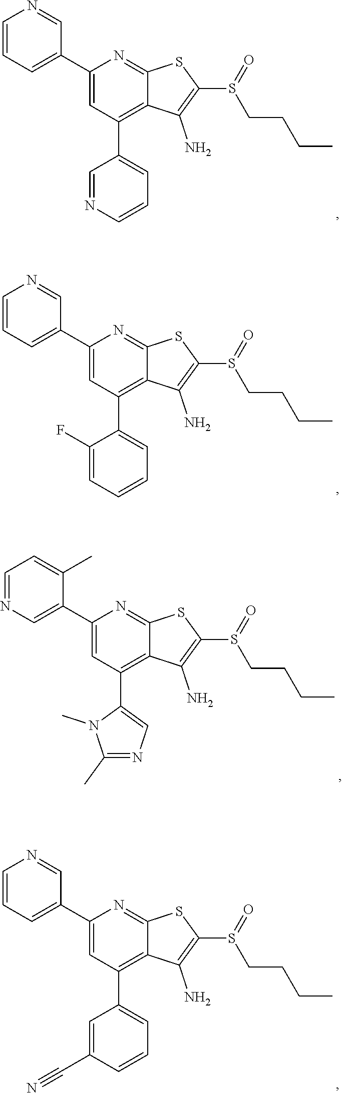 [SMILES: CCCCS(=O)c1sc2nc(-c3cccnc3)cc(-c3cccnc3)c2c1N ; CCCCS(=O)c1sc2nc(-c3cccnc3)cc(-c3ccccc3F)c2c1N ; CCCCS(=O)c1sc2nc(-c3cnccc3C)cc(-c3cnc(C)n3C)c2c1N ; CCCCS(=O)c1sc2nc(-c3cccnc3)cc(-c3cccc(C#N)c3)c2c1N] 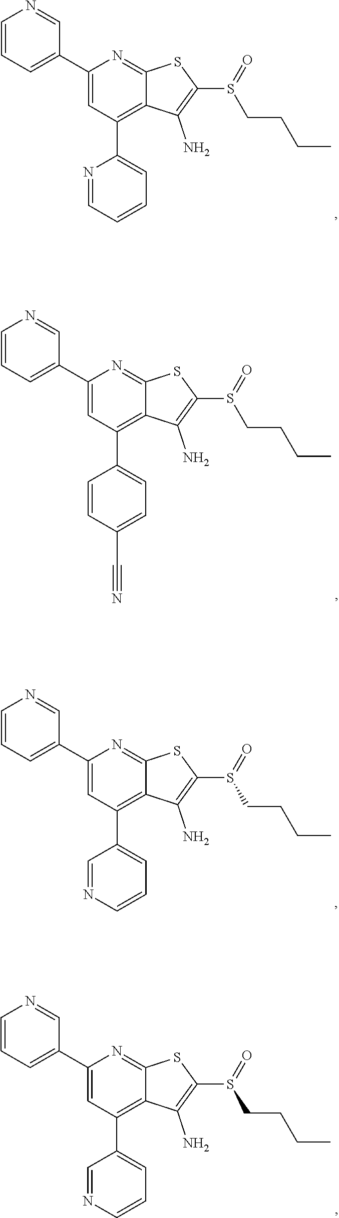 [SMILES: CCCCS(=O)c1sc2nc(-c3cccnc3)cc(-c3ccccn3)c2c1N ; CCCCS(=O)c1sc2nc(-c3cccnc3)cc(-c3ccc(C#N)cc3)c2c1N ; CCCC[S@@](=O)c1sc2nc(-c3cccnc3)cc(-c3cccnc3)c2c1N ; CCCC[S@](=O)c1sc2nc(-c3cccnc3)cc(-c3cccnc3)c2c1N] 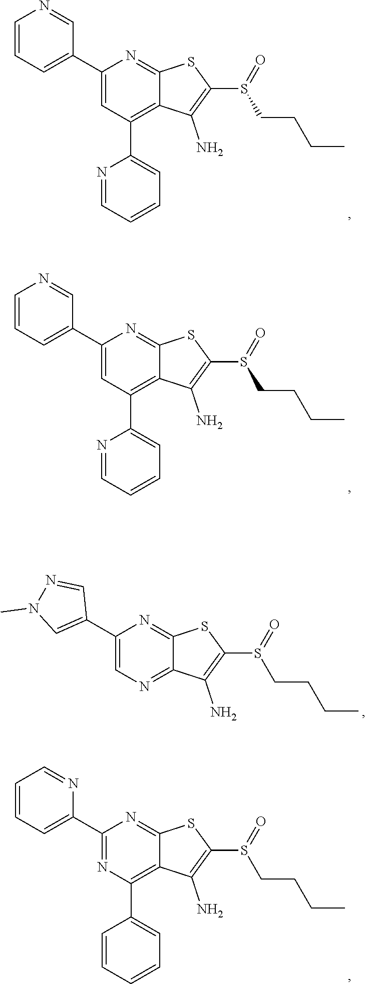 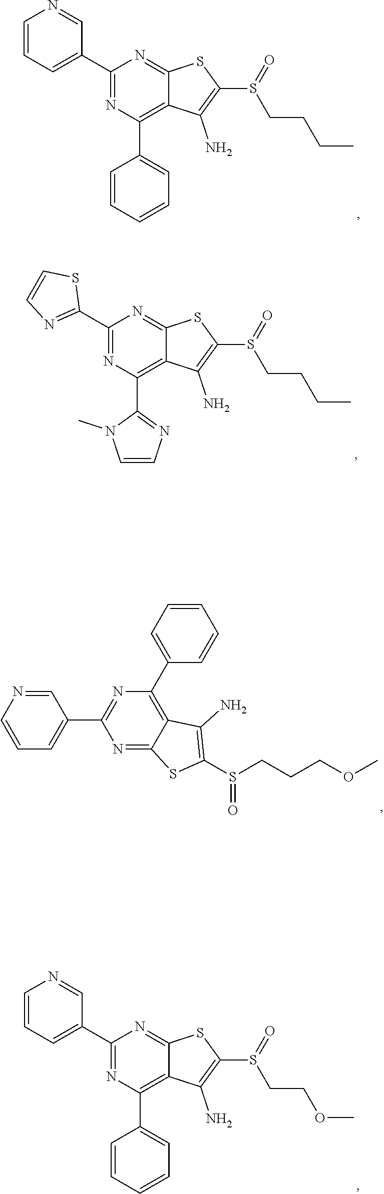 [SMILES: CCCCS(=O)c1sc2nc(-c3cccnc3)nc(-c3ccccc3)c2c1N ; CCCCS(=O)c1sc2nc(-c3nccs3)nc(-c3nccn3C)c2c1N ; COCCCS(=O)c1sc2nc(-c3cccnc3)nc(-c3ccccc3)c2c1N ; COCCS(=O)c1sc2nc(-c3cccnc3)nc(-c3ccccc3)c2c1N] 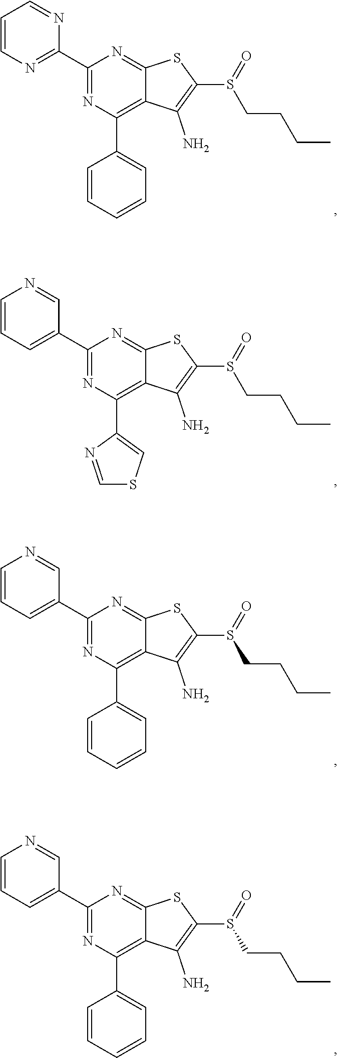 [SMILES: CCCCS(=O)c1sc2nc(-c3ncccn3)nc(-c3ccccc3)c2c1N ; CCCCS(=O)c1sc2nc(-c3cccnc3)nc(-c3cscn3)c2c1N ; CCCC[S@](=O)c1sc2nc(-c3cccnc3)nc(-c3ccccc3)c2c1N ; CCCC[S@@](=O)c1sc2nc(-c3cccnc3)nc(-c3ccccc3)c2c1N] 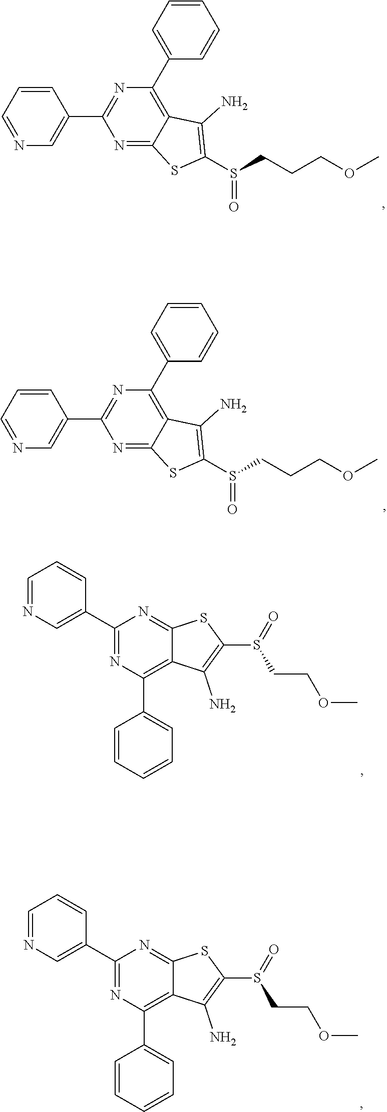 [SMILES: COCCC[S@@](=O)c1sc2nc(-c3cccnc3)nc(-c3ccccc3)c2c1N ; COCCC[S@](=O)c1sc2nc(-c3cccnc3)nc(-c3ccccc3)c2c1N ; COCC[S@@](=O)c1sc2nc(-c3cccnc3)nc(-c3ccccc3)c2c1N ; COCC[S@](=O)c1sc2nc(-c3cccnc3)nc(-c3ccccc3)c2c1N] 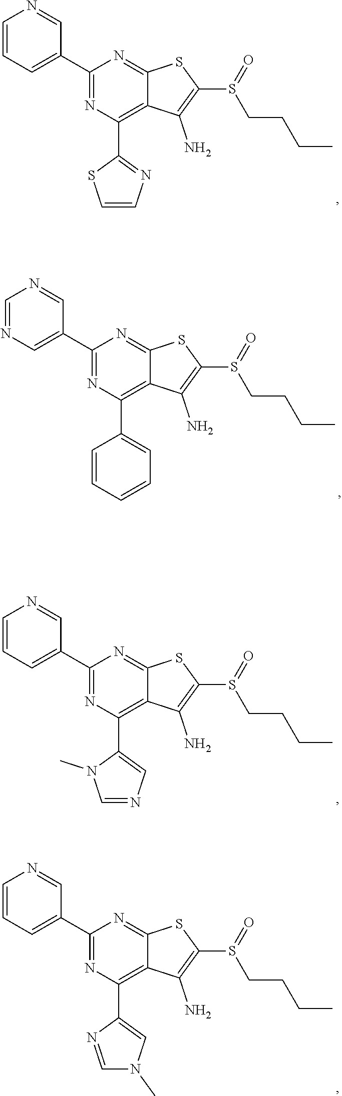 [SMILES: CCCCS(=O)c1sc2nc(-c3cccnc3)nc(-c3nccs3)c2c1N ; CCCCS(=O)c1sc2nc(-c3cncnc3)nc(-c3ccccc3)c2c1N ; CCCCS(=O)c1sc2nc(-c3cccnc3)nc(-c3cncn3C)c2c1N ; CCCCS(=O)c1sc2nc(-c3cccnc3)nc(-c3cn(C)cn3)c2c1N] 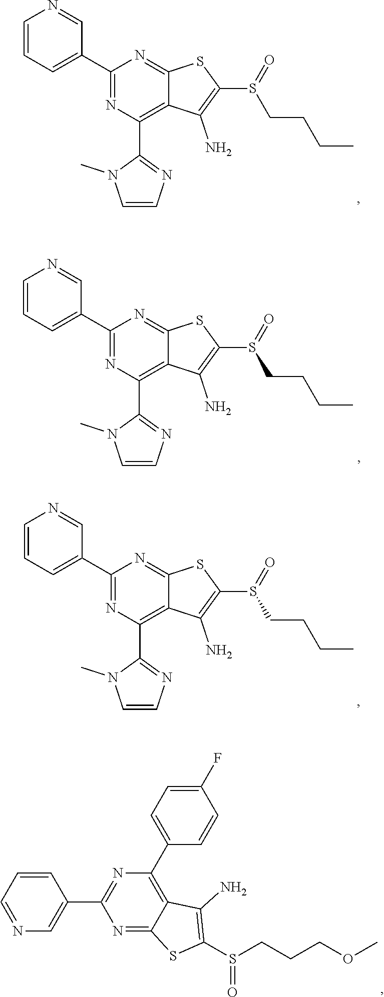 [SMILES: CCCCS(=O)c1sc2nc(-c3cccnc3)nc(-c3nccn3C)c2c1N ; CCCC[S@](=O)c1sc2nc(-c3cccnc3)nc(-c3nccn3C)c2c1N ; CCCC[S@@](=O)c1sc2nc(-c3cccnc3)nc(-c3nccn3C)c2c1N ; COCCCS(=O)c1sc2nc(-c3cccnc3)nc(-c3ccc(F)cc3)c2c1N] 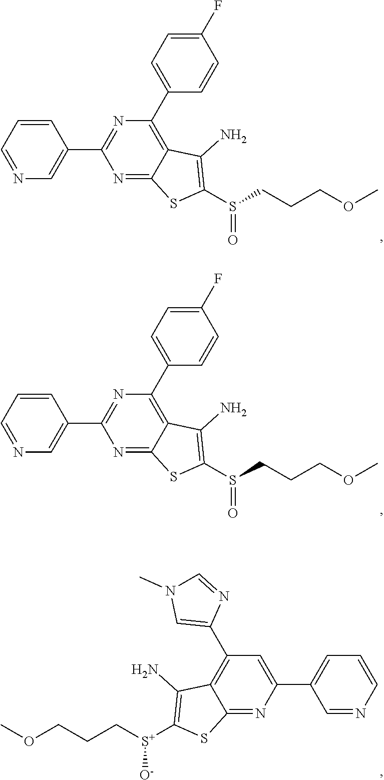 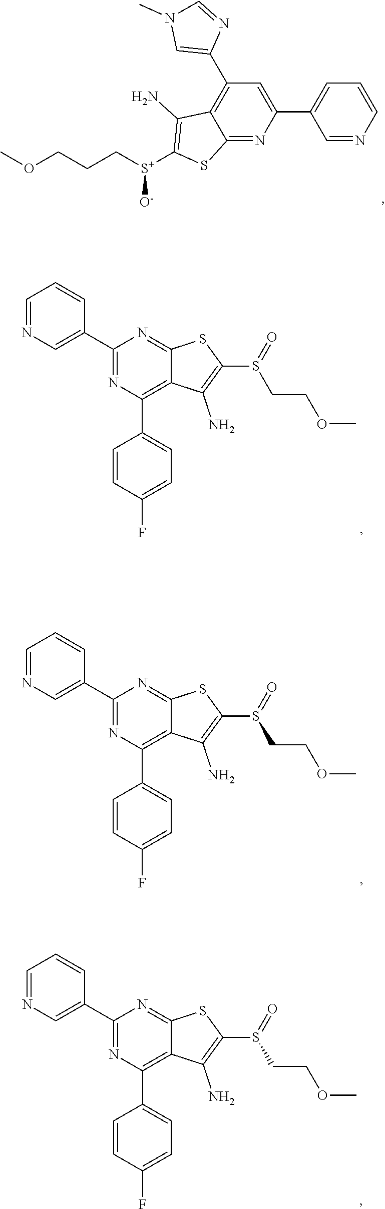 [SMILES: COCCC[S@+]([O-])c1sc2nc(-c3cccnc3)cc(-c3cn(C)cn3)c2c1N ; COCCS(=O)c1sc2nc(-c3cccnc3)nc(-c3ccc(F)cc3)c2c1N ; COCC[S@](=O)c1sc2nc(-c3cccnc3)nc(-c3ccc(F)cc3)c2c1N ; COCC[S@@](=O)c1sc2nc(-c3cccnc3)nc(-c3ccc(F)cc3)c2c1N] 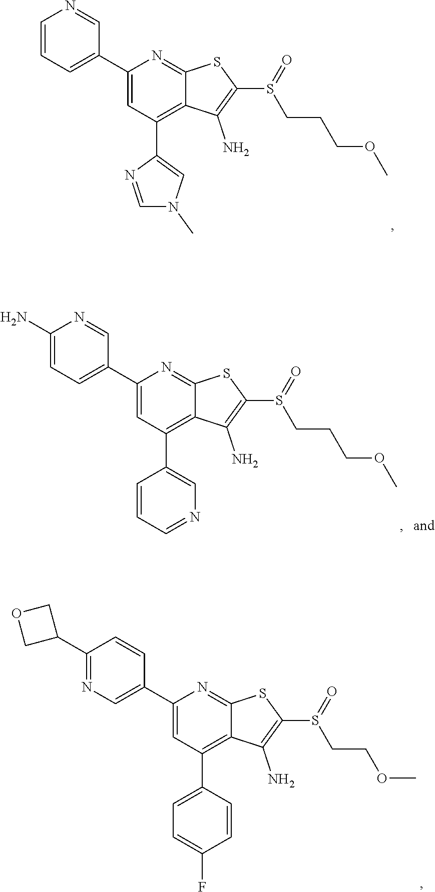 [SMILES: COCCCS(=O)c1sc2nc(-c3cccnc3)cc(-c3cn(C)cn3)c2c1N ; COCCCS(=O)c1sc2nc(-c3ccc(N)nc3)cc(-c3cccnc3)c2c1N ; COCCS(=O)c1sc2nc(-c3ccc(C4COC4)nc3)cc(-c3ccc(F)cc3)c2c1N]
or a pharmaceutically acceptable salt thereof.

3. A method of treating or preventing a fibrotic disease, disorder, or condition comprising administering to a subject in need thereof a therapeutically effect amount of a compound as provided in claim 1.
